(19) United States
(12) Patent Application Publication
(10) Pub. No.: US 2025/0326732 A1
(43) Pub. Date: Oct. 23, 2025

(54) CHROMEN-2-ONE MODULATORS OF POLRMT

(71) Applicant: Pretzel Therapeutics, Inc., Waltham, MA (US)

(72) Inventors: Gabriel Martinez Botella, Wayland, MA (US); Jeremy Green, Waltham, MA (US); Paul S. Charifson, Carrabassett Valley, ME (US); Simon Giroux, Cambridge, MA (US); Andrew Griffin, L'Île Bizard, Quebec (CA)

(73) Assignee: Pretzel Therapeutics, Inc., Waltham, MA (US)

(21) Appl. No.: 18/687,562

(22) PCT Filed: Aug. 30, 2022
(86) PCT No.: PCT/US2022/042099

§ 371 Date: Feb. 28, 2024

Related U.S. Application Data
(60) Provisional application No. 63/238,753, filed on Aug. 30, 2021.
(60) Provisional application No. 63/276,267, filed on Nov. 5, 2021.
(60) Provisional application No. 63/284,890, filed on Dec. 1, 2021.
(60) Provisional application No. 63/358,734, filed on Jul. 6, 2022.

(51) Int. Cl. C07D 311/18; A61K 31/37; A61K 31/397; A61K 31/4025; A61K 31/41; A61K 31/4155; A61K 31/4178; A61K 31/422; A61K 31/427; A61K 31/4433; A61K 31/453; A61K 31/5377; A61K 31/541; C07B 59/00; C07D 405/06; C07D 405/12; C07D 407/12; C07D 413/12; C07D 417/12

(52) U.S. Cl. CPC C07D 311/18; A61K 31/37; A61K 31/397; A61K 31/4025; A61K 31/41; A61K 31/4155; A61K 31/4178; A61K 31/422; A61K 31/427; A61K 31/4433; A61K 31/453; A61K 31/5377; A61K 31/541; C07B 59/002; C07D 405/06; C07D 405/12; C07D 407/12; C07D 413/12; C07D 417/12

(57) ABSTRACT

The present invention provides novel chromen-2-one compounds that are inhibitors of mitochondrial RNA polymerase for treating various diseases such as cancer and others associated with metabolic disorders and mitochondrial dysfunction.

### CROSS-REFERENCE TO RELATED APPLICATIONS

This application is the National Stage entry under 35 U.S.C. § 371 of International Application No. PCT/US2022/042099, filed on Aug. 30, 2022, published on Mar. 9, 2023 under Publication Number WO 2023/034346 A1, which claims priority from U.S. Provisional Application No. 63/238,753, filed Aug. 30, 2021, U.S. Provisional Application No. 63/276,267, filed Nov. 5, 2021, U.S. Provisional Application No. 63/284,890, filed Dec. 1, 2021, and U.S. Provisional Application No. 63/358,734, filed Jul. 6, 2022, all of the applications identified above are incorporated herein by reference in their entirety.

### FIELD OF THE INVENTION

The present invention relates to novel POLRMT modulators, their prodrugs, their pharmaceutically acceptable salts, and pharmaceutical compositions thereof. The present invention also relates to methods of using such compounds and compositions, including to inhibit or promote POLRMT, and to treat various neurodegenerative and metabolic disorders, cancer, and also disorders related to aging and mitochondrial diseases.

### BACKGROUND OF THE INVENTION

Human mitochondrial RNA polymerase, POLRMT (also referred to as h-mtRNAP), is a nuclear-encoded single-subunit DNA-dependent RNA polymerase. POLRMT is 1230 amino acids in length and consists of three distinct regions: (1) a C-terminal polymerase domain (CTD) (residues 648-1230); (2) an N-terminal domain (NTD) (residues 369-647); and (3) an N-terminal extension (NTE) (residues 1-368). See, e.g., Arnold, J. J., et al., “Human mitochondrial RNA polymerase: Structure-function, mechanism and inhibition,” Biochim. Biophys. Acta, 1819, 948-960 (2012). It is structurally related to the single-subunit RNA polymerase encoded by bacteriophage T7. The CTD is also known as the catalytic domain due to its function of catalyzing nucleotide incorporation into a growing RNA molecule during transcription. This domain is highly conserved across species, whereas by contrast the NTE demonstrates significant sequence variability, suggesting organism-specific roles for this domain of POLRMT. Regarding the POLRMT NTD, structurally it resembles the N-terminal domain (also called the promoter-binding domain) of T7 RNA polymerase. However, for promoter-specific transcription initiation POLRMT requires assistance from additional transcription factors, whereas T7 RNA polymerase does not.

A primary biological role of POLRMT is to transcribe the mitochondrial genome to produce the RNAs needed for expression of mitochondrial DNA (mtDNA). Initiation, elongation, and termination are the three steps of mitochondrial transcription. Each of a light-strand promoter (LSP) and two heavy-strand promoters (HSP-1 and HSP-2) on the mtDNA contains a transcription initiation site. See, e.g., Basu, U., et al., “Structure, mechanism, and regulation of mitochondrial DNA transcription initiation,” J. Biol. Chem., 295 (52), 18406-425 (2020). For promoter-specific transcription initiation, POLRMT requires two transcription factors, TFAM (transcription factor A mitochondrial) and TFB2M (transcription factor B mitochondrial). See id. Various models suggest different mechanisms by which the initiation complex structure with POLRMT, TFAM, and TFB2M comes together to cover the promoter DNA for initiation of transcription. In one current model TFAM recruits POLRMT to the promoter site to form a protein-protein pre-initiation complex, to which TFB2M binds to form the initiation complex, which covers the promoter DNA. See id. During initiation, the RNA is elongated to about 8-10 nucleotides in length. Conformational changes occur at that point, including promoter release and displacement of the initiation factors, converting the initiation complex into an elongation complex at which time transcription occurs. See id.

The mitochondrial genome encodes the various subunits of the electron transport chain. See, e.g., Shokolenko, I. N., et al., “Maintenance and expression of mammalian mitochondrial DNA,” Annu. Rev. Biochem., 85, 133-160 (2016). Specifically, transcription of the mitochondrial genome is necessary for the expression of 13 subunits of the oxidative phosphorylation (OXPHOS) system, as well as two rRNAs and 22 tRNAs. See, e.g., Shokolenko, I. N., et al., “Mitochondrial transcription in mammalian cells,” Frontiers in Bioscience, Landmark, 22, 835-853 (2017). Thus, POLRMT is essential for biogenesis of the OXPHOS system, resulting in ATP production. This, in turn, is vital for energy homeostasis in the cell.

Dysregulation of POLRMT and the OXPHOS system have been implicated in various disease states, in particular cancer. Cancer is now the second leading cause of death in the United States, with projections indicating that almost two million new cases will be diagnosed in 2022 and over 600,000 deaths will be the result of cancer. See Siegel, R. L. et al., “Cancer statistics 2022.” CA Cancer J. Clin. (72) 7-33 (2022). High rates of OXPHOS have been shown to support growth in cancer cell lines, including in a subset of diffuse large B cell lymphoma cells. See, e.g., DeBeradinis, R. J., “A mitochondrial power play in lymphoma,” Cancer Cell, 22, 423-24 (2012). Noteworthy is the observation that metabolic heterogeneity exists not only between different types of cancer, but also among tumors of the same type. Similarly, in a study using melanoma cell lines representative of various stages of tumor progression and that collectively mimic the mixture of cells found in a tumor, it was found that metastatic cells demonstrated a high OXPHOS capacity. Rodrigues, M. F., et al., “Enhanced OXPHOS, glutaminolysis and β-oxidation constitute the metastatic phenotype of melanoma cells,” Biochem. J. 473:703-715 (2016). These data suggest mitochondria play a role as cells progress toward metastasis, possibly to provide the energy needed for tumor cell migration and invasion.

Relatedly, overexpression of POLRMT has been linked to multiple types of cancers, suggesting that it plays a role in tumor growth. Supporting this hypothesis is, for example, a study involving acute myeloid leukemia (AML) cells, which are known to have high oxidative phosphorylation and mitochondrial mass, as well as low respiratory chain spare reserve capacity. POLRMT knockdown AML cells demonstrated a reduction in POLRMT levels, decreased oxidative phosphorylation, and increased cell death as compared to control AML cells. See Bralha, F. N., et al., “Targeting mitochondrial RNA polymerase in acute myeloid leukemia,” Oncotarget, 6 (35), 37216-228 (2015). In other work, injection into nude mice of a human breast cancer cell line that overexpresses POLRMT resulted in increased tumor growth, independent of tumor angiogenesis, suggesting that POLRMT should be considered a tumor promoter or metabolic oncogene. Salem, A. F., et al. “Mitochondrial biogenesis in epithelial cancer cells promotes breast cancer tumor growth and confers autophagy resistance,” Cell Cycle, 11 (22), 4174-80 (2012). Recently, the expression of POLRMT in non-small cell lung cancer (NSCLC) has been examined. See Zhou, T. et al., “The requirement of mitochondrial RNA polymerase for non-small cell lung cancer cell growth,” Cell Death and Disease, 12, 751 (2021). While POLRMT mRNA and protein were detected in normal human lung tissue, their levels were significantly higher in cancer tissue. Similar results were obtained when comparing primary lung epithelial cells to NSCLC cells. Using short hairpin RNA (shRNA) to silence POLRMT mRNA and downregulate POLRMT protein resulted in inhibition of NSCLC cell viability, proliferation, migration, and invasion. Moreover, silencing of POLRMT significantly induced apoptosis activation in both primary and established NSCLC cells. Injection of POLRMT shRNA in an adeno-associated virus construct into tumors effectively inhibited NSCLC xenograft growth in mice. Taken together, these data suggest that POLRMT could be an oncogenic gene for NSCLC. 0008 The development of multidrug resistance (MDR) to numerous cancers is associated with poor prognosis and presents significant challenges in the treatment of this disease. Because such resistance encompasses drugs having different structures and mechanisms of action, identifying and targeting a single biochemical pathway that could re-sensitize MDR cancer cells to established chemotherapy would provide a promising treatment strategy. See Yu, H.-J., “Targeting mitochondrial metabolism and RNA polymerase POLRMT to overcome multidrug resistance in cancer,” Front. Chem., 9:775226 (2021). A main reason for the development of MDR is enhanced drug efflux from and decreased drug accumulation in MDR cells due to ATP-dependent protein transporters that pump drugs out of cells. Inhibiting POLRMT and consequently the production of the proteins essential for the OXPHOS system could compromise ATP production and, in turn, the ATP-dependent efflux of chemotherapeutic agents from cancer cells.

Consistent with the findings that the OXPHOS system and POLRMT may be involved in the etiology of and in some cases overexpressed in some cancers, small-molecule inhibitors of POLRMT have been developed. See, e.g., EP 3 598 972 A1; WO 2019/057821 A1; and WO 2020/188049 A1. Some of these inhibitors have been shown to be useful in inhibiting cancer cell proliferation without affecting control cells. See Bonekamp, N. A., et al., “Small-molecule inhibitors of human mitochondrial DNA transcription,” Nature, 588, 712-716 (2020). The cancer cell toxicity was correlated to a considerable increase in the levels of mono- and diphosphate nucleotides with a concomitant decrease in nucleotide triphosphate levels, all the result of a debilitated OXPHOS system. Similarly, treatment with POLRMT inhibitors caused a decrease in citric-acid cycle intermediates and ultimately cellular amino acid levels, the result of which is a state of severe energy and nutrient depletion. See id. Such inhibitors also produced a decrease in tumor volume in mice with no significant toxicity in control animals. Specifically, mtDNA transcript levels in tumor cells were decreased as compared to transcript levels in differentiated tissue. These data highlight the importance of mtDNA expression in rapidly dividing cells as opposed to post-mitotic tissue, a distinction that may be capitalized on using POLRMT inhibitors that are capable of modulating mtDNA transcription and ultimately the OXPHOS system.

While mitochondria are an emerging target for cancer treatment, the resistance mechanisms induced by chronic inhibition of mitochondrial function are poorly understood. In view of the challenges presented by drug resistance in cancer chemotherapy, the development of such resistance to small molecule inhibitors of POLRMT has been investigated. See Mennuni, M. et al., “Metabolic resistance to the inhibition of mitochondrial transcription revealed by CRISPER-Cas9 screen,” EMBO reports, 23: e53054 1-18 (2022). Using a CRISPR-Cas9 whole-genome screen, loss of genes belonging to von Hippel-Lindau (VHL) and mammalian target of rapamycin complex 1 (mTORC1) were the pathways that caused resistance to acute treatment with a POLRMT inhibitor. See id. at pp. 1-2. Moreover, dose-escalated chronic treatment of cells with this molecule resulted in drug-resistant cells that had increased levels of mtDNA, thereby giving rise to increased levels of mitochondrial transcripts and proteins. See id. at p. 5. The drug-resistant cells maintained higher levels of nucleotide levels, tricarboxylic acid cycle intermediates, and amino acids. See id. at p. 7. Notably, the drug-resistant cells did not have mutations in POLRMT that compromise inhibitor binding to the polymerase. See id. The development of resistance to POLRMT inhibitors underscores the importance and need for the development of other POLRMT inhibitors to understand and treat cancers of varying types.

Alterations in the OXPHOS system also have been implicated in the development of insulin resistance and ultimately Type-2 diabetes. In studies involving apoptosis inducing factor (AIF) knockout mice, a primary OXPHOS defect that produced OXPHOS deficiency revealed an increase in insulin sensitivity and resistance to diabetes and obesity. See Pospisilik, J. A., et al., “Targeted deletion of AIF decreases mitochondrial oxidative phosphorylation and protects from obesity and diabetes,” Cell, 131, 476-91 (2007). Correlated with these phenotypic changes were the metabolic alterations of increased glucose uptake and enhanced fuel utilization. Manipulation of the OXPHOS system with POLRMT modulators affords the potential for further understanding the physiological mechanisms involved in diseases such as diabetes and for the development of novel treatments for intervention of such metabolic disorders.

In addition to its critical role in transcription, POLRMT acts as the primase for mtDNA replication, thus playing a part in the regulation of mtDNA levels. Human mtDNA is a circular double-stranded DNA that is packaged in DNA-protein structures called mitochondrial nucleoids, for which TFAM is the most abundant structural component. See, e.g., Filograna, R., et al., “Mitochondrial DNA copy number in human disease: the more the better?” FEBS Letters, 595, 976-1002 (2021). TFAM facilitates mtDNA compaction, which results in regulating the accessibility of the DNA to cellular replication and transcription components. With respect to mtDNA replication, POLRMT is part of the mtDNA replisome along with the hexameric helicase TWINKLE, the heterotrimeric DNA polymerase gamma (POLγ) and the tetrameric mitochondrial single-stranded DNA-binding protein (mtSSB). See id. Its function in this replisome is to synthesize the RNA primers required for the initiation of the synthesis of both strands of mtDNA. While there may be many mechanisms by which mtDNA levels may be regulated, including modulation of POLRMT, what is known to date is that mtDNA copy number can be manipulated through modulation of TFAM expression.

While the correlation is not completely straightforward, changed levels of mtDNA have been implicated in neurogenerative disorders, cancer, and aging. See e.g., Filograna, R., et al., “Mitochondrial DNA copy number in human disease: the more the better?” FEBS Letters, 595, 976-1002 (2021). Particularly challenging is the attempt to understand the relationship between mtDNA copy number and cancer. It appears that such copy number can correlate with both increased and decreased disease burden. As such, tumor type and stage of disease may be important factors in determining the role of mtDNA copy number in the diagnosis and/or prognosis of cancer. With respect to aging, most data show a reduction in mtDNA levels in the older population. That being said, other study data are inconsistent as to the relationship between mtDNA copy number and longevity. By contrast, there appears to be a clearer correlation between neurodegeneration in Alzheimer's disease and reduction in mtDNA levels. Complicating the understanding of the relationship between mtDNA levels and disease is the role that mtDNA mutations have on various disorders. While accumulation of mtDNA mutations appears to occur in almost all types of cancer, it is unclear whether such mutations are causative of the cancer or merely a by-product of rapid replication in fast-dividing cells. Nonetheless, since POLRMT plays a key role in mtDNA replication, POLMRT modulation may provide an effective mechanism by which to understand various disease states and how to slow or alter the progression of disease.

Mutations affecting POLRMT may also cause human disease. See Oláhová, M., et al., “POLRMT mutations impair mitochondrial transcription causing neurological disease.” Nat. Commun., 12, 1135 (2021). POLRMT variants have been identified in a number of unrelated families. Patients present with multiple phenotypes, including global developmental delay, hypotonia, short stature, and speech/intellectual disability in childhood. POLRMT modulation may provide a mechanism to slow or alter the progression of disease.

POLRMT is of fundamental importance for both expression and replication of the human mitochondrial genome. While aspects of POLRMT biochemistry are known, its full physiological role in mitochondrial gene expression and homeostasis, as well as its underlying impact in the etiology of various disease states, remains unclear. Its dysfunction and/or deregulation impacts mitochondrial metabolism, sometimes through the OXPHOS system, which ultimately contributes to many metabolic, degenerative and age-related diseases such as cancer, diabetes, obesity, and Alzheimer's disease. Pharmacological inhibition of POLRMT is one means by which to gain a further understanding of the role of this polymerase in cell physiology and the development of disease. Regulation of metabolic mechanisms, including oxidative phosphorylation, with POLRMT modulators affords an opportunity for intervention in complex disorders. In view of the numerous and varied roles of POLRMT, the need exists for potent and specific modulators of POLRMT.

### SUMMARY OF THE INVENTION

Provided are compounds, pharmaceutically acceptable salts of the compounds, and prodrugs of the compounds; pharmaceutical compositions comprising the compounds or their salts or prodrugs; and methods of using the compounds, salts of the compounds, prodrugs of the compounds, or pharmaceutical compositions of the compounds, their salts, or their prodrugs to treat various neurodegenerative and metabolic disorders, cancer, and also disorders related to aging and mitochondrial diseases. The compounds and their pharmaceutically acceptable salts are particularly useful as modulators of POLRMT.

In one embodiment, the present invention is directed to a compound, a prodrug thereof, or a pharmaceutically acceptable salt thereof, represented by formula (I):

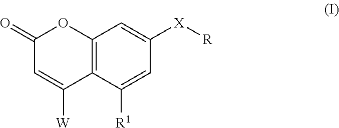

wherein:
X is C(R³)(R³), C₃-C₆ cycloalkyl, C(R³)(R³)O, C(R³)(R³)OC(O), C(R³)═C(R³), N(R⁴), C(R³)(R³)N(R³), C(O)(NR³), or C(O);
W is C₆-C₁₂ aryl or 5-12 membered heteroaryl, either of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium;
R is hydrogen, NR³R⁴, C(O)NR³R⁴, C₁-C₄ alkylcarboxyl, C(O)R³, carboxyl, CH₂CH₂R², hydroxyl, or C₁-C₆ alkoxyl optionally substituted with one or more groups, each independently selected from the group consisting of hydroxyl, fluoro, C₁-C₄ alkoxyl, NR³R⁴, C₁-C₄ alkylcarboxyl, carboxyl, and C(O)NR³R⁴,
or R is NR³C(O)R³, C₃-C₆ cycloalkyl optionally substituted with C(O)NR³R³, or C₄-C₆ heterocyclic,
or R is C₁-C₆ alkyl optionally substituted with one or more groups, each independently selected from the group consisting of hydroxyl, fluoro, cyano, C₁-C₄ alkoxyl, C₁-C₄ haloalkoxyl, phenyl, NR³R⁴, C₁-C₄ alkylcarboxyl, carboxyl, CR⁵R⁶—CO₂R⁷, C(O)NR³R⁴, C(O)NR³SO₂R³, NR³C(O)R³, C₃-C₆ cycloalkyl, 5- to 6-membered heterocyclic ring optionally substituted with R³, and heteroaryl optionally substituted with C₁-C₄ alkyl, hydroxyl, or oxo,
or R is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, and C₁-C₄ alkoxyl;
R¹ is hydrogen, hydroxyl, cyano, C₁-C₃ alkyl optionally substituted with one or more fluoro, C₃-C₄ cycloalkyl, or methoxyl optionally substituted with one to three fluoro;
R² is C₆-C₁₀ aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R² is C₁-C₄ haloalkoxyl, OR³, NR³R⁴, C(O)R³, or C(O)NR³R⁴;
each R³ is independently hydrogen, or C₃-C₆ cycloalkyl, C₄-C₆ heterocyclic, C₁-C₄ alkyl, each optionally substituted with one or more fluoro or carboxyl;
each R⁴ is independently R³, C(O) C₁-C₄ alkyl optionally substituted with one or more fluoro groups, C(O)NHC₁-C₄ alkyl optionally substituted with one or more fluoro groups, or C₁-C₄ alkyl optionally substituted with one or more deuterium, hydroxyl, fluoro, C₁-C₃ alkoxyl, NR³R³, carboxyl, or C(O)NR³R³;

or if R³ and R⁴ are attached to the same nitrogen atom, R³ and R⁴ together with their connecting nitrogen form a 4- to 6-membered heterocyclic ring optionally containing another heteroatom that is N, O, or S, and such heterocyclic ring is optionally substituted with one or more groups each independently selected from the group consisting of fluoro, chloro, cyano, carboxyl, C(O)NR³R³, NR³R⁴, NR¹C(O)R³, S(O)₂R³, S(O)₂NR³R³, C₁-C₄ alkylcarboxylate, and C₁-C₄ alkyl optionally substituted with hydroxyl, C₁-C₄ alkoxy, or NR¹C(O)R³;
R⁵ and R⁶ together with their connecting carbon form a 3- to 5-membered cyclic or heterocyclic ring; and
R⁷ is H or NH₂.

Further embodiments of the present invention are compounds of the invention (that is, compounds of formula (I)), prodrugs of the compounds, or their pharmaceutically acceptable salts wherein one or more hydrogen is substituted with a deuterium atom.

Additional embodiments of the invention are pharmaceutical compositions comprising a compound of the invention or a pharmaceutically acceptable salt thereof, or a prodrug thereof and one or more pharmaceutically acceptable excipients.

Further embodiments of the invention are methods of treating a disease, such methods comprising administering to a subject in need thereof a therapeutically effective amount of a compound of the invention, a prodrug thereof, or a pharmaceutically acceptable salt thereof. In some embodiments, the disease is selected from the group consisting of adrenal gland cancer, anal cancer, adenocarcinoma, angiosarcoma, bile duct cancer, bladder cancer, blastic plasmacytoid dendritic cell neoplasm, bone cancer, brain cancer, breast cancer, bronchogenic carcinoma, central nervous system (CNS) cancer, cervical cancer, cholangiocarcinoma, chondrosarcoma, colon cancer, colorectal cancer, cancer of connective tissue, esophageal cancer, embryonal carcinoma, fibrosarcoma, gall bladder cancer, gastric cancer, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas), melanoma, Merkel cell carcinoma, mesothelioma, multiple myeloma, muscular cancer, myxosarcoma, neuroblastomas, non-small cell lung cancer, ocular cancer, oral/digestive tract cancer, osteogenic sarcoma, ovarian cancer, papillary carcinoma, pancreatic cancer, rhabdomyosarcoma, polycythemia vera, prostate cancer, renal cancer, retinal cancer, skin cancer, small cell lung carcinoma, stomach cancer, testicular cancer, throat cancer, thyroid cancer, uterine cancer, vaginal cancer, and vulvar cancer. In some embodiments, the disease is selected from the group consisting of Alzheimer's disease and Parkinson's disease. In some embodiments, the disease is selected from the group consisting of obesity, diabetes, non-alcoholic steatohepatitis (NASH), and related metabolic syndromes such as non-alcoholic fatty liver disease (NAFLD). In some embodiments, the disease is related to aging or a mitochondrial disorder.

Additional embodiments of the invention are methods of treating neurodegenerative disorders and metabolic disorders, such as those identified in Bonekamp, N. A. et al. “Small-molecule inhibitors of human mitochondrial DNA transcription,” Nature, 588, 712-716 (2020), Filograna, R. et al, “Mitochondrial DNA copy number in human disease: the more the better?” FEBS Lett., 595, 976-1002 (2021), Wrendenber, A. et al. “Respiratory chain dysfunction in skeletal muscle does not cause insulin resistance,” Biochem. Biophys. Res. Comm., 350, 202-207 (2006), Pospililik, J. A. et al. “Targeted deletion of AIF decreases mitochondrial oxidative phosphorylation and protects from obesity and diabetes,” Cell, 131, 476-491 (2007), and PCT International Publication No. WO 2019/057821 A1 and references therein.

Further embodiments of the invention are methods of treating disease of aging.

### DETAILED DESCRIPTION OF THE INVENTION

Modulators of POLRMT are useful in compositions and methods suitable for treating many disorders, such as cancer, neurodegenerative disorders, metabolic disorders, as well as diseases related to aging and mitochondrial diseases. Provided herein are compounds of formula (I), pharmaceutically acceptable salts thereof, prodrugs thereof, and pharmaceutical compositions comprising such compounds, their salts, or their prodrugs that are useful in treating a condition or disease, such as cancer, neurodegenerative disorders, and metabolic disorders.

### Definitions

The term “alkyl” as used herein refers to both branched- and straight-chain saturated aliphatic hydrocarbon groups having the specified number of carbon atoms in a specified range. For example the term “C₁-C₆ alkyl” means linear or branched chain alkyl groups, including all possible isomers, having 1, 2, 3, 4, 5, or 6 carbon atoms.

The term “alkoxy” or “alkoxyl” as used herein refers to an —O-alkyl group. For example, the term “C₁-C₄ alkoxyl” means —O—C₁-C₄ alkyl. Examples of alkoxyl include methoxyl, ethoxyl, propoxyl (e.g., n-propoxyl and isopropoxyl), and the like.

The term “haloalkoxy” or “haloalkoxyl” as used herein refers to an —O-alkyl group in which at least one of the hydrogen atoms of the alkyl group is replaced with a halogen atom. Examples of haloalkoxyl include trifluoromethoxyl, 2,2,2-trifluoroethoxyl, and the like.

The term “alkanoyl” or “acyl” as used herein refers to an C(O)-alkyl group. For example, the term “C₁-C₆ alkanoyl” means C(O)—C₁-C₆ alkyl. Examples of alkanoyl include acetyl, propionyl, butyryl, and the like.

The term “bicyclic” as used herein refers to a saturated or unsaturated 6- to 12-membered ring consisting of two joined cyclic substructures, and includes fused, bridged, and spiro bicyclic rings.

The term “heterobicyclic” as used herein refers to a bicyclic ring that contains 1 or more heteroatom(s) in one or more rings that are optionally substituted or oxidized, and may be selected from nitrogen (including N-oxides), oxygen, sulfur (including oxidized forms such as sulfones and sulfonates), phosphorus (including oxidized forms such as phosphates), boron, etc. Examples of heterobicyclic rings include, but are not limited to 8-azabicyclo[3.2.1]octan-8-yl, 3-oxa-8-azabicyclo[3.2.1]octan-8-yl, 8-oxa-3-azabicyclo[3.2.1]octan-3-yl, and 5-methyl-2,5-diazabicyclo[2.2.1]heptan-2-yl.

The term “cycloalkyl” as used herein refers to a cyclized alkyl ring having the indicated number of carbon atoms in a specified range. Thus, for example, “C₃-C₆ cycloalkyl” encompasses each of cyclopropyl, cyclobutyl, cyclopentyl, and cyclohexyl.

The term “aryl” as used herein refers to a monocyclic or fused bicyclic ring system having the characteristics of aromaticity, wherein at least one ring contains a completely conjugated pi-electron system. Typically, aryl groups contain 6 to 14 carbon atoms (“C₆-C₁₄ aryl”) or preferably, 6 to 12 carbon atoms (“C₆-C₁₂ aryl”). Fused aryl groups may include an aryl ring (e.g., a phenyl ring) fused to another aryl ring, or fused to a saturated or partially unsaturated carbocyclic or heterocyclic ring. The point of attachment to the base molecule on such fused aryl ring systems may be a C atom of the aromatic portion or a C or N atom of the non-aromatic portion of the ring system. Examples, without limitation, of aryl groups include phenyl, biphenyl, naphthyl, anthracenyl, indanyl, indenyl, and tetrahydronaphthyl.

The term “cycloaryl” herein refers to a polycyclic group wherein an aryl group is fused to a 5- or 6-membered aliphatic ring. For example, “C₆-C₁₂ cycloaryl” means a C₆-C₁₂ aryl fused to a 5- or 6-membered aliphatic ring.

The term “heteroaryl” as used herein refers to (i) a 5- or 6-membered ring having the characteristics of aromaticity containing at least one heteroatom selected from N, O and S, wherein each Nis optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatoms, at least one ring is aromatic, each Nis optionally in the form of an oxide, and each S in a ring which is not aromatic is optionally S(O) or S(O)₂. Typically, heteroaryl groups contain 5 to 14 ring atoms (“5-14 membered heteroaryl”), and preferably 5 to 12 ring atoms (“5-12 membered heteroaryl”). Heteroaryl rings are attached to the base molecule via a ring atom of the heteroaromatic ring, such that aromaticity is maintained. Suitable 5- and 6-membered heteroaromatic rings include, for example, pyridyl, 3-fluororopyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1,2-a]pyridinyl, benzotriazolyl, indazolyl, indolinyl, and isoindolinyl.

The term “heteroaryloxy” or “heteroaryloxyl” as used herein refers to an —O-heteroaryl group.

The term “heterocycle”, “heterocyclyl”, or “heterocyclic” as used herein represents a stable 3- to 10-membered monocyclic, non-aromatic ring that is either saturated or unsaturated, and that consists of carbon atoms and from one to four heteroatoms selected from the group consisting of N, O, and S. Examples include oxiranyl, aziridinyl, oxetanyl, azetidinyl, tetrahydrofuranyl, pyrrolidinyl, tetrahydropyranyl, piperidinyl, 1,4-dioxanyl, morpholinyl, piperazinyl, azepanyl, oxepanyl, tetrazole, and oxazepanyl.

The term “oxo” as used herein refers to a group which consists of oxygen which is double bonded to carbon or any other element.

The term “imine” as used herein refers to a group containing a carbon-nitrogen double bond.

The term “carboxyl” as used herein refers to a combination of two functional groups attached to a single carbon atom, namely, hydroxyl (OH) and carbonyl (O).

The term “optionally substituted” or “optional substituents” as used herein means that the groups are either unsubstituted or substituted with one or more of the substituents specified. When the groups are substituted with more than one substituent, the substituents may be the same or different. Furthermore, when using the terms “independently,” “independently are,” and “independently selected from” means that the groups may be the same or different.

The term “deuterium” as used herein refers to an isotope of hydrogen that has one proton and one neutron in its nucleus and that has twice the mass of ordinary hydrogen. Deuterium herein is represented by the symbol “D”.

The term “deuterated” by itself or used to modify a compound or group as used herein refers to the presence of at least one deuterium atom attached to carbon. For example, the term “deuterated compound” refers to a compound which contains one or more carbon-bound deuterium(s). In a deuterated compound of the present invention, when a particular position is designated as having deuterium, it is understood that the abundance of deuterium at that position is substantially greater than the natural abundance of deuterium, which is about 0.015%.

The term “undeuterated” or “non-deuterated” as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as “H” or “hydrogen”, the position is understood to have hydrogen at its natural abundance isotopic composition.

The term “isotopic enrichment factor” as used herein refers to the ratio between the isotope abundance and the natural abundance of a specified isotope.

The term “isotopologue” as used herein refers to a species in which the chemical structure differs from a specific compound of the invention only in the isotopic composition thereof.

The term “substantially free of other stereoisomers” as used herein means less than 10% of other stereoisomers, preferably less than 5% of other stereoisomers, more preferably less than 2% of other stereoisomers and most preferably less than 1% of other stereoisomers are present.

The term “pharmaceutically acceptable salt” as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be found in Stahl, P. H. et al. Pharmaceutical Salts: Properties, Selection, and Use, 2nd Revised Edition, Wiley, 2011.

The term “prodrug” as used herein refers to derivatives of compounds of the invention which may have reduced pharmacological activity, but can, when administered to a patient, be converted into the inventive compounds. Design and use of prodrugs may be found in “Pro-drugs as Novel Delivery Systems,” Vol. 14, ACS Symposium Series (T Higuchi and W Stella) and “Bioreversible Carriers in Drug Design,” Pergamon Press, 1987 (ed. E B Roche, American Pharmaceutical Association), the disclosures of which are incorporated herein by reference in their entireties. Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety. Some non-limiting examples of prodrugs in accordance with the invention include: (i) where the compound contains a carboxylic acid functionality-(COOH), an ester thereof, for example, replacement of the hydrogen with (C₁-C₆)alkyl; (ii) where the compound contains an alcohol functionality (—OH), an ether thereof, for example, replacement of the hydrogen with (C₁-C₆)alkanoyloxymethyl, or with a phosphate ether group; and (iii) where the compound contains a primary or secondary amino functionality (—NH₂ or —NHR, where R is not H), an amide thereof, for example, replacement of one or both hydrogens with C₁-C₆ alkanoyl. Further examples of replacement groups in accordance with the foregoing examples and examples of other prodrug types may be found in the aforementioned references.

The terms “treatment”, “treating” and “treat” as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term “therapeutically effective amount” as used herein refers to that amount of compound of the invention that will elicit the biological or medical response of a tissue, system, animal, or human that is being sought by a researcher, veterinarian, medical doctor or other. As will be recognized by a person of ordinary skill in the art, a therapeutically effective amount of the compounds of the invention will vary and will depend on the diseases treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

### Compounds

In one embodiment, the present invention is directed to a compound, a prodrug thereof, or a pharmaceutically acceptable salt thereof, represented by formula (I):

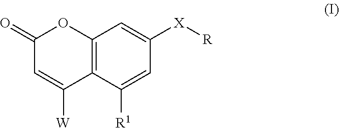

wherein:
wherein:
X is C(R³)(R³), C₃-C₆ cycloalkyl, C(R³)(R³)O, C(R³)(R³)OC(O), C(R³)═C(R³), N(R⁴), C(R³)(R³)N(R³), C(O)(NR³), or C(O);
W is C₆-C₁₂ aryl or 5-12 membered heteroaryl, either of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium;
R is hydrogen, NR³R⁴, C(O)NR³R⁴, C₁-C₄ alkylcarboxyl, C(O)R³, carboxyl, CH₂CH₂R², hydroxyl, or C₁-C₆ alkoxyl optionally substituted with one or more groups, each independently selected from the group consisting of hydroxyl, fluoro, C₁-C₄ alkoxyl, NR³R⁴, C₁-C₄ alkylcarboxyl, carboxyl, and C(O)NR³R⁴,
or R is NR³C(O)R³, C₃-C₆ cycloalkyl optionally substituted with C(O)NR³R³, or C₄-C₆ heterocyclic,
or R is C₁-C₆ alkyl optionally substituted with one or more groups, each independently selected from the group consisting of hydroxyl, fluoro, cyano, C₁-C₄ alkoxyl, C₁-C₄ haloalkoxyl, phenyl, NR³R⁴, C₁-C₄ alkylcarboxyl, carboxyl, CR⁵R⁶—CO₂R⁷, C(O)NR³R⁴, C(O)NR³SO₂R³, NR³C(O)R³, C₃-C₆ cycloalkyl, 5- to 6-membered heterocyclic ring optionally substituted with R³, and heteroaryl optionally substituted with C₁-C₄ alkyl, hydroxyl, or oxo,
or R is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, and C₁-C₄ alkoxyl;
R¹ is hydrogen, hydroxyl, cyano, C₁-C₃ alkyl optionally substituted with one or more fluoro, C₃-C₄ cycloalkyl, or methoxyl optionally substituted with one to three fluoro;
R² is C₆-C₁₀ aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R² is C₁-C₄ haloalkoxyl, OR³, NR³R⁴, C(O)R³, or C(O)NR³R⁴;
each R³ is independently hydrogen, or C₃-C₆ cycloalkyl, C₄-C₆ heterocyclic, C₁-C₄ alkyl, each optionally substituted with one or more fluoro or carboxyl;
each R⁴ is independently R³, C(O) C₁-C₄ alkyl optionally substituted with one or more fluoro groups, C(O)NHC₁-C₄ alkyl optionally substituted with one or more fluoro groups, or C₁-C₄ alkyl optionally substituted with one or more deuterium, hydroxyl, fluoro, C₁-C₃ alkoxyl, NR³R³, carboxyl, or C(O)NR³R³;
or if R³ and R⁴ are attached to the same nitrogen atom, R³ and R⁴ together with their connecting nitrogen form a 4- to 6-membered heterocyclic ring optionally containing another heteroatom that is N, O, or S, and such heterocyclic ring is optionally substituted with one or more groups each independently selected from the group consisting of fluoro, chloro, cyano, carboxyl, C(O)NR³R³, NR³R⁴, NR¹C(O)R³, S(O)₂R³, S(O)₂NR³R³, C₁-C₄ alkylcarboxylate, and C₁-C₄ alkyl optionally substituted with hydroxyl, C₁-C₄ alkoxy, or NR¹C(O)R³;
R⁵ and R⁶ together with their connecting carbon form a 3- to 5-membered cyclic or heterocyclic ring; and
R⁷ is H or NH₂.

In certain embodiments, the present invention is directed to a compound, a prodrug thereof, or a pharmaceutically acceptable salt thereof, of formula (I):

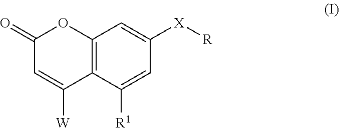

X is C(R³)(R³), C₃-C₆ cycloalkyl, C(R³)(R³)O, C(R³)(R³)OC(O), C(R³)—C(R³), N(R⁴), C(R³)(R³)N(R³), C(O)(NR³), or C(O);
W is C₆ aryl optionally substituted with one or more groups, each independently selected from the group consisting of fluoro, chloro, C₁ alkoxyl, and C₁ alkyl optionally substituted with one or more deuterium;
R is hydrogen, NR³R⁴, C(O)NR³R⁴, C₁-C₂ alkylcarboxyl, C(O)R³, carboxyl, hydroxyl, C₁ alkoxyl, NR³C(O)R³, C₆ aryl, or C₃-C₈ cycloalkyl optionally substituted with C(O)NR³R³,
or R is C₁-C₈alkyl optionally substituted with one or more groups, each independently selected from the group consisting of hydroxyl, cyano, C₁ alkoxyl, C₁ haloalkoxyl, phenyl, NR³R⁴, C₁-C₄ alkylcarboxyl, carboxyl, CR⁵R⁶—CO₂R⁷, C(O)NR³R⁴, C(O)NR³SO₂R³, NR³C(O)R³, C₃ cycloalkyl, 5- to 6-membered heterocyclic ring optionally substituted with R³, and heteroaryl optionally substituted with C₁-C₄ alkyl, hydroxyl, or oxo;
R¹ is hydrogen, hydroxyl, or C₁ alkyl;
each R³ is independently hydrogen, or C₆ cycloalkyl, C₆ heterocyclic, C₁-C₃ alkyl, each optionally substituted with carboxyl;
each R⁴ is independently R³, or C₁-C₃ alkyl optionally substituted with one or more deuterium, hydroxyl, fluoro, C₁ alkoxyl, NR³R³, carboxyl, or C(O)NR³R³;
or if R³ and R⁴ are attached to the same nitrogen atom, R³ and R⁴ together with their connecting nitrogen form a 4- to 6-membered heterocyclic ring optionally containing another heteroatom that is O, and such heterocyclic ring is optionally substituted with one or more groups each independently selected from the group consisting of cyano, carboxyl, C(O)NR³R³, NR³R⁴, NR¹C(O)R³, S(O)₂R³, S(O)₂NR³R³, C₁-C₂ alkylcarboxylate, and C₁ alkyl optionally substituted with hydroxyl, C₁ alkoxy, or NR¹C(O)R³;
R⁵ and R⁶ together with their connecting carbon form a 3- to 5-membered cyclic or heterocyclic ring; and
R⁷ is H or NH₂.

In certain embodiments, the present invention is directed to a compound, a prodrug thereof, or a pharmaceutically acceptable salt thereof, of formula (I):

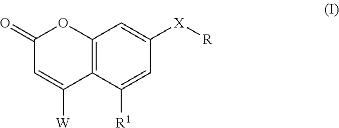

wherein:
X is C(R³)(R³), C₃ cycloalkyl, C(R³)═C(R³), N(R⁴), or (R³)(R³)N(R³);
W is C₆ aryl optionally substituted with one or more groups, each independently selected from the group consisting of fluoro, chloro, and C₁ alkyl;
R is C(O)NR³R⁴, C₁-C₂ alkylcarboxyl, C(O)R³, carboxyl, or C₁ alkoxyl,
or R is C₁-C₄ alkyl optionally substituted with one or more groups, each independently selected from the group consisting of C₁ alkoxyl, phenyl, C₁-C₄ alkylcarboxyl, carboxyl, C(O)NR³R⁴, C₃ cycloalkyl, 5-membered heterocyclic, and heteroaryl optionally substituted with C₁ alkyl, hydroxyl, or oxo;
each R³ is independently hydrogen, or C₆ cycloalkyl, C₆ heterocyclic, C₁-C₃ alkyl, each optionally substituted with carboxyl;
each R⁴ is independently R³, or C₁-C₂ alkyl optionally substituted with one or more hydroxyl, fluoro, C₁ alkoxyl, or C(O)NR³R³,
or if R³ and R⁴ are attached to the same nitrogen atom, R³ and R⁴ together with their connecting nitrogen form a 5- to 6-membered heterocyclic ring optionally containing another heteroatom that is O, and such heterocyclic ring is optionally substituted with one or more groups each independently selected from the group consisting of cyano, carboxyl, C(O)NR³R³, S(O)₂R³, and S(O)₂NR³R³.

In certain embodiments, X is NR⁴.

In certain embodiments, X is NH.

In certain embodiments, X is NCH₃.

In certain embodiments, X is C(R³)(R³).

In certain embodiments, X is CHR₃.

In certain embodiments, X is CH₂.

In certain embodiments, X is C(R³)═C(R³).

In certain embodiments, X is C(H)═C(CH₃).

In certain embodiments, W is phenyl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl, provided that at least one substituent is at an ortho position relative to the attachment point with the central ring.

In certain embodiments, W is phenyl.

In certain embodiments, W is 2-chlorophenyl.

In certain embodiments, W is 4-fluorophenyl.

In certain embodiments, W is 2-methoxyphenyl.

In certain embodiments, W is 2-chloro-4-fluorophenyl.

In certain embodiments, W is 2-methyl-phenyl.

In certain embodiments, R¹ is hydrogen.

In certain embodiments, R¹ is methyl.

In certain embodiments, R¹ is hydroxyl.

In certain embodiments, R is hydrogen.

In certain embodiments, R is C₁-C₆ alkyl.

In certain embodiments, R is methyl.

In certain embodiments, R is n-propyl.

In certain embodiments, R is iso-propyl.

In certain embodiments, R is C₁-C₄ alkylcarboxyl.

In certain embodiments, R is ethyl carboxyl.

In certain embodiments, R is carboxyl.

In certain embodiments, R is CH₂CH₂R².

In certain embodiments, R² is phenyl.

In certain embodiments, R² is OR¹.

In certain embodiments, R is CH₂CH₂OCH₃.

In certain embodiments, R is CH₂CH₂OH.

In certain embodiments, R² is C(O)NR²R³.

In certain embodiments, R² is C(O)NHCH₃.

In certain embodiments, R² is C(O)NHCH₂CH₂CH₃.

In certain embodiments, R² is C(O)N(CH₃)CH₂CH₃.

In certain embodiments, the compound is (R)-2-((2-oxo-4-(o-tolyl)-2H-chromen-7-yl)amino)-3-(trifluoromethoxy)propanamide, Example 252, or a pharmaceutically acceptable salt thereof:

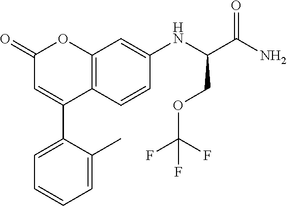 [SMILES: Cc1ccccc1-c1cc(=O)oc2cc(N[C@H](COC(F)(F)F)C(N)=O)ccc12]

In certain embodiments, the compound is (S)-2-((2-oxo-4-(o-tolyl)-2H-chromen-7-yl)amino)-3-(trifluoromethoxy)propanamide, Example 253, or a pharmaceutically acceptable salt thereof:

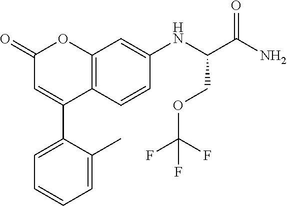 [SMILES: Cc1ccccc1-c1cc(=O)oc2cc(N[C@@H](COC(F)(F)F)C(N)=O)ccc12]

In certain embodiments, the compound is (S)-2-(methyl(2-oxo-4-(o-tolyl)-2H-chromen-7-yl)amino)-3-(trifluoromethoxy)propanamide, Example 254, or a pharmaceutically acceptable salt thereof:

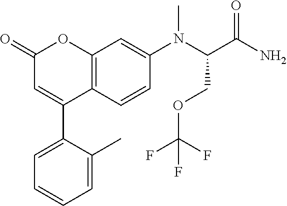 [SMILES: Cc1ccccc1-c1cc(=O)oc2cc(N(C)[C@@H](COC(F)(F)F)C(N)=O)ccc12]

In certain embodiments, the compound is (R)-2-(methyl(2-oxo-4-(o-tolyl)-2H-chromen-7-yl)amino)-3-(trifluoromethoxy)propanamide, Example 255, or a

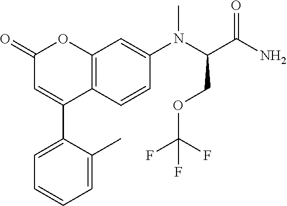 [SMILES: Cc1ccccc1-c1cc(=O)oc2cc(N(C)[C@H](COC(F)(F)F)C(N)=O)ccc12]

In certain embodiments, the compound is (1R,2S)-2-(2-oxo-4-(o-tolyl)-2H-chromen-7-yl)cyclopropane-1-carboxylic acid, Example 256, or a pharmaceutically acceptable salt thereof:

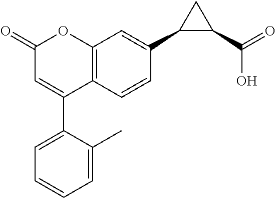 [SMILES: Cc1ccccc1-c1cc(=O)oc2cc([C@H]3C[C@H]3C(=O)O)ccc12]

In certain embodiments, the compound is (1S,2R)-2-(2-oxo-4-(o-tolyl)-2H-chromen-7-yl)cyclopropane-1-carboxylic acid, Example 257, or a pharmaceutically acceptable salt thereof:

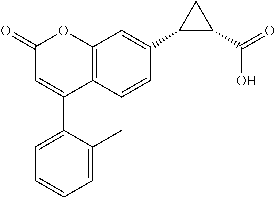 [SMILES: Cc1ccccc1-c1cc(=O)oc2cc([C@@H]3C[C@@H]3C(=O)O)ccc12]

In certain embodiments, the compound is N-methyl-2-((methyl-d₃) (2-oxo-4-(o-tolyl)-2H-chromen-7-yl)amino)acetamide, Example 258, or a pharmaceutically

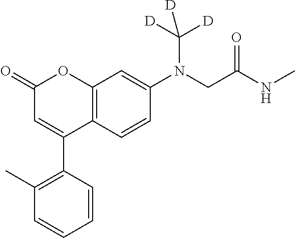 [SMILES: [2H]C([2H])([2H])N(CC(=O)NC)c1ccc2c(-c3ccccc3C)cc(=O)oc2c1]

In certain embodiments, the compound is N-methyl-2-(methyl(4-(2-(methyl-d₃)phenyl)-2-oxo-2H-chromen-7-yl)amino)acetamide, Example 259, or a pharmaceutically acceptable salt thereof:

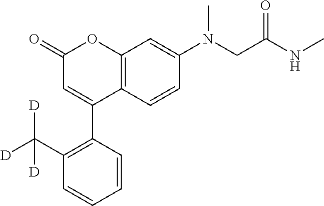 [SMILES: [2H]C([2H])([2H])c1ccccc1-c1cc(=O)oc2cc(N(C)CC(=O)NC)ccc12]

In certain embodiments, the compound is N-(methyl-d₃)-2-((methyl-d₃) (2-oxo-4-(o-tolyl)-2H-chromen-7-yl)amino)acetamide, Example 260, or a pharmaceutically acceptable salt thereof:

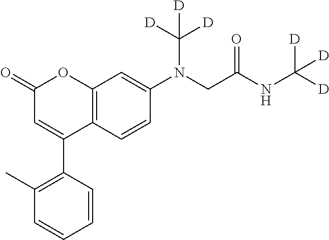 [SMILES: [2H]C([2H])([2H])NC(=O)CN(c1ccc2c(-c3ccccc3C)cc(=O)oc2c1)C([2H])([2H])[2H]]

In certain embodiments, the compound is N-(methyl-d₃)-2-((methyl-d₃) (4-(2-(methyl-d₃)phenyl)-2-oxo-2H-chromen-7-yl)amino)acetamide, Example 261, or a

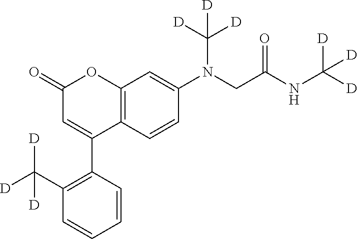 [SMILES: [2H]C([2H])([2H])NC(=O)CN(c1ccc2c(-c3ccccc3C([2H])([2H])[2H])cc(=O)oc2c1)C([2H])([2H])[2H]]

In certain embodiments, the compound is N-methyl-N—(N-methyl-N-(2-oxo-4-(o-tolyl)-2H-chromen-7-yl)glycyl)glycine, Example 262, or a pharmaceutically acceptable salt thereof:

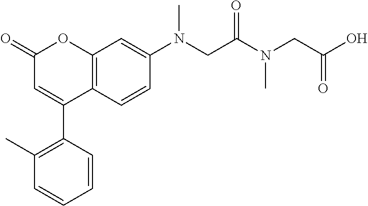 [SMILES: Cc1ccccc1-c1cc(=O)oc2cc(N(C)CC(=O)N(C)CC(=O)O)ccc12]

In certain embodiments, the compound is N-(2-hydroxyethyl)-N-methyl-2-(methyl(2-oxo-4-(o-tolyl)-2H-chromen-7-yl)amino)acetamide, Example 263, or a pharmaceutically acceptable salt thereof:

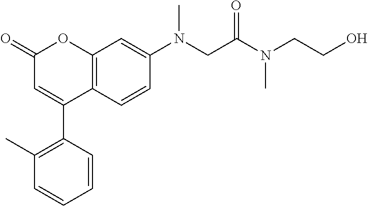 [SMILES: Cc1ccccc1-c1cc(=O)oc2cc(N(C)CC(=O)N(C)CCO)ccc12]

In certain embodiments, the compound is (S)—N-(2-hydroxypropyl)-2-(methyl(2-oxo-4-(o-tolyl)-2H-chromen-7-yl)amino)acetamide, Example 264, or a

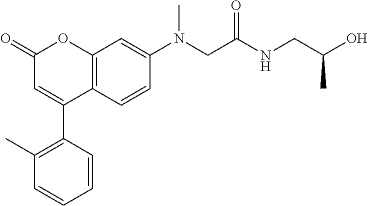 [SMILES: Cc1ccccc1-c1cc(=O)oc2cc(N(C)CC(=O)NC[C@H](C)O)ccc12]

In certain embodiments, the compound is (R)—N-(2-hydroxypropyl)-2-(methyl(2-oxo-4-(o-tolyl)-2H-chromen-7-yl)amino)acetamide, Example 265, or a pharmaceutically acceptable salt thereof:

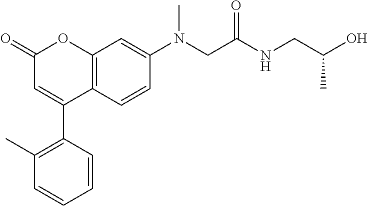 [SMILES: Cc1ccccc1-c1cc(=O)oc2cc(N(C)CC(=O)NC[C@@H](C)O)ccc12]

In certain embodiments, the compound is (S)—N-(1-hydroxypropan-2-yl)-2-(methyl(2-oxo-4-(o-tolyl)-2H-chromen-7-yl)amino)acetamide, Example 266, or a pharmaceutically acceptable salt thereof:

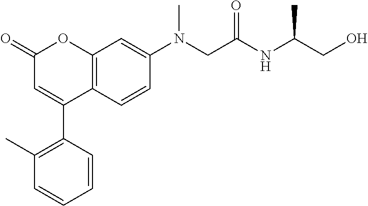 [SMILES: Cc1ccccc1-c1cc(=O)oc2cc(N(C)CC(=O)N[C@@H](C)CO)ccc12]

In certain embodiments, the compound is (R)—N-(1-hydroxypropan-2-yl)-2-(methyl(2-oxo-4-(o-tolyl)-2H-chromen-7-yl)amino)acetamide, Example 267, or a pharmaceutically acceptable salt thereof:

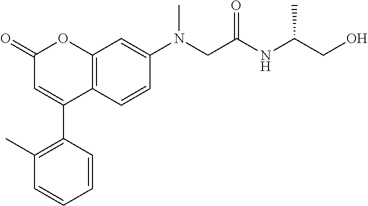 [SMILES: Cc1ccccc1-c1cc(=O)oc2cc(N(C)CC(=O)N[C@H](C)CO)ccc12]

In certain embodiments, the compound is (R)-2-(2-(methyl(2-oxo-4-(o-tolyl)-2H-chromen-7-yl)amino)acetamido)propanamide, Example 268, or a pharmaceutically

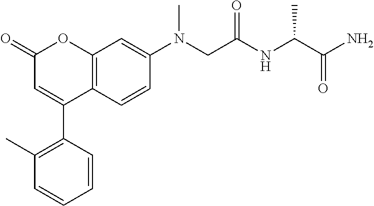 [SMILES: Cc1ccccc1-c1cc(=O)oc2cc(N(C)CC(=O)N[C@H](C)C(N)=O)ccc12]

In certain embodiments, the compound is (S)-2-(2-(methyl(2-oxo-4-(o-tolyl)-2H-chromen-7-yl)amino)acetamido)propanamide, Example 269, or a pharmaceutically acceptable salt thereof:

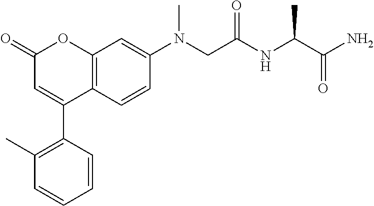 [SMILES: Cc1ccccc1-c1cc(=O)oc2cc(N(C)CC(=O)N[C@@H](C)C(N)=O)ccc12]

In certain embodiments, the compound is (S)—N-(2-amino-2-oxoethyl)-2-(methyl(2-oxo-4-(o-tolyl)-2H-chromen-7-yl)amino)propanamide, Example 270, or a pharmaceutically acceptable salt thereof:

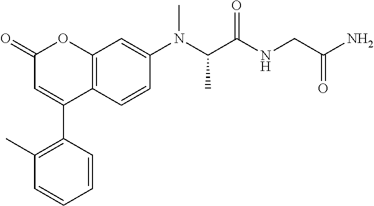 [SMILES: Cc1ccccc1-c1cc(=O)oc2cc(N(C)[C@@H](C)C(=O)NCC(N)=O)ccc12]

In certain embodiments, the compound is (R)—N-(2-amino-2-oxoethyl)-2-(methyl(2-oxo-4-(o-tolyl)-2H-chromen-7-yl)amino)propanamide, Example 271, or a

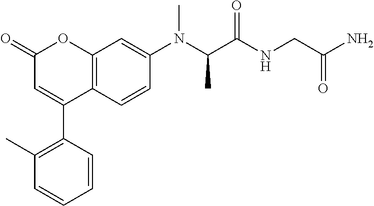 [SMILES: Cc1ccccc1-c1cc(=O)oc2cc(N(C)[C@H](C)C(=O)NCC(N)=O)ccc12]

In certain embodiments, the compound is 2-(methyl(2-oxo-4-(o-tolyl)-2H-chromen-7-yl)amino)-N-(oxazol-2-ylmethyl)acetamide, Example 272, or a pharmaceutically acceptable salt thereof:

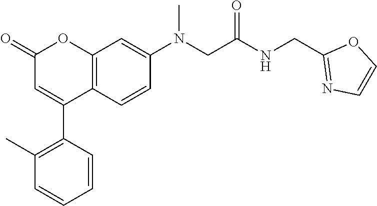 [SMILES: Cc1ccccc1-c1cc(=O)oc2cc(N(C)CC(=O)NCc3ncco3)ccc12]

In certain embodiments, the compound is 1-((methyl(2-oxo-4-(o-tolyl)-2H-chromen-7-yl)amino)methyl)cyclobutane-1-carboxylic acid, Example 273, or a pharmaceutically acceptable salt thereof:

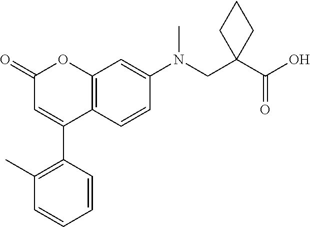 [SMILES: Cc1ccccc1-c1cc(=O)oc2cc(N(C)CC3(C(=O)O)CCC3)ccc12]

In certain embodiments, the compound is 3-((methyl(2-oxo-4-(o-tolyl)-2H-chromen-7-yl)amino)methyl) oxetane-3-carboxylic acid, Example 274, or a pharmaceutically acceptable salt thereof:

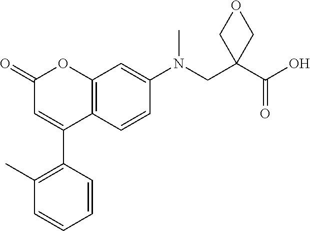 [SMILES: Cc1ccccc1-c1cc(=O)oc2cc(N(C)CC3(C(=O)O)COC3)ccc12]

In certain embodiments, the compound is (S)-3-methoxy-2-methyl-2-((methyl(2-oxo-4-(o-tolyl)-2H-chromen-7-yl)amino)methyl)propanoic acid, Example 275, or a pharmaceutically acceptable salt thereof:

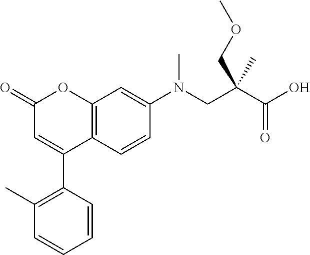 [SMILES: COC[C@](C)(CN(C)c1ccc2c(-c3ccccc3C)cc(=O)oc2c1)C(=O)O]

In certain embodiments, the compound is (R)-3-methoxy-2-methyl-2-((methyl(2-oxo-4-(o-tolyl)-2H-chromen-7-yl)amino)methyl)propanoic acid, Example 276, or a pharmaceutically acceptable salt thereof:

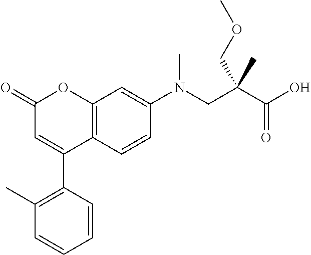 [SMILES: COC[C@@](C)(CN(C)c1ccc2c(-c3ccccc3C)cc(=O)oc2c1)C(=O)O]

In certain embodiments, the compound is 3-methyl-3-(methyl(2-oxo-4-(o-tolyl)-2H-chromen-7-yl)amino)butanoic acid, Example 277, or a pharmaceutically acceptable salt thereof:

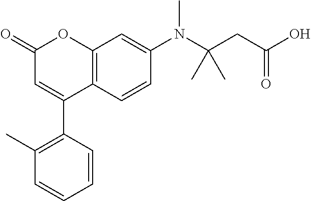 [SMILES: Cc1ccccc1-c1cc(=O)oc2cc(N(C)C(C)(C)CC(=O)O)ccc12]

In certain embodiments, the compound is N-methyl-N-(2-oxo-4-(o-tolyl)-2H-chromen-7-yl)glycyl-D-alanine, Example 278, or a pharmaceutically acceptable salt thereof:

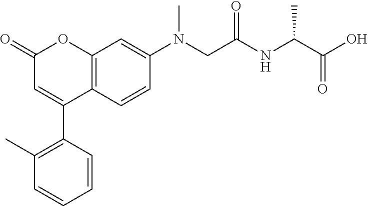 [SMILES: Cc1ccccc1-c1cc(=O)oc2cc(N(C)CC(=O)N[C@H](C)C(=O)O)ccc12]

In certain embodiments, the compound is N-methyl-N-(2-oxo-4-(o-tolyl)-2H-chromen-7-yl)glycyl-L-alanine, Example 279, or a pharmaceutically acceptable salt thereof:

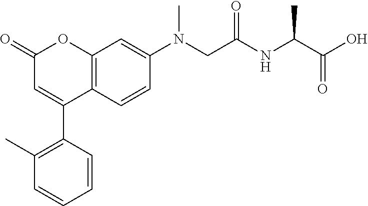 [SMILES: Cc1ccccc1-c1cc(=O)oc2cc(N(C)CC(=O)N[C@@H](C)C(=O)O)ccc12]

In certain embodiments, the compound is 2-methyl-2-(2-(methyl(2-oxo-4-(o-tolyl)-2H-chromen-7-yl)amino)acetamido)propanoic acid, Example 280, or a pharmaceutically acceptable salt thereof:

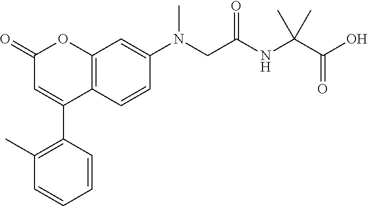 [SMILES: Cc1ccccc1-c1cc(=O)oc2cc(N(C)CC(=O)NC(C)(C)C(=O)O)ccc12]

In certain embodiments, the compound is N-(2-hydroxyethyl)-2-((methyl-d₃) (2-oxo-4-(o-tolyl)-2H-chromen-7-yl)amino)acetamide, Example 281, or a

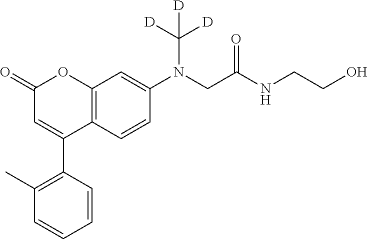 [SMILES: [2H]C([2H])([2H])N(CC(=O)NCCO)c1ccc2c(-c3ccccc3C)cc(=O)oc2c1]

In certain embodiments, the compound is N-(2-hydroxyethyl)-2-((methyl-d₃) (4-(2-(methyl-d₃)phenyl)-2-oxo-2H-chromen-7-yl)amino)acetamide, Example 282, or a pharmaceutically acceptable salt thereof:

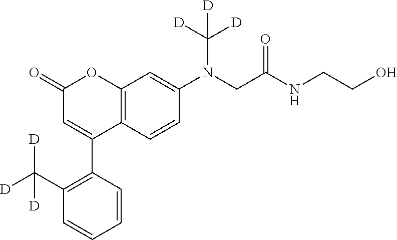 [SMILES: [2H]C([2H])([2H])c1ccccc1-c1cc(=O)oc2cc(N(CC(=O)NCCO)C([2H])([2H])[2H])ccc12]

In certain embodiments, the compound is N-(2-hydroxyethyl)-2-(methyl(4-(2-(methyl-d₃)phenyl)-2-oxo-2H-chromen-7-yl)amino)acetamide, Example 283, or a pharmaceutically acceptable salt thereof:

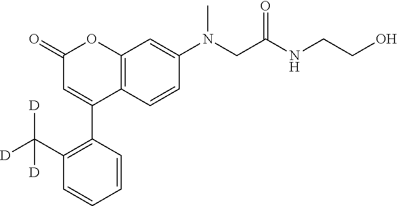 [SMILES: [2H]C([2H])([2H])c1ccccc1-c1cc(=O)oc2cc(N(C)CC(=O)NCCO)ccc12]

In certain embodiments, the compound is 2-((4-(5-fluoro-2-methylphenyl)-2-oxo-2H-chromen-7-yl)(methyl)amino)-N-(2-hydroxyethyl)acetamide, Example 284, or a pharmaceutically acceptable salt thereof:

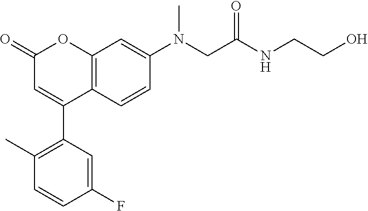 [SMILES: Cc1ccc(F)cc1-c1cc(=O)oc2cc(N(C)CC(=O)NCCO)ccc12]

In certain embodiments, the compound is 2-((4-(3-fluoro-2-methylphenyl)-2-oxo-2H-chromen-7-yl)(methyl)amino)-N-(2-hydroxyethyl)acetamide, Example 285, or a

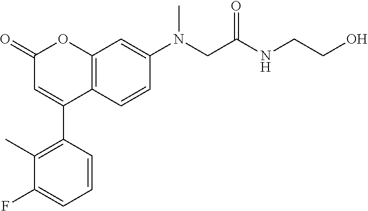 [SMILES: Cc1c(F)cccc1-c1cc(=O)oc2cc(N(C)CC(=O)NCCO)ccc12]

In certain embodiments, the compound is 2-((4-(4-fluoro-2-methylphenyl)-2-oxo-2H-chromen-7-yl)(methyl)amino)-N-(2-hydroxyethyl)acetamide, Example 286, or a pharmaceutically acceptable salt thereof:

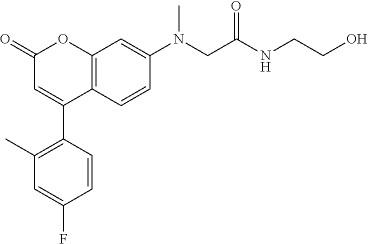 [SMILES: Cc1cc(F)ccc1-c1cc(=O)oc2cc(N(C)CC(=O)NCCO)ccc12]

In certain embodiments, the compound is 2,2-dimethyl-3-(methyl(4-(2-(methyl-d₃)phenyl)-2-oxo-2H-chromen-7-yl)amino)propanoic acid, Example 287, or a pharmaceutically acceptable salt thereof:

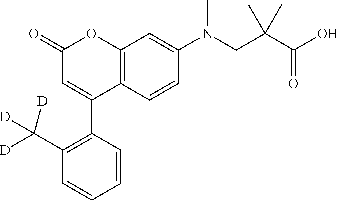 [SMILES: [2H]C([2H])([2H])c1ccccc1-c1cc(=O)oc2cc(N(C)CC(C)(C)C(=O)O)ccc12]

In certain embodiments, the compound is 2,2-dimethyl-3-((methyl-d₃) (2-oxo-4-(o-tolyl)-2H-chromen-7-yl)amino)propanoic acid, Example 288, or a pharmaceutically

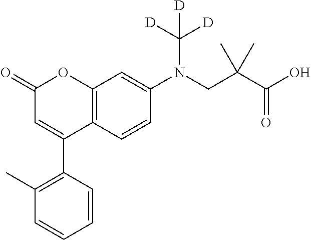 [SMILES: [2H]C([2H])([2H])N(CC(C)(C)C(=O)O)c1ccc2c(-c3ccccc3C)cc(=O)oc2c1]

In certain embodiments, the compound is 2,2-dimethyl-3-((methyl-d₃) (4-(2-(methyl-d₃)phenyl)-2-oxo-2H-chromen-7-yl)amino)propanoic acid, Example 289, or a pharmaceutically acceptable salt thereof:

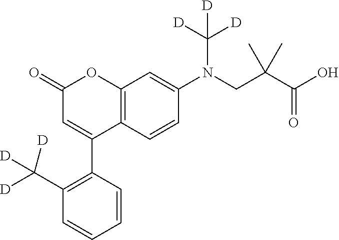 [SMILES: [2H]C([2H])([2H])c1ccccc1-c1cc(=O)oc2cc(N(CC(C)(C)C(=O)O)C([2H])([2H])[2H])ccc12]

In certain embodiments, the compound is 3-((methyl-d₃) (2-oxo-4-(o-tolyl)-2H-chromen-7-yl)amino)propanoic acid, Example 290, or a pharmaceutically acceptable salt thereof:

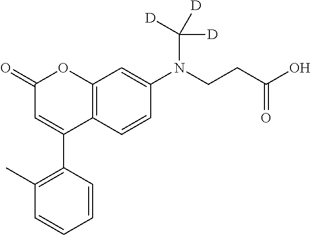 [SMILES: [2H]C([2H])([2H])N(CCC(=O)O)c1ccc2c(-c3ccccc3C)cc(=O)oc2c1]

In certain embodiments, the compound is 3-((methyl-d₃) (4-(2-(methyl-d₃)phenyl)-2-oxo-2H-chromen-7-yl)amino)propanoic acid, Example 291, or a

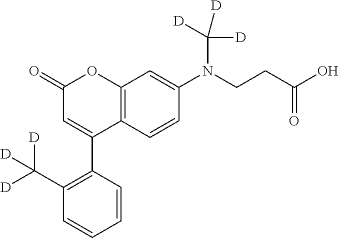 [SMILES: [2H]C([2H])([2H])c1ccccc1-c1cc(=O)oc2cc(N(CCC(=O)O)C([2H])([2H])[2H])ccc12]

In certain embodiments, the compound is 3-((4-(2-(difluoromethyl)phenyl)-2-oxo-2H-chromen-7-yl)(methyl)amino)propanoic acid, Example 292, or a pharmaceutically acceptable salt thereof:

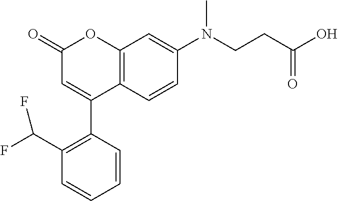 [SMILES: CN(CCC(=O)O)c1ccc2c(-c3ccccc3C(F)F)cc(=O)oc2c1]

In certain embodiments, the compound is 3-(methyl(2-oxo-4-(2-(trifluoromethyl)phenyl)-2H-chromen-7-yl)amino)propanoic acid, Example 293, or a pharmaceutically acceptable salt thereof:

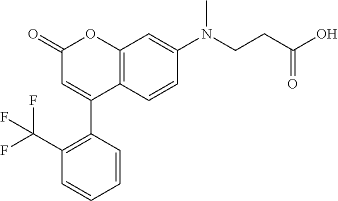 [SMILES: CN(CCC(=O)O)c1ccc2c(-c3ccccc3C(F)(F)F)cc(=O)oc2c1]

In certain embodiments, the compound is 3-((4-(2-cyanophenyl)-2-oxo-2H-chromen-7-yl)(methyl)amino)propanoic acid, Example 294, or a pharmaceutically

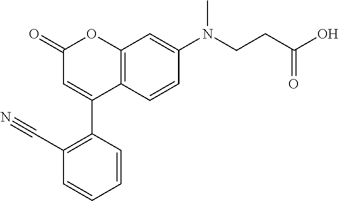 [SMILES: CN(CCC(=O)O)c1ccc2c(-c3ccccc3C#N)cc(=O)oc2c1]

In certain embodiments, the compound is 7-((3-fluorophenethyl)(methyl)amino)-4-(o-tolyl)-2H-chromen-2-one, Example 295, or a pharmaceutically acceptable salt thereof:

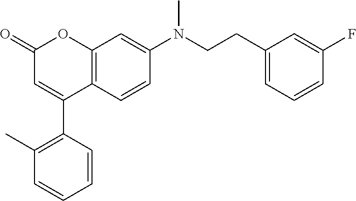 [SMILES: Cc1ccccc1-c1cc(=O)oc2cc(N(C)CCc3cccc(F)c3)ccc12]

In certain embodiments, the compound is 4-(o-tolyl)-7-((trifluoromethoxy)methyl)-2H-chromen-2-one, Example 296, or a pharmaceutically acceptable salt thereof:

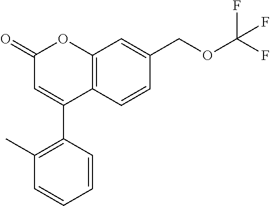 [SMILES: Cc1ccccc1-c1cc(=O)oc2cc(COC(F)(F)F)ccc12]

In certain embodiments, the compound is 7-((4-fluorophenyl)(methyl)amino)-4-(o-tolyl)-2H-chromen-2-one, Example 297, or a pharmaceutically acceptable salt thereof:

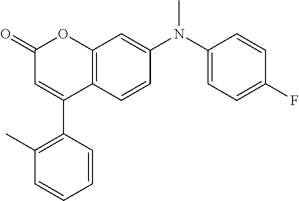 [SMILES: Cc1ccccc1-c1cc(=O)oc2cc(N(C)c3ccc(F)cc3)ccc12]

In certain embodiments, the compound is 4-(2-(methyl-d₃)phenyl)-7-((trifluoromethoxy)methyl)-2H-chromen-2-one, Example 298, or a pharmaceutically

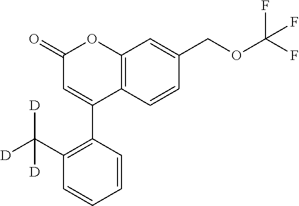 [SMILES: [2H]C([2H])([2H])c1ccccc1-c1cc(=O)oc2cc(COC(F)(F)F)ccc12]

In certain embodiments, the compound is 4-(2-(methyl-d₃)phenyl)-7-((2,2,2-trifluoroethoxy)methyl)-2H-chromen-2-one, Example 299, or a pharmaceutically acceptable salt thereof:

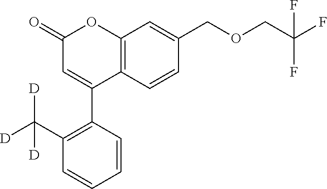 [SMILES: [2H]C([2H])([2H])c1ccccc1-c1cc(=O)oc2cc(COCC(F)(F)F)ccc12]

In certain embodiments, the compound is 4-(o-tolyl)-7-((2,2,2-trifluoroethoxy)methyl)-2H-chromen-2-one, Example 300, or a pharmaceutically acceptable salt thereof:

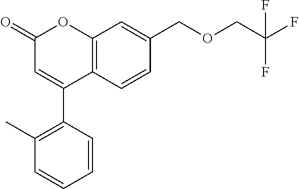 [SMILES: Cc1ccccc1-c1cc(=O)oc2cc(COCC(F)(F)F)ccc12]

The compounds of the present invention may contain asymmetric carbon atoms (sometimes as the result of a deuterium atom) and thereby can exist as either individual stereoisomers or mixtures of the enantiomers or mixtures of diastereomers. Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. Carbon atoms labelled with * or ** refer to a compound that is chiral but the absolute stereochemistry has not been determined.

The compounds of the present invention may contain double bonds that may exist in more than one geometric isomer. Examples of such double bonds are carbon-carbon double bonds which form alkenes. In the case of carbon-carbon double bonds, the geometric isomers may be E or Z isomers.

Unless otherwise indicated, when a disclosed compound is named or depicted by a structure without specifying the geometric isomerism and has one or more possible geometric isomers, it is understood to represent all possible geometric isomers of the compound.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and ketoenol tautomerism is possible, it is understood that the substituent might in fact be present, in whole or in part, in the oxo (═O) form.

Compounds of the present invention may exist in amorphous form and/or one or more crystalline forms. As such all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the invention. See, e.g., Gannes, L Z et al., Comp Biochem Physiol Mol Integr Physiol, 119, 725 (1998). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium. See Pirali et al, J. Med. Chem. 62, 5276-97 (2019). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances deuteration may have little to no impact on these properties, or may affect them in an undesirable manner. Whether and/or how such replacement will impact drug properties can only be determined if the drug is synthesized, evaluated, and compared to its non-deuterated counterpart. See Fukuto et al., J. Med. Chem. 34, 2871-76 (1991). Because some drugs have multiple sites of metabolism or more than one active sites for binding to a target, it is unpredictable as to which sites may benefit by deuterium replacement or to what extent isotope enrichment is necessary to produce a beneficial effect.

### Preparation of the Compounds

The starting materials and reagents used in each step in the preparation are known and can be readily prepared or purchased from commercial sources.

The compound obtained in each step can also be used for the next reaction as a reaction mixture thereof or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min. to 48 h., preferably 10 min. to 8 h.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water.

Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schützenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like. 0152 In each step, protection or deprotection of a functional group is performed by a known method, for example, the methods described in “Protective Groups in Organic Synthesis, 4^{th} Ed.” (Theodora W. Greene, Peter G. M. Wuts) Wiley-Interscience, 2007; “Protecting Groups 3rd Ed.” (P. J. Kocienski) Thieme, 2004 and the like.

Deuterated POLRMT modulators of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (“NMR”) can be used to determine a compound's structure while mass spectroscopy (“MS”) can be used to determine the amount of deuterium atom in the compound by comparison to its non-deuterated form.

### Compositions

The present invention further includes pharmaceutical compositions of the compounds or a pharmaceutically acceptable salt of said compounds. In addition to the compound of the invention or a salt thereof, the pharmaceutical compositions comprise one or more pharmaceutically acceptable excipients, such excipients being compatible with other ingredients in the composition and also being not toxic or otherwise harmful. Examples of excipients include carriers, lubricants, binders, disintegrants, solvents, solubilizing agents, suspending agents, isotonic agents, buffers, soothing agents, preservatives, antioxidants, colorants, taste-modifying agents, absorbents, and/or wetting agents.

The pharmaceutical compositions of the invention include those suitable for oral, rectal, nasal, topical, buccal, sublingual, vaginal or parenteral (including subcutaneous, intramuscular, intravenous and intradermal) administration. Such compositions may be prepared by any methods well known in the art of pharmaceutical formulations and pharmacy. See, e.g., Remington: The Science and Practice of Pharmacy, Elsevier Science, 23rd ed. (2020).

Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, hydrogels, gelatin, hydrogenated naphthalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions can be administered by oral ingestion. Compositions intended for oral use can be prepared in solid or liquid forms, according to any method known to a person of ordinary skill in the art for the manufacture of pharmaceutical compositions. Solid dosage forms for oral administration include capsules (both soft and hard gelatin capsules), tablets, powders, and granules. Generally, these pharmaceutical preparations contain active ingredients admixed with pharmaceutically acceptable excipients. These excipients include, for example, inert diluents, such as calcium carbonate, sodium carbonate, lactose, sucrose, glucose, mannitol, cellulose, starch, calcium phosphate, sodium phosphate, kaolin and the like; binding agents, buffering agents, and/or lubricating agents (e.g., magnesium stearate) may also be used. Tablets and capsules can additionally be prepared with release-controlling coatings such as enteric coatings. The compositions may optionally contain sweetening, flavoring, coloring, perfuming, and preserving agents in order to provide a more palatable preparation.

In another embodiment, a pharmaceutical composition of this invention further comprises a second therapeutic agent. The second therapeutic agent may be selected from any pharmaceutically active compound; preferably the second therapeutic agent is known to correct mitochondrial dysfunction. Alternatively, the compounds of the invention and second therapeutic agent may be administered together (within less than 24 hours of one another, consecutively or simultaneously) but in separate pharmaceutical compositions. In certain embodiments, the compounds on the invention and second therapeutic agent can be administered separately (e.g., more than 24 hours of one another.) If the second therapeutic agent acts synergistically with the compounds of this invention, the therapeutically effective amount of such compounds and/or the second therapeutic agent may be less that such amount required when either is administered alone.

For the treatment of cancer, the compounds described herein may be administered in combination with a chemotherapeutic agent. Therapeutically effective amounts of the additional chemotherapeutic agent(s) are well known to those skilled in the art. However, it is well within the attending physician to determine the amount of other chemotherapeutic agent(s) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting.

### EXAMPLES

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by mass spectrometry and/or NMR, where peaks assigned to the characteristic protons in the title compound are presented where appropriate. ¹H NMR shift (δ) are given in parts per million (ppm) down field from an internal reference standard.

The abbreviations used herein are known to a person of ordinary skill in the art. A partial list of abbreviations that may be used herein include: acetonitrile (MeCN), ammonium carbonate (NH₄)₂CO₃, ammonium chloride (NH₄+Cl), aqueous (aq.), 1,1′-bis(diphenylphosphino) ferrocene (dppf), 1,3-bis(diphenylphosphino)propane (dppp), bis(pinacolato)diboron (B₂pin₂), N-bromosuccinimide (NBS), bromo-tris-pyrrolidino-phosphonium hexafluorophosphate (PyBroP), boron tribromide (BBr₃), butyl lithium (BuLi), calculated (Calcd.), cesium carbonate (Cs₂CO₃), dichloromethane (DCM, CH₂Cl₂), N,N-dicyclohexylcarbodiimide (DCC), dichloroethane (DCE), diethyl azodicarboxylate (DEAD), diisopropyl azodicarboxylate (DIAD), N,N-diisopropylethylamine (DIPEA), 4-dimethylaminopyridine (DMAP), dimethylformamide (DMF), dimethyl sulfoxide (DMSO), di-tert-butyl decarbonate (Boc₂O), 1-ethyl-3-(3-dimethylaminopropyl) carbodiimide (EDC), electrospray ionization (ESI), enantiomeric excess (ee), ethyl acetate (EtOAc), hour (h.), N-[(dimethylamino)-1H-1,2,3-triazolo-[4,5-b]pyridin-1-ylmethylene]-N-methylmethanaminium hexafluorophosphate N-oxide (HATU), high performance liquid chromatography (HPLC), hydroxybenzotriazole (HOBt), isopropyl alcohol (IPA), lithium hydroxide monohydrate (LiOH·H2O), methanol (MeOH), methyl iodide (Mel), minutes (min.), potassium carbonate (K₂CO₃), liquid chromatography-mass spectrometry (LCMS), phenyliodide (III) diacetate (PIDA), propylphosphonic anhydride (T₃P), reverse phase (RP), room/ambient temperature (rt, RT), silver oxide (Ag₂O), sodium hydride (NaH), sodium sulfate (Na₂SO₃), supercritical fluid chromatography (SFC), tetrahydrofuran (THF), triethylamine (Et₃N), thionyl chloride (SOCl₂), triphenylphosphine (PPh₃), dicyclohexyl[2′,4′,6′-tris(propan-2-yl) [1,1′-biphenyl]-2-yl]phosphane (XPhos).

The invention will now be described in reference to the following examples. These examples are not to be regarded as limiting the scope of the present invention, but shall only serve in an illustrative manner. Table 1 provides a listing of the example compounds of the present invention and IC₅₀ values.

### Example 1: Synthesis of 7-(dimethylamino)-4-(o-tolyl)-2H-chromen-2-one

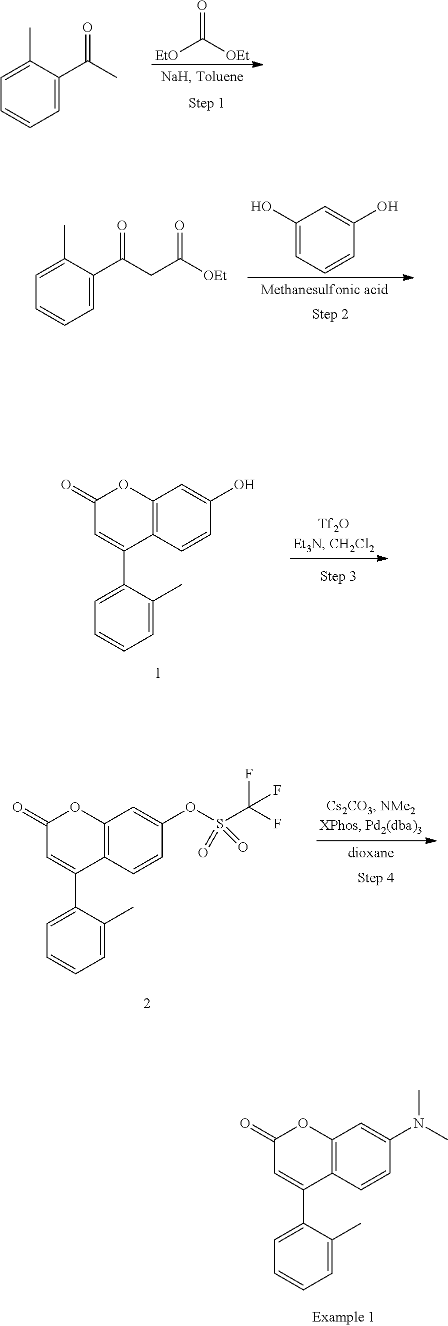

Synthesis of ethyl 3-oxo-3-(o-tolyl)propanoate [Step 1]: To a suspension of NaH (1.28 g, 53.6 mmol) in toluene (30 mL) at ambient temperature was added diethyl carbonate (7.2 mL, 59.62 mmol). The reaction mixture was stirred for 15 min., then 2-methylacetophenone (2 g, 14.9 mmol) was added. The reaction mixture was then gradually warmed to 100° C. and stirred for 4 h. The reaction mixture was then cooled to 0-1° C. and quenched with saturated aq. NH₄Cl solution and extracted with EtOAc three times. The organic layers were combined, washed with water, brine, dried over anhydrous sodium sulfate, filtered, and concentrated under reduced pressure. The product was purified by flash column chromatography on silica gel to give ethyl 3-oxo-3-(o-tolyl)propanoate (2.5 g). LCMS (ESI): Calcd. for C₁₂H₁₄O₃: 206, found [M+H]⁺=207.

Synthesis of 7-hydroxy-4-(o-tolyl)-2H-chromen-2-one, 1 [Step 2]: Methane sulfonic acid (2 mL) was added dropwise to a mixture of ethyl 3-oxo-3-(o-tolyl)propanoate (1 g, 4.84 mmol) and resorcinol (0.53 g, 4.84 mmol) at ambient temperature. The reaction mixture was stirred for 16 h. The reaction mixture was cooled to 0-1° C. and quenched with water. The product was filtered, washed with water, pentane, and dried under reduced pressure to afford 7-hydroxy-4-(o-tolyl)-2H-chromen-2-one (1, 1 g). ¹H NMR (400 MHz, DMSO-d₆) δ 10.63 (s, 1H), 7.43-7.32 (m, 3H), 7.23 (d, 1H), 6.81-6.71 (m, 3H), 6.09 (s, 1H), 2.16 (s, 3H). LCMS (ESI): Calcd. for C₁₆H₁₂O₃: 252, found [M+H]⁺=253.

Synthesis of 2-oxo-4-(o-tolyl)-2H-chromen-7-yl trifluoromethanesulfonate, 2 [Step 1]: To a stirred solution of 7-hydroxy-4-(o-tolyl) chromen-2-one (1, 300 mg, 1.19 mmol) in CH₂Cl₂ (10 mL) at 0° C. was added Et₃N (0.20 mL, 1.43 mmol) followed by dropwise addition of triflic anhydride (0.30 mL, 1.78 mmol). The reaction mixture was gradually warmed to ambient temperature, then stirred for 4 h. The reaction was monitored by LCMS analysis until completion. The reaction mixture was then diluted with CH₂Cl₂, washed with saturated aqueous NaHCO₃ solution, brine, dried over anhydrous sodium sulfate, filtered, then concentrated under reduced pressure. The product was purified by flash column chromatography on silica gel to afford [4-(o-tolyl)-2-oxo-chromen-7-yl]trifluoromethanesulfonate (2, 350 mg). LCMS (ESI) Calcd. for C₁₇H₁₁F₃O₅S: 384, found: [M+H]⁺=385. ¹H NMR (400 MHz, DMSO-d₆) δ 7.87 (d, 1H), 7.51-7.32 (m, 4H), 7.28 (d, 1H), 7.14 (d, 1H), 6.51 (s, 1H), 2.13 (s, 3H).

Synthesis of 7-(dimethylamino)-4-(o-tolyl)-2H-chromen-2-one, Example 1 [Step 2]: To a solution of [4-(o-tolyl)-2-oxo-chromen-7-yl]trifluoromethanesulfonate (2, 150 mg, 0.390 mmol) in 1,4 dioxane (5 mL) at ambient temperature was added N-methylmethanamine (2.0 mL, 3.90 mmol, 2M solution) and cesium carbonate (381 mg, 1.17 mmol). The reaction mixture was degassed with nitrogen, and Pd₂(dba)₃ (36 mg, 0.0390 mmol) added, followed by tBuXPhos (66 mg, 0.156 mmol). The reaction mixture was further degassed with nitrogen, then heated to 100° C. and stirred for 12 h. TLC analysis showed formation of new spots. This reaction mixture was purified by reverse phase prep-HPLC and dried by lyophilization to give 7-(dimethylamino)-4-(o-tolyl) chromen-2-one (Example 1, 10 mg). LCMS (ESI) Calcd. for C₁₈H₁₇NO₂: 279, found: [M+H]⁺=280. ¹H NMR (400 MHz, DMSO-d₆) δ 7.41-7.33 (m, 3H), 7.21 (d, 1H), 6.73 (d, 1H), 6.66-6.64 (m, 2H), 5.90 (s, 1H), 3.01 (s, 6H), 2.11 (s, 3H).

### Example 2: Synthesis of N-methyl-2-oxo-4-(o-tolyl)-2H-chromene-7-carboxamide

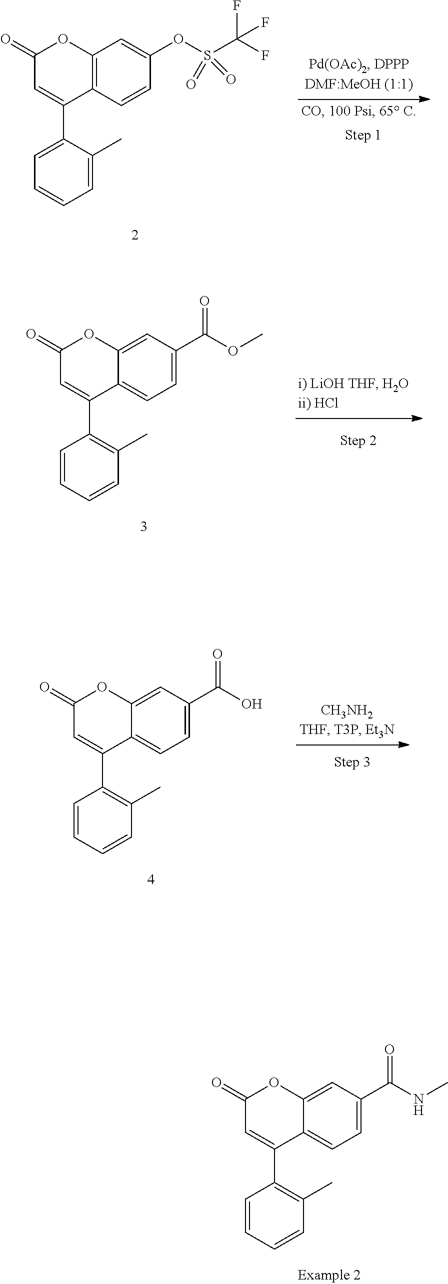 [SMILES: Cc1ccccc1-c1cc(=O)oc2cc(OS(=O)(=O)C(F)(F)F)ccc12 ; COC(=O)c1ccc2c(-c3ccccc3C)cc(=O)oc2c1 ; Cc1ccccc1-c1cc(=O)oc2cc(C(=O)O)ccc12 ; CNC(=O)c1ccc2c(-c3ccccc3C)cc(=O)oc2c1]

Synthesis of methyl 2-oxo-4-(o-tolyl)-2H-chromene-7-carboxylate, 3 [Step 1]: To a solution of [4-(o-tolyl)-2-oxo-chromen-7-yl]trifluoromethanesulfonate (2, 600 mg, 1.56 mmol) in DMF (6 mL) and methanol (6 mL) was added DPPP (64 mg, 0.156 mmol) followed by palladium acetate (35 mg, 0.156 mmol). The reaction mixture was pressurized with CO to 100 psi and heated to 65° C. The reaction was monitored by TLC analysis to completion. The reaction mixture was then cooled to ambient temperature and filtered through a celite bed. The bed was further washed with methanol. The combined filtrate solutions were concentrated under reduced pressure and purified by flash column chromatography on silica gel to afford methyl 4-(o-tolyl)-2-oxo-chromene-7-carboxylate (3, 350 mg). LCMS (ESI) Calcd. for C₁₈H₁₄O₄: 294, found: [M+H]⁺=295. ¹H NMR (400 MHz, DMSO-d₆) δ 7.93 (s, 1H), 7.83 (dd, 1H), 7.55-7.31 (m, 3H), 7.29 (d, 1H), 7.13 (d, 1H), 6.54 (s, 1H), 3.90 (s, 3H), 2.11 (s, 3H).

Synthesis of 2-oxo-4-(o-tolyl)-2H-chromene-7-carboxylic acid, 4 [Step 2]: To a solution of methyl 4-(o-tolyl)-2-oxo-chromene-7-carboxylate (3, 350 mg, 1.19 mmol) in THF (8 mL) was added 1N aqueous LiOH solution (6.0 mL) dropwise. The reaction mixture was stirred for 12 h. at ambient temperature. TLC analysis then showed formation of new polar spots. The reaction mixture was acidified with 2N aqueous HCl solution. The product was filtered, washed further with water, then pentane, and dried under reduced pressure to give 4-(o-tolyl)-2-oxo-chromene-7-carboxylic acid (4, 300 mg). LCMS (ESI) Calcd. for C₁₇H₁₂O₄: 280, found: [M+H]⁺=281. ¹H NMR (400 MHz, DMSO-d₆) δ 14.50-12.76 (m, 1H), 7.90 (s, 1H), 7.80 (d, 1H), 7.51-7.33 (m, 3H), 7.30 (d, 1H), 7.09 (d, 1H), 6.52 (s, 1H), 2.12 (s, 3H).

Synthesis of N-methyl-2-oxo-4-(o-tolyl)-2H-chromene-7-carboxamide, Example 2 [Step 3]: To a solution of 4-(o-tolyl)-2-oxo-chromene-7-carboxylic acid (4, 200 mg, 0.714 mmol) in THF (5 mL) at ambient temperature was added methylamine, 1M in THF (1 mL) followed by Et₃N (0.20 mL, 1.43 mmol). The reaction mixture was then cooled to 0° C. and T₃P (0.25 mL, 0.856 mmol) was added. The mixture was warmed to ambient temperature and stirred for 3 h. The reaction was monitored by LCMS analysis. After completion, the reaction mixture was diluted with CH₂Cl₂ and washed with 10% aqueous NaHCO₃ solution, then brine, dried over anhydrous sodium sulfate, filtered, then concentrated under reduced pressure. The product was purified by reverse phase prep-HPLC and dried by lyophilization to afford N-methyl-2-oxo-4-(o-tolyl)-2H-chromene-7-carboxamide (Example 2, 80 mg). LCMS (ESI) Calcd. for C₁₈H₁₅NO₃: 293, found: [M+H]⁺=294. ¹H NMR (400 MHz, DMSO-d₆) δ 8.34 (s, 1H), 7.87 (s, 1H), 7.69 (d, 1H), 7.50-7.33 (m, 3H), 7.27 (d, 1H), 7.06 (d, 1H), 6.40 (s, 1H), 2.84 (d, 3H), 2.14 (s, 3H).

### Examples 3-5: Synthesis of 4 4-(2-chloro-4-fluorophenyl)-7-methyl-2H-chromen-2-one, 4-(2-chloro-4-fluorophenyl)-7-((methylamino)methyl)-2H-chromen-2-one, and N-((4-(2-chloro-4-fluorophenyl)-2-oxo-2H-chromen-7-yl)methyl)-N-methylpiperidine-1-carboxamide

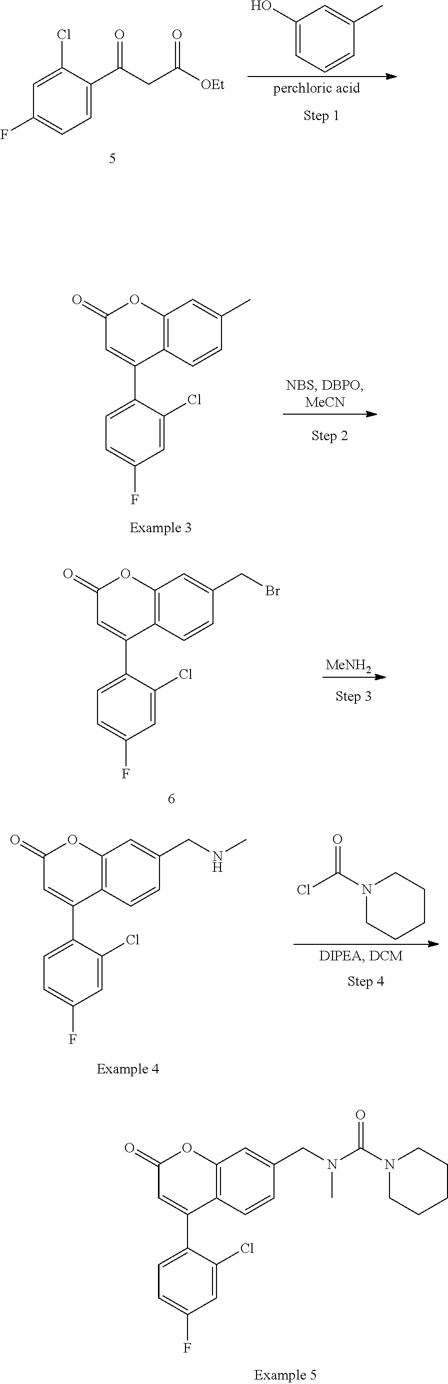

Synthesis of 4-(2-chloro-4-fluorophenyl)-7-methyl-2H-chromen-2-one, Example 3 [Step 1]: A mixture of ethyl 3-(2-chloro-4-fluoro-phenyl)-3-oxo-propanoate (5, 100 mg, 0.409 mmol) and m-cresol (44 mg, 0.409 mmol) in perchloric acid (0.025 mL, 0.409 mmol) was stirred at ambient temperature for 48 h. TLC analysis showed complete conversion of starting material with formation of a new polar spot. The reaction mixture was then poured into ice water and extracted with CH₂Cl₂. The organic layer was separated, washed with water, then brine, dried over anhydrous sodium sulfate, filtered, then concentrated under at reduced pressure. The product was purified by reverse phase prep-HPLC and lyophilized to give 4-(2-chloro-4-fluorophenyl)-7-methyl-2H-chromen-2-one (Example 3, 37 mg). LCMS (ESI) Calcd. for C₁₆H₁₀ClFO₂: 288, found: [M+H]⁺=289. ¹H NMR (400 MHz, DMSO-d₆) δ 7.71 (dd, 1H), 7.58 (dd, 1H), 7.49-7.39 (m, 1H), 7.34 (s, 1H), 7.14 (d, 1H), 6.93 (d, 1H), 6.43 (s, 1H), 2.41 (s, 3H).

Synthesis of 7-(bromomethyl)-4-(2-chloro-4-fluorophenyl)-2H-chromen-2-one, 6 [Step 2]: To a stirred solution of 4-(2-chloro-4-fluorophenyl)-7-methyl-2H-chromen-2-one (Example 3, 200 mg, 0.693 mmol) in MeCN (3 mL) was added NBS (148 mg, 0.831 mmol) and DBPO (17 mg, 0.0693 mmol). The resulting mixture was heated to 80° C. for 4 h. and then stirred at ambient temperature overnight. TLC analysis then showed full conversion of starting material with formation of new polar spot. The reaction mixture was concentrated under reduced pressure and the residue was extracted with EtOAc and water. The organic phase was separated, washed with brine, dried over anhydrous sodium sulfate, filtered, then concentrated under reduced pressure. The product was purified by flash column chromatography on silica gel to give 7-(bromomethyl)-4-(2-chloro-4-fluorophenyl)-2H-chromen-2-one (6, 110 mg). LCMS (ESI) Calcd. for C₁₆H₉BrClFO₂: 368, found: [M+H]⁺=369. ¹H NMR (400 MHz, DMSO-d₆): δ 7.71-7.73 (m, 1H), 7.57-7.64 (m, 2H), 7.44-7.47 (m, 1H), 7.37-7.39 (m, 1H), 7.04-7.06 (m, 1H), 6.54 (s, 1H), 7.79 (s, 2H).

Synthesis of 4-(2-chloro-4-fluorophenyl)-7-((methylamino)methyl)-2H-chromen-2-one (formate salt), Example 4 [Step 3]: A sealed tube containing 7-(bromomethyl)-4-(2-chloro-4-fluoro-phenyl) chromen-2-one (6, 100 mg, 0.272 mmol) and MeNH₂ in THF (2.0 mL) was heated to 60° C. for 16 h. TLC analysis then showed full conversion of starting material with formation of a new polar spot. The mixture was concentrated under reduced pressure and purified by flash column chromatography on silica gel followed by reverse phase prep-HPLC and lyophilization to give 4-(2-chloro-4-fluorophenyl)-7-((methylamino)methyl)-2H-chromen-2-one (Example 4, 25 mg) as the formate salt. LCMS (ESI) Calcd. for C₁₇H₁₃ClFNO₂: 317, found: [M+H]⁺=318. ¹H NMR (400 MHz, DMSO-d₆) δ 8.25 (s, 1H), 7.72 (dd, 1H), 7.59 (dd, 1H), 7.51-7.41 (m, 2H), 7.28 (d, 1H), 7.00 (d, 1H), 6.48 (s, 1H), 3.80 (s, 2H), 2.30 (s, 3H).

Synthesis of N-((4-(2-chloro-4-fluorophenyl)-2-oxo-2H-chromen-7-yl)methyl)-N-methylpiperidine-1-carboxamide, Example 5 [Step 4]: To a stirred solution of 4-(2-chloro-4-fluorophenyl)-7-((methylamino)methyl)-2H-chromen-2-one (Example 4, 60 mg, 0.189 mmol) in CH₂Cl₂ (3 mL) at 0° C. was added DIPEA (0.051 mL, 0.378 mmol) and piperidine-1-carbonyl chloride (28 mg, 0.189 mmol). The resulting mixture was warmed to ambient temperature and stirred for 16 h. TLC analysis then showed full conversion of starting material with formation of a new polar spot. The reaction mixture was diluted with CH₂Cl₂, washed with water, then brine, dried over anhydrous sodium sulfate, filtered, then concentrated under reduced pressure. The product was purified by reverse phase prep-HPLC and lyophilized to give N-((4-(2-chloro-4-fluorophenyl)-2-oxo-2H-chromen-7-yl)methyl)-N-methylpiperidine-1-carboxamide (Example 5, 17 mg). LCMS (ESI) Calcd. for C₂₃H₂₂ClFN₂O₃: 428, found: [M+H]⁺=429. ¹H NMR (400 MHz, DMSO-d₆) δ ¹H NMR (400 MHz, DMSO-d₆) δ 7.72 (dd, 1H), 7.58 (dd, 1H), 7.49-7.40 (m, 1H), 7.34 (s, 1H), 7.19 (dd, 1H), 7.02 (d, 1H), 6.49 (s, 1H), 4.40 (s, 2H), 3.12 (t, 4H), 2.72 (s, 3H), 1.50 (t, 6H).

### Examples 6-8: Synthesis of 1-((4-(2-chloro-4-fluorophenyl)-2-oxo-2H-chromen-7-yl)methyl)-1,3,3-trimethylurea, (S)-ethyl 1-(((4-(2-chloro-4-fluorophenyl)-2-oxo-2H-chromen-7-yl)methyl)(methyl)carbamoyl)piperidine-3-carboxylate, and (S)-1-(((4-(2-chloro-4-fluorophenyl)-2-oxo-2H-chromen-7-yl)methyl)(methyl)carbamoyl)piperidine-3-carboxylic acid

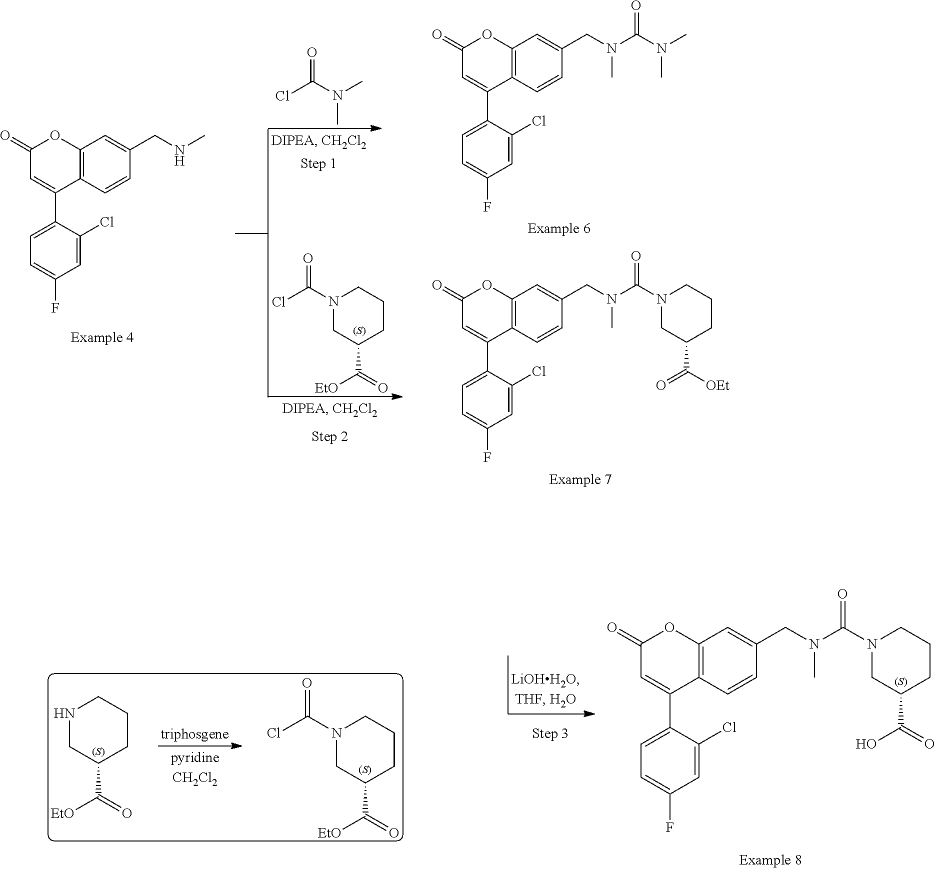

Synthesis of 1-((4-(2-chloro-4-fluorophenyl)-2-oxo-2H-chromen-7-yl)methyl)-1,3,3-trimethylurea, Example 6 [Step 1]: To a stirred solution of 4-(2-chloro-4-fluorophenyl)-7-((methylamino)methyl)-2H-chromen-2-one (Example 4, 60 mg, 0.2 mmol) in CH₂Cl₂ (3 mL) at 0° C. were added DIPEA (0.05 mL, 0.4 mmol) and N,N-dimethylcarbamoyl chloride (0.02 mL, 0.2 mmol). The reaction mixture was warmed to ambient temperature and stirred for 16 h. TLC analysis at that time showed full conversion of starting material with formation of a new polar spot. The reaction mixture was diluted with CH₂Cl₂, washed with water, then brine, dried over anhydrous sodium sulfate, filtered, and concentrated under reduced pressure. The product was purified by reverse phase prep-HPLC and lyophilization to afford 1-((4-(2-chloro-4-fluorophenyl)-2-oxo-2H-chromen-7-yl)methyl)-1,3,3-trimethylurea (Example 6, 31 mg). LCMS (ESI) Calcd. for C₂₀H₁₈ClFN₂O₃: 388, found: [M+H]⁺=389. ¹H NMR (400 MHz, DMSO-d₆) δ 7.72 (dd, 1H), 7.60-7.56 (m, 1H), 7.47-7.42 (m, 1H), 7.36 (s, 1H), 7.20 (d, 1H), 7.02 (d, 1H), 6.49 (d, 1H), 4.39 (s, 2H), 2.75 (s, 6H), 2.71 (s, 3H).

Synthesis of ethyl (S)-1-(((4-(2-chloro-4-fluorophenyl)-2-oxo-2H-chromen-7-yl)methyl)(methyl)carbamoyl)piperidine-3-carboxylate, Example 7 [Step 2]: To a stirred solution of 4-(2-chloro-4-fluorophenyl)-7-((methylamino)methyl)-2H-chromen-2-one (Example 4, 100 mg, 0.3 mmol) in CH₂Cl₂ (5 mL) at 0° C. were added DIPEA (0.13 mL, 1.0 mmol) and ethyl (S)-1-chlorocarbonylpiperidine-3-carboxylate (76 mg, 0.3 mmol). The reaction mixture was warmed to ambient temperature and stirred for 16 h. TLC analysis at that time showed full conversion of starting material with formation of a new polar spot. The reaction mixture was diluted with CH₂Cl₂, washed with water, then brine, dried over anhydrous sodium sulfate, filtered, and concentrated under reduced pressure. The product was purified by reverse phase prep-HPLC and lyophilization to yield ethyl (S)-1-(((4-(2-chloro-4-fluorophenyl)-2-oxo-2H-chromen-7-yl)methyl)(methyl)carbamoyl)piperidine-3-carboxylate (Example 7, 31 mg). LCMS (ESI): Calcd. for C₂₆H₂₆ClFN₂O₅: 500, found: [M+H]⁺=501. ¹H NMR (400 MHz, DMSO-d₆) δ 7.72 (dd, 1H), 7.60-7.56 (m, 1H), 7.47-7.43 (m, 1H), 7.35 (s, 1H), 7.19 (d, 1H), 7.02 (d, 1H), 6.49 (d, 1H), 4.42 (s, 2H), 4.05 (q, 2H), 3.60 (d, 1H), 3.40 (d, 1H), 2.97-2.86 (m, 2H), 2.73 (s, 3H), 2.55-2.54 (m, 1H), 1.93-1.89 (m, 1H), 1.62-1.49 (m, 3H), 1.14 (t, 3H).

Ethyl(S)-1-(chlorocarbonyl)piperidine-3-carboxylate was prepared in the following manner: To a stirred solution of triphosgene (1.0 eq, 560 mg, 1.9 mmol) in CH₂Cl₂ (5 mL) at 0° C. was added dropwise pyridine (2.00 eq, 0.31 mL, 3.8 mmol). The reaction mixture was stirred for 30 min., then ethyl (3S)-piperidine-3-carboxylate (1.0 eq, 300 mg, 1.9 mmol) was added. The reaction mixture was stirred for 1 h. more, at which time TLC analysis showed complete conversion of starting material had occurred. The reaction mixture was diluted with CH₂Cl₂, washed with 1N aqueous HCl solution, water, then brine, dried over anhydrous sodium sulfate, filtered, and concentrated under reduced pressure to yield ethyl (S)-1-(chlorocarbonyl)piperidine-3-carboxylate (350 mg).

Synthesis of (S)-1-(((4-(2-chloro-4-fluorophenyl)-2-oxo-2H-chromen-7-yl)methyl)(methyl)carbamoyl)piperidine-3-carboxylic acid, Example 8 [Step 3]: To a stirred solution ethyl (S)-1-(((4-(2-chloro-4-fluorophenyl)-2-oxo-2H-chromen-7-yl)methyl)(methyl)carbamoyl)piperidine-3-carboxylate (Example 7, 50 mg, 0.1 mmol) in THF (3 mL):water (1 mL) mixture was added LiOH·H₂O (5.0 mg, 0.1 mmol). The reaction mixture was stirred for 16 h., at which time TLC analysis showed full conversion of starting material with formation of new polar spot. The mixture was concentrated under reduced pressure and the residue was acidified with 1N aqueous HCl solution to pH=3. The aqueous layer was separated and extracted with EtOAc. The combined organic extracts were dried over sodium sulfate, filtered and concentrated under reduced pressure. The product was purified by reverse phase prep-HPLC and lyophilization to yield(S)-1-(((4-(2-chloro-4-fluorophenyl)-2-oxo-2H-chromen-7-yl)methyl)(methyl)carbamoyl)piperidine-3-carboxylic acid (Example 8, 14 mg). LCMS (ESI): Calcd. for C₂₄H₂₂ClFN₂O₅: 472, found: [M+H]⁺=473. ¹H NMR (400 MHz, DMSO-d₆) δ 7.72 (dd, 1H), 7.60-7.57 (m, 1H), 7.47-7.42 (m, 1H), 7.36 (br s, 1H), 7.19 (d, 1H), 7.02 (d, 1H), 4.42 (q, 2H), 3.61 (d, 1H), 3.42-3.88 (m, 2H), 2.91-2.82 (m, 2H), 2.73 (s, 3H), 2.43 (br s, 1H), 1.90 (br s, 1H), 1.62 (br s, 1H), 1.55-1.45 (m, 2H).

### Examples 9 and 10: Synthesis of 7-(hydroxymethyl)-4-(o-tolyl)-2H-chromen-2-one and 7-(methoxymethyl)-4-(o-tolyl)-2H-chromen-2-one

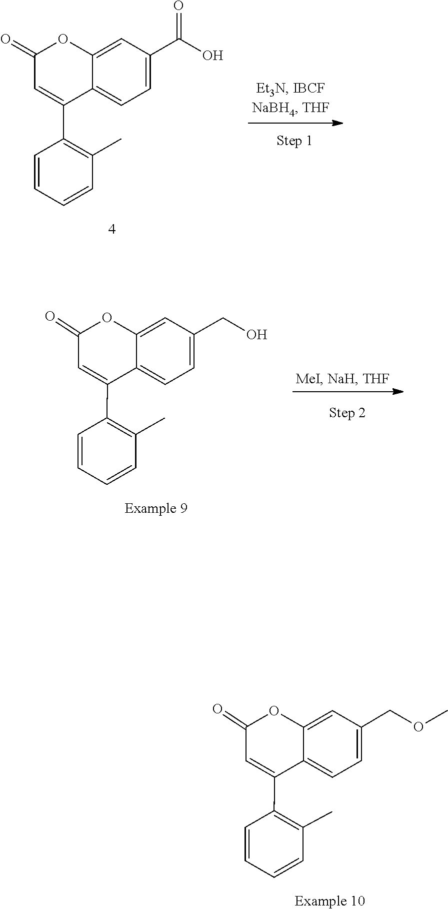 [SMILES: Cc1ccccc1-c1cc(=O)oc2cc(C(=O)O)ccc12 ; Cc1ccccc1-c1cc(=O)oc2cc(CO)ccc12 ; COCc1ccc2c(-c3ccccc3C)cc(=O)oc2c1]

Synthesis of 7-(hydroxymethyl)-4-(o-tolyl)-2H-chromen-2-one, Example 9 [Step 4]: To a solution of 4-(o-tolyl)-2-oxo-chromene-7-carboxylic acid (4, 500 mg, 1.78 mmol) in THF (15 mL) at ambient temperature was added triethyl amine (0.99 mL, 7.14 mmol). The mixture was cooled to 0° C. and isobutyl chloroformate (726 mL, 5.6 mmol) added dropwise. The reaction mixture was stirred at 0° C. for 1 h., then a solution of NaBH₄ (339 mg, 8.92 mmol) in water (5 mL) was added. The resulting mixture was stirred overnight at ambient temperature. It was then poured into a saturated aqueous ammonium chloride solution and extracted with EtOAc. The combined organic layers were washed with water, then brine, dried over anhydrous magnesium sulfate, filtered, then concentrated under reduced pressure. The product was purified by flash column chromatography on silica gel to give 7-(hydroxymethyl)-4-(o-tolyl)-2H-chromen-2-one (Example 9, 350 mg). LCMS (ESI) Calcd. for C₁₇H₁₄O₃: 266, found: [M+H]⁺=267. ¹H NMR (400 MHz, DMSO-d₆) δ 7.49-7.31 (m, 4H), 7.30-7.20 (m, 2H), 6.93 (d, 1H), 6.33 (s, 1H), 5.46 (t, 1H), 4.60 (d, 2H), 2.11 (s, 3H).

Synthesis of 7-(methoxymethyl)-4-(o-tolyl)-2H-chromen-2-one, Example 10 [Step 5]: To a solution of 7-(hydroxymethyl)-4-(o-tolyl)-2H-chromen-2-one (Example 9, 100 mg, 0.376 mmol) in THF (2 mL) at 0° C. was added sodium hydride (14 mg, 0.563 mmol). Methyl iodide (0.47 mL, 7.51 mmol) was added, and the mixture stirred for 12 h. The reaction was then quenched with saturated aqueous NH₄Cl solution and extracted with EtOAc. The combined organic layers were dried over anhydrous sodium sulfate, filtered, then concentrated under reduced pressure. Purification by reverse phase prep-HPLC gave 7-(methoxymethyl)-4-(o-tolyl)-2H-chromen-2-one (Example 10, 10 mg). LCMS (ESI) Calcd. for C₁₈H₁₆O₃: 280, found: [M+H]⁺=281. ¹H NMR (400 MHz, DMSO-d₆) δ 7.50-7.32 (m, 4H), 7.30-7.20 (m, 2H), 6.97 (d, 1H), 6.37 (s, 1H), 4.52 (s, 2H), 3.33 (s, 3H), 2.11 (s, 3H).

### Example 11: Synthesis of 7-amino-4-(2-chloro-4-fluoro-phenyl) chromen-2-one

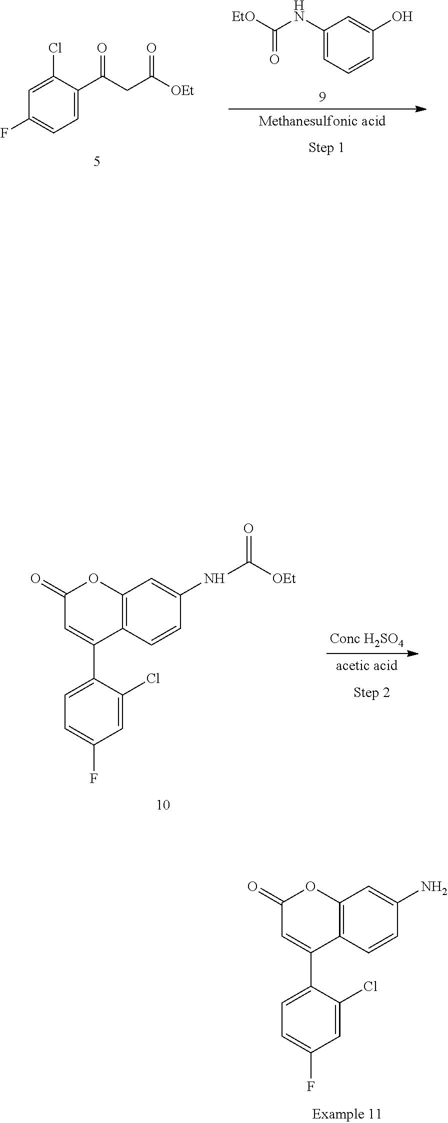 [SMILES: CCOC(=O)CC(=O)c1ccc(F)cc1Cl ; CCOC(=O)Nc1cccc(O)c1 ; CCOC(=O)Nc1ccc2c(-c3ccc(F)cc3Cl)cc(=O)oc2c1 ; Nc1ccc2c(-c3ccc(F)cc3Cl)cc(=O)oc2c1]

Synthesis of ethyl N-(4-(2-chloro-4-fluorophenyl)-2-oxo-2H-chromen-7-yl)carbamate, 10 [Step 1]: To a stirred solution of ethyl 3-(2-chloro-4-fluoro-phenyl)-3-oxo-propanoate (5, 200 mg, 0.817 mmol) in methanesulfonic acid (0.50 mL, 7.70 mmol) at ambient temperature was added ethyl (3-hydroxyphenyl)carbamate (9, 148 mg, 0.817 mmol). The resulting mixture was stirred for 16 h. then quenched with cold water and extracted with CH₂Cl₂. The organic layer was separated, washed with water, then brine, dried with anhydrous sodium sulfate filtered, then concentrated under reduced pressure. The product was purified by flash column chromatography on silica gel to give ethyl (4-(2-chloro-4-fluorophenyl)-2-oxo-2H-chromen-7-yl)carbamate (10, 180 mg). LCMS (ESI) Calcd. for C₁₈H₁₃ClFNO₄: 361, found: [M+H]⁺=362. ¹H NMR (400 MHz, Chloroform-d) δ 7.54 (br s, 1H), 7.29-6.54 (m, 6H), 6.22 (s, 1H), 4.27-4.15 (m, 2H), 1.33-1.27 (m, 3H).

Synthesis of 7-amino-4-(2-chloro-4-fluorophenyl)-2H-chromen-2-one, Example 11 [Step 2]: A suspension of ethyl N-[4-(2-chloro-4-fluoro-phenyl)-2-oxo-chromen-7-yl]carbamate (10, 170 mg, 0.470 mmol) in conc. H₂SO₄ (1.0 mL, 0.470 mmol) and glacial acetic acid (0.57 mL, 10.0 mmol) was heated to 120° C. for 4 h. The reaction mixture was then cooled to ambient temperature, poured into ice water, neutralized with 50% aqueous NaOH solution (pH: 7), then extracted with 6% MeOH in CH₂Cl₂. Combined organic extracts were dried over anhydrous sodium sulfate, filtered, then concentrated under reduced pressure. The product was purified by flash column chromatography to give 7-amino-4-(2-chloro-4-fluorophenyl)-2H-chromen-2-one (Example 11, 70 mg). LCMS (ESI) Calcd. for C₁₅H₉ClFNO₂: 289, found: [M+H]⁺=292. ¹H NMR (400 MHz, Chloroform-d) δ 7.31-7.24 (m, 1H), 7.16-7.06 (m, 1H), 6.80 (d, 1H), 6.60 (d, 1H), 6.46 (dd, 1H), 6.04 (s, 1H), 4.20 (s, 2H).

### Example 12: Synthesis of 4-(4-fluorophenyl)-5-hydroxy-7-methyl-2H-chromen-2-one

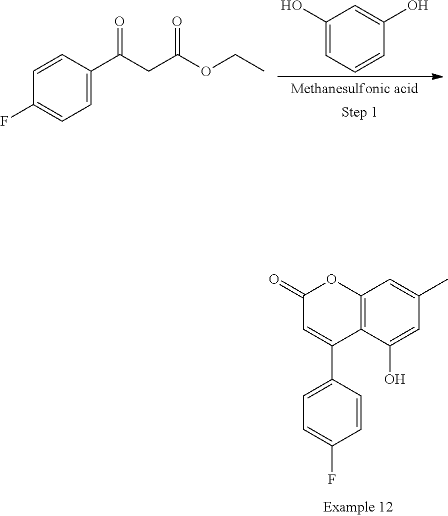 [SMILES: CCOC(=O)CC(=O)c1ccc(F)cc1 ; Oc1cccc(O)c1 ; Cc1cc(O)c2c(-c3ccc(F)cc3)cc(=O)oc2c1]

Synthesis of 4-(4-fluorophenyl)-5-hydroxy-7-methyl-2H-chromen-2-one, Example 12 [Step 1]: To a mixture of ethyl 3-(4-fluorophenyl)-3-oxo-propanoate (1.00 g, 4.76 mmol) and 5-methylbenzene-1,3-diol (650 mg, 5.23 mmol) was added methanesulphonic acid (2.0 mL) dropwise with constant stirring. The reaction mixture was heated to 50° C. for 6 h., then cooled to 0° C. and quenched with water. The resulting product was filtered, washed with water, then n-pentane, and dried under reduced pressure to afford 4-(4-fluorophenyl)-5-hydroxy-7-methyl-2H-chromen-2-one (Example 12, 800 mg). ¹H NMR (400 MHz, DMSO-d₆) δ 10.19 (s, 1H), 7.40 (dd, 2H), 7.20 (t, 2H), 6.72 (s, 1H), 6.48 (s, 1H), 5.99 (s, 1H), 2.29 (s, 3H). LCMS (ESI) Calcd. for C₁₆H₁FO₃: 270, found [M+H]⁺=271.

### Examples 13-15: Synthesis of 4-(2-chloro-4-fluorophenyl)-7-(hydroxymethyl)-2H-chromen-2-one, ethyl (E)-3-(4-(2-chloro-4-fluorophenyl)-2-oxo-2H-chromen-7-yl)-2-methylacrylate, and methyl 3-(4-(2-chloro-4-fluorophenyl)-2-oxo-2H-chromen-7-yl)-2-methylpropanoate

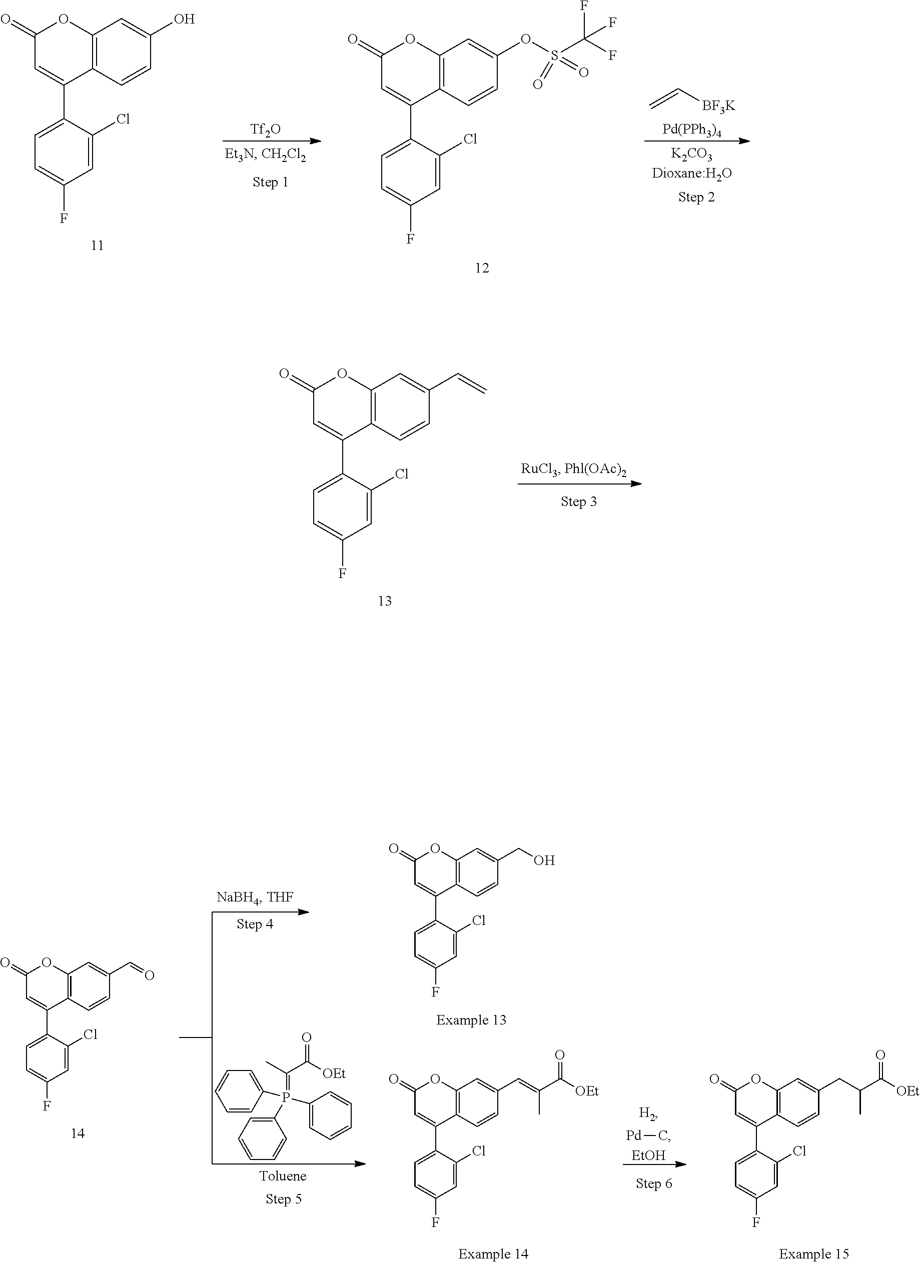

Synthesis of 4-(2-chloro-4-fluorophenyl)-2-oxo-2H-chromen-7-yl trifluoromethanesulfonate, 12 [Step 1]: To a stirred solution of 4-(2-chloro-4-fluorophenyl)-7-hydroxy-2H-chromen-2-one (11, 1.00 eq, 300 mg, 1 mmol) in CH₂Cl₂ (3 mL) at 0° C. was added TEA (0.2 mL) followed by trifluoromethanesulfonic anhydride (0.3 mL) and the reaction mixture was stirred at ambient temperature for 16 h. At that time the mixture was diluted with EtOAc and washed with water, then brine. The organic phase was separated, dried over anhydrous sodium sulfate, filtered, and concentrated under reduced pressure. The product was purified by flash column chromatography on silica gel to afford 4-(2-chloro-4-fluorophenyl)-2-oxo-2H-chromen-7-yl trifluoromethanesulfonate (12, 360 mg). LCMS (ESI): Calcd. for C₁₆H₇ClF₄O₅S: 422, found: [M+H]⁺=423. ¹H NMR (400 MHz, DMSO-d₆) δ 7.87 (d, 1H), 7.74 (dd, 1H), 7.60 (dd, 1H), 7.49-7.43 (m, 2H), 7.24 (d, 1H), 6.67 (s, 1H).

Synthesis of 4-(2-chloro-4-fluorophenyl)-7-vinyl-2H-chromen-2-one, 13 [Step 2]: To a stirred solution 4-(2-chloro-4-fluorophenyl)-2-oxo-2H-chromen-7-yl trifluoromethanesulfonate (12, 1.00 eq, 350 mg, 0.9 mmol) in a mixture of dioxane (8 mL) and water (2 mL) was added potassium vinyltrifluoroborate (1.30 eq, 160 mg, 1.2 mmol), and K₂CO₃ (3.00 eq, 360 mg, 2.6 mmol). The reaction mixture was degassed with argon for 10 min. and Pd(PPh₃)₄ (0.100 eq, 100 mg, 0.1 mmol) was added. The reaction mixture was then heated to 100° C. for 12 h. The reaction was monitored by TLC analysis until completion of reaction. At that time the reaction mixture was filtered through a celite bed. The filtrate was diluted with EtOAc, washed with water, then brine, dried over anhydrous sodium sulfate, and concentrated under reduced pressure. The product was purified by flash column chromatography on silica gel to afford 4-(2-chloro-4-fluorophenyl)-7-vinyl-2H-chromen-2-one (13, 190 mg). LCMS (ESI) Calcd. for C₁₇H₁₀ClFO₂: 300, found: [M+H]⁺=301. ¹H NMR (400 MHz, DMSO-d₆) δ 7.72 (dd, 1H), 7.63-7.57 (m, 2H), 7.48-7.42 (m, 2H), 7.01 (d, 1H), 6.87-6.80 (m, 1H), 6.49 (s, 1H), 6.07 (d, 1H), 5.48 (d, 1H).

Synthesis of 4-(2-chloro-4-fluorophenyl)-2-oxo-2H-chromene-7-carbaldehyde, 14 [Step 3]: To a stirred solution of 4-(2-chloro-4-fluorophenyl)-7-vinyl-2H-chromen-2-one (13, 1.00 eq, 190 mg, 0.6 mmol) in CH₂C₁₂: H₂O (4 mL: 1 mL) mixture was added RuCl₃ (0.100 eq, 15 mg, 0.07 mmol) and PhI(OAc)₂ (3.00 eq, 610 mg, 1.9 mmol). The reaction mixture was stirred at ambient temperature for 1 h. After TLC and LCMS analyses showed reaction completion, the reaction mixture was filtered through a celite bed. The filtrate was diluted with CH₂Cl₂, washed with water, then brine, dried over anhydrous sodium sulfate, and concentrated under reduced pressure. The product was purified by flash column chromatography on silica gel to afford 4-(2-chloro-4-fluorophenyl)-2-oxo-2H-chromene-7-carbaldehyde (14, 80 mg). LCMS (ESI) Calcd. for C₁₆H₈ClFO₃: 302, found: [M+H]⁺=303. ¹H NMR (400 MHz, DMSO-d₆) δ 10.10 (s, 1H), 8.00 (s, 1H), 7.80 (d, 1H), 7.75 (dd, 1H), 7.62 (dd, 1H), 7.48 (dt, 1H), 7.27 (d, 1H), 6.72 (s, 1H).

Synthesis of 4-(2-chloro-4-fluorophenyl)-7-(hydroxymethyl)-2H-chromen-2-one, Example 13 [Step: 4]: A stirred solution of 4-(2-chloro-4-fluorophenyl)-2-oxo-2H-chromene-7-carbaldehyde (14, 1.00 eq, 80 mg, 0.3 mmol) in THF (2 mL) at 0° C. was added NaBH₄ (20 mg, 0.5 mmol). The reaction mixture was allowed to warm to ambient temperature and stirred for 2 h. TLC analysis at that time showed complete consumption of starting material and formation of new polar spot. The reaction mixture was diluted with EtOAc, washed with water, then brine, dried over anhydrous sodium sulfate, and concentrated under reduced pressure. The product was purified by flash column chromatography on silica gel to afford 4-(2-chloro-4-fluorophenyl)-7-(hydroxymethyl)-2H-chromen-2-one (Example 13, 70 mg). LCMS (ESI): Calcd. for C₁₆H₁₀ClFO₃: 304, found: [M+H]⁺=305 ¹H NMR (400 MHz, DMSO-d₆) δ 7.71 (dd, 1H), 7.59 (dd, 1H), 7.47-7.41 (m, 2H), 7.25 (d, 1H), 7.01 (d, 1H), 6.47 (s, 1H), 5.45 (t, 1H), 4.60 (d, 2H).

Synthesis of ethyl (E)-3-(4-(2-chloro-4-fluorophenyl)-2-oxo-2H-chromen-7-yl)-2-methylacrylate, Example 14 [Step 5]: To a stirred solution of ethyl 2-(trimethyl-λ⁵-phosphanylidene)propanoate (1.2 eq, 60 mg, 0.3 mmol) in toluene (5 mL) at ambient temperature under an argon atmosphere was added a solution of 4-(2-chloro-4-fluorophenyl)-2-oxo-2H-chromene-7-carbaldehyde (14, 1.00 eq, 80 mg, 0.3 mmol) in toluene. The reaction mixture was stirred at ambient temperature for 16 h., at which time TLC analysis showed complete consumption of starting material and formation of new polar spot. The reaction mixture was concentrated under reduced pressure and the resulting mixture was diluted with EtOAc, washed with water, then brine, dried over anhydrous sodium sulfate, and concentrated under reduced pressure. This product was purified by flash column chromatography on silica gel to afford ethyl (E)-3-(4-(2-chloro-4-fluorophenyl)-2-oxo-2H-chromen-7-yl)-2-methylacrylate (Example 14, 70 mg). LCMS (ESI): Calcd. for C₂₁H₁₆ClFO₄: 386, found: [M+H]⁺=387. ¹H NMR (400 MHz, DMSO-d₆) δ 7.73 (dd, 1H), 7.65-7.59 (m, 3H), 7.48-7.44 (m, 1H), 7.41 (dd, 1H), 7.09 (d, 1H), 6.57 (s, 1H), 4.22 (q, 2H), 2.09 (s, 3H), 1.28 (t, 3H).

Synthesis of methyl 3-(4-(2-chloro-4-fluorophenyl)-2-oxo-2H-chromen-7-yl)-2-methylpropanoate, Example 15 [Step 6]: A solution of ethyl (E)-3-(4-(2-chloro-4-fluorophenyl)-2-oxo-2H-chromen-7-yl)-2-methylacrylate (Example 14, 1.00 eq, 100 mg, 0.3 mmol) in EtOH (3 mL) was degassed with argon for 10 min. 10% Pd—C(10 mg) was then added under argon atmosphere and the reaction mixture allowed to stir under a hydrogen atmosphere for 1 h. at ambient temperature, at which time TLC analysis showed complete consumption of starting material and formation of new polar spot. The reaction mixture was filtered through celite bed. The filtrate was concentrated under reduced pressure and the product was purified by reverse phase prep-HPLC to afford methyl 3-(4-(2-chloro-4-fluorophenyl)-2-oxo-2H-chromen-7-yl)-2-methylpropanoate (Example 15, 50 mg). LCMS (ESI) Calcd. for C₂₁H₁₈ClFO₄: 388, found: [M+H]⁺=389. ¹H NMR (400 MHz, DMSO-d₆) δ 7.71 (dd, 1H), 7.58 (dd, 1H), 7.44 (dt, 1H), 7.35 (br s, 1H), 7.16-7.13 (m, 1H), 6.96 (d, 1H), 6.46 (s, 1H), 4.01 (q, 2H), 2.98-2.94 (m, 1H), 2.84-2.80 (m, 2H), 1.14-1.05 (m, 6H).

### Example 16: Synthesis of 4-(2-chloro-4-fluorophenyl)-7-(methoxymethyl)-2H-chromen-2-one

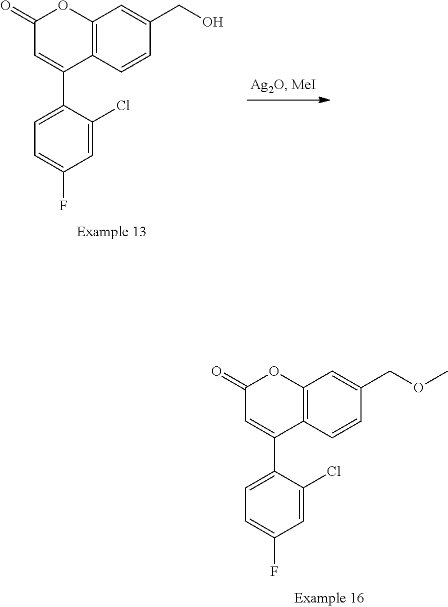 [SMILES: O=c1cc(-c2ccc(F)cc2Cl)c2ccc(CO)cc2o1 ; COCc1ccc2c(-c3ccc(F)cc3Cl)cc(=O)oc2c1]

Synthesis of 4-(2-chloro-4-fluorophenyl)-7-(methoxymethyl)-2H-chromen-2-one, Example 16: To a stirred solution of 4-(2-chloro-4-fluorophenyl)-7-(hydroxymethyl)-2H-chromen-2-one (Example 13, 30 mg, 0.1 mmol) in methyl iodide (0.2 mL, 3.0 mmol) at ambient temperature was added Ag₂O (46 mg, 0.2 mmol). The resulting mixture was stirred for 16 h., then filtered through a celite bed. The filtrate was diluted with EtOAc and washed with saturated aqueous NH₄Cl solution, then brine, dried over anhydrous sodium sulfate, filtered, and concentrated under reduced pressure. The product was purified by flash column chromatography on silica gel, then lyophilized to give 4-(2-chloro-4-fluorophenyl)-7-(methoxymethyl)-2H-chromen-2-one (Example 16, 14 mg). LCMS (ESI): Calcd. for C₁₇H₁₂ClFO₃: 318, found: [M+H]⁺=319. ¹H NMR (400 MHz, DMSO-d₆) δ 7.72 (dd, 1H), 7.59 (dd, 1H), 7.48-7.42 (m, 2H), 7.25 (d, 1H), 7.03 (d, 1H), 6.50 (s, 1H), 4.53 (s, 2H), 3.33 (s, 3H).

### Examples 17-21: Synthesis of 3-(4-(2-chloro-4-fluorophenyl)-2-oxo-2H-chromen-7-yl)-2-methylpropanoic acid, (S)-4-(2-chloro-4-fluorophenyl)-7-(2-methyl-3-oxo-3-(piperidin-1-yl)propyl)-2H-chromen-2-one, (R)-4-(2-chloro-4-fluorophenyl)-7-(2-methyl-3-oxo-3-(piperidin-1-yl)propyl)-2H-chromen-2-one, (S)-3-(4-(2-chloro-4-fluorophenyl)-2-oxo-2H-chromen-7-yl)-N,N,2-trimethylpropanamide, and (R)-3-(4-(2-chloro-4-fluorophenyl)-2-oxo-2H-chromen-7-yl)-N,N,2-trimethylpropanamide

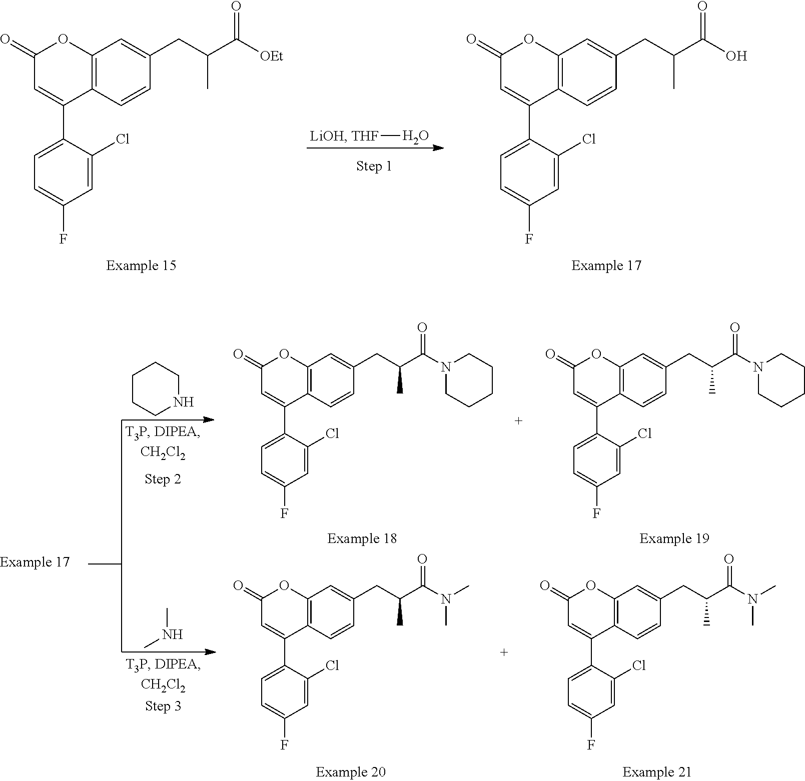

Synthesis of 3-(4-(2-chloro-4-fluorophenyl)-2-oxo-2H-chromen-7-yl)-2-methylpropanoic acid, Example 17 [Step 1]: To a stirred solution of ethyl 3-(4-(2-chloro-4-fluorophenyl)-2-oxo-2H-chromen-7-yl)-2-methylpropanoate (Example 15, 170 mg, 0.4 mmol) in THF (4 mL) and H₂O (1 mL) mixture at ambient temperature was added LiOH. H₂O (55 mg, 1.3 mmol). The resulting mixture was stirred for 16 h., at which time TLC and LCMS analyses showed complete conversion of starting material had occurred. The mixture was concentrated under reduced pressure, diluted with water, and extracted with EtOAc. The aqueous layer was separated, acidified with 1N aqueous HCl solution, then extracted with EtOAc. The organic extract was separated, washed with water, then brine, dried over anhydrous sodium sulfate, filtered, and concentrated under reduced pressure to afford 3-(4-(2-chloro-4-fluorophenyl)-2-oxo-2H-chromen-7-yl)-2-methylpropanoic acid (Example 17, 130 mg). LCMS (ESI): Calcd. for C₁₉H₁₄ClFO₄: 360, found: [M+H]⁺=361. ¹H NMR (400 MHz, DMSO-d₆) δ 12.23 (s, 1H), 7.71 (dd, 1H), 7.62-7.56 (m, 1H), 7.46-7.42 (m, 1H), 7.35 (br s, 1H), 7.16 (d, 1H), 6.95 (d, 1H), 6.46 (s, 1H), 3.17 (d, 3H), 3.00-2.98 (m, 1H), 2.75-2.72 (m, 2H).

Synthesis of (S)-4-(2-chloro-4-fluorophenyl)-7-(2-methyl-3-oxo-3-(piperidin-1-yl)propyl)-2H-chromen-2-one, Example 18 and (R)-4-(2-chloro-4-fluorophenyl)-7-(2-methyl-3-oxo-3-(piperidin-1-yl)propyl)-2H-chromen-2-one, Example 19 [Step 2]: To a stirred solution of 3-(4-(2-chloro-4-fluorophenyl)-2-oxo-2H-chromen-7-yl)-2-methylpropanoic acid (Example 17, 140 mg, 0.4 mmol) in CH₂C₁₂ (3 mL) at ambient temperature was added DIPEA (0.2 mL, 1.2 mmol), piperidine (0.05 mL, 0.5 mmol) and T₃P (0.2 mL, 0.4 mmol). The reaction mixture was stirred for 16 h., then diluted with EtOAc, washed with water, then brine, dried over anhydrous sodium sulfate, filtered, and concentrated under reduced pressure. The product was purified by flash column chromatography on silica gel to afford 4-(2-chloro-4-fluorophenyl)-7-(2-methyl-3-oxo-3-(piperidin-1-yl)propyl)-2H-chromen-2-one (110 mg). This racemic mixture was further separated by RP chiral prep-HPLC to afford(S)-4-(2-chloro-4-fluorophenyl)-7-(2-methyl-3-oxo-3-(piperidin-1-yl)propyl)-2H-chromen-2-one (Example 18, 25 mg) and (R)-4-(2-chloro-4-fluorophenyl)-7-(2-methyl-3-oxo-3-(piperidin-1-yl)propyl)-2H-chromen-2-one (Example 19, 25 mg).

Analysis of (S)-4-(2-chloro-4-fluorophenyl)-7-(2-methyl-3-oxo-3-(piperidin-1-yl)propyl)-2H-chromen-2-one (Example 18): LCMS: Calcd. for C₂₄H₂₃ClFNO₃: 427: found: [M+H]⁺=428. ¹H NMR (400 MHz, DMSO-do at 100° C.) δ 7.61-7.52 (m, 2H), 7.41-7.37 (m, 1H), 7.31-7.28 (m, 1H), 7.12-6.93 (m, 2H), 6.36 (s, 1H), 3.41-3.35 (m, 4H), 3.19-3.17 (m, 1H), 2.74-2.67 (m, 1H), 1.52 (br s, 2H), 1.34-1.27 (m, 5H), 1.06 (d, 3H).

Analysis of (R)-4-(2-chloro-4-fluorophenyl)-7-(2-methyl-3-oxo-3-(piperidin-1-yl)propyl)-2H-chromen-2-one (Example 19): LCMS (ESI): Calcd. for C₂₄H₂₃ClFNO₃: 427, found: [M+H]⁺=428. ¹H NMR (400 MHz, DMSO-d₆) δ 7.71 (d, 1H), 7.56 (dd, 1H), 7.46-7.42 (m, 1H), 7.37-7.31 (m, 1H), 7.16 (dd, 1H), 6.94 (dd, 1H), 6.44 (s, 1H), 3.50-3.48 (m, 1H), 3.37-3.36 (m, 1H), 3.20 (br s, 2H), 2.97-2.90 (m, 1H), 2.71-2.67 (m, 1H), 1.47-1.45 (m, 2H), 1.36-1.34 (m, 2H), 1.26-1.11 (m, 3H), 1.02 (d, 3H).

Synthesis of (S)-3-(4-(2-chloro-4-fluorophenyl)-2-oxo-2H-chromen-7-yl)-N,N,2-trimethylpropanamide, Example 20 and (R)-3-(4-(2-chloro-4-fluorophenyl)-2-oxo-2H-chromen-7-yl)-N,N,2-trimethylpropanamide, Example 21 [Step 3]: To a stirred solution of 3-(4-(2-chloro-4-fluorophenyl)-2-oxo-2H-chromen-7-yl)-2-methylpropanoic acid (Example 17, 230 mg, 0.6 mmol) in in CH₂Cl₂ (3 mL) at ambient temperature was added TEA (0.4 mL, 3.2 mol), dimethylamine HCl (105 mg, 1.3 mmol), T₃P (0.3 mL, 1 mmol). The reaction mixture stirred for 16 h., then diluted with EtOAc, washed with water, then brine, dried over anhydrous sodium sulfate, filtered, and concentrated under reduced pressure. The product was purified by flash column chromatography on silica gel to afford 3-(4-(2-chloro-4-fluorophenyl)-2-oxo-2H-chromen-7-yl)-N,N,2-trimethylpropanamide (74 mg). The racemic compound was separated by RP chiral prep-HPLC to afford(S)-3-(4-(2-chloro-4-fluorophenyl)-2-oxo-2H-chromen-7-yl)-N,N,2-trimethylpropanamide (Example 20, 33 mg) and (R)-3-(4-(2-chloro-4-fluorophenyl)-2-oxo-2H-chromen-7-yl)-N,N,2-trimethylpropanamide (Example 21, 25 mg).

Analysis of (S)-3-(4-(2-chloro-4-fluorophenyl)-2-oxo-2H-chromen-7-yl)-N,N,2-trimethylpropanamide (Example 20): LCMS: Calcd. for C₂₁H₁₉ClFNO₃: 387, found: [M+H]⁺=388. ¹H NMR (400 MHz, DMSO-d₆) δ 7.71 (dd, 1H), 7.59 (t, 1H), 7.46-7.42 (m, 1H), 7.35 (s, 1H), 7.14 (t, 1H), 6.94 (d, 1H), 6.45 (s, 1H), 3.17 (q, 1H), 2.98-2.91 (m, 4H), 2.76 (s, 3H), 2.66 (dd, 1H), 1.01 (d, 3H).

Analysis of (R)-3-(4-(2-chloro-4-fluorophenyl)-2-oxo-2H-chromen-7-yl)-N,N,2-trimethylpropanamide (Example 21): LCMS: Calcd. for C₂₁H₁₉ClFNO₃: 387, found: [M+H]⁺=388. ¹H NMR (400 MHz, DMSO-d₆) δ 7.71 (dd, 1H), 7.59 (t, 1H), 7.46-7.42 (m, 1H), 7.35 (s, 1H), 7.14 (t, 1H), 6.94 (d, 1H), 6.45 (s, 1H), 3.17 (q, 1H), 2.97-2.91 (m, 4H), 2.76 (s, 3H), 2.68 (dd, 1H), 1.00 (d, 3H).

### Examples 22 and 23: Synthesis of 4-(2-chloro-4-fluorophenyl)-2-oxo-N-propyl-2H-chromene-7-carboxamide and 4-(2-chloro-4-fluorophenyl)-N-ethyl-N-methyl-2-oxo-2H-chromene-7-carboxamide

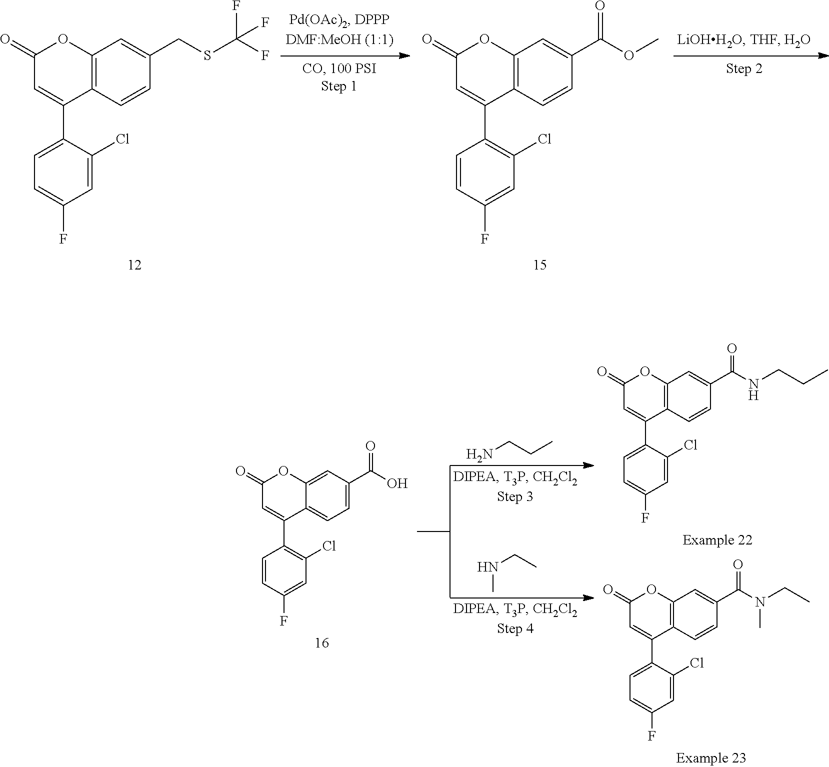

Synthesis of methyl 4-(2-chloro-4-fluorophenyl)-2-oxo-2H-chromene-7-carboxylate, 15 [Step 1]: To an oven-dried Parr-autoclave vessel was added 4-(2-chloro-4-fluorophenyl)-2-oxo-2H-chromen-7-yl trifluoromethane sulfonate (12, 500 mg, 1 mmol) in DMF (4 mL) and methanol (4 mL), DPPP (50 mg, 0.1 mmol), and palladium acetate (25 mg, 0.1 mmol). The reaction vessel was closed and pressurized to 100 PSI with CO. The vessel was heated to 65° C. for 16 h. then cooled to ambient temperature. The reaction mixture was filtered through a celite pad and the filtrated concentrated under reduced pressure. The resulting residue was partitioned between EtOAc and water. The organic layer was collected, washed with water, then brine, dried over anhydrous sodium sulfate, filtered, and concentrated under reduced pressure. The product was purified by flash column chromatography on silica gel to afford methyl 4-(2-chloro-4-fluorophenyl)-2-oxo-2H-chromene-7-carboxylate (15, 340 mg). LCMS (ESI) Calcd. for C₁₇H₁₀ClFO₄: 332, found: [M+H]⁺=333. ¹H NMR (400 MHz, DMSO-d₆) δ 7.94 (d, 1H), 7.84 (dd, 1H), 7.75 (dd, 1H), 7.61 (dd, 1H), 7.50-7.46 (m, 1H), 7.21 (d, 1H), 6.70 (s, 1H), 3.90 (s, 3H).

Synthesis of 4-(2-chloro-4-fluorophenyl)-2-oxo-2H-chromene-7-carboxylic acid, 16 [Step 2]: To a solution of methyl 4-(2-chloro-4-fluorophenyl)-2-oxo-2H-chromene-7-carboxylate (15, 340 mg, 1 mmol) in THF (10 mL) and water (4 mL) at ambient temperature was added LiOH. H₂O (130 mg, 3 mmol). The resulting mixture was stirred for 4 h., then concentrated under reduced pressure. The resulting mixture was dissolved in water and extracted with EtOAc. The aqueous layer was separated, acidified with 1N aqueous HCl solution until pH decreased to 3, then extracted with EtOAc. The combined organic extracts were washed with brine, dried over anhydrous sodium sulfate, filtered, and concentrated under reduced pressure to afford 4-(2-chloro-4-fluorophenyl)-2-oxo-2H-chromene-7-carboxylic acid (16, 330 mg). LCMS (ESI) Calcd. for C₁₆H₈ClFO₄: 318, found: [M+H]⁺=320. ¹H NMR (400 MHz, DMSO-d₆) δ 13.49 (s, 1H), 7.90 (d, 1H), 7.82 (dd, 1H), 7.74 (dd, 1H), 7.61 (dd, 1H), 7.49-7.45 (m, 1H), 7.18 (d, 1H), 6.68 (s, 1H).

Synthesis of 4-(2-chloro-4-fluorophenyl)-2-oxo-N-propyl-2H-chromene-7-carboxamide, Example 22 [Step 3]: To a stirred solution of 4-(2-chloro-4-fluorophenyl)-2-oxo-2H-chromene-7-carboxylic acid (16, 160 mg, 0.5 mmol) in CH₂Cl₂ (5 mL) at ambient temperature was added DIPEA (0.2 mL, 1.5 mmol) followed by N-ethylmethylamine (0.1 mL, 1.5 mmol). The mixture was cooled to 0° C. and T₃P, 50% (0.2 mL, 0.6 mmol) was added. The mixture was then allowed to warm to ambient temperature and stirred for 16 h. The reaction mixture was diluted with water and extracted with CH₂Cl₂. The combined organic extracts were washed with water, then brine, dried over anhydrous sodium sulfate, filtered, and concentrated under reduced pressure. The product was purified by flash column chromatography on silica gel, and then by reverse phase prep-HPLC and lyophilization to afford 4-(2-chloro-4-fluorophenyl)-2-oxo-N-propyl-2H-chromene-7-carboxamide (Example 22, 80 mg). LCMS (ESI) Calcd. For C₁₉H₁₅ClFNO₃: 359, found: [M−H]⁺=358. ¹H NMR (400 MHz, DMSO-d₆) δ 8.69 (t, 1H), 7.90 (d, 1H), 7.75-7.72 (m, 2H), 7.63-7.59 (m, 1H), 7.47 (dt, 1H), 7.14 (d, 1H), 6.62 (s, 1H), 3.26-3.22 (m, 2H), 1.57-1.50 (m, 2H), 0.89 (t, 3H).

Synthesis of 4-(2-chloro-4-fluorophenyl)-N-ethyl-N-methyl-2-oxo-2H-chromene-7-carboxamide, Example 23 [Step 4]: To a stirred solution of 4-(2-chloro-4-fluorophenyl)-2-oxo-2H-chromene-7-carboxylic acid (16, 160 mg, 0.5 mmol) in CH₂Cl₂ (5 mL) at ambient temperature was added DIPEA (0.2 mL, 1.5 mmol) followed by N-propylamine (90 mg, 1.5 mmol). The mixture was cooled to 0° C. and T₃P, 50% (0.2 mL, 0.6 mmol) was added. The resulting mixture was allowed to warm up to ambient temperature and stirred for 16 h. The reaction mixture was then diluted with water and extracted with CH₂Cl₂. The combined organic extracts were washed with water, then brine, dried over anhydrous sodium sulfate, filtered, and concentrated under reduced pressure. The product was purified by flash column chromatography on silica gel, and then by reverse phase prep-HPLC and lyophilization to afford 4-(2-chloro-4-fluorophenyl)-N-ethyl-N-methyl-2-oxo-2H-chromene-7-carboxamide (Example 23, 90 mg). LCMS (ESI) Calcd. for C₁₉H₁₅ClFNO: 359, found: [M+H]⁺=360. ¹H NMR (400 MHz, DMSO-d₆ at 100° C.) δ 7.64-7.57 (m, 2H), 7.45-7.39 (m, 2H), 7.27 (d, 1H), 7.11 (d, 1H), 6.51 (s, 1H), 3.37 (br s, 2H), 2.96 (d, 3H), 1.13 (t, 3H).

### Examples 24 and 25: Synthesis of ethyl (4-(2-chloro-4-fluorophenyl)-2-oxo-2H-chromen-7-yl)(methyl)carbamate and 4-(2-chloro-4-fluorophenyl)-7-(methylamino)-2H-chromen-2-one

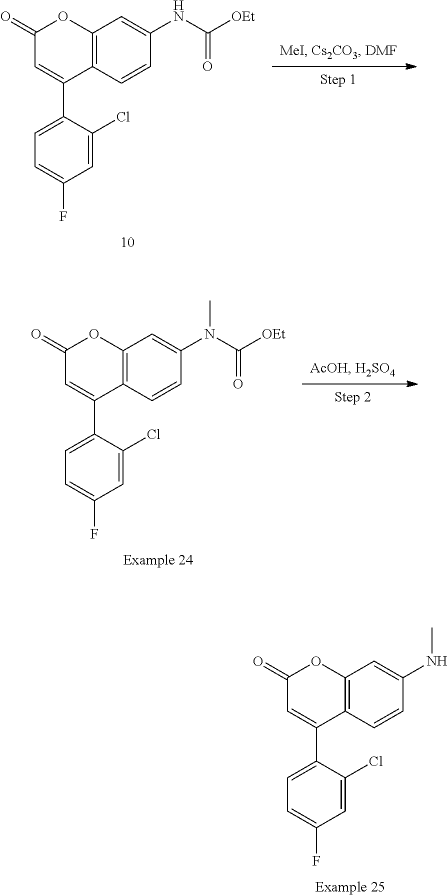 [SMILES: CCOC(=O)Nc1ccc2c(-c3ccc(F)cc3Cl)cc(=O)oc2c1 ; CCOC(=O)N(C)c1ccc2c(-c3ccc(F)cc3Cl)cc(=O)oc2c1 ; CNc1ccc2c(-c3ccc(F)cc3Cl)cc(=O)oc2c1]

Synthesis of ethyl (4-(2-chloro-4-fluorophenyl)-2-oxo-2H-chromen-7-yl)(methyl)carbamate, Example 24 [Step 1]: To a stirred solution of ethyl N-[4-(2-chloro-4-fluoro-phenyl)-2-oxo-chromen-7-yl]carbamate (10, 650 mg, 1.8 mmol) in DMF (8 mL) at ambient temperature was added Cs₂CO₃ (1.5 g, 4.5 mmol). The mixture was cooled to 0° C. and methyl iodide (2.2 mL, 36 mmol) was added dropwise. The reaction mixture was warmed to ambient temperature and stirred for 3 h. The reaction mixture was then diluted with EtOAc, washed with water, then brine, dried over anhydrous sodium sulfate, filtered, and concentrated under reduced pressure. The product was purified by flash column chromatography on silica gel to give ethyl (4-(2-chloro-4-fluorophenyl)-2-oxo-2H-chromen-7-yl)(methyl)carbamate (Example 24, 550 mg). ¹H NMR (400 MHz, chloroform-d) δ 7.36-7.23 (m, 3H), 7.21-7.11 (m, 2H), 7.00 (d, 1H), 6.29 (s, 1H), 4.22 (q, 2H), 1.28 (t, 3H). LCMS: Calcd. for C₁₉H₁₅ClFNO₄: 375, found [M+H]⁺=376.

Synthesis of 4-(2-chloro-4-fluorophenyl)-7-(methylamino)-2H-chromen-2-one, Example 25 [Step 2]: Ethyl (4-(2-chloro-4-fluorophenyl)-2-oxo-2H-chromen-7-yl)(methyl)carbamate (Example 24, 550 mg, 1.5 mmol) was added to sulfuric acid (4.5 mL) at 0° C. with constant stirring. Acetic acid (1 mL, 17 mmol) was added dropwise and the mixture heated to 120° C. for 4 h. The mixture was then cooled to ambient temperature, neutralized with saturated aqueous sodium bicarbonate solution, and extracted with EtOAc. The combined organic extracts were washed with water, then brine, dried over anhydrous sodium sulfate, filtered, and concentrated under reduced pressure. The product was purified by flash column chromatography on silica gel followed by reverse phase prep-HPLC and lyophilization to afford 4-(2-chloro-4-fluorophenyl)-7-(methylamino)-2H-chromen-2-one (Example 25, 300 mg, 67%). 1H NMR (400 MHz, DMSO-d₆) δ 7.66 (dd, 1H), 7.52 (td, 1H), 7.45-7.35 (td, 1H), 6.83 (m, 1H), 6.69 (d, 1H), 6.51 (dd, 1H), 6.44 (m, 1H), 5.93 (s, 1H), 2.75 (d, 3H). LCMS: Calcd. for C₁₆H₁₁ClFNO₂: 303, found [M+H]⁺=304.

### Examples 26-28: Synthesis of (R)-ethyl 2-((4-(2-chloro-4-fluorophenyl)-2-oxo-2H-chromen-7-yl)amino)propanoate, (R)-4-(2-chloro-4-fluorophenyl)-7-((1-oxo-1-(piperidin-1-yl)propan-2-yl)amino)-2H-chromen-2-one, and (R)-2-((4-(2-chloro-4-fluorophenyl)-2-oxo-2H-chromen-7-yl)amino)-N,N-dimethylpropanamide

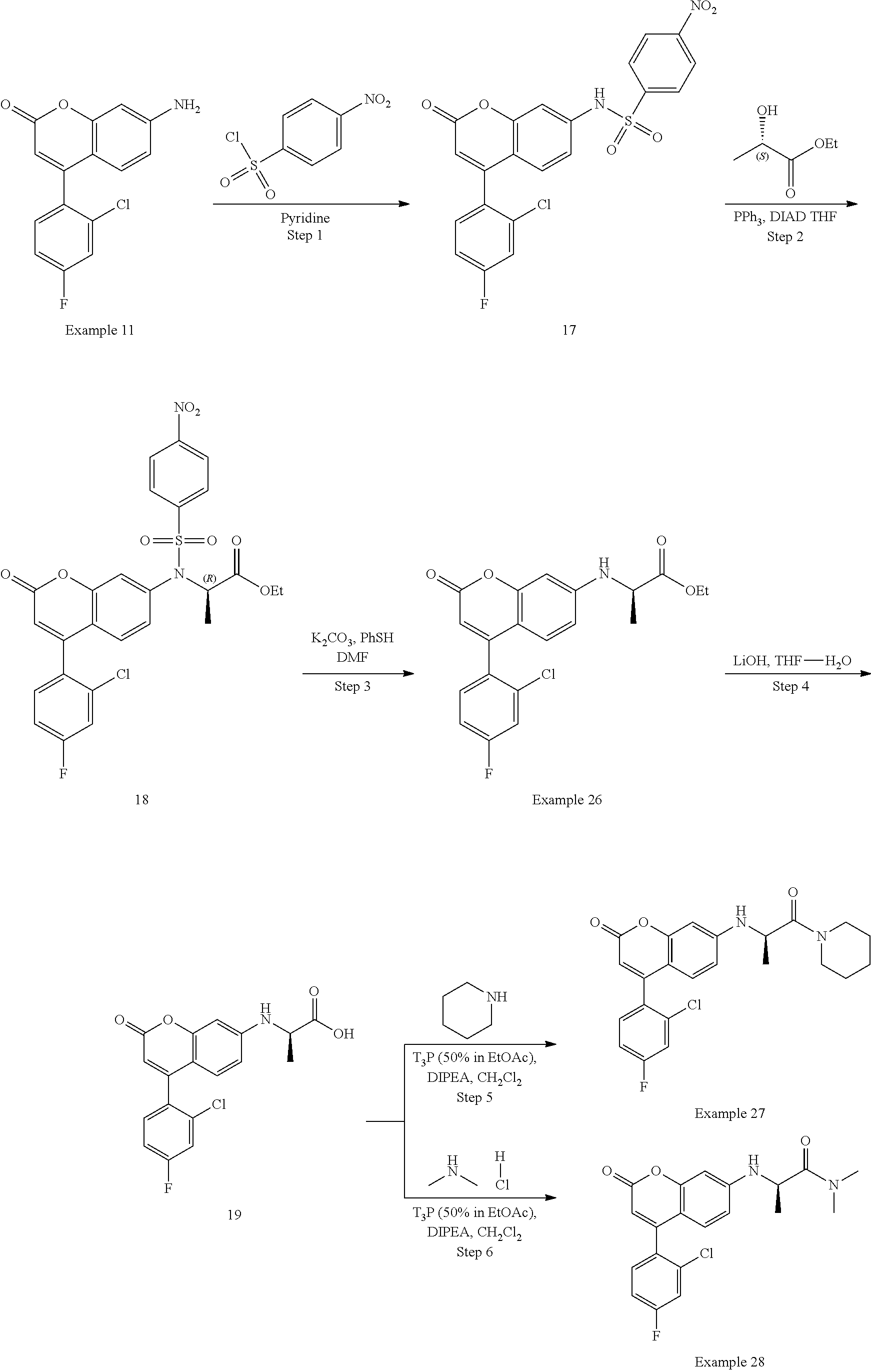

Synthesis of N-(4-(2-chloro-4-fluorophenyl)-2-oxo-2H-chromen-7-yl)-4-nitrobenzenesulfonamide, 17 [Step 1]: 4-Nitrophenylsulfonyl chloride (1.5 g, 6.9 mmol) was added to a solution of 7-amino-4-(2-chloro-4-fluorophenyl)-2H-chromen-2-one (Example 11, 1 g, 3.4 mmol) in pyridine (10 mL) and the mixture stirred at ambient temperature for 1 h. The reaction mixture was then diluted with water and extracted with EtOAc (twice). The combined organic extracts were washed with brine, dried over anhydrous sodium sulfate, filtered, and concentrated under reduced pressure. The product was purified by flash column chromatography on silica gel to afford N-(4-(2-chloro-4-fluorophenyl)-2-oxo-2H-chromen-7-yl)-4-nitrobenzenesulfonamide (17, 700 mg). LCMS: (ESI) Calcd. for C₂₀H₁₂ClFN₃O₆S: 474, found: [M+H]⁺=475. ¹H NMR (400 MHz, DMSO-d₆) δ 11.36 (s, 1H), 8.40 (d, 2H), 8.12 (d, 2H), 7.69 (dd, 1H), 7.52 (br s, 1H), 7.43-7.38 (m, 1H), 7.18 (s, 1H), 7.05 (d, 1H), 6.96 (d, 1H), 6.37 (s, 1H).

Synthesis of ethyl N-(4-(2-chloro-4-fluorophenyl)-2-oxo-2H-chromen-7-yl)-N-((4-nitrophenyl) sulfonyl)-D-alaninate, 18 [Step 2]: To a stirred solution of N-(4-(2-chloro-4-fluorophenyl)-2-oxo-2H-chromen-7-yl)-4-nitrobenzenesulfonamide (17, 320 mg, 0.7 mmol) and ethyl (S)-2-hydroxypropanoate (0.1 mL, 1.0 mmol) in THF (6 mL) was added PPh₃ (530 mg, 2.0 mmol). The mixture was then cooled to 0° C. and diisopropyl azodicarboxylate (0.4 mL, 2.0 mmol) added. The mixture was heated gradually to 80° C. and stirred at that temperature for 16 h. The reaction mixture was then cooled, diluted with ethyl acetate, washed with water, then brine, dried over anhydrous sodium sulfate, filtered, and concentrated under reduced pressure. The product was purified by flash column chromatography on silica gel to afford ethyl N-(4-(2-chloro-4-fluorophenyl)-2-oxo-2H-chromen-7-yl)-N-((4-nitrophenyl) sulfonyl)-D-alaninate (18, 240 mg). LCMS: Calcd. for C₂₆H₂₀ClFN₂O₈S: 573, found: [M+H]⁺=574.

Synthesis of ethyl (4-(2-chloro-4-fluorophenyl)-2-oxo-2H-chromen-7-yl)-D-alaninate, Example 26 [Step 3]: K₂CO₃ (680 mg, 4.9 mmol) and thiophenol (0.4 mL, 3.6 mmol) were added to a solution of ethyl N-(4-(2-chloro-4-fluorophenyl)-2-oxo-2H-chromen-7-yl)-N-((4-nitrophenyl) sulfonyl)-D-alaninate (18, 700 mg, 1.2 mmol) in DMF (20 mL) at ambient temperature and the mixture stirred for 2 h. The reaction mixture was then diluted with water and extracted with ethyl acetate. The combined organic extracts were washed with brine, dried over anhydrous sodium sulfate, filtered, and concentrated under reduced pressure. The product was purified by flash column chromatography on silica gel and then by reverse phase prep-HPLC and lyophilization to afford ethyl (4-(2-chloro-4-fluorophenyl)-2-oxo-2H-chromen-7-yl)-D-alaninate (Example 26, 200 mg). LCMS: Calcd. for C₂₀H₁₇ClFNO: 388, found: [M+H]⁺=389. ¹H NMR (400 MHz, DMSO-d₆) δ 7.67 (dd, 1H), 7.52 (dd, 1H), 7.42-7.39 (m, 1H), 7.12 (d, 1H), 6.73 (dd, 1H), 6.58-6.54 (m, 1H), 6.46 (br s, 1H), 6.00 (s, 1H), 4.24 (br s, 1H), 4.15 (q, 2H), 1.41 (d, 3H), 1.20 (t, 3H).

Synthesis of (4-(2-chloro-4-fluorophenyl)-2-oxo-2H-chromen-7-yl)-D-alanine, 19 [Step 4]: To a stirred solution of ethyl (4-(2-chloro-4-fluorophenyl)-2-oxo-2H-chromen-7-yl)-D-alaninate (Example 26, 365 mg, 1 mmol) in THF (5 mL) and water (1.5 mL) was added LiOH·H₂O (160 mg, 3.7 mmol). The mixture was stirred for 2 h. and concentrated under reduced pressure. The residue was diluted with water, acidified with citric acid (pH: 4-5), and extracted with ethyl acetate. The combined organic extracts were washed with brine, dried over anhydrous sodium sulfate, filtered, and concentrated under reduced pressure to give (4-(2-chloro-4-fluorophenyl)-2-oxo-2H-chromen-7-yl)-D-alanine (19, 240 mg). LCMS: Calcd. for C₁₈H₁₃ClFNO₄: 360, found: [M+H]⁺=361.

Synthesis of (R)-4-(2-chloro-4-fluorophenyl)-7-((1-oxo-1-(piperidin-1-yl)propan-2-yl)amino)-2H-chromen-2-one, Example 27 [Step 5]: To a stirred solution of (4-(2-chloro-4-fluorophenyl)-2-oxo-2H-chromen-7-yl)-D-alanine (19, 115 mg, 0.3 mmol) in CH₂Cl₂ (5 mL) at 0° were added DIPEA (0.13 mL, 0.9 mmol) followed by piperidine (0.1 mL, 0.9 mmol) and T₃P, 50% in EtOAc (0.3 mL, 0.5 mmol). The resulting mixture was stirred at ambient temperature for 4 h., then concentrated under reduced pressure. The residue was partitioned between EtOAc and water. The organic layer was separated, washed with saturated aqueous NaHCO₃ solution, then brine, dried over anhydrous sodium sulfate, filtered, and concentrated under reduced pressure. The product was purified by reverse phase prep-HPLC and lyophilized to afford (R)-4-(2-chloro-4-fluorophenyl)-7-((1-oxo-1-(piperidin-1-yl)propan-2-yl)amino)-2H-chromen-2-one (Example 27, 24 mg). LCMS: Calcd. for C₂₃H₂₂ClFN₂O₃: 427, found: [M+H]⁺=428. ¹H NMR (400 MHz, DMSO-d₆) δ 7.67 (dd, 1H), 7.53-7.49 (m, 1H), 7.41-7.38 (m, 1H), 6.91 (d, 1H), 6.69 (d, 1H), 6.62 (d, 1H), 6.57 (d, 1H), 5.96 (s, 1H), 4.62 (t, 1H), 3.58-3.35 (m, 4H), 1.61-1.54 (m, 4H), 1.43 (br s, 2H), 1.26 (d, 3H).

Synthesis of (R)-2-((4-(2-chloro-4-fluorophenyl)-2-oxo-2H-chromen-7-yl)amino)-N,N-dimethylpropanamide, Example 28 [Step 6]: To a stirred suspension of dimethylamine hydrochloride (80 mg, 0.9 mmol) in CH₂Cl₂ (5 mL) at 0° C. were added DIPEA (0.2 mL, 0.9 mmol) followed by (4-(2-chloro-4-fluorophenyl)-2-oxo-2H-chromen-7-yl)-D-alanine (19, 115 mg, 0.3 mmol) and T₃P, 50% in EtOAc (0.3 mL, 0.5 mmol). The resulting mixture was stirred at ambient temperature for 4 h. and concentrated under reduced pressure. The residue was partitioned between EtOAc and water. The organic layer was separated, washed with saturated aqueous NaHCO₃ solution, then brine, dried over anhydrous sodium sulfate, filtered, and concentrated under reduced pressure. The product was purified by reverse phase prep-HPLC and lyophilized to afford (R)-2-((4-(2-chloro-4-fluorophenyl)-2-oxo-2H-chromen-7-yl)amino)-N,N-dimethylpropanamide (Example 28, 17 mg). LCMS: Calcd. for C₂₀H₁₈ClFN₂O₃: 388, found: [M+H]⁺=389. ¹H NMR (400 MHz, DMSO-d₆) δ 7.67 (dd, 1H), 7.51 (t, 1H), 7.40 (t, 1H), 6.90 (d, 1H), 6.68 (d, 1H), 6.61 (d, 1H), 6.53 (d, 1H), 5.96 (s, 1H), 4.60-4.57 (m, 1H), 3.11 (s, 3H), 2.85 (s, 3H), 1.27 (d, 3H).

### Examples 29-30: Synthesis of N-(4-(2-methoxyphenyl)-2-oxo-2H-chromen-7-yl)acetamide and N-(4-(2-methoxyphenyl)-2-oxo-2H-chromen-7-yl) isobutyramide

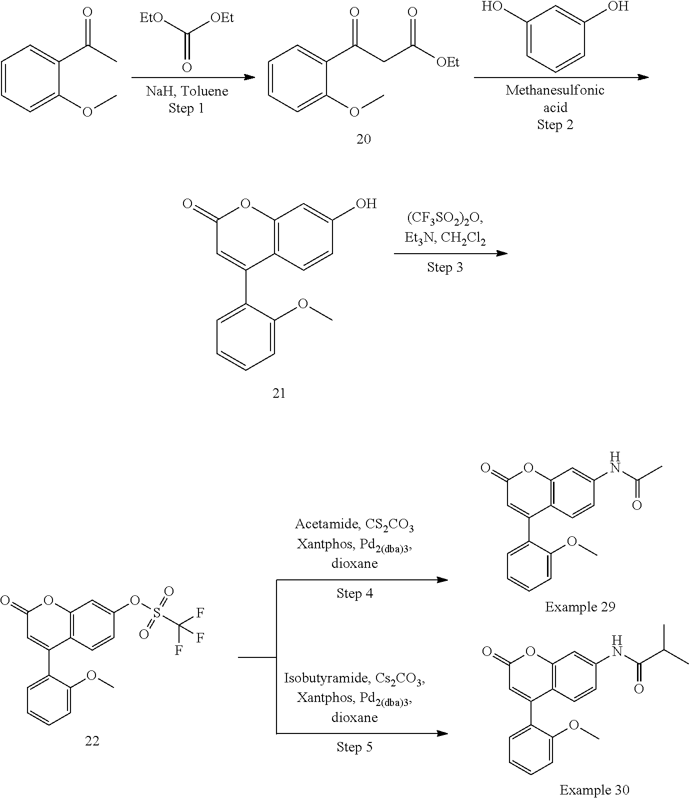

Synthesis of ethyl 3-(2-methoxyphenyl)-3-oxopropanoate, 20 [Step 1]: To a suspension of NaH (60% in oil) (2.9 g, 120 mmol) in toluene (40 ml) was added diethyl carbonate (15.8 g, 130 mmol) at ambient temperature. The mixture was stirred for 15 min., then 1-(2-methoxyphenyl) ethanone (5.0 g, 34.0 mmol) was added. The mixture was gradually warmed to 100° C. and stirred for 4 h. TLC analysis then showed complete conversion of starting material had occurred to a non-polar spot. The reaction mixture was cooled to 0° C. and carefully quenched with saturated aqueous NH₄Cl solution. The mixture was extracted with EtOAc. The combined organic extracts were washed with water, then brine, dried over anhydrous sodium sulfate, filtered, and concentrated under reduced pressure. The product was purified by flash column chromatography on silica gel to afford ethyl 3-(2-methoxyphenyl)-3-oxopropanoate (20, 4.0 g). LCMS (ESI) Calcd. For C₁₂H₁₄O₄: 222, found: [M+H]⁺=223. ¹H NMR (400 MHz, CDCl₃) δ 7.87-7.85 (m, 1H), 7.51-7.47 (m, 1H), 7.02 (t, 1H), 6.96-6.86 (m, 1H), 4.24-4.14 (m, 2H), 3.95 (s, 2H), 3.88 (d, 3H), 1.22 (d, 3H).

Synthesis of 7-hydroxy-4-(2-methoxyphenyl)-2H-chromen-2-one, 21 [Step 2]: Methanesulphonic acid (8.0 mL, 120.0 mmol) was added dropwise to a mixture of ethyl 3-oxo-3-(2-methoxyphenyl)-3-oxo-propanoate (20, 4.0 g, 18.0 mmol) and benzene-1,3-diol (2.0 g, 18.0 mmol) at ambient temperature. The reaction mixture was stirred for 16 h., at which time TLC analysis showed complete conversion of starting material had occurred. The reaction mixture was quenched with ice water. The reaction mixture was filtered, washed with water, then pentane. The product was dried under reduced pressure to give 7-hydroxy-4-(2-methoxyphenyl)-2H-chromen-2-one (21, 4 g). LCMS (ESI) Calcd. For C₁₆H₁₂O₄: 268, found: [M+H]⁺=269. ¹H NMR (400 MHz, DMSO-d₆) δ 10.56 (s, 1H), 7.53-7.49 (m, 1H), 7.27 (dd, 1H), 7.20 (d, 1H), 7.09 (t, 1H), 6.92 (d, 1H), 6.76 (t, 1H), 6.73-6.69 (m, 1H), 6.09, (s, 1H), 3.71 (s, 3H).

Synthesis of 4-(2-methoxyphenyl)-2-oxo-2H-chromen-7-yl trifluoromethanesulfonate, 22 [Step 3]: To a solution of 7-hydroxy-4-(2-methoxyphenyl)-2H-chromen-2-one (21, 1.0 g, 3.8 mmol) in CH₂Cl₂ (15 mL) was added triethylamine (0.8 mL, 5.3 mmol). The mixture was stirred at 0° C. for 10 min., then trifluoromethane sulphonic anhydride (2.6 g, 9 mmol) was added. The reaction mixture was stirred for 12 h. at ambient temperature. After TLC analysis showed reaction completion, the mixture was partitioned between CH₂Cl₂ and water. The organic layer was washed with brine, dried over anhydrous sodium sulfate, filtered, and concentrated under reduced pressure. The product was purified by flash column chromatography on silica gel to afford 2-oxo-4-(2-methoxyphenyl)-2H-chromen-7-yl trifluoromethanesulfonate (22, 700 mg). LCMS (ESI) Calcd. For C₁₇H₁₁F₃O₆ S: 400, found: [M+H]⁺=401. ¹H NMR (400 MHz, DMSO-d₆) δ 7.82 (d, 1H), 7.28 (d, 1H), 7.58-7.54 (m, 1H), 7.41 (dd, 1H), 7.28-7.23 (m, 2H), 7.15-7.12 (m, 1H), 6.51 (s, 1H), 3.73 (s, 3H).

Synthesis of N-(4-(2-methoxyphenyl)-2-oxo-2H-chromen-7-yl)acetamide, Example 29 [Step 4]: To a suspension of 2-oxo-4-(2-methoxyphenyl)-2H-chromen-7-yl trifluoromethanesulfonate (22, 200 mg, 0.5 mmol) in 1,4-dioxane (4 mL) at ambient temperature was added cesium carbonate (330 mg, 0.9 mmol), acetamide (35 mg, 0.6 mmol), and Xantphos (14 mg, 0.025 mmol). The mixture was then degassed with nitrogen for 10 min., and Pd₂(dba)₃ (25 mg, 0.025 mmol) was added. The reaction mixture was then heated to 100° C. for 4 h., when TLC analysis showed complete consumption of starting material had occurred to form a new polar spot. The reaction mixture was cooled to ambient temperature and filtered through a celite bed. The product was washed with ethyl acetate. The combined filtrate was concentrated under reduced pressure. The product was purified by flash column chromatography on silica gel followed by reverse phase prep-HPLC and lyophilization to afford N-(2-oxo-4-(2-methoxyphenyl)-2H-chromen-7-yl)acetamide (Example 29, 30 mg). LCMS (ESI) Calcd. for C₁₈H₁₅NO₄: 309, found: [M+H]⁺=310. ¹H NMR (400 MHz, DMSO-d₆) δ 10.38 (s, 1H), 7.84 (s, 1H), 7.52 (t, 1H), 7.31 (t, 2H), 7.21 (d, 1H), 7.11 (t, 1H), 7.04 (d, 1H), 6.22 (s, 1H), 3.72 (s, 3H), 2.09 (s, 3H).

Synthesis of N-(4-(2-methoxyphenyl)-2-oxo-2H-chromen-7-yl) isobutyramide, Example 30 [Step 5]: To a suspension of 2-oxo-4-(2-methoxyphenyl)-2H-chromen-7-yl trifluoromethanesulfonate (22, 200 mg, 0.50 mmol), in dioxane (4 mL) at ambient temperature was added cesium carbonate (330 mg, 0.99 mmol), isobutyramide, (50 mg, 0.6 mmol), and Xantphos (15 mg, 0.05 mmol). The mixture was degassed with nitrogen for 10 min., then Pd₂(dba)₃ was added (25 mg, 0.025 mmol). The reaction mixture was then heated to 100° C. for 4 h., when TLC analysis showed full consumption of starting material had occurred to form a new polar spot. The reaction mixture was cooled to ambient temperature, then filtered through a celite bed. The product was washed with ethyl acetate. The combined filtrate was concentrated under reduced pressure. The product was purified by flash column chromatography on silica gel followed by reverse phase prep-HPLC and lyophilization to afford N-(4-(2-methoxyphenyl)-2-oxo-2H-chromen-7-yl) isobutyramide (Example 30, 50 mg). LCMS (ESI) Calcd. for C₂₀H₁₉NO₄: 337, found: [M+H]⁺=338. ¹H NMR (400 MHz, DMSO-d₆) δ 10.27 (s, 1H), 7.87 (br s, 1H), 7.53 (t, 1H), 7.38 (d, 1H), 7.30 (d, 1H), 7.22 (d, 1H), 7.11 (t, 1H), 7.04 (d, 1H), 6.22 (s, 1H), 3.72 (s, 3H), 2.67-2.60 (m, 1H), 1.14-1.10 (t, 6H).

### Example 31: Synthesis of 4-(2-chloro-4-fluorophenyl)-7-(dimethylamino)-2H-chromen-2-one

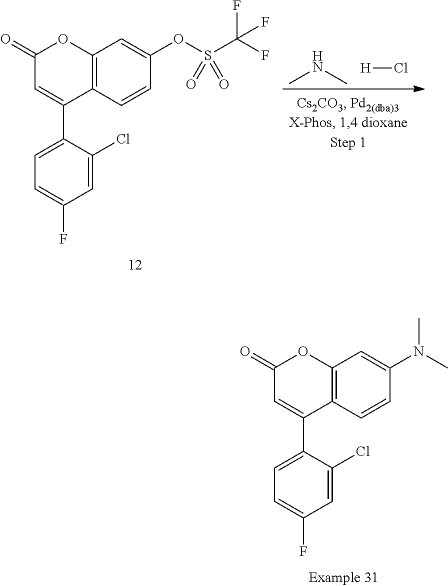

Synthesis of 4-(2-chloro-4-fluorophenyl)-7-(dimethylamino)-2H-chromen-2-one, Example 31 [Step 1]: To a stirred solution of 4-(2-chloro-4-fluorophenyl)-2-oxo-2H-chromen-7-yl trifluoromethanesulfonate (12, 130 mg, 0.3 mmol) in 1,4-dioxane (2 mL) was added dimethylamine hydrochloride (50 mg, 0.6 mmol) followed by Cs₂CO₃ (200 mg, 0.6 mmol). The mixture was degassed with nitrogen for 5 min. in a tube, then Pd₂(dba)₃ (28 mg, 0.03 mmol) added, followed by X-Phos (30 mg, 0.1 mmol). The tube was then sealed and heated to 100° C. for 16 h. The reaction mixture was cooled, filtered, diluted with EtOAc, washed with water, then brine, dried over anhydrous sodium sulfate, filtered, and concentrated under reduced pressure. The product was purified by reverse phase prep-HPLC to afford 4-(2-chloro-4-fluorophenyl)-7-(dimethylamino)-2H-chromen-2-one (Example 31, 20 mg). LCMS: Calcd. for C₁₇H₁₃ClFNO₂: 317, found: [M+H]⁺=318. ¹H NMR (400 MHz, DMSO-d₆) δ 7.68 (dd, 1H), 7.54 (dd, 1H), 7.43-7.38 (m, 1H), 6.78 (d, 1H), 6.71-6.64 (m, 2H), 5.99 (s, 1H), 3.02 (s, 6H).

### Examples 32-33: Synthesis of N-(2-oxo-4-(o-tolyl)-2H-chromen-7-yl)acetamide and N-(2-oxo-4-(o-tolyl)-2H-chromen-7-yl) isobutyramide

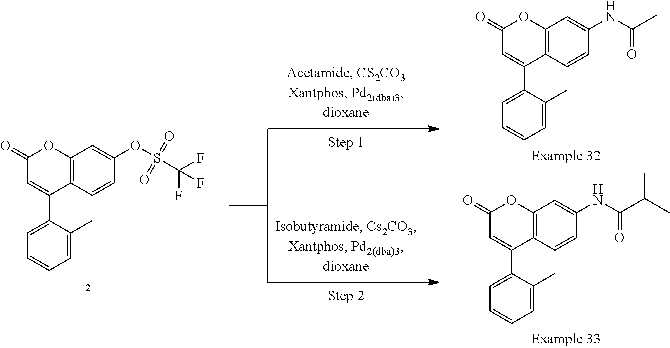 [SMILES: Cc1ccccc1-c1cc(=O)oc2cc(OS(=O)(=O)C(F)(F)F)ccc12 ; Cc1ccccc1-c1cc(=O)oc2cc(NC(=O)C(C)C)ccc12 ; CC(=O)Nc1ccc2c(-c3ccccc3C)cc(=O)oc2c1]

Synthesis of N-(2-oxo-4-(o-tolyl)-2H-chromen-7-yl)acetamide, Example 32 [Step 1]: To a suspension of 2-oxo-4-(o-tolyl)-2H-chromen-7-yl trifluoromethanesulfonate (2, 250 mg, 0.6 mmol) in 1,4-dioxane (4 mL) at ambient temperature was added cesium carbonate (400 mg, 1.3 mmol), acetamide (50 mg, 0.8 mmol), and Xantphos (20 mg, 0.02 mmol). The mixture was degassed with nitrogen for 10 min., then Pd₂(dba)₃ (30 mg, 0.03 mmol) added. The reaction mixture was heated at 100° C. for 4 h., when TLC analysis showed complete consumption of starting material had occurred to form a new polar spot. The reaction mixture was cooled to ambient temperature and filtered through a celite bed. The product was washed with ethyl acetate. The combined filtrate was concentrated under reduced pressure and the product purified by flash column chromatography on silica gel to afford N-(2-oxo-4-(o-tolyl)-2H-chromen-7-yl)acetamide (Example 32, 70 mg). LCMS (ESI) Calcd. for C₁₈H₁₅NO₃: 293, found: [M+H]⁺=294. ¹H NMR (400 MHz, DMSO-d₆) δ 10.42 (s, 1H), 7.88 (d, 1H), 7.43-7.32 (m, 4H), 7.24 (d, 1H), 6.91 (d, 1H), 6.22 (s, 1H), 2.10 (d, 6H).

Synthesis of N-(2-oxo-4-(o-tolyl)-2H-chromen-7-yl) isobutyramide, Example 33 [Step 2]: To a suspension of 2-oxo-4-(o-tolyl)-2H-chromen-7-yl trifluoromethanesulfonate (2, 250 mg, 0.6 mmol) in 1,4-dioxane (4 mL) at ambient temperature was added cesium carbonate (400 mg, 1.3 mmol), isobutyramide (70 mg, 0.8 mmol), and Xantphos (14 mg, 0.03 mmol). The mixture was degassed with nitrogen for 10 min., then Pd₂(dba)₃ was added (30 mg, 0.03 mmol). The reaction mixture was heated at 100° C. for 4 h., when TLC analysis showed complete consumption of starting material had occurred to form a new polar spot. The reaction mixture was cooled to ambient temperature and filtered through a celite bed. The product was washed with ethyl acetate. The combined filtrate was concentrated under reduced pressure and the product was purified by flash column chromatography on silica gel, followed by reverse phase prep-HPLC and lyophilization to afford N-(2-oxo-4-(o-tolyl)-2H-chromen-7-yl) isobutyramide (Example 33, 90 mg). LCMS (ESI) Calcd. for C₂₀H₁₉NO₃: 321, found: [M+H]⁺=322. ¹H NMR (400 MHz, DMSO-d₆) δ 10.30 (s, 1H), 7.91 (s, 1H), 7.43-7.35 (m, 4H), 7.24 (d, 1H), 6.91 (d, 1H), 6.21 (s, 1H), 2.49 (s, 1H), 2.10 (s, 3H), 1.11 (d, 6H).

### Examples 34-37: Synthesis of N,N-dimethyl-2-oxo-4-(o-tolyl)-2H-chromene-7-carboxamide, 2-oxo-N-phenyl-4-(o-tolyl)-2H-chromene-7-carboxamide, N-cyclopentyl-2-oxo-4-(o-tolyl)-2H-chromene-7-carboxamide, and 7-(piperidine-1-carbonyl)-4-(o-tolyl)-2H-chromen-2-one

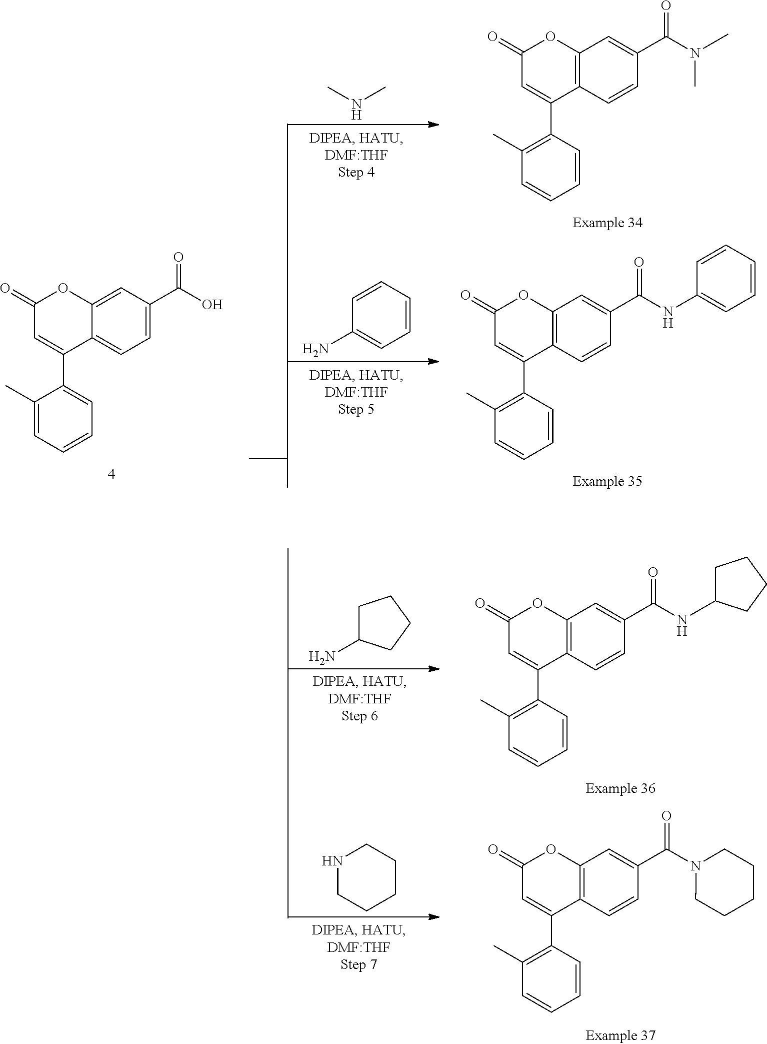

Synthesis of N,N-dimethyl-2-oxo-4-(o-tolyl)-2H-chromene-7-carboxamide, Example 34 [Step 4]: To a solution of HATU (305 mg, 0.8 mmol) in THF (1 mL) and DMF (1 mL) at ambient temperature was added 2-oxo-4-(o-tolyl)-2H-chromene-7-carboxylic acid (4, 150 mg, 0.5 mmol) followed by N,N-diisopropylethylamine (0.3 mL, 1.6 mmol). The reaction mixture was stirred at ambient temperature for 15 minutes, then 2M dimethylamine in THF (0.3 mL, 5.4 mmol) was added. The resulting mixture stirred at ambient temperature for 16 h., then diluted with EtOAc. The mixture was washed with 5% aqueous K₂CO₃ solution, water, then brine, dried over anhydrous sodium sulfate, filtered, and concentrated under reduced pressure. The product was purified by reverse phase prep-HPLC and lyophilization to afford N,N-dimethyl-2-oxo-4-(o-tolyl)-2H-chromene-7-carboxamide (Example 34, 86 mg). LCMS (ESI) Calcd. for C₁₉H₁₇NO₃: 307, found: [M+H]⁺=308. ¹H NMR (400 MHz, DMSO-d₆) δ 7.52 (s, 1H), 7.50-7.32 (m, 3H), 7.28 (d, 2H), 7.02 (d, 1H), 6.46 (s, 1H), 3.00 (s, 3H), 2.90 (s, 3H), 2.13 (s, 3H).

Synthesis of 2-oxo-N-phenyl-4-(o-tolyl)-2H-chromene-7-carboxamide, Example 35 [Step 5]: To a solution of HATU (370 mg, 1.0 mmol) in DMF (1 mL) and THF (1 mL) at ambient temperature was added 2-oxo-4-(o-tolyl)-2H-chromene-7-carboxylic acid (4, 180 mg, 0.6 mmol) followed by N,N-diisopropylethylamine (0.34 mL, 2.0 mmol). The reaction mixture was stirred at ambient temperature for 15 min., then aniline (0.15 mL, 1.6 mmol) was added. The resulting mixture stirred at ambient temperature for 16 h., then diluted with EtOAc. The mixture was washed with 5% aqueous K₂CO₃ solution, water, then brine, dried over anhydrous sodium sulfate, filtered, and concentrated under reduced pressure. The product was purified by reverse phase prep-HPLC and lyophilization to afford 2-oxo-N-phenyl-4-(o-tolyl)-2H-chromene-7-carboxamide (Example 35, 66 mg). LCMS (ESI) Calcd. for C₂₃H₁₇NO₃: 355, found: [M+H]⁺=356. ¹H NMR (400 MHz, DMSO-d₆) δ 10.42 (s, 1H), 8.06 (d, 1H), 7.85-7.74 (m, 3H), 7.53-7.27 (m, 6H), 7.13 (t, 2H), 6.52 (s, 1H), 2.14 (s, 3H).

Synthesis of N-cyclopentyl-2-oxo-4-(o-tolyl)-2H-chromene-7-carboxamide, Example 36 [Step 6]: To a solution of HATU (370 mg, 1.0 mmol) in DMF (1 mL) and THF (1 mL) at ambient temperature was added 2-oxo-4-(o-tolyl)-2H-chromene-7-carboxylic acid (4, 180 mg, 0.6 mmol) followed by N,N-diisopropylethylamine (0.34 mL, 2.0 mmol). The reaction mixture was stirred at ambient temperature for 15 min., then cyclopentanamine (0.16 mL, 1.6 mmol) was added. The resulting mixture stirred at ambient temperature for 16 h., then diluted with EtOAc. The mixture was washed with 5% aqueous K₂CO₃ solution, water, then brine, dried over anhydrous sodium sulfate, filtered, and concentrated under reduced pressure. The product was purified by reverse phase prep-HPLC and lyophilization to afford N-cyclopentyl-2-oxo-4-(o-tolyl)-2H-chromene-7-carboxamide (Example 36, 55 mg). LCMS (ESI) Calcd. for C₂₂H₂₁NO₃: 346, found: [M+H]⁺=347. ¹H NMR (400 MHz, DMSO-d₆) δ 8.49 (d, 1H), 7.92 (d, 1H), 7.71 (dd, 1H), 7.49-7.34 (m, 3H), 7.28 (d, 1H), 7.04 (d, 1H), 6.47 (s, 1H), 4.28-4.16 (m, 1H), 2.11 (s, 3H), 1.98-1.81 (m, 2H), 1.78-1.63 (m, 2H), 1.61-1.47 (m, 4H).

Synthesis of 7-(piperidine-1-carbonyl)-4-(o-tolyl)-2H-chromen-2-one, Example 37 [Step 7]: To a solution of HATU (370 mg, 1.0 mmol) in DMF (1 mL) and THF (1 mL) at ambient temperature was added 2-oxo-4-(o-tolyl)-2H-chromene-7-carboxylic acid (4, 180 mg, 0.6 mmol) followed by N,N-diisopropylethylamine (0.34 mL, 2.0 mmol). The reaction mixture was stirred at ambient temperature for 15 min., then piperidine (0.16 mL, 1.6 mmol) was added. The resulting mixture stirred at ambient temperature for 16 h., then diluted with EtOAc. The mixture was washed with 5% aqueous K₂CO₃ solution, water, then brine, dried over anhydrous sodium sulfate, filtered, and concentrated under reduced pressure. The product was purified by reverse phase prep-HPLC and lyophilization to afford 7-(piperidine-1-carbonyl)-4-(o-tolyl)-2H-chromen-2-one (Example 37, 80 mg). LCMS (ESI) Calcd. for C₂₂H₂₁NO₃: 346, found: [M+H]⁺=347. ¹H NMR (400 MHz, DMSO-d₆) δ 7.52-7.32 (m, 4H), 7.31-7.23 (m, 2H), 7.02 (d, 1H), 6.45 (s, 1H), 3.59 (s, 2H), 3.26 (s, 2H), 2.13 (s, 3H), 1.77-1.28 (m, 6H).

### Example 38: Synthesis of (4-(2-chloro-4-fluorophenyl)-2-oxo-2H-chromen-7-yl)methyl piperidine-1-carboxylate

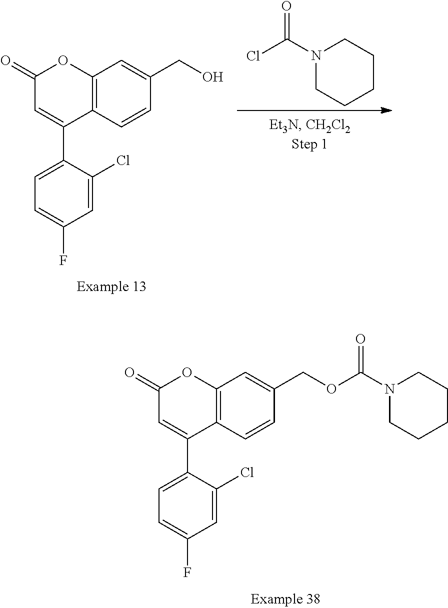 [SMILES: O=c1cc(-c2ccc(F)cc2Cl)c2ccc(CO)cc2o1 ; O=C(Cl)N1CCCCC1 ; O=C(OCc1ccc2c(-c3ccc(F)cc3Cl)cc(=O)oc2c1)N1CCCCC1]

Synthesis of (4-(2-chloro-4-fluorophenyl)-2-oxo-2/I-chromen-7-yl)methyl piperidine-1-carboxylate, Example 38 [Step 1]: To a stirred solution of 4-(2-chloro-4-fluorophenyl)-7-(hydroxymethyl)-2H-chromen-2-one (Example 13, 100 mg, 0.3 mmol) in CH₂Cl₂ (2 mL) at 0° C. was added triethylamine (0.1 mL, 0.8 mmol) followed by piperidine-1-carbonyl chloride (73 mg, 0.5 mmol). The mixture was gradually warmed to ambient temperature and stirred for 16 h. The reaction mixture was then diluted with CH₂Cl₂, washed with water, then brine, dried over anhydrous sodium sulfate, filtered, and concentrated under reduced pressure. The product was purified by reverse phase prep-HPLC to afford (4-(2-chloro-4-fluorophenyl)-2-oxo-2H-chromen-7-yl)methyl piperidine-1-carboxylate (Example 38, 30 mg). LCMS: Calcd. for C₂₂H₁₉ClFNO₄: 415, found: [M+H]⁺: 416. ¹H NMR (400 MHz, DMSO-d₆) δ 7.73 (dd, 1H), 7.60 (m, 1H), 7.47-7.43 (m, 2H), 7.28 (d, 1H), 7.06 (d, 1H), 6.52 (s, 1H), 5.17 (s, 2H), 3.31-3.29 (m, 4H), 1.55-1.46 (m, 6H).

### Example 39: Synthesis of 7-methyl-4-(o-tolyl)-2H-chromen-2-one

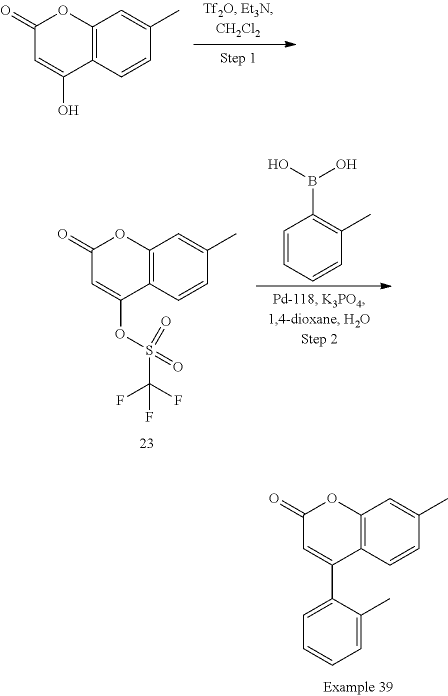

Synthesis of 7-methyl-2-oxo-2H-chromen-4-yl trifluoromethanesulfonate, 23 [Step 1]: To a stirred solution of 4-hydroxy-7-methyl-2H-chromen-2-one (1.0 g, 5.7 mmol) in CH₂Cl₂ (15 mL) at 0° C. was added triethylamine (1.6 mL, 11.4 mmol) followed by trifluoromethanesulfonic anhydride (1.2 mL, 7.4 mmol) dropwise. The mixture was gradually warmed to ambient temperature and stirred overnight. The reaction mixture was then diluted with CH₂Cl₂ and washed with saturated aqueous NaHCO₃ solution, water, then brine, dried over anhydrous sodium sulfate, filtered, and concentrated under reduced pressure. The product was purified by flash column chromatography on silica gel to give 7-methyl-2-oxo-2H-chromen-4-yl trifluoromethanesulfonate (23, 1.0 g). LCMS (ESI) Calcd. for C₁₁H₇F₃O₅S: 308, found: [M+H]⁺=309. ¹H NMR (400 MHz, DMSO-d₆) δ 7.59 (d, 1H), 7.43 (s, 1H), 7.36 (d, 1H), 6.81 (s, 1H), 2.47 (s, 3H).

Synthesis of 7-methyl-4-(o-tolyl)-2H-chromen-2-one, Example 39 [Step 2]: To a stirred solution of (7-methyl-2-oxo-chromen-4-yl)trifluoromethanesulfonate (23, 250 mg, 0.8 mmol) and o-tolylboronic acid (143 mg, 1 mmol) and in 1,4-dioxane (4 mL) and water (1 mL) at ambient temperature was added K₃PO₄ (430 mg, 2.0 mmol). The mixture was degassed with nitrogen for 5 min., then [1,1′-bis(di-tert-butylphosphino) ferrocene]dichloropalladium (II) (53 mg, 0.08 mmol) was added under a nitrogen atmosphere. The mixture was heated to 110° C. for 12 h., then cooled to ambient temperature, and partitioned between EtOAc and water. Organic layer was collected. Aqueous layer was further extracted with EtOAc. Combined organic layers were washed with brine, dried over anhydrous sodium sulfate, filtered, and concentrated under reduced pressure. The product was purified by flash column chromatography on silica gel and lyophilized to give 7-methyl-4-(o-tolyl)-2H-chromen-2-one (Example 39, 140 mg). LCMS (ESI) Calcd. for C₁₇H₁₄O₂: 250, found: [M+H]⁺=251. ¹H NMR (400 MHz, DMSO-d₆) δ 7.46-7.33 (m, 4H), 7.25 (d, 1H), 7.11 (d, 1H), 6.87 (d, 1H), 6.30 (s, 1H), 2.41 (s, 3H), 2.10 (s, 3H).

### Example 40: Synthesis of 4-(2-chloro-4-fluorophenyl)-7-(1-hydroxyethyl)-2H-chromen-2-one

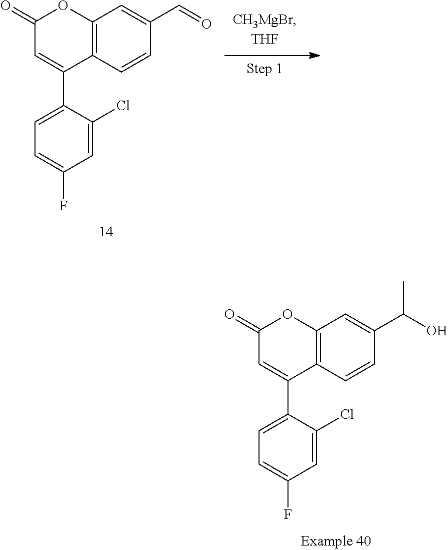 [SMILES: O=Cc1ccc2c(-c3ccc(F)cc3Cl)cc(=O)oc2c1 ; CC(O)c1ccc2c(-c3ccc(F)cc3Cl)cc(=O)oc2c1]

Synthesis of 4-(2-chloro-4-fluorophenyl)-7-(1-hydroxyethyl)-2H-chromen-2-one, Example 40 [Step 1]: To a stirred solution of 4-(2-chloro-4-fluorophenyl)-2-oxo-2H-chromene-7-carbaldehyde (14, 250 mg, 0.8 mmol) in THF (4 mL) at −30° C. and under an argon atmosphere was added methyl magnesium bromide and 3M in diethyl ether (0.3 mL, 0.8 mmol) dropwise. The reaction mixture was stirred for 2 hours, then quenched with saturated aqueous NH₄Cl solution and extracted with EtOAc. The combined organic extracts were washed with brine, dried over anhydrous sodium sulfate, filtered, and concentrated under reduced pressure. The product was purified by flash column chromatography on silica gel then by reverse phase prep-HPLC to afford 4-(2-chloro-4-fluorophenyl)-7-(1-hydroxyethyl)-2H-chromen-2-one (Example 40, 90 mg). LCMS: Calcd. for C₁₇H₁₂ClFO₃: 318, found: [M+H]⁺=319. ¹H NMR (400 MHz, DMSO-d₆) δ 7.71 (dd, 1H), 7.58 (dd, 1H), 7.46-7.43 (m, 2H), 7.31 (t, 1H), 7.00 (dd, 1H), 6.46 (s, 1H), 5.41 (d, 1H), 4.82 (br s, 1H), 1.35 (d, 3H).

### Examples 41-42: Synthesis of chiral analogs of 4-(2-chloro-4-fluorophenyl)-7-(1-ethoxyethyl)-2H-chromen-2-one

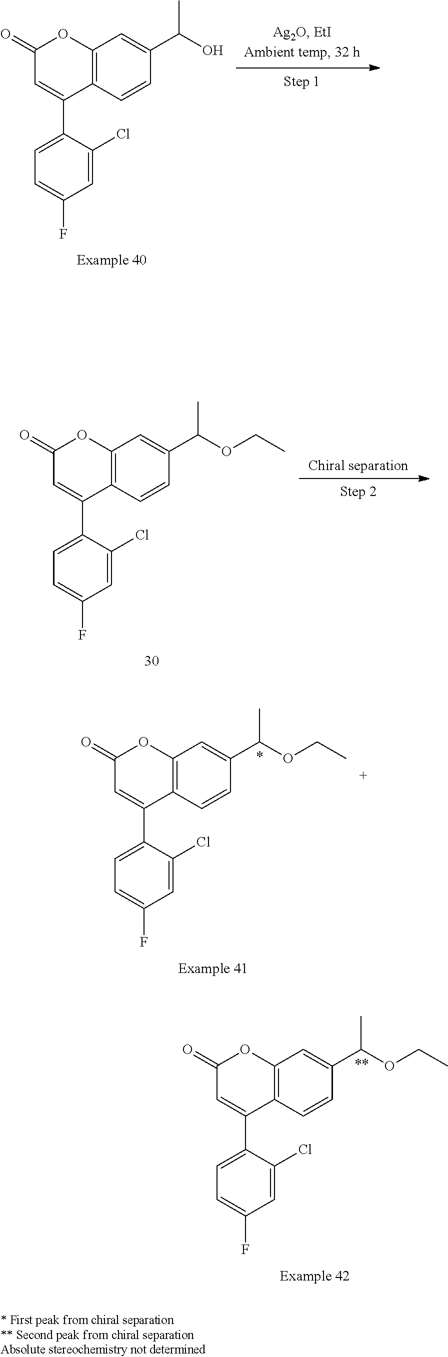 [SMILES: CC(O)c1ccc2c(-c3ccc(F)cc3Cl)cc(=O)oc2c1 ; CCOC(C)c1ccc2c(-c3ccc(F)cc3Cl)cc(=O)oc2c1 ; CCOC(C)c1ccc2c(-c3ccc(F)cc3Cl)cc(=O)oc2c1 ; CCOC(C)c1ccc2c(-c3ccc(F)cc3Cl)cc(=O)oc2c1]

Synthesis 4-(2-chloro-4-fluorophenyl)-7-(1-ethoxyethyl)-2H-chromen-2-one, 30 [Step 1]: To a stirred solution of 4-(2-chloro-4-fluoro-phenyl)-7-(1-hydroxyethyl) chromen-2-one (Example 40, 180 mg, 0.56 mmol) in neat ethyl iodide (2.3 mL, 28 mmol) was added Ag₂O (262 mg, 1.1 mmol) and stirred at ambient temperature for 32 h. The reaction mixture was filtered through a celite bed. The filtrate was washed with saturated aqueous NH₄Cl solution, brine, dried over Na₂SO₄, and concentrated. The product was purified by flash chromatography and then via prep-HPLC purification and lyophilized to afford 4-(2-chloro-4-fluorophenyl)-7-(1-ethoxyethyl)-2H-chromen-2-one (30, 80 mg).

Synthesis of chiral analogs of 4-(2-chloro-4-fluorophenyl)-7-(1-ethoxyethyl)-2H-chromen-2-one, Examples 41 and 42 [Step 2]: 4-(2-chloro-4-fluorophenyl)-7-(1-ethoxyethyl)-2H-chromen-2-one (30, 80 mg) was separated by SFC chiral HPLC purification and lyophilized to afford Peak 1 as 4-(2-chloro-4-fluorophenyl)-7-(1-ethoxyethyl)-2H-chromen-2-one (Example 41, 15 mg) and Peak 2 as 4-(2-chloro-4-fluorophenyl)-7-(1-ethoxyethyl)-2H-chromen-2-one (Example 42, 16 mg). The absolute stereochemistry of these Examples was not determined.

Peak 1:4-(2-chloro-4-fluorophenyl)-7-(1-ethoxyethyl)-2H-chromen-2-one, Example 41: 1H NMR (400 MHz, DMSO-d₆) δ 7.73-7.70 (dd, 1H), 7.60-7.55 (m, 1H), 7.44-7.41 (m, 2H), 7.27 (d, 1H), 7.04 (d, 1H), 6.49 (s, 1H), 4.56 (m, 1H), 3.37 (m, 2H), 1.35 (d, 3H), 1.12 (t, 3H). LCMS: Calcd. for C₁₉H₁₆ClFO₃: 346, found: [M+H]⁺=347.

Peak 2:4-(2-chloro-4-fluorophenyl)-7-(1-ethoxyethyl)-2H-chromen-2-one, Example 42: 1H NMR (400 MHz, DMSO-d₆) δ 7.73-7.70 (dd, 1H), 7.60-7.56 (m, 1H), 7.47-7.41 (m, 2H), 7.27 (d, 1H), 7.04 (d, 1H), 6.49 (s, 1H), 4.56 (q, 1H), 3.39-3.35 (m, 2H), 1.35 (d, 3H), 1.13 (t, 3H). LCMS: Calcd. for C₁₉H₁₆ClFO₃: 346, found: [M+H]⁺=347.

SFC chiral HPLC method: Chiral separation was performed on a Thar SFC-80 instrument using a CHIRALPAK AS-H column (250×21 mm), 5μ, operating at 35° C. with a flow rate of 30 g/min along with co-solvent of 85% CO₂ (at super critical state) and 15% of 0.3% isopropylamine in MeOH, held isocratic and isobaric (ABPR is 100 bar) for up to 14 min. with detection at 274 nm wavelength.

### Example 43: Synthesis 2-oxo-4-(o-tolyl)-2H-chromene-7-carbonitrile

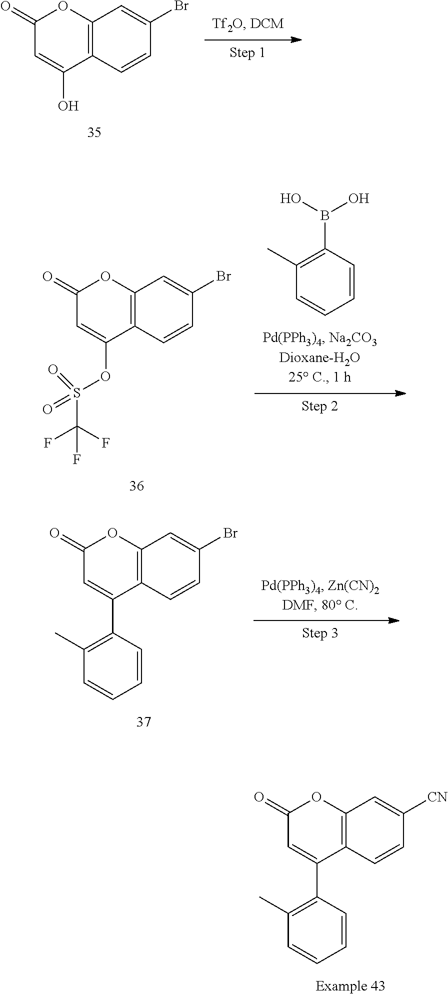

Synthesis of 7-bromo-2-oxo-2H-chromen-4-yl trifluoromethanesulfonate, 36 [Step 1]: To a stirred solution of 7-bromo-4-hydroxy-2H-chromen-2-one (35, 500 mg, 2.1 mmol) in DCM (8 mL) was added triethylamine (0.4 mL, 2.7 mmol) followed by trifluoromethanesulfonic anhydride (0.4 mL, 2.7 mmol) at 0° C. After stirring for 1 h. at 0° C., the reaction mixture was diluted with water, extracted with EtOAc. The combined organic phase was washed with brine, dried over anhydrous Na₂SO₄, filtered and concentrated under reduced pressure. The product purified on a silica gel column using EtOAc/Hexanes to afford 7-bromo-2-oxo-2H-chromen-4-yl trifluoromethanesulfonate (36, 500 mg). LCMS (ESI) Calcd. for C₁₀H₄BrF₃O₅S: 372, found: [M+H]⁺=373. ¹H NMR (400 MHz, DMSO-d₆): δ 7.96-7.94 (d, 1H), 7.75-7.69 (m, 1H), 7.64-7.61 (d, 1H), 6.95 (s, 1H).

Synthesis of 7-bromo-4-(o-tolyl)-2H-chromen-2-one, 37 [Step 2]: An oven dried sealed tube was charged with 7-bromo-2-oxo-2H-chromen-4-yl trifluoromethanesulfonate (36, 200 mg, 0.5 mmol) followed by o-tolylboronic acid (66 mg, 0.5 mmol) and 1,4-dioxane (8 mL). Potassium carbonate (163 mg, 1.2 mmol) in water (2.4 mL) was added to the tube, and the mixture was degassed by bubbling with argon for 10 min. Pd (dppf) C₁₂ (40 mg, 54 μmol) was added, and the reaction mixture was stirred at 25° C. for 1 h. The reaction mixture was partitioned between EtOAc and water. The organic phase was collected, washed with brine, dried over anhydrous Na₂SO₄ and concentrated under reduced pressure. The product was purified by column chromatography over silica gel to afford 7-bromo-4-(o-tolyl)-2H-chromen-2-one (37, 120 mg). LCMS (ESI) Calcd. for C₁₆H₁₁BrO₂: 314, found: [M+H]⁺=315.

Synthesis 2-oxo-4-(o-tolyl)-2H-chromene-7-carbonitrile, Example 43 [Step 3]: An oven dried sealed tube was charged with 7-bromo-4-(o-tolyl)-2H-chromen-2-one (37, 50 mg, 0.2 mmol), DMF (1 mL) and degassed by bubbling with argon for 10 min. Zinc cyanide (20 mg, 0.2 mmol), Pd(PPh₃)₄ (18 mg, 16 μmol) were added, and the reaction mixture was stirred at 80° C. for 12 h. After cooling to ambient temperature, the reaction mixture was quenched with water, extracted with EtOAc. The combined organic phase was washed with cold water, brine, dried over anhydrous Na₂SO₄, filtered and concentrated under reduced pressure. The product was purified by column chromatography over silica gel to afford 2-oxo-4-(o-tolyl)-2H-chromene-7-carbonitrile (Example 43, 18 mg). LCMS (ESI) Calcd. for C₁₇H₁₁NO₂: 261, found: [M+H]⁺=262. ¹H NMR (400 MHz, DMSO-d₆): δ 8.12 (d, 1H), 7.69 (dd, 1H), 7.49-7.35 (m, 3H), 7.27 (d, 1H), 7.11 (d, 1H), 6.60 (s, 1H), 2.11 (s, 3H).

### Example 44: Synthesis of N-methyl-N-(2-oxo-4-(o-tolyl)-2H-chromen-7-yl)acetamide

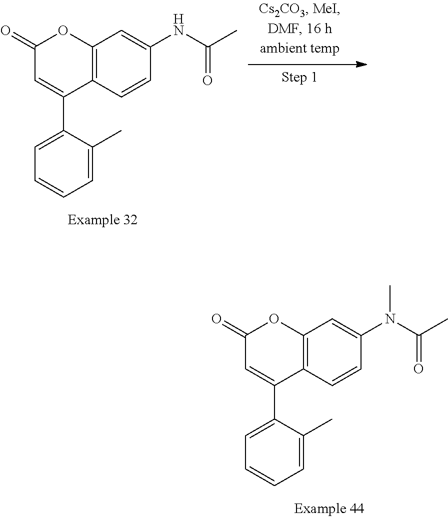 [SMILES: CC(=O)Nc1ccc2c(-c3ccccc3C)cc(=O)oc2c1 ; CC(=O)N(C)c1ccc2c(-c3ccccc3C)cc(=O)oc2c1]

Synthesis of N-methyl-N-(2-oxo-4-(o-tolyl)-2H-chromen-7-yl)acetamide, Example 44 [Step 1]: To a solution of N-methyl-N-(2-oxo-4-(o-tolyl)-2H-chromen-7-yl) acetamide (Example 32, 60 mg, 0.20 mmol) in DMF (4 ml) was added cesium carbonate (130 mg, 0.4 mmol) followed by iodomethane (0.1 ml, 0.7 mmol) at 25° C. and stirred for 16 h. The reaction mixture was partitioned between EtOAc and water. Organic layer was washed with brine dried over NaSO₄ and evaporated under reduced pressure. The product was purified by reverse phase prep-HPLC and lyophilized to afford N-methyl-N-(2-oxo-4-(o-tolyl)-2H-chromen-7-yl)acetamide (Example 44, 10 mg). LCMS (ESI) Calcd. for C₁₉H₁₇NO₃: 307, found: [M+H]⁺=308. ¹H NMR (400 MHz, DMSO-d₆) 7.57 (d, 1H), 7.45-7.35 (m, 3H), 7.28-7.25 (m, 2H), 6.98 (d, 1H), 6.40 (s, 1H), 3.23 (s, 3H), 2.14 (s, 3H), 1.95 (br s, 3H).

### Examples 45 and 46: Synthesis of (R)-4-(2-chloro-4-fluorophenyl)-7-(methyl(1-oxo-1-(piperidin-1-yl)propan-2-yl)amino)-2H-chromen-2-one, (R)-2-((4-(2-chloro-4-fluorophenyl)-2-oxo-2H-chromen-7-yl)(methyl)amino)-N,N-dimethylpropanamide

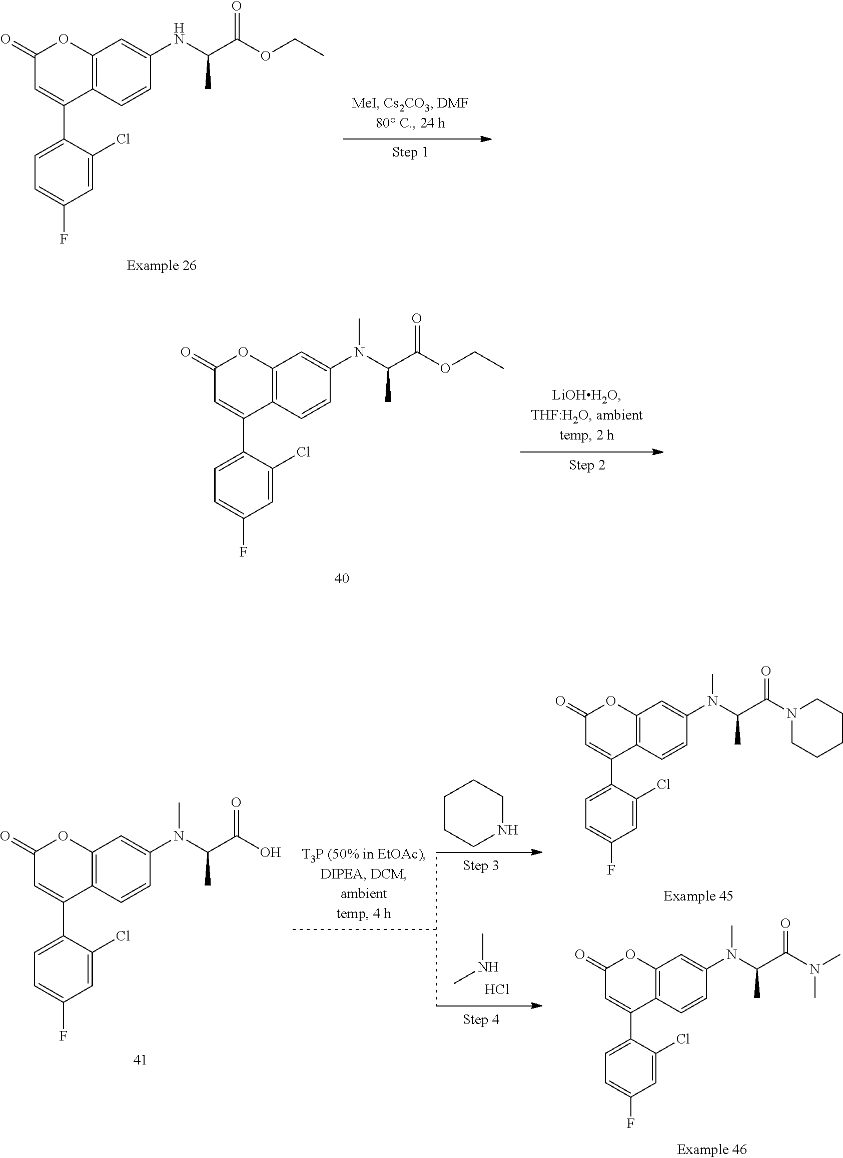

Synthesis of ethyl N-(4-(2-chloro-4-fluorophenyl)-2-oxo-2H-chromen-7-yl)-N-methyl-D-alaninate, 40 [Step 1]: To a stirred solution of ethyl (4-(2-chloro-4-fluorophenyl)-2-oxo-2H-chromen-7-yl)-D-alaninate (Example 26, 300 mg, 0.8 mmol) in DMF (6 mL) was added cesium carbonate (630 mg, 2 mmol) followed by methyl iodide (1.1 mL, 17 mmol) and heated at 80° C. for 24 h. The reaction mixture was diluted with water and extracted with ethyl acetate. The combined organic layer was washed with water and brine, dried over anhydrous Na₂SO₄ and concentrated. The product was purified via combi-flash chromatography to afford ethyl N-(4-(2-chloro-4-fluorophenyl)-2-oxo-2H-chromen-7-yl]-N-methyl-D-alaninate (40, 200 mg). LCMS (ESI) Calcd. for C₂₁H₁₉ClFNO₄: 403, found: [M+H]⁺=404.

Synthesis of N-(4-(2-chloro-4-fluorophenyl)-2-oxo-2H-chromen-7-yl)-N-methyl-D-alanine, 41 [Step 2]: To a stirred solution of ethyl N-(4-(2-chloro-4-fluorophenyl)-2-oxo-2H-chromen-7-yl]-N-methyl-D-alaninate (40, 200 mg, 0.5 mmol) in THF (3 mL) and water (1 mL) was added LiOH·H₂O (83 mg, 2 mmol) at ambient temperature and stirred for 2 h. The mixture was diluted with water, acidified with 10% aqueous citric acid solution and extracted with ethyl acetate. The combined organic layer was washed with brine, dried over anhydrous Na₂SO₄ and concentrated under reduced pressure to afford N-(4-(2-chloro-4-fluorophenyl)-2-oxo-2H-chromen-7-yl)-N-methyl-D-alanine (41, 150 mg). LCMS (ESI) Calcd. for C₁₉H₁₅ClFNO₄: 375, found: [M+H]⁺=376.

Synthesis of (R)-4-(2-chloro-4-fluorophenyl)-7-(methyl(1-oxo-1-(piperidin-1-yl)propan-2-yl)amino)-2H-chromen-2-one, Example 45 [Step 3]: To a stirred solution of N-(4-(2-chloro-4-fluorophenyl)-2-oxo-2H-chromen-7-yl)-N-methyl-D-alanine (41, 110 mg, 0.3 mmol) in DCM (5 mL) was added DIPEA (0.12 mL, 0.9 mmol) followed by piperidine (0.1 mL, 0.1 mmol) and T₃P (50% in EtOAc) (0.3 mL, 0.4 mmol) at 0° C. and stirred at ambient temperature for 4 h. The reaction mixture was concentrated under reduced pressure. The mixture was partitioned between EtOAc and water. The organic layer was washed with brine, dried over Na₂SO₄ and concentrated. The product was purified by reverse phase prep-HPLC and lyophilized to afford (R)-4-(2-chloro-4-fluorophenyl)-7-(methyl(1-oxo-1-(piperidin-1-yl)propan-2-yl)amino)-2H-chromen-2-one (Example 45, 11 mg). LCMS (ESI) Calcd. for C₂₄H₂₄ClFN₂O₃: 442, found: [M+H]⁺=443. ¹H NMR (400 MHz, DMSO-d₆) δ 7.68 (dd, 1H), 7.54 (t, 1H), 7.43-7.39 (m, 1H), 6.84-6.78 (m, 3H), 6.05 (s, 1H), 5.00 (q, 1H), 3.64 (m, 1H), 3.24-3.15 (m, 2H), 2.80 (s, 3H), 1.58-1.44 (m, 4H), 1.43-1.33 (m, 2H), 1.31-1.25 (m, 1H), 1.19 (d, 3H).

Synthesis of (R)-2-((4-(2-chloro-4-fluorophenyl)-2-oxo-2H-chromen-7-yl)(methyl)amino)-N,N-dimethylpropanamide, Example 46 [Step 4]: To a stirred solution of N-(4-(2-chloro-4-fluorophenyl)-2-oxo-2H-chromen-7-yl)-N-methyl-D-alanine (41, 110 mg, 0.3 mmol) in DCM (5 mL) was added DIPEA (0.12 mL, 0.9 mmol) followed by N-methylmethanamine hydrochloride (73 mg, 0.9 mmol) and T₃P, 50% in EtOAc (0.3 mL, 0.4 mmol) at 0° C. and stirred at ambient temperature for 4 h. The reaction mixture was concentrated under reduced pressure, then partitioned between EtOAc and water. The organic layer was washed with brine, dried over Na₂SO₄ and concentrated. This compound was purified by reverse phase prep-HPLC and lyophilized to afford (R)-2-((4-(2-chloro-4-fluorophenyl-2-oxo-2H-chromen-7-yl)(methyl)amino)-N,N-dimethylpropanamide (Example 46, 16 mg). LCMS (ESI) Calcd. for C₂₁H₂₀ClFN₂O₃: 402, found: [M+H]⁺=403. ¹H NMR (400 MHz, DMSO-d₆) δ 7.69 (dd, 1H), 7.54 (dd, 1H), 7.43-7.40 (m, 1H), 6.83-6.78 (m, 3H), 6.05 (s, 1H), 4.98 (q, 1H), 2.86-2.78 (m, 9H), 1.19 (d, 3H).

### Examples 47-48: Synthesis of (R)-4-(2-chloro-4-fluorophenyl)-7-((1-morpholino-1-oxopropan-2-yl)amino)-2H-chromen-2-one, (R)-2-((4-(2-chloro-4-fluorophenyl)-2-oxo-2H-chromen-7-yl)amino)-N-ethylpropanamide

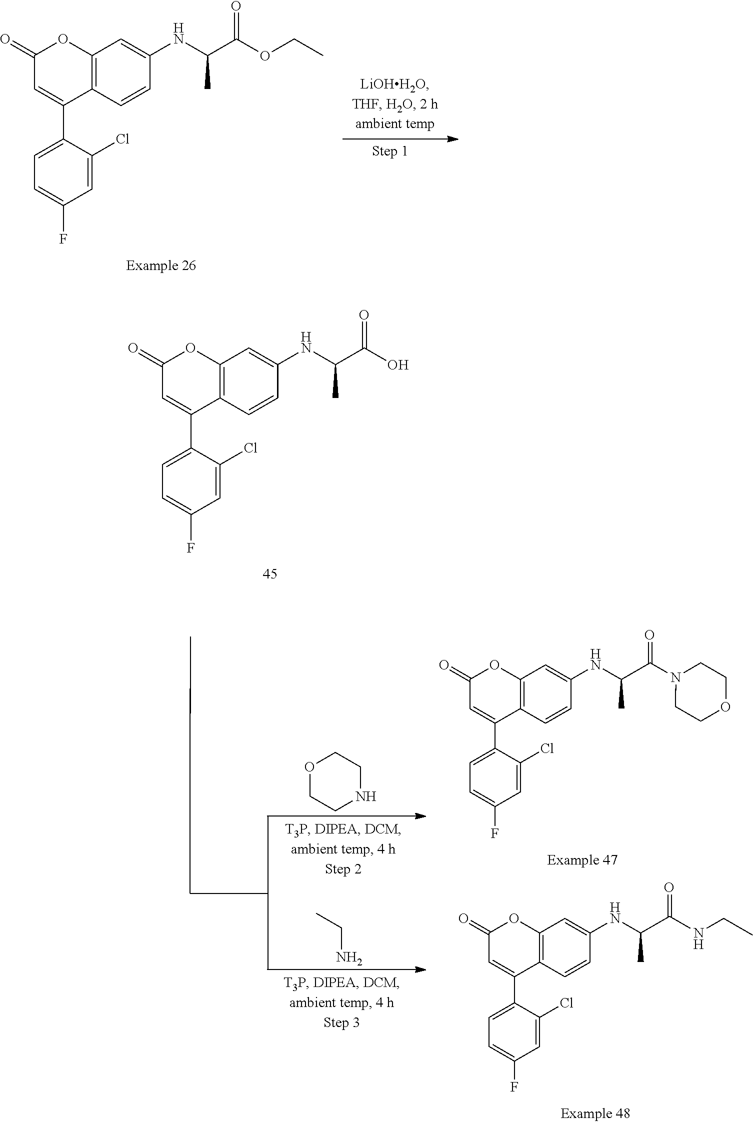

Synthesis of (4-(2-chloro-4-fluorophenyl)-2-oxo-2H-chromen-7-yl)-D-alanine, 45 [Step 1]: To a stirred solution of ethyl (4-(2-chloro-4-fluorophenyl)-2-oxo-2H-chromen-7-yl)-D-alaninate, (Example 26, 365 mg, 1 mmol) in THF (5 mL) and water (1.5 mL) mixture was added LiOH·H₂O (160 mg, 3.7 mmol) and stirred at ambient temperature for 2 h. The reaction mixture was concentrated under reduced pressure. The mixture was diluted with water and acidified with 10% aqueous citric acid solution and extracted with ethyl acetate. The combined organic layer was washed with brine, dried over anhydrous Na₂SO₄ and concentrated under reduced pressure to afford (4-(2-chloro-4-fluorophenyl)-2-oxo-2H-chromen-7-yl)-D-alanine (45, 310 mg). LCMS (ESI): Calcd. for C₁₈H₁₃ClFNO₄: 361, found: [M+H]⁺=362.

Synthesis of (R)-4-(2-chloro-4-fluorophenyl)-7-((1-morpholino-1-oxopropan-2-yl)amino)-2H-chromen-2-one, Example 47 [Step 2]: To a stirred solution of (4-(2-chloro-4-fluorophenyl)-2-oxo-2H-chromen-7-yl)-D-alanine (45, 80 mg, 0.2 mmol) in DCM (5 mL) was added DIPEA (0.1 mL, 0.7 mmol) followed by morpholine (0.1 mL, 0.7 mmol) and T₃P, 50% in EtOAc (0.2 mL, 0.3 mmol) at 0° C. and stirred at ambient temperature for 4 h. The reaction mixture was concentrated under reduced pressure. The mixture was partitioned between EtOAc and water. The organic layer was washed with brine, dried over Na₂SO₄ and concentrated. The compound was purified by reverse phase prep-HPLC and lyophilized to afford (R)-4-(2-chloro-4-fluorophenyl)-7-((1-morpholino-1-oxopropan-2-yl)amino)-2H-chromen-2-one (Example 47, 35 mg). LCMS (ESI) Calcd. for C₂₂H₂₀ClFN₂O₄: 430, found: [M+H]⁺=431. ¹H NMR (400 MHz, DMSO-d₆) δ 7.67-7.65 (dd, 1H), 7.53-7.49 (m, 1H), 7.42-7.37 (m, 1H), 6.92 (d, 1H), 6.70 (d, 1H), 6.62-6.57 (m, 2H), 5.97 (s, 1H), 4.63 (t, 1H), 3.66-3.42 (m, 8H), 1.27 (d, 3H).

Synthesis of (R)-2-((4-(2-chloro-4-fluorophenyl)-2-oxo-2H-chromen-7-yl)amino)-N-ethylpropanamide, Example 48 [Step 3]: To a stirred solution of (4-(2-chloro-4-fluorophenyl)-2-oxo-2H-chromen-7-yl)-D-alanine (45, 140 mg, 0.4 mmol) in DCM (6 mL) were added DIPEA (0.2 mL, 1.2 mmol) followed by ethanamine (0.2 mL, 2.3 mmol). T₃P, 50% in EtOAc (0.3 mL, 0.6 mmol) was added to the solution at 0° C. and stirred at ambient temperature for 4 h. The reaction mixture was concentrated under reduced pressure, then partitioned between EtOAc and water. The organic layer was washed with brine, dried over Na₂SO₄ and concentrated. The product was purified by reverse phase prep-HPLC and lyophilized to afford (R)-2-((4-(2-chloro-4-fluorophenyl)-2-oxo-2H-chromen-7-yl)amino)-N-ethylpropanamide (Example 48, 25 mg). LCMS (ESI) Calcd. for C₂₀H₁₈ClFN₂O₃: 388, found: [M+H]⁺=389. ¹H NMR (400 MHz, DMSO-d₆) δ 8.08 (q, 1H), 7.67 (dd, 1H), 7.52-7.48 (dd, 1H), 7.42 (br s, 1H), 6.97 (d, 1H), 6.70 (d, 1H), 6.56 (d, 1H), 6.44 (s, 1H), 5.98 (s, 1H), 3.92 (t, 1H), 3.08 (br s, 2H), 1.31 (d, 3H), 1.06-0.96 (m, 3H).

### Example 49: Synthesis of 7-(3-oxo-3-(piperidin-1-yl)propyl)-4-(o-tolyl)-2H-chromen-2-one

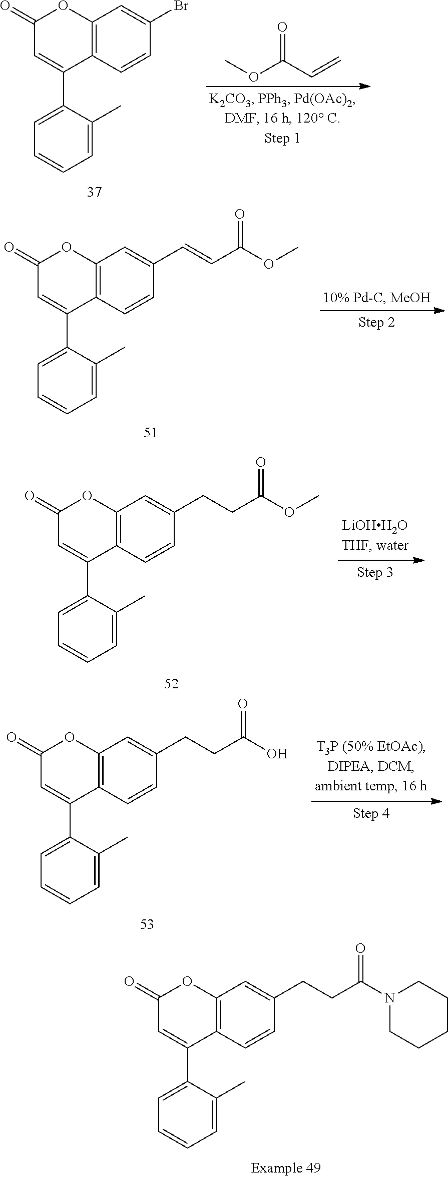

Synthesis of methyl (E)-3-(2-oxo-4-(o-tolyl)-2H-chromen-7-yl)acrylate, 51 [Step 1]: To a stirred solution of 7-bromo-4-(o-tolyl)-2H-chromen-2-one (50, 100 mg, 0.3 mmol) in DMF (3 mL) was added methyl prop-2-enoate (0.14 mL, 1.6 mmol) and the reaction mixture was purged with argon gas for 5 min. Pd(OAc)₂ (7.0 mg, 0.03 mmol) followed by PPh₃ (17 mg, 0.06 mmol) and K₂CO₃ (88 mg, 0.6 mmol) were added to it and heated at 110° C. for 16 h. The reaction mixture was filtered through a celite bed. The filtrate was diluted with EtOAc and washed with water and brine, dried over Na₂SO₄ and concentrated. The product was purified by flash chromatography to afford methyl (E)-3-(2-oxo-4-(o-tolyl)-2H-chromen-7-yl)acrylate (51, 70 mg). LCMS (ESI) Calcd. for C₂₀H₁₆O₄: 320, found: [M+H]⁺=321. ¹H NMR (400 MHz, DMSO-d₆) δ 7.90 (s, 1H), 7.75 (d, 1H), 7.64 (d, 1H), 7.45-7.34 (m, 3H), 7.27 (d, 1H), 6.97 (d, 1H), 6.84 (d, 1H), 6.43 (s, 1H), 3.74 (s, 3H), 2.12 (s, 3H).

Synthesis of methyl 3-(2-oxo-4-(o-tolyl)-2H-chromen-7-yl)propanoate, 52 [Step 2]: A stirred solution of methyl (E)-3-(2-oxo-4-(o-tolyl)-2H-chromen-7-yl)acrylate (51, 130 mg, 0.4 mmol) in ethanol (4 mL) was purged with argon gas for 5 min. 10% Pd—C (10 mg) was added to it and hydrogenated under hydrogen balloon pressure at ambient temperature for 16 h. The reaction mixture was filtered through a celite bed. The filtrate was concentrated and purified by flash chromatography over silica gel to afford methyl 3-(2-oxo-4-(o-tolyl)-2H-chromen-7-yl)propanoate (52, 100 mg). LCMS (ESI) Calcd. for C₂₀H₁₈O₄: 322., found: [M+H]⁺=323. ¹H NMR (400 MHz, DMSO-d₆) δ 7.43-7.35 (m, 4H), 7.25 (d, 1H), 7.17 (d, 1H), 6.89 (d, 1H), 6.32 (s, 1H), 3.58 (s, 3H), 2.95 (t, 2H), 2.70 (t, 2H), 2.11 (s, 3H).

Synthesis of 3-(2-oxo-4-(o-tolyl)-2H-chromen-7-yl)propanoic acid, 53 [Step 3]: To a stirred solution of methyl 3-(2-oxo-4-(o-tolyl)-2H-chromen-7-yl)propanoate (52, 130 mg, 0.4 mmol) in THF (4 mL) and water (1 mL) mixture was added LiOH·H₂O (50 mg, 1.2 mmol) at ambient temperature and stirred for 16 h. The reaction mixture was concentrated under reduced pressure and diluted with water and washed with EtOAc. The aqueous phase was acidified with 10% citric acid solution (pH: 5) and extracted with EtOAc, dried over Na₂SO₄ and concentrated to afford 3-(2-oxo-4-(o-tolyl)-2H-chromen-7-yl)propanoic acid (53, 120 mg). LCMS (ESI) Calcd. for C₁₉H₁₆O₄: 308, found: [M+H]⁺=309.

Synthesis of 7-(3-oxo-3-(piperidin-1-yl)propyl)-4-(o-tolyl)-2H-chromen-2-one, Example 49 [Step 4]: To a stirred solution of 3-(2-oxo-4-(o-tolyl)-2H-chromen-7-yl)propanoic acid (53, 120 mg, 0.4 mmol) in DCM (3 mL) was added piperidine (0.08 mL, 0.8 mmol) followed by DIPEA (0.2 mL, 1.2 mmol) and T₃P, 50% in EtOAc (0.2 mL, 0.6 mmol) at 0° C. and stirred at ambient temperature for 16 h. The reaction mixture was diluted with DCM and washed with water and brine, dried over Na₂SO₄ and the product was purified by reverse phase prep-HPLC and lyophilized to afford 7-(3-oxo-3-(piperidin-1-yl)propyl)-4-(o-tolyl)-2H-chromen-2-one (Example 49, 30 mg). LCMS (ESI) Calcd. for C₂₄H₂₅NO₃: 375, found [M+H]⁺=376. ¹H NMR (400 MHz, DMSO-d₆) δ 7.45-7.40 (m, 4H), 7.37 (d, 1H), 7.25 (d, 1H), 6.88 (d, 1H), 6.31 (s, 1H), 3.41-3.35 (m, 4H), 2.90 (t, 2H), 2.66 (t, 2H), 2.10 (s, 3H), 1.54-1.51 (m, 2H), 1.40-1.38 (m, 4H).

### Examples 51-52: Synthesis of ethyl 2-methyl-3-[4-(o-tolyl)-2-oxo-chromen-7-yl]propanoate, 2-methyl-3-[4-(o-tolyl)-2-oxo-chromen-7-yl]propanoic acid

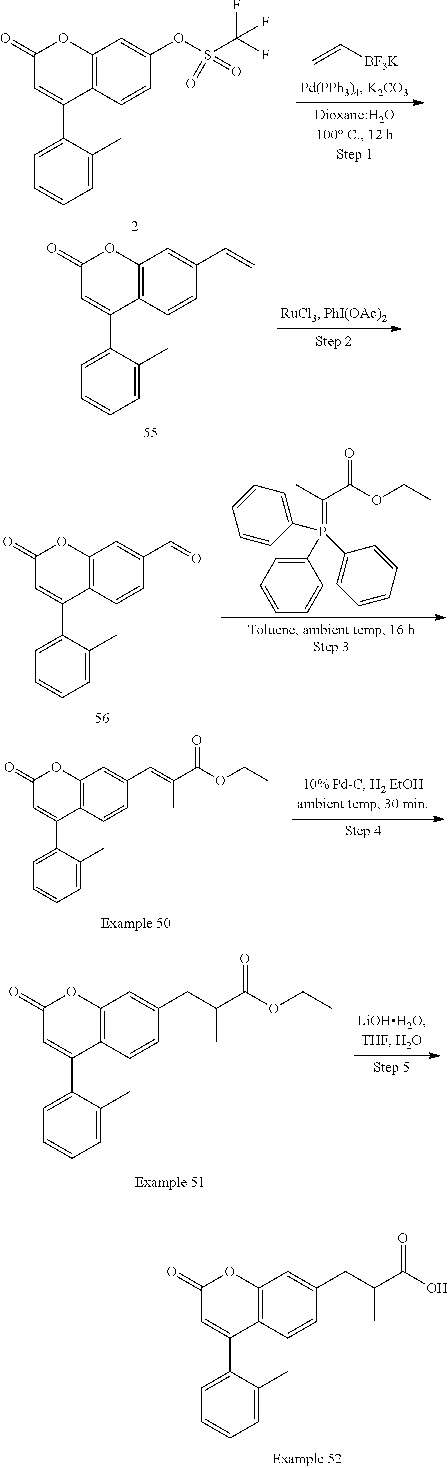

Synthesis of 4-(o-tolyl)-7-vinyl-2H-chromen-2-one, 55 [Step 1]: To a stirred solution 2-oxo-4-(o-tolyl)-2H-chromen-7-yl trifluoromethanesulfonate (2, 3 g, 7.8 mmol) in a mixture of dioxane (24 mL) and water (6 mL) was added potassium vinyltrifluoroborate (1.3 g, 10 mmol) followed by K₂CO₃ (3.2 g, 23 mmol) and degassed with argon for 10 min. Pd(PPh₃)₄ (0.9 g, 0.78 mmol) was added to the solution and heated at 100° C. for 12 h. The reaction mixture was filtered through a celite bed. The filtrate was diluted with EtOAc and washed with water and brine, dried over Na₂SO₄ and concentrated under reduced pressure. The product was purified by flash chromatography to afford 4-(o-tolyl)-7-vinyl-2H-chromen-2-one (55, 1.6 g). LCMS (ESI) Calcd. for C₁₈H₁₄O₂: 262, found: [M+H]⁺=263. ¹H NMR (400 MHz, DMSO-d₆) δ 7.6 (s, 1H), 7.46-7.34 (m, 4H), 7.27 (d, 1H), 6.94 (d, 1H), 6.86-6.79 (m, 1H), 6.35 (s, 1H), 6.07 (d, 1H), 5.47 (d, 1H), 2.12 (s, 3H).

Synthesis of 2-oxo-4-(o-tolyl)-2H-chromene-7-carbaldehyde, 56 [Step 2]: To a stirred solution of 4-(o-tolyl)-7-vinyl-2H-chromen-2-one (55, 700 mg, 2.6 mmol) in DCM (10 mL) and H₂O (2 mL) was added RuCl₃ (55 mg, 0.27 mmol) followed by PhI(OAc)₂ (2.5 g, 8 mmol) and stirred at ambient temperature for 1 h. The reaction mixture was filtered through celite bed. The filtrate was diluted with DCM and washed with water, brine, dried over Na₂SO₄ and concentrated under reduced pressure. The product was purified by combiflash column chromatography to afford 2-oxo-4-(o-tolyl)-2H-chromene-7-carbaldehyde (56, 450 mg). LCMS (ESI): Calcd. for C₁₇H₁₂O₃: 264, found: [M+H]⁺=265. ¹H NMR (400 MHz, DMSO-d₆) δ 10.09 (s, 1H), 7.98 (s, 1H), 7.78 (d, 1H), 7.48-7.36 (m, 3H), 7.3 (d, 1H), 7.18 (d, 1H), 6.57 (s, 1H), 2.1 (s, 3H).

Synthesis of ethyl (E)-2-methyl-3-(2-oxo-4-(o-tolyl)-2H-chromen-7-yl)acrylate, Example 50 [Step 3]: To a stirred solution of 4-(o-tolyl)-2-oxo-chromene-7-carbaldehyde (56, 250 mg, 0.9 mmol) in toluene (8 mL) was added ethyl 2-(triphenyl-15-phosphanylidene)propanoate (686 mg, 1.9 mmol) and stirred at ambient temperature for 5 h. The reaction mixture was concentrated under reduced pressure. The reaction mixture was partitioned between EtOAc and water. Organic layer was washed with brine, dried over Na₂SO₄, and concentrated under reduced pressure. The product was purified by flash column chromatography and lyophilized to afford ethyl (E)-2-methyl-3-(2-oxo-4-(( ) tolyl)-2H-chromen-7-yl)acrylate (Example 50, 270 mg). ¹H NMR (400 MHz, DMSO-d₆) δ 7.64 (d, 2H), 7.47-7.35 (m, 4H), 7.29 (d, 1H), 7.02 (d, 1H), 6.43 (s, 1H), 4.24-4.19 (q, 2H), 2.13 (s, 3H), 2.08 (s, 3H), 1.29 (t, 3H). LCMS (ESI) Calcd. for C₂₂H₂₀O₄: 348, found: [M+H]⁺=349.

Synthesis of ethyl 2-methyl-3-[4-(o-tolyl)-2-oxo-chromen-7-yl]propanoate, Example 51 [Step 4]: To a stirred solution of ethyl (E)-3-[4-(2-chloro-4-fluoro-phenyl)-2-oxo-chromen-7-yl]-2-methyl-prop-2-enoate (Example 50, 100 mg, 0.3 mmol) in EtOH (3 mL) was added 10% Pd—C (10 mg) and hydrogenated under hydrogen balloon pressure at ambient temperature for 1 h. The reaction mixture was filtered through celite bed and purified via prep-HPLC and lyophilized to afford ethyl 2-methyl-3-[4-(o-tolyl)-2-oxo-chromen-7-yl]propanoate (Example 51, 85 mg). ¹H NMR (400 MHz, DMSO-d₆) δ 7.45-7.39 (m, 2H), 7.37-7.34 (m, 2H), 7.26 (d, 1H), 7.13-7.10 (m, 1H), 6.89 (d, 1H), 6.32 (s, 1H), 4.03-3.98 (q, 2H), 3.00-2.93 (m, 1H), 2.83 (m, 2H), 2.10 (s, 3H), 1.10 (t, 6H). LCMS (ESI) Calcd. for C₂₂H₂₂O₄: 350, found: [M+H]⁺=351.

Synthesis of 2-methyl-3-[4-(o-tolyl)-2-oxo-chromen-7-yl]propanoic acid, Example 52 [Step 5]: To a stirred solution of ethyl 2-methyl-3-[4-(o-tolyl)-2-oxo-chromen-7-yl]propanoate (Example 51, 1100 mg, 3.1 mmol) in THF (13 mL) and water (3 mL) was added LiOH. H₂O (396 mg, 9.4 mmol) and stirred at ambient temperature for 4 h. The reaction mixture was concentrated under reduced pressure and partitioned between EtOAc and water. The aqueous layer was acidified with 10% aqueous citric acid solution and extracted with EtOAc. The combined organic layer was washed with brine, dried over Na₂SO₄, and concentrated under reduced pressure. The product was purified by flash column chromatography and lyophilized to afford 2-methyl-3-[4-(o-tolyl)-2-oxo-chromen-7-yl]propanoic acid (Example 52, 920 mg). ¹H NMR (400 MHz, DMSO-d₆) δ 12.21 (s, 1H), 7.43-7.39 (m, 2H), 7.34 (m, 2H), 7.26 (d, 1H), 7.14 (d, 1H), 6.89 (d, 1H), 6.32 (s, 1H), 2.99 (m, 1H), 2.76 (m, 2H), 2.11 (s, 3H), 1.07 (d, 3H). LCMS (ESI) Calcd. for C₂₀H₁₈O₄: 322, found: [M+H]⁺=323.

### Examples 53-54: Synthesis of ethyl (E)-3-(2-oxo-4-(o-tolyl)-2H-chromen-7-yl)acrylate, ethyl 3-(2-oxo-4-(o-tolyl)-2H-chromen-7-yl)propanoate

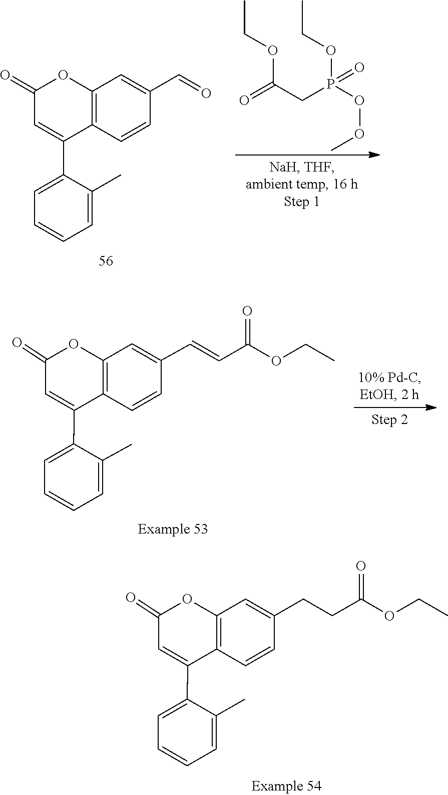

Synthesis of ethyl (E)-3-(2-oxo-4-(o-tolyl)-2H-chromen-7-yl)acrylate, Example 53 [Step 1]: To a stirred solution of 2-oxo-4-(o-tolyl)-2H-chromene-7-carbaldehyde (56, 200 mg, 0.8 mmol) in THF (2 mL), sodium hydride (60% in oil suspension) (40 mg, 1.0 mmol) was added at 0° C. under nitrogen atmosphere and stirred for 15 min. Then ethyl 2-(diethoxyphosphoryl)acetate (0.2 mL, 1.0 mmol) was added to it and continued stirring for 16 h. TLC showed a new spot in nonpolar region and starting was consumed. LCMS also confirmed the formation of product. The reaction mixture was diluted with ethyl acetate and washed with water and brine, dried over Na₂SO₄, and concentrated under reduced pressure to afford 200 mg product. From this 100 mg product was purified by reverse phase prep-HPLC and lyophilized to afford ethyl (E)-3-(2-oxo-4-(o-tolyl)-2H-chromen-7-yl)acrylate (Example 53, 35 mg). LCMS (ESI) Calcd. for C₂₁H₁₈O₄: 334, found: [M+H]⁺=335. ¹H NMR (400 MHz, DMSO-d₆) δ 7.90 (d, 1H), 7.72 (d, 1H), 7.65 (dd, 1H), 7.50-7.33 (m, 3H), 7.27 (d, 1H), 6.97 (d, 1H), 6.82 (d, 1H), 6.43 (s, 1H), 4.21 (q, 2H), 2.12 (s, 3H), 1.27 (t, 3H).

Synthesis of ethyl 3-(2-oxo-4-(o-tolyl)-2H-chromen-7-yl)propanoate, Example 54 [Step 2]: To a stirred solution of ethyl (E)-3-(2-oxo-4-(o-tolyl)-2H-chromen-7-yl)acrylate (Example 53, 100 mg, 0.3 mmol) in ethanol (2 mL), nitrogen was bubbled for 5 min. Then palladium on carbon (160 mg, 0.2 mmol) was added and the reaction was stirred under hydrogen atmosphere for 2 h. TLC showed that starting was consumed and a new spot was formed in polar region. LCMS also confirmed the formation of product. The reaction mixture was filtered through sintered funnel, washed with ethanol and concentrated under reduced pressure. The product was purified by reverse phase prep-HPLC and lyophilized to afford ethyl 3-(2-oxo-4-(o-tolyl)-2H-chromen-7-yl)propanoate (Example 54, 23 mg). LCMS (ESI) Calcd. for: C₂₁H₂O₄: 336, found: [M+H]⁺=337. ¹H NMR (400 MHz, DMSO-d₆) δ 7.48-7.31 (m, 4H), 7.25 (d, 1H), 7.17 (d, 1H), 6.88 (d, 1H), 6.32 (s, 1H), 4.04 (q, 2H), 2.94 (t, 2H), 2.68 (t, 2H), 2.10 (s, 3H), 1.14 (t, 3H).

### Examples 55-56: Synthesis of (R)-4-(2-chloro-4-fluorophenyl)-7-(methyl(1-morpholino-1-oxopropan-2-yl)amino)-2H-chromen-2-one, (R)-2-((4-(2-chloro-4-fluorophenyl)-2-oxo-2H-chromen-7-yl)(methyl)amino)-N-ethylpropanamide

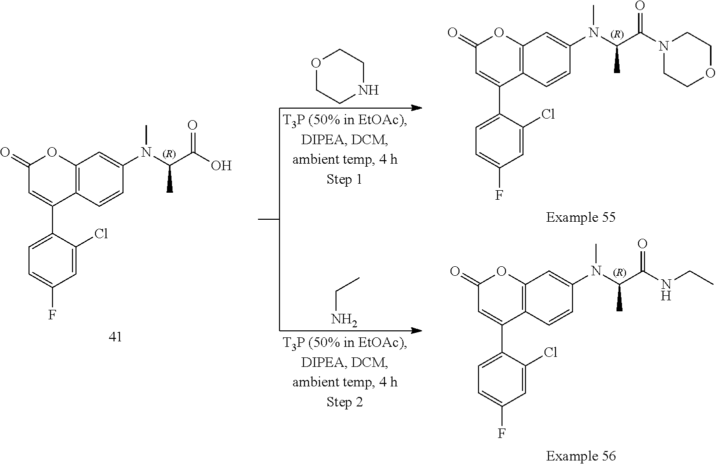

Synthesis of (R)-4-(2-chloro-4-fluorophenyl)-7-(methyl(1-morpholino-1-oxopropan-2-yl)amino)-2H-chromen-2-one, Example 55 [Step 1]: To a stirred solution of N-(4-(2-chloro-4-fluorophenyl)-2-oxo-2H-chromen-7-yl)-N-methyl-D-alanine (41, 240 mg, 0.6 mmol) in DCM (5 mL) was added DIPEA (0.3 mL, 1.9 mmol) followed by morpholine (0.2 mL, 1.9 mmol) and T₃P, 50% in EtOAc (0.6 mL, 1 mmol) at 0° C. followed by stirring at ambient temperature for 4 h. The reaction mixture was concentrated under reduced pressure. The mixture was partitioned between EtOAc and water. The organic phase was washed with brine, dried over Na₂SO₄ and concentrated. The product was purified by prep-HPLC and lyophilized to afford (R)-4-(2-chloro-4-fluorophenyl)-7-(methyl(1-morpholino-1-oxopropan-2-yl)amino)-2H-chromen-2-one (Example 55, 44 mg). LCMS (ESI) Calcd. for C₂₃H₂₂ClFN₂O₄: 444, found: [M+H]⁺=445. ¹H NMR (400 MHz, DMSO-d₆) δ 7.67 (d, 1H), 7.52 (dd, 1H), 7.43-7.39 (m, 1H), 6.85-6.75 (m, 3H), 6.06 (s, 1H), 4.94 (q, 1H), 3.53-3.43 (m, 6H), 3.29-3.25 (m, 2H), 2.81 (s, 3H), 1.20 (d, 3H).

Synthesis of (R)-2-((4-(2-chloro-4-fluorophenyl)-2-oxo-2H-chromen-7-yl)(methyl)amino)-N-ethylpropanamide, Example 56 [Step 2]: To a stirred solution N-(4-(2-chloro-4-fluorophenyl)-2-oxo-2H-chromen-7-yl)-N-methyl-D-alanine (41, 240 mg, 0.6 mmol) in DCM (5 mL) was added DIPEA (0.3 mL, 1.9 mmol) followed by ethanamine (0.3 mL, 3.8 mmol) and T₃P, 50% in EtOAc (0.6 mL, 1 mmol) at 0° C. and stirred at ambient temperature for 4 h. The reaction mixture was concentrated under reduced pressure. The mixture was partitioned between EtOAc and water. The organic phase was washed with brine, dried over Na₂SO₄, and concentrated. The product compound was purified by prep-HPLC and lyophilized to afford (R)-2-((4-(2-chloro-4-fluorophenyl)-2-oxo-2H-chromen-7-yl)(methyl)amino)-N-ethylpropanamide (Example 56, 49 mg). LCMS (ESI) Calcd. for C₂₁H₂₀ClFN₂O₃: 402, found: [M+H]⁺=403. ¹H NMR (400 MHz, DMSO-d₆) δ 7.98 (m, 1H), 7.69 (d, 1H), 7.54 (dd, 1H), 7.44 (m, 1H), 6.79-6.71 (m, 3H), 6.03 (s, 1H), 4.54 (q, 1H), 3.12-3.04 (m, 2H), 2.88 (s, 3H), 1.30 (d, 3H), 1.01 (t, 3H).

### Examples 57-59: Synthesis of methyl (E)-3-(2-oxo-4-(o-tolyl)-2H-chromen-7-yl)but-2-enoate, methyl 3-(2-oxo-4-(o-tolyl)-2H-chromen-7-yl)butanoate, 7-(4-oxo-4-(piperidin-1-yl)butan-2-yl)-4-(o-tolyl)-2H-chromen-2-one

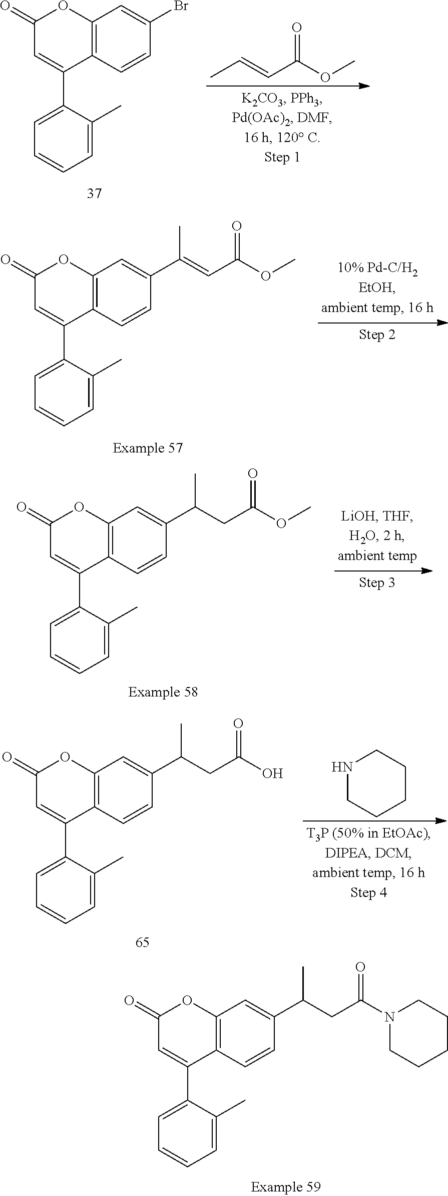

Synthesis of methyl (E)-3-(2-oxo-4-(o-tolyl)-2H-chromen-7-yl)but-2-enoate, Example 57 [Step 1]: To a stirred solution of 7-bromo-4-(o-tolyl)-2H-chromen-2-one (37, 250 mg, 0.8 mmol) in DMF (5 mL) was added methyl (E)-but-2-enoate (0.42 mL, 4.0 mmol) and degassed with argon gas for 5 min. Pd(OAc)₂ (18 mg, 0.08 mmol) followed by PPh₃ (40 mg, 0.2 mmol) and K₂CO₃ (220 mg, 1.6 mmol) were added to it and heated at 110° C. for 16 h. Reaction mixture was filtered through a celite bed and the filtrate liquid was diluted with EtOAc, washed with water and brine, dried over Na₂SO₄. and concentrated under reduced pressure. The product was purified by flash chromatography and via reverse phase prep-HPLC and lyophilized to afford methyl (E)-3-(2-oxo-4-(o-tolyl)-2H-chromen-7-yl)but-2-enoate (Example 57, 40 mg). LCMS (ESI) Calcd. for C₂₁H₁₈O₄: 334, found: [M+H]⁺=335. ¹H NMR (400 MHz, DMSO-d₆) δ 7.71 (d, 1H), 7.51-7.40 (m, 3H), 7.40-7.34 (m, 1H), 7.28 (d, 1H), 6.99 (d, 1H), 6.43 (s, 1H), 6.31 (d, 1H), 3.69 (s, 3H), 2.53 (d, 3H), 2.12 (s, 3H).

Synthesis of methyl 3-(2-oxo-4-(o-tolyl)-2H-chromen-7-yl)butanoate, Example 58 [Step 2]: To a stirred solution of methyl (E)-3-(2-oxo-4-(o-tolyl)-2H-chromen-7-yl)but-2-enoate (Example 57, 60 mg, 0.2 mmol) in ethanol (4 mL) was added 10% Pd—C(10 mg) at ambient temperature and hydrogenated under hydrogen balloon pressure for 16 h. The reaction mixture was filtered through celite bed and the filtrate liquid was concentrated and purified by reverse phase prep-HPLC and lyophilized to afford methyl 3-(2-oxo-4-(o-tolyl)-2H-chromen-7-yl)butanoate (Example 58, 25 mg). LCMS (ESI) Calcd. for C₂₁H₂₀O₄: 336, found: [M+H]⁺=337. ¹H NMR (400 MHz, DMSO-d₆) δ 7.43-7.33 (m, 4H), 7.26 (d, 1H), 7.21 (br s, 1H), 6.90 (d, 1H), 6.32 (s, 1H), 3.54 (s, 3H), 2.69 (d, 2H), 2.11 (s, 3H), 1.24 (d, 3H).

Synthesis of 3-(2-oxo-4-(o-tolyl)-2H-chromen-7-yl)butanoic acid, 65 [Step 3]: To a stirred solution of methyl 3-(2-oxo-4-(o-tolyl)-2H-chromen-7-yl)butanoate (Example 58, 150 mg, 0.5 mmol) in THF (4 mL) and water (1 mL) mixture was added LiOH·H₂O (54 mg, 1.3 mmol) and stirred at ambient temperature for 4 h. The reaction mixture was concentrated under reduced pressure and the product was diluted with water, acidified with 1N HCl and extracted with EtOAc. The organic phase was washed with brine, dried over anhydrous Na₂SO₄. and concentrated to afford 3-(2-oxo-4-(o-tolyl)-2H-chromen-7-yl)butanoic acid (65, 130 mg). LCMS (ESI) Calcd. for C₂₀H₁₈O₄: 322, found: [M+H]⁺=323.

Synthesis of 7-(4-oxo-4-(piperidin-1-yl)butan-2-yl)-4-(o-tolyl)-2H-chromen-2-one, Example 59 [Step 4]: To a solution of 3-(2-oxo-4-(o-tolyl)-2H-chromen-7-yl)butanoic acid (65, 130 mg, 0.4 mmol) and piperidine (0.05 mL, 0.5 mmol) in DCM (5 mL) was added DIPEA (0.16 mL, 1.2 mmol) followed by T₃P, 50% in EtOAc (0.4 mL, 0.6 mmol) at 0° C. and stirred at ambient temperature for 16 h. The reaction mixture was diluted with DCM and washed with water, brine, dried over anhydrous Na₂SO₄. and concentrated and purified via reverse phase prep-HPLC and lyophilized to afford 7-(4-oxo-4-(piperidin-1-yl)butan-2-yl)-4-(o-tolyl)-2H-chromen-2-one (Example 59, 70 mg). LCMS (ESI) Calcd. for C₂₅H₂₇NO₃: 389, found: [M+H]⁺=390. ¹H NMR (400 MHz, DMSO-d₆) δ 7.43-7.33 (m, 4H), 7.25-7.17 (m, 2H), 6.88 (d, 1H), 6.31 (s, 1H), 3.39 (br s, 5H), 2.68-2.61 (m, 2H), 2.11 (s, 3H), 1.52-1.29 (m, 6H), 1.23 (d, 3H).

### Examples 60-61: Synthesis of tert-butyl (2-oxo-4-(o-tolyl)-2H-chromen-7-yl)-L-leucinate, tert-butyl (2-oxo-4-(o-tolyl)-2H-chromen-7-yl)glycinate

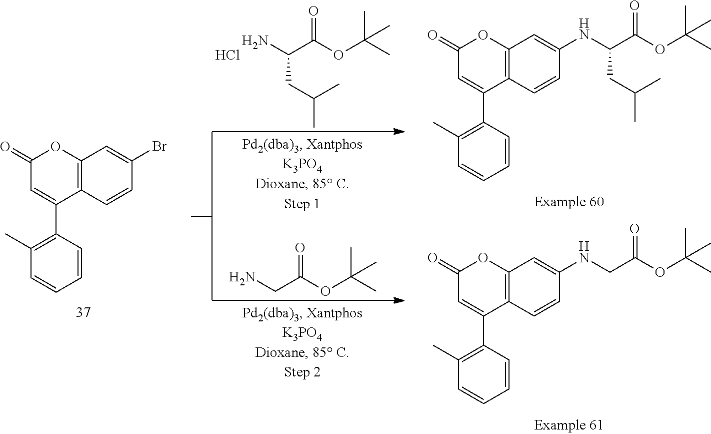

Synthesis of tert-butyl (2-oxo-4-(o-tolyl)-2H-chromen-7-yl)-L-leucinate, Example 60 [Step 1]: An oven dried sealed tube was charged with 7-bromo-4-(o-tolyl)-2H-chromen-2-one (37, 50 mg, 0.2 mmol) followed by tert-butyl-L-leucinate hydrochloride (39 mg, 0.2 mmol) and 1,4-dioxane (2 mL). Potassium phosphate (101 mg, 0.5 mmol) was added, and the mixture was degassed by bubbling Ar for 5 min. XantPhos (18 mg, 31 μmol) and Pd₂(dba)₃ (15 mg, 16 μmol) were added, and the mixture was stirred at 85° C. for 10 h. The reaction mixture was filtered, and partitioned between EtOAc and water. The organic layer was collected and washed with brine, dried over anhydrous Na₂SO₄. and concentrated under reduced pressure. The product was purified by reverse phase prep-HPLC to afford tert-butyl (2-oxo-4-(o-tolyl)-2H-chromen-7-yl)-L-leucinate (Example 60, 11 mg). LCMS (ESI) Calcd. for C₂₆H₃₁NO₄: 421, found: [M+H]⁺=422. ¹H NMR (400 MHz, DMSO-d₆): δ 7.40-7.30 (m, 3H), 7.22-7.19 (d, 1H), 7.02-7.01 (m, 1H), 6.67 (d, 1H), 6.58-6.45 (m, 1H), 6.45 (d, 1H), 5.89 (s, 1H) 3.93 (d, 1H) 2.10 (d, 3H) 1.76-1.71 (m, 1H), 1.63-1.59 (m, 2H), 1.39 (s, 9H), 0.94 (d, 3H), 0.88 (d, 3H).

Synthesis of tert-butyl (2-oxo-4-(o-tolyl)-2H-chromen-7-yl)glycinate, Example 61 [Step 2]: An oven dried sealed tube was charged with 7-bromo-4-(o-tolyl)-2H-chromen-2-one (37, 150 mg, 0.5 mmol) followed by tert-butyl glycinate (0.1 mL, 0.5 mmol), 1,4-dioxane (2 mL). Potassium phosphate (202 mg, 0.9 mmol) was added, and the mixture was degassed by bubbling Ar for 5 min. XantPhos (55 mg, 95 μmol) and Pd₂(dba)₃ (44 mg, 48 μmol) were added, and the mixture was stirred at 85° C. for 10 h. The reaction mixture was filtered and partitioned between EtOAc and water. The organic layer was collected and washed with brine, dried over anhydrous Na₂SO₄. and concentrated under reduced pressure. The product was purified by reverse phase prep-HPLC to afford tert-butyl (2-oxo-4-(o-tolyl)-2H-chromen-7-yl)glycinate (Example 61, 65 mg). LCMS (ESI) Calcd. for C₂₂H₂₃NO₄: 365, found: [M+H]⁺=366. ¹H NMR (400 MHz, DMSO-d₆): δ 7.40-7.30 (m, 3H), 7.22-7.19 (d, 1H), 7.04-7.00 (m, 1H), 6.67 (d, 1H), 6.56-6.46 (m, 1H), 6.47 (d, 1H), 5.89 (s, 1H) 3.91 (d, 2H) 2.11 (s, 3H) 1.43 (s, 9H).

### Examples 62-63: Synthesis of chiral 7-(2-methyl-3-oxo-3-(piperidin-1-yl)propyl)-4-(o-tolyl)-2H-chromen-2-one

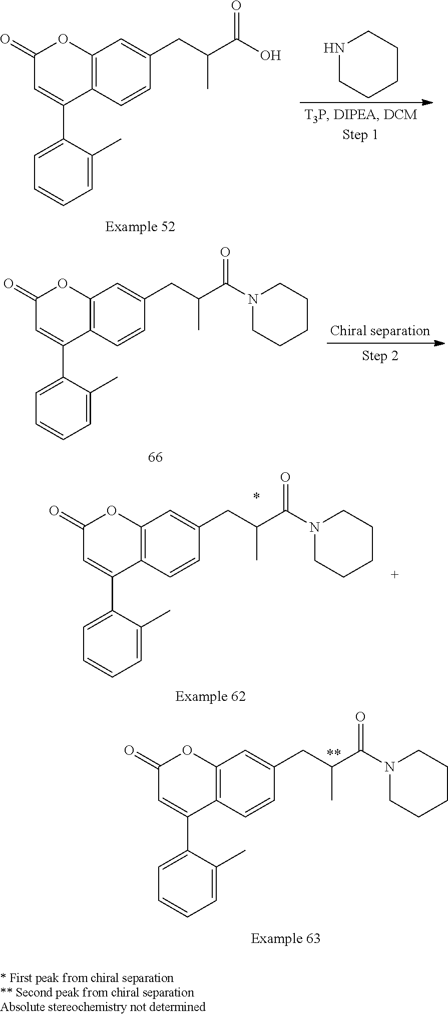 [SMILES: Cc1ccccc1-c1cc(=O)oc2cc(CC(C)C(=O)O)ccc12 ; C1CCNCC1 ; Cc1ccccc1-c1cc(=O)oc2cc(CC(C)C(=O)N3CCCCC3)ccc12 ; Cc1ccccc1-c1cc(=O)oc2cc(CC(C)C(=O)N3CCCCC3)ccc12 ; Cc1ccccc1-c1cc(=O)oc2cc(CC(C)C(=O)N3CCCCC3)ccc12]

Synthesis of 7-(2-methyl-3-oxo-3-(piperidin-1-yl)propyl)-4-(o-tolyl)-2H-chromen-2-one, 65 [Step 1]: To a stirred solution of 2-methyl-3-[4-(o-tolyl)-2-oxo-chromen-7-yl]propanoic acid (Example 52, 200 mg, 0.6 mmol) and piperidine (0.1 mL, 0.9 mmol) in DCM (4 mL) was added DIPEA (0.3 mL, 1.9 mmol) followed by T₃P, 50% in EtOAc (707 mg, 0.9 mmol) at 0° C. and stirred at ambient temperature for 2 h. The reaction mass was diluted with DCM and washed with water and brine, dried over Na₂SO₄. and concentrated under reduced pressure. The product was purified by flash chromatography to afford 7-(2-methyl-3-oxo-3-(piperidin-1-yl)propyl)-4-(o-tolyl)-2H-chromen-2-one (65, 152 mg).

Synthesis of chiral 7-(2-methyl-3-oxo-3-(piperidin-1-yl)propyl)-4-(o-tolyl)-2H-chromen-2-one, Examples 62 and 63 [Step 2]: 7-(2-methyl-3-oxo-3-(piperidin-1-yl)propyl)-4-(o-tolyl)-2H-chromen-2-one (65, 152 mg) was purified by chiral prep-HPLC and lyophilized to afford Peak 1 as 7-(2-methyl-3-oxo-3-(piperidin-1-yl)propyl)-4-(o-tolyl)-2H-chromen-2-one (Example 62, 60 mg) and Peak 2 as 7-(2-methyl-3-oxo-3-(piperidin-1-yl)propyl)-4-(o-tolyl)-2H-chromen-2-one (Example 63, 45 mg). The absolute stereochemistry of these Examples was not determined.

Example 62, 7-(2-methyl-3-oxo-3-(piperidin-1-yl)propyl)-4-(o-tolyl)-2H-chromen-2-one (Peak 1): LCMS (ESI) Calcd. for C₂₅H₂₇NO₃: 389, found: [M+H]⁺=390. ¹H NMR (400 MHz, DMSO-d₆) δ 7.43-7.33 (m, 4H), 7.24 (d, 1H), 7.14 (dd, 1H), 6.87 (d, 1H), 6.31 (s, 1H), 3.50 (s, 1H), 3.38 (s, 1H), 3.28 (s, 1H), 3.22 (q, 2H), 2.95 (q, 1H), 2.70 (q, 1H), 2.09 (s, 3H), 1.47 (d, 2H), 1.35 (s, 2H), 1.18 (d, 1H), 1.02 (d, 4H).

Example 63, 7-(2-methyl-3-oxo-3-(piperidin-1-yl)propyl)-4-(o-tolyl)-2H-chromen-2-one (Peak 2): LCMS (ESI) Calcd. for C₂₅H₂₇NO₃: 389, found: [M+H]⁺=390. ¹H NMR (400 MHz, DMSO-d₆) δ 7.45-7.30 (m, 4H), 7.24 (d, 1H), 7.14 (dd, 1H), 6.87 (d, 1H), 6.31 (s, 1H), 3.53 (s, 1H), 3.37 (s, 1H), 3.28 (s, 1H), 3.22 (q, 2H), 2.95 (q, 1H), 2.70 (q, 1H), 2.09 (s, 3H), 1.47 (d, 2H), 1.36 (s, 2H), 1.18 (d, 1H), 1.02 (d, 4H).

Chiral prep-HPLC method: Chiral separation was performed on an Agilent 1200 series instrument. Column was a Chiralpak IC (250×21 mm), 5μ, operating at ambient temperature and flow rate of 21.0 mL/min. Mobile phase was a mixture of 80% hexane, 10% ethyl acetate and 10% ethanol, held isocratic for up to 25 min. with wavelength of 282 nm.

### Examples 64-65: Synthesis of chiral N,N,2-trimethyl-3-(2-oxo-4-(o-tolyl)-2H-chromen-7-yl)propenamide

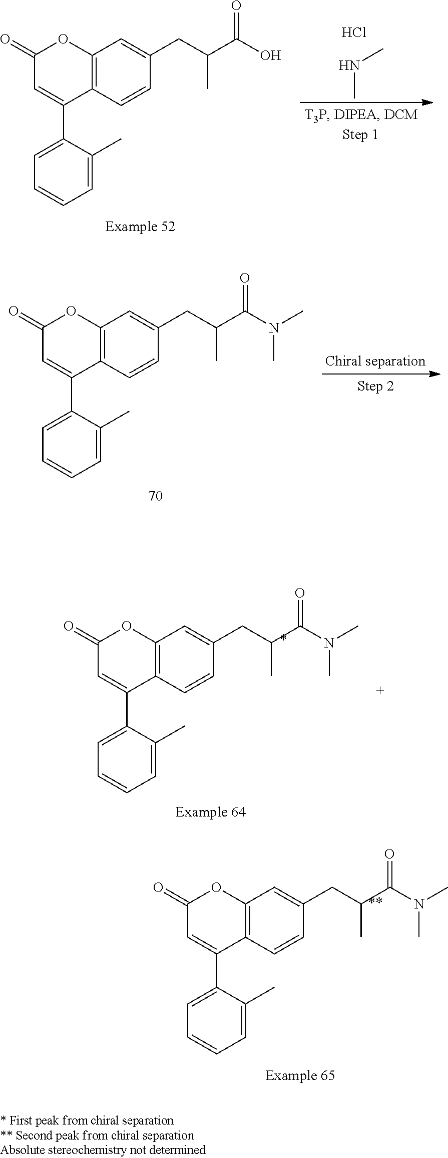

Synthesis of N,N,2-trimethyl-3-(2-oxo-4-(o-tolyl)-2H-chromen-7-yl)propenamide, 70 [Step 1]: To a stirred solution of 2-methyl-3-[4-(o-tolyl)-2-oxo-chromen-7-yl]propanoic acid (Example 52, 200 mg, 0.6 mmol) and (CH₃)₂NH·HCl (95 mg, 1.2 mmol) in DCM (4 mL) was added DIPEA (0.3 mL, 1.9 mmol) followed by T₃P, 50% in EtOAc (707 mg, 0.9 mmol) at 0° C. and stirred at ambient temperature for 2 h. The reaction mass was diluted with DCM and washed with water and brine, dried over Na₂SO₄. and concentrated under reduced pressure. The product was purified by flash chromatography to afford N,N,2-trimethyl-3-(2-oxo-4-(o-tolyl)-2H-chromen-7-yl)propanamide (70, 127 mg).

Synthesis of chiral N,N,2-trimethyl-3-(2-oxo-4-(o-tolyl)-2H-chromen-7-yl)propenamide, Examples 64 and 65 [Step 2]: N,N,2-trimethyl-3-(2-oxo-4-(o-tolyl)-2H-chromen-7-yl)propanamide (70, 127 mg) was purified by chiral prep-HPLC and lyophilized to afford Peak 1 as N,N,2-trimethyl-3-(2-oxo-4-(o-tolyl)-2H-chromen-7-yl)propanamide (Example 64, 36 mg) and Peak 2 as N,N,2-trimethyl-3-(2-oxo-4-(o-tolyl)-2H-chromen-7-yl)propanamide (Example 65, 35 mg). The absolute stereochemistry of these Examples was not determined.

Example 64, N,N,2-trimethyl-3-(2-oxo-4-(o-tolyl)-2H-chromen-7-yl)propanamide (Peak 1): LCMS (ESI) Calcd. for C₂₂H₂₃NO₃: 349, found: [M+H]⁺=350. ¹H NMR (400 MHz, DMSO-d₆) δ 7.43-7.39 (m, 2H), 7.35 (d, 2H), 7.26 (d, 1H), 7.13-7.09 (m, 1H), 6.87 (d, 1H), 6.31 (s, 1H), 3.18 (q, 1H), 2.97-2.91 (m, 4H), 2.76 (s, 3H), 2.67 (q, 1H), 2.10 (s, 3H), 1.01 (d, 3H).

Example 65, N,N,2-trimethyl-3-(2-oxo-4-(o-tolyl)-2H-chromen-7-yl)propanamide (Peak 2): LCMS (ESI) Calcd. for C₂₂H₂₃NO₃: 349, found: [M+H]⁺=350. ¹H NMR (400 MHz, DMSO-d₆) δ 7.43-7.39 (m, 2H), 7.35 (d, 2H), 7.26 (d, 1H), 7.13-7.09 (m, 1H), 6.87 (d, 1H), 6.31 (s, 1H), 3.18 (q, 1H), 2.97-2.91 (m, 4H), 2.76 (s, 3H), 2.67 (q, 1H), 2.10 (s, 3H), 1.01 (d, 3H).

Chiral prep-HPLC method: Chiral separation was performed on an Agilent 1200 series instrument. Column was a Chiralpak IC (250×21 mm), 5μ, operating at ambient temperature and flow rate of 21.0 mL/min. Mobile phase was a mixture of 80% hexane, 10% ethyl acetate and 10% ethanol, held isocratic for up to 25 min. with wavelength of 280 nm.

### Examples 66-67: Synthesis of chiral analogs of N,2-dimethyl-3-(2-oxo-4-(o-tolyl)-2H-chromen-7-yl)propenamide

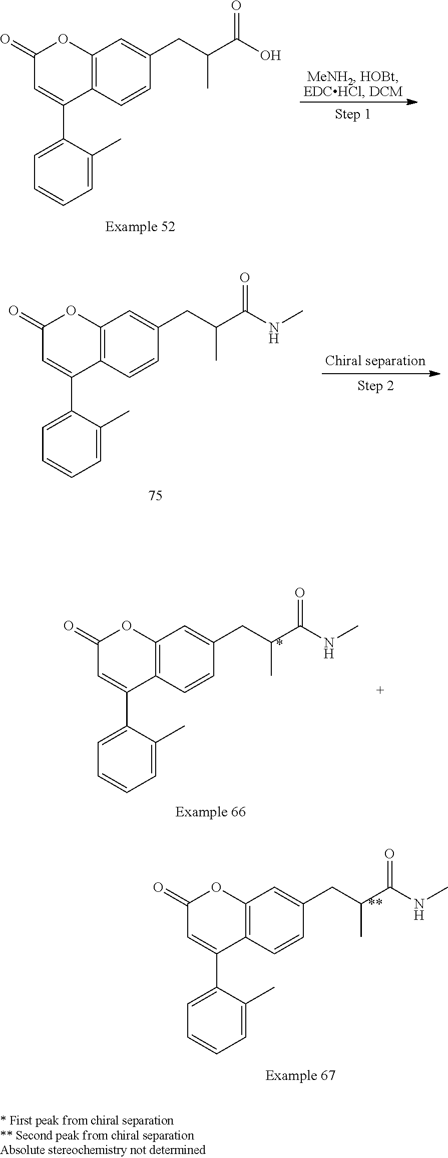 [SMILES: Cc1ccccc1-c1cc(=O)oc2cc(CC(C)C(=O)O)ccc12 ; "MeNH2, HOBt,EDC·HCl, DCM" ; CNC(=O)C(C)Cc1ccc2c(-c3ccccc3C)cc(=O)oc2c1 ; CNC(=O)C(C)Cc1ccc2c(-c3ccccc3C)cc(=O)oc2c1 ; CNC(=O)C(C)Cc1ccc2c(-c3ccccc3C)cc(=O)oc2c1]

Synthesis of N,2-dimethyl-3-(2-oxo-4-(o-tolyl)-2H-chromen-7-yl)propenamide, 75 [Step 1]: To a stirred solution of 2-methyl-3-(2-oxo-4-(o-tolyl)-2H-chromen-7-yl)propanoic acid (Example 52, 200 mg, 0.6 mmol) and CH₃NH₂ HCl (60 mg, 1.9 mmol) in DCM (5 mL) was added HOBt (125 mg, 0.9 mmol) followed by EDC·HCl (180 mg, 0.9 mmol) and DIPEA (0.3 mL, 1.9 mmol) and stirred at ambient temperature for 17 h. The reaction mixture was concentrated under reduced pressure and was partitioned between EtOAc and water. Organic layer was collected and washed with brine, dried over Na₂SO₄ and concentrated. The product was purified by flash column chromatography to afford N,2-dimethyl-3-(2-oxo-4-(o-tolyl)-2H-chromen-7-yl)propanamide (75, 80 mg).

Synthesis of chiral analogs of N,2-dimethyl-3-(2-oxo-4-(o-tolyl)-2H-chromen-7-yl)propenamide, Examples 66 and 67 [Step 2]: N,2-dimethyl-3-(2-oxo-4-(o-tolyl)-2H-chromen-7-yl)propanamide (75, 80 mg) was purified by NP chiral prep-HPLC and lyophilized to afford Peak 1 as N,2-dimethyl-3-(2-oxo-4-(o-tolyl)-2H-chromen-7-yl)propanamide (Example 66, 24 mg) and Peak 2 as N,2-dimethyl-3-(2-oxo-4-(o-tolyl)-2H-chromen-7-yl)propanamide (Example 67, 12 mg). The absolute stereochemistry of these Examples was not determined.

Example 66, Peak 1: N,2-dimethyl-3-(2-oxo-4-(o-tolyl)-2H-chromen-7-yl)propanamide: LCMS (ESI) Calcd. for C₂₁H₂₁NO₃: 335, found: [M+H]⁺=336. ¹H NMR (400 MHz, DMSO-d₆) δ 7.71 (d, 1H), 7.44-7.33 (m, 3H), 7.29 (d, 1H), 7.25 (d, 1H), 7.08 (d, 1H), 6.88 (d, 1H), 6.31 (s, 1H), 2.95 (q, 1H), 2.66 (d, 1H), 2.63 (d, 1H), 2.50 (s, 3H), 2.10 (s, 3H), 1.01 (d, 3H).

Example 67, Peak 2: N,2-dimethyl-3-(2-oxo-4-(o-tolyl)-2H-chromen-7-yl)propanamide: LCMS (ESI) Calcd. for C₂₁H₂₁NO₃: 335, found: [M+H]⁺=336. ¹H NMR (400 MHz, DMSO-d₆) δ 7.71 (d, 1H), 7.45-7.33 (m, 3H), 7.29 (d, 1H), 7.25 (d, 1H), 7.08 (d, 1H), 6.88 (d, 1H), 6.31 (s, 1H), 2.95 (q, 1H), 2.66 (d, 1H), 2.63 (d, 1H), 2.50 (s, 3H), 2.10 (s, 3H), 1.01 (d, 3H).

Chiral prep-HPLC method: Chiral separation was performed on an Agilent 1200 series instrument. Column was a Chiralpak IC (250×21 mm), 5μ, operating at ambient temperature and flow rate of 21.0 mL/min. Mobile phase was a the mixture of 85% hexane, 7.5% ethyl acetate and 7.5% ethanol, held isocratic for up to 30 min. with wavelength of 280 nm.

### Examples 68-69: Synthesis of chiral analogs of 2-methyl-3-(2-oxo-4-(o-tolyl)-2H-chromen-7-yl)propenamide

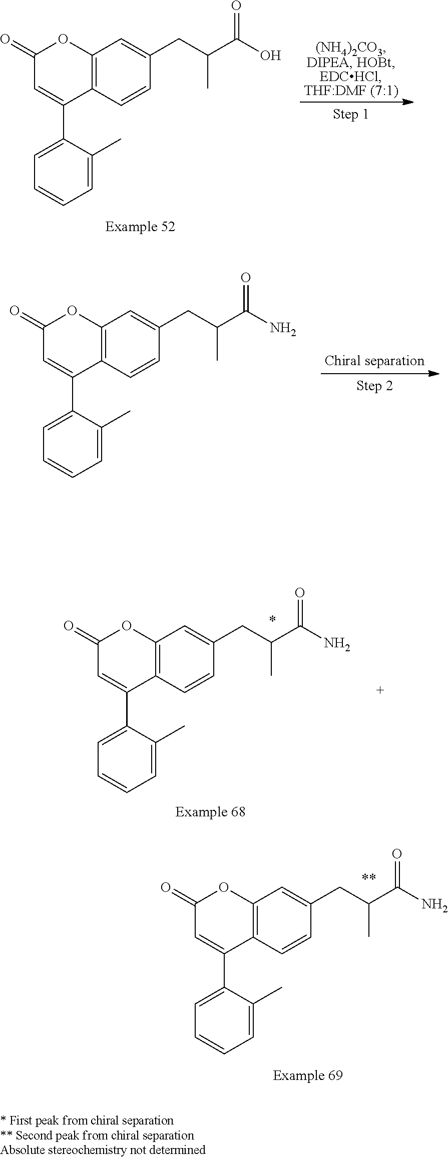 [SMILES: Cc1ccccc1-c1cc(=O)oc2cc(CC(C)C(=O)O)ccc12 ; "(NH4)2CO3,DIPEA, HOBt,EDC·HCl,THF:DMF (7:1)" ; Cc1ccccc1-c1cc(=O)oc2cc(CC(C)C(N)=O)ccc12 ; Cc1ccccc1-c1cc(=O)oc2cc(CC(C)C(N)=O)ccc12 ; Cc1ccccc1-c1cc(=O)oc2cc(CC(C)C(N)=O)ccc12]

Synthesis of 2-methyl-3-(2-oxo-4-(o-tolyl)-2H-chromen-7-yl)propenamide, 80 [Step 1]: To a stirred solution of 2-methyl-3-(2-oxo-4-(o-tolyl)-2H-chromen-7-yl)propanoic acid (Example 52, 150 mg, 0.5 mmol) in THF (4 mL) and DMF (1 mL) was added EDC·HCl (135 mg, 0.7 mmol) followed by HOBt (95 mg, 0.7 mmol), DIPEA (0.3 mL, 1.9 mmol) and (NH₄)₂CO₃ (180 mg, 1.9 mmol) and stirred at ambient temperature for 17 h. The reaction mixture was diluted with water and extracted with EtOAc. The combined organic layer was washed with brine, dried over Na₂SO₄. and concentrated. The product was purified by flash column chromatography to afford 2-methyl-3-(2-oxo-4-(o-tolyl)-2H-chromen-7-yl)propanamide (80, 124 mg).

Synthesis of chiral analogs of 2-methyl-3-(2-oxo-4-(o-tolyl)-2H-chromen-7-yl)propenamide, Examples 68 and 69 [Step 2]: 2-methyl-3-(2-oxo-4-(o-tolyl)-2H-chromen-7-yl)propanamide (80, 124 mg) was purified via NP chiral prep-HPLC and lyophilized to afford Peak 1 as 2-methyl-3-(2-oxo-4-(o-tolyl)-2H-chromen-7-yl)propanamide (Example 68, 43 mg) and Peak 2 as 2-methyl-3-(2-oxo-4-(o-tolyl)-2H-chromen-7-yl)propanamide (Example 69, 34 mg). The absolute stereochemistry of these Examples was not determined. 0279 Example 68, Peak 1:2-methyl-3-(2-oxo-4-(o-tolyl)-2H-chromen-7-yl)propanamide: LCMS (ESI) Calcd. for C₂₀H₁₉NO₃: 321, found: [M+H]⁺=322. ¹H NMR (400 MHz, DMSO-d₆) δ 7.45-7.24 (m, 6H), 7.12 (d, 1H), 6.88 (d, 1H), 6.73 (s, 1H), 6.31 (s, 1H), 2.95 (q, 1H), 2.66 (d, 1H), 2.61 (d, 1H), 2.10 (s, 3H), 1.02 (d, 3H).

Example 69, Peak 2:2-methyl-3-(2-oxo-4-(o-tolyl)-2H-chromen-7-yl)propanamide: LCMS (ESI) Calcd. for C₂₀H₁₉NO₃: 321, found: [M+H]⁺=322. ¹H NMR (400 MHz, DMSO-d₆) δ 7.43-7.24 (m, 6H), 7.12 (d, 1H), 6.88 (d, 1H), 6.72 (s, 1H), 6.31 (s, 1H), 2.94 (q, 1H), 2.66 (d, 1H), 2.61 (d, 1H), 2.11 (s, 3H), 1.02 (d, 3H).

Chiral prep-HPLC method: Chiral separation was performed on Agilent 1200 series instrument. Column was a Chiralpak IC (250×21 mm), 5μ, operating at ambient temperature and flow rate of 21.0 mL/min. Mobile phase was a mixture of 85% hexane, 7.5% ethyl acetate and 7.5% ethanol, held isocratic for up to 30 min. with wavelength of 280 nm.

### Examples 70-71: Synthesis of tert-butyl N-methyl-N-(2-oxo-4-(o-tolyl)-2H-chromen-7-yl)glycinate, Synthesis (2-oxo-4-(o-tolyl)-2H-chromen-7-yl)glycine

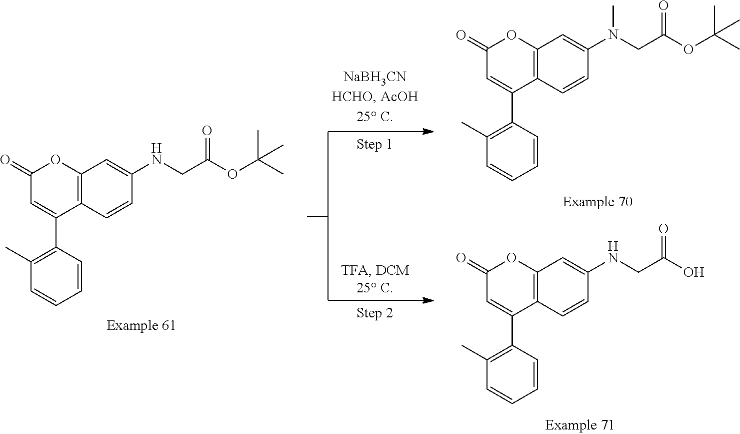 [SMILES: Cc1ccccc1-c1cc(=O)oc2cc(NCC(=O)OC(C)(C)C)ccc12 ; Cc1ccccc1-c1cc(=O)oc2cc(N(C)CC(=O)OC(C)(C)C)ccc12 ; Cc1ccccc1-c1cc(=O)oc2cc(NCC(=O)O)ccc12]

Synthesis of tert-butyl N-methyl-N-(2-oxo-4-(o-tolyl)-2H-chromen-7-yl)glycinate, Example 70 [Step 1]: To an oven dried round bottom flask was added tert-butyl (2-oxo-4-(o-tolyl)-2H-chromen-7-yl)glycinate (Example 61, 50 mg, 0.1 mmol) and acetic acid (0.5 mL). Formaldehyde (0.05 mL, 37% aq. solution, 1.4 mmol) was added dropwise at 25° C. After 15 min., sodium cyanoborohydride (26 mg, 0.4 mmol) was added, and the mixture was stirred for 1 h. at 25° C. Reaction mixture was quenched with water and extracted using ethyl acetate. The combined organic phase was washed successively with saturated aq. NaHCO₃ solution and brine. The organic phase was dried over anhydrous Na₂SO₄, filtered, and concentrated under reduced pressure. The product was purified using reverse phase prep-HPLC to afford N-methyl-N-(2-oxo-4-(o-tolyl)-2H-chromen-7-yl)glycinate (Example 70, 27 mg). LCMS (ESI) Calcd. for C₂₃H₂₅NO₄: 379, found: [M+H]⁺=380. ¹H NMR (400 MHz, DMSO-d₆): δ 7.41-7.31 (m, 3H), 7.22 (d, 1H), 6.73 (d, 1H), 6.63-6.60 (m, 2H), 5.94 (s, 1H), 4.21 (s, 2H) 3.04 (s, 3H) 2.11 (s, 3H) 1.39 (s, 9H).

Synthesis (2-oxo-4-(o-tolyl)-2H-chromen-7-yl)glycine, Example 71 [Step 2]: To a solution of tert-butyl (2-oxo-4-(o-tolyl)-2H-chromen-7-yl)glycinate (Example 61, 100 mg, 0.3 mmol) in DCM (2 mL) was added trifluoroacetic acid (0.2 mL, 2.7 mmol) at 0° C., and the mixture was stirred at 25° C. for 10 h. Reaction mixture was quenched with water, and extracted using 20% MeOH-DCM. The combined organic phase was concentrated under reduced pressure, and the obtained product was purified using reverse phase prep-HPLC and lyophilized to afford (2-oxo-4-(o-tolyl)-2H-chromen-7-yl)glycine (Example 71, 25 mg). LCMS (ESI) Calcd. for C₁₈H₁₅NO₄: 309, found: [M+H]⁺=310. ¹H NMR (400 MHz, DMSO-d₆): δ 7.40-7.30 (m, 3H), 7.19 (d, 1H), 6.63-6.61 (m, 2H), 6.55-6.52 (m, 1H), 6.54 (s, 1H), 5.83 (s, 1H) 3.60 (s, 2H) 3.05 (s, 3H).

### Example 72: Synthesis of 7-((2-oxo-2-(piperidin-1-yl)ethyl)amino)-4-(o-tolyl)-2H-chromen-2-one

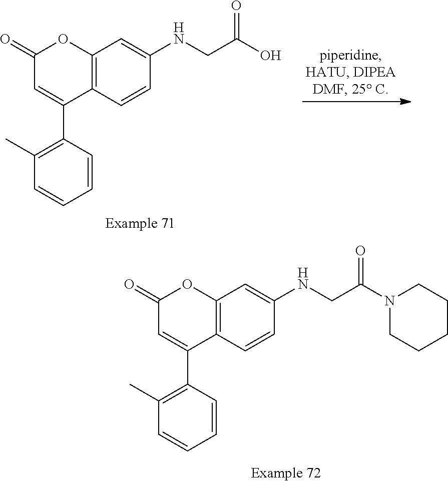 [SMILES: Cc1ccccc1-c1cc(=O)oc2cc(NCC(=O)O)ccc12 ; Cc1ccccc1-c1cc(=O)oc2cc(NCC(=O)N3CCCCC3)ccc12]

Synthesis of 7-((2-oxo-2-(piperidin-1-yl)ethyl)amino)-4-(o-tolyl)-2H-chromen-2-one, Example 72: To a stirring solution of piperidine (35 μL, 0.4 mmol) in DMF (2 mL) was added (2-oxo-4-(o-tolyl)-2H-chromen-7-yl)glycine (Example 71, 100 mg, 0.3 mmol). HATU (135 mg, 0.4 mmol) and DIPEA (65 μL, 0.5 mmol) were added respectively to the reaction mixture at 25° C. After 16 h., the reaction mixture was partitioned between EtOAc and water. The organic phase was washed with brine, dried over anhydrous Na₂SO₄, filtered, and concentrated under reduced pressure. The product was purified by reverse phase prep-HPLC and lyophilized to afford 7-((2-oxo-2-(piperidin-1-yl)ethyl)amino)-4-(o-tolyl)-2H-chromen-2-one (Example 72, 59 mg). LCMS (ESI) Calcd. for C₂₃H₂₄N₂O₃:376, found: [M+H]⁺=377. ¹H NMR (400 MHz, DMSO-d₆): δ 7.42-7.36 (m, 2H), 7.34-7.30 (m, 1H), 7.21-7-19 (m, 1H), 6.70-6.68 (m, 1H), 6.65-6.62 (m, 3H), 5.86 (s, 1H), 4.01-4.00 (m, 2H), 3.47-3.45 (m, 4H), 2.11 (s, 3H), 1.60-1.53 (m, 4H), 1.49-1.39 (m, 2H).

### Example 73: Synthesis of (2-oxo-4-(o-tolyl)-2H-chromen-7-yl)-L-leucine

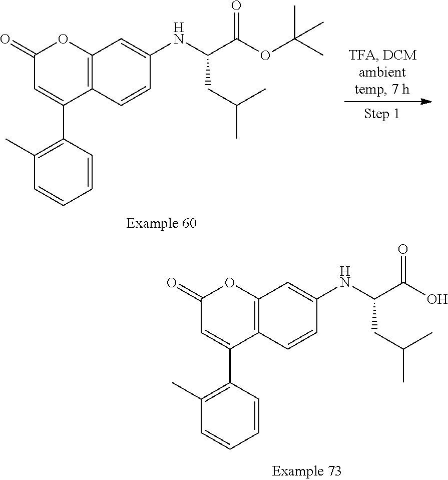 [SMILES: Cc1ccccc1-c1cc(=O)oc2cc(N[C@@H](CC(C)C)C(=O)OC(C)(C)C)ccc12 ; Cc1ccccc1-c1cc(=O)oc2cc(N[C@@H](CC(C)C)C(=O)O)ccc12]

Synthesis of (2-oxo-4-(o-tolyl)-2H-chromen-7-yl)-L-leucine, Example 73 [Step 1]: An oven dried round bottom flask was charged with of tert-butyl (2-oxo-4-(o-tolyl)-2H-chromen-7-yl)-L-leucinate (Example 60, 200 mg, 0.5 mmol) followed by DCM (2 mL). Trifluoroacetic acid (0.4 mL, 4.7 mmol) was added to it and the mixture was stirred at 25° C. for 7 h. The reaction mixture was partitioned between DCM and water. The organic phase was collected and washed with brine, dried over anhydrous Na₂SO₄, filtered, and concentrated under reduced pressure. The product was purified by reverse phase prep-HPLC to afford (2-oxo-4-(o-tolyl)-2H-chromen-7-yl)-L-leucine (Example 73, 37 mg). LCMS (ESI) Calcd. for C₂₂H₂₃NO₄: 365, found: [M+H]⁺=366. ¹H NMR (400 MHz, DMSO-d₆): δ 7.42-7.30 (m, 3H), 7.19 (d, 1H), 6.94-6.92 (m, 1H), 6.63 (d, 1H), 6.56-6.53 (m, 1H), 6.45 (d, 1H), 5.85 (s, 1H), 3.89 (d, 1H), 2.54-2.49 (m, 1H), 2.11 (d, 3H) 1.77-1.71 (m, 1H), 1.63-1.57 (m, 2H), 0.92 (d, 3H), 0.86 (d, 3H).

### Example 74: Synthesis of 7-(1-methoxyethyl)-4-(o-tolyl)-2H-chromen-2-one

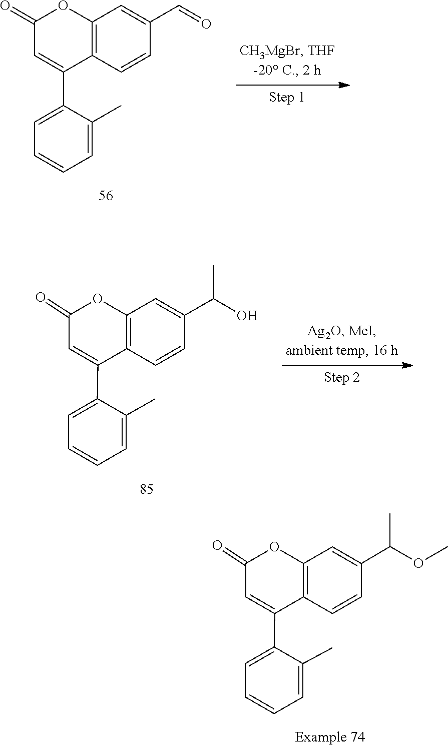 [SMILES: Cc1ccccc1-c1cc(=O)oc2cc(C=O)ccc12 ; Cc1ccccc1-c1cc(=O)oc2cc(C(C)O)ccc12 ; COC(C)c1ccc2c(-c3ccccc3C)cc(=O)oc2c1]

Synthesis of 7-(1-hydroxyethyl)-4-(o-tolyl)-2H-chromen-2-one, 85 [Step 1]: To a stirred solution of 2-oxo-4-(o-tolyl)-2H-chromene-7-carbaldehyde (56, 300 mg, 1.1 mmol) in THF (5 mL) was added methyl magnesium bromide, 3M in diethyl ether (0.3 mL, 0.9 mmol) at −20° C. drop wise under argon atmosphere and stirred for 2 h. The reaction mixture was quenched with saturated aqueous NH₄Cl solution and extracted with EtOAc. Combined organic layer was washed with brine, dried over anhydrous Na₂SO₄ and concentrated. The product was purified by flash column chromatography to afford 7-(1-hydroxyethyl)-4-(o-tolyl)-2H-chromen-2-one (85, 70 mg). LCMS (ESI) Calcd. For C₁₈H₁₆O₃: 280, found: [M+H]⁺=281. ¹H NMR (400 MHz, DMSO-d₆) δ 7.44-7.39 (m, 3H), 7.36 (t, 1H), 7.29-7.25 (m, 2H), 6.94-6.92 (dd, 1H), 6.33 (s, 1H), 5.41 (d, 1H), 4.82-4.80 (m, 1H), 2.11 (s, 3H), 1.35 (d, 3H).

Synthesis of 7-(1-methoxyethyl)-4-(o-tolyl)-2H-chromen-2-one, Example 74 [Step 2]: To a solution of 7-(1-hydroxyethyl)-4-(o-tolyl)-2H-chromen-2-one (85, 70 mg, 0.2 mmol) in methyl iodide (1 ml, 7.5 mmol) was added silver-oxide (115 mg, 0.5 mmol) at 25° C. and stirred for 16 h. The reaction mixture was partitioned between EtOAc and water. The organic layer was washed with brine, dried over NaSO₄, and evaporated under reduced pressure. The product was purified via reverse phase prep-HPLC and lyophilized to afford 7-(1-methoxyethyl)-4-(o-tolyl)-2H-chromen-2-one (Example 74, 20 mg). LCMS (ESI) Calcd. For C₁₉H₁₈O₃: 294, found: [M+H]⁺=295. ¹H NMR (400 MHz, DMSO-d₆) δ 7.44-7.40 (m, 3H), 7.35 (t, 1H), 7.27-7.23 (m, 2H), 6.98 (d, 1H), 6.36 (s, 1H), 4.46-4.42 (m, 1H), 3.17 (s, 3H), 2.13 (s, 3H), 1.34 (d, 3H).

### Example 75: Synthesis of (S)-1-(N-methyl-N-(2-oxo-4-(o-tolyl)-2H-chromen-7-yl)-D-alanyl)piperidine-3-carboxylic acid

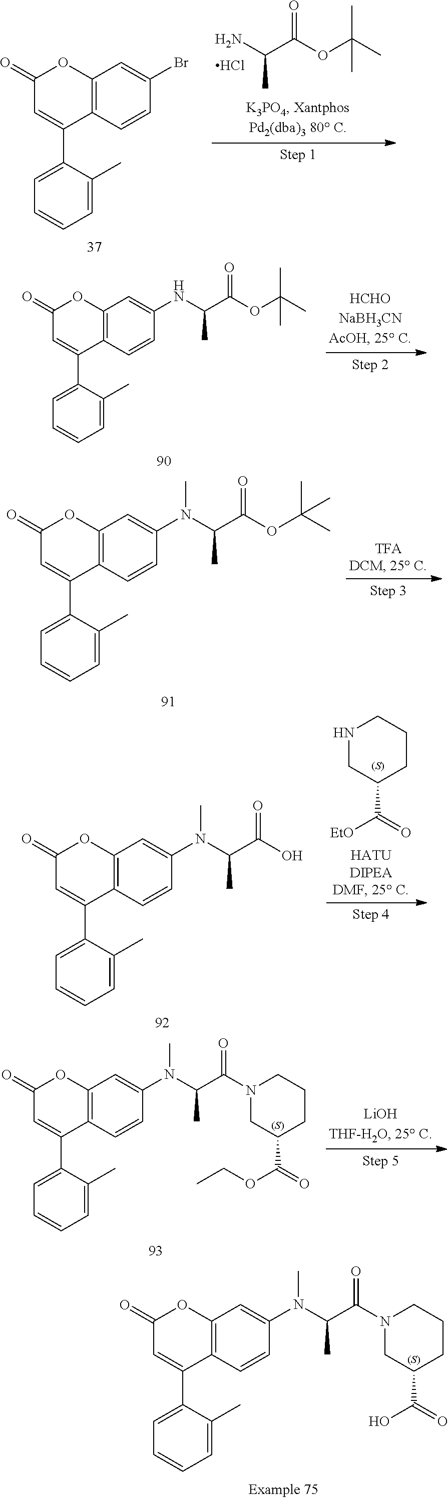

Synthesis of tert-butyl (2-oxo-4-(o-tolyl)-2H-chromen-7-yl)-D-alaninate, 90 [Step 1]: An oven dried sealed tube was charged with of 7-bromo-4-(o-tolyl)-2H-chromen-2-one (37, 1 g, 3.2 mmol) followed by tert-butyl D-alaninate hydrochloride (634 mg, 3.5 mmol), 1,4-dioxane (33 mL). Potassium Phosphate (2 gm, 9.5 mmol) was added to it and degassed with argon for 5 min. by argon bubbling. XantPhos (367 mg, 0.6 mmol) and Pd₂(dba)₃ (290 mg, 0.3 mmol) were added and stirred at 85° C. for 10 h. The reaction mixture was filtered and partitioned between EtOAc and water. Organic layer was collected and washed with brine, dried over anhydrous Na₂SO₄ and concentrated under reduced pressure. The product was purified on silica gel column chromatography to afford tert-butyl (2-oxo-4-(o-tolyl)-2H-chromen-7-yl)-D-alaninate (90, 700 mg). LCMS (ESI) Calcd. for C₂₃H₂₅NO₄: 379, found: [M+H]⁺=380.

Synthesis of tert-butyl-N-methyl-N-(2-oxo-4-(o-tolyl)-2H-chromen-7-yl)-D-alaninate, 91 [Step 2]: An oven dried round bottom flask was charged with of tert-butyl (2-oxo-4-(o-tolyl)-2H-chromen-7-yl)-D-alaninate (90, 300 mg, 0.8 mmol) followed by acetic acid (5 mL), formaldehyde (37% in aq. solution) (0.3 mL, 7.9 mmol). Sodium cyanoborohydride (150 mg, 2.4 mmol) was added to it and at 25° C. for 1 h. The reaction mixture was partitioned between DCM and water. The organic layer was collected and washed with brine, dried over anhydrous Na₂SO₄, and concentrated under reduced pressure. The product N-methyl-N-(2-oxo-4-(o-tolyl)-2H-chromen-7-yl)-D-alaninate (91, 300 mg) was used in the next step. LCMS (ESI) Calcd. for C₂₄H₂₇NO₄: 393, found: [M+H]⁺=394.

Synthesis of N-methyl-N-(2-oxo-4-(o-tolyl)-2H-chromen-7-yl)-D-alanine, 92 [Step 3]: To a solution of N-methyl-N-(2-oxo-4-(o-tolyl)-2H-chromen-7-yl)-D-alaninate (91, 300 mg, 0.8 mmol) in DCM (5 mL) was added trifluoroacetic acid (0.6 mL, 7.6 mmol) at 0° C., and the mixture was stirred at 25° C. for 4 h. The reaction mixture was quenched with water and extracted using 20% MeOH-DCM. The combined organic phase was concentrated under reduced pressure to afford N-methyl-N-(2-oxo-4-(o-tolyl)-2H-chromen-7-yl)-D-alanine (92, 230 mg). LCMS (ESI) Calcd. for C₂₀H₁₉NO₄: 337, found: [M+H]⁺=338.

Synthesis of ethyl (S)-1-(N-methyl-N-(2-oxo-4-(o-tolyl)-2H-chromen-7-yl)-D-alanyl)piperidine-3-carboxylate, 93 [Step 4]: An oven dried round bottom flask was charged with N-methyl-N-(2-oxo-4-(o-tolyl)-2H-chromen-7-yl)-D-alanine (92, 230 mg, 0.7 mmol) followed by ethyl (S)-piperidine-3-carboxylate (0.1 mL, 0.7 mmol) and DMF (3 mL). DIPEA (0.1 mL, 1.0 mmol) and HATU (285 mg, 0.7 mmol) were added to it at 25° C. The reaction mixture was stirred at same temperature for 16 h. The reaction mixture was partitioned between ethyl acetate and water. Organic layer was collected and washed with ice cold brine, dried over anhydrous Na₂SO₄, and concentrated under reduced pressure. The product ethyl (S)-1-(N-methyl-N-(2-oxo-4-(o-tolyl)-2H-chromen-7-yl)-D-alanyl)piperidine-3-carboxylate (93, 300 mg, 48%) was used for next step. LCMS (ESI) Calcd. for C₂₈H₃₂N₂O₅:476, found: [M+H]⁺=477.

Synthesis of (S)-1-(N-methyl-N-(2-oxo-4-(o-tolyl)-2H-chromen-7-yl)-D-alanyl)piperidine-3-carboxylic acid, Example 75 [Step 5]: To a solution of ethyl (S)-1-(N-methyl-N-(2-oxo-4-(o-tolyl)-2H-chromen-7-yl)-D-alanyl)piperidine-3-carboxylate (93, 300 mg, 0.3 mmol) in THF (4.5 mL) and water (0.5 mL) mixture was added lithium hydroxide (40 mg, 1.6 mmol) and the mixture was stirred at 25° C. for 6 h. The reaction mixture was quenched with water and extracted using 20% MeOH-DCM. The combined organic phase was concentrated under reduced pressure. The product was purified using reverse phase prep-HPLC and lyophilized to afford(S)-1-(N-methyl-N-(2-oxo-4-(o-tolyl)-2H-chromen-7-yl)-D-alanyl)piperidine-3-carboxylic acid (Example 75, 86 mg). LCMS (ESI) Calcd. for C₂₆H₂₈N₂O₅: 448, found: [M+H]⁺=449. ¹H NMR (400 MHz, DMSO-d₆ at 100° C.) δ 7.41-7.31 (m, 3H), 7.21 (d, 1H), 6.78 (br s, 3H), 5.91 (s, 1H), 4.95 (d, 1H), 4.27-3.52 (m, 4H), 2.86-2.83 (m, 4H), 2.33 (br s, 1H), 2.15 (s, 3H), 1.93-1.87 (m, 1H), 1.58-1.56 (m, 2H), 1.38 (br s, 1H), 1.26 (d, 3H).

### Example 76: Synthesis N-methyl-N-(2-oxo-4-(o-tolyl)-2H-chromen-7-yl)glycine

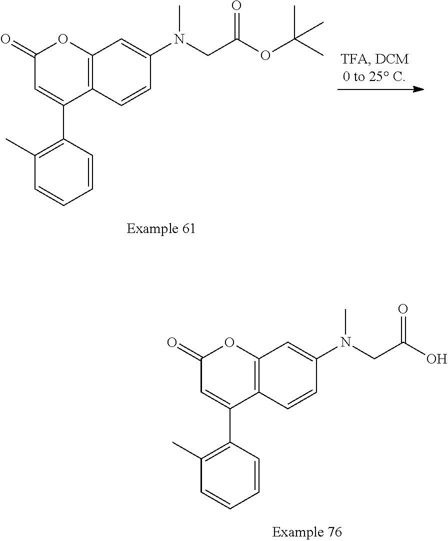 [SMILES: Cc1ccccc1-c1cc(=O)oc2cc(N(C)CC(=O)OC(C)(C)C)ccc12 ; Cc1ccccc1-c1cc(=O)oc2cc(N(C)CC(=O)O)ccc12]

Synthesis N-methyl-N-(2-oxo-4-(o-tolyl)-2H-chromen-7-yl)glycine, Example 76: To a solution of tert-butyl N-methyl-N-(2-oxo-4-(o-tolyl)-2H-chromen-7-yl)glycinate (Example 61, 100 mg, 0.3 mmol) in DCM (1 mL) was added TFA (0.5 mL, 6.5 mmol) 0° C., and the mixture was stirred at 25° C. After 2 h., the reaction mixture was concentrated under reduced pressure, and the product was purified by reverse phase prep-HPLC to afford N-methyl-N-(2-oxo-4-(o-tolyl)-2H-chromen-7-yl)glycine (Example 76, 10 mg). LCMS (ESI) Calcd. for C₁₉H₁₇NO₄: 323, found: [M+H]⁺=324. ¹H NMR (400 MHz, DMSO-d₆): δ 7.41-7.37 (m, 2H), 7.35-7.31 (m, 1H), 7.22-7.20 (m, 1H), 6.73-6.70 (m, 1H), 6.62-6.60 (m, 2H), 5.92 (s, 1H), 4.17 (s, 2H), 3.04 (s, 3H), 2.12 (s, 3H).

### Example 77: Synthesis of N-methyl-N-(1-(2-oxo-4-(o-tolyl)-2H-chromen-7-yl)ethyl)acetamide

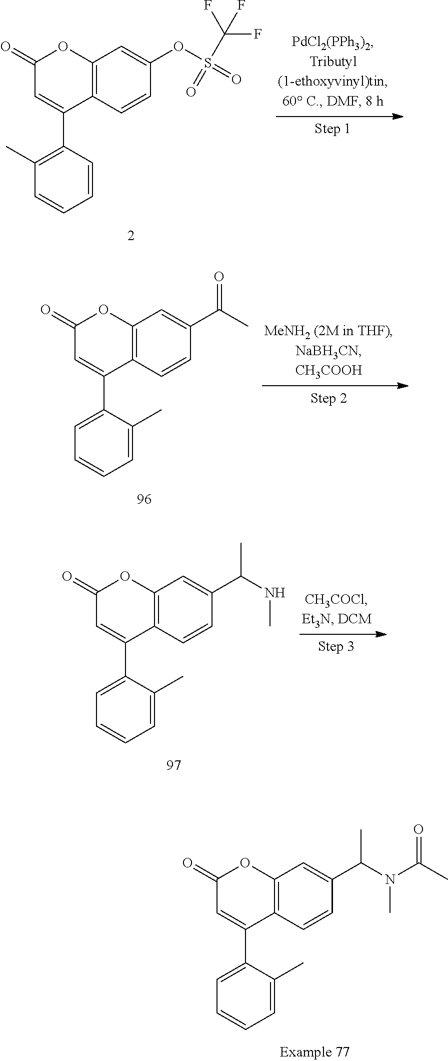 [SMILES: Cc1ccccc1-c1cc(=O)oc2cc(OS(=O)(=O)C(F)(F)F)ccc12 ; CC(=O)c1ccc2c(-c3ccccc3C)cc(=O)oc2c1 ; CNC(C)c1ccc2c(-c3ccccc3C)cc(=O)oc2c1 ; CC(=O)N(C)C(C)c1ccc2c(-c3ccccc3C)cc(=O)oc2c1]

Synthesis of 7-acetyl-4-(o-tolyl)-2H-chromen-2-one, 96 [Step 1]: To a stirred solution of 2-oxo-4-(o-tolyl)-2H-chromen-7-yl trifluoromethanesulfonate (2, 1.5 g, 4.0 mmol) and tributyl (1-ethoxyvinyl) tin (1.5 mL, 4.3 mmol) in DMF (4 mL), bis(triphenylphosphine) palladium (II) chloride (140 mg, 0.2 mmol) was added and the resulting mixture was stirred at ambient temperature for 30 min. The flask was then equipped with a reflux condenser, and the reaction mixture was heated at 60° C. for an additional 8 h. The reaction mixture was diluted with EtOAc and washed with 1N HCl solution. The organic layer was collected and was further washed with brine, dried over Na₂SO₄, and concentrated under reduced pressure to afford the product. The product was purified through flash column chromatography to yield 7-acetyl-4-(o-tolyl)-2H-chromen-2-one (96, 150 mg). ¹H NMR (400 MHz, DMSO-d₆) δ 8.02 (s, 1H), 7.80 (d, 1H), 7.48-7.36 (m, 3H), 7.28 (d, 1H), 6.54 (s, 1H), 2.65 (s, 3H), 2.12 (s, 3H).

Synthesis of 7-(1-(methylamino)ethyl)-4-(o-tolyl)-2H-chromen-2-one, 97 [Step 2]: To a stirred solution of 7-acetyl-4-(o-tolyl)-2H-chromen-2-one (96, 150 mg, 0.5 mmol) in MeNH₂ (2M in THF) (3 ml, 5.4 mmol), acetic acid (0.05 mL, 0.5 mmol) was added and stirred at ambient temperature for 15 min. Then the reaction mixture was cooled and NaBH₃CN (70 mg, 1.1 mmol) was added and the reaction mixture was stirred at ambient temperature for 16 h. The reaction mixture was diluted with EtOAc and washed with NaHCO₃, water and brine. The organic phase was dried over Na₂SO₄, filtered and evaporated to yield 7-(1-(methylamino)ethyl)-4-(o-tolyl)-2H-chromen-2-one (97, 150 mg). LCMS (ESI): Calcd. for C₁₉H₁₉NO₂: 293, found: [M+H]⁺=294.

Synthesis of N-methyl-N-(1-(2-oxo-4-(o-tolyl)-2H-chromen-7-yl)ethyl)acetamide, Example 77 [Step 3]: To a stirred solution of 7-(1-(methylamino)ethyl)-4-(o-tolyl)-2H-chromen-2-one (97, 150 mg, 0.5 mmol) in DCM (3 mL) was added Et₃N (0.2 mL, 1.3 mmol) followed by acetyl chloride (0.1 mL, 0.8 mmol) at 0° C. The reaction mixture was stirred at ambient temperature for 2 h. After completion, the reaction mass was partitioned between DCM and water. The organic layer was washed with ice cold water and brine, dried over Na₂SO₄, and concentrated under reduced pressure. The product was submitted for reverse phase prep-HPLC. The fraction obtained was lyophilized to yield N-methyl-N-(1-(2-oxo-4-(o-tolyl)-2H-chromen-7-yl)ethyl)acetamide (Example 77, 120 mg). LCMS (ESI) Calcd. for C₂₁H₂₁NO₃: 335, found: [M+H]⁺=336. ¹H NMR (400 MHz, DMSO-d₆ at 100° C.) δ 7.46-7.39 (m, 2H), 7.37-7.33 (m, 2H), 7.25 (d, 1H), 7.17 (d, 1H), 6.98 (d, 1H), 6.30 (s, 1H), 5.74 (s, 1H), 2.72 (s, 3H), 2.14 (s, 3H), 2.08 (s, 3H), 1.52 (s, 3H).

### Example 78: Synthesis of 7-(1-(dimethylamino)ethyl)-4-(o-tolyl)-2H-chromen-2-one

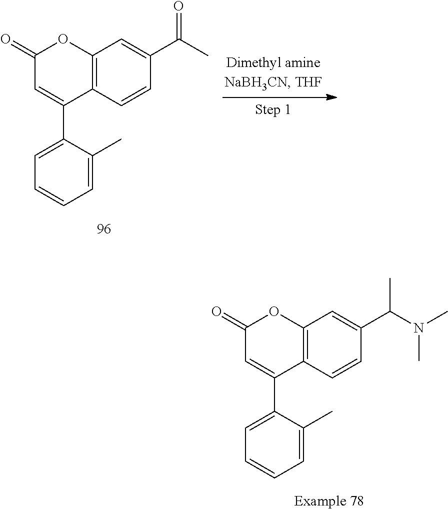 [SMILES: CC(=O)c1ccc2c(-c3ccccc3C)cc(=O)oc2c1 ; Cc1ccccc1-c1cc(=O)oc2cc(C(C)N(C)C)ccc12]

Synthesis of 7-(1-(dimethylamino)ethyl)-4-(o-tolyl)-2H-chromen-2-one, Example 78 [Step 1]: To a stirred solution of 7-acetyl-4-(o-tolyl)-2H-chromen-2-one (96, 100 mg, 0.4 mmol) in dimethylamine (2M in THF) (1.0 mL, 3.6 mmol), acetic acid (0.1 mL) was added and stirred at ambient temperature for 15 min. Then the reaction mixture was cooled and NaBH₃CN (45 mg, 0.7 mmol) was added, and the reaction mixture was stirred at ambient temperature for 48 h. TLC and LCMS showed full consumption of starting material with formation of new polar spot. The reaction mixture was diluted with EtOAc and washed with NaHCO₃, water and brine. The organic phase was dried over Na₂SO₄, filtered, and evaporated to yield the product. The product was purified by reverse phase prep-HPLC and lyophilized to yield 7-(1-(dimethylamino)ethyl)-4-(o-tolyl)-2H-chromen-2-one (Example 78, 60 mg). LCMS (ESI): Calcd. for C₂₀H₂₁NO₂: 307, found: [M+H]⁺=308. ¹H NMR (400 MHz, DMSO-d₆) δ 7.46-7.34 (m, 4H), 7.27-7.23 (m, 2H), 6.93 (d, 1H), 6.33 (s, 1H), 3.38 (q, 1H), 2.12 (s, 3H), 2.11 (s, 6H), 2.27 (d, 3H).

### Example 79: Synthesis of (S)-1-((2-oxo-4-(o-tolyl)-2H-chromen-7-yl)glycyl)piperidine-3-carboxylic acid

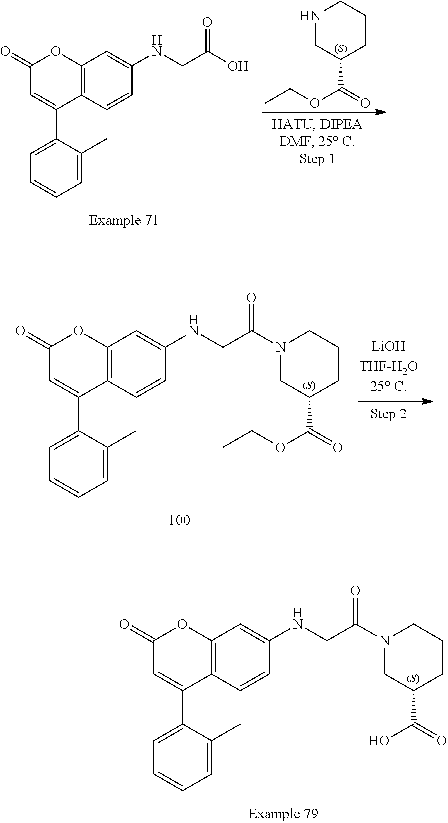

Synthesis of ethyl (S)-1-((2-oxo-4-(o-tolyl)-2H-chromen-7-yl)glycyl)piperidine-3-carboxylate, 100 [Step 1]: An oven dried round bottom flask was charged with (2-oxo-4-(o-tolyl)-2H-chromen-7-yl)glycine (Example 71, 450 mg, 0.9 mmol) followed by ethyl (S)-piperidine-3-carboxylate (0.1 mL, 1.0 mmol) and DMF (6 mL). DIPEA (0.2 mL, 1.4 mmol) and HATU (375 mg, 1.0 mmol) were added to the flask at 25° C. The reaction mixture was stirred at 25° C. for 16 h. The reaction mixture was then partitioned between ethyl acetate and water. The organic layer was collected and washed with ice cold water and brine, dried over anhydrous Na₂SO₄ and concentrated under reduced pressure. The ethyl (S)-1-((2-oxo-4-(o-tolyl)-2H-chromen-7-yl)glycyl)piperidine-3-carboxylate (100, 550 mg) was used for next reaction. LCMS (ESI) Calcd. for C₂₆H₂₈N₂O₅: 448, found: [M+H]⁺=449.

Synthesis of (S)-1-((2-oxo-4-(o-tolyl)-2H-chromen-7-yl)glycyl)piperidine-3-carboxylic acid, Example 79 [Step 2]: To a solution of ethyl (S)-1-((2-oxo-4-(o-tolyl)-2H-chromen-7-yl)glycyl)piperidine-3-carboxylic acid (100, 550 mg, 0.6 mmol) in THF (4.5 mL) and water (0.5 mL) mixture was added lithium hydroxide (70 mg, 2.8 mmol) and the mixture was stirred at 25° C. for 6 h. The reaction mixture was quenched with water and extracted using 20% MeOH-DCM. The combined organic phase was concentrated under reduced pressure. The product was purified using reverse phase prep-HPLC to afford(S)-1-((2-oxo-4-(o-tolyl)-2H-chromen-7-yl)glycyl)piperidine-3-carboxylic acid (Example 79, 100 mg). LCMS (ESI) Calcd. for C₂₄H₂₄N₂O₅: 420, found: [M+H]⁺=421. ¹H NMR (400 MHz, DMSO-d₆): δ 7.41-7.30 (m, 3H), 7.20-7.18 (m, 1H), 6.68-6.44 (m, 4H), 5.83 (s, 1H), 4.06-3.92 (m, 2H), 3.78-3.60 (m, 2H), 3.38-3.17 (m, 2H) 2.38-2.32 (m, 1H), 2.14 (s, 3H), 1.99-1.95 (m, 1H), 1.69-1.47 (m, 2H), 1.27-1.21 (m, 1H).

### Example 80: Synthesis of (S)-1-((2-oxo-4-(o-tolyl)-2H-chromen-7-yl)-D-alanyl)piperidine-3-carboxylic acid

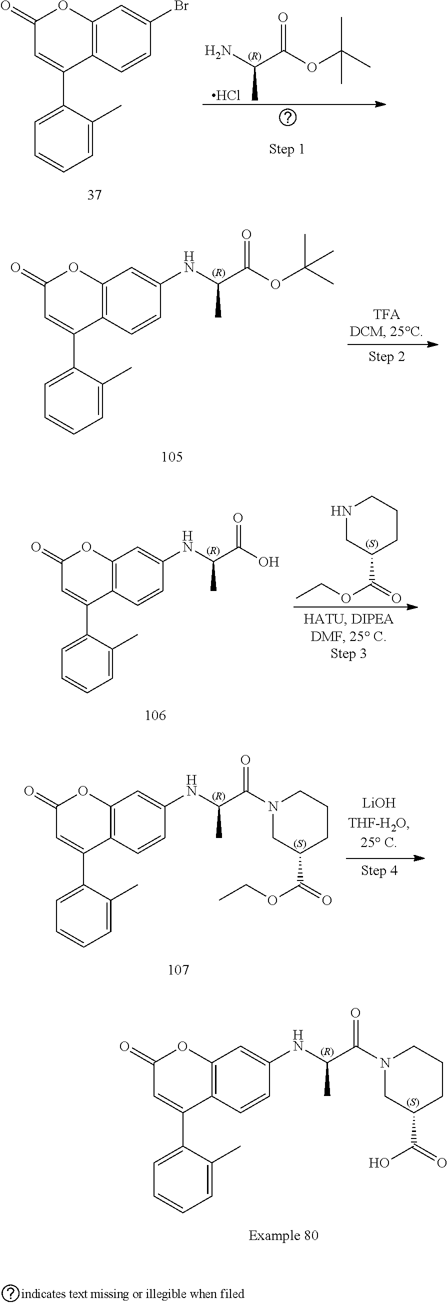

Synthesis of tert-butyl (2-oxo-4-(o-tolyl)-2H-chromen-7-yl)-D-alaninate, 105 [Step 1]: An oven dried sealed tube was charged with 7-bromo-4-(o-tolyl)-2H-chromen-2-one (37, 1.0 g, 3.2 mmol), followed by tert-butyl D-alaninate hydrochloride (635 mg, 3.5 mmol) and 1,4-dioxane (35 mL). Potassium phosphate (2.0 g, 9.5 mmol) was added to it and degassed with argon for 5 min. XantPhos (365 mg, 0.6 mmol) and Pd₂(dba)₃ (290 mg, 0.3 mmol) were added and stirred at 80° C. for 10 h. The reaction mixture was filtered and partitioned between EtOAc and water. The organic layer was collected and washed with brine, dried over anhydrous Na₂SO₄ and concentrated under reduced pressure. The product was purified over silica gel column chromatography to afford tert-butyl (2-oxo-4-(o-tolyl)-2H-chromen-7-yl)-D-alaninate (105, 700 mg). LCMS (ESI) Calcd. for C₂₃H₂₅NO₄: 379, found: [M+H]⁺=380.

Synthesis of (2-oxo-4-(o-tolyl)-2H-chromen-7-yl)-D-alanine, 106 [Step 2]: To a solution of tert-butyl (2-oxo-4-(o-tolyl)-2H-chromen-7-yl)-D-alaninate (105, 250 mg, 0.7 mmol) in DCM (5 mL) was added trifluoroacetic acid (0.5 mL, 6.6 mmol) at 0° C., and the mixture was stirred at 25° C. for 4 h. The reaction mixture was quenched with water and extracted using 20% MeOH-DCM. The combined organic phase was concentrated under reduced pressure to afford (2-oxo-4-(o-tolyl)-2H-chromen-7-yl)-D-alanine (106, 200 mg). LCMS (ESI) Calcd. for C₁₉H₁₇NO₄: 323, found: [M+H]⁺=324.

Synthesis of ethyl (S)-1-((2-oxo-4-(o-tolyl)-2H-chromen-7-yl)-D-alanyl)piperidine-3-carboxylate, 107 [Step 3]: An oven dried round bottom flask was charged with (2-oxo-4-(o-tolyl)-2H-chromen-7-yl)-D-alanine (106, 200 mg, 0.6 mmol) followed by ethyl (S)-piperidine-3-carboxylate (0.1 mL, 0.7 mmol) and DMF (3 mL). DIPEA (0.1 mL, 0.9 mmol) and HATU (260 mg, 0.7 mmol) were added to the flask at 25° C. The reaction mixture was stirred at 25° C. for 16 h. The reaction mixture was then partitioned between ethyl acetate and water. The organic layer was collected and washed with ice cold water and brine, dried over anhydrous Na₂SO₄ and concentrated under reduced pressure. The product ethyl (S)-1-((2-oxo-4-(o-tolyl)-2H-chromen-7-yl)-D-alanyl)piperidine-3-carboxylate (107, 250 mg) was used for next reaction. LCMS (ESI) Calcd. for C₂₇H₃₀N₂O₅: 462, found: [M+H]⁺=463.

Synthesis of (S)-1-((2-oxo-4-(o-tolyl)-2H-chromen-7-yl)-D-alanyl)piperidine-3-carboxylic acid, Example 80 [Step 4]: To a solution of ethyl (S)-1-((2-oxo-4-(o-tolyl)-2H-chromen-7-yl)-D-alanyl)piperidine-3-carboxylate (107, 250 mg, 0.5 mmol) in THF (4.5 mL) and water (0.5 mL) mixture was added lithium hydroxide (65 mg, 2.7 mmol) and the mixture was stirred at 25° C. for 6 h. The reaction mixture was quenched with water and extracted using 20% MeOH-DCM. The combined organic phase was concentrated under reduced pressure. The product was purified using reverse phase prep-HPLC to afford(S)-1-((2-oxo-4-(o-tolyl)-2H-chromen-7-yl)-D-alanyl)piperidine-3-carboxylic acid (Example 80, 31 mg). LCMS (ESI) Calcd. for C₂₅H₂₆N₂O₅: 434, found: [M+H]⁺=435. ¹H NMR (400 MHz, DMSO-d₆): δ 7.40-7.30 (m, 3H), 7.19 (d, 1H), 6.67-6.53 (m, 4H), 5.83 (s, 1H), 4.63 (br s, 1H), 4.06-3.80 (m, 3H), 2.38-2.32 (m, 1H), 2.14 (s, 3H), 1.95-1.78 (m, 2H), 1.69-1.46 (m, 3H), 1.31-1.30 (m, 3H).

### Examples 81-82: Synthesis of (S)-1-(3-(2-oxo-4-(o-tolyl)-2H-chromen-7-yl)butanoyl)piperidine-3-carboxylic acid, (S)-1-(3-(2-oxo-4-(o-tolyl)-2H-chromen-7-yl)butanoyl)piperidine-3-carboxylic acid

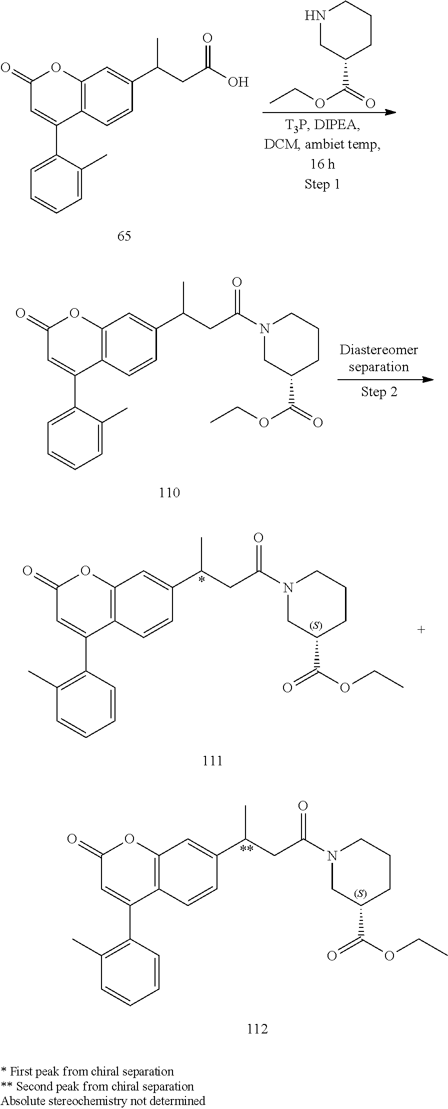

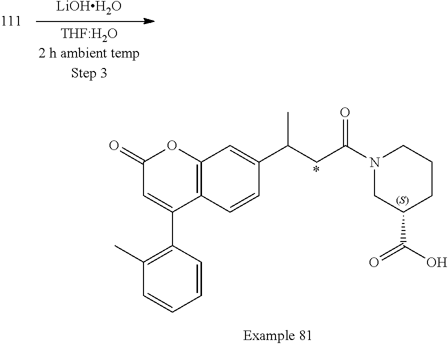

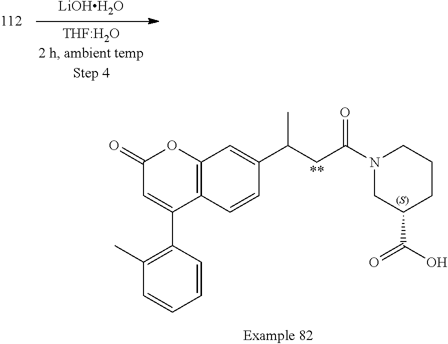

Synthesis of ethyl (3S)-1-(3-(2-oxo-4-(o-tolyl)-2H-chromen-7-yl)butanoyl)piperidine-3-carboxylate, 110 [Step 1]: To a stirred solution of 3-[4-(o-tolyl)-2-oxo-chromen-7-yl]butanoic acid (65, 120 mg, 0.4 mmol) in DCM (5 mL) was added ethyl (3S)-piperidine-3-carboxylate (176 mg, 1 mmol) followed by DIPEA (0.15 mL, 1.1 mmol). T₃P, 50% in EtOAc (0.66 mL, 1 mmol) was added to it at 0° C. and stirred at ambient temperature for 16 h. The reaction mixture was diluted with DCM, washed with water and brine, dried over Na₂SO₄ and concentrated. The product was purified by flash chromatography to afford ethyl (3S)-1-(3-(2-oxo-4-(o-tolyl)-2H-chromen-7-yl)butanoyl)piperidine-3-carboxylate (110, 140 mg). LCMS (ESI) Calcd. For C₂₈H₃₁NO₅: 461, found: [M+H]⁺=462.

Synthesis of chiral ethyl (S)-1-(3-(2-oxo-4-(o-tolyl)-2H-chromen-7-yl)butanoyl)piperidine-3-carboxylate, 111 and 112 [Step 2]: Ethyl (3S)-1-(3-(2-oxo-4-(o-tolyl)-2H-chromen-7-yl)butanoyl)piperidine-3-carboxylate (110, 250 mg, 0.54 mmol) was purified via chiral prep-HPLC and lyophilized to afford Peak 1 as ethyl (S)-1-(3-(2-oxo-4-(o-tolyl)-2H-chromen-7-yl)butanoyl)piperidine-3-carboxylate (111, 120 mg) and Peak 2 as ethyl (S)-1-(3-(2-oxo-4-(o-tolyl)-2H-chromen-7-yl)butanoyl)piperidine-3-carboxylate (112, 70 mg). The absolute stereochemistry of these Examples was not determined.

Ethyl (3S)-1-[3-[4-(o-tolyl)-2-oxo-chromen-7-yl]butanoyl]piperidine-3-carboxylate, 111 (Peak 1): LCMS (ESI) Calcd. for C₂₈H₃₁NO₅: 461, found: [M+H]⁺: 462. ¹H NMR (400 MHz, DMSO-d₆) δ 7.42-7.33 (m, 4H), 7.25-7.18 (m, 2H), 6.88-6.66 (dd, 1H), 6.32 (d, 1H), 4.04-4.00 (m, 2H), 3.77-3.74 (m, 1H), 3.28 (m, 1H), 3.08-2.94 (m, 1H), 2.82-2.79 (m, 1H), 2.66 (m, 1H), 2.32 (m, 1H), 2.11 (s, 3H), 1.96-1.72 (m, 2H), 1.72-1.33 (m, 3H), 1.32-1.05 (m, 7H).

Ethyl (3S)-1-[3-[4-(o-tolyl)-2-oxo-chromen-7-yl]butanoyl]piperidine-3-carboxylate, 112 (Peak 2): LCMS (ESI) Calcd. for C₂₈H₃₁NO₅: 461, found: [M+H]⁺=462. ¹H NMR (400 MHz, DMSO-d₆) δ 7.45-7.39 (m, 3H), 7.35 (t, 1H), 7.25-7.17 (m, 2H), 6.88 (d, 1H), 6.31 (d, 1H), 4.25 (d, 1H), 4.07-4.01 (m, 2H), 3.75 (t, 2H), 3.35-3.26 (m, 1H), 3.03 (m, 1H), 2.77-2.63 (m, 3H), 2.54-2.51 (m, 1H), 2.25-2.13 (m, 1H), 2.11 (s, 3H), 1.86 (m, 1H), 1.69-1.49 (m, 3H), 1.31 (m, 1H), 1.23-1.12 (m, 7H).

Chiral prep-HPLC method: Diastereomer separation was performed on an Agilent 1200 series instrument. Column was a Chiralpak IC (250×20 mm), 5μ, operating at ambient temperature and flow rate of 18.0 mL/min. Mobile phase was a mixture of 80% hexane and 10% ethanol and 10% ethyl acetate, held isocratic for up to 45 min. with wavelength of 280 nm.

Synthesis of (S)-1-(3-(2-oxo-4-(o-tolyl)-2H-chromen-7-yl)butanoyl)piperidine-3-carboxylic acid, Example 81 [Step 3]: To a stirred solution of ethyl (S)-1-(3-(2-oxo-4-(o-tolyl)-2H-chromen-7-yl)butanoyl)piperidine-3-carboxylate (111, 120 mg, 0.3 mmol) in THF (4 mL) and water (1 mL) was added LiOH·H₂O (19 mg, 0.8 mmol) at ambient temperature and stirred for 4 h. The reaction mixture was concentrated under reduced pressure. The product was purified via prep-HPLC and lyophilized to afford(S)-1-((S)-3-(2-oxo-4-(o-tolyl)-2H-chromen-7-yl)butanoyl)piperidine-3-carboxylic acid (Example 81, 28 mg). LCMS (ESI) Calcd. For C₂₆H₂₇NO₅: 433, found: [M+H]⁺=434. ¹H NMR (400 MHz, DMSO-d₆) δ 12.37 (s, 1H), 7.44-7.33 (m, 4H), 7.25-7.15 (m, 2H), 6.88 (d, 1H), 6.30 (s, 1H), 4.38 (m, 1H), 3.80-3.73 (m, 2H), 3.28-3.17 (m, 1H), 2.95-2.71 (m, 2H), 2.68-2.51 (m, 2H), 2.12 (s, 3H), 2.02-1.89 (m, 1H), 1.88-1.74 (m, 1H), 1.72-1.46 (m, 2H), 1.46-1.31 (m, 1H), 1.27-1.20 (m, 3H).

Synthesis of (S)-1-(3-(2-oxo-4-(o-tolyl)-2H-chromen-7-yl)butanoyl)piperidine-3-carboxylic acid, Example 82 [Step 4]: To a stirred solution of ethyl (3S)-1-[3-[4-(o-tolyl)-2-oxo-chromen-7-yl]butanoyl]piperidine-3-carboxylate (112, 70 mg, 0.2 mmol) in THF (4 mL) and water (1 mL) was added LiOH·H₂O (11 mg, 0.5 mmol) and stirred at ambient temperature for 4 h. The reaction mixture was concentrated under reduced pressure. The product was purified via prep-HPLC and lyophilized to afford(S)-1-(3-(2-oxo-4-(o-tolyl)-2H-chromen-7-yl)butanoyl)piperidine-3-carboxylic acid (Example 82, 24 mg). LCMS (ESI) Calcd. For C₂₆H₂₇NO₅: 433, found: [M+H]⁺=434. ¹H NMR (400 MHz, DMSO-d₆) δ 12.37 (m, 1H), 7.45-7.33 (m, 4H), 7.25-7.20 (m, 2H), 6.88 (d, 1H), 6.31 (d, 1H), 4.44-4.19 (m, 1H), 3.75 (d, 2H), 2.98-2.92 (m, 2H), 2.75-2.63 (m, 3H), 2.12-2.07 (m, 34H), 1.87 (m, 1H), 1.75-1.41 (m, 2H), 1.23 (d, 3H).

### Examples 83-84: Synthesis of chiral (S)-1-(2-methyl-3-(2-oxo-4-(o-tolyl)-2H-chromen-7-yl)propanoyl)piperidine-3-carbonitrile

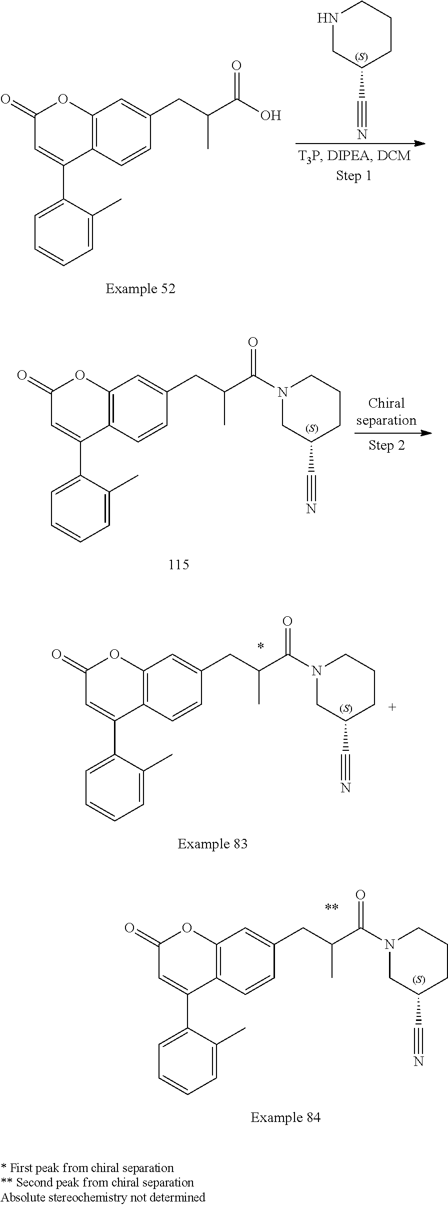

Synthesis of (3S)-1-(2-methyl-3-(2-oxo-4-(o-tolyl)-2H-chromen-7-yl)propanoyl)piperidine-3-carbonitrile, 115 [Step 1]: To a stirred solution of 2-methyl-3-(2-oxo-4-(o-tolyl)-2H-chromen-7-yl)propanoic acid (Example 52, 120 mg, 0.4 mmol) and (S)-piperidine-3-carbonitrile (65 mg, 0.4 mmol) in DCM (5 mL) was added DIPEA (0.2 mL, 1.0 mmol) followed by T₃P, 50% in EtOAc (0.3 mL, 0.6 mmol) at 0° C. under inert atmosphere and stirred at ambient temperature for 2 h. The reaction mixture was diluted with DCM, washed with water and brine, dried over Na₂SO₄ and concentrated under reduced pressure. The product was purified by flash chromatography to afford (3S)-1-(2-methyl-3-(2-oxo-4-(o-tolyl)-2H-chromen-7-yl)propanoyl)piperidine-3-carbonitrile (115, 100 mg).

Synthesis of chiral (S)-1-(2-methyl-3-(2-oxo-4-(o-tolyl)-2H-chromen-7-yl)propanoyl)piperidine-3-carbonitrile, Examples 83 and 84 [Step 2]: (3S)-1-(2-methyl-3-(2-oxo-4-(o-tolyl)-2H-chromen-7-yl)propanoyl)piperidine-3-carbonitrile (115, 100 mg) was purified by NP chiral prep-HPLC and lyophilized to afford Peak 1 as (S)-1-(2-methyl-3-(2-oxo-4-(o-tolyl)-2H-chromen-7-yl)propanoyl)piperidine-3-carbonitrile (Example 83, 27 mg) and Peak 2 as (S)-1-(2-methyl-3-(2-oxo-4-(o-tolyl)-2H-chromen-7-yl)propanoyl)piperidine-3-carbonitrile (Example 84, 22 mg). Chirality of both enantiomers was taken arbitrarily.

Peak 1: (S)-1-(2-methyl-3-(2-oxo-4-(o-tolyl)-2H-chromen-7-yl)propanoyl)piperidine-3-carbonitrile, Example 83: LCMS (ESI) Calcd. for C₂₆H₂₆N₂O₃: 414, found: [M+H]⁺=415. ¹H NMR (400 MHz, DMSO-d₆) δ 7.45-7.35 (m, 4H), 7.25-7.23 (d, 1H), 7.13-7.09 (m, 1H), 6.87-6.85 (m, 1H), 6.31 (s, 1H), 3.86-3.84 (m, 1H), 3.65-3.61 (m, 2H), 3.39 (m, 1H), 3.25-3.23 (m, 1H), 3.04-3.03 (m, 1H), 2.97-2.95 (m, 1H), 2.70-2.67 (m, 1H), 2.10 (s, 3H), 1.85-1.80 (m, 2H), 1.48 (m, 1H), 1.23 (m, 1H), 1.04-0.95 (m, 3H).

Peak 2: (S)-1-(2-methyl-3-(2-oxo-4-(o-tolyl)-2H-chromen-7-yl)propanoyl)piperidine-3-carbonitrile, Example 84: LCMS (ESI) Calcd. for C₂₆H₂₆N₂O₃: 414, found: [M+H]⁺=415. ¹H NMR (400 MHz, DMSO-d₆) δ 7.45-7.35 (m, 4H), 7.25-7.23 (d, 1H), 7.13-7.09 (m, 1H), 6.87-6.85 (m, 1H), 6.31 (s, 1H), 3.86-3.84 (m, 1H), 3.65-3.61 (m, 2H), 3.39 (m, 1H), 3.25-3.23 (m, 1H), 3.04-3.03 (m, 1H), 2.97-2.95 (m, 1H), 2.70-2.67 (m, 1H), 2.10 (s, 3H), 1.85-1.80 (m, 2H), 1.48 (m, 1H), 1.23 (m, 1H), 1.04-0.95 (m, 3H).

Chiral prep-HPLC method: Chiral separation was performed on an Agilent 1200 series instrument. Column was a Chiralpak IC (250×20 mm), 5μ, operating at ambient temperature and flow rate of 18.0 mL/min. Mobile phase was a mixture of 60% hexane, 20% dichloromethane and 20% ethanol, held isocratic for up to 26 min. with wavelength of 282 nm.

### Examples 85-86: Synthesis of chiral 7-(2-methyl-3-((S)-3-(methylsulfonyl)piperidin-1-yl)-3-oxopropyl)-4-(o-tolyl)-2H-chromen-2-one

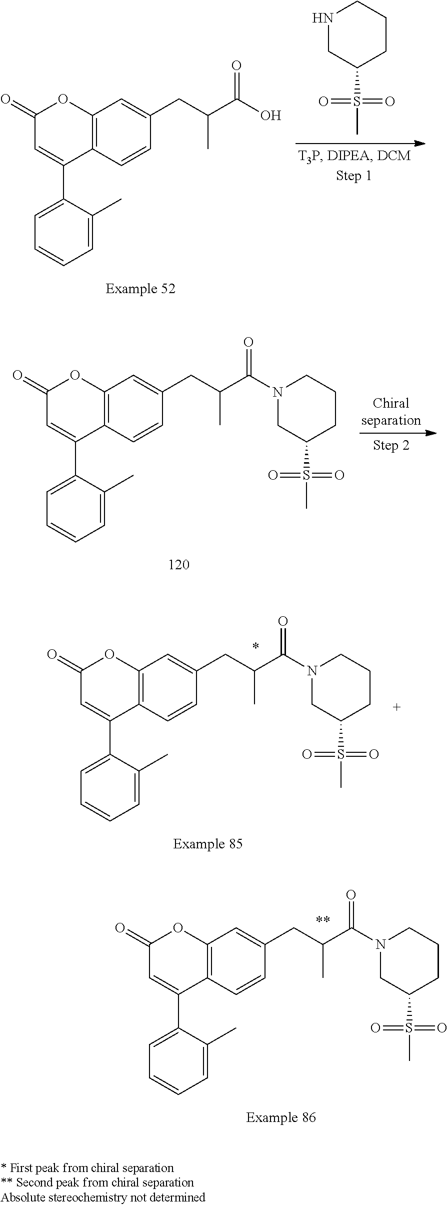

Synthesis of 7-(2-methyl-3-((S)-3-(methylsulfonyl)piperidin-1-yl)-3-oxopropyl)-4-(o-tolyl)-2H-chromen-2-one, 120 [Step 1]: To a stirred solution of 2-methyl-3-(2-oxo-4-(o-tolyl)-2H-chromen-7-yl)propanoic acid (Example 52, 100 mg, 0.3 mmol) and (S)-3-(methylsulfonyl)piperidine (60 mg, 0.4 mmol) in DCM (5 mL) was added DIPEA (0.2 mL, 0.9 mmol) followed by T₃P, 50% in ethyl acetate (295 mg, 0.5 mmol) at 0° C. and stirred at ambient temperature for 2 h. It was diluted with DCM and washed with water and brine, dried over Na₂SO₄ and concentrated under reduced pressure. The product was purified via flash chromatography to afford 7-(2-methyl-3-((S)-3-(methylsulfonyl)piperidin-1-yl)-3-oxopropyl)-4-(o-tolyl)-2H-chromen-2-one (120, 68 mg).

Synthesis of chiral 7-(2-methyl-3-((S)-3-(methylsulfonyl)piperidin-1-yl)-3-oxopropyl)-4-(o-tolyl)-2H-chromen-2-one, Examples 85 and 86 [Step 2]: 7-(2-methyl-3-((S)-3-(methylsulfonyl)piperidin-1-yl)-3-oxopropyl)-4-(o-tolyl)-2H-chromen-2-one (120, 68 mg) was separated via NP chiral prep-HPLC and lyophilized to afford Peak 1 as 7-(2-methyl-3-((S)-3-(methylsulfonyl)piperidin-1-yl)-3-oxopropyl)-4-(o-tolyl)-2H-chromen-2-one (Example 85, 33 mg) and Peak 2 as 7-(2-methyl-3-((S)-3-(methylsulfonyl)piperidin-1-yl)-3-oxopropyl)-4-(o-tolyl)-2H-chromen-2-one (Example 86, 22 mg).

Peak 1:7-(2-methyl-3-((S)-3-(methylsulfonyl)piperidin-1-yl)-3-oxopropyl)-4-(o-tolyl)-2H-chromen-2-one, Example 85: LCMS (ESI) Calcd. for C₂₆H₂₉NO₅S: 467., found: [M+H]⁺=468. ¹H NMR (400 MHz, DMSO-d₆ at 100° C.) δ 7.45-7.33 (m, 4H), 7.25 (d, 1H), 7.14-7.09 (m, 1H), 6.90-6.84 (m, 1H), 6.31 (s, 1H), 4.64 (d, 1H), 4.20 (d, 1H), 3.86 (d, 1H), 3.25-3.22 (m, 1H), 3.01 (s, 1H), 2.95 (d, 3H), 2.79 (d, 1H), 2.72-2.66 (m, 1H), 2.10 (s, 3H), 1.97 (d, 1H), 1.67 (s, 2H), 1.23 (s, 1H), 1.03 (s, 3H), 0.85 (d, 1H).

Peak 2:7-(2-methyl-3-((S)-3-(methylsulfonyl)piperidin-1-yl)-3-oxopropyl)-4-(o-tolyl)-2H-chromen-2-one, Example 86: LCMS (ESI) Calcd. for C₂₆H₂₉NO₅S: 467, found: [M+H]⁺=468. ¹H NMR (400 MHz, DMSO-d₆ at 100° C.) δ 7.45-7.31 (m, 4H), 7.24-7.23 (m, 1H), 7.10-7.09 (m, 1H), 6.90-6.88 (m, 1H), 6.32 (s, 1H), 4.72 (d, 1H), 4.01 (d, 1H), 3.22 (d, 1H), 2.91 (d, 3H), 2.82 (d, 1H), 2.70-2.66 (m, 1H), 2.10 (s, 3H), 1.94 (d, 1H), 1.82 (d, 1H), 1.71 (d, 2H), 1.36 (s, 1H), 1.26 (s, 2H), 1.06 (d, 3H).

Chiral prep-HPLC method: Chiral separation was performed on an Agilent 1200 series instrument. Column was a REFLECT I-Cellulose C (250×21 mm), 5μ, operating at ambient temperature and flow rate of 21.0 mL/min. Mobile phase was a mixture of 70% hexane, 15% ethyl acetate and 15% ethanol, held isocratic for up to 25 min. with wavelength of 280 nm.

### Examples 87-88: Synthesis of chiral ethyl (S)-1-(2-methyl-3-(2-oxo-4-(o-tolyl)-2H-chromen-7-yl)propanoyl)piperidine-3-carboxylate

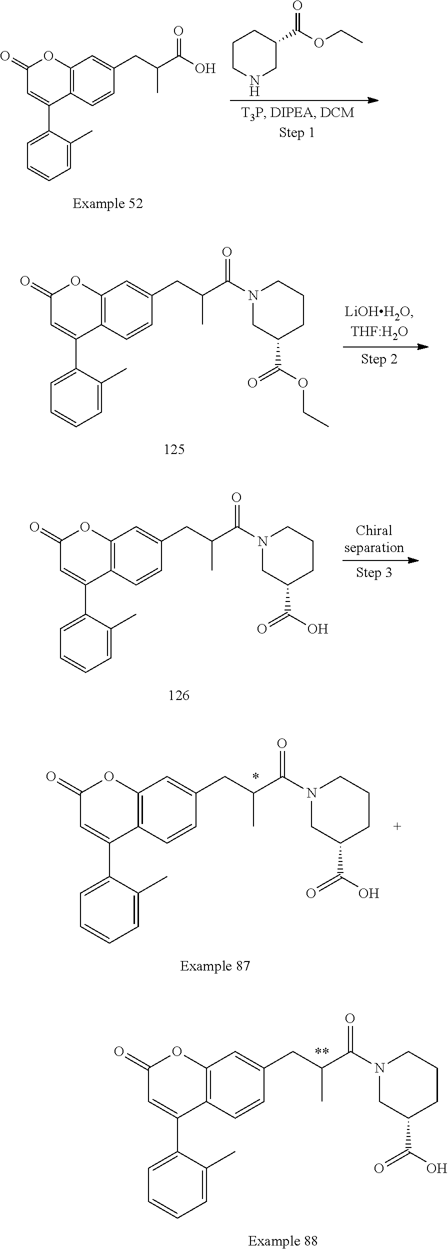

Synthesis of ethyl (3S)-1-(2-methyl-3-(2-oxo-4-(o-tolyl)-2H-chromen-7-yl)propanoyl)piperidine-3-carboxylate, 125 [Step 1]: To a stirred solution of 2-methyl-3-(2-oxo-4-(o-tolyl)-2H-chromen-7-yl)propanoic acid (Example 52, 350 mg, 1.1 mmol) and ethyl (S)-piperidine-3-carboxylate (205 mg, 1.3 mmol) in DCM (10 mL) was added DIPEA (0.6 mL, 3.3 mmol) followed by T₃P, 50% in EtOAc (1 mL, 1.6 mmol) at 0° C. and stirred at ambient temperature for 2 h. The reaction mixture was diluted with DCM and extracted with water. The organic layer was collected, washed with brine, dried over Na₂SO₄ and concentrated to afford ethyl (3S)-1-(2-methyl-3-(2-oxo-4-(o-tolyl)-2H-chromen-7-yl)propanoyl)piperidine-3-carboxylate (125, 400 mg). The product was used directly for next step. LCMS (ESI) Calcd. for C₂₈H₃₁NO₅: 461, found: [M+H]⁺=462.

Synthesis of (3S)-1-(2-methyl-3-(2-oxo-4-(o-tolyl)-2H-chromen-7-yl)propanoyl)piperidine-3-carboxylic acid, 126 [Step 2]: To a stirred solution of ethyl (3S)-1-(2-methyl-3-(2-oxo-4-(o-tolyl)-2H-chromen-7-yl)propanoyl)piperidine-3-carboxylate (125, 500 mg, 1.1 mmol) in THF (10 mL) and water (2 mL) mixture was added LiOH. H₂O (135 mg, 3.3 mmol) and stirred at ambient temperature for 4 h. The reaction mass was neutralized with 10% citric acid and extracted with EtOAc. The combined organic layer was washed with water and brine, dried over Na₂SO₄ and concentrated under reduced pressure. The product was purified by flash column chromatography to afford (3S)-1-(2-methyl-3-(2-oxo-4-(o-tolyl)-2H-chromen-7-yl)propanoyl)piperidine-3-carboxylic acid (126, 82 mg).

Synthesis of chiral ethyl (S)-1-(2-methyl-3-(2-oxo-4-(o-tolyl)-2H-chromen-7-yl)propanoyl)piperidine-3-carboxylate, Examples 87 and 88 [Step 3]: (3S)-1-(2-methyl-3-(2-oxo-4-(o-tolyl)-2H-chromen-7-yl)propanoyl)piperidine-3-carboxylic acid (126, 80 mg) was purified by NP chiral prep-HPLC and lyophilized to afford Peak 1 as ethyl (S)-1-(2-methyl-3-(2-oxo-4-(o-tolyl)-2H-chromen-7-yl)propanoyl)piperidine-3-carboxylate (Example 87, 30 mg) and Peak 2 as ethyl (S)-1-(2-methyl-3-(2-oxo-4-(o-tolyl)-2H-chromen-7-yl)propanoyl)piperidine-3-carboxylate (Example 88, 15 mg). Chirality of both enantiomers was taken arbitrarily. 0324 Peak 1: Ethyl(S)-1-(2-methyl-3-(2-oxo-4-(o-tolyl)-2H-chromen-7-yl)propanoyl)piperidine-3-carboxylate, Example 89: LCMS (ESI) Calcd. for C₂₆H₂₇NO₅: 433, found: [M+H]⁺=434. ¹H NMR (400 MHz, DMSO-d₆) (VT-100° C.) δ 12.0 (s, 1H), 7.45-7.29 (m, 4H), 7.24 (d, 1H), 7.10 (s, 1H), 6.90 (d, 1H), 6.25 (s, 1H), 4.15 (m, 1H), 3.80 (d, 1H), 3.54 (s, 1H), 3.23-3.18 (m, 2H), 2.74-2.67 (m, 1H), 2.54 (s, 1H), 2.32 (d, 1H), 2.12 (s, 3H), 1.85 (s, 1H), 1.60 (d, 2H), 1.27 (s, 1H), 1.06 (d, 3H).

Peak 2: Ethyl(S)-1-((R)-2-methyl-3-(2-oxo-4-(o-tolyl)-2H-chromen-7-yl)propanoyl)piperidine-3-carboxylate, Example 90: LCMS (ESI) Calcd. for C₂₆H₂₇NO₅: 433, found: [M+H]⁺=434. ¹H NMR (400 MHz, DMSO-d₆) (VT-100° C.) δ 12.0 (s, 1H), 7.45-7.29 (m, 4H), 7.24 (d, 1H), 7.10 (s, 1H), 6.90 (d, 1H), 6.25 (s, 1H), 4.15 (m, 1H), 3.80 (d, 1H), 3.54 (s, 1H), 3.23-3.18 (m, 2H), 2.74-2.67 (m, 1H), 2.54 (s, 1H), 2.32 (d, 1H), 2.12 (s, 3H), 1.85 (s, 1H), 1.60 (d, 2H), 1.27 (s, 1H), 1.06 (d, 3H).

Chiral prep-HPLC method: Chiral separation was performed on an Agilent 1200 series instrument. Column was a CHIRALPAK AY-H (250×21 mm), 5μ, operating at ambient temperature and flow rate of 21.0 mL/min. Mobile phase was a mixture of 70% hexane, 25% propanol, 5% methanol and 0.1% TFA, held isocratic for up to 25 min. with detection at 282 nm wavelength.

### Examples 89-90: Synthesis of chiral (S)-1-(2-methyl-3-(2-oxo-4-(o-tolyl)-2H-chromen-7-yl)propanoyl)piperidine-3-carboxamide

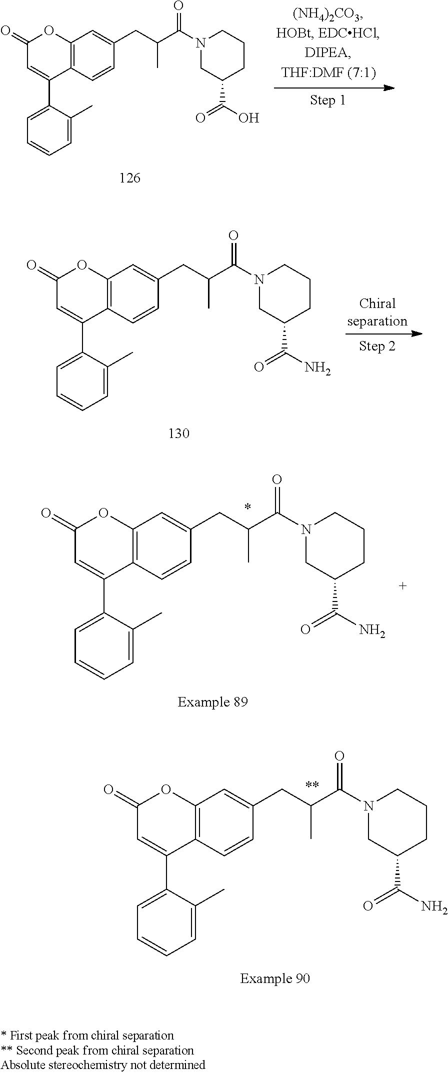 [SMILES: Cc1ccccc1-c1cc(=O)oc2cc(CC(C)C(=O)N3CCC[C@H](C(=O)O)C3)ccc12 ; Cc1ccccc1-c1cc(=O)oc2cc(CC(C)C(=O)N3CCC[C@H](C(N)=O)C3)ccc12 ; Cc1ccccc1-c1cc(=O)oc2cc(CC(C)C(=O)N3CCC[C@H](C(N)=O)C3)ccc12 ; Cc1ccccc1-c1cc(=O)oc2cc(CC(C)C(=O)N3CCC[C@H](C(N)=O)C3)ccc12]

Synthesis of (3S)-1-(2-methyl-3-(2-oxo-4-(o-tolyl)-2H-chromen-7-yl)propanoyl)piperidine-3-carboxamide, 130 [Step 1]: To a stirred solution of (3S)-1-(2-methyl-3-(2-oxo-4-(o-tolyl)-2H-chromen-7-yl)propanoyl)piperidine-3-carboxylic acid (126, 200 mg, 0.5 mmol) in THF (4 mL) and DMF (1 mL) were added HOBt (95 mg, 0.7 mmol), EDC·HCl (133 mg, 0.7 mmol) and DIPEA (0.3 mL, 1.8 mmol) followed by (NH₄)₂CO₃ (175 mg, 1.8 mmol) and stirred at ambient temperature for 17 h. The reaction mixture was diluted with water and extracted with EtOAc. The organic layer was washed with brine, dried over Na₂SO₄, and concentrated. The product was purified by flash column chromatography to afford (3S)-1-(2-methyl-3-(2-oxo-4-(o-tolyl)-2H-chromen-7-yl)propanoyl)piperidine-3-carboxamide (130, 115 mg).

Synthesis of chiral (S)-1-(2-methyl-3-(2-oxo-4-(o-tolyl)-2H-chromen-7-yl)propanoyl)piperidine-3-carboxamide, Examples 89 and 90 [Step 2]: (3S)-1-(2-methyl-3-(2-oxo-4-(o-tolyl)-2H-chromen-7-yl)propanoyl)piperidine-3-carboxamide (130, 115 mg) was purified by NP chiral prep-HPLC and lyophilized to afford Peak 1 as (S)-1-(2-methyl-3-(2-oxo-4-(o-tolyl)-2H-chromen-7-yl)propanoyl)piperidine-3-carboxamide (Example 89, 40 mg) and Peak 2 as (S)-1-(2-methyl-3-(2-oxo-4-(o-tolyl)-2H-chromen-7-yl)propanoyl)piperidine-3-carboxamide (Example 90, 34 mg). Chirality of both enantiomers was taken arbitrarily.

Peak 1: (S)-1-((S)-2-methyl-3-(2-oxo-4-(o-tolyl)-2H-chromen-7-yl)propanoyl)piperidine-3-carboxamide, Example 89: LCMS (ESI) Calcd. for C₂₆H₂₈N₂O₄: 432, found: [M+H]⁺=433. ¹H NMR (400 MHz, DMSO-d₆) δ 7.43-7.24 (m, 6H), 7.13-7.08 (m, 1H), 6.90-6.80 (m, 2H), 6.31 (s, 1H), 4.38 (dd, 1H), 3.87-3.84 (m, 1H), 3.28-3.21 (m, 1H), 2.96 (q, 2H), 2.67 (d, 1H), 2.53 (s, 1H), 2.23 (s, 1H), 2.10 (s, 3H), 1.93-1.83 (m, 1H), 1.70-1.35 (m, 3H), 1.01 (d, 3H).

Peak 2: (S)-1-(2-methyl-3-(2-oxo-4-(o-tolyl)-2H-chromen-7-yl)propanoyl)piperidine-3-carboxamide, Example 90: LCMS (ESI) Calcd. for C₂₆H₂₈N₂O₄:432, found: [M+H]⁺=433. ¹H NMR (400 MHz, DMSO-d₆) δ 7.45-7.33 (m, 4H), 7.26 (d, 2H), 7.13-7.08 (m, 1H), 6.90-6.80 (m, 2H), 6.31 (s, 1H), 4.38 (dd, 1H), 3.87-3.84 (m, 1H), 3.28-3.21 (m, 1H), 2.96 (q, 2H), 2.67 (d, 1H), 2.53 (s, 1H), 2.23 (s, 1H), 2.10 (s, 3H), 1.93-1.83 (m, 1H), 1.70-1.35 (m, 3H), 1.01 (d, 3H).

Chiral prep-HPLC method: Chiral separation was performed on an Agilent 1200 series instrument. Column was a CHIRALPAK AY-H (250×21 mm), 5μ, operating at ambient temperature and flow rate of 21.0 mL/min. Mobile phase was a mixture of 80% hexane, 20% propanol and 0.1% isopropylamine, held isocratic for up to 40 min. with detection at 220 nm wavelength.

### Example 91: Synthesis of 7-(1-aminoethyl)-4-(o-tolyl)-2H-chromen-2-one

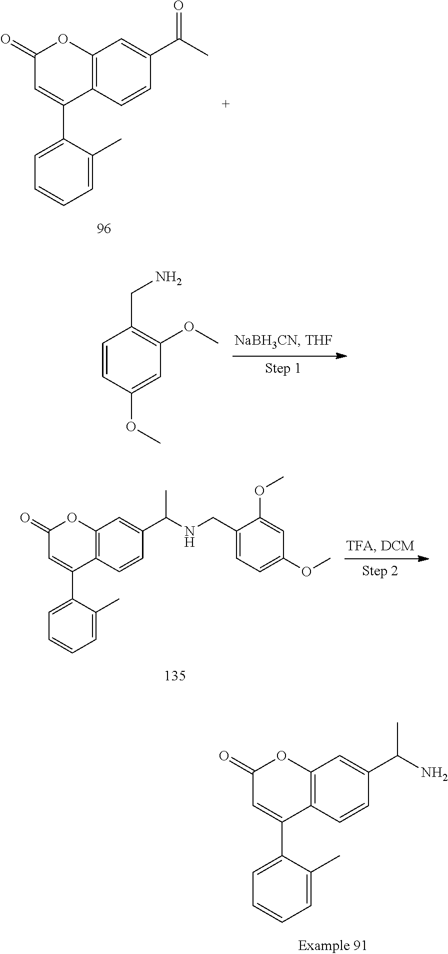 [SMILES: CC(=O)c1ccc2c(-c3ccccc3C)cc(=O)oc2c1 ; COc1ccc(CN)c(OC)c1 ; COc1ccc(CNC(C)c2ccc3c(-c4ccccc4C)cc(=O)oc3c2)c(OC)c1 ; Cc1ccccc1-c1cc(=O)oc2cc(C(C)N)ccc12]

Synthesis of 7-(1-((2,4-dimethoxybenzyl)amino)ethyl)-4-(o-tolyl)-2H-chromen-2-one, 135 [Step 1]: To a stirred solution of 7-acetyl-4-(o-tolyl)-2H-chromen-2-one (96, 50 mg, 0.2 mmol) and (2,4-dimethoxyphenyl)methanamine (0.03 mL, 0.2 mmol) in THF (1 mL), acetic acid (0.1 mL) was added and stirred at ambient temperature for 15 min. Then the reaction mixture was cooled and NaBH₃CN (25 mg, 0.4 mmol) was added and the reaction mixture was stirred at ambient temperature for 16 h. TLC and LCMS showed full consumption of starting material with formation of new polar spot. The reaction mixture was diluted with EtOAc and washed with NaHCO₃, water and brine. The organic phase was dried over Na₂SO₄, filtered and evaporated. The product was purified by column chromatography over silica gel using EtOAc to yield 7-(1-((2,4-dimethoxybenzyl)amino)ethyl)-4-(o-tolyl)-2H-chromen-2-one (135, 60 mg). LCMS (ESI) Calcd. for C₂₇H₂₇NO₄: 429, found: [M+H]⁺=430.

Synthesis of 7-(1-aminoethyl)-4-(o-tolyl)-2H-chromen-2-one, Example 91 [Step 2]: In a sealed tube 7-(1-((2,4-dimethoxybenzyl)amino)ethyl)-4-(o-tolyl)-2H-chromen-2-one (135, 60 mg, 0.14 mmol) was taken in TFA (1.0 mL, 13.1 mmol) and heated at 80° C. for 16 h. TLC and LCMS showed full conversion of starting material with formation of new polar spot. The reaction mixture was concentrated under reduced pressure and the residue was submitted for reverse phase prep-HPLC purification. The fraction obtained from reverse phase prep-HPLC was evaporated to yield 7-(1-aminoethyl)-4-(o-tolyl)-2H-chromen-2-one (Example 91, 34 mg). LCMS (ESI) Calcd. for C₁₈H₁₇NO₂: 279, found: [M+H]⁺=280. ¹H NMR (400 MHz, DMSO-d₆) δ 7.50 (d, 1H), 7.46-7.40 (m, 2H), 7.35 (t, 1H), 7.31-7.24 (m, 2H), 6.91-6.89 (m, 1H), 6.31 (s, 1H), 4.06 (q, 1H), 2.11 (s, 3H), 1.25 (d, 3H). Two amine protons are merged with solvent peak.

### Examples 92-93: Synthesis of chiral (S)—N-methyl-1-(2-methyl-3-(2-oxo-4-(o-tolyl)-2H-chromen-7-yl)propanoyl)piperidine-3-carboxamide

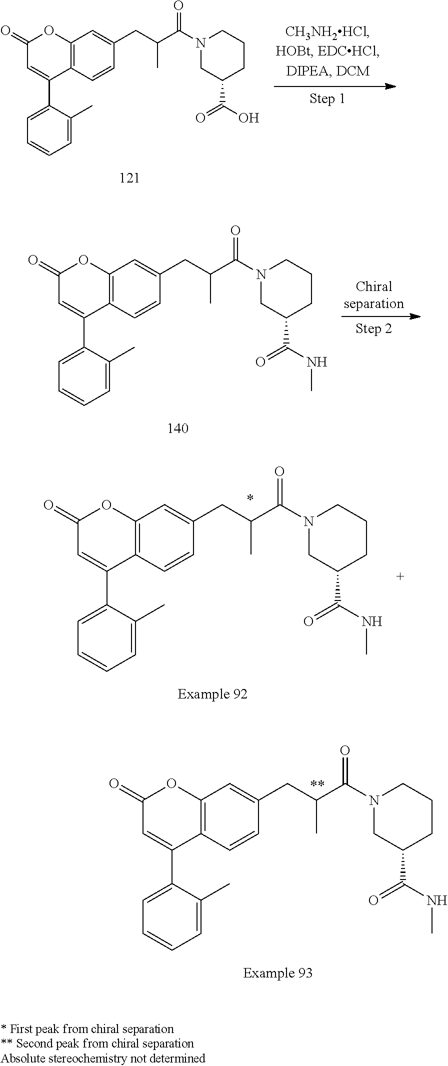 [SMILES: Cc1ccccc1-c1cc(=O)oc2cc(CC(C)C(=O)N3CCC[C@H](C(=O)O)C3)ccc12 ; CNC(=O)[C@H]1CCCN(C(=O)C(C)Cc2ccc3c(-c4ccccc4C)cc(=O)oc3c2)C1 ; CNC(=O)[C@H]1CCCN(C(=O)C(C)Cc2ccc3c(-c4ccccc4C)cc(=O)oc3c2)C1 ; CNC(=O)[C@H]1CCCN(C(=O)C(C)Cc2ccc3c(-c4ccccc4C)cc(=O)oc3c2)C1]

Synthesis of (3S)—N-methyl-1-(2-methyl-3-(2-oxo-4-(o-tolyl)-2H-chromen-7-yl)propanoyl)piperidine-3-carboxamide, 140 [Step 1]: To a stirred solution of (3S)-1-(2-methyl-3-(2-oxo-4-(o-tolyl)-2H-chromen-7-yl)propanoyl)piperidine-3-carboxylic acid (121, 200 mg, 0.5 mmol) in DCM (5 mL) were added HOBt (95 mg, 0.7 mmol), EDC·HCl (135 mg, 0.7 mmol) and DIPEA (0.3 mL, 1.8 mmol) followed by CH₃NH₂·HCl (125 mg, 1.8 mmol) and stirred at ambient temperature for 17 h. The reaction mixture was diluted with water and extracted with EtOAc. The organic layer was washed with brine, dried over Na₂SO₄ and concentrated. The product was purified by flash column chromatography to afford (3S)—N-methyl-1-(2-methyl-3-(2-oxo-4-(o-tolyl)-2H-chromen-7-yl)propanoyl)piperidine-3-carboxamide (140, 180 mg).

Synthesis of chiral (S)—N-methyl-1-(2-methyl-3-(2-oxo-4-(o-tolyl)-2H-chromen-7-yl)propanoyl)piperidine-3-carboxamide, Examples 92-93 [Step 2]: (3S)—N-methyl-1-(2-methyl-3-(2-oxo-4-(o-tolyl)-2H-chromen-7-yl)propanoyl)piperidine-3-carboxamide (140, 180 mg) was purified by NP chiral prep-HPLC and lyophilized to afford Peak 1 as (S)—N-methyl-1-(2-methyl-3-(2-oxo-4-(o-tolyl)-2H-chromen-7-yl)propanoyl)piperidine-3-carboxamide (Example 92, 65 mg) and Peak 2 as (S)—N-methyl-1-(2-methyl-3-(2-oxo-4-(o-tolyl)-2H-chromen-7-yl)propanoyl)piperidine-3-carboxamide (Example 93, 45 mg). Chirality of both enantiomers were taken arbitrarily. 0336 Peak 1: (S)—N-methyl-1-(2-methyl-3-(2-oxo-4-(o-tolyl)-2H-chromen-7-yl)propanoyl)piperidine-3-carboxamide, Example 92: LCMS (ESI) Calcd. for C₂₇H₃₀N₂O₄: 446, found: [M+H]⁺=447. ¹H NMR (400 MHz, DMSO-d₆) δ 7.83-7.74 (m, 1H), 7.45-7.40 (m, 2H), 7.36 (d, 2H), 7.25 (d, 1H), 7.14-7.08 (m, 1H), 6.90-6.84 (m, 1H), 6.31 (s, 1H), 4.38 (dd, 1H), 3.88 (d, 1H), 3.31-3.23 (m, 1H), 2.99 (q, 2H), 2.67 (d, 1H), 2.50 (d, 3H), 2.23 (s, 1H), 2.10 (s, 3H), 1.92 (d, 1H), 1.81 (d, 1H), 1.65-1.50 (m, 3H), 1.01 (d, 3H).

Peak 2: (S)—N-methyl-1-(2-methyl-3-(2-oxo-4-(o-tolyl)-2H-chromen-7-yl)propanoyl)piperidine-3-carboxamide, Example 93: LCMS (ESI) Calcd. for C₂₇H₃₀N₂O₄: 446, found: [M+H]⁺=447. ¹H NMR (400 MHz, DMSO-d₆) δ 7.79-7.67 (m, 1H), 7.45-7.40 (m, 2H), 7.36 (d, 2H), 7.25 (d, 1H), 7.12-7.10 (m, 1H), 6.90 (m, 1H), 6.31 (s, 1H), 4.38 (dd, 1H), 3.88 (d, 1H), 3.31-3.23 (m, 1H), 2.99 (q, 2H), 2.67 (d, 1H), 2.50 (d, 3H), 2.23 (s, 1H), 2.10 (s, 3H), 1.92 (d, 1H), 1.81 (d, 1H), 1.65-1.50 (m, 3H), 1.01 (d, 3H).

Chiral prep-HPLC method: Chiral separation was performed on an Agilent 1200 series instrument. Column was a CHIRALPAK AY-H (250×21 mm), 5μ, operating at ambient temperature and flow rate of 21.0 mL/min. Mobile phase was a the mixture of 75% hexane, 25% ethanol, held isocratic for up to 25 min. with wavelength of 220 nm.

### Examples 94-95: Synthesis of chiral 1-(2-methyl-3-(2-oxo-4-(o-tolyl)-2H-chromen-7-yl)propanoyl)piperidine-4-carboxylic acid

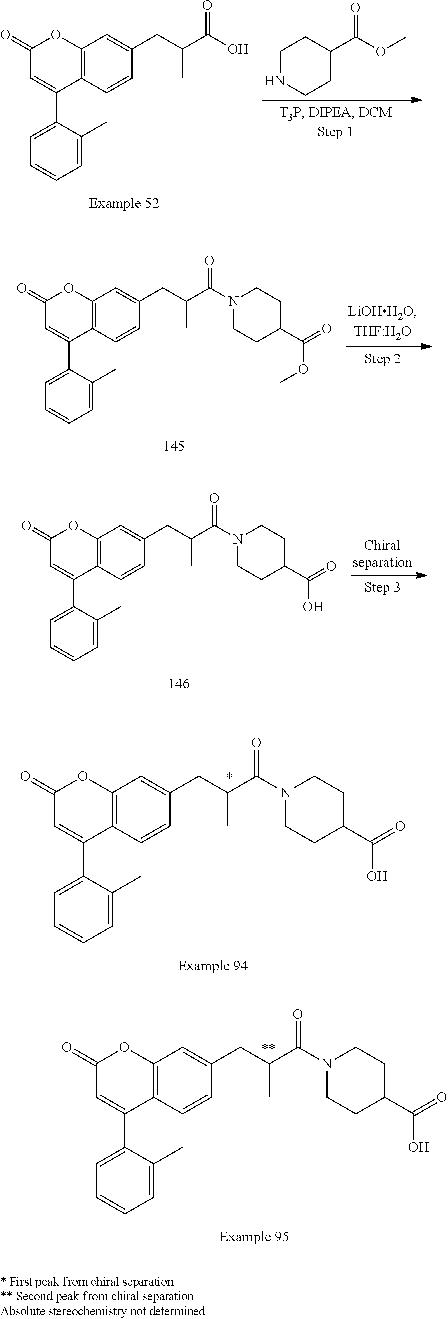

Synthesis of methyl 1-(2-methyl-3-(2-oxo-4-(o-tolyl)-2H-chromen-7-yl)propanoyl)piperidine-4-carboxylate, 145 [Step 1]: To a stirred solution of 2-methyl-3-(2-oxo-4-(o-tolyl)-2H-chromen-7-yl)propanoic acid (Example 52, 130 mg, 0.4 mmol) and methyl piperidine-4-carboxylate (70 mg, 0.5 mmol) in DCM (5 mL) was added DIPEA (0.2 mL, 1.2 mmol) followed by T₃P, 50% in EtOAc (0.4 mL, 0.6 mmol) at 0° C. and stirred at ambient temperature for 2 h. The reaction mixture was diluted with DCM and washed with water. The organic layer was collected and washed with brine, dried over Na₂SO₄ and concentrated to afford methyl 1-(2-methyl-3-(2-oxo-4-(o-tolyl)-2H-chromen-7-yl)propanoyl)piperidine-4-carboxylate (145, 210 mg). The product was used for next step. LCMS (ESI) Calcd. for C₂₇H₂₉NO₅: 447, found: [M+H]⁺=448.

Synthesis of 1-(2-methyl-3-(2-oxo-4-(o-tolyl)-2H-chromen-7-yl)propanoyl)piperidine-4-carboxylic acid, 146 [Step 2]: To a stirred solution of methyl 1-(2-methyl-3-(2-oxo-4-(o-tolyl)-2H-chromen-7-yl)propanoyl)piperidine-4-carboxylate (145, 210 mg, 0.5 mmol) in THF (5 mL) and water (1 mL) mixture was added LiOH·H₂O (60 mg, 1.4 mmol) and stirred at ambient temperature for 4 h. The reaction mixture was concentrated under reduced pressure and neutralized with 10% aqueous citric acid solution, washed with water and brine, dried over Na₂SO₄ and concentrated under reduced pressure again. The product was purified by flash column chromatography to afford 1-(2-methyl-3-(2-oxo-4-(o-tolyl)-2H-chromen-7-yl)propanoyl)piperidine-4-carboxylic acid (146, 130 mg).

Synthesis of chiral 1-(2-methyl-3-(2-oxo-4-(o-tolyl)-2H-chromen-7-yl)propanoyl)piperidine-4-carboxylic acid, Examples 94 and 95 [Step 3]: 1-(2-methyl-3-(2-oxo-4-(o-tolyl)-2H-chromen-7-yl)propanoyl)piperidine-4-carboxylic acid (146, 130 mg) was purified by NP chiral prep-HPLC and lyophilized to afford Peak 1 as 1-(2-methyl-3-(2-oxo-4-(o-tolyl)-2H-chromen-7-yl)propanoyl)piperidine-4-carboxylic acid (Example 94, 35 mg) and Peak 2 as 1-(2-methyl-3-(2-oxo-4-(o-tolyl)-2H-chromen-7-yl)propanoyl)piperidine-4-carboxylic acid (Example 95, 13 mg). Chirality of both enantiomers were taken arbitrarily.

Peak 1:1-(2-methyl-3-(2-oxo-4-(o-tolyl)-2H-chromen-7-yl)propanoyl)piperidine-4-carboxylic acid, Example 94: LCMS (ESI) Calcd. for C₂₆H₂₇NO₅: 433, found: [M+H]⁺=434. ¹H NMR (400 MHz, DMSO-d₆) δ 12.3 (s, 1H), 7.42-7.23 (m, 5H), 7.12 (d, 1H), 6.87 (t, 1H), 6.31 (s, 1H), 4.33 (dd, 1H), 3.86-3.76 (m, 2H), 3.20-3.17 (m, 1H), 2.96-2.83 (m, 2H), 2.73-2.66 (m, 1H), 2.32 (s, 1H), 2.09 (s, 3H), 1.70 (d, 1H), 1.54 (s, 1H), 1.17 (d, 1H), 1.12 (d, 1H), 1.01 (d, 3H).

Peak 2:1-(2-methyl-3-(2-oxo-4-(o-tolyl)-2H-chromen-7-yl)propanoyl)piperidine-4-carboxylic acid, Example 95: LCMS (ESI) Calcd. for C₂₆H₂₇NO₅: 433, found: [M+H]⁺=434. ¹H NMR (400 MHz, DMSO-d₆) δ 12.3 (s, 1H), 7.40-7.23 (m, 5H), 7.12 (d, 1H), 6.87 (t, 1H), 6.31 (s, 1H), 4.33 (dd, 1H), 3.86-3.76 (m, 2H), 3.20 (d, 1H), 2.96-2.83 (m, 2H), 2.73-2.66 (m, 1H), 2.32 (s, 1H), 2.09 (s, 3H), 1.70 (d, 1H), 1.54 (s, 1H), 1.17 (d, 1H), 1.12 (d, 1H), 1.01 (d, 3H).

Chiral prep-HPLC method: Chiral separation was performed on an Agilent 1200 series instrument. Column was a CHIRALPAK AY-H (250×21 mm), 5μ, operating at ambient temperature and flow rate of 21.0 mL/min. Mobile phase was a mixture of 80% hexane, 20% propanol and 0.1% isopropylamine, held isocratic for up to 40 min. with detection at 220 nm wavelength.

### Example 96: Synthesis of N-(1-(2-oxo-4-(o-tolyl)-2H-chromen-7-yl)ethyl)acetamide

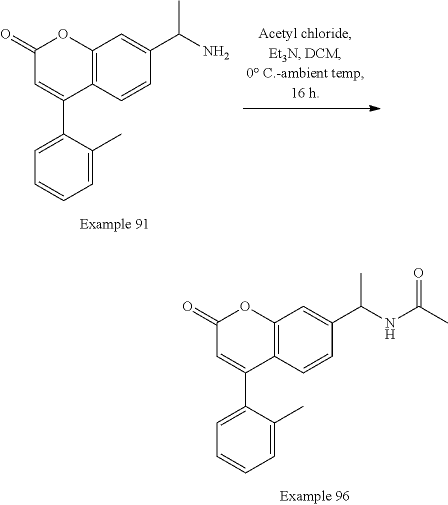 [SMILES: Cc1ccccc1-c1cc(=O)oc2cc(C(C)N)ccc12 ; CC(=O)NC(C)c1ccc2c(-c3ccccc3C)cc(=O)oc2c1]

Synthesis of N-(1-(2-oxo-4-(o-tolyl)-2H-chromen-7-yl)ethyl)acetamide, Example 96 [Step 1]: To a stirred solution of 7-(1-aminoethyl)-4-(o-tolyl)-2H-chromen-2-one (Example 91, 200 mg, 0.7 mmol) in DCM (5 mL), acetyl chloride (0.08 mL, 1.1 mmol) and triethylamine (0.25 mL, 1.8 mmol) was added at 0° C. and the reaction mixture was stirred at ambient temperature for 16 h. TLC showed full conversion of starting material with formation of new non polar spot. LCMS showed the formation of desired product. The reaction mixture was diluted with DCM and washed with water and brine. The reaction mixture was then dried over Na₂SO₄, filtered and evaporated. The product was purified by reverse phase prep-HPLC and lyophilized to yield N-(1-(2-oxo-4-(o-tolyl)-2H-chromen-7-yl)ethyl)acetamide (Example 96, 110 mg). LCMS (ESI): Calcd. for C₂₀H₁₉NO₃: 321; [M+H]⁺=322. ¹H NMR (400 MHz, DMSO-d₆) δ 8.40 (d, 1H), 7.46-7.33 (m, 4H), 7.25-7.21 (m, 2H), 6.92 (d, 1H), 6.34 (s, 1H), 4.97-4.93 (m, 1H), 2.12 (s, 3H), 1.85 (s, 3H), 1.35 (d, 3H).

### Examples 97-98: Synthesis of chiral (S)-1-(2-methyl-3-(2-oxo-4-(o-tolyl)-2H-chromen-7-yl)propanoyl)piperidine-3-sulfonamide

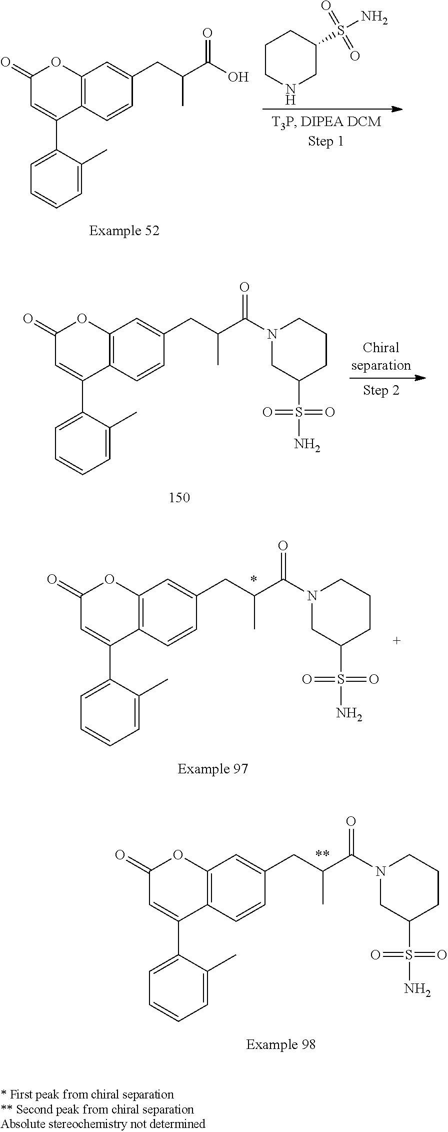

Synthesis of (3S)-1-(2-methyl-3-(2-oxo-4-(o-tolyl)-2H-chromen-7-yl)propanoyl)piperidine-3-sulfonamide, 150 [Step 1]: To a stirred solution of 2-methyl-3-[4-(o-tolyl)-2-oxo-chromen-7-yl]propanoic acid (Example 52, 200 mg, 0.6 mmol) and (3S)-piperidine-3-sulfonamide (125 mg, 0.7 mmol) in DCM (5 mL) was added DIPEA (0.3 mL, 1.9 mmol) followed by T₃P 50% in EtOAc (0.6 mL, 0.9 mmol) at 0° C. and stirred at ambient temperature for 4 h. The reaction mixture was diluted with DCM and extracted with water, The organic layer was collected, washed with brine, dried over Na₂SO₄, and concentrated. The product was purified by flash column chromatography to afford (3S)-1-(2-methyl-3-(2-oxo-4-(o-tolyl)-2H-chromen-7-yl)propanoyl)piperidine-3-sulfonamide (150, 190 mg).

Synthesis of chiral (S)-1-(2-methyl-3-(2-oxo-4-(o-tolyl)-2H-chromen-7-yl)propanoyl)piperidine-3-sulfonamide, Examples 97-98 [Step 2]: (3S)-1-(2-methyl-3-(2-oxo-4-(o-tolyl)-2H-chromen-7-yl)propanoyl)piperidine-3-sulfonamide (150, 190 mg) was purified by chiral prep-HPLC and lyophilized to afford Peak 1 as (S)-1-(2-methyl-3-(2-oxo-4-(o-tolyl)-2H-chromen-7-yl)propanoyl)piperidine-3-sulfonamide (Example 97, 65 mg) and Peak 2 as (S)-1-(2-methyl-3-(2-oxo-4-(o-tolyl)-2H-chromen-7-yl)propanoyl)piperidine-3-sulfonamide (Example 98, 70 mg). Chirality of both diastereomers were taken arbitrarily.

Peak 1: (S)-1-(2-methyl-3-(2-oxo-4-(o-tolyl)-2H-chromen-7-yl)propanoyl)piperidine-3-sulfonamide, Example 97: LCMS (ESI) Calcd. for C₂₅H₂₈N₂O₅S: 468, found: [M+H]⁺=469. ¹H NMR (400 MHz, DMSO-d₆) (VT-100° C.): δ 7.45-7.31 (m, 4H), 7.24 (d, 1H), 7.10 (d, 1H), 6.90 (d, 1H), 6.61 (s, 2H), 6.25 (s, 1H), 4.67 (d, 1H), 4.01 (s, 1H), 3.23-3.15 (m, 1H), 3.04-3.00 (m, 1H), 2.80-2.67 (m, 4H), 2.13 (s, 4H), 1.76-1.62 (m, 2H), 1.08 (d, 4H).

Peak 2: (S)-1-(2-methyl-3-(2-oxo-4-(o-tolyl)-2H-chromen-7-yl)propanoyl)piperidine-3-sulfonamide, Example 98: LCMS (ESI) Calcd. for C₂₅H₂₈N₂Os₅: 468, found: [M+H]⁺=469. ¹H NMR (400 MHz, DMSO-d₆) (VT-100° C.): δ 7.43-7.29 (m, 4H), 7.25 (d, 1H), 7.11 (d, 1H), 6.91 (d, 1H), 6.59 (s, 2H), 6.25 (s, 1H), 4.55 (d, 1H), 4.04 (s, 1H), 3.20-3.14 (m, 1H), 3.02-3.00 (m, 1H), 2.75-2.66 (m, 4H), 2.13 (s, 4H), 1.80 (d, 1H), 1.77-1.64 (m, 1H), 1.27 (s, 1H), 1.08 (d, 3H).

Chiral prep-HPLC method: Prep-HPLC chiral method: Chiral separation was performed on an Agilent 1200 series instrument. Column was a CHIRALPAK IG (250×20 mm), 5μ, operating at ambient temperature and a flow rate of 18.0 mL/min. Mobile phase was a mixture of 50% hexane, 25% of dichloromethane and 25% of ethanol, held isocratic for up to 20 min. with detection at 284 nm wavelength.

### Examples 99-100: Synthesis of chiral (S)-1-(2-methyl-3-(2-oxo-4-(o-tolyl)-2H-chromen-7-yl)propanoyl)pyrrolidine-3-carboxylic acid

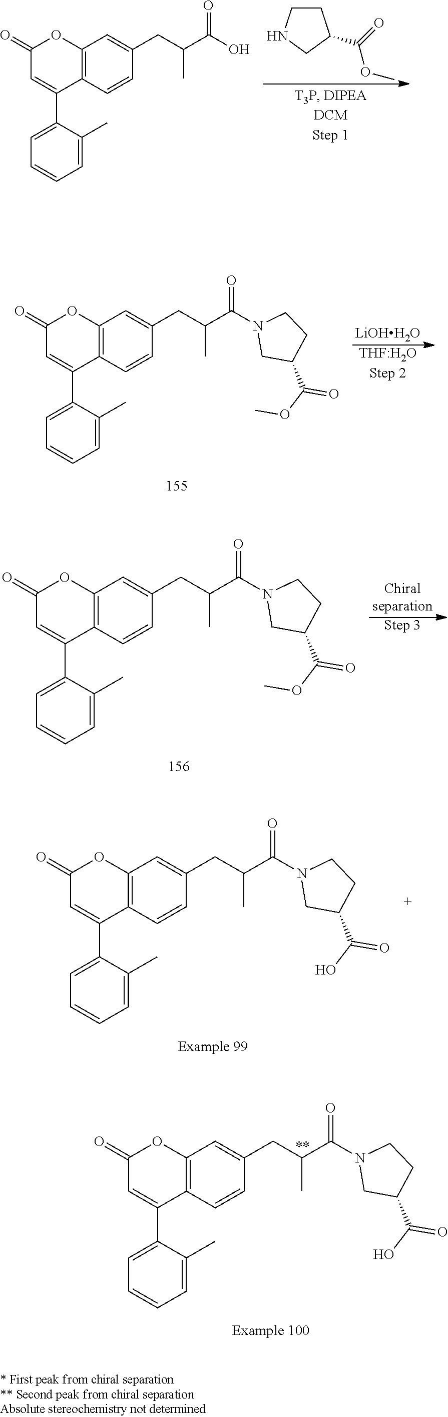

Synthesis of methyl(3S)-1-(2-methyl-3-(2-oxo-4-(o-tolyl)-2H-chromen-7-yl)propanoyl)pyrrolidine-3-carboxylate, 155 [Step 1]: To a stirred solution of 2-methyl-3-[4-(o-tolyl)-2-oxo-chromen-7-yl]propanoic acid (Example 52, 200 mg, 0.6 mmol) and methyl (3S)-pyrrolidine-3-carboxylate·HCl (124 mg, 0.7 mmol) in DCM (5 mL) was added DIPEA (0.3 mL, 1.9 mmol) followed by T₃P 50% in EtOAc (0.6 mL, 0.9 mmol) at 0° C. and stirred at ambient temperature for 2 h. The reaction mixture was diluted with DCM and extracted with water. The organic layer was collected, washed with brine, dried over Na₂SO₄, and concentrated to afford the product methyl(3S)-1-(2-methyl-3-(2-oxo-4-(o-tolyl)-2H-chromen-7-yl)propanoyl)pyrrolidine-3-carboxylate (155, 250 mg). This product was used for next step. LCMS (ESI) Calcd. for C₂₆H₂₇NO₅: 433, found [M+H]⁺=434.

Synthesis of (3S)-1-(2-methyl-3-(2-oxo-4-(o-tolyl)-2H-chromen-7-yl)propanoyl)pyrrolidine-3-carboxylic acid, 156 [Step 2]: To a stirred solution of methyl(35)-1-[2-methyl-3-[4-(o-tolyl)-2-oxo-chromen-7-yl]propanoyl]pyrrolidine-3-carboxylate (155, 250 mg, 0.6 mmol) in THF (5 mL) and water (2 mL) was added LiOH·H₂O (73 mg, 1.7 mmol) and stirred at ambient temperature for 4 h. The reaction mixture was partitioned between EtOAc and 10% aq. solution of citric acid. The organic layer was collected and washed with water and brine, dried over Na₂SO₄, and concentrated under reduced pressure. This product was purified by flash column chromatography to afford (35)-1-(2-methyl-3-(2-oxo-4-(o-tolyl)-2H-chromen-7-yl)propanoyl)pyrrolidine-3-carboxylic acid (156, 180 mg).

Synthesis of chiral (S)-1-(2-methyl-3-(2-oxo-4-(o-tolyl)-2H-chromen-7-yl)propanoyl)pyrrolidine-3-carboxylic acid, Examples 99 and 100 [Step 3]: (35)-1-(2-methyl-3-(2-oxo-4-(o-tolyl)-2H-chromen-7-yl)propanoyl)pyrrolidine-3-carboxylic acid (156, 180 mg) was purified by chiral prep-HPLC and lyophilized to afford Peak 1 as (S)-1-(2-methyl-3-(2-oxo-4-(o-tolyl)-2H-chromen-7-yl)propanoyl)pyrrolidine-3-carboxylic acid (Example 99, 32 mg) and Peak 2 as (S)-1-(2-methyl-3-(2-oxo-4-(o-tolyl)-2H-chromen-7-yl)propanoyl)pyrrolidine-3-carboxylic acid (Example 100, 23 mg). Chirality of both diastereomers were taken arbitrarily.

Peak 1: (S)-1-(2-methyl-3-(2-oxo-4-(o-tolyl)-2H-chromen-7-yl)propanoyl)pyrrolidine-3-carboxylic acid, Example 99: LCMS (ESI) Calcd. for C₂₅H₂₅NO₅: 419, found [M+H]⁺=420. ¹H NMR (400 MHz, DMSO-d₆) (VT-100° C.) δ 11.93 (s, 1H), 7.45-7.29 (m, 4H), 7.25 (d, 1H), 7.10 (d, 1H), 6.90 (d, 1H), 6.24 (s, 1H), 3.54-3.44 (m, 4H), 3.31-3.25 (m, 2H), 2.73 (m, 2H), 2.13 (s, 3H), 2.03 (d, 2H), 1.07 (d, 3H).

Peak 2: (S)-1-(2-methyl-3-(2-oxo-4-(o-tolyl)-2H-chromen-7-yl)propanoyl)pyrrolidine-3-carboxylic acid, Example 100: LCMS (ESI) Calcd. for C₂₅H₂₅NO₅: 419, found [M+H]⁺=420. ¹H NMR (400 MHz, DMSO-d₆) (VT-100° C.) δ 11.93 (s, 1H), 7.45-7.29 (m, 4H), 7.25 (d, 1H), 7.11 (d, 1H), 6.90 (d, 1H), 6.24 (s, 1H), 3.51-3.40 (m, 4H), 2.96 (d, 3H), 2.73 (m, 1H), 2.13 (s, 3H), 2.04 (d, 2H), 1.07 (d, 3H).

Prep-HPLC chiral method: Chiral separation was performed on an Agilent 1200 series instrument. Column was a CHIRALPAK AY-H (250×21 mm), 5μ, operating at ambient temperature and flow rate of 21.0 mL/min. Mobile phase was a mixture of 0.1% trifluoroacetic acid in a mixture of 70% hexane and 30% isopropyl alcohol, held isocratic for up to 30 min. with detection at 210 nm wavelength.

### Example 101: Synthesis of N-methyl-N-(2-oxo-4-phenyl-2H-chromen-7-yl)glycine

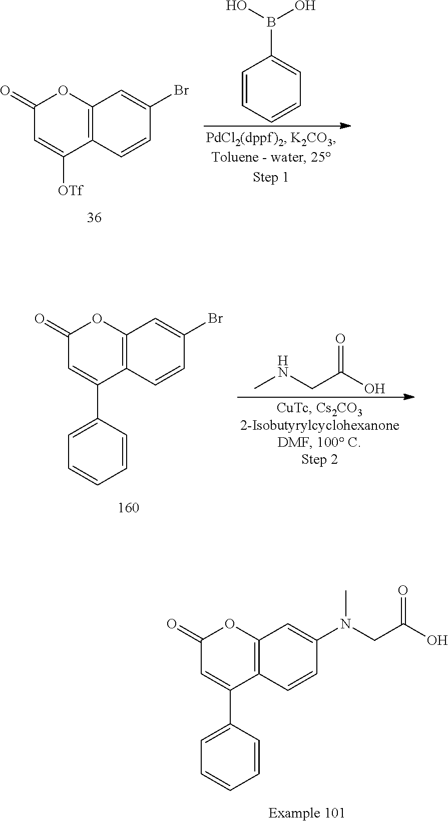

Synthesis of 7-bromo-4-phenyl-2H-chromen-2-one, 160 [Step 1]: An oven dried round bottom flask was charged with 7-bromo-2-oxo-2H-chromen-4-yl trifluoromethanesulfonate (36, 300 mg, 0.8 mmol) followed by phenyl boronic acid (90 mg, 0.7 mmol) and toluene (5 mL). Potassium carbonate (245 mg, 1.8 mmol) in water (1.5 mL) was added to it, and the mixture was degassed by bubbling argon gas for 10 min. Then Pd (dppf) Cl₂ (60 mg, 80 μmol) was added, and the reaction mixture stirred at 25° C. for 1 h. The reaction mixture was partitioned between EtOAc and water. The organic phase was collected, washed with brine, dried over anhydrous Na₂SO₄ and concentrated under reduced pressure. The product was purified on silica gel column using EtOAc/Hexanes to afford 7-bromo-4-phenyl-2H-chromen-2-one (160, 160 mg). LCMS (ESI) Calcd. for C₁₅H₉BrO₂: 300.0, found: [M+H]⁺=301.2.

Synthesis of N-methyl-N-(2-oxo-4-phenyl-2H-chromen-7-yl)glycine, Example 101 [Step 2]: To an oven dried sealed tube was charged with 7-bromo-4-phenyl-2H-chromen-2-one (160, 150 mg, 0.5 mmol) followed by N-methylglycine (53 mg, 0.6 mmol) and DMF (2 mL). Cesium carbonate (325 mg, 1.0 mmol), copper (II) thiophen-2-carboxylate (20 mg, 99 μmol) and 2-isobutyrylcyclohexanone (67 mg, 4.0 mmol) were added to the reaction mixture, which was flushed with argon and stirred at 100° C. for 16 h. The reaction mixture was filtered and partitioned between EtOAc and water. The water layer was collected and acidified to pH 3 using 2N HCl. Then organic layer was extracted using MeOH-DCM and washed with cold brine, dried over anhydrous Na₂SO₄, and concentrated under reduced pressure. The product was purified by reverse phase prep-HPLC to afford N-methyl-N-(2-oxo-4-phenyl-2H-chromen-7-yl)glycine (Example 101, 60 mg). LCMS (ESI) Calcd. for C₁₈H₁₅NO₄: 309, found: [M+H]⁺=310. ¹H NMR (400 MHz, DMSO-d₆): δ 7.54-7.47 (m, 5H), 7.18-7.16 (m, 1H), 6.66-6.59 (m, 2H), 5.94 (s, 1H) 4.06 (s, 2H), 3.07 (s, 3H).

### Example 102: Synthesis of O-methyl-N-(2-oxo-4-(o-tolyl)-2H-chromen-7-yl) serine

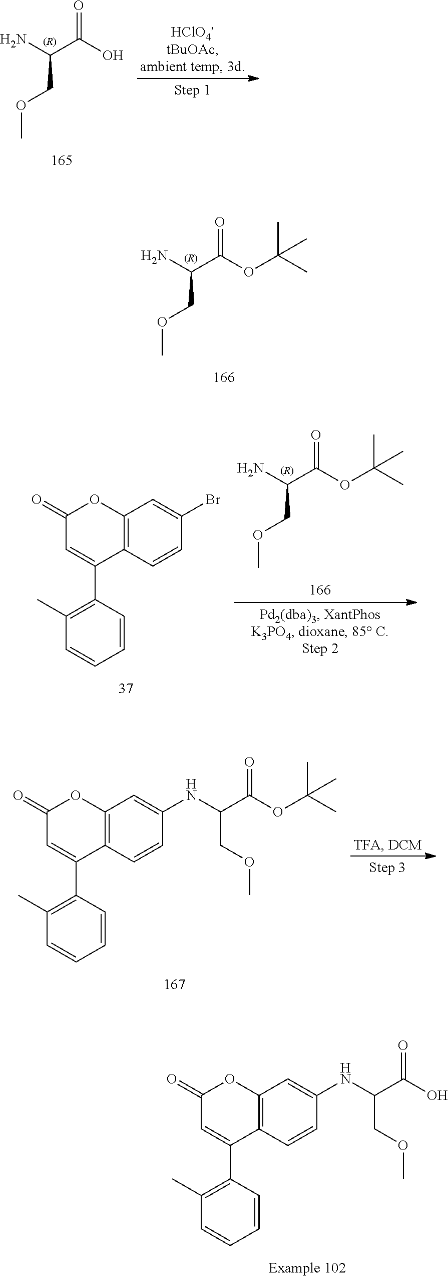

Synthesis of tert-butyl O-methyl-D-serinate, 166 [Step 1]: To a stirring solution of (2R)-2-amino-3-methoxy-propanoic acid (165, 1.00 g, 8.39 mmol) in tert-butyl acetate (10 mL, 74.2 mmol) was added perchloric acid (0.72 mL, 8.39 mmol) and stirred for 3 days at ambient temperature. 2M NaOH solution was then added to the reaction mixture slowly and stirred for 10 minutes. The reaction mixture was diluted with ethyl acetate. The organic layer was washed with brine, dried over Na₂SO₄, and concentrated under reduced pressure. The product (166, 700 mg) was used for next step. ¹H NMR (400 MHz, DMSO-d₆): δ 8.22 (s, 2H), 4.19 (s, 1H), 3.74-3.69 (m, 1H), 3.65-3.62 (m, 1H) 1.46 (s, 9H), O-methyl peak merged with solvent peak 3.32.

Synthesis of tert-butyl O-methyl-N-(2-oxo-4-(o-tolyl)-2H-chromen-7-yl) serinate, 167 [Step 2]: An oven dried sealed tube was charged with 7-bromo-4-(o-tolyl)-2H-chromen-2-one (37, 900 mg, 3.0 mmol) followed by tert-butyl O-methyl-D-serinate (166, 550 mg, 3.1 mmol), and 1,4-dioxane (8 mL). Potassium phosphate (1.2 g, 5.7 mmol) was added to the mixture and degassed with argon for 5 min. XantPhos (330 mg, 0.6 mmol) and Pd₂(dba)₃ (261 mg, 0.3 mmol) were added and stirred at 85° C. for 10 h. The reaction mixture was filtered and partitioned between EtOAc and water. The organic layer was collected, washed with brine, dried over anhydrous Na₂SO₄, and concentrated under reduced pressure. The product was purified on silica gel column chromatography to afford tert-butyl O-methyl-N-(2-oxo-4-(o-tolyl)-2H-chromen-7-yl) serinate (167, 400 mg). LCMS (ESI) Calcd. for C₂₄H₂₇NO₅: 409, found: [M+H]⁺=410.

Synthesis of O-methyl-N-(2-oxo-4-(o-tolyl)-2H-chromen-7-yl) serine, Example 102 [Step 3]: An oven dried round bottom flask was charged with tert-butyl O-methyl-N-(2-oxo-4-(o-tolyl)-2H-chromen-7-yl) serinate (167, 25 mg, 61 μmol) DCM (1 mL). Trifluoroacetic acid (47 μL, 0.6 mmol) were added to it and flushed with argon and stirred at ambient temperature for 6 h. The reaction mixture was filtered and partitioned between DCM and water. DCM layer was collected and washed with brine, dried over anhydrous Na₂SO₄ and concentrated under reduced pressure. The product was purified on silica gel column using reverse phase prep-HPLC to afford O-methyl-N-(2-oxo-4-(o-tolyl)-2H-chromen-7-yl) serine (Example 102, 9.0 mg). LCMS (ESI) Calcd. for C₂₀H₁₉NO₅: 353, found: [M+H]⁺=354. ¹H NMR (400 MHz, DMSO-d₆): δ 7.40-7.30 (m, 3H), 7.20-7.18 (m, 1H), 6.67-6.59 (m, 4H), 5.83 (s, 1H) 4.13 (s, 1H), 3.76-3.66 (m, 2H), 3.31 (s, 3H), 2.14 (s, 3H).

### Examples 103-104: Synthesis of diastereomeric(S)-1-(O-methyl-N-(2-oxo-4-(o-tolyl)-2H-chromen-7-yl)-seryl)piperidine-3-carboxylic acid

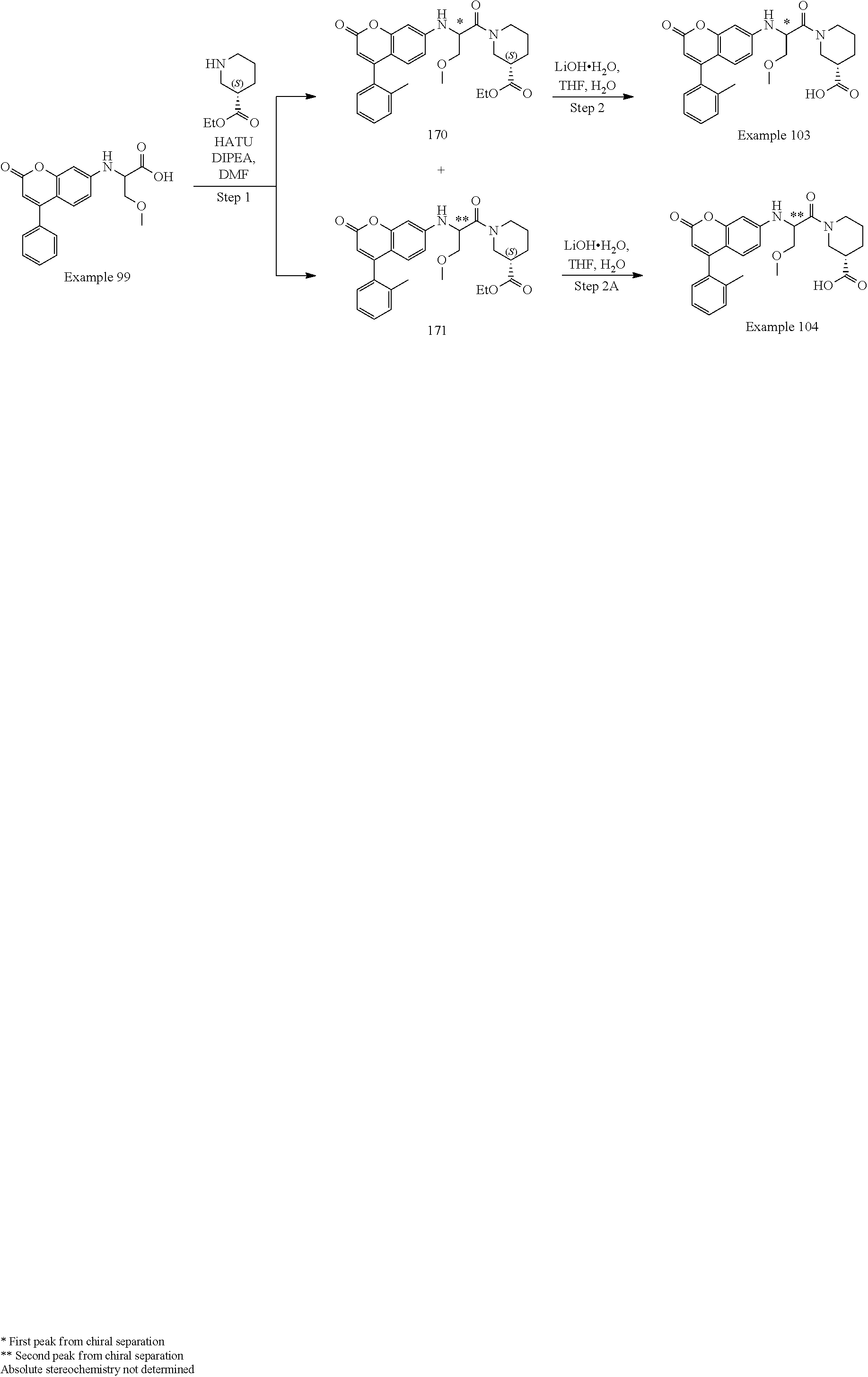

Synthesis of diastereomeric ethyl (S)-1-(O-methyl-N-(2-oxo-4-(o-tolyl)-2H-chromen-7-yl)-seryl)piperidine-3-carboxylate, 170 and 171 [Step 1]: An oven dried round bottom flask was charged with ethyl (S)-piperidine-3-carboxylate (0.1 mL, 0.6 mmol) followed by DMF (3 mL), O-methyl-N-(2-oxo-4-phenyl-2H-chromen-7-yl) serine (Example 99, 200 mg, 0.6 mmol). HATU (237 mg, 0.6 mmol) and DIPEA (0.1 mL, 0.8 mmol) were added to the mixture and flushed with argon and stirred at 25° C. for 16 h. The reaction mixture was filtered and partitioned between EtOAc and water. The collected organic layer was washed with cold brin, dried over anhydrous Na₂SO₄, and concentrated under reduced pressure. Two diastereomers were separated from the product using PREP (NP).

Chiral prep-HPLC method: Prep-HPLC chiral method: Chiral separation was performed on an Agilent 1200 series instrument. Column was a CHIRALPAK IG (250×20 mm), 5μ, operating at ambient temperature and flow rate of 18.0 mL/min. Mobile phase was a mixture of 60% hexane, 20% of dichloromethane and 20% of ethanol, held isocratic for up to 25 min. with detection at 358 nm wavelength.

Peak 1: Ethyl(S)-1-(O-methyl-N-(2-oxo-4-(o-tolyl)-2H-chromen-7-yl)-seryl)piperidine-3-carboxylate (170, 75 mg). LCMS (ESI) Calcd. for C₂₈H₃₂N₂O₆: 492, found: [M+H]⁺=493.

Peak 2: Ethyl(S)-1-(O-methyl-N-(2-oxo-4-(o-tolyl)-2H-chromen-7-yl)-seryl)piperidine-3-carboxylate (171, 50 mg). LCMS (ESI) Calcd. for C₂₈H₃₂N₂O₆: 492, found: [M+H]⁺=493.

Synthesis of diastereomeric(S)-1-(O-methyl-N-(2-oxo-4-(o-tolyl)-2H-chromen-7-yl)-seryl)piperidine-3-carboxylic acid, Example 103 [Step 2]: An oven dried round bottom flask was charged with of ethyl (S)-1-(O-methyl-N-(2-oxo-4-(o-tolyl)-2H-chromen-7-yl)-seryl)piperidine-3-carboxylate (170, 75 mg, 0.2 mmol) followed by THF (4.5 mL) and water (0.5 mL). Lithium hydroxide (18 mg, 0.8 mmol) was added to it and stirred at 25° C. for 6 h. Water and DCM were added to the reaction mixture. The aqueous phase was collected and acidified using 1N HCl slowly until pH of reached nearly 5-6. The mixture was extracted using 15% MeOH-DCM. Combined organic layer was dried over Na₂SO₄ and concentrated under reduced pressure. The product was purified using reverse phase prep-HPLC to afford(S)-1-(O-methyl-N-(2-oxo-4-(o-tolyl)-2H-chromen-7-yl)-seryl)piperidine-3-carboxylic acid (Example 103, 19 mg). ¹H NMR (400 MHz, DMSO-d₆): δ 7.40-7.30 (m, 3H), 7.20-7.18 (m, 1H), 6.68-6.63 (m, 3H), 6.48 (s, 1H), 5.84 (s, 1H) 4.79-4.15 (m, 1H), 4.15 (s, 1H), 3.90-3.87 (m, 1H), 3.62-3.58 (m, 2H) 3.30 (s, 3H), 3.17-3.12 (m, 2H) 2.40-2.33 (m, 1H), 2.14 (s, 3H), 1.98-1.95 (m, 1H), 1.68-1.45 (m, 2H), 1.18 (s, 1H). LCMS (ESI) Calcd. For C₂₆H₂₈N₂O₆:464, found: [M+H]⁺=465.

Synthesis of (S)-1-(O-methyl-N-(2-oxo-4-(o-tolyl)-2H-chromen-7-yl)-D-seryl)piperidine-3-carboxylic acid, Example 104 [Step 2A]: An oven dried round bottom flask was charged with ethyl (S)-1-(O-methyl-N-(2-oxo-4-(o-tolyl)-2H-chromen-7-yl)-seryl)piperidine-3-carboxylate (171, 50 mg, 0.1 mmol) followed by THF (4.5 mL) and water (0.5 mL). Lithium hydroxide (12 mg, 0.5 mmol) was added to it and stirred at 25° C. for 6 h. Water and DCM were added to the reaction mixture. The aqueous phase was collected and acidified using 1N HCl slowly until pH of reached nearly 5-6. The reaction mixture was extracted using 15% MeOH-DCM. Combined organic layer was dried over Na₂SO₄ and concentrated under reduced pressure. The product was purified using reverse phase prep-HPLC to afford(S)-1-(O-methyl-N-(2-oxo-4-(o-tolyl)-2H-chromen-7-yl)-seryl)piperidine-3-carboxylic acid (Example 104, 17 mg). ¹H NMR (400 MHz, DMSO-d₆): δ 7.39-7.30 (m, 3H), 7.20-7.18 (m, 1H), 6.66-6.62 (m, 3H), 6.55 (s, 1H), 5.85 (s, 1H) 4.76-4.74 (m, 1H), 4.22 (s, 1H), 4.02-3.99 (m, 1H), 3.64-3.58 (m, 2H) 3.30 (s, 3H), 2.34-2.30 (m, 1H), 2.14 (s, 3H), 2.08-1.97 (m, 1H), 1.70-1.62 (m, 2H), 1.18 (s, 1H), two peaks merged with solvent peak. LCMS (ESI) Calcd. for C₂₆H₂₈N₂O₆:464, found: [M+H]⁺=465.

### Example 105: Synthesis N-(4-(2-chloro-4-fluorophenyl)-2-oxo-2H-chromen-7-yl)-N-methylglycine

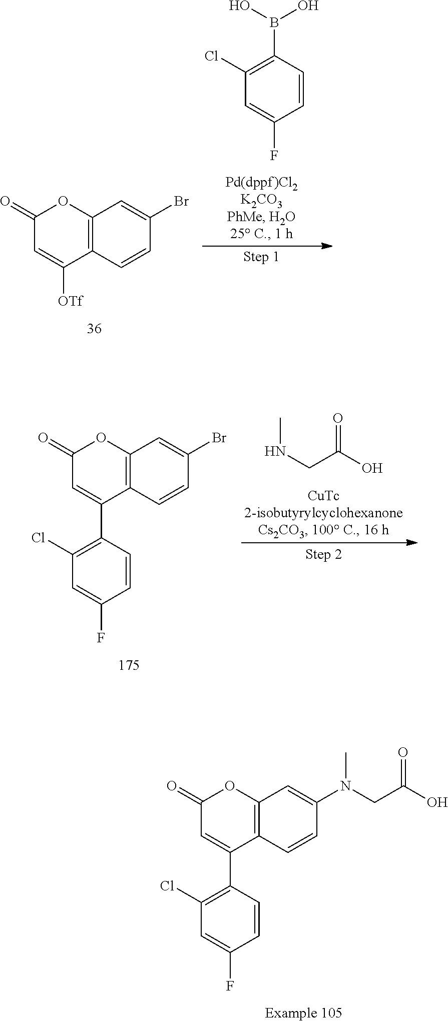

Synthesis 7-bromo-4-(2-chloro-4-fluorophenyl)-2H-chromen-2-one, 175 [Step 1]: A mixture of 7-bromo-2-oxo-2H-chromen-4-yl trifluoromethanesulfonate (36, 300 mg, 0.8 mmol), 2-chloro-4-fluorophenyl) boronic acid (126 mg, 0.7 mmol) and potassium carbonate (244 mg, 1.8 mmol) in toluene (5 mL)-water (1.5 mL) was degassed by bubbling argon for 10 minutes. Pd (dppf) Cl₂ (59 mg, 0.1 mmol) was added, and the mixture was stirred at 25° C. After 1 h., the reaction mixture was partitioned between water and EtOAc. Layers were separated, and the aqueous phase was extracted with EtOAc. The combined organic phase was washed with brine, dried over anhydrous Na₂SO₄, filtered, and concentrated under reduced pressure. The product was purified on a silica gel column to afford 7-bromo-4-(2-chloro-4-fluorophenyl)-2H-chromen-2-one (175, 180 mg). LCMS (ESI) Calcd. for C₁₅H₇BrClFO₂: 352, found: [M+H]⁺=353. ¹H NMR (400 MHz, DMSO-d₆): δ 7.86-7.84 (m, 1H), 7.74-7.72 (m, 1H), 7.60-7.57 (m, 1H), 7.52-7.46 (m, 2H), 6.99-6.97 (m, 1H), 6.59 (s, 1H).

Synthesis of N-(4-(2-chloro-4-fluorophenyl)-2-oxo-2H-chromen-7-yl)-N-methylglycine, Example 105 [Step 2]: A sealed tube was charged with methylglycine (60 mg, 0.7 mmol), 7-bromo-4-(2-chloro-4-fluorophenyl)-2H-chromen-2-one (175, 160 mg, 0.4 mmol), copper thiophene 2-carboxylate (17 mg, 0.1 mmol), 2-isobutyrylcyclohexanone (61 mg, 0.4 mmol), Cs₂CO₃ (295 mg, 0.9 mmol) and DMF (3 mL). The reaction vessel was flushed with argon, and then capped tightly. The reaction tube was immersed into a heating block preheated to 100° C. and stirred for 16 h. After cooling to ambient temperature, the reaction mixture was quenched with water, acidified to pH 2 with 1N aq. HCl, and extracted with 10% MeOH in DCM. The combined organic phase was dried over anhydrous Na₂SO₄, filtered and concentrated under reduced pressure. The product was purified using reverse phase prep-HPLC to afford N-(4-(2-chloro-4-fluorophenyl)-2-oxo-2H-chromen-7-yl)-N-methylglycine (Example 105, 50 mg). LCMS (ESI) Calcd. for C₁₈H₁₃ClFNO₄: 361, found: [M+H]⁺=362. ¹H NMR (400 MHz, DMSO-d₆): δ 7.67 (dd, 1H), 7.51 (dd, 1H), 7.40 (dt, 1H), 6.72 (d, 1H), 6.58-6.53 (m, 2H), 5.96 (s, 1H), 3.92 (s, 2H), 3.02 (s, 3H).

### Example 106: Synthesis of 2-(methyl(2-oxo-4-(o-tolyl)-2H-chromen-7-yl)amino)acetamide

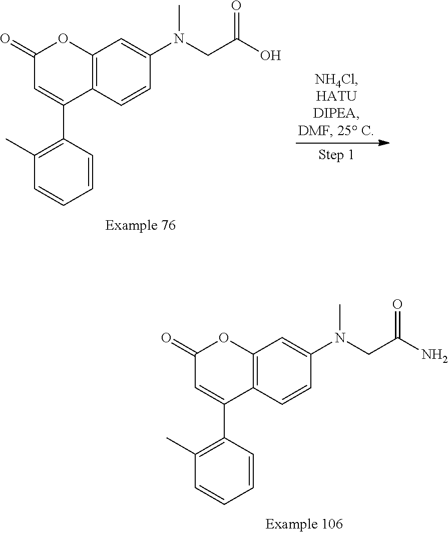 [SMILES: Cc1ccccc1-c1cc(=O)oc2cc(N(C)CC(=O)O)ccc12 ; Cc1ccccc1-c1cc(=O)oc2cc(N(C)CC(N)=O)ccc12]

Synthesis of 2-(methyl(2-oxo-4-(o-tolyl)-2H-chromen-7-yl)amino)acetamide, Example 106 [Step 1]: An oven dried round bottom flask was charged with N-methyl-N-(2-oxo-4-(o-tolyl)-2H-chromen-7-yl)glycine (Example 76, 35 mg, 0.1 mmol) followed by HATU (45 mg, 0.1 mmol), DMF (1 mL), and DIPEA (22 μL, 0.2 mmol). Ammonium Chloride (57 mg, 1.1 mmol) was added to the reaction mixture and stirred at 25° C. for 16 h. The reaction mixture was filtered and partitioned between EtOAc and water. The organic layer was collected, washed with brine, dried over anhydrous Na₂SO₄, and concentrated under reduced pressure. The product was purified using reverse phase prep-HPLC to afford 2-(methyl(2-oxo-4-(o-tolyl)-2H-chromen-7-yl)amino)acetamide (Example 106, 11 mg). LCMS (ESI) Calcd. for C₁₉H₁₈N₂O₃: 322, found: [M+H]⁺=323. ¹H NMR (400 MHz, DMSO-d₆): δ 7.40-7.33 (m, 4H), 7.21-7.19 (m, 1H), 7.14 (s, 1H), 6.73-6.71 (m, 1H) 6.59-5.56 (m, 2H), 5.92 (s, 1H), 4.00 (s, 2H) 3.06 (s, 3H), 2.12 (s, 3H).

### Example 107: Synthesis of 2-(methyl(2-oxo-4-(o-tolyl)-2H-chromen-7-yl)amino)acetamide

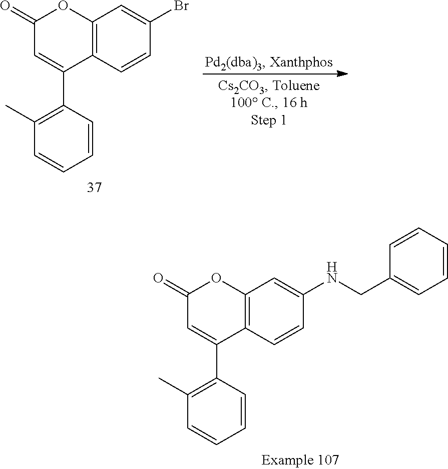 [SMILES: Cc1ccccc1-c1cc(=O)oc2cc(Br)ccc12 ; Cc1ccccc1-c1cc(=O)oc2cc(NCc3ccccc3)ccc12]

Synthesis of 7-(benzylamino)-4-(o-tolyl)-2H-chromen-2-one, Example 107 [Step 1]: To a degassed solution of 7-bromo-4-(o-tolyl) chromen-2-one (37, 100 mg, 0.3 mmol) and benzylamine (0.03 mL, 0.3 mmol) in toluene (2 mL) was added Cs₂CO₃ (207 mg, 0.6 mmol) and Xanthphos (40 mg, 0.06 mmol). The mixture was purged with Argon for 5 min., Pd₂(dba)₃ (30 mg, 0.03 mmol) was added, and the reaction was heated at 100° C. for 16 h. Reaction mixture was filtered through celite bed and washed with ethyl acetate. The organic layer was washed with water and dried over Na₂SO₄, evaporated under reduced pressure and purified by reverse phase prep-HPLC to afford 7-(benzylamino)-4-(o-tolyl) chromen-2-one (Example 107, 24 mg). LCMS (ESI) Calcd. for C₂₃H₁₉NO₂: 341, found [M+H]⁺=342. ¹H NMR (400 MHz, DMSO-d₆) δ 7.41-7.29 (m, 8H), 7.26-7.23 (m, 1H), 7.18 (d, 1H), 6.63 (d, 1H), 6.58-6.56 (m, 1H), 6.46 (d, 1H), 6.85 (s, 1H), 4.36 (d, 2H), 2.09 (s, 3H).

### Example 108: Synthesis of N-methyl-N-(2-oxo-4-(o-tolyl)-2H-chromen-7-yl)-D-alanine

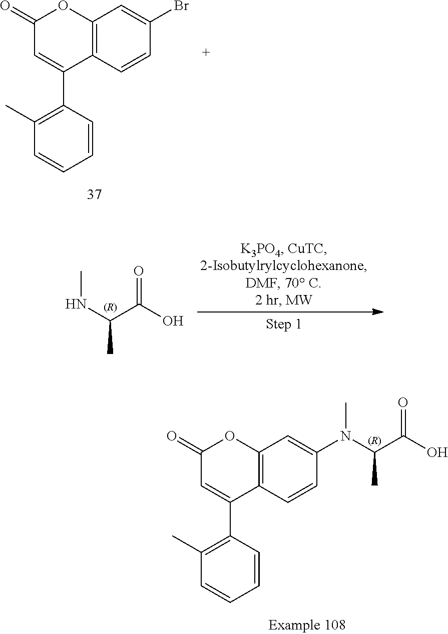

Synthesis of N-methyl-N-(2-oxo-4-(o-tolyl)-2H-chromen-7-yl)-D-alanine, Example 108 [Step 1]: An oven dried microwave tube was charged with 7-bromo-4-(o-tolyl)-2H-chromen-2-one (37, 150 mg, 0.5 mmol) and methyl-D-alanine (59 mg, 0.6 mmol) followed by copper thiophene 2-carboxylate (73 mg, 0.4 mmol), 2-isobutyrylcyclohexanone (16 μL, 0.1 mmol) and DMF (3 mL). Reaction mixture was heated at 70° C. for 2 h. in microwave. The reaction mixture was filtered and partitioned between EtOAc and water. The aqueous layer was acidified to pH 5-6 using 2N HCl and extracted with 10% MeOH in DCM. The combined organic layer washed with ice-cold brine, dried over anhydrous Na₂SO₄, and concentrated under reduced pressure. The product was purified using reverse phase prep-HPLC to afford N-methyl-N-(2-oxo-4-(o-tolyl)-2H-chromen-7-yl)-D-alanine (Example 108, 20 mg). ¹H NMR (400 MHz, DMSO-d₆): δ 7.40-7.31 (m, 3H), 7.22-7.19 (m, 1H), 6.71-6.68 (m, 3H), 5.90 (s, 1H), 4.57 (s, 1H) 2.86 (s, 3H), 2.11 (s, 3H), 1.36-1.34 (m, 3H). LCMS (ESI) Calcd. for C₂₀H₁₉NO₄: 337, found: [M+H]⁺=338.

### Examples 109-114: Synthesis of N-(1-(2-oxo-4-(o-tolyl)-2H-chromene-7-carbonyl)piperidin-4-yl)acetamide, N-methyl-N-(1-(2-oxo-4-(o-tolyl)-2H-chromene-7-carbonyl)piperidin-4-yl)acetamide, N-methyl-2-oxo-N-(tetrahydro-2H-pyran-4-yl)-4-(o-tolyl)-2H-chromene-7-carboxamide, N-(2-amino-2-oxoethyl)-N-methyl-2-oxo-4-(o-tolyl)-2H-chromene-7-carboxamide, N-(2-amino-2-oxoethyl)-2-oxo-4-(o-tolyl)-2H-chromene-7-carboxamide, N-methyl-N-((1-methyl-1H-imidazol-2-yl)methyl)-2-oxo-4-(o-tolyl)-2H-chromene-7-carboxamide

### General Reaction Scheme:

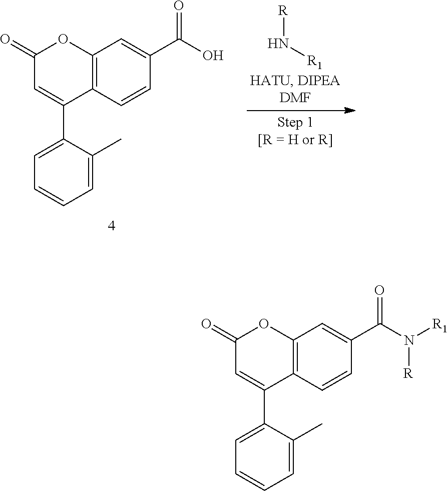

| R—NH₂ | Example |
|---|---|
| 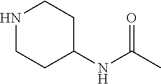 | 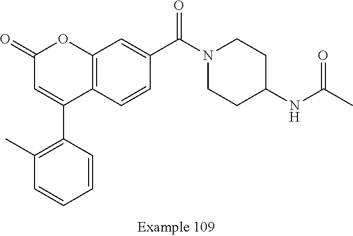 [SMILES: CC(=O)NC1CCN(C(=O)c2ccc3c(-c4ccccc4C)cc(=O)oc3c2)CC1] |
| 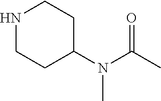 | 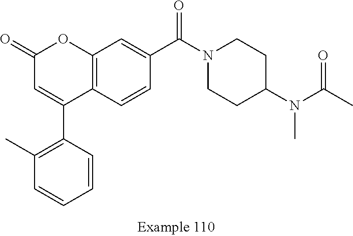 [SMILES: CC(=O)N(C)C1CCN(C(=O)c2ccc3c(-c4ccccc4C)cc(=O)oc3c2)CC1] |
| 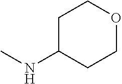 | 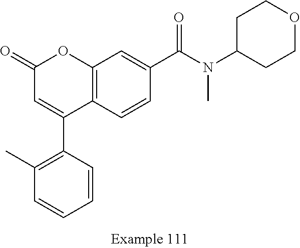 [SMILES: Cc1ccccc1-c1cc(=O)oc2cc(C(=O)N(C)C3CCOCC3)ccc12] |
| 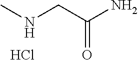 | 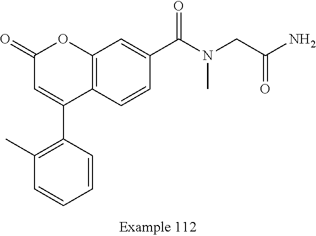 [SMILES: Cc1ccccc1-c1cc(=O)oc2cc(C(=O)N(C)CC(N)=O)ccc12] |
| 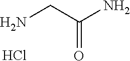 | 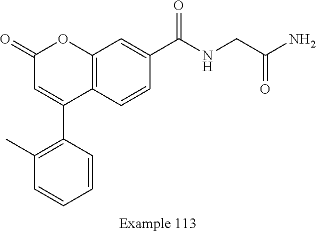 [SMILES: Cc1ccccc1-c1cc(=O)oc2cc(C(=O)NCC(N)=O)ccc12] |
| 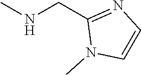 | 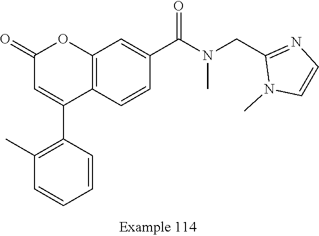 [SMILES: Cc1ccccc1-c1cc(=O)oc2cc(C(=O)N(C)Cc3nccn3C)ccc12] |

General procedure for acid amine coupling [Step 1]: To a solution of HATU (1.1 mmol) in DMF (1.5 mL), 2-oxo-4-(o-tolyl)-2H-chromene-7-carboxylic acid (4, 150 mg, 0.54 mmol) was added followed by N,N-Diisopropylethylamine (1.6 mmol). The reaction mixture was stirred for 15 minutes at ambient temperature under nitrogen balloon. Then the corresponding amine (1.1 mmol) was added to the reaction mixture was the solution was stirred for 16 h. at ambient temperature. Then the reaction mixture was diluted with ethyl acetate and washed with 5% aqueous K₂CO₃ solution. The organic phase was then washed with ice cold water and brine. It was dried over Na₂SO₄, filtered and evaporated to yield the product which was purified through reverse phase prep-HPLC and lyophilized.

N-(1-(2-oxo-4-(o-tolyl)-2H-chromene-7-carbonyl)piperidin-4-yl)acetamide, Example 109: LCMS (ESI) Calcd for C₂₄H₂₄N₂O₄: 404, found: [M+H]⁺=405. ¹H NMR (400 MHz, DMSO-d₆) δ 7.84 (d, 1H), 7.50-7.40 (m, 3H), 7.39-7.34 (m, 1H), 7.30-7.21 (m, 2H), 7.03 (d, 1H), 6.46 (s, 1H), 4.41-4.18 (m, 1H), 3.96-3.69 (m, 1H), 3.58-3.44 (m, 1H), 3.23-2.92 (m, 2H), 2.13 (s, 3H), 1.90-1.79 (m, 1H), 1.79 (s, 3H), 1.75-1.65 (m, 1H), 1.45-1.17 (m, 2H).

N-methyl-N-(1-(2-oxo-4-(o-tolyl)-2H-chromene-7-carbonyl)piperidin-4-yl)acetamide, Example 110: LCMS (ESI) Calcd. for C₂₅H₂₆N₂O₄: 418, found: [M+H]⁺=419. ¹H NMR (400 MHz, DMSO-d₆ at 100° C.) δ 7.49-7.27 (m, 6H), 7.06-7.04 (d, 1H), 6.38 (s, 1H), 4.10 (m, 4H), 3.03-2.95 (m, 1H), 2.79 (s, 3H), 2.16 (s, 3H), 2.01 (s, 3H), 1.74-1.17 (m, 4H).

N-methyl-2-oxo-N-(tetrahydro-2H-pyran-4-yl)-4-(o-tolyl)-2H-chromene-7-carboxamide, Example 111: LCMS (ESI) Calcd. for C₂₃H₂₃NO₄: 377, found: [M+H]⁺=378. ¹H NMR (400 MHz, DMSO-d₆ at 100° C.) δ 7.50-7.33 (m, 4H), 7.26 (dd, 2H), 7.05 (d, 1H), 6.39 (s, 1H), 4.28-3.98 (m, 1H), 3.91 (dd, 2H), 3.29 (t, 2H), 2.84 (s, 3H), 2.16 (s, 3H), 1.93-1.77 (m, 2H), 1.62 (d, 2H).

N-(2-amino-2-oxoethyl)-N-methyl-2-oxo-4-(o-tolyl)-2H-chromene-7-carboxamide, Example 112: LCMS (ESI) Calcd. for C₂₀H₁₈N₂O₄: 350, found: [M+H]⁺=351. ¹H NMR (400 MHz, DMSO-d₆) δ 7.50-7.34 (m, 5H), 7.36-7.22 (m, 2H), 7.16 (d, 1H), 7.03 (dd, 1H), 6.45 (d, 1H), 4.02 (s, 1H), 3.79 (s, 1H), 2.94 (d, 3H), 2.12 (d, 3H).

N-(2-amino-2-oxoethyl)-2-oxo-4-(o-tolyl)-2H-chromene-7-carboxamide, Example 113: LCMS (ESI) Calcd. for C₁₉H₁₆N₂O₄: 336, found: [M+H]⁺=337. ¹H NMR (400 MHz, DMSO-d₆ at 100° C.) δ 8.92 (t, 1H), 7.95 (s, 1H), 7.75 (d, 1H), 7.51-7.33 (m, 4H), 7.29 (d, 1H), 7.07 (d, 2H), 6.49 (s, 1H), 3.83 (d, 2H), 2.11 (s, 3H).

N-methyl-N-((1-methyl-1H-imidazol-2-yl)methyl)-2-oxo-4-(o-tolyl)-2H-chromene-7-carboxamide, Example 114: LCMS (ESI) Calcd. for C₂₃H₂₁N₃O₃: 387, found: [M+H]⁺=388. ¹H NMR (400 MHz, DMSO-d₆) δ 7.72-7.52 (m, 1H), 7.50-7.22 (m, 5H), 7.19-6.95 (m, 2H), 6.85 (d, 1H), 6.46 (s, 1H), 4.72 (s, 1H), 4.50 (s, 1H), 3.67 (s, 2H), 3.54-3.35 (m, 1H), 2.88 (d, 3H), 2.12 (s, 3H).

### Examples 115-118: Synthesis of 7-(3-(methoxymethyl) azetidine-1-carbonyl)-4-(o-tolyl)-2H-chromen-2-one, (R)-7-(3-(methoxymethyl)pyrrolidine-1-carbonyl)-4-(o-tolyl)-2H-chromen-2-one, N-methyl-2-oxo-N-(tetrahydro-2H-pyran-3-yl)-4-(o-tolyl)-2H-chromene-7-carboxamide, 7-(3-(hydroxymethyl) azetidine-1-carbonyl)-4-(o-tolyl)-2H-chromen-2-one

### General Reaction Scheme:

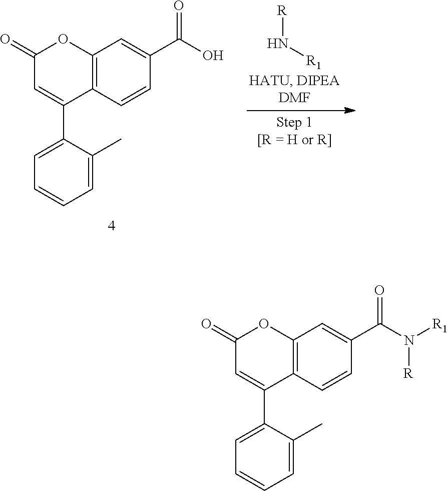

| R—NH₂ | Example |
|---|---|
| 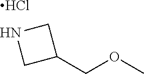 | 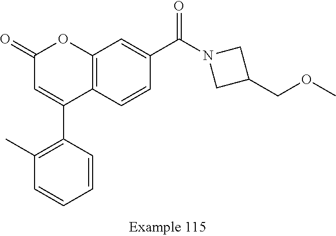 [SMILES: COCC1CN(C(=O)c2ccc3c(-c4ccccc4C)cc(=O)oc3c2)C1] |
| 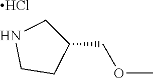 | 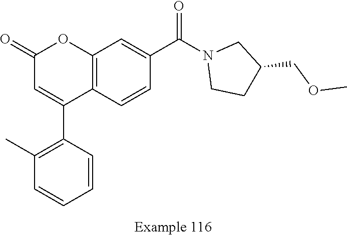 [SMILES: COC[C@@H]1CCN(C(=O)c2ccc3c(-c4ccccc4C)cc(=O)oc3c2)C1] |
| 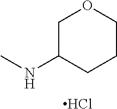 | 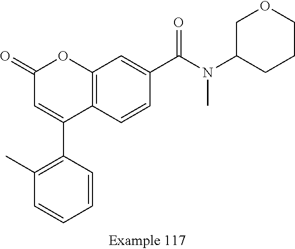 [SMILES: Cc1ccccc1-c1cc(=O)oc2cc(C(=O)N(C)C3CCCOC3)ccc12] |
| 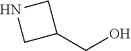 | 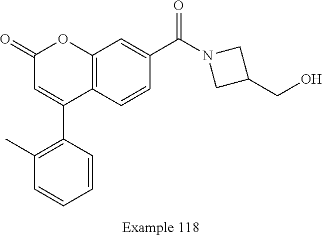 [SMILES: Cc1ccccc1-c1cc(=O)oc2cc(C(=O)N3CC(CO)C3)ccc12] |

General procedure for acid amine coupling [Step 1]: To a solution of HATU (1.1 mmol) in DMF (1.5 mL), 2-oxo-4-(o-tolyl)-2H-chromene-7-carboxylic acid (4, 150 mg, 0.5 mmol) was added followed by N,N-Diisopropylethylamine (1.6 mmol). The reaction mixture was stirred for 15 minutes at ambient temperature under nitrogen balloon. Then corresponding amine (1.1 mmol) was added to the reaction mixture and the solution was stirred for 16 h. at ambient temperature. Then the reaction mixture was diluted with ethyl acetate and washed with 5% aqueous K₂CO₃ solution. The organic phase was then washed with ice cold water and brine and was dried over Na₂SO₄, filtered and evaporated. The product was purified through reverse phase prep-HPLC and lyophilized to afford corresponding product.

7-(3-(methoxymethyl) azetidine-1-carbonyl)-4-(o-tolyl)-2H-chromen-2-one, Example 115: LCMS (ESI) Calcd. for C₂₂H₂₁NO₄: 363, found: [M+H]⁺=364. ¹H NMR (400 MHz, DMSO-d₆) δ 7.66 (br s, 1H), 7.51-7.35 (m, 4H), 7.27 (d, 1H), 7.03 (d, 1H), 6.48 (s, 1H), 4.39-4.35 (m, 1H), 4.12-4.01 (m, 2H), 3.79-3.75 (m, 1H), 3.49 (d, 2H), 3.26 (s, 3H), 2.89-2.82 (m, 1H), 2.12 (s, 3H).

(R)-7-(3-(methoxymethyl)pyrrolidine-1-carbonyl)-4-(o-tolyl)-2H-chromen-2-one, Example 116: LCMS (ESI) Calcd. for C₂₃H₂₃NO₄: 377, found: [M+H]⁺=378. ¹H NMR (400 MHz, DMSO-d₆ at 100° C.) δ 7.54 (br s, 1H), 7.47-7.35 (m, 4H), 7.27 (d, 1H), 7.04 (d, 1H), 6.39 (s, 1H), 3.56 (br s, 2H), 3.49-3.46 (m, 1H), 3.38-3.32 (m, 2H), 3.27 (br s, 4H), 2.49-2.45 (m, 1H), 2.16 (s, 3H), 2.03-1.95 (m, 1H), 1.72-1.63 (m, 1H).

N-methyl-2-oxo-N-(tetrahydro-2H-pyran-3-yl)-4-(o-tolyl)-2H-chromene-7-carboxamide, Example 117: LCMS (ESI) Calcd. for C₂₃H₂₃NO₄: 377, found: [M+H]⁺=378. ¹H NMR (400 MHz, DMSO-d₆ at 100° C.) δ 7.47-7.35 (m, 4H), 7.28 (d, 1H), 7.24 (d, 1H), 7.05 (d, 1H), 6.38 (s, 1H), 3.91 (br s, 1H), 3.78 (dd, 1H), 3.72 (d, 1H), 3.48 (t, 1H), 3.27 (t, 1H), 2.87 (s, 3H), 2.16 (s, 3H), 1.91-1.85 (m, 2H), 1.69 (d, 1H), 1.54 (br s, 1H).

7-(3-(hydroxymethyl) azetidine-1-carbonyl)-4-(o-tolyl)-2H-chromen-2-one, Example 118: LCMS (ESI) Calcd. for C₂₁H₁₉NO₄: 349, found: [M+H]⁺=350. ¹H NMR (400 MHz, DMSO-d₆) δ 7.65 (s, 1H), 7.51-7.35 (m, 4H), 7.27 (d, 1H), 7.04 (d, 1H), 6.48 (s, 1H), 4.79 (t, 1H), 4.37-4.31 (m, 1H), 4.08-4.00 (m, 2H), 3.82-3.78 (m, 1H), 3.53 (t, 2H), 2.74-2.69 (m, 1H), 2.12 (s, 3H).

### Example 119: Synthesis of 7-(((1-methyl-1H-pyrazol-3-yl)methyl)amino)-4-(o-tolyl)-2H-chromen-2-one

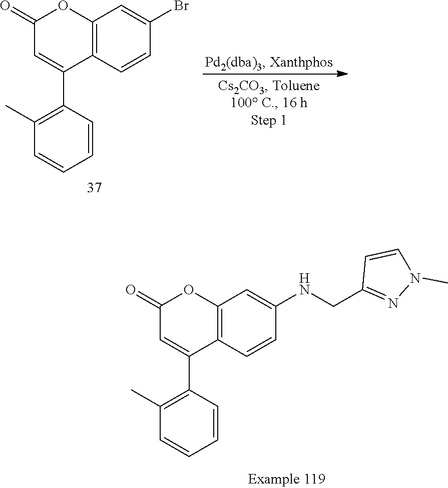 [SMILES: Cc1ccccc1-c1cc(=O)oc2cc(Br)ccc12 ; Cc1ccccc1-c1cc(=O)oc2cc(NCc3ccn(C)n3)ccc12]

Synthesis of 7-(((1-methyl-1H-pyrazol-3-yl)methyl)amino)-4-(o-tolyl)-2H-chromen-2-one, Example 119 [Step 1]: To a degassed solution of 7-bromo-4-(o-tolyl) chromen-2-one (37, 90 mg, 0.3 mmol) and (1-methylpyrazol-3-yl)methanamine (35 mg, 0.3 mmol) in toluene (2 mL) was added Cs₂CO₃ (186 mg, 0.5 mmol) and Xanthphos (33 mg, 0.05 mmol). The mixture was purged with argon for 5 min. and Pd₂(dba)₃ (26 mg, 0.03 mmol) was added and the reaction was heated at 100° C. for 16 h. The reaction mixture was filtered through a celite bed and washed with ethyl acetate. The organic layer was washed with water and dried over Na₂SO₄, evaporated under reduced pressure and purified by reverse phase prep-HPLC to afford 7-(((1-methyl-1H-pyrazol-3-yl)methyl)amino)-4-(o-tolyl)-2H-chromen-2-one (Example 119, 24 mg). ¹H NMR (400 MHz, DMSO-d₆) δ 7.59 (d, 1H), 7.39-7.31 (m, 3H), 7.19-7.15 (m, 2H), 6.63-6.55 (m, 3H), 6.15 (d, 1H), 5.83 (s, 1H), 4.24 (d, 2H), 3.79 (s, 3H), 2.10 (s, 3H). LCMS (ESI) Calcd. for C₂₁H₁₉N₃O₂: 345, found: [M+H]⁺=346.

### Examples 120-121: Synthesis of (S)-1-(N,O-dimethyl-N-(2-oxo-4-(o-tolyl)-2H-chromen-7-yl)-L-seryl)piperidine-3-carboxylic acid, (S)-1-(N,O-dimethyl-N-(2-oxo-4-(o-tolyl)-2H-chromen-7-yl)seryl)piperidine-3-carboxylic acid

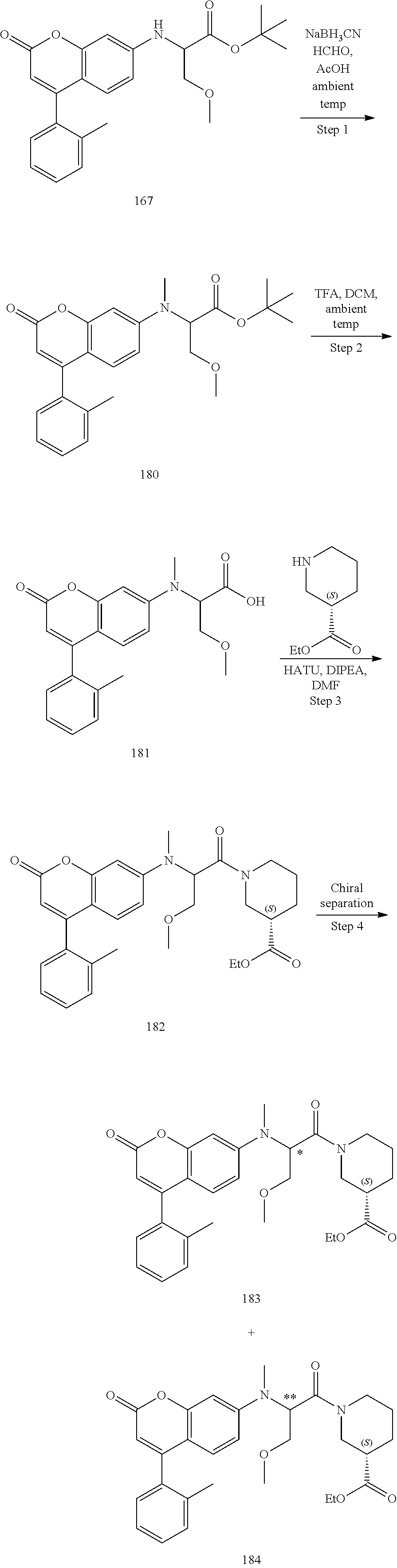

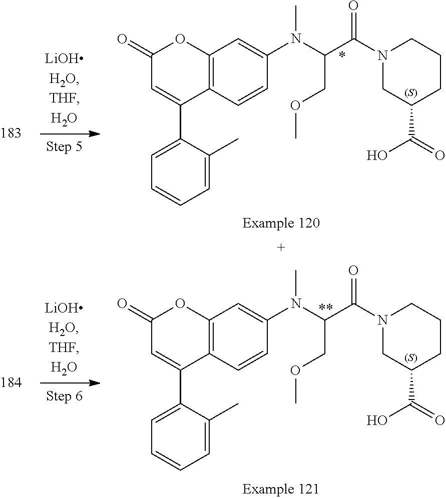

Synthesis of tert-butyl N,O-dimethyl-N-(2-oxo-4-(o-tolyl)-2H-chromen-7-yl) serinate, 180 [Step 1]: An oven dried round bottom flask was charged with tert-butyl O-methyl-N-(2-oxo-4-(o-tolyl)-2H-chromen-7-yl) serinate (167, 400 mg, 1.0 mmol) followed by acetic acid (4 mL), formaldehyde solution (34 μL, 9.74 mmol). Sodium cyanoborohydride (184 mg, 2.9 mmol) was added and stirred at 25° C. for 16 h. The reaction mixture was quenched with water and diluted with MeOH-DCM. The organic layer was collected, washed with brine, dried over anhydrous Na₂SO₄. and concentrated under reduced pressure. The tert-butyl N,O-dimethyl-N-(2-oxo-4-(o-tolyl)-2H-chromen-7-yl) serinate (180, 400 mg) was used directly for the next step. LCMS (ESI) Calcd. For C₂₅H₂₉NO₅: 423, found: [M+H]⁺=424.

N,O-dimethyl-N-(2-oxo-4-(o-tolyl)-2/I-chromen-7-yl) serine, 181 [Step 2]: An oven-dried round bottle flask charged with of tert-butyl N,O-dimethyl-N-(2-oxo-4-(o-tolyl)-2H-chromen-7-yl) serinate (180, 400 mg, 0.6 mmol) DCM (4 mL). Trifluoroacetic acid (0.5 mL, 6.14 mmol) was added to the reaction mixture and flushed with argon and stirred at 25° C. for 6 h. The reaction mixture was filtered and partitioned between DCM and water. The DCM layer was collected and washed with brine, dried over anhydrous Na₂SO₄. and concentrated under reduced pressure. The N,O-dimethyl-N-(2-oxo-4-(o-tolyl)-2H-chromen-7-yl) serine (181, 300 mg) was used directly in next step. LCMS (ESI) Calcd. For C₂₁H₂₁NO₅: 367, found: [M+H]⁺=368.

Synthesis of ethyl (3S)-1-(N,O-dimethyl-N-(2-oxo-4-(o-tolyl)-2H-chromen-7-yl)seryl)piperidine-3-carboxylate, 182 [Step 3]: An oven-dried round bottom flask was charged with ethyl (S)-piperidine-3-carboxylate (181, 0.1 mL, 0.9 mmol) followed by DMF (3 mL), N,O)-dimethyl-N-(2-oxo-4-(o-tolyl)-2H-chromen-7-yl) serine (182, 300 mg, 0.9 mmol). HATU (341 mg, 0.9 mmol) and DIPEA (0.2 mL, 1.2 mmol) were added to it and flushed with argon and stirred at 25° C. for 16 h. The reaction mixture was filtered and partitioned between EtOAc and water. The collected organic layer was washed with brine thrice, dried over anhydrous Na₂SO₄. and concentrated under reduced pressure. This product was purified via flash column chromatography to afford ethyl (3S)-1-(N,O-dimethyl-N-(2-oxo-4-(o-tolyl)-2H-chromen-7-yl)seryl)piperidine-3-carboxylate (182, 210 mg).

Synthesis of diastereomeric ethyl (S)-1-(N,O)-dimethyl-N-(2-oxo-4-(o-tolyl)-2H-chromen-7-yl)seryl)piperidine-3-carboxylate, 183 and 184 [Step 4]: Ethyl (3S)-1-(N,O)-dimethyl-N-(2-oxo-4-(o-tolyl)-2H-chromen-7-yl)seryl)piperidine-3-carboxylate (182, 210 mg) was separated via chiral prep-HPLC purification and lyophilized to afford Peak 1 as ethyl (S)-1-(N,O)-dimethyl-N-(2-oxo-4-(o-tolyl)-2H-chromen-7-yl)seryl)piperidine-3-carboxylate (183, 75 mg) and Peak 2 as ethyl (S)-1-(N,O)-dimethyl-N-(2-oxo-4-(o-tolyl)-2H-chromen-7-yl)seryl)piperidine-3-carboxylate (184, 100 mg). The absolute stereochemistry of these Examples was not determined. 0390 Peak 1: Ethyl(S)-1-(N,O)-dimethyl-N-(2-oxo-4-(o-tolyl)-2H-chromen-7-yl)seryl)piperidine-3-carboxylate, 183: LCMS (ESI) Calcd. For C₂₉H₃₄N₂O₆: 506, found: [M+H]⁺=507.

Peak 2: Ethyl(S)-1-(N,O-dimethyl-N-(2-oxo-4-(o-tolyl)-2H-chromen-7-yl)seryl)piperidine-3-carboxylate, 184: LCMS (ESI) Calcd. For C₂₉H₃₄N₂O₆: 506, found: [M+H]⁺=507.

Chiral prep-HPLC method: Prep-HPLC chiral method: Chiral separation was performed on an Agilent 1200 series instrument. Column was a CHIRALPAK IC (250×20 mm), 5μ, operating at ambient temperature and flow rate of 18.0 mL/min. Mobile phase was a mixture of 70% hexane, 15% dichloromethane and 15% of ethanol, held isocratic for up to 22 min. with detection at 358 nm wavelength.

Synthesis of (S)-1-(N,O)-dimethyl-N-(2-oxo-4-(o-tolyl)-2H-chromen-7-yl)-L-seryl)piperidine-3-carboxylic acid, Example 120 [Step 5]: A round bottom flask was charged with ethyl (S)-1-(N,O-dimethyl-N-(2-oxo-4-(o-tolyl)-2H-chromen-7-yl)seryl)piperidine-3-carboxylate (183, 75 mg, 0.1 mmol) in THF (4.5 mL) and water (0.5 mL). Lithium hydroxide (18 mg, 0.7 mmol) was added to the mixture and stirred at 25° C. for 6 h. Water and DCM were added to the reaction mixture. The aqueous layer was collected and acidified using 1N HCl slowly until pH reached nearly 5-6. The solution was then extracted with 15% MeOH-DCM. The combined organic layer was dried over Na₂SO₄ and concentrated under reduced pressure. The product was purified using reverse phase prep-HPLC to afford(S)-1-(N,O-dimethyl-N-(2-oxo-4-(o-tolyl)-2H-chromen-7-yl)-L-seryl)piperidine-3-carboxylic acid (Example 120, 19 mg). LCMS (ESI) Calcd. for C₂₇H₃₀N₂O₆: 478, found: [M+H]⁺=479. ¹H NMR (400 MHz, DMSO-d₆): δ 7.41-7.33 (m, 3H), 7.23-7.21 (m, 1H), 6.80-6.77 (m, 3H), 5.91 (s, 1H), 5.05 (s, 1H) 4.07-3.67 (m, 4H), 3.52-3.27 (m, 5H), 2.89 (s, 3H), 2.23-2.15 (m, 4H) 1.85-1.67 (m, 1H), 1.57-1.36 (m, 3H) 1.27-1.18 (m, 1H).

Synthesis of (S)-1-(N,O-dimethyl-N-(2-oxo-4-(o-tolyl)-2H-chromen-7-yl)seryl)piperidine-3-carboxylic acid, Example 121 [Step 6]: An oven dried round bottom flask was charged with ethyl (S)-1-(N,O-dimethyl-N-(2-oxo-4-(o-tolyl)-2H-chromen-7-yl)seryl)piperidine-3-carboxylate (184, 100 mg, 0.2 mmol) followed by THF (4.5 mL) and water (0.5 mL). Lithium hydroxide (24 mg, 1.0 mmol) was added to the reaction and stirred at 25° C. for 6 h. Water and DCM was added to the reaction mixture. The aqueous layer was collected and acidified using 1N HCl slowly until pH reached nearly 5-6. The solution was then extracted with 15% MeOH-DCM. The combined organic layer was dried over Na₂SO₄ and concentrated under reduced pressure. The product was purified using reverse phase prep-HPLC to afford(S)-1-(N,O-dimethyl-N-(2-oxo-4-(o-tolyl)-2H-chromen-7-yl)seryl)piperidine-3-carboxylic acid (Example 121, 18 mg). LCMS (ESI) Calcd. for C₂₇H₃₀N₂O₆: 478, found: [M+H]⁺=479. ¹H NMR (400 MHz, DMSO-d₆): δ 7.41-7.32 (m, 3H), 7.23-7.21 (m, 1H), 6.87-6.74 (m, 3H), 5.92 (s, 1H), 5.0 (s, 1H), 3.77-3.69 (m, 2H), 3.31-3.28 (m, 3H), 2.98-2.92 (m, 4H), 2.15 (s, 3H) 1.96-1.93 (m, 1H), 1.58-1.55 (m, 2H), 1.37-1.27 (m, 2H), 1.22-1.18 (m, 3H), one peak merged with solvent peak.

### Examples 122-123: Synthesis of N-((1-methyl-1H-imidazol-2-yl)methyl)-2-oxo-4-(o-tolyl)-2H-chromene-7-carboxamide, (S)-7-(3-(methoxymethyl)pyrrolidine-1-carbonyl)-4-(o-tolyl)-2H-chromen-2-one

### General Reaction Scheme:

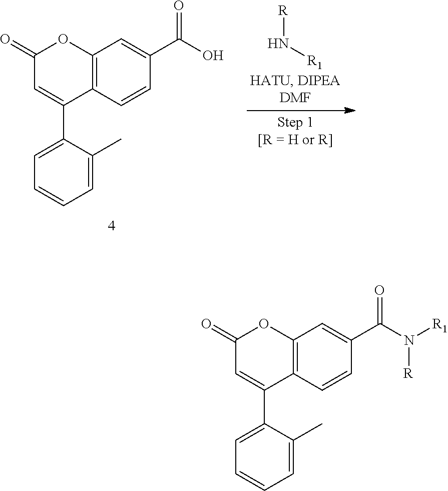

| RNH₂ | Structure & ID |
|---|---|
| 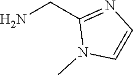 | 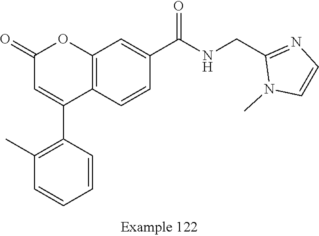 [SMILES: Cc1ccccc1-c1cc(=O)oc2cc(C(=O)NCc3nccn3C)ccc12] |
| 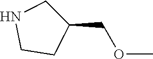 | 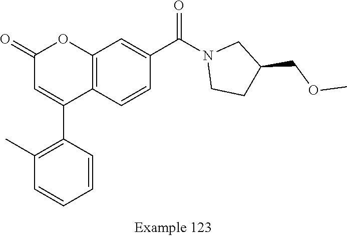 [SMILES: COC[C@H]1CCN(C(=O)c2ccc3c(-c4ccccc4C)cc(=O)oc3c2)C1] |

General procedure for acid amine coupling [Step 1]: To a solution of HATU (1.07 mmol) in DMF (1.5 mL), 2-oxo-4-(o-tolyl)-2H-chromene-7-carboxylic acid (4, 150 mg, 0.535 mmol) were added followed by N,N-Diisopropylethylamine (1.61 mmol). The reaction mixture was stirred for 15 minutes at ambient temperature under a nitrogen balloon. Then corresponding amine (1.07 mmol) was added to the reaction mixture and stirred the solution for 16 h. at ambient temperature. Then the reaction mixture was diluted with ethyl acetate and washed with 5% aqueous K₂CO₃ solution. The organic phase was then washed with ice cold water and brine. The mixture was dried over Na₂SO₄, filtered and evaporated to yield the product was, which purified through reverse phase prep-HPLC and lyophilized.

N-((1-methyl-1H-imidazol-2-yl)methyl)-2-oxo-4-(o-tolyl)-2H-chromene-7-carboxamide, Example 122: LCMS (ESI) Calcd. for C₂₂H₁₉N₃O₃: 373, found: [M+H]⁺=374. ¹H NMR (400 MHz, DMSO-d₆) δ 9.20 (m, 1H), 7.95 (s, 1H), 7.77-7.75 (d, 1H), 7.47-7.35 (m, 3H), 7.29-7.27 (d, 1H), 7.08-7.04 (m, 2H), 6.80 (s, 1H), 6.48 (s, 1H), 4.54 (d, 2H), 3.64 (s, 3H), 2.11 (s, 3H).

(S)-7-(3-(methoxymethyl)pyrrolidine-1-carbonyl)-4-(o-tolyl)-2H-chromen-2-one, Example 123: LCMS (ESI) Calcd. for C₂₃H₂₃NO₄: 377, found: [M+H]⁺=378. ¹H NMR (400 MHz, DMSO-d₆) δ 7.60 (d, 1H), 7.47-7.35 (m, 4H), 7.29-7.27 (d, 1H), 7.03-7.00 (dd, 1H), 6.46 (s, 1H), 3.62-3.55 (m, 1H), 3.49-3.41 (m, 2H), 3.39-3.34 (m, 1H), 3.30-3.15 (m, 5H), 2.50-2.40 (m, 1H), 2.13 (s, 3H), 1.99-1.90 (m, 1H), 1.66-1.61 (m, 1H).

### Example 124: Synthesis of 7-(3-(hydroxymethyl)-3-methylazetidine-1-carbonyl)-4-(o-tolyl)-2H-chromen-2-one

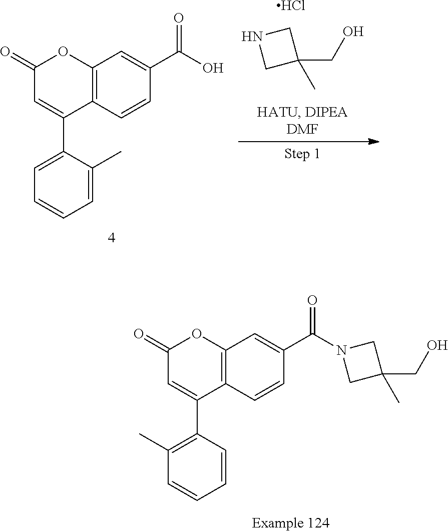

Synthesis of 7-(3-(hydroxymethyl)-3-methylazetidine-1-carbonyl)-4-(o-tolyl)-2H-chromen-2-one, Example 124 [Step 1]: To a stirred solution of 2-oxo-4-(o-tolyl)-2H-chromene-7-carboxylic acid (4, 150 mg, 0.5 mmol) in DMF (2 mL), HATU (305 mg, 0.8 mmol) and N,N-Diisopropylethylamine (0.3 mL, 1.6 mmol) were added at ice cold condition and the reaction mixture was stirred for 30 min. Then (3-methylazetidin-3-yl)methanol hydrochloride (110 mg, 1.1 mmol) was added and the reaction mixture was stirred for 16 h. TLC and LCMS showed formation of desired product. The reaction mixture was filtered through celite bed, and the filtrate was submitted for reverse phase prep-HPLC purification. The fraction obtained was lyophilized to yield 7-(3-(hydroxymethyl)-3-methylazetidine-1-carbonyl)-4-(o-tolyl)-2H-chromen-2-one (Example 124, 85 mg). LCMS (ESI) Calcd. for C₂₂H₂₁NO₄: 363, found: [M+H]⁺=364. ¹H NMR (400 MHz, DMSO-d₆) δ 7.66 (s, 1H), 7.51-7.35 (m, 4H), 7.27 (d, 1H), 7.04 (d, 1H), 6.48 (s, 1H), 4.95 (t, 1H), 4.09 (q, 1H), 3.94-3.89 (m, 2H), 3.10 (d, 1H), 3.35 (d, 2H), 2.12 (s, 3H), 1.19 (s, 3H).

### Example 125: Synthesis of 7-(methyl((1-methyl-1H-pyrazol-3-yl)methyl)amino)-4-(o-tolyl)-2H-chromen-2-one

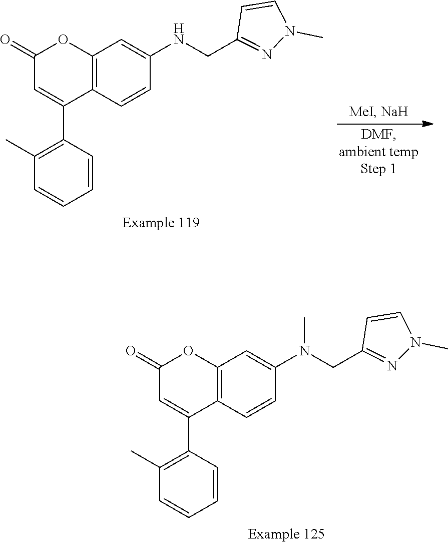 [SMILES: Cc1ccccc1-c1cc(=O)oc2cc(NCc3ccn(C)n3)ccc12 ; Cc1ccccc1-c1cc(=O)oc2cc(N(C)Cc3ccn(C)n3)ccc12]

Synthesis of 7-(methyl((1-methyl-1H-pyrazol-3-yl)methyl)amino)-4-(o-tolyl)-2H-chromen-2-one, Example 125 [Step 1]: To a stirred solution of 7-(((1-methyl-1H-pyrazol-3-yl)methyl)amino)-4-(o-tolyl)-2H-chromen-2-one (Example 119, 250 mg, 0.75 mmol) in dry DMF (5 mL) was added NaH (70 mg, 1.8 mmol, 60% in oil) at 0° C. and stirred for 30 min. at ambient temperature. After that Mel (0.11 mL, 1.8 mmol) was added and stirred at ambient temperature for 16 h. The reaction mixture was quenched with ice-water and extracted with EtOAc and dried over anhydrous sodium sulfate and concentrated under reduced pressure. The product was purified by prep-HPLC and lyophilized to afford 7-(methyl((1-methyl-1H-pyrazol-3-yl)methyl)amino)-4-(o-tolyl)-2H-chromen-2-one (Example 125, 17 mg). LCMS Calcd. for C₂₂H₂₁N₃O₂: 359, found: [M+H]⁺=360. ¹H NMR (400 MHz, DMSO-d₆): δ 7.57-7.56 (d, 1H), 7.40-7.32 (m, 3H), 7.20-7.18 (d, 1H), 6.75-6.68 (m, 3H), 6.08-6.07 (d, 1H), 5.89 (s, 1H), 4.53 (s, 2H), 3.76 (s, 3H), 3.07 (s, 3H), 2.10 (s, 3H).

### Example 126: Synthesis of 7-(benzylamino)-4-(o-tolyl)-2H-chromen-2-one

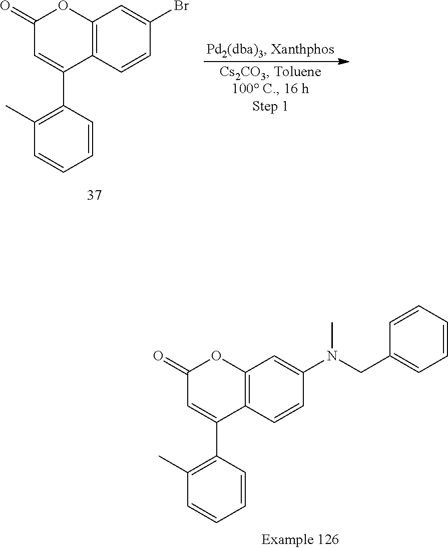 [SMILES: Cc1ccccc1-c1cc(=O)oc2cc(Br)ccc12 ; Cc1ccccc1-c1cc(=O)oc2cc(N(C)Cc3ccccc3)ccc12]

Synthesis of 7-(benzylamino)-4-(o-tolyl)-2H-chromen-2-one, Example 126 [Step 1]: To a degassed solution of 7-bromo-4-(o-tolyl) chromen-2-one (37, 200 mg, 0.6 mmol) and N-methyl-1-phenyl-methanamine (76 mg, 0.6 mmol) in toluene (2 mL) was added Cs₂CO₃ (415 mg, 1.2 mmol) and Xanthphos (75 mg, 0.1 mmol). The mixture was purged with argon for 5 min. and Pd₂(dba)₃ (58 mg, 0.06 mmol) was added, and the reaction was heated at 100° C. for 16 h. The reaction mixture was filtered through celite bed and washed with ethyl acetate. The organic layer was washed with water and dried over Na₂SO₄, evaporated under reduced pressure and purified by reverse phase prep-HPLC to afford 7-[benzyl(methyl)amino]-4-(o-tolyl) chromen-2-one (Example 126, 99 mg). LCMS (ESI): Calcd. for C₂₄H₂₁NO₂: 355, found, [M+H]⁺=356, ¹H NMR (400 MHz, DMSO-d₆) δ 7.41-7.29 (m, 5H), 7.25-7.19 (m, 4H), 6.72 (s, 2H), 6.46 (d, 1H), 5.89 (s, 1H), 4.69 (d, 2H), 3.13 (s, 3H), 2.10 (s, 3H).

### Examples 127-128: Synthesis of 2,2-dimethyl-3-((2-oxo-4-(o-tolyl)-2H-chromen-7-yl)amino)propanoic acid, 2,2-dimethyl-3-(methyl(2-oxo-4-(o-tolyl)-2H-chromen-7-yl)amino)propanoic acid

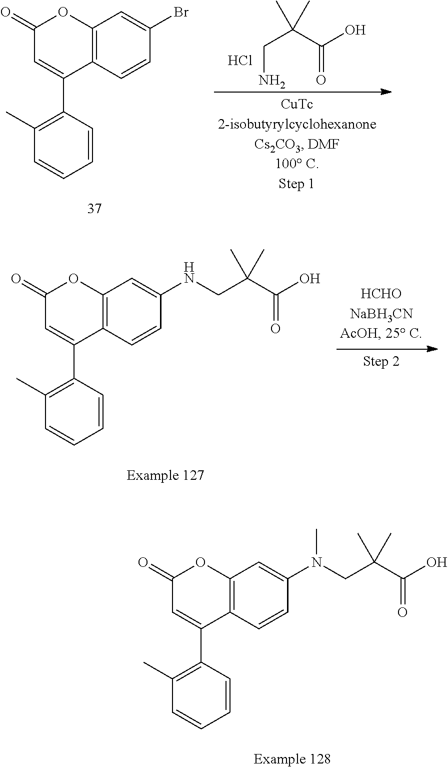

Synthesis of 2,2-dimethyl-3-((2-oxo-4-(o-tolyl)-2H-chromen-7-yl)amino)propanoic acid, Example 127 [Step 1]: A mixture of 3-amino-2,2-dimethylpropanoic acid hydrochloride (117 mg, 0.8 mmol), 7-bromo-4-(o-tolyl)-2H-chromen-2-one (37, 200 mg, 0.6 mmol), copper thiophene 2-carboxylate (24 mg, 0.1 mmol), cesium carbonate (620 mg, 1.9 mmol), 2-isobutyrylcyclohexanone (0.1 mL, 0.5 mmol) and DMF (3 mL) were taken in a sealed tube and was heated to 100° C. for 12 h. The reaction mixture was quenched with water and diluted with ethyl acetate. Water layer was collected, acidified up to pH 2-3 using 2N HCl and extracted using 15% MeOH in dichloromethane. Combined organic extracts were washed with brine, dried over anhydrous Na₂SO₄, filtered and concentrated under reduced pressure. The product was purified by prep-HPLC to afford 2,2-dimethyl-3-((2-oxo-4-(o-tolyl)-2H-chromen-7-yl)amino)propanoic acid (Example 127, 24 mg). LCMS (ESI) Calcd. for C₂₁H₂₁NO₄: 351, found [M+H]⁺=352. ¹H NMR (400 MHz, DMSO-d₆) δ 7.41-7.30 (m, 3H), 7.20-7.18 (m, 1H), 6.74-6.73 (m, 1H) 6.61 (s, 3H), 5.82 (s, 1H), 3.23-3.22 (m, 2H) 2.07 (s, 3H), 1.14 (s, 6H).

Synthesis of 2,2-dimethyl-3-(methyl(2-oxo-4-(o-tolyl)-2H-chromen-7-yl)amino)propanoic acid, Example 128 [Step 2]: Sodium cyanoborohydride (80 mg, 1.28 mmol) was added to a mixture of 2,2-dimethyl-3-((2-oxo-4-(o-tolyl)-2H-chromen-7-yl)amino)propanoic acid (Example 127, 150 mg, 0.4 mmol), formaldehyde, 37% in aqueous solution (0.1 mL, 4.3 mmol) and acetic acid (2 mL) at 0° C. Temperature of the reaction mixture was warmed up to ambient temperature and stirred for 1 h. Reaction mixture was concentrated under reduced pressure. The product was purified using Prep-HPLC to afford 2,2-dimethyl-3-(methyl(2-oxo-4-(o-tolyl)-2H-chromen-7-yl)amino)propanoic acid (Example 128, 29 mg). LCMS (ESI) Calcd. for C₂₂H₂₃NO₄: 365, found [M+H]⁺=366.2. ¹H NMR (400 MHz, DMSO-d₆): δ 12.48 (br s, 1H), 7.43-7.31 (m, 3H), 7.22-7.20 (m, 1H), 6.74-6.68 (m, 3H) 5.91 (s, 1H), 3.69-3.60 (m, 2H) 2.98 (s, 3H), 2.11 (s, 3H), 1.12 (s, 6H).

### Examples 129-130: Synthesis of 3-((4-(2-chloro-4-fluorophenyl)-2-oxo-2H-chromen-7-yl)amino)propanoic acid, 3-((4-(2-chloro-4-fluorophenyl)-2-oxo-2H-chromen-7-yl)(methyl)amino)propanoic acid

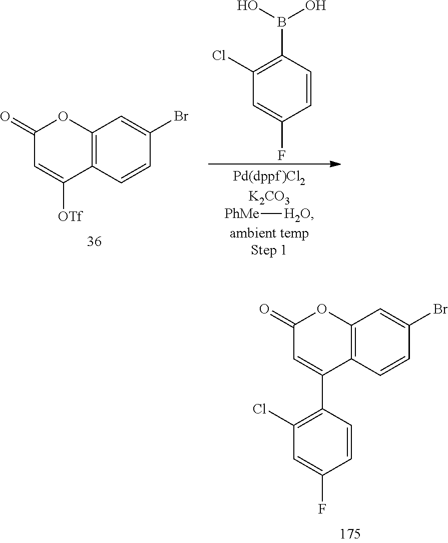

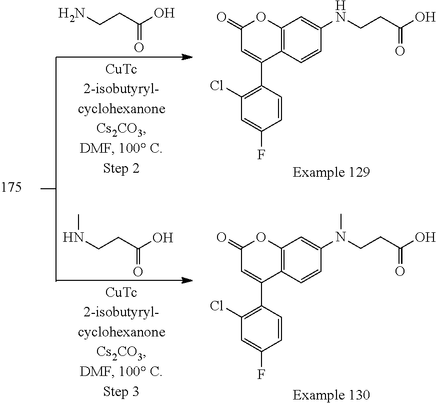

Synthesis of 7-bromo-4-(2-chloro-4-fluorophenyl)-2H-chromen-2-one, 175 [Step 1]: A mixture of 7-bromo-2-oxo-2H-chromen-4-yl trifluoromethanesulfonate (36, 1.00 g, 2.7 mmol), (2-chloro-4-fluorophenyl) boronic acid (2, 421 mg, 2.4 mmol) and potassium carbonate (815 mg, 5.9 mmol) in toluene (10 mL)-water (3 mL) was degassed with argon for 10 minutes. To this solution was added PdCl₂ (dppf) (196 mg, 0.3 mmol) and the reaction mixture was stirred at ambient temperature. After 1 h., the reaction mixture was quenched with water and extracted using ethyl acetate. The combined organic extract was washed with water, brine, dried over anhydrous Na₂SO₄, filtered, and concentrated under reduced pressure. The product was purified using column chromatography to afford 7-bromo-4-(2-chloro-4-fluorophenyl)-2H-chromen-2-one (175, 250 mg). C₁₅H₇BrClFO₂: ¹H NMR (400 MHz DMSO-d₆) δ 7.84 (s, 1H), 7.74-7.72 (m, 1H) 7.60-7.57 (m, 1H), 7.52-7.44 (m, 2H), 6.98 (d, 1H), 6.59 (s, 1H).

Synthesis of 3-((4-(2-chloro-4-fluorophenyl)-2-oxo-2H-chromen-7-yl)amino)propanoic acid, Example 129 [Step 2]: A mixture of 3-aminopropanoic acid (45 mg, 0.5 mmol), 7-bromo-4-(2-chloro-4-fluorophenyl)-2H-chromen-2-one (175, 120 mg, 0.3 mmol), copper thiophene 2-carboxylate (13 mg, 68 μmol), cesium carbonate (221 mg, 0.7 mmol), 2-isobutyrylcyclohexanone (45 μL, 0.3 mmol) and DMF (3 mL) was taken in a sealed tube and heated to 100° C. for 12 h. After cooling, the reaction mixture was quenched with water and ethyl acetate. Water layer was collected, acidified up to pH 2-3 using 2N HCl and extracted with 15% MeOH-DCM. The collected organic layer was washed with brine, dried over anhydrous Na₂SO₄, filtered and concentrated under reduced pressure. The product was purified by prep-HPLC to afford 3-((4-(2-chloro-4-fluorophenyl)-2-oxo-2H-chromen-7-yl)amino)propanoic acid (Example 129, 29 mg). LCMS (ESI) Calcd. for C₁₈H₁₃ClFNO₄: 361, found [M+H]⁺=362. ¹H NMR (400 MHz, methanol-d₄): δ 7.43-7.39 (m, 2H), 7.27-7.23 (m, 1H), 6.78-6.76 (m, 1H), 6.55-6.54 (m, 2H), 5.91 (s, 1H), 3.46-3.43 (m, 2H) 2.57-2.55 (m, 2H), (acidic proton was merged with solvent peak).

Synthesis of 3-((4-(2-chloro-4-fluorophenyl)-2-oxo-2H-chromen-7-yl)(methyl)amino)propanoic acid, Example 130 [Step 3]: A mixture of 3-(methylamino)propanoic acid (52 mg, 0.5 mmol), 7-bromo-4-(2-chloro-4-fluorophenyl)-2H-chromen-2-one (175, 120 mg, 0.3 mmol), Copper thiophene 2-carboxylate (13 mg, 68 μmol), cesium carbonate (221 mg, 0.68 mmol), 2-isobutyrylcyclohexanone (45 μL, 0.3 mmol) and DMF (3 mL) were taken in a sealed and heated to 100° C. for 12 h. After cooling, the reaction mixture was quenched with water and ethyl acetate. Aqueous layer was acidified up to pH 2-3 using 2N HCl and extracted with 15% MeOH-DCM. The collected organic layer was washed with brine, dried over anhydrous Na₂SO₄, filtered and concentrated under reduced pressure. The product was purified by Prep-HPLC to afford 3-((4-(2-chloro-4-fluorophenyl)-2-oxo-2H-chromen-7-yl)(methyl)amino)propanoic acid (Example 130, 24 mg). LCMS (ESI) Calcd. for C₁₉H₁₅ClFNO₄: 375, found [M+H]⁺=376. ¹H NMR (400 MHz, methanol-d₄) δ 6.64-6.60 (m, 2H), 6.48-6.44 (m, 1H), 6.09-6.07 (m, 1H), 5.92-5.87 (m, 2H), 5.16 (s, 1H), 2.98-2.94 (m, 2H), 2.27 (s, 3H), 1.77-1.74 (m, 2H).

### Examples 131-132: Synthesis of 3-((4-(2-chlorophenyl)-2-oxo-2H-chromen-7-yl)amino)propanoic acid, 3-((4-(2-chlorophenyl)-2-oxo-2H-chromen-7-yl)(methyl)amino)propanoic acid

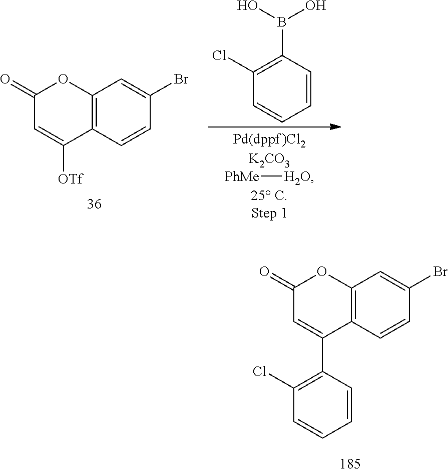

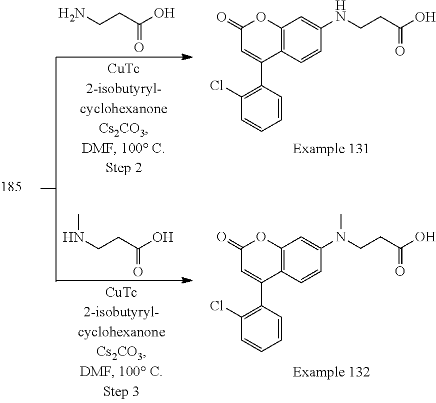

Synthesis of 7-bromo-4-(2-chlorophenyl)-2H-chromen-2-one, 185 [Step 1]: A mixture of 7-bromo-2-oxo-2H-chromen-4-yl trifluoromethanesulfonate (36, 1.00 g, 2.7 mmol), (2-chlorophenyl) boronic acid (377 mg, 2.4 mmol) and potassium carbonate (815 mg, 5.9 mmol) in toluene (10 mL)-water (3 mL) was degassed by bubbling argon for 10 min. Pd (dppf) Cl₂ (196 mg, 0.3 mmol) was added, and the reaction mixture was stirred at ambient temperature. After 1 h., the reaction mixture was diluted with water, and extracted using ethyl acetate. The combined organic extract was washed with brine, dried over anhydrous Na₂SO₄, filtered and concentrated under reduced pressure. The product was purified by column chromatography to afford 7-bromo-4-(2-chlorophenyl)-2H-chromen-2-one (185, 220 mg). LCMS (ESI) Calcd. for C₁₅H₈BrClO₂: 334, found [M+H]⁺=335.

Synthesis of 3-((4-(2-chlorophenyl)-2-oxo-2H-chromen-7-yl)amino)propanoic acid, Example 131 [Step 2]: A sealed tube was charged with 7-bromo-4-(2-chlorophenyl)-2H-chromen-2-one (185, 110 mg, 0.3 mmol), 3-aminopropanoic acid (44 mg, 0.5 mmol), copper thiophene 2-carboxylate (13 mg, 0.1 mmol), cesium carbonate (214 mg, 0.7 mmol), 2-isobutyrylcyclohexanone (40 μL, 0.3 mmol) and DMF (3 mL). The reaction vessel was flushed with argon and capped tightly. The sealed tube was heated in a heating block at 100° C. for 12 hours. After cooling to ambient temperature, the reaction mixture was acidified with 1N aqueous HCl until pH 2 and extracted with 20% methanol-dichloromethane. Combined organic extracts was collected, dried over anhydrous sodium sulfate, filtered and concentrated under reduced pressure. The product was purified by prep-HPLC to afford 3-((4-(2-chlorophenyl)-2-oxo-2H-chromen-7-yl)amino)propanoic acid (Example 131, 30 mg). LCMS (ESI) Calcd. for C₁₈H₁₄ClNO₄: 343, found [M−H]⁺=342. ¹H NMR (400 MHz, DMSO-d₆): δ 7.65-7.63 (m, 1H), 7.56-7.48 (m, 2H), 7.45-7.42 (m, 1H), 6.88 (br s, 1H), 6.66-6.64 (m, 1H), 6.54-6.49 (m, 2H), 5.91 (s, 1H), 3.47-3.22 (t, 2H), 2.45-2.33 (t, 2H).

Synthesis of 3-((4-(2-chlorophenyl)-2-oxo-2H-chromen-7-yl)(methyl)amino)propanoic acid, Example 132 [Step 3]: A sealed tube was charged with 7-bromo-4-(2-chlorophenyl)-2H-chromen-2-one (185, 110 mg, 0.3 mmol), 3-(methylamino)propanoic acid (51 mg, 0.5 mmol), copper thiophene 2-carboxylate (13 mg, 0.1 mmol), cesium carbonate (214 mg, 0.7 mmol), 2-isobutyrylcyclohexanone (40 μL, 0.3 mmol) and DMF (3 mL). The reaction vessel was flushed with argon, and capped tightly. The sealed tube was heated in a heating block at 100° C. for 12 hours. After cooling to ambient temperature, the reaction mixture was acidified with 1N aqueous HCl until pH 2 and extracted with 20% methanol-dichloromethane. The combined organic extract was washed with brine, dried over anhydrous sodium sulfate, filtered and concentrated under reduced pressure. The compound was purified by reverse phase prep-HPLC to afford 3-((4-(2-chlorophenyl)-2-oxo-2H-chromen-7-yl)(methyl)amino)propanoic acid (Example 132, 30 mg). LCMS (ESI) Calcd. for C₁₉H₁₆ClNO₄: 357, found [M+H]⁺=358. ¹H NMR (400 MHz, DMSO-d₆): δ 7.66-7.64 (m, 1H), 7.57-7.49 (m, 2H), 7.46-7.44 (m, 1H), 6.75-6.73 (m, 1H), 6.69-6.66 (m, 2H), 5.98 (s, 1H), 3.67-3.63 (t, 2H), 2.98 (s, 3H), 2.45-2.41 (t, 2H).

### Example 133: Synthesis of 3-(methyl(2-oxo-4-(o-tolyl)-2H-chromen-7-yl)amino)propenamide

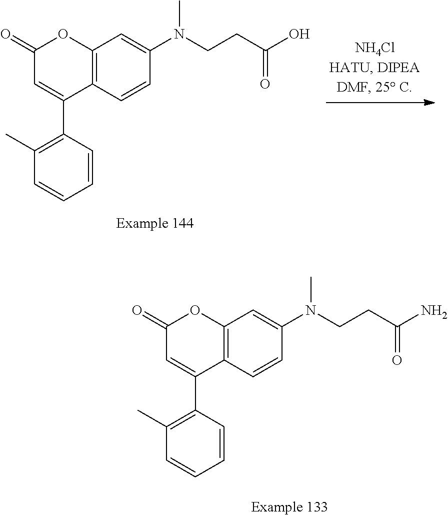 [SMILES: Cc1ccccc1-c1cc(=O)oc2cc(N(C)CCC(=O)O)ccc12 ; Cc1ccccc1-c1cc(=O)oc2cc(N(C)CCC(N)=O)ccc12]

Synthesis of 3-(methyl(2-oxo-4-(o-tolyl)-2H-chromen-7-yl)amino)propenamide, Example 133: To a stirred solution of 3-(methyl(2-oxo-4-(o-tolyl)-2H-chromen-7-yl)amino)propanoic acid (Example 144, 250 mg, 0.7 mmol) and ammonium chloride (589 mg, 11.1 mmol) in dry DMF (3 mL), HATU (465 mg, 1.2 mmol) and DIPEA (0.23 mL, 1.7 mmol) were added. After stirring for 12 h. at ambient temperature, the reaction mixture was quenched with water, and extracted with ethyl acetate. The combined organic extract was washed with brine, dried over anhydrous Na₂SO₄, filtered and concentrated under reduced pressure. The product was purified by reverse phase prep-HPLC to afford 3-(methyl(2-oxo-4-(o-tolyl)-2H-chromen-7-yl)amino)propanamide (Example 133, 80 mg). LCMS (ESI) Calcd. for C₂₀H₂₀N₂O₃: 336, found [M+H]⁺=337.2. ¹H NMR (400 MHz, DMSO-d₆): δ 7.43-7.31 (m, 4H), 7.22-7.20 (m, 1H), 6.88 (s, 1H), 6.73-6.65 (m, 3H), 5.90 (s, 1H), 3.64 (t, 2H) 2.98 (s, 3H), 2.32 (t, 2H), 2.07 (s, 3H).

### Example 134: Synthesis of N-(2-amino-2-oxoethyl)-2-(methyl(2-oxo-4-(o-tolyl)-2H-chromen-7-yl)amino)acetamide

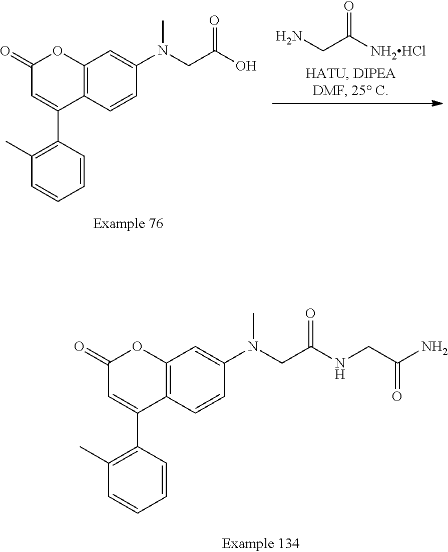

Synthesis of N-(2-amino-2-oxoethyl)-2-(methyl(2-oxo-4-(o-tolyl)-2H-chromen-7-yl)amino)acetamide, Example 134: To a stirred solution of N-methyl-N-(2-oxo-4-(o-tolyl)-2H-chromen-7-yl)glycine (Example 76, 100 mg, 0.3 mmol) and 2-aminoacetamide hydrochloride (2, 50 mg, 0.5 mmol) in DMF (2 mL), HATU (175 mg, 0.5 mmol) and DIPEA (0.1 mL, 0.6 mmol) were added, and the reaction mixture was stirred at 25° C. After 16 h., the reaction mixture was quenched with cold water, and extracted with ethyl acetate. The combined organic extract was washed with brine, dried over anhydrous Na₂SO₄, filtered and concentrated under reduced pressure. The compound was purified by reverse phase prep-HPLC to afford N-(2-amino-2-oxoethyl)-2-(methyl(2-oxo-4-(o-tolyl)-2H-chromen-7-yl)amino)acetamide (Example 134, 65 mg). LCMS (ESI) Calcd. for C₂₁H₂₁N₃O₄: 379; [M+H]⁺=380. ¹H NMR (400 MHz, DMSO-d₆) δ 8.18-8.15 (m, 1H), 7.43-7.31 (m, 4H), 7.21-7.19 (m, 1H), 7.06 (s, 1H), 6.72-6.69 (m, 1H), 6.64-6.60 (m, 2H), 5.92 (s, 1H), 4.12 (s, 2H), 3.64 (d, 2H), 3.06 (s, 3H), 2.11 (s, 3H).

### Examples 135-136: Synthesis of chiral-N-methyl-2-(2-oxo-4-(o-tolyl)-2H-chromen-7-yl)cyclopropane-1-carboxamide

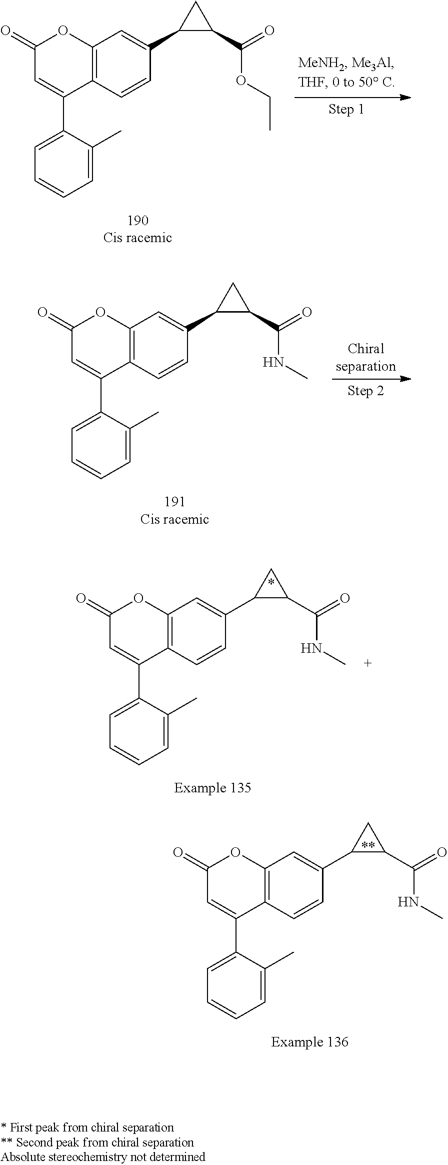 [SMILES: CCOC(=O)[C@@H]1C[C@@H]1c1ccc2c(-c3ccccc3C)cc(=O)oc2c1 ; CNC(=O)[C@@H]1C[C@@H]1c1ccc2c(-c3ccccc3C)cc(=O)oc2c1 ; CNC(=O)C1CC1c1ccc2c(-c3ccccc3C)cc(=O)oc2c1 ; CNC(=O)C1CC1c1ccc2c(-c3ccccc3C)cc(=O)oc2c1]

Synthesis of (cis-rac)-N,N-dimethyl-2-(2-oxo-4-(o-tolyl)-2H-chromen-7-yl)cyclopropane-1-carboxamide, 191 [Step 1]: A stirred solution of ethyl (cis-rac)-2-(2-oxo-4-(o-tolyl)-2H-chromen-7-yl)cyclopropane-1-carboxylate (190, 250 mg, 0.7 mmol) and methyl amine in THF (2.0 mL, 3.6 mmol, 2 M) in toluene (2 mL) and was cooled to 0° C. Trimethylaluminum solution in toluene (0.8 mL, 1.44 mmol, 2 M) was added dropwise at same temperature and reaction mixture was heated at 50° C. for 12 h. After completion, the reaction mixture was quenched with ice water and extracted with ethyl acetate. The combined organic extract was washed with brine solution, dried over anhydrous Na₂SO₄ and concentrated under reduce pressure. The product was purified by reverse phase prep-HPLC and lyophilized to give (cis-rac)-N-methyl-2-(2-oxo-4-(o-tolyl)-2H-chromen-7-yl)cyclopropane-1-carboxamide (191, 120 mg). LCMS (ESI) Calcd. for C₂₂H₂₁NO₃: 347, found [M+H]⁺=348. ¹H NMR (400 MHz, DMSO-d₆) δ 7.98 (t, 1H), 7.46-7.33 (m, 3H), 7.28-7.23 (m, 2H), 7.15 (d, 1H), 6.82 (d, 1H), 6.30 (s, 1H), 2.47-2.42 (m, 4H), 2.11 (s, 3H), 2.08-2.02 (m, 1H), 1.57-1.52 (m, 1H), 1.29-1.24 (m, 1H).

Synthesis of chiral-N-methyl-2-(2-oxo-4-(o-tolyl)-2H-chromen-7-yl)cyclopropane-1-carboxamide, Example 135 and Example 136 [Step 2]: (cis-rac)-N-methyl-2-(2-oxo-4-(o-tolyl)-2H-chromen-7-yl)cyclopropane-1-carboxamide (191, 120 mg, 0.4 mmol) was submitted for SFC chiral separation and the fractions obtained were lyophilized to get the first product as chiral-N-methyl-2-(2-oxo-4-(o-tolyl)-2H-chromen-7-yl)cyclopropane-1-carboxamide (Example 135, Peak 1, 35 mg) and the second product as chiral-N-methyl-2-(2-oxo-4-(o-tolyl)-2H-chromen-7-yl)cyclopropane-1-carboxamide (Example 136, Peak 2, 35 mg). The absolute stereochemistry of these Examples was not determined.

Chiral Prep method: SFC prep purification was performed on a SFC PREP 80 instrument by using (R,R) WHELK-01 (21.1×250 mm), 5μ operating at 35° C., maintaining flow rate of 60 gm/min., using 70% CO₂ in super critical state and 30% of MeOH as the mobile phase, held isocratic for up to 16 min. and isobaric at 100 bar at 220 nm wavelength.

Example 135: Chiral-N-methyl-2-(2-oxo-4-(o-tolyl)-2H-chromen-7-yl)cyclopropane-1-carboxamide, 3 (Peak 1): LCMS (ESI) Calcd. for C₂₁H₁₉O₃: 333, found [M+H]⁺=334. ¹H NMR (400 MHz, DMSO-d₆) δ 7.98 (t, 1H), 7.45-7.38 (m, 2H), 7.36 (t, 1H), 7.27 (t, 2H), 7.14 (d, 1H), 6.82 (d, 1H), 6.29 (s, 1H), 2.50-2.43 (m, 1H), 2.42 (d, 3H), 2.10 (s, 3H), 2.07 (t, 1H), 1.56 (t, 1H), 1.29-1.23 (m, 1H).

Example 136: Chiral-N-methyl-2-(2-oxo-4-(o-tolyl)-2H-chromen-7-yl)cyclopropane-1-carboxamide, 4 (Peak 2): LCMS (ESI) Calcd. for C₂₁H₁₉O₃: 333, found [M+H]⁺=334. ¹H NMR (400 MHz, DMSO-d₆) δ 7.98 (t, 1H), 7.45-7.38 (m, 2H), 7.36 (t, 1H), 7.27 (t, 2H), 7.14 (d, 1H), 6.82 (d, 1H), 6.29 (s, 1H), 2.50-2.43 (m, 1H), 2.42 (d, 3H), 2.10 (s, 3H), 2.07 (t, 1H), 1.56 (t, 1H), 1.29-1.23 (m, 1H).

### Examples 137-138: Synthesis of 3-((4-(2-chloro-4-fluorophenyl)-2-oxo-2H-chromen-7-yl)(methyl)amino)propenamide, 3-((4-(2-chloro-4-fluorophenyl)-2-oxo-2H-chromen-7-yl)(methyl)amino)-N-methylpropanamide

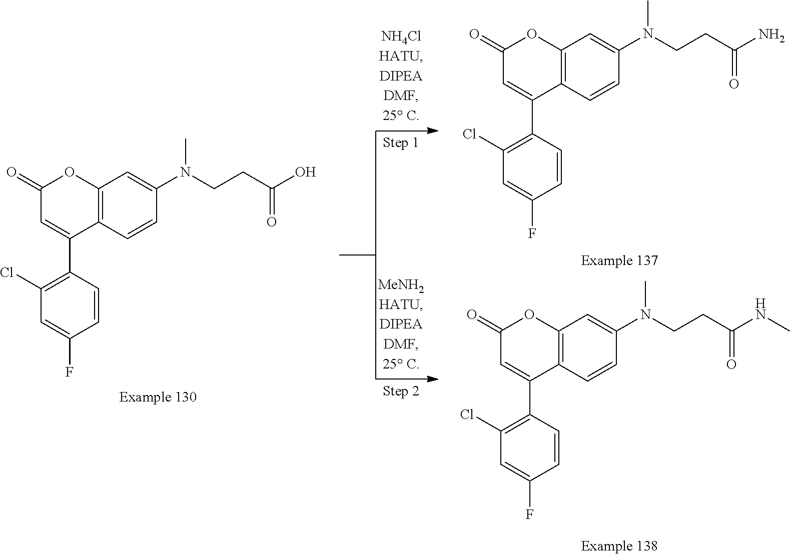 [SMILES: CN(CCC(=O)O)c1ccc2c(-c3ccc(F)cc3Cl)cc(=O)oc2c1 ; CNC(=O)CCN(C)c1ccc2c(-c3ccc(F)cc3Cl)cc(=O)oc2c1 ; CN(CCC(N)=O)c1ccc2c(-c3ccc(F)cc3Cl)cc(=O)oc2c1]

Synthesis of 3-((4-(2-chloro-4-fluorophenyl)-2-oxo-2H-chromen-7-yl)(methyl)amino)propenamide, Example 137 [Step 1]: To a stirred solution of 3-((4-(2-chloro-4-fluorophenyl)-2-oxo-2H-chromen-7-yl)(methyl)amino)propanoic acid (Example 130, 200 mg, 0.5 mmol) and ammonium chloride (425 mg, 8.0 mmol) in dry DMF (3 mL), HATU (335 mg, 0.9 mmol) and DIPEA (0.2 mL, 1.2 mmol) were added. After stirring for 12 h. at ambient temperature, the reaction mixture was quenched with water and extracted with ethyl acetate. The combined organic extract was washed with brine, dried over anhydrous Na₂SO₄, filtered and concentrated under reduced pressure. The product was purified by reverse phase prep-HPLC to afford 3-((4-(2-chloro-4-fluorophenyl)-2-oxo-2H-chromen-7-yl)(methyl)amino)propanamide (Example 137, 70 mg). LCMS (ESI) Calcd. for C₁₉H₁₆ClFN₂O₃: 374, found [M+H]⁺=375. ¹H NMR (400 MHz, DMSO-d₆) δ 7.69-7.67 (m, 1H), 7.54-7.51 (m, 1H), 7.43-7.39 (m, 2H), 6.88 (s, 1H), 6.78-6.75 (m, 1H), 6.68-6.67 (m, 2H), 6.00 (s, 1H), 3.65 (t, 2H) 2.98 (s, 3H), 2.32 (t, 2H).

Synthesis of 3-((4-(2-chloro-4-fluorophenyl)-2-oxo-2H-chromen-7-yl)(methyl)amino)-N-methylpropanamide, Example 138 [Step 2]: To a stirred solution of 3-((4-(2-chloro-4-fluorophenyl)-2-oxo-2H-chromen-7-yl)(methyl)amino)propanoic acid (Example 130, 200 mg, 0.5 mmol) and methylamine (2M in THF, 2.7 mL, 5.3 mmol) in dry DMF (3 mL), HATU (335 mg, 0.9 mmol) and DIPEA (0.2 mL, 1.2 mmol) were added successively. After stirring for 12 h. at ambient temperature, the reaction mixture was quenched with water and extracted with ethyl acetate. The combined organic extract was washed with brine, dried over anhydrous Na₂SO₄, filtered and concentrated under reduced pressure. The product was purified by reverse phase prep-HPLC to afford 3-((4-(2-chloro-4-fluorophenyl)-2-oxo-2H-chromen-7-yl)(methyl)amino)-N-methylpropanamide (Example 138, 50 mg). LCMS (ESI) Calcd. for C₂₀H₁₈ClFN₂O₃: 388, found [M+H]⁺=389. ¹H NMR (400 MHz, DMSO-d₆) δ 7.86 (br s, 1H), 7.69-7.67 (m, 1H), 7.54-7.50 (m, 1H), 7.43-7.39 (m, 1H), 6.77-6.75 (m, 1H), 6.68-6.66 (m, 2H), 6.00 (s, 1H), 3.65 (t, 2H) 2.96 (s, 3H), 2.55-2.54 (s, 3H), 2.32 (t, 2H).

### Example 139: Synthesis of 2-(methyl(2-oxo-4-(o-tolyl)-2H-chromen-7-yl)amino)-N-(tetrahydro-2H-pyran-4-yl)acetamide

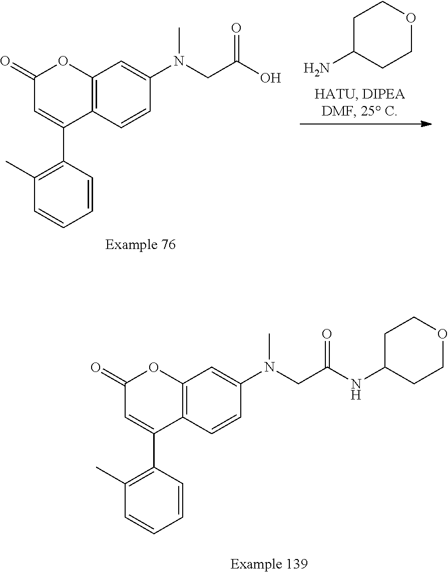 [SMILES: Cc1ccccc1-c1cc(=O)oc2cc(N(C)CC(=O)O)ccc12 ; NC1CCOCC1 ; Cc1ccccc1-c1cc(=O)oc2cc(N(C)CC(=O)NC3CCOCC3)ccc12]

Synthesis of 2-(methyl(2-oxo-4-(o-tolyl)-2H-chromen-7-yl)amino)-N-(tetrahydro-2H-pyran-4-yl)acetamide, Example 139: To a stirred solution of N-methyl-N-(2-oxo-4-(o-tolyl)-2H-chromen-7-yl)glycine (Example 76, 100 mg, 0.3 mmol) and tetrahydro-2H-pyran-4-amine (47 mg, 0.5 mmol) in DMF (2 mL) were added HATU (175 mg, 0.5 mmol) and DIPEA (0.1 mL, 0.6 mmol) and the reaction mixture was stirred at 25° C. After 16 h., the reaction mixture was quenched with cold water, and extracted with ethyl acetate. The combined organic extract was washed with brine, dried over anhydrous Na₂SO₄, filtered and concentrated under reduced pressure. The product was purified by reverse phase prep-HPLC to afford 2-(methyl(2-oxo-4-(o-tolyl)-2H-chromen-7-yl)amino)-N-(tetrahydro-2H-pyran-4-yl)acetamide (Example 139, 56 mg). LCMS (ESI) Calcd. for C₂₄H₂₆N₂O₄: 406, found [M+H]⁺=407. ¹H NMR (400 MHz, DMSO-d₆): δ 8.04-8.02 (m, 1H), 7.43-7.37 (m, 2H), 7.35-7.33 (m, 1H), 7.21-7.20 (m, 1H), 6.73-6.70 (m, 1H), 6.59-6.57 (m, 2H), 5.92 (s, 1H), 4.03 (s, 2H), 3.82-3.74 (m, 3H), 3.35-3.32 (m, 2H), 3.06 (s, 3H), 2.11 (s, 3H), 1.68-1.65 (m, 2H), 1.46-1.36 (m, 2H).

### Example 140: Synthesis of N-methyl-3-(methyl(2-oxo-4-(o-tolyl)-2H-chromen-7-yl)amino)propenamide

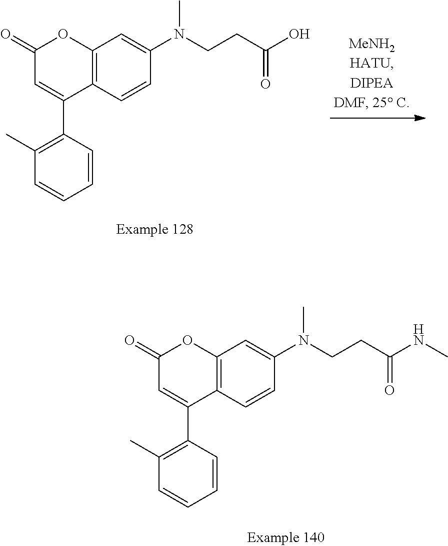 [SMILES: Cc1ccccc1-c1cc(=O)oc2cc(N(C)CCC(=O)O)ccc12 ; CNC(=O)CCN(C)c1ccc2c(-c3ccccc3C)cc(=O)oc2c1]

Synthesis of N-methyl-3-(methyl(2-oxo-4-(o-tolyl)-2H-chromen-7-yl)amino)propenamide, Example 140: To a stirred solution of 3-(methyl(2-oxo-4-(o-tolyl)-2H-chromen-7-yl)amino)propanoic acid (Example 128, 250 mg, 0.7 mmol) and methylamine (3.7 mL, 7.4 mmol, 2M in THF) in dry DMF (3 mL), HATU (465 mg, 1.2 mmol) and DIPEA (0.2 mL, 1.7 mmol) were added successively. After stirring for 12 h. at ambient temperature, the reaction mixture was quenched with water, and extracted with ethyl acetate. The combined organic extract was washed with brine, dried over anhydrous Na₂SO₄, filtered and concentrated under reduced pressure. The product was purified by reverse phase prep-HPLC to afford N-methyl-3-(methyl(2-oxo-4-(o-tolyl)-2H-chromen-7-yl)amino)propanamide (Example 140, 100 mg). LCMS (ESI) Calcd. for C₂₁H₂₂N₂O₃: 350, found [M+H]⁺=351. ¹H NMR (400 MHz, DMSO-d₆) δ 7.86-7.85 (m, 1H), 7.43-7.37 (m, 2H), 7.35-7.31 (m, 1H), 7.21-7.19 (m, 1H), 6.73-6.72 (m, 1H), 6.66-6.64 (m, 2H), 5.90 (s, 1H), 3.64 (t, 2H), 2.96 (s, 3H), 2.55-2.53 (m, 3H), 2.32 (t, 2H), 2.11 (s, 3H).

### Example 141: Synthesis of isopropyl N-methyl-N-(2-oxo-4-(o-tolyl)-2H-chromen-7-yl)glycinate

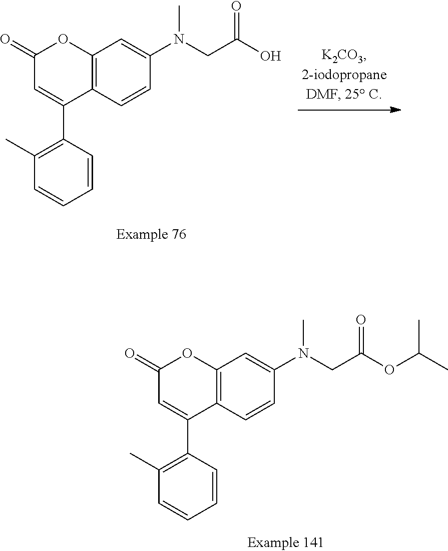 [SMILES: Cc1ccccc1-c1cc(=O)oc2cc(N(C)CC(=O)O)ccc12 ; Cc1ccccc1-c1cc(=O)oc2cc(N(C)CC(=O)OC(C)C)ccc12]

Synthesis of isopropyl N-methyl-N-(2-oxo-4-(o-tolyl)-2H-chromen-7-yl)glycinate, Example 141: To a solution of N-methyl-N-(2-oxo-4-(o-tolyl)-2H-chromen-7-yl)glycine (Example 76, 60 mg, 0.2 mmol) in DMF (1 mL) was added K₂CO₃ (50 mg, 0.4 mmol) followed by 2-iodopropane (0.06 mL, 0.6 mmol), and the reaction mixture was stirred at 25° C. for 16 h. The reaction mixture was quenched with cold water and extracted with EtOAc. The combined organic phase was dried over anhydrous Na₂SO₄, filtered and concentrated under reduced pressure. The compound was purified by RP prep-HPLC and lyophilized to afford isopropyl N-methyl-N-(2-oxo-4-(o-tolyl)-2H-chromen-7-yl)glycinate (Example 141, 45 mg). LCMS (ESI) Calcd. for C₂₂H₂₃NO₄: 365, found [M+H]⁺=365. ¹H NMR (400 MHz, DMSO-d₆) δ 7.43-7.37 (m, 2H), 7.35-7.33 (m, 1H), 7.22-7.21 (m, 1H), 6.74-6.72 (m, 1H), 6.65-6.61 (m, 2H), 5.95 (s, 1H), 4.95-4.89 (m, 1H), 4.29 (s, 2H), 3.05 (s, 3H), 2.11 (s, 3H), 1.18 (d, 6H).

### Example 142: Synthesis of ethyl N-methyl-N-(2-oxo-4-(o-tolyl)-2H-chromen-7-yl)glycinate

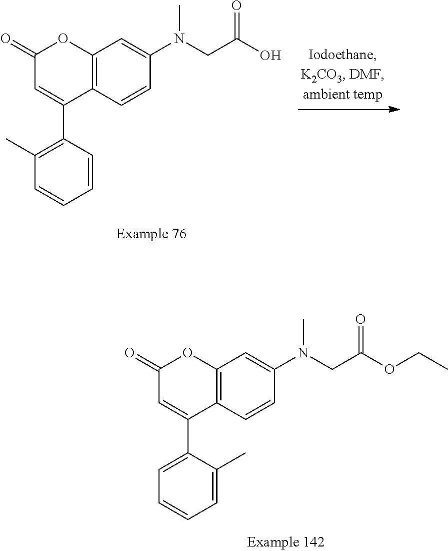 [SMILES: Cc1ccccc1-c1cc(=O)oc2cc(N(C)CC(=O)O)ccc12 ; CCOC(=O)CN(C)c1ccc2c(-c3ccccc3C)cc(=O)oc2c1]

Synthesis of ethyl N-methyl-N-(2-oxo-4-(o-tolyl)-2H-chromen-7-yl)glycinate, Example 142: To a stirred solution of N-methyl-N-(2-oxo-4-(o-tolyl)-2H-chromen-7-yl)glycine (Example 76, 60 mg, 0.2 mmol) in DMF (1 mL) was added K₂CO₃ (50 mg, 0.4 mmol) followed by iodoethane (0.04 mL, 0.6 mmol) and the reaction mixture was stirred at ambient temperature for 16 h. The reaction mixture was diluted with water, and extracted with ethyl acetate. The combined organic layer was washed with water and brine, dried over anhydrous Na₂SO₄, filtered and concentrated under reduced pressure. The product was purified by RP prep-HPLC and lyophilized to afford ethyl N-methyl-N-(2-oxo-4-(o-tolyl)-2H-chromen-7-yl)glycinate (Example 142, 28 mg). LCMS (ESI) Calcd. for C₂₁H₂₁NO₄: 351, found [M+H]⁺=352. ¹H NMR (400 MHz, DMSO-d₆) δ 7.41-7.38 (m, 2H), 7.35-7.31 (m, 1H), 7.22-7.20 (m, 1H), 6.74-6.71 (m, 1H), 6.67-6.62 (m, 2H), 5.95 (s, 1H), 4.34 (s, 2H), 4.14-4.08 (m, 2H), 3.05 (s, 3H), 2.11 (s, 3H), 1.20-1.16 (t, 3H).

### Example 143: Synthesis of methyl N-methyl-N-(2-oxo-4-(o-tolyl)-2H-chromen-7-yl)glycinate

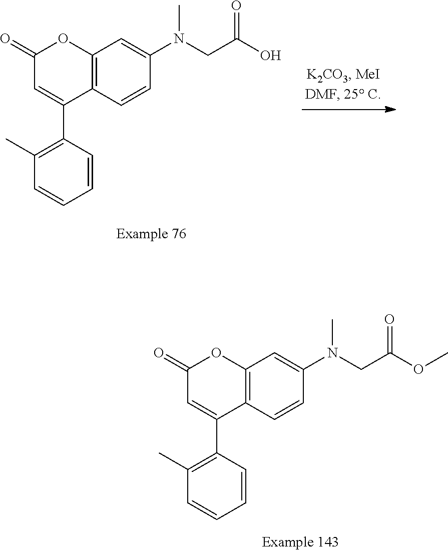 [SMILES: Cc1ccccc1-c1cc(=O)oc2cc(N(C)CC(=O)O)ccc12 ; COC(=O)CN(C)c1ccc2c(-c3ccccc3C)cc(=O)oc2c1]

Synthesis of methyl N-methyl-N-(2-oxo-4-(o-tolyl)-2H-chromen-7-yl)glycinate, Example 143: To a solution of N-methyl-N-(2-oxo-4-(o-tolyl)-2H-chromen-7-yl)glycine (Example 76, 60 mg, 0.2 mmol) in DMF (1 mL) was added K₂CO₃ (50 mg, 0.4 mmol) followed by iodomethane (0.04 mL, 0.6 mmol), and the reaction mixture was stirred at 25° C. for 2 h. The reaction mixture was quenched with cold water, and extracted with EtOAc. The combined organic phase was dried over anhydrous Na₂SO₄, filtered and concentrated under reduced pressure. The product was purified by RP prep-HPLC and lyophilized to afford methyl N-methyl-N-(2-oxo-4-(o-tolyl)-2H-chromen-7-yl)glycinate (Example 143, 25 mg). LCMS (ESI) Calcd. for C₂₀H₁₉NO₄: 337, found [M+H]⁺=338. ¹H NMR (400 MHz, DMSO-d₆) δ 7.41-7.38 (m, 2H), 7.34-7.33 (m, 1H), 7.22-7.20 (m, 1H), 6.74-6.72 (m, 1H), 6.67-6.63 (m, 2H), 5.95 (s, 1H), 4.36 (s, 2H), 3.65 (s, 3H), 3.05 (s, 3H), 2.11 (s, 3H).

### Example 144: Synthesis of 3-(methyl(2-oxo-4-(o-tolyl)-2H-chromen-7-yl)amino)propanoic acid

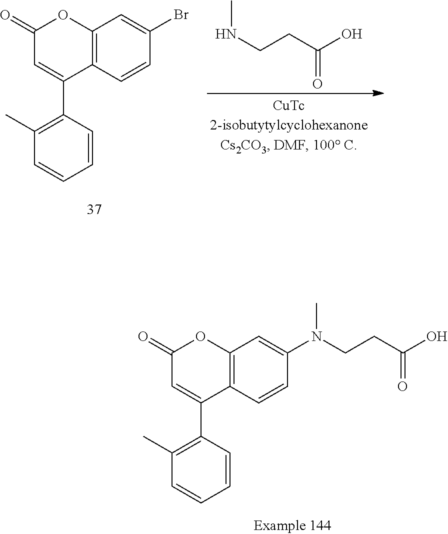

Synthesis of 3-(methyl(2-oxo-4-(o-tolyl)-2H-chromen-7-yl)amino)propanoic acid, Example 144: A sealed tube was charged with 7-bromo-4-(o-tolyl)-2H-chromen-2-one (37, 100 mg, 0.3 mmol), 3-(methylamino)propanoic acid (40 mg, 0.4 mmol), copper thiophene 2-carboxylate (12 mg, 0.1 mmol), 2-isobutyrylcyclohexanone (42 μL, 0.3 mmol), Cs₂CO₃ (205 mg, 0.6 mmol) and DMF (2 mL). The reaction vessel was flushed with argon gas and was tightly capped. The reaction tube was immersed into a heating block preheated to 100° C., and stirred for 12 h. After cooling to ambient temperature, the reaction mixture was acidified with 1N aq. HCl until pH=2, and extracted with 20% MeOH-DCM. The combined organic layer was washed with water and brine, dried over anhydrous Na₂SO₄, filtered and concentrated under reduced pressure. The product was purified using RP prep-HPLC and lyophilized to afford 3-(methyl(2-oxo-4-(o-tolyl)-2H-chromen-7-yl)amino)propanoic acid (Example 144, 38 mg). LCMS (ESI) Calcd. for C₂₀H₁₉NO₄: 337, found [M+H]⁺=338. ¹H NMR (400 MHz) δ 7.38-7.30 (m, 3H), 7.19-7.17 (m, 1H), 6.87-6.84 (m, 1H), 6.69-6.67 (m, 2H), 5.90 (s, 1H), 3.78-3.74 (m, 2H), 3.07 (s, 3H), 2.60-2.56 (m, 2H), 2.15 (s, 3H).

### Example 145: Synthesis of 3-((2-oxo-4-(o-tolyl)-2H-chromen-7-yl)amino)propanoic acid

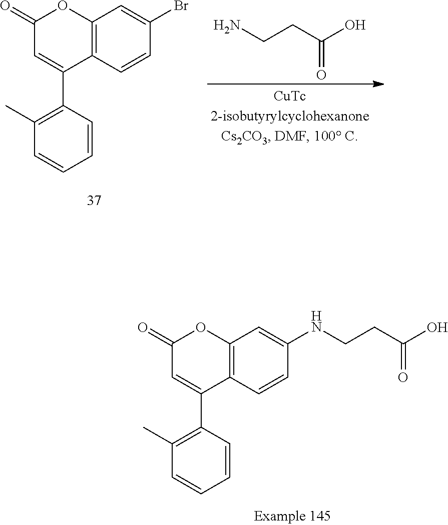 [SMILES: Cc1ccccc1-c1cc(=O)oc2cc(Br)ccc12 ; NCCC(=O)O ; Cc1ccccc1-c1cc(=O)oc2cc(NCCC(=O)O)ccc12]

Synthesis of 3-((2-oxo-4-(o-tolyl)-2H-chromen-7-yl)amino)propanoic acid, Example 145: A sealed tube was charged with 3-aminopropanoic acid (70 mg, 0.8 mmol), 7-bromo-4-(o-tolyl)-2H-chromen-2-one (37, 200 mg, 0.6 mmol), copper thiophene 2-carboxylate (25 mg, 0.1 mmol), 2-isobutyrylcyclohexanone (0.1 mL, 0.5 mmol), cesium carbonate (410 mg, 1.3 mmol) and DMF (3 mL). The reaction vessel was flushed with argon gas and was tightly capped. The sealed tube was heated in an aluminum block preheated to 100° C. After 16 h., the reaction mixture was quenched with water, and acidified using 2N aqueous HCl up to pH 5-6. The compound was extracted using 20% MeOH-DCM. The combined organic phase was dried over anhydrous Na₂SO₄, filtered and concentrated under reduced pressure. The compound was purified by RP prep-HPLC to afford 3-((2-oxo-4-(o-tolyl)-2H-chromen-7-yl)amino)propanoic acid (Example 145, 28 mg). LCMS (ESI) Calcd. for C₁₉H₁₇NO₄: 323, found [M−H]⁺=322. ¹H NMR (400 MHz, methanol-d₄): δ 7.38-7.30 (m, 3H), 7.18-7.16 (m, 1H), 6.76-6.74 (m, 1H), 6.55-6.51 (m, 2H), 5.86 (s, 1H), 3.46-3.44 (m, 2H), 2.58-2.60 (m, 2H), 2.15 (s, 3H).

### Examples 146-147: Synthesis of (S)-1-(methyl((2-oxo-4-(o-tolyl)-2H-chromen-7-yl)methyl)carbamoyl)piperidine-3-carboxylic acid, 1-(methyl((2-oxo-4-(o-tolyl)-2H-chromen-7-yl)methyl)carbamoyl)piperidine-4-carboxylic acid

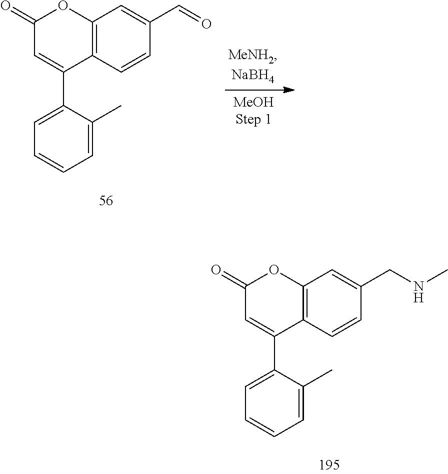 [SMILES: Cc1ccccc1-c1cc(=O)oc2cc(C=O)ccc12 ; CNCc1ccc2c(-c3ccccc3C)cc(=O)oc2c1]

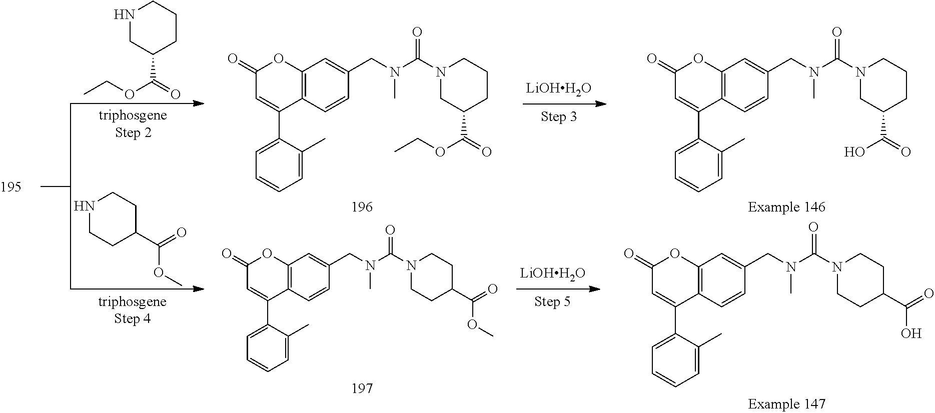

Synthesis of 7-((methylamino)methyl)-4-(o-tolyl)-2H-chromen-2-one, 195 [Step 1]: To a stirred solution of 2-oxo-4-(o-tolyl)-2H-chromene-7-carbaldehyde (56, 600 mg, 2.3 mmol) in methanol (5 mL) was added MeNH₂ in THF (11 ml, 22.7 mmol, 2M) followed by catalytic acetic acid. After 1 h, the reaction mixture was cooled at 0° C., NaBH₄ (335 mg, 9.1 mmol) was added and stirred at ambient temperature for 16 h. The reaction mixture was quenched with water and extracted with ethyl acetate. The combined organic layer was washed with brine, dried over anhydrous Na₂SO₄, filtered and concentrated under reduced pressure to afford 7-((methylamino)methyl)-4-(o-tolyl)-2H-chromen-2-one (195, 540 mg). LCMS (ESI) Calcd. for C₁₈H₁₇NO₂: 279, found [M+H]⁺=280.

Synthesis of ethyl (S)-1-(methyl((2-oxo-4-(o-tolyl)-2H-chromen-7-yl)methyl)carbamoyl)piperidine-3-carboxylate, 196 [Step 2]: To an ice cooled stirred solution of triphosgene (105 mg, 0.4 mmol) in DCM (5 mL), was added NaHCO₃ (0.2 mL, 2.2 mmol) in water (1 mL) followed by 7-((methylamino)methyl)-4-(o-tolyl)-2H-chromen-2-one (195, 200 mg, 0.7 mmol). After stirring for 1 h. at ambient temperature, the reaction mixture was diluted with DCM, washed with water and brine. The organic layer was dried over anhydrous Na₂SO₄, filtered and concentrated. The mixture was diluted in DCM (2 mL) and triethylamine (0.2 mL, 1.6 mmol) followed by ethyl (S)-piperidine-3-carboxylate (85 mg, 0.5 mmol) were added. The reaction mixture was stirred at ambient temperature for 12 h. After completion, reaction mixture was concentrated and purified by column chromatography to afford ethyl (S)-1-(methyl((2-oxo-4-(o-tolyl)-2H-chromen-7-yl)methyl)carbamoyl)piperidine-3-carboxylate (196, 180 mg). LCMS (ESI) Calcd. for C₂₇H₃₀N₂O₅: 462, found [M+H]⁺=463.

Synthesis of (S)-1-(methyl((2-oxo-4-(o-tolyl)-2H-chromen-7-yl)methyl)carbamoyl)piperidine-3-carboxylic acid, Example 146 [Step 3]: To a stirred solution of ethyl (S)-1-(methyl((2-oxo-4-(o-tolyl)-2H-chromen-7-yl)methyl)carbamoyl)piperidine-3-carboxylate (196, 150 mg, 0.3 mmol) in THF (4 mL), LiOH. H₂O (68 mg, 1.62 mmol) in water (1 mL) was added followed by methanol (2 mL). After stirring at ambient temperature for 16 h., the reaction mixture was concentrated under reduced pressure. The residue was acidified with 1N HCl and extracted with EtOAc. The combined organic layer was washed with brine, dried over anhydrous Na₂SO₄, filtered and concentrated under reduced pressure. The product was purified by RP prep-HPLC and lyophilized to afford(S)-1-(methyl((2-oxo-4-(o-tolyl)-2H-chromen-7-yl)methyl)carbamoyl)piperidine-3-carboxylic acid (Example 146, 80 mg). LCMS (ESI) Calcd. for C₂₅H₂₆N₂O₅: 434, found [M+H]⁺=435. ¹H NMR (400 MHz, DMSO-d₆ at 100° C.) δ 7.44-7.33 (m, 4H), 7.26 (d, 1H), 7.17 (d, 1H), 6.97 (d, 1H), 6.29 (s, 1H), 4.48-4.38 (m, 2H), 3.67-3.63 (dd, 1H), 3.45-3.42 (dd, 1H), 2.97-2.83 (m, 2H), 2.76 (s, 3H), 2.45-2.42 (m, 1H), 2.15 (s, 3H), 1.96-1.92 (m, 1H), 1.69-1.46 (m, 3H).

Synthesis of methyl 1-(methyl((2-oxo-4-(o-tolyl)-2H-chromen-7-yl)methyl)carbamoyl)piperidine-4-carboxylate, 197 [Step 4]: To an ice cooled stirred solution of triphosgene (105 mg, 0.4 mmol) in DCM (5 mL), was added NaHCO₃ (180 mg, 2.15 mmol) in water (1 mL) followed by 7-((methylamino)methyl)-4-(o-tolyl)-2H-chromen-2-one (195, 200 mg, 0.7 mmol). After stirring for 1 h. at ambient temperature, the reaction mixture was diluted with DCM, washed with water and brine. The organic layer was dried over anhydrous Na₂SO₄, filtered and concentrated. The residue was taken in DCM (2 mL) and triethylamine (0.2 mL, 1.6 mmol) and methyl piperidine-4-carboxylate (90 mg, 0.6 mmol) were added. The reaction mixture was stirred at ambient temperature for 12 h. After completion, reaction mixture was concentrated and purified by column chromatography to afford methyl 1-(methyl((2-oxo-4-(o-tolyl)-2H-chromen-7-yl)methyl)carbamoyl)piperidine-4-carboxylate (197, 180 mg). LCMS (ESI) Calcd. for C₂₆H₂₈N₂O₅: 448, found [M+H]⁺=449.

Synthesis of 1-(methyl((2-oxo-4-(o-tolyl)-2H-chromen-7-yl)methyl)carbamoyl)piperidine-4-carboxylic acid, Example 147 [Step 5]: To a stirred solution of methyl 1-(methyl((2-oxo-4-(o-tolyl)-2H-chromen-7-yl)methyl)carbamoyl)piperidine-4-carboxylate (197, 150 mg, 0.3 mmol) in THF (4 mL), LiOH. H₂O (68 mg, 1.62 mmol) in water (1 mL) was added followed by methanol (2 mL). After stirring at ambient temperature for 16 h., the reaction mixture was concentrated under reduced pressure. The residue was acidified with 1N HCl and extracted with EtOAc. The combined organic layer was washed with brine, dried over anhydrous Na₂SO₄, filtered and concentrated under reduced pressure. The product was purified by RP prep-HPLC and lyophilized to afford 1-(methyl((2-oxo-4-(o-tolyl)-2H-chromen-7-yl)methyl)carbamoyl)piperidine-4-carboxylic acid (Example 147, 14 mg). LCMS (ESI) Calcd. for C₂₅H₂₆N₂O₅: 434, found [M+H]⁺=435. ¹H NMR (400 MHz, DMSO-d₆) (at 100° C.) δ 7.44-7.33 (m, 4H), 7.26 (d, 1H), 7.17 (d, 1H), 6.97 (d, 1H), 6.29 (s, 1H), 4.42 (s, 2H), 3.56 (d, 2H), 2.89-2.83 (m, 2H), 2.77 (s, 3H), 2.42 (br s, 1H), 2.14 (s, 3H), 1.84-1.81 (m, 2H), 1.62-1.53 (m, 2H).

### Example 148: Synthesis of (R)-3-((2-oxo-4-(o-tolyl)-2H-chromen-7-yl)amino)butanoic acid

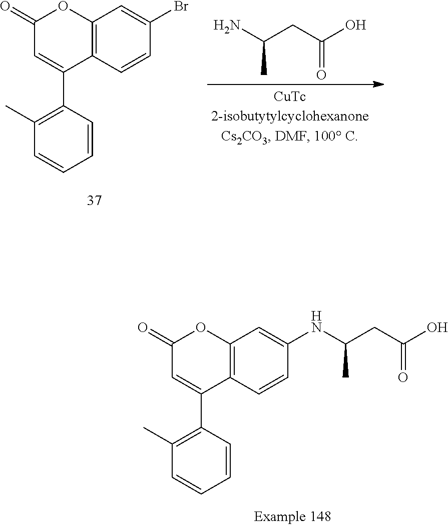

Synthesis of (R)-3-((2-oxo-4-(o-tolyl)-2H-chromen-7-yl)amino)butanoic acid, Example 148 [Step 1]: A sealed tube was charged with 7-bromo-4-(o-tolyl)-2H-chromen-2-one (37, 200 mg, 0.6 mmol), (R)-3-aminobutanoic acid (80 mg, 0.8 mmol), copper thiophene 2-carboxylate (24 mg, 0.1 mmol), 2-isobutyrylcyclohexanone (0.1 mL, 0.5 mmol), Cs₂CO₃ (415 mg, 1.3 mmol) and DMF (2 mL). The reaction vessel was flushed with argon gas and was tightly capped. The reaction tube was immersed into a heating block preheated to 100° C. and stirred for 12 h. After cooling to ambient temperature, the reaction mixture was acidified with 1N aq. HCl until pH 2, extracted with 20% MeOH-DCM. The combined organic layer was washed with water and brine, dried over anhydrous Na₂SO₄, filtered and concentrated under reduced pressure. The product was purified using RP prep-HPLC and lyophilized to afford (R)-3-((2-oxo-4-(o-tolyl)-2H-chromen-7-yl)amino)butanoic acid (Example 148, 50 mg). LCMS (ESI) Calcd. for C₂₀H₁₉NO₄: 337, found [M+H]⁺=338. ¹H NMR (400 MHz, MeOH-d₄) δ 7.38-7.28 (m, 3H), 7.18-7.16 (m, 1H) 6.76-6.73 (m, 1H), 6.55-6.51 (m, 2H), 5.86 (s, 1H), 4.03-3.99 (m, 2H), 2.64-2.58 (m, 1H), 2.44-2.38 (m, 1H) 2.15 (s, 3H), 1.29 (d, 3H).

### Example 149: Synthesis of (S)-3-((2-oxo-4-(o-tolyl)-2H-chromen-7-yl)amino)butanoic acid

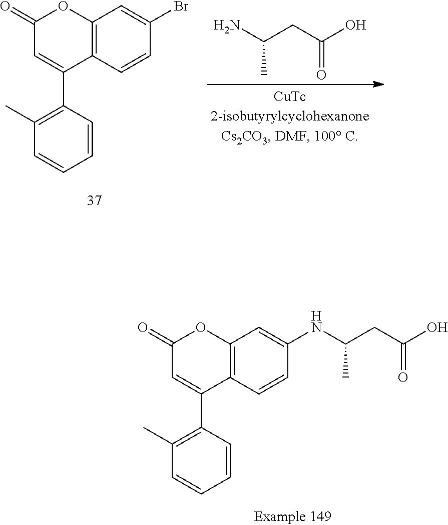

Synthesis of (S)-3-((2-oxo-4-(o-tolyl)-2H-chromen-7-yl)amino)butanoic acid, Example 149: A sealed tube was charged with 7-bromo-4-(o-tolyl)-2H-chromen-2-one (37, 200 mg, 0.6 mmol), (S)-3-aminobutanoic acid (80 mg, 0.78 mmol), copper thiophene 2-carboxylate (24 mg, 0.1 mmol), 2-isobutyrylcyclohexanone (0.1 mL, 0.5 mmol), cesium carbonate (410 mg, 1.3 mmol) and DMF (4 mL). The reaction vessel was flushed with argon, and immersed in a heating block preheated to 100° C. After 16 h., the reaction mixture was acidified using 2N aqueous HCl to pH 5-6 and extracted with 10% MeOH-DCM. The combined organic phase was washed with brine, dried over anhydrous sodium sulfate, filtered, and concentrated under reduced pressure. The compound was purified by RP prep-HPLC and lyophilized to afford(S)-3-((2-oxo-4-(o-tolyl)-2H-chromen-7-yl)amino)butanoic acid (Example 149, 42 mg). LCMS (ESI) Calcd. for C₂₀H₁₉NO₄: 337, found [M−H]⁺=336. ¹H NMR (400 MHz, methanol-d₄) δ 7.41-7.30 (m, 3H), 7.20-7.18 (m, 1H), 6.77-6.75 (m, 1H), 6.58-6.53 (m, 2H), 5.87 (s, 1H), 4.03-3.98 (m, 1H), 2.63-2.58 (m, 1H), 2.41-2.35 (m, 1H), 2.17 (s, 3H), 1.29 (d, 3H).

### Example 150: Synthesis of (S)-1-(((2-oxo-4-(o-tolyl)-2H-chromen-7-yl)methyl)carbamoyl)piperidine-3-carboxylic acid

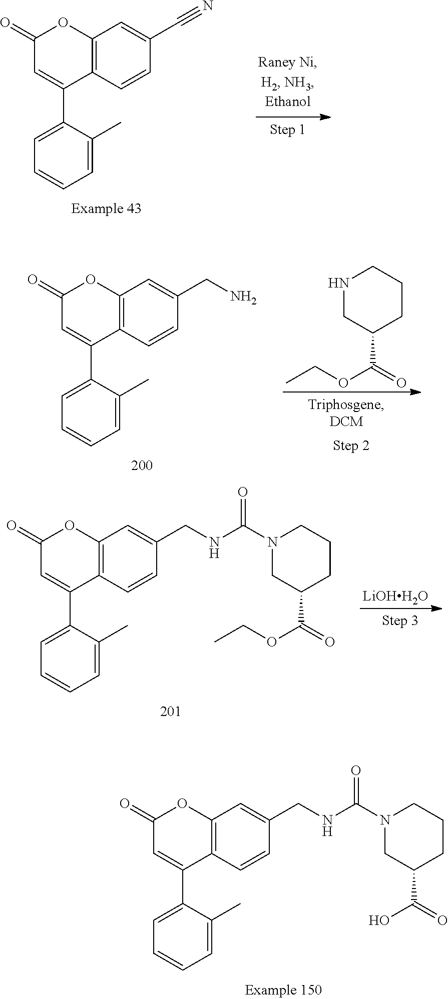

Synthesis of 7-(aminomethyl)-4-(o-tolyl)-2H-chromen-2-one, 200 [Step 1]: To a degassed solution of 2-oxo-4-(o-tolyl)-2H-chromene-7-carbonitrile (Example 43, 400 mg, 1.5 mmol) in ethanol (15 mL) was added Raney Ni (250 mg) followed by aqueous NH₃ (1 mL) and reaction mixture was stirred at ambient temperature for 16 h. under hydrogen balloon pressure. After completion, the reaction mixture was filtered through celite bed and concentrated under reduced pressure to afford 7-(aminomethyl)-4-(o-tolyl)-2H-chromen-2-one (200, 260 mg). LCMS (ESI) Calcd. for C₁₇H₁₅NO₂: 265, found [M+H]⁺=266.

Synthesis of ethyl (S)-1-(((2-oxo-4-(o-tolyl)-2H-chromen-7-yl)methyl)carbamoyl)piperidine-3-carboxylate, 201 [Step 2]: To a stirred solution of triphosgene (85 mg, 0.3 mmol) in DCM (6 mL) was added NaHCO₃ (0.2 mL, 1.7 mmol) in water (3 mL) dropwise at 0° C. followed by 7-(aminomethyl)-4-(o-tolyl) chromen-2-one (200, 150 mg, 0.6 mmol). The reaction mixture was stirred at ambient temperature for 1 h. After completion, the reaction mixture was diluted with DCM and washed with water and brine. The organic layer was dried over anhydrous Na₂SO₄ and concentrated under reduced pressure. The residue was taken in a DCM (4 mL) and triethylamine (0.2 mL, 1.1 mmol) followed by ethyl (S)-piperidine-3-carboxylate (70 mg, 0.5 mmol) were added. The reaction mixture was stirred at ambient temperature for 12 h. After completion, the reaction mixture was diluted with DCM and washed with water. The organic layer was dried over anhydrous Na₂SO₄ and concentrated under reduced pressure. The product was purified by flash column chromatography to yield ethyl (S)-1-(((2-oxo-4-(o-tolyl)-2H-chromen-7-yl)methyl)carbamoyl)piperidine-3-carboxylate (201, 112 mg). LCMS (ESI) Calcd. for C₂₆H₂₈N₂O₅: 448, found [M+H]⁺=449.

Synthesis of (S)-1-(((2-oxo-4-(o-tolyl)-2H-chromen-7-yl)methyl)carbamoyl)piperidine-3-carboxylic acid, Example 150 [Step 3]: To a solution of ethyl (S)-1-(((2-oxo-4-(o-tolyl)-2H-chromen-7-yl)methyl)carbamoyl)piperidine-3-carboxylate (201, 110 mg, 0.2 mmol) in THF (2 mL), methanol (1 mL) and water (0.5 mL) mixture was added LiOH·H₂O (50 mg, 1.2 mmol) and reaction mixture was stirred at ambient temperature for 16 h. The reaction mixture was concentrated under reduced pressure. The residue was acidified with 1N HCl to pH of 3-4 and was extracted with ethyl acetate. The combined organic layer was washed with brine, dried over anhydrous Na₂SO₄, filtered and concentrated under reduced pressure. The product was purified through RP prep-HPLC and lyophilized to afford(S)-1-(((2-oxo-4-(o-tolyl)-2H-chromen-7-yl)methyl)carbamoyl)piperidine-3-carboxylic acid (Example 150, 48 mg). LCMS (ESI) Calcd. for C₂₄H₂₄N₂O₅: 420, found [M+H]⁺=421. ¹H NMR (400 MHz, DMSO-d₆) at 100° C. δ 7.44-7.41 (m, 2H), 7.37-7.33 (m, 2H), 7.25 (d, 1H), 7.19 (d, 1H), 6.94 (d, 2H), 6.25 (s, 1H), 4.35 (s, 2H), 4.01 (d, 1H), 3.78 (d, 1H), 2.99-2.84 (m, 2H), 2.33 (s, 1H), 2.14 (s, 3H), 1.97 (d, 1H), 1.65-1.55 (m, 2H), 1.42 (d, 1H).

### Example 151: Synthesis of 1-(((2-oxo-4-(o-tolyl)-2H-chromen-7-yl)methyl)carbamoyl)piperidine-4-carboxylic acid

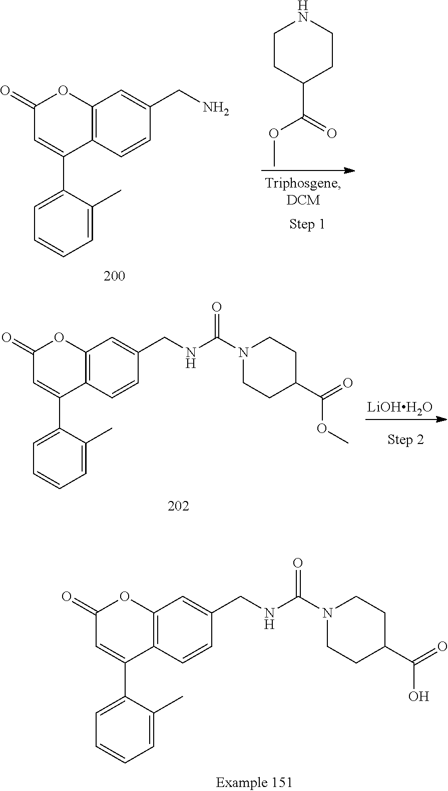

Synthesis of methyl 1-(((2-oxo-4-(o-tolyl)-2H-chromen-7-yl)methyl)carbamoyl)piperidine-4-carboxylate, 202 [Step 1]: To an ice cold stirred solution of triphosgene (85 mg, 0.3 mmol) in DCM (5 mL), was added NaHCO₃ (140 mg, 1.7 mmol) in water (1 mL) followed by 7-(aminomethyl)-4-(o-tolyl)-2H-chromen-2-one (200, 150 mg, 0.7 mmol). After stirring for 1 h. at ambient temperature, the reaction mixture was diluted with DCM, washed with water and brine. The organic layer was dried over anhydrous Na₂SO₄, filtered, and concentrated under reduced pressure. The residue was diluted in DCM (2 mL), and triethylamine (0.16 mL, 1.14 mmol) and methyl piperidine-4-carboxylate (66 mg, 0.45 mmol) were added. The reaction mixture was stirred at ambient temperature for 12 h. After completion, the reaction mixture was concentrated and purified by column chromatography to afford methyl 1-(((2-oxo-4-(o-tolyl)-2H-chromen-7-yl)methyl)carbamoyl)piperidine-4-carboxylate (202, 80 mg). LCMS (ESI) Calcd. for C₂₅H₂₆N₂O₅: 434, found [M+H]⁺=435.

Synthesis of 1-(((2-oxo-4-(o-tolyl)-2H-chromen-7-yl)methyl)carbamoyl)piperidine-4-carboxylic acid, Example 151 [Step 2]: To a stirred solution of methyl 1-(((2-oxo-4-(o-tolyl)-2H-chromen-7-yl)methyl)carbamoyl)piperidine-4-carboxylate (202, 70 mg, 0.16 mmol) in THF (2 mL), methanol (1 mL) and water (0.5 mL) mixture, was added LiOH· H₂O (34 mg, 0.81 mmol). After stirring at ambient temperature for 16 h., the reaction mixture was concentrated under reduced pressure. The residue was acidified with 1N HCl and extracted with EtOAc. The combined organic layer was washed with brine, dried over anhydrous Na₂SO₄, filtered, and concentrated under reduced pressure. The product was purified through RP prep-HPLC and lyophilized to afford 1-(((2-oxo-4-(o-tolyl)-2H-chromen-7-yl)methyl)carbamoyl)piperidine-4-carboxylic acid (Example 151, 26 mg). LCMS (ESI) Calcd. for C₂₄H₂₄N₂O₅: 420, found [M+H]⁺=421. ¹H NMR (400 MHz, DMSO-d₆) δ 7.46-7.39 (m, 2H), 7.37-7.31 (m, 2H), 7.25 (d, 1H) 7.20-7.16 (m, 2H), 6.92 (d, 1H), 6.32 (s, 1H), 4.30 (d, 2H), 3.88 (d, 2H), 2.84-2.78 (m, 2H), 2.41-2.33 (m, 1H), 2.11 (s, 3H), 1.76 (d, 2H), 1.45-1.36 (m, 2H).

### Example 152: Synthesis of (1r,4r)-4-(((2-oxo-4-(o-tolyl)-2H-chromen-7-yl)methyl)carbamoyl)cyclohexane-1-carboxylic acid

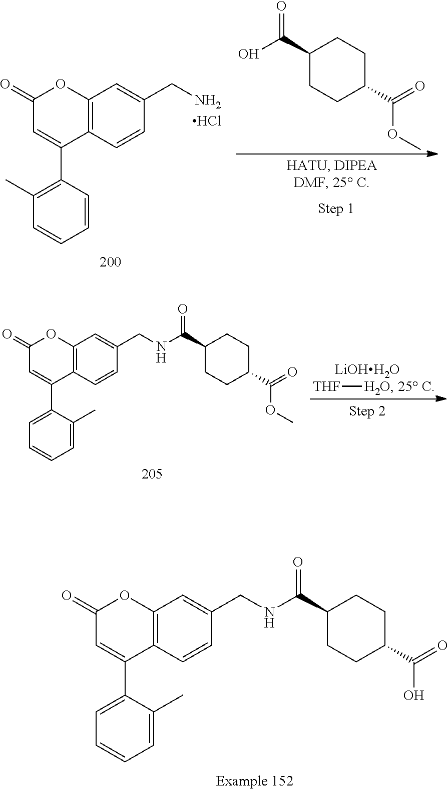

Synthesis of methyl(1R,4R)-4-(((2-oxo-4-(o-tolyl)-2H-chromen-7-yl)methyl)carbamoyl)cyclohexane-1-carboxylate, 205 [Step 1]: To a stirring solution of (1R,4R)-4-(methoxycarbonyl)cyclohexane-1-carboxylic acid (245 mg, 1.3 mmol) in DMF (5 mL) were added HATU (455 mg, 1.2 mmol), 7-(aminomethyl)-4-(o-tolyl)-2H-chromen-2-one hydrochloride (200, 330 mg, 1.1 mmol) and DIPEA (0.3 mL, 1.6 mmol). The reaction mixture was stirred at 25° C. for 2 h. The reaction mixture was diluted with 10% MeOH in DCM and washed with water and brine. The organic phase was dried over anhydrous Na₂SO₄, filtered and concentrated under reduced pressure to afford (1R,4R)-4-(((2-oxo-4-(o-tolyl)-2H-chromen-7-yl)methyl)carbamoyl)cyclohexane-1-carboxylate (205, 400 mg). LCMS (ESI) Calcd. for C₂₆H₂₇NO₅: 433, found [M+H]⁺=434.

Synthesis of (1R,4R)-4-(((2-oxo-4-(o-tolyl)-2H-chromen-7-yl)methyl)carbamoyl)cyclohexane-1-carboxylic acid, Example 152 [Step 2]: To a solution of (1R,4R)-4-(((2-oxo-4-(o-tolyl)-2H-chromen-7-yl)methyl)carbamoyl)cyclohexane-1-carboxylate (205, 200 mg, 0.5 mmol) in THF (2 ml) and water (1 ml) mixture was added LiOH. H₂O (30 mg, 0.7 mmol). The reaction mixture was stirred at ambient temperature for 16 h. The resulting mixture was concentrated under reduced pressure and acidified with 1N HCl and was extracted with ethyl acetate. The combined organic layer was washed with brine, dried over anhydrous Na₂SO₄, filtered, and concentrated under reduced pressure. The product was purified by RP prep-HPLC and lyophilized to afford (1R,4R)-4-(((2-oxo-4-(o-tolyl)-2H-chromen-7-yl)methyl)carbamoyl)cyclohexane-1-carboxylic acid (Example 152, 60 mg). LCMS (ESI) Calcd. for C₂₅H₂₅NO₅: 419, found [M+H]⁺=420. ¹H NMR (400 MHz, methanol-d₄): δ 7.43-7.31 (m, 4H), 7.22-7.20 (m, 1H), 7.17-7.15 (m, 1H), 7.02-7.00 (m, 1H), 6.28 (s, 1H), 4.44 (s, 2H), 2.28-2.22 (m, 2H), 2.14 (m, 3H), 2.06-2.03 (m, 2H), 1.93-1.89 (m, 2H), 1.57-1.39 (m, 4H)

### Example 153: Synthesis of 4-(((2-oxo-4-(o-tolyl)-2H-chromen-7-yl)methyl)carbamoyl)cyclohexane-1-carboxylic acid

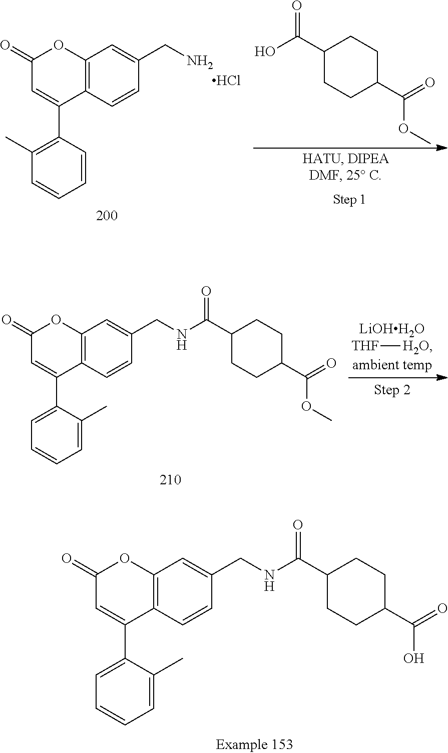

Synthesis of methyl 4-(((2-oxo-4-(o-tolyl)-2H-chromen-7-yl)methyl)carbamoyl)cyclohexane-1-carboxylate, 210 [Step 1]: To a stirring solution of 4-(methoxycarbonyl)cyclohexane-1-carboxylic acid (245 mg, 1.3 mmol) in DMF (5 mL) were added HATU (455 mg, 1.2 mmol), 7-(aminomethyl)-4-(o-tolyl)-2H-chromen-2-one hydrochloride (200, 330 mg, 1.1 mmol) and DIPEA (0.3 mL, 1.6 mmol). The reaction mixture was stirred at ambient temperature for 2 h. The reaction mixture was diluted with ethyl acetate and washed with cold water and cold brine. The organic layer was dried over anhydrous Na₂SO₄, filtered, and concentrated under reduced pressure to afford methyl 4-(((2-oxo-4-(o-tolyl)-2H-chromen-7-yl)methyl)carbamoyl)cyclohexane-1-carboxylate (210, 450 mg). LCMS (ESI) Calcd. for C₂₆H₂₇NO₅: 433, found [M+H]⁺=434.

Synthesis of 4-(((2-oxo-4-(o-tolyl)-2H-chromen-7-yl)methyl)carbamoyl)cyclohexane-1-carboxylic acid, Example 153 [Step 2]: To a solution of methyl 4-(((2-oxo-4-(o-tolyl)-2H-chromen-7-yl)methyl)carbamoyl)cyclohexane-1-carboxylate (210, 120 mg, 0.3 mmol) in THF (4 mL) and water (1 mL) mixture was added LiOH·H₂O (115 mg, 2.8 mmol). The reaction mixture was stirred at ambient temperature for 16 h. The resulting mixture was diluted with water and extracted with ethyl acetate. The water layer was collected, acidified with 1N HCl to pH 2-3 and extracted with ethyl acetate. The collected organic layer was washed with water and brine, dried over anhydrous Na₂SO₄, filtered, and concentrated under reduced pressure. The product was purified using RP prep-HPLC and lyophilized to afford 4-(((2-oxo-4-(o-tolyl)-2H-chromen-7-yl)methyl)carbamoyl)cyclohexane-1-carboxylic acid (Example 153, 22 mg). LCMS (ESI) Calcd. for C₂₅H₂₅NO₅: 419, found [M+H]⁺=420. ¹H NMR (400 MHz DMSO-d₆) δ 12.02 (s, 1H), 8.42-8.39 (m, 1H), 7.46-7.33 (m, 3H) 7.29-7.23 (m, 2H), 7.15-7.12 (m, 1H), 6.94-6.91 (m, 1H), 6.27 (s, 1H), 4.34-4.33 (m, 2H), 2.19-2.13 (m, 2H), 2.11 (s, 3H), 1.95-1.91 (m, 2H), 1.83-1.79 (m, 2H), 1.45-1.23 (m, 4H).

### Examples 154-155: Synthesis of 3-(methyl(2-oxo-4-(o-tolyl)-2H-chromen-7-yl)amino)butanoic acid, 2-methyl-3-(methyl(2-oxo-4-(o-tolyl)-2H-chromen-7-yl)amino)propanoic acid

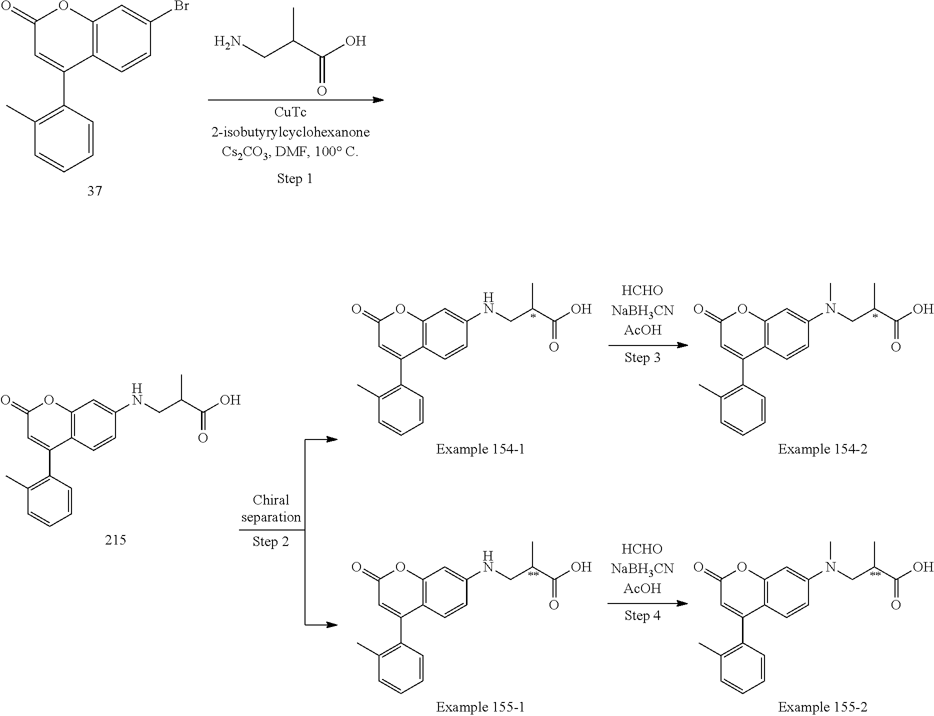

Synthesis of 2-methyl-3-((2-oxo-4-(o-tolyl)-2H-chromen-7-yl)amino)propanoic acid, 215 [Step 1]: A sealed tube was charged with 7-bromo-4-(o-tolyl)-2H-chromen-2-one (37, 750 mg, 2.4 mmol), 3-amino-2-methylpropanoic acid (295 mg, 2.9 mmol), copper thiophene 2-carboxylate (90 mg, 0.5 mmol), 2-isobutyrylcyclohexanone (0.3 mL, 1.9 mmol), cesium carbonate (1.6 g, 4.8 mmol) and DMF (8 mL). The reaction vessel was flushed with argon gas and was tightly capped. The reaction tube was immersed into a heating block preheated to 100° C. After 16 h., the reaction mixture was cooled to ambient temperature, acidified with 2N aqueous HCl to pH 4-5 and extracted with 20% MeOH in DCM. The combined organic phase was dried over anhydrous Na₂SO₄, filtered, and concentrated under reduced pressure. The product was purified by RP prep-HPLC to afford 2-methyl-3-((2-oxo-4-(o-tolyl)-2H-chromen-7-yl)amino)propanoic acid (215, 300 mg). LCMS (ESI) Calcd. for C₂₀H₁₉NO₄: 337, found [M+H]⁺=338.

Isolation of isomers of 2-methyl-3-((2-oxo-4-(o-tolyl)-2H-chromen-7-yl)amino)propanoic acid, Example 154-1 and Example 155-1 [Step 2]: Chiral separation of racemic 2-methyl-3-((2-oxo-4-(o-tolyl)-2H-chromen-7-yl)amino)propanoic acid (215, 300 mg, 0.9 mmol) was carried out by NP chiral purification, and the obtained two fractions were separately purified again by prep-HPLC(RP) to afford 2-methyl-3-((2-oxo-4-(o-tolyl)-2H-chromen-7-yl)amino)propanoic acid (216, 110 mg) as Peak 1 and 2-methyl-3-((2-oxo-4-(o-tolyl)-2H-chromen-7-yl)amino)propanoic acid (217, 120 mg) as Peak 2.

Peak 1:2-methyl-3-((2-oxo-4-(o-tolyl)-2H-chromen-7-yl)amino)propanoic acid, 216: LCMS (ESI) Calcd. for C₂₀H₁₉NO₄: 337, found [M+H]⁺=338. ¹H NMR (400 MHz, methanol-d₄) δ 7.37-7.30 (m, 3H), 7.18-7.16 (m, 1H), 6.75-6.73 (m, 1H), 6.55-6.51 (m, 2H), 5.86 (s, 1H), 3.46-3.44 (m, 1H), 3.25-3.21 (m, 1H), 2.73-2.72 (m, 1H), 2.15 (s, 3H), 1.21 (d, 3H).

Peak 2:2-methyl-3-((2-oxo-4-(o-tolyl)-2H-chromen-7-yl)amino)propanoic acid, 217: LCMS (ESI) Calcd. for C₂₀H₁₉NO₄: 337, found [M+H]⁺=338. ¹H NMR (400 MHz, methanol-d₄) δ 7.39-7.28 (m, 3H), 7.18-7.16 (m, 1H), 6.75-6.73 (m, 1H), 6.55-6.52 (m, 2H), 5.86 (s, 1H), 3.50-3.44 (m, 1H), 3.25-3.20 (m, 1H), 2.76-2.72 (m, 1H), 2.15 (s, 3H), 1.21 (d, 3H).

Chiral HPLC purification method: Chiral separation was performed on an Agilent 1200 series instrument. Column was a CHIRALPAK IG (250×21 mm) 5μ. Operating at ambient temperature and flow rate is 21.0 mL/min. Mobile phase was mixture of 70% Hexane, 15% DCM, 15% Isopropyl alcohol and 0.1% TFA held this isocratic mixture run for up to 20 min. with detection at 362 nm wavelength.

Synthesis of 3-(methyl(2-oxo-4-(o-tolyl)-2H-chromen-7-yl)amino)butanoic acid, Example 154-2 [Step 3]: To an ice-cold solution of (R)-3-((2-oxo-4-(o-tolyl)-2H-chromen-7-yl)amino)butanoic acid (Example 154-1, 80 mg, 0.2 mmol) and formaldehyde, 37% in water (0.1 mL, 2.4 mmol) in acetic acid (2 mL) was added sodium cyanoborohydride (45 mg, 0.7 mmol), and the reaction mixture was stirred at ambient temperature for 30 min. The reaction mixture was concentrated under reduced pressure and the product was purified by reverse phase prep-HPLC to afford 3-(methyl(2-oxo-4-(o-tolyl)-2H-chromen-7-yl)amino)butanoic acid (Example 154-2, 26 mg). LCMS (ESI) Calcd. for C₂₁H₂₁NO₄: 351, found [M+H]⁺=352. ¹H NMR (400 MHz DMSO-d₆) δ 7.41-7.36 (m, 2H), 7.34-7.30 (m, 1H), 7.22-7.19 (m, 1H), 6.72-6.70 (m, 1H), 6.66-6.63 (m, 2H), 5.89 (s, 1H), 3.64-3.62 (m, 1H), 3.42-3.38 (m, 1H), 2.99 (s, 3H), 2.67-2.65 (m, 1H), 2.11 (s, 3H), 1.04 (d, 3H).

Synthesis of 2-methyl-3-(methyl(2-oxo-4-(o-tolyl)-2H-chromen-7-yl)amino)propanoic acid, Example 155-2 [Step 4]: To an ice-cold solution of 2-methyl-3-((2-oxo-4-(o-tolyl)-2H-chromen-7-yl)amino)propanoic acid (Example 155-1, 100 mg, 0.3 mmol) and formaldehyde, 37% in water (0.1 mL, 2.9 mmol) in acetic acid (2 mL) was added sodium cyanoborohydride (55 mg, 0.9 mmol), and the reaction mixture was stirred at ambient temperature for 30 min. The reaction mixture was concentrated under reduced pressure, and the product was purified by reverse phase prep-HPLC to afford 2-methyl-3-(methyl(2-oxo-4-(o-tolyl)-2H-chromen-7-yl)amino)propanoic acid (Example 155-2, 60 mg). LCMS (ESI) Calcd. for C₂₁H₂₁NO₄: 351, found [M+H]⁺=352. ¹H NMR (400 MHz, methanol-d₄) δ 7.40-7.29 (m, 3H), 7.19-7.17 (m, 1H), 6.86-6.84 (m, 1H), 6.69-6.66 (m, 2H), 5.90 (s, 1H), 3.78-3.73 (m, 1H), 3.53-3.48 (m, 1H), 3.12 (s, 3H), 2.90-2.85 (m, 1H), 2.15 (s, 3H), 1.16 (d, 3H).

### Example 156: Synthesis of (1R,4R)-4-(methyl((2-oxo-4-(o-tolyl)-2H-chromen-7-yl)methyl)carbamoyl)cyclohexane-1-carboxylic acid

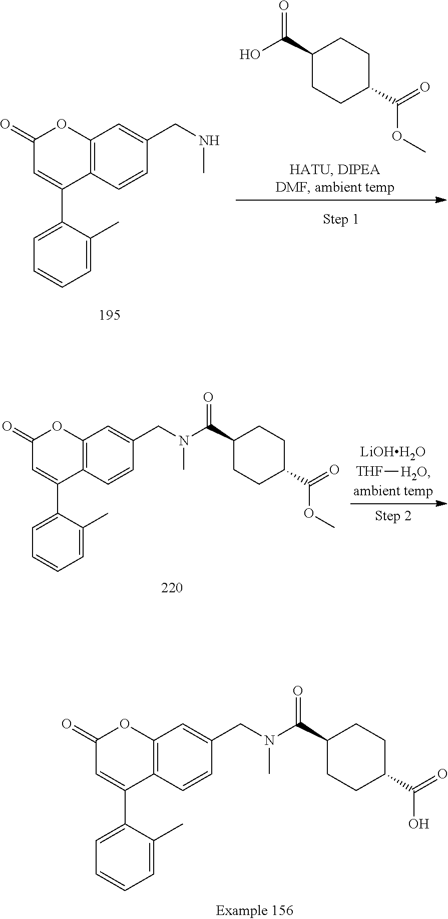

Synthesis of methyl(1R,4R)-4-(methyl((2-oxo-4-(o-tolyl)-2H-chromen-7-yl)methyl)carbamoyl)cyclohexane-1-carboxylate, 220 [Step 1]: To a stirred solution of (1R,4R)-4-(methoxycarbonyl)cyclohexane-1-carboxylic acid (104 mg, 0.6 mmol) in DMF (5 mL) were added HATU (195 mg, 0.5 mmol), 7-((methylamino)methyl)-4-(o-tolyl)-2H-chromen-2-one (195, 130 mg, 0.5 mmol) and DIPEA (0.12 mL, 0.7 mmol). The reaction mixture was stirred at ambient temperature for 2 h. The reaction mixture was diluted with 10% MeOH in DCM and washed with water followed by brine. The organic layer was dried over anhydrous, Na₂SO₄, filtered, and concentrated under reduced pressure to afford methyl(1R,4R)-4-(methyl((2-oxo-4-(o-tolyl)-2H-chromen-7-yl)methyl)carbamoyl)cyclohexane-1-carboxylate (220, 250 mg). LCMS (ESI) Calcd. for C₂₇H₂₉NO₅: 447, found [M+H]⁺=448.

Synthesis of (1R,4R)-4-(methyl((2-oxo-4-(o-tolyl)-2H-chromen-7-yl)methyl)carbamoyl)cyclohexane-1-carboxylic acid, Example 156 [Step 2]: To a solution of methyl(1R,4R)-4-(methyl((2-oxo-4-(o-tolyl)-2H-chromen-7-yl)methyl)carbamoyl)cyclohexane-1-carboxylate (220, 250 mg, 0.6 mmol) in THF (2 mL) and water (1 mL) mixture was added LiOH. H₂O (70 mg, 1.7 mmol). The reaction mixture was stirred for at 25° C. for 16 h. The resulting mixture was concentrated under reduced pressure. The mixture was acidified with 1N HCl to pH 2-3 and was extracted with ethyl acetate. The combined organic layer was washed with brine, dried over anhydrous Na₂SO₄, filtered, and concentrated under reduced pressure to afford (1R,4R)-4-(methyl((2-oxo-4-(o-tolyl)-2H-chromen-7-yl)methyl)carbamoyl)cyclohexane-1-carboxylic acid (Example 156, 70 mg). LCMS (ESI) Calcd. for C₂₆H₂₇NO₅: 433, found [M+H]⁺=434. ¹H NMR (400 MHz, DMSO-d₆ at 100° C.): δ 7.46-7.39 (m, 2H), 7.37-7.34 (m, 1H), 7.26-7.25 (m, 2H), 7.11-7.09 (d, 1H), 6.98-6.96 (d, 1H), 6.28 (s, 1H), 4.64 (s, 2H), 3.01 (s, 3H), 2.67-2.63 (m, 1H), 2.20 (m, 1H), 2.14 (s, 3H), 1.96-1.94 (m, 2H), 1.77 (m, 2H), 1.52-1.39 (m, 4H).

### Example 157: Synthesis of (1S,4S)-4-(((2-oxo-4-(o-tolyl)-2H-chromen-7-yl)methyl)carbamoyl)cyclohexane-1-carboxylic acid

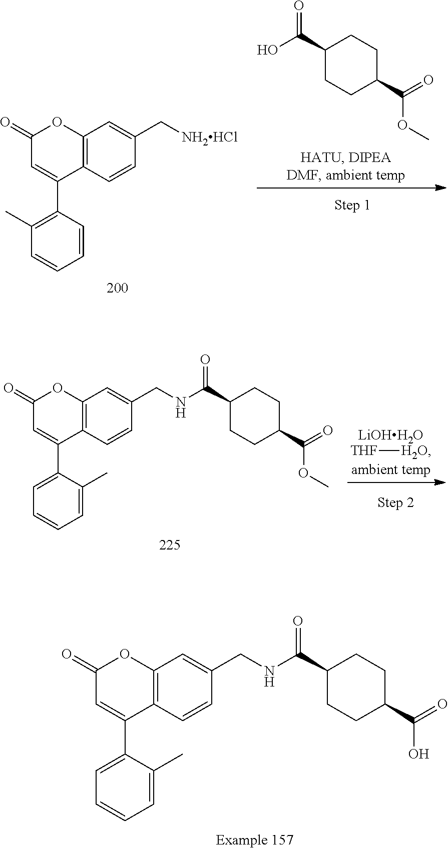

Synthesis of methyl(1S,4S)-4-(((2-oxo-4-(o-tolyl)-2H-chromen-7-yl)methyl)carbamoyl)cyclohexane-1-carboxylate, 225 [Step 1]: To a stirring solution of 7-(aminomethyl)-4-(o-tolyl) chromen-2-one hydrochloride (200, 330 mg, 1.1 mmol) and (1S,4S)-4-(methoxycarbonyl)cyclohexane-1-carboxylic acid (244 mg, 1.3 mmol) in DMF (5 mL) was added HATU (457 mg, 1.2 mmol) followed by DIPEA (0.3 mL, 1.6 mmol), and the reaction mixture was stirred at ambient temperature. After 16 h., the reaction mixture was quenched with chilled water and extracted with EtOAc. The combined organic phase was dried over anhydrous Na₂SO₄, filtered, and concentrated under reduced pressure to afford methyl(1S,4S)-4-(((2-oxo-4-(o-tolyl)-2H-chromen-7-yl)methyl)carbamoyl)cyclohexane-1-carboxylate (225, 400 mg). LCMS (ESI) Calcd. for C₂₆H₂₇NO₅: 433, found [M+H]⁺=434.

Synthesis of (1S,4S)-4-(((2-oxo-4-(o-tolyl)-2H-chromen-7-yl)methyl)carbamoyl)cyclohexane-1-carboxylic acid, Example 157 [Step 2]: To a solution of methyl(1S,4S)-4-(((2-oxo-4-(o-tolyl)-2H-chromen-7-yl)methyl)carbamoyl)cyclohexane-1-carboxylate (225, 250 mg, 0.6 mmol) in THF (3 mL) water (0.6 mL) mixture was added lithium hydroxide monohydrate (35 mg, 0.9 mmol), and the reaction mixture was stirred at ambient temperature. After 16 h., the reaction mixture was concentrated under reduced pressure. The residue was acidified with 1N aqueous HCl to pH 2-3 and extracted with 20% MeOH-DCM. The combined organic phase was dried over anhydrous Na₂SO₄, filtered, and concentrated under reduced pressure. The product was purified by RP prep-HPLC to afford (1S,4S)-4-(((2-oxo-4-(o-tolyl)-2H-chromen-7-yl)methyl)carbamoyl)cyclohexane-1-carboxylic acid (Example 157, 70 mg, racemic). LCMS (ESI) Calcd. for C₂₅H₂₅NO₅: 419, found [M+H]⁺=420. ¹H NMR (400 MHz, methanol-d₄): δ 7.43-7.31 (m, 4H), 7.22-7.20 (m, 1H), 7.16-7.14 (m, 1H), 7.02-7.00 (m, 1H), 6.27 (s, 1H), 4.44 (s, 2H), 2.56-2.54 (m, 1H), 2.36-2.31 (m, 1H), 2.13-2.10 (m, 5H), 1.78-1.56 (m, 6H).

### Example 158: Synthesis of (1S,4S)-4-(methyl((2-oxo-4-(o-tolyl)-2H-chromen-7-yl)methyl)carbamoyl)cyclohexane-1-carboxylic acid

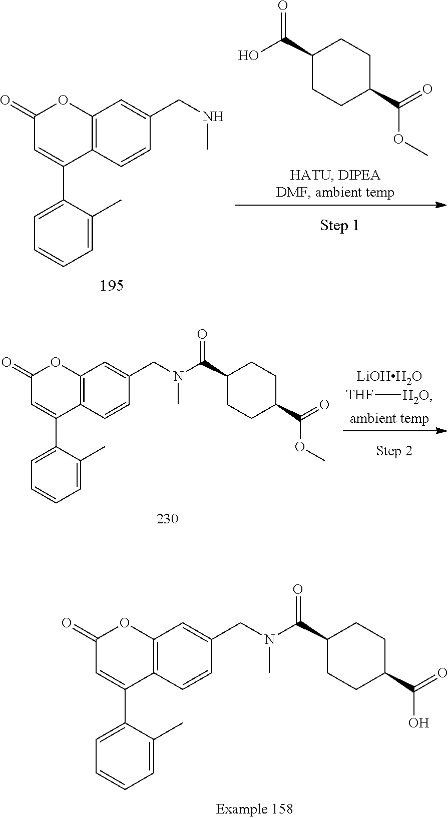

Synthesis of methyl(1S,4S)-4-(methyl((2-oxo-4-(o-tolyl)-2H-chromen-7-yl)methyl)carbamoyl)cyclohexane-1-carboxylate, 230 [Step 1]: To a stirred solution of 7-((methylamino)methyl)-4-(o-tolyl)-2H-chromen-2-one (195, 130 mg, 0.5 mmol) and (1S,4S)-4-(methoxycarbonyl)cyclohexane-1-carboxylic acid (104 mg, 0.6 mmol) in DMF (3 mL) was added HATU (195 mg, 0.5 mmol) followed by DIPEA (0.1 mL, 0.7 mmol), and the reaction mixture was stirred at ambient temperature. After 16 h., the reaction mixture was quenched with ice-cold water, and extracted with EtOAc. The combined organic phase was washed with brine, dried over anhydrous Na₂SO₄, filtered, and concentrated under reduced pressure to afford methyl (1S,4S)-4-(methyl((2-oxo-4-(o-tolyl)-2H-chromen-7-yl)methyl)carbamoyl)cyclohexane-1-carboxylate (230, 250 mg). LCMS (ESI) Calcd. for C₂₇H₂₉NO₅: 447, found [M+H]⁺=448.

Synthesis of (1S,4S)-4-(methyl((2-oxo-4-(o-tolyl)-2H-chromen-7-yl)methyl)carbamoyl)cyclohexane-1-carboxylic acid, Example 158 [Step 2]: To a solution of methyl(1S,4S)-4-(methyl((2-oxo-4-(o-tolyl)-2H-chromen-7-yl)methyl)carbamoyl)cyclohexane-1-carboxylate (230, 250 mg, 0.6 mmol) in THF (4 mL)-water (1 mL) mixture was added lithium hydroxide monohydrate (70 mg, 1.7 mmol), and the reaction mixture was stirred at ambient temperature. After 16 h., the reaction mixture was concentrated under reduced pressure. The residue was acidified with 1N aqueous HCl to pH 2-3 and extracted with 20% MeOH-DCM. The combined organic phase was washed with brine, dried over anhydrous Na₂SO₄, filtered, and concentrated under reduced pressure. The product was purified by RP prep-HPLC to afford (1S,4S)-4-(methyl((2-oxo-4-(o-tolyl)-2H-chromen-7-yl)methyl)carbamoyl)cyclohexane-1-carboxylic acid (Example 158, 33 mg, racemic). LCMS (ESI) Calcd. for C₂₆H₂₇NO₅: 433, found [M+H]⁺=434. ¹H NMR (400 MHz, methanol-d₄): δ 7.44-7.34 (m, 3H), 7.26-7.24 (m, 2H), 7.10-7.09 (m, 1H), 6.98-6.97 (m, 1H), 6.29 (s, 1H), 4.63 (s, 2H), 3.00 (s, 3H), 2.72-2.67 (m, 1H), 2.14 (s, 3H), 2.05-2.03 (m, 2H), 1.66-1.58 (m, 6H).

### Example 159-162: Synthesis of ethyl (S)-1-((R)-2-((4-(4-fluorophenyl)-7-methyl-2-oxo-2H-chromen-5-yl)oxy)propanoyl)piperidine-3-carboxylate, (S)-1-((R)-2-((4-(4-fluorophenyl)-7-methyl-2-oxo-2H-chromen-5-yl)oxy)propanoyl)piperidine-3-carboxylic acid, (R)-4-(4-fluorophenyl)-7-methyl-5-((1-oxo-1-(piperidin-1-yl)propan-2-yl)oxy)-2H-chromen-2-one, (R)-2-((4-(4-fluorophenyl)-7-methyl-2-oxo-2H-chromen-5-yl)oxy)-N,N-dimethylpropanamide

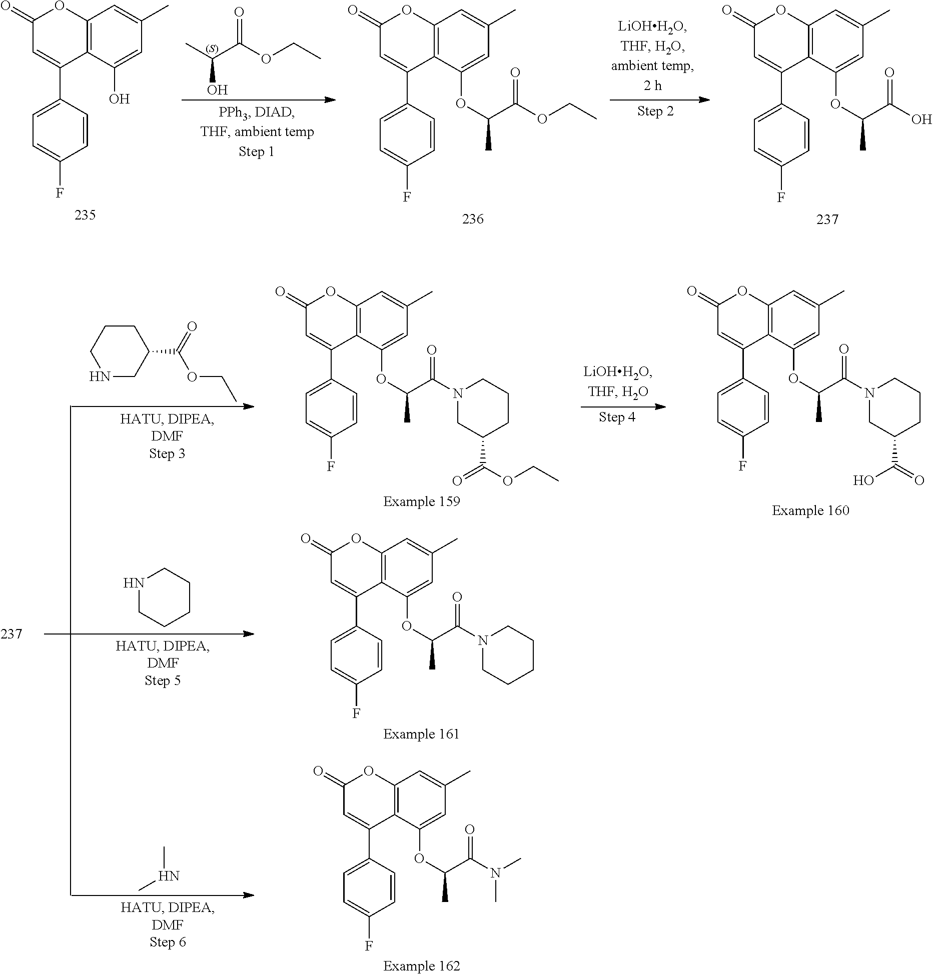

Synthesis of ethyl (R)-2-((4-(4-fluorophenyl)-7-methyl-2-oxo-2H-chromen-5-yl)oxy)propanoate, 236 [Step 1]: To a stirred solution of 4-(4-fluorophenyl)-5-hydroxy-7-methyl-chromen-2-one (500 mg, 1.8 mmol) in THF (5 mL) was added ethyl (S)-2-hydroxypropanoate (240 mg, 2 mmol) followed by triphenylphosphine (1.46 g, 5.5 mmol). 4A Molecular sieves (˜300 mg) were added to the solution. DIAD (1.1 mL, 5.5 mmol) was added to the solution at 0° C. and stirred at ambient temperature for 16 h. The reaction mass was filtered, and the filtrate liquid was concentrated under reduced pressure. The product was partitioned between EtOAc and water. The organic phase was washed with brine, dried over anhydrous Na₂SO₄, and purified by flash column chromatography to afford ethyl (R)-2-((4-(4-fluorophenyl)-7-methyl-2-oxo-2H-chromen-5-yl)oxy)propanoate (236, 500 mg). LCMS (ESI) Calcd. for C₂₁H₁₉FO₅: 370, found [M+H]⁺=371. ¹H NMR (400 MHz, DMSO-d₆) δ 7.38 (br s, 2H), 7.24-7.19 (m, 2H), 6.92 (s, 1H), 6.52 (s, 1H), 6.08 (s, 1H), 4.84 (br s, 1H), 4.07 (br s, 2H), 2.34 (s, 3H), 1.10-1.14 (m, 3H), 0.84 (d, 3H).

Synthesis of (R)-2-((4-(4-fluorophenyl)-7-methyl-2-oxo-2H-chromen-5-yl)oxy)propanoic acid, 237 [Step 2]: To a solution of ethyl (R)-2-((4-(4-fluorophenyl)-7-methyl-2-oxo-2H-chromen-5-yl)oxy)propanoate (236, 500 mg, 1.4 mmol) in THF (5 mL) was added a solution of lithium hydroxide monohydrate (170 mg, 4 mmol) in water (2 mL) and stirred at ambient temperature overnight. Solvent was evaporated under reduced pressure. The product was acidified using 1N HCl to pH 5 and extracted with EtOAc. Combined organic layer was washed with brine, dried over anhydrous Na₂SO₄. and concentrated under reduced pressure. The mixture was triturated with n-pentane and dried under reduced pressure to afford (R)-2-((4-(4-fluorophenyl)-7-methyl-2-oxo-2H-chromen-5-yl)oxy)propanoic acid (237, 300 mg). LCMS (ESI) Calcd. for C₁₉H₁₅FO₅: 342; [M+H]⁺=343. ¹H NMR (400 MHz, DMSO-d₆) δ 13.01 (br s, 1H), 7.39 (br s, 2H), 7.23-7.19 (m, 2H), 6.90 (s, 1H), 6.51 (s, 1H), 6.07 (s, 1H), 4.75-4.69 (m, 1H), 2.34 (s, 3H), 0.84 (d, 3H).

Synthesis of ethyl (S)-1-((R)-2-((4-(4-fluorophenyl)-7-methyl-2-oxo-2H-chromen-5-yl)oxy)propanoyl)piperidine-3-carboxylate, Example 159 [Step 3]: To a reaction vessel purged and maintained under nitrogen was added (R)-2-((4-(4-fluorophenyl)-7-methyl-2-oxo-2H-chromen-5-yl)oxy)propanoic acid (237, 150 mg, 0.4 mmol) in dichloromethane (5 mL) was added DIPEA (0.25 mL, 1.3 mmol) followed by HATU (250 mg, 0.65 mmol) at 0° C. Ethyl(S)-piperidine-3-carboxylate (85 mg, 0.5 mmol) was added to it and stirred for 3 h. at ambient temperature. The reaction mixture was partitioned between ethyl acetate and water. Organic layer was collected and washed with brine, dried over anhydrous Na₂SO₄. and evaporated under reduced pressure. The product was purified via prep-HPLC and lyophilized to afford ethyl (S)-1-((R)-2-((4-(4-fluorophenyl)-7-methyl-2-oxo-2H-chromen-5-yl)oxy)propanoyl)piperidine-3-carboxylate (Example 159, 125 mg). LCMS (ESI) Calcd. for C₂₇H₂₈FNO₆: 481, found [M+H]⁺=482. ¹H NMR (400 MHz, DMSO-d₆ at 100° C.) δ 7.41-7.37 (m, 2H), 7.18-7.14 (m, 2H), 6.89 (s, 1H), 6.49 (s, 1H), 6.02 (s, 1H), 5.06-5.01 (m, 1H), 4.11-4.03 (q, 2H), 3.50 (br s, 1H), 3.05 (br s, 1H), 2.98 (br s, 1H), 2.30-2.37 (s, 3H), 1.89 (br s, 1H), 1.69-1.59 (m, 2H), 1.38 (br s, 1H), 1.27 (t, 3H), 0.90 (d, 3H).

Synthesis of (S)-1-((R)-2-((4-(4-fluorophenyl)-7-methyl-2-oxo-2H-chromen-5-yl)oxy)propanoyl)piperidine-3-carboxylic acid, Example 160 [Step 4]: To a solution of ethyl (S)-1-((R)-2-((4-(4-fluorophenyl)-7-methyl-2-oxo-2H-chromen-5-yl)oxy)propanoyl)piperidine-3-carboxylate (Example 159, 100 mg, 0.2 mmol) in water (1 mL) and THF (3 mL) was added lithium hydroxide monohydrate (25 mg, 0.6 mmol) and stirred at ambient temperature for 2 h. The resulting mixture was concentrated under reduced pressure, diluted with water, and extracted with EtOAc. The aqueous layer was acidified with 1N HCl to pH 5 and extracted with EtOAc. Combined organic layer was washed with brine, dried over anhydrous Na₂SO₄, and concentrated under reduced pressure. The product was purified via reverse phase prep-HPLC and lyophilized to afford(S)-1-((R)-2-((4-(4-fluorophenyl)-7-methyl-2-oxo-2H-chromen-5-yl)oxy)propanoyl)piperidine-3-carboxylic acid (Example 160, 45 mg). LCMS (ESI) Calcd. for C₂₅H₂₄FNO₆: 453, found [M+H]⁺=454. ¹H NMR (400 MHz, DMSO-d₆ at 100° C.) δ 12.5 (br s, 1H), 7.42 (br s, 2H), 7.14-7.18 (m, 2H), 6.86 (s, 1H), 6.47 (br s, 1H), 6.01 (s, 1H), 5.04 (br s, 1H), 3.56 (br s, 1H), 2.34 (s, 3H), 2.22 (br s, 1H), 1.90 (br s, 1H), 1.62 (br s, 2H), 1.38 (br s, 1H), 0.87 (d, 3H).

Synthesis of (R)-4-(4-fluorophenyl)-7-methyl-5-((1-oxo-1-(piperidin-1-yl)propan-2-yl)oxy)-2H-chromen-2-one, Example 161 [Step 5]: To a solution of (R)-2-((4-(4-fluorophenyl)-7-methyl-2-oxo-2H-chromen-5-yl)oxy)propanoic acid (237, 100 mg, 0.3 mmol) in dichloromethane (3 mL) was added DIPEA (0.2 mL, 0.7 mmol) followed by HATU (165 mg, 0.4 mmol) at 0° C. Piperidine (30 mg, 0.35 mmol) was added and stirred at ambient temperature for 3 h. The reaction mixture was partitioned between ethyl acetate and water. Organic layer was collected and washed with brine, dried over anhydrous Na₂SO₄, and evaporated under reduced pressure. The product was purified via reverse phase prep-HPLC to afford (R)-4-(4-fluorophenyl)-7-methyl-5-((1-oxo-1-(piperidin-1-yl)propan-2-yl)oxy)-2H-chromen-2-one (Example 161, 56 mg). LCMS (ESI) Calcd. for C₂₄H₂₄FNO₄: 409, found [M+H]⁺=410. ¹H NMR (400 MHz, DMSO-d₆) δ 7.41 (br s, 2H), 7.20 (t, 2H), 6.89 (s, 1H), 6.40 (s, 1H), 6.06 (s, 1H), 5.10-5.05 (m, 1H), 3.43 (br s, 2H), 3.27 (br s, 2H), 2.33 (s, 3H), 1.57 (br s, 2H), 1.43 (br s, 4H), 0.83 (d, 3H).

Synthesis of (R)-2-((4-(4-fluorophenyl)-7-methyl-2-oxo-2H-chromen-5-yl)oxy)-N,N-dimethylpropanamide, Example 162 [Step 6]: To a solution of (R)-2-((4-(4-fluorophenyl)-7-methyl-2-oxo-2H-chromen-5-yl)oxy)propanoic acid (237, 50 mg, 0.1 mmol) in DCM (4 mL) was added N-methylmethanamine (10 mg, 0.22 mmol) followed by HATU (85 mg, 0.2 mmol) and DIPEA (0.05 mL, 0.3 mmol) at 0° C. and stirred at ambient temperature for 6 h. The reaction mass was diluted with DCM and washed with water and brine, dried over anhydrous Na₂SO₄, and concentrated under reduced pressure. The product was purified via reverse phase prep-HPLC and lyophilized to afford (R)-2-((4-(4-fluorophenyl)-7-methyl-2-oxo-2H-chromen-5-yl)oxy)-N,N-dimethylpropanamide (Example 162, 33 mg). LCMS (ESI) Calcd for C₂₁H₂₀FNO₄: 369, found [M+H]⁺=370. ¹H NMR (400 MHz, DMSO d₆) δ 7.40 (br s, 2H), 7.18 (t, 2H), 6.88 (s, 1H), 6.40 (s, 1H), 6.05 (s, 1H), 5.09-5.05 (m, 1H), 2.88 (s, 3H), 2.74 (s, 3H), 2.34 (s, 3H), 0.85 (d, 3H).

### Example 163: Synthesis of 7-(methoxymethyl)-5-methyl-4-(o-tolyl)-2H-chromen-2-one

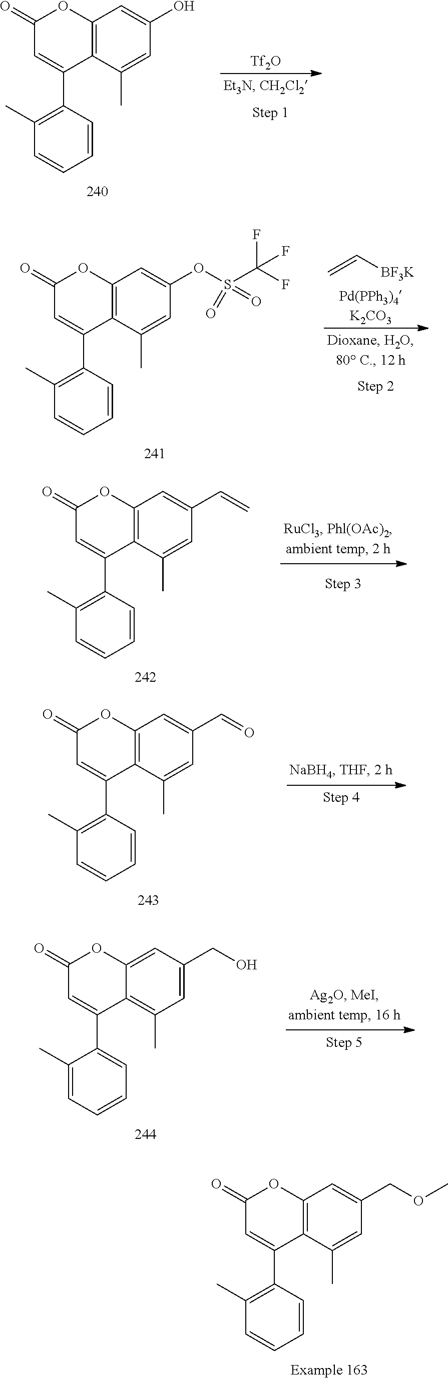

Synthesis of 5-methyl-2-oxo-4-(o-tolyl)-2H-chromen-7-yl trifluoromethanesulfonate 241, [Step 1]: To a solution of 7-hydroxy-5-methyl-4-(o-tolyl)-2H-chromen-2-one (240, 230 mg, 0.9 mmol) in DCM (3 mL) was added triethylamine (0.25 mL, 1.7 mmol) followed by trifluoromethanesulfonic anhydride (0.45 mL, 1.72 mmol) and stirred at ambient temperature for 16 h. The reaction mixture was concentrated under reduced pressure and partitioned between EtOAc and water. Organic layer was collected and washed with brine, dried over anhydrous Na₂SO₄, and concentrated under reduced pressure. The product was purified by flash chromatography to afford 5-methyl-2-oxo-4-(o-tolyl)-2H-chromen-7-yl trifluoromethanesulfonate (241, 230 mg). LCMS (ESI) Calcd for C₁₈H₁₃F₃O₅S: 398, found [M+H]⁺=399. ¹H NMR (400 MHz, DMSO-d₆) δ 7.70 (d, 1H), 7.40-7.38 (m, 1H), 7.35-7.32 (m, 2H), 7.30-7.29 (m, 1H), 7.27 (d, 1H), 6.32 (s, 1H), 2.10 (s, 3H), 1.72 (s, 3H).

Synthesis of 5-methyl-4-(o-tolyl)-7-vinyl-2H-chromen-2-one, 242 [Step 2]: To a solution of [5-methyl-4-(o-tolyl)-2-oxo-chromen-7-yl]trifluoromethanesulfonate (241, 250 mg, 0.6 mmol) in 1,4-dioxane (8 mL) and water (2 mL) was added K₂CO₃ (260 mg, 1.9 mmol) and degassed with argon for 10 min. Potassium vinyltrifluoroborate (110 mg, 0.8 mmol) followed by Pd(PPh₃)₄ (75 mg, 0.1 mmol) was added to it and heated at 100° C. for 12 h. The reaction mixture was filtered through a celite bed, and the filtrate liquid was evaporated and partitioned between EtOAc and water. Organic extract was washed with brine, dried over anhydrous Na₂SO₄, and concentrated under reduced pressure. The product was purified by flash chromatography to afford 5-methyl-4-(o-tolyl)-7-vinyl-chromen-2-one (242, 115 mg). LCMS (ESI) Calcd for C₁₉H₁₆O₂: 276, found [M+H]⁺=277.1H NMR (400 MHz, DMSO-d₆) δ 7.47 (s, 1H), 7.39-7.30 (m, 3H), 7.26 (d, 2H), 6.80-6.73 (dd, 1H), 6.16 (s, 1H), 6.08-6.04 (d, 1H), 5.47-5.44 (d, 1H), 2.08 (s, 3H), 1.68 (s, 3H).

Synthesis of 5-methyl-2-oxo-4-(o-tolyl)-2H-chromene-7-carbaldehyde, 243 [Step 3]: To a stirred solution of 5-methyl-4-(o-tolyl)-7-vinyl-chromen-2-one (242, 120 mg, 0.4 mmol) in dichloromethane (4 mL) and H₂O (1 mL) was added RuCl₃ (9.0 mg, 0.1 mmol) followed by PhI(OAc)₂ (420 mg, 1.3 mmol) and stirred at ambient temperature for 2 h. The reaction mixture was filtered through a celite bed. Filtrate liquid was washed with water, brine, dried over anhydrous Na₂SO₄, and concentrated. The product was purified via flash chromatography to afford 5-methyl-4-(o-tolyl)-2-oxo-chromene-7-carbaldehyde (243, 50 mg). LCMS (ESI) Calcd. for C₁₈H₁₄O₃: 278, found [M+H]⁺=279. ¹H NMR (400 MHz, DMSO-d₆) δ 10.06 (s, 1H), 7.84 (s, 1H), 7.59 (s, 1H), 7.43 (t, 1H), 7.36 (t, 2H), 7.28 (d, 1H), 6.39 (s, 1H), 2.09 (s, 3H), 1.76 (s, 3H).

Synthesis of 7-(hydroxymethyl)-5-methyl-4-(o-tolyl)-2H-chromen-2-one, 244 [Step 4]: To a stirred solution of 5-methyl-4-(o-tolyl)-2-oxo-chromene-7-carbaldehyde (243, 50 mg, 0.2 mmol) in THF (3 mL) was added NaBH₄ (10 mg, 0.3 mmol) at 0° C. and stirred at ambient temperature for 2 h. The reaction mixture was quenched with water and extracted with EtOAc. Organic extract was washed with brine, dried over anhydrous Na₂SO₄, and concentrated under reduced pressure. The product was purified by flash chromatography to afford 7-(hydroxymethyl)-5-methyl-4-(o-tolyl)-2H-chromen-2-one (244, 50 mg). LCMS (ESI) Calcd. for C₁₈H₁₆O₃: 280, found [M+H]⁺=281. ¹H NMR (400 MHz, DMSO-d₆) δ 7.41-7.37 (m, 1H), 7.34-7.30 (m, 2H), 7.26-7.23 (m, 2H), 7.03 (s, 1H), 6.14 (s, 1H), 5.41 (t, 1H), 4.56 (d, 2H), 2.07 (s, 3H), 1.66 (s, 3H). 0464 Synthesis of 7-(methoxymethyl)-5-methyl-4-(o-tolyl)-2H-chromen-2-one, Example 163 [Step 5]: To a mixture of 7-(hydroxymethyl)-5-methyl-4-(o-tolyl)-2H-chromen-2-one (244, 50 mg, 0.2 mmol) and methyl iodide (0.3 mL, 5.3 mmol) was added Ag₂O (85 mg, 0.4 mmol) and stirred at ambient temperature for 16 h. The reaction mixture was filtered through celite bed and washed with EtOAc. It was washed with saturated aqueous NH₄Cl solution, brine, dried over anhydrous Na₂SO₄, and concentrated under reduced pressure. The product was purified by flash chromatography and then via reverse phase prep-HPLC and lyophilized to afford 7-(methoxymethyl)-5-methyl-4-(o-tolyl)-2H-chromen-2-one (Example 163, 18 mg). LCMS (ESI) Calcd. for C₁₉H₁₈O₃: 294, found [M+H]⁺=295. ¹H NMR (400 MHz, DMSO-d₆) δ 7.39 (t, 1H), 7.34 (d, 2H), 7.27 (d, 2H), 7.03 (s, 1H), 6.17 (s, 1H), 4.47 (s, 2H), 3.29 (s, 3H), 2.08 (s, 3H), 1.67 (s, 3H).

### Example 164: Synthesis of 7-((pyridin-2-ylmethyl)amino)-4-(o-tolyl)-2H-chromen-2-one

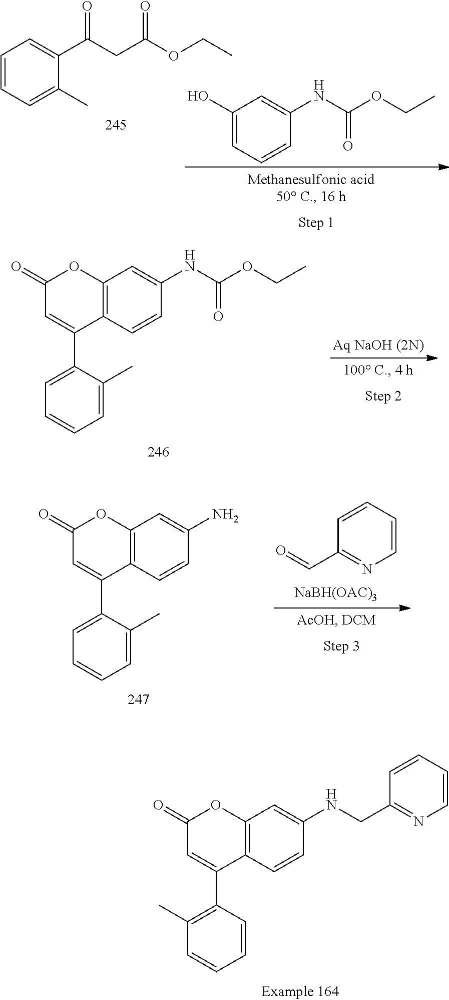 [SMILES: CCOC(=O)CC(=O)c1ccccc1C ; CCOC(=O)Nc1cccc(O)c1 ; CCOC(=O)Nc1ccc2c(-c3ccccc3C)cc(=O)oc2c1 ; Cc1ccccc1-c1cc(=O)oc2cc(N)ccc12 ; O=Cc1ccccn1 ; Cc1ccccc1-c1cc(=O)oc2cc(NCc3ccccn3)ccc12]

Synthesis of ethyl (2-oxo-4-(o-tolyl)-2H-chromen-7-yl)carbamate, 246 [Step 1]: In methanesulphonic acid (30 mL, 23.6 mmol), both ethyl 3-oxo-3-(o-tolyl)propanoate (245, 6.6 g, 30.8 mmol) and ethyl (3-hydroxyphenyl)carbamate (4.8 g, 26.5 mmol) were mixed under N₂-atmosphere at ambient temperature and stirred at 50° C. for 16 h. The reaction was quenched with ice cold water with vigorous stirring. The reaction was diluted in ethyl acetate and washed with water. Organic extract was dried over anhydrous Na₂SO₄ and concentrated under reduced pressure. The product was purified by column chromatography followed by trituration with diethyl ether and pentane mixture to afford ethyl (2-oxo-4-(o-tolyl)-2H-chromen-7-yl)carbamate (246, 2.2 g). LCMS (ESI) Calcd. for C₁₉H₁₇NO₄: 323, found [M+H]⁺=324. ¹H NMR (400 MHz, DMSO-d₆) δ 10.17 (s, 1H), 7.64 (d, 1H), 7.42-7.38 (m, 2H), 7.34-7.30 (m, 2H), 7.24-7.23 (d, 1H), 6.90-6.87 (d, 1H), 6.19 (s, 1H), 4.19-4.13 (q, 2H), 2.11 (s, 3H), 1.27-1.23 (t, 3H).

Synthesis of 7-amino-4-(o-tolyl)-2H-chromen-2-one, 247 [Step 2]: To the stirred solution of ethyl (2-oxo-4-(o-tolyl)-2H-chromen-7-yl)carbamate (246, 3.0 g, 9.3 mmol) in THF (20 mL), was added NaOH (50 mL, 2N) dropwise and stirred at 100° C. for 4 h. The reaction mixture was diluted with water and washed with diethyl ether. The aqueous layer was neutralized by 0.5N HCl to maintain pH 8 at 0° C. The aqueous layer was concentrated and the residue was extracted with 20% IPA in CHCl₃ solution. The organic layer was dried over anhydrous Na₂SO₄ and concentrated under reduced pressure to obtain the product. The product was purified with a polar silica gel column. The product was triturated using diethyl ether and n-pentane to afford 7-amino-4-(o-tolyl)-2H-chromen-2-one (247, 1.60 g). LCMS (ESI) Calcd. for C₁₆H₁₃NO₂: 251, found [M+H]⁺=252. ¹H NMR (400 MHz, DMSO-d₆) δ 7.41-7.29 (m, 3H), 7.20-7.18 (d, 1H), 6.62-6.60 (d, 1H), 6.48-6.44 (m, 2H), 6.22 (s, 2H), 5.82 (s, 1H), 2.17 (s, 3H).

Synthesis of 7-((pyridin-2-ylmethyl)amino)-4-(o-tolyl)-2H-chromen-2-one, Example 164 [Step 3]: To a solution of 7-amino-4-(o-tolyl)-2H-chromen-2-one (247, 250 mg, 1 mmol) in DCM (2 mL) was added 2-picoline aldehyde (160 mg, 1.5 mmol) followed by AcOH (catalytic amount) at ambient temperature and stirred at 40° C. for 16 h. NaBH(OAc)₃ (845 mg, 4 mmol) was added and the reaction mixture was stirred at ambient temperature for 4 h., and concentrated under reduced pressure. The product was purified by reverse phase prep-HPLC and lyophilized to afford 7-((pyridin-2-ylmethyl)amino)-4-(o-tolyl)-2H-chromen-2-one (Example 164, 25 mg). LCMS (ESI) Calcd. for C₂₂H₁₈N₂O₂: 342, found [M+H]⁺=343. ¹H NMR (400 MHz, DMSO-d₆) δ 8.55 (d, 1H), 7.78-7.75 (m, 1H), 7.44-7.43 (m, 1H), 7.39-7.26 (m, 5H), 7.19 (d, 1H), 6.65-6.57 (m, 2H), 6.49 (br s, 1H), 5.48 (s, 1H), 4.46 (d, 2H), 2.09 (s, 3H).

### Example 165: Synthesis of 7-((thiazol-2-ylmethyl)amino)-4-(o-tolyl)-2H-chromen-2-one

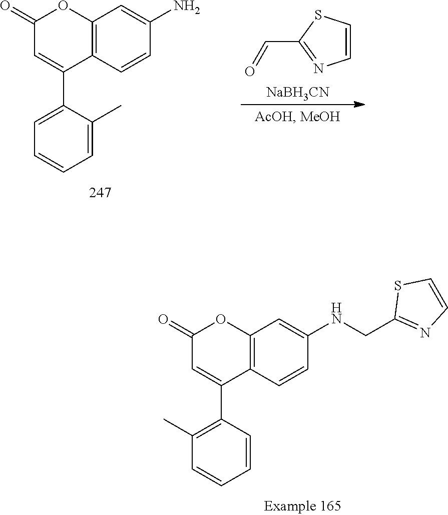

Synthesis of 7-((thiazol-2-ylmethyl)amino)-4-(o-tolyl)-2H-chromen-2-one, Example 165: To a solution of 7-amino-4-(o-tolyl)-2H-chromen-2-one (247, 120 mg, 0.5 mmol) in methanol (2 mL) was added thiazole-2-carbaldehyde (2, 80 mg, 0.7 mmol) followed by AcOH (catalytic amount) at ambient temperature and stirred for 16 h. at 60° C. After that sodium cyanoborohydride (45 mg, 0.72 mmol) was added and continue stirring at ambient temperature for 4 h. The reaction mixture was concentrated under reduced pressure, diluted with EtOAc, washed with water and brine, dried over anhydrous sodium sulfate, and concentrated under reduced pressure. The product was purified by reverse phase prep-HPLC and lyophilized to afford 7-((thiazol-2-ylmethyl)amino)-4-(o-tolyl)-2H-chromen-2-one (Example 165, 26 mg). LCMS (ESI) Calcd. for C₂₀H₁₆N₂O₂S: 348, found [M+H]⁺=349. ¹H NMR (400 MHz, DMSO-d₆) δ 7.77-7.76 (m, 1H), 7.63-7.59 (m, 2H), 7.41-7.31 (m, 3H), 7.20-7.18 (m, 1H), 6.68-6.66 (m, 1H), 6.61 (d, 1H), 5.59-5.56 (m, 1H), 5.89 (s, 1H), 4.72-4.70 (m, 2H), 2.10 (s, 3H).

### Example 166: Synthesis of 7-((oxazol-2-ylmethyl)amino)-4-(o-tolyl)-2H-chromen-2-one

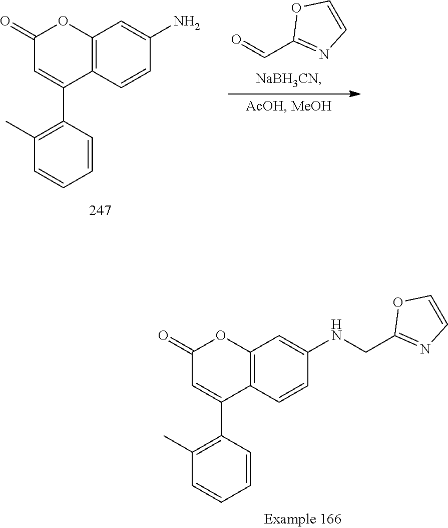 [SMILES: Cc1ccccc1-c1cc(=O)oc2cc(N)ccc12 ; O=Cc1ncco1 ; Cc1ccccc1-c1cc(=O)oc2cc(NCc3ncco3)ccc12]

Synthesis of 7-((oxazol-2-ylmethyl)amino)-4-(o-tolyl)-2H-chromen-2-one, Example 166: To a solution of 7-amino-4-(o-tolyl)-2H-chromen-2-one (247, 250 mg, 1 mmol) in methanol (2 mL) was added oxazole-2-carbaldehyde (145 mg, 1.5 mmol) followed by AcOH (catalytic amount) at ambient temperature and stirred for 16 h. at 40° C. After that sodium cyanoborohydride (95 mg, 1.5 mmol) was added and continued stirring at ambient temperature for 4 h. The reaction mixture was concentrated under reduced pressure and diluted with EtOAc and washed with water and brine, dried over anhydrous sodium sulfate, and concentrated under reduced pressure. The product was purified by reverse phase prep-HPLC and lyophilized to afford 7-((oxazol-2-ylmethyl)amino)-4-(o-tolyl)-2H-chromen-2-one (Example 166, 55 mg). LCMS (ESI) Calcd. for C₂₀H₁₆N₂O₃: 332, found [M+H]⁺=333. ¹H NMR (400 MHz, DMSO-d₆) δ 8.70 (s, 1H), 7.41-7.31 (m, 4H), 7.20-7.17 (m, 2H), 6.67-6.65 (m, 1H), 6.60-6.58 (m, 2H), 5.89 (s, 1H), 4.53-4.51 (d, 2H), 2.10 (s, 3H).

### Example 167: Synthesis of 7-(methyl(thiazol-2-ylmethyl)amino)-4-(o-tolyl)-2H-chromen-2-one

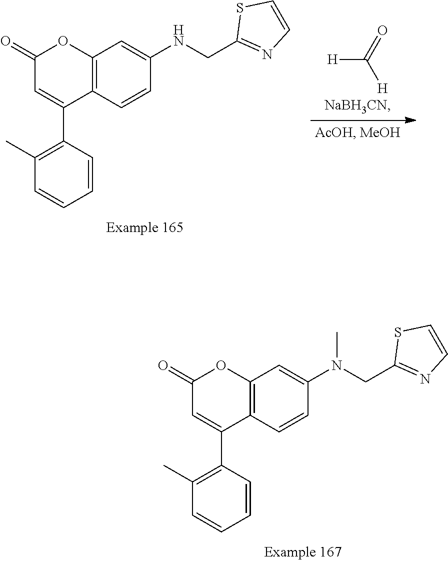 [SMILES: Cc1ccccc1-c1cc(=O)oc2cc(NCc3nccs3)ccc12 ; C=O ; Cc1ccccc1-c1cc(=O)oc2cc(N(C)Cc3nccs3)ccc12]

Synthesis of 7-(methyl(thiazol-2-ylmethyl)amino)-4-(o-tolyl)-2H-chromen-2-one, Example 167: To a solution of 7-((thiazol-2-ylmethyl)amino)-4-(o-tolyl)-2H-chromen-2-one (Example 165, 100 mg, 0.3 mmol) in methanol (2 mL) was added formaldehyde solution (0.1 mL, 0.44 mmol) followed by AcOH (catalytic amount) at ambient temperature and stirred for 16 h. at 60° C. After that sodium cyanoborohydride (27 mg, 0.44 mmol) was added and continue stirred at ambient temperature for 4 h. The reaction mixture was concentrated under reduced pressure and diluted with EtOAc and washed with water and brine, dried over anhydrous sodium sulfate, and concentrated under reduced pressure. The product was purified by reverse phase prep-HPLC and lyophilized to afford 7-(methyl(thiazol-2-ylmethyl)amino)-4-(o-tolyl)-2H-chromen-2-one (Example 167, 15 mg). LCMS (ESI) Calcd. for C₂₁H₁₈N₂O₂S: 362, found [M+H]⁺=363. ¹H NMR (400 MHz, DMSO-d₆): δ 7.77 (d, 1H), 7.64 (d, 1H), 7.40-7.36 (m, 2H), 7.34-7.30 (m, 1H), 7.21-7.19 (m, 1H), 6.78-6.72 (m, 3H), 5.95 (s, 1H), 5.00 (s, 2H), 3.15 (s, 3H), 2.10 (s, 3H).

### Example 168: Synthesis of 7-(methyl(pyridin-2-ylmethyl)amino)-4-(o-tolyl)-2H-chromen-2-one

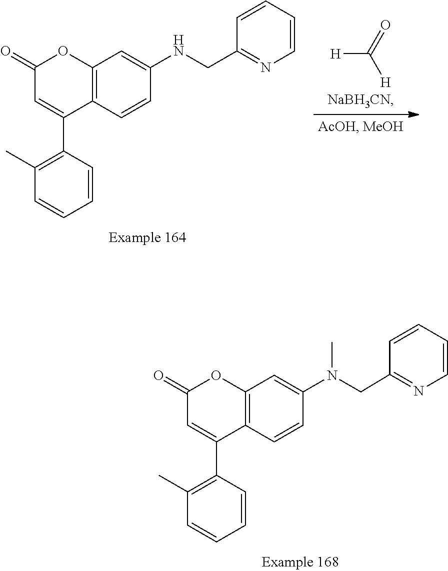

Synthesis of 7-(methyl(pyridin-2-ylmethyl)amino)-4-(o-tolyl)-2H-chromen-2-one, Example 168: To a solution of 7-((pyridin-2-ylmethyl)amino)-4-(o-tolyl)-2H-chromen-2-one (Example 164, 100 mg, 0.3 mmol) in methanol (2 mL) was added formaldehyde solution (0.2 mL, 0.4 mmol) followed by AcOH (catalytic amount) at ambient temperature and stirred for 16 h. at 60° C. After that sodium cyanoborohydride (27 mg, 0.4 mmol) was added and continue stirred at ambient temperature for 4 h. The reaction mixture was concentrated under reduced pressure, diluted with EtOAc, washed with water and brine, dried over anhydrous sodium sulfate, and concentrated under reduced pressure. The product was purified by reverse phase prep-HPLC and lyophilized to afford 7-(methyl(pyridin-2-ylmethyl)amino)-4-(o-tolyl)-2H-chromen-2-one (Example 168, 20 mg). LCMS (ESI) Calcd. for C₂₃H₂₀N₂O₂: 356, found [M+H]⁺=357. ¹H NMR (400 MHz, DMSO-d₆): δ 8.53-8.52 (d, 1H), 7.76-7.71 (t, 1H), 7.41-7.33 (m, 2H), 7.31 (m, 1H), 7.29-7.27 (m, 1H), 7.24-7.18 (t, 2H), 6.71-6.66 (m, 3H), 5.89 (s, 1H), 4.75 (s, 2H), 3.19 (s, 3H), 2.10 (s, 3H).

### Example 169: Synthesis of 7-(((1H-pyrazol-5-yl)methyl)amino)-4-(o-tolyl)-2H-chromen-2-one

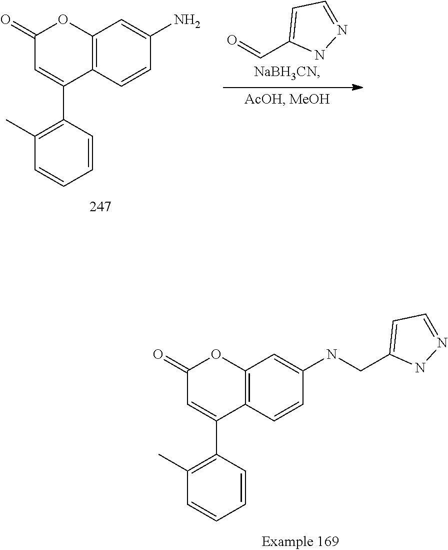 [SMILES: Cc1ccccc1-c1cc(=O)oc2cc(N)ccc12 ; O=Cc1ccn[nH]1 ; Cc1ccccc1-c1cc(=O)oc2cc(NCc3ccn[nH]3)ccc12]

Synthesis of 7-(((1H-pyrazol-5-yl)methyl)amino)-4-(o-tolyl)-2H-chromen-2-one, Example 169: To a solution of 7-amino-4-(o-tolyl)-2H-chromen-2-one (247, 100 mg, 0.4 mmol) in methanol (2 mL) was added 1H-pyrazole-5-carbaldehyde (55 mg, 0.6 mmol) followed by AcOH (catalytic amount) at ambient temperature and stirred for 16 h. at 60° C. After that sodium cyanoborohydride (100 mg, 1.6 mmol) was added and stirring was continued at ambient temperature for 4 h. The reaction mixture was concentrated under reduced pressure, diluted with EtOAc, and washed with water and brine. The organic phase was dried over anhydrous sodium sulfate and concentrated under reduced pressure. The product was purified by reverse phase prep-HPLC and lyophilized to afford 7-(((1H-pyrazol-5-yl)methyl)amino)-4-(o-tolyl)-2H-chromen-2-one (Example 169, 65 mg). LCMS (ESI) Calcd. for C₂₀H₁₇N₃O₂: 331, found [M+H]⁺=332. ¹H NMR (400 MHz, DMSO-d₆): δ 12.65 (br s, 1H), 7.65 (s, 1H), 7.41-7.31 (m, 2H), 7.29 (s, 1H), 7.19-7.15 (m, 2H), 6.63-6.57 (m, 3H), 6.18 (s, 1H), 5.83 (s, 1H), 4.29-4.28 (s, 2H), 2.10 (s, 3H).

### Example 170-171: Synthesis of chiral 7-(3-(dimethylamino)piperidine-1-carbonyl)-4-(o-tolyl)-2H-chromen-2-one

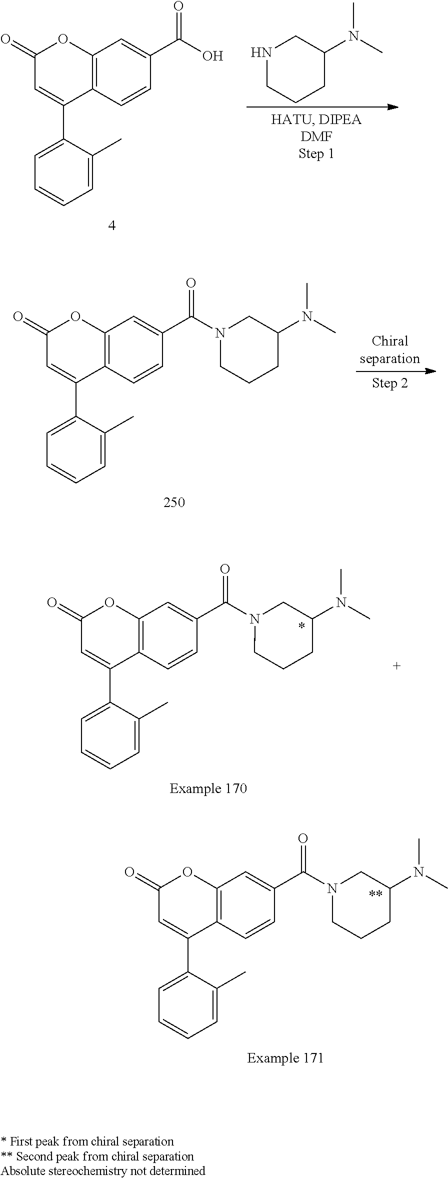

Synthesis of 7-(3-(dimethylamino)piperidine-1-carbonyl)-4-(o-tolyl)-2H-chromen-2-one, 250 [Step 1]: To a stirred solution of 2-oxo-4-(o-tolyl)-2H-chromene-7-carboxylic acid (4, 250 mg, 0.9 mmol) in DMF (2 mL), HATU (510 mg, 1.3 mmol) and N,N-diisopropylethylamine (0.5 mL, 2.7 mmol) were added at ice cold condition and the reaction mixture was stirred for 30 min. Then N,N-dimethylpiperidin-3-amine (230 mg, 1.8 mmol) was added and the reaction mixture was stirred at ambient temperature for 16 h. TLC and LCMS showed full conversion of starting material with formation of new polar spot. The reaction mixture was filtered and the residue was purified by reverse phase prep-HPLC to yield 7-(3-(dimethylamino)piperidine-1-carbonyl)-4-(o-tolyl)-2H-chromen-2-one (250, 230 mg). LCMS (ESI) Calcd for C₂₄H₂₆N₂O₃: 390, found [M+H]⁺=391.

Synthesis of chiral 7-(3-(dimethylamino)piperidine-1-carbonyl)-4-(o-tolyl)-2H-chromen-2-one, Example 170, and 7-(3-(dimethylamino)piperidine-1-carbonyl)-4-(o-tolyl)-2H-chromen-2-one, Example 171 [Step 2]: The racemic compound 7-(3-(dimethylamino)piperidine-1-carbonyl)-4-(o-tolyl)-2H-chromen-2-one (250, 200 mg) was submitted for NP chiral prep purification. The fractions obtained are lyophilized to yield first product as Peak 1, 7-(3-(dimethylamino)piperidine-1-carbonyl)-4-(o-tolyl)-2H-chromen-2-one (Example 170, 70 mg) and Peak 2 as 7-(3-(dimethylamino)piperidine-1-carbonyl)-4-(o-tolyl)-2H-chromen-2-one (Example 171, 60 mg). The absolute stereochemistry of these Examples was not determined.

Example 170: 7-(3-(dimethylamino)piperidine-1-carbonyl)-4-(o-tolyl)-2H-chromen-2-one, Peak 1: LCMS (ESI) Calcd. for C₂₄H₂₆N₂O₃: 390, found [M+H]⁺=391. ¹H NMR (400 MHz, DMSO-d₆ at 100° C.) δ 7.47-7.35 (m, 4H), 7.29-7.24 (m, 2H), 7.04 (d, 1H), 6.39 (s, 1H), 4.25-3.76 (m, 1H), 3.52-3.46 (m, 1H), 2.94 (br s, 2H), 2.33-2.15 (m, 10H), 1.90 (br s, 1H), 1.73 (d, 1H), 1.53-1.41 (m, 2H).

Example 171: 7-(3-(dimethylamino)piperidine-1-carbonyl)-4-(o-tolyl)-2H-chromen-2-one, Peak 2: LCMS (ESI) Calcd. for C₂₄H₂₆N₂O₃: 390, found [M+H]⁺=391. ¹H NMR (400 MHz, DMSO-d₆ at 100° C.) δ 7.47-7.35 (m, 4H), 7.29-7.24 (m, 2H), 7.04 (d, 1H), 6.39 (s, 1H), 4.25-3.76 (m, 1H), 3.52-3.46 (m, 1H), 2.94 (br s, 2H), 2.33-2.15 (m, 10H), 1.90 (br s, 1H), 1.73 (d, 1H), 1.53-1.41 (m, 2H).

Chiral reverse phase prep-HPLC method: Chiral separation was performed on an Agilent 1200 series instrument. Column was a CHIRALPAK IG (250×21 mm) 5μ. Operating at ambient temperature and flow rate is 21.0 ml/min. Mobile phase was the mixture of 70% Hexane, 15% Dichloromethane and 15% Ethanol held this isocratic mixture run for up to 30 min. with detection at 260 nm wavelength.

### Examples 172-173: Synthesis of (R)—N-methyl-N-(1-(2-oxo-4-(o-tolyl)-2H-chromene-7-carbonyl)piperidin-3-yl)acetamide, (S)—N-methyl-N-(1-(2-oxo-4-(o-tolyl)-2H-chromene-7-carbonyl)piperidin-3-yl)acetamide

### General Reaction Scheme:

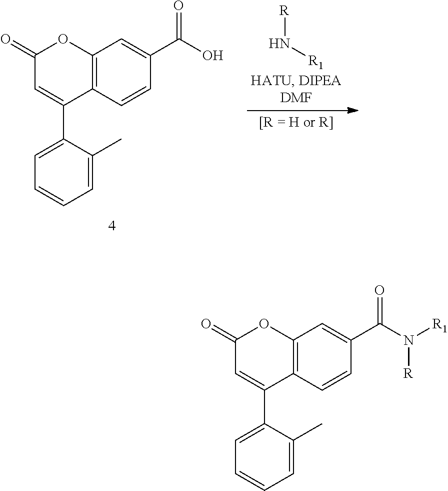

| RNH₂ | Example |
|---|---|
| 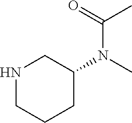 | 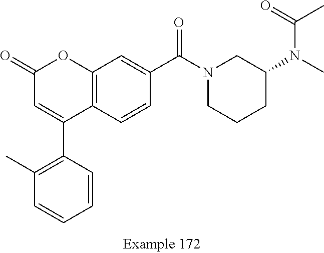 [SMILES: CC(=O)N(C)[C@@H]1CCCN(C(=O)c2ccc3c(-c4ccccc4C)cc(=O)oc3c2)C1] |
| 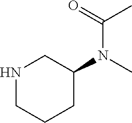 | 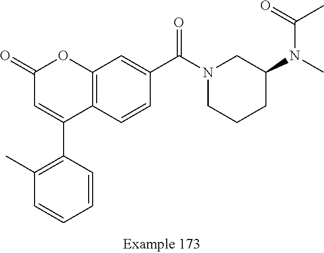 [SMILES: CC(=O)N(C)[C@H]1CCCN(C(=O)c2ccc3c(-c4ccccc4C)cc(=O)oc3c2)C1] |

General procedure for acid amine coupling: To a solution of HATU (0.3 mmol) in DMF (1 mL), 2-oxo-4-(o-tolyl)-2H-chromene-7-carboxylic acid (4, 50 mg, 0.2 mmol) was added followed by N,N-diisopropylethylamine (0.5 mmol). The reaction mixture was stirred for 15 minutes at room temperature under nitrogen balloon. The corresponding amine (0.4 mmol) was added to the reaction mixture and the solution was stirred for 16 h. at ambient temperature. The reaction mixture was then diluted with ethyl acetate and washed with 5% aqueous K₂CO₃ solution. The organic phase was then washed with ice-cold water and brine. It was dried over anhydrous Na₂SO₄, filtered, and evaporated. The product was purified through reverse phase prep-HPLC and lyophilized to afford corresponding product.

Example 172: (R)—N-methyl-N-(1-(2-oxo-4-(o-tolyl)-2H-chromene-7-carbonyl)piperidin-3-yl)acetamide: LCMS (ESI) Calcd. for C₂₅H₂₆N₂O₄: 418, found [M+H]⁺=419. ¹H NMR (400 MHz, DMSO-d₆ at 100° C.) δ 7.47-7.35 (m, 4H), 7.28 (d, 2H), 7.05 (d, 1H), 6.39 (s, 1H), 3.95 (br s, 3H), 3.00 (s, 1H), 2.88-2.82 (m, 4H), 2.16 (s, 3H), 1.98 (s, 3H), 1.84-1.75 (m, 3H), 1.58 (br s, 1H).

Example 173: (S)—N-methyl-N-(1-(2-oxo-4-(o-tolyl)-2H-chromene-7-carbonyl)piperidin-3-yl)acetamide: LCMS (ESI) Calcd. for C₂₅H₂₆N₂O₄: 418, found [M+H]⁺=419. ¹H NMR (400 MHz, DMSO-d₆ at 100° C.) δ 7.47-7.35 (m, 4H), 7.28 (d, 2H), 7.05 (d, 1H), 6.39 (s, 1H), 3.95 (br s, 3H), 3.00 (s, 1H), 2.88-2.82 (m, 4H), 2.16 (s, 3H), 1.98 (s, 3H), 1.84-1.75 (m, 3H), 1.58 (br s, 1H).

### Example 174: Synthesis of (R)—N-((1-(2-oxo-4-(o-tolyl)-2H-chromene-7-carbonyl)pyrrolidin-3-yl)methyl)acetamide

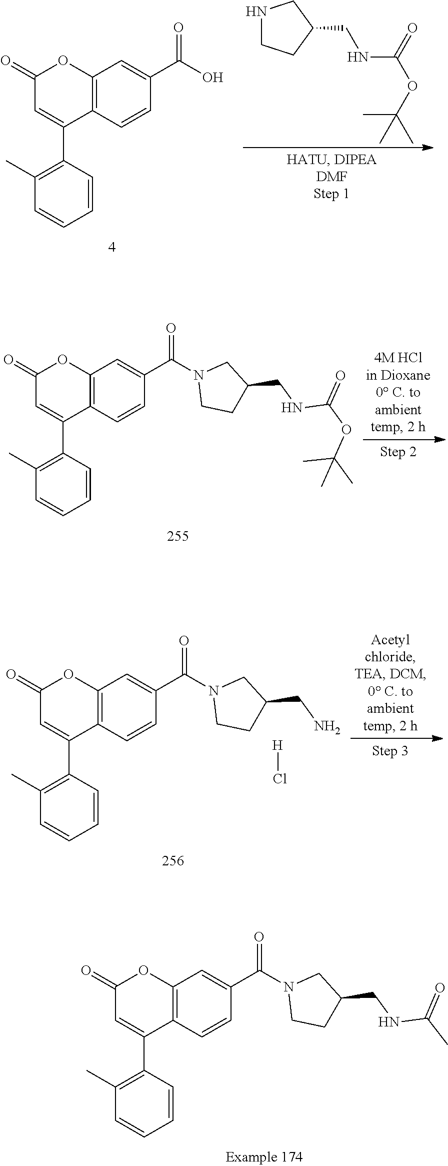

Synthesis of tert-butyl (R)-((1-(2-oxo-4-(o-tolyl)-2H-chromene-7-carbonyl)pyrrolidin-3-yl)methyl)carbamate, 255 [Step 1]: To a stirred solution of 2-oxo-4-(o-tolyl)-2H-chromene-7-carboxylic acid (4, 100 mg, 0.4 mmol) in DMF (1 mL), HATU (205 mg, 0.5 mmol) and N,N-diisopropylethylamine (0.2 mL, 1.1 mmol) were added and the reaction mixture was stirred for 30 min. Then tert-butyl (R)-(pyrrolidin-3-ylmethyl)carbamate (145 mg, 0.7 mmol) and N,N-diisopropylethylamine (0.2 mL, 1.1 mmol) were added and the reaction mixture was stirred at ambient temperature for 16 h. TLC showed full conversion of starting material with formation of new polar spot. The reaction mixture was diluted with EtOAc and washed with water and brine. The organic phase was dried over anhydrous Na₂SO₄, filtered, and evaporated. The product was purified by column chromatography over silica gel to yield tert-butyl (R)-((1-(2-oxo-4-(o-tolyl)-2H-chromene-7-carbonyl)pyrrolidin-3-yl)methyl)carbamate (255, 150 mg). LCMS (ESI) Calcd. for C₂₇H₃₀N₂O₅: 462, found [M+H]⁺=463.

Synthesis of (R)-7-(3-(aminomethyl)pyrrolidine-1-carbonyl)-4-(o-tolyl)-2H-chromen-2-one hydrochloride, 256 [Step 2]: To a stirred solution of tert-butyl (R)-((1-(2-oxo-4-(o-tolyl)-2H-chromene-7-carbonyl)pyrrolidin-3-yl)methyl)carbamate (255, 150 mg, 0.3 mmol) in 1,4-Dioxane (2 mL), 4M HCl (1.0 mL, 3.2 mmol) was added at ice cold condition and the reaction mixture was stirred at ambient temperature for 16 h. TLC showed full conversion of starting material with formation of new polar spot. The reaction mixture was evaporated to yield (R)-7-(3-(aminomethyl)pyrrolidine-1-carbonyl)-4-(o-tolyl)-2H-chromen-2-one hydrochloride (256, 100 mg). LCMS (ESI) Calcd. for C₂₂H₂₂N₂O₃: 362, found [M+H]⁺=363.

Synthesis of (R)—N-((1-(2-oxo-4-(o-tolyl)-2H-chromene-7-carbonyl)pyrrolidin-3-yl)methyl)acetamide, Example 174 [Step 3]: To a stirred solution of (R)-7-(3-(aminomethyl)pyrrolidine-1-carbonyl)-4-(o-tolyl)-2H-chromen-2-one hydrochloride (256, 100 mg, 0.3 mmol) in DCM (5 mL), triethylamine (0.2 mL, 1.4 mmol) was added followed by acetyl chloride (0.06 mL, 0.8 mmol) at 0° C. and stirring was continued for 2 h. The reaction mixture was stirred at ambient temperature for 2 h. TLC showed full conversion of starting material with formation of new polar spot. The reaction mixture was diluted with dichloromethane and washed with water and brine. It was dried over anhydrous Na₂SO₄, filtered and evaporated. The product was purified by reverse phase prep-HPLC and lyophilized to yield (R)—N-((1-(2-oxo-4-(o-tolyl)-2H-chromene-7-carbonyl)pyrrolidin-3-yl)methyl)acetamide (Example 174, 60 mg). LCMS (ESI) Calcd. for C₂₂H₂₄N₂O₄: 404, found [M+H]⁺=405. ¹H NMR (400 MHz, DMSO-d₆ at 100° C.) δ 7.61 (br s, 1H), 7.54 (s, 1H), 7.47-7.35 (m, 4H), 7.27 (d, 1H), 7.05 (d, 1H), 6.39 (s, 1H), 3.53-3.46 (m, 3H), 3.22-3.11 (m, 3H), 2.38-2.33 (m, 1H), 2.16 (s, 3H), 2.00-1.95 (m, 1H), 1.81 (s, 3H), 1.67-1.62 (m, 1H).

### Example 175-176: Synthesis of chiral analogs of N-(1-(2-oxo-4-(o-tolyl)-2H-chromene-7-carbonyl)piperidin-3-yl)acetamide

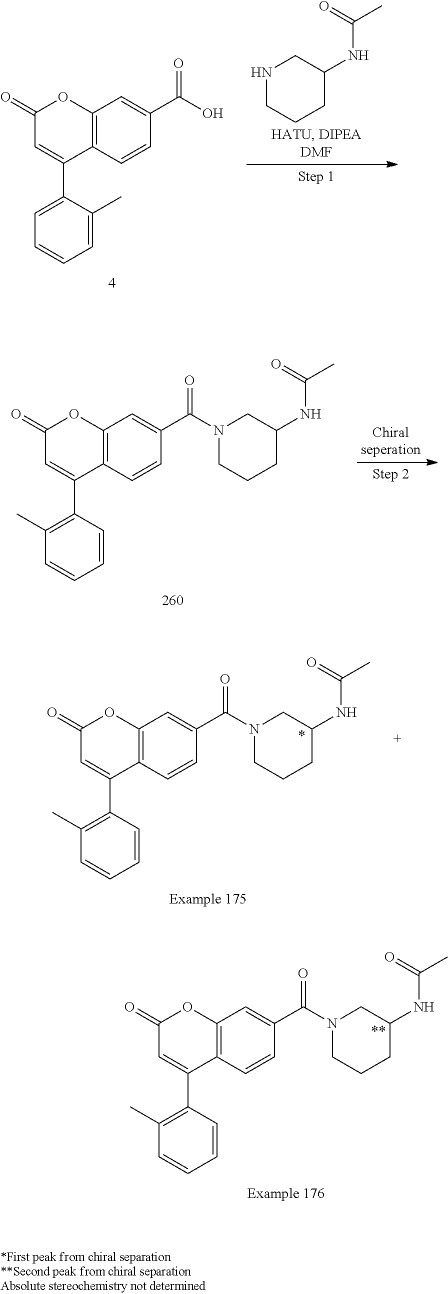

Synthesis of N-(1-(2-oxo-4-(o-tolyl)-2H-chromene-7-carbonyl)piperidin-3-yl)acetamide, 260 [Step 1]: To a solution of 2-oxo-4-(o-tolyl)-2H-chromene-7-carboxylic acid (4, 250 mg, 0.9 mmol) in DMF (1.5 mL), HATU (510 mg, 1.3 mmol) was added followed by addition of N,N-Diisopropylethylamine (0.5 mL, 2.7 mmol). The reaction mixture was stirred for 15 minutes at room temperature under nitrogen balloon. Then N-(piperidin-3-yl)acetamide (190 mg, 1.3 mmol) was added to the reaction mixture and stirred the for 16 h. at ambient temperature. Then the reaction mixture was diluted with EtOAc and washed with 5% aqueous K₂CO₃ solution. The organic phase was then washed with ice cold water and brine. The mixture was dried over anhydrous Na₂SO₄, filtered and evaporated. The product was purified through reverse phase prep-HPLC and lyophilized to afford N-(1-(2-oxo-4-(o-tolyl)-2H-chromene-7-carbonyl)piperidin-3-yl)acetamide (260, 150 mg).

Synthesis of chiral analogs of N-(1-(2-oxo-4-(o-tolyl)-2H-chromene-7-carbonyl)piperidin-3-yl)acetamide, Example 175 and Example 176 [Step 2]: N-(1-(2-oxo-4-(o-tolyl)-2H-chromene-7-carbonyl)piperidin-3-yl)acetamide (260, 150 mg) was purified at ambient temperature via SFC purification and lyophilized to afford Peak 1, N-(1-(2-oxo-4-(o-tolyl)-2H-chromene-7-carbonyl)piperidin-3-yl)acetamide (Example 175, 60 mg) and Peak 2 as N-(1-(2-oxo-4-(o-tolyl)-2H-chromene-7-carbonyl)piperidin-3-yl)acetamide (Example 176, 55 mg). The absolute stereochemistry of these Examples was not determined.

Example 175: N-(1-(2-oxo-4-(o-tolyl)-2H-chromene-7-carbonyl)piperidin-3-yl)acetamide (Peak 1): LCMS (ESI) Calcd. for C₂₄H₂₄N₂O₄: 404; [M+H]⁺=405. ¹H NMR (400 MHz, DMSO-d₆ at 100° C.) δ 7.56 (br s, 1H), 7.46-7.36 (m, 4H), 7.29-7.25 (m, 2H), 7.04 (d, 1H), 6.39 (s, 1H), 3.84-3.47 (m, 4H), 3.24 (br s, 1H), 2.16 (s, 3H), 1.88 (br s, 1H), 1.80 (s, 4H), 1.50 (s, 2H).

Example 176: N-(1-(2-oxo-4-(o-tolyl)-2H-chromene-7-carbonyl)piperidin-3-yl)acetamide (Peak 2): LCMS (ESI) Calcd. For C₂₄H₂₄N₂O₄: 404; [M+H]⁺=405. ¹H NMR (400 MHz, DMSO-d₆ at 100° C.) δ 7.56 (br s, 1H), 7.46-7.36 (m, 4H), 7.29-7.25 (m, 2H), 7.04 (d, 1H), 6.39 (s, 1H), 3.84-3.47 (m, 4H), 3.24 (br s, 1H), 2.16 (s, 3H), 1.88 (br s, 1H), 1.80 (s, 4H), 1.50 (s, 2H).

SFC purification method: Column was an AMYLOSE-SA (20 mm×250 mm), 5μ, with a flow of 60 g/min. and a mobile phase of 75% CO₂ and 25% MeOH with a pressure of 100 bar and a temperature of 35° C. with 214 nm detection.

### Example 177: Synthesis of N-methyl-2-(methyl(2-oxo-4-(o-tolyl)-2H-chromen-7-yl)amino)acetamide

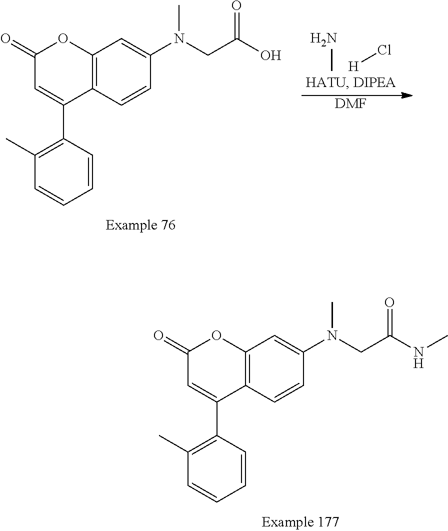

Synthesis of N-methyl-2-(methyl(2-oxo-4-(o-tolyl)-2H-chromen-7-yl)amino)acetamide, Example 177: To a stirred solution of N-methyl-N-(2-oxo-4-(o-tolyl)-2H-chromen-7-yl)glycine (Example 76, 100 mg, 0.3 mmol) and methanamine hydrochloride (45 mg, 0.7 mmol) in DMF (3 mL), was added DIPEA (0.3 mL, 1.9 mmol) followed by HATU (530 mg, 1.4 mmol) at 0° C. and stirred at ambient temperature for 16 h. The reaction mixture was diluted with EtOAc and washed with water and brine, dried over anhydrous sodium sulfate, and concentrated under reduced pressure. The product was purified by reverse phase prep-HPLC and lyophilized to afford N-methyl-2-(methyl(2-oxo-4-(o-tolyl)-2H-chromen-7-yl)amino)acetamide (Example 177, 80 mg). LCMS (ESI) Calcd. for C₂₀H₂₀N₂O₃: 336, found [M+H]⁺=337. ¹H NMR (400 MHz, DMSO-d₆): δ 7.91-7.88 (d, 1H), 7.43-7.30 (m, 3H), 7.21-7.19 (d, 1H), 6.73-6.71 (d, 1H), 6.59-6.56 (m, 2H), 5.92 (s, 1H), 4.01 (s, 2H), 3.06 (s, 3H), 2.59-2.58 (d, 3H), 2.11 (s, 3H).

### Example 178: Synthesis of 7-(((1H-pyrazol-5-yl)methyl)(methyl)amino)-4-(o-tolyl)-2H-chromen-2-one

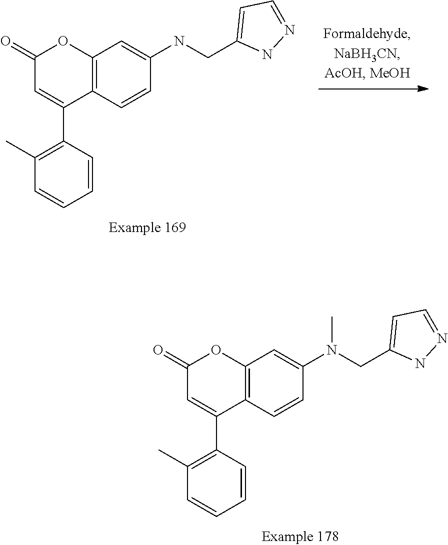 [SMILES: Cc1ccccc1-c1cc(=O)oc2cc(NCc3ccn[nH]3)ccc12 ; Cc1ccccc1-c1cc(=O)oc2cc(N(C)Cc3ccn[nH]3)ccc12]

Synthesis of 7-(((1H-pyrazol-5-yl)methyl)(methyl)amino)-4-(o-tolyl)-2H-chromen-2-one, Example 178: To a solution of 7-(((1H-pyrazol-5-yl)methyl)amino)-4-(o-tolyl)-2H-chromen-2-one (Example 169, 100 mg, 0.3 mmol) in methanol (2 mL) was added formaldehyde (0.1 ml, 0.5 mmol) followed by AcOH (catalytic amount) at ambient temperature and stirred at 60° C. for 16 h. After that sodium cyanoborohydride (30 mg, 0.5 mmol) was added and stirring was continued at ambient temperature for 4 h. The reaction mixture was concentrated under reduced pressure. The residue was diluted with EtOAc and washed with water and brine. The organic phase was dried over anhydrous sodium sulfate and concentrated under reduced pressure. The product was purified by RP prep-HPLC and lyophilized to afford 7-(((1H-pyrazol-5-yl)methyl)(methyl)amino)-4-(o-tolyl)-2H-chromen-2-one (Example 178, 30 mg). LCMS (ESI) Calcd. for C₂₁H₁₉N₃O₂: 345, found [M+H]⁺=346. ¹H NMR (400 MHz, DMSO-d₆): δ 12.7 (s, 1H), 7.62 (s, 1H), 7.42-7.30 (m, 3H), 7.20-7.19 (m, 1H), 6.76-6.69 (m, 3H), 6.12 (m, 1H), 5.89 (s, 1H), 4.58 (s, 2H), 3.09 (s, 3H), 2.10 (s, 3H).

### Example 179: Synthesis of 7-(((1H-tetrazol-5-yl)methyl)(methyl)amino)-4-(o-tolyl)-2H-chromen-2-one

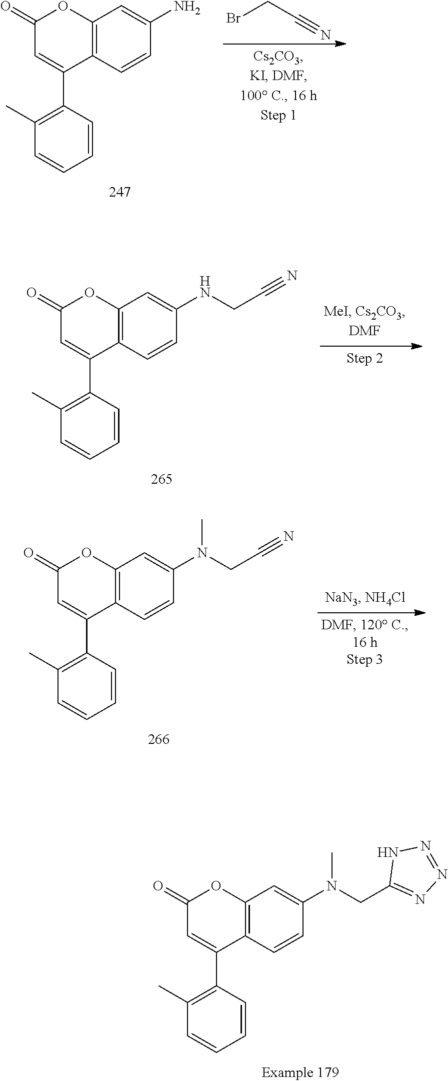

Synthesis of 2-((2-oxo-4-(o-tolyl)-2H-chromen-7-yl)amino) acetonitrile, 265 [Step 1]: A round bottom flask was charged with 7-amino-4-(o-tolyl)-2H-chromen-2-one (247, 200 mg, 0.8 mmol), Cs₂CO₃ (390 mg, 1.2 mmol) and KI (130 mg, 0.8 mmol) in DMF (3 mL) followed by 2-bromoacetonitrile (95 mg, 0.8 mmol) was added and stirred at 100° C. for 16 h. The reaction mixture was diluted with water and extracted with EtOAc. The organic layer was washed with cooled water, brine, dried over anhydrous Na₂SO₄, and concentrated. The product was purified by flash column chromatography by using silica gel to afford 2-((2-oxo-4-(o-tolyl)-2H-chromen-7-yl)amino) acetonitrile (265, 150 mg). LCMS (ESI) Calcd. for C₁₈H₁₄N₂O₂: 290, found [M+H]⁺=291. ¹H NMR (400 MHz, DMSO-d₆) δ 7.41-7.29 (m, 3H), 7.23-7.21 (m, 2H), 6.75 (s, 2H), 6.54 (d, 1H), 5.99 (s, 1H), 4.40 (d, 2H), 2.12 (s, 3H).

Synthesis of 2-(methyl(2-oxo-4-(o-tolyl)-2H-chromen-7-yl)amino) acetonitrile, 266 [Step 2]: A round bottom flask was charged with 2-((2-oxo-4-(o-tolyl)-2H-chromen-7-yl)amino) acetonitrile (265, 130 mg, 0.4 mmol) and Cs₂CO₃ (220 mg, 0.6 mmol) in DMF (3 mL) followed by methyl iodide (75 mg, 0.5 mmol) was added and stirred at ambient temperature for 16 h. The reaction mixture was diluted with water and extracted with EtOAc. The organic layer was washed with cooled water and brine followed by dried over anhydrous Na₂SO₄. and concentrated to afford 2-(methyl(2-oxo-4-(o-tolyl)-2H-chromen-7-yl)amino) acetonitrile (266, 150 mg). LCMS (ESI) Calcd. for C₁₉H₁₆N₂O₂: 304, found, [M+H]⁺=305.

Synthesis of 7-(((1H-tetrazol-5-yl)methyl)(methyl)amino)-4-(o-tolyl)-2H-chromen-2-one, Example 179 [Step 3]: An oven dried sealed tube was charged with 2-(methyl(2-oxo-4-(o-tolyl)-2H-chromen-7-yl)amino) acetonitrile (266, 165 mg, 0.5 mmol), NH₄Cl (265 mg, 4.9 mmol), NaN₃ (210 mg, 0.6 mmol) in DMF (3 mL) and heated at 100° C. for 16 h. The reaction mixture was quenched with saturated NH₄Cl solution and extracted with EtOAc. The combined organic layer was washed with ice cooled water, dried over anhydrous Na₂SO₄, concentrated and purified by reverse phase prep-HPLC to afford 7-(((1H-tetrazol-5-yl)methyl)(methyl)amino)-4-(o-tolyl)-2H-chromen-2-one (Example 179, 14 mg). LCMS (ESI) Calcd. for C₁₉H₁₇N₅O₂: 347, found [M+H]⁺=348. ¹H NMR (400 MHz, DMSO-d₆) δ 7.39-7.35 (m, 2H), 7.31 (t, 3H), 7.19 (d, 1H), 7.06 (br s, 1H), 6.76-6.63 (m, 2H), 6.67 (d, 1H), 5.88 (s, 1H), 3.09 (s, 3H), 2.10 (s, 3H).

### Example 180: Synthesis of (S)-2-(methyl(2-oxo-4-(o-tolyl)-2H-chromen-7-yl)amino)butanoic acid

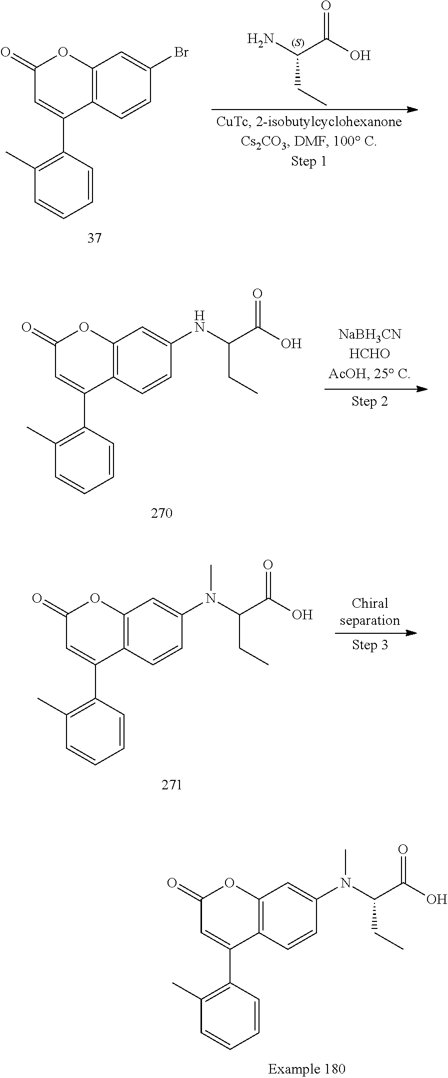

Synthesis of 2-((2-oxo-4-(o-tolyl)-2H-chromen-7-yl)amino)butanoic acid, 270 [Step 1]: A mixture of 7-bromo-4-(o-tolyl) chromen-2-one (37, 300 mg, 1 mmol), (S)-2-aminobutanoic acid (120 mg, 1.1 mmol), copper thiophene 2-carboxylate (35 mg, 0.2 mmol), 2-isobutyrylcyclohexanone (0.1 mL, 0.8 mmol), and in DMF (3 mL) was flushed with argon gas, and the mixture was heated to 100° C. for 12 h. The reaction mixture was diluted with dichloromethane and washed with water, dried over anhydrous Na₂SO₄, and evaporated under reduced pressure. The product was purified by column chromatography to get 2-((2-oxo-4-(o-tolyl)-2H-chromen-7-yl)amino)butanoic acid (270, 200 mg). LCMS (ESI) Calcd. for C₂₀H₁₉NO₄: 337, found [M+H]⁺=338.

Synthesis of 2-(methyl(2-oxo-4-(o-tolyl)-2H-chromen-7-yl)amino)butanoic acid, 271 [Step 2]: To a stirred solution of 2-((2-oxo-4-(o-tolyl)-2H-chromen-7-yl)amino)butanoic acid (270, 200 mg, 0.6 mmol) in acetic acid (1 mL) were added formaldehyde, 37% in aq. solution (0.6 mL, 5.9 mmol) and sodium cyanoborohydride (110 mg, 1.8 mmol) sequentially at 25° C. . . . Then reaction mixture was stirred for 1 h. at ambient temperature. The reaction was quenched with ice water and extracted with 20% MeOH in dichloromethane and washed with brine. The organic phase was dried over anhydrous Na₂SO₄ and evaporated under reduced pressure to get 2-(methyl(2-oxo-4-(o-tolyl)-2H-chromen-7-yl)amino)butanoic acid (271).

Synthesis of (S)-2-(methyl(2-oxo-4-(o-tolyl)-2H-chromen-7-yl)amino)butanoic acid, Example 180 [Step 3]: 2-(methyl(2-oxo-4-(o-tolyl)-2H-chromen-7-yl)amino)butanoic acid (271) product was purified by NP Chiral purification and reverse phase prep-HPLC and lyophilized to afford(S)-2-(methyl(2-oxo-4-(o-tolyl)-2H-chromen-7-yl)amino)butanoic acid (Example 180, 45 mg). LCMS (ESI) Calcd. for C₂₁H₂₁NO₄: 351, found [M+H]⁺=352. ¹H NMR (400 MHz, DMSO-d₆) δ 7.40-7.30 (m, 3H), 7.21 (d, 1H), 6.72-6.66 (m, 3H), 5.85 (s, 1H), 4.20 (s, 1H), 2.85 (s, 3H), 2.12 (s, 3H), 1.95 (d, 1H), 1.72 (d, 1H), 0.83 (t, 3H).

Chiral Prep Method: Chiral separation was performed on an Agilent 1200 series instrument. Column was a CHIRALPAK IG (250×21 mm) 5μ. Operating at ambient temperature and flow rate is 21.0 ml/min. Mobile phase was the mixture of 80% Hexane, 10% Dichloromethane, 10% Ethanol and 0.1% Trifluoroacetic acid held this isocratic mixture run for up to 26 min. with detection at 368 nm wavelength.

### Example 181: Synthesis of 7-(3-((dimethylamino)methyl)pyrrolidine-1-carbonyl)-4-(o-tolyl)-2H-chromen-2-one

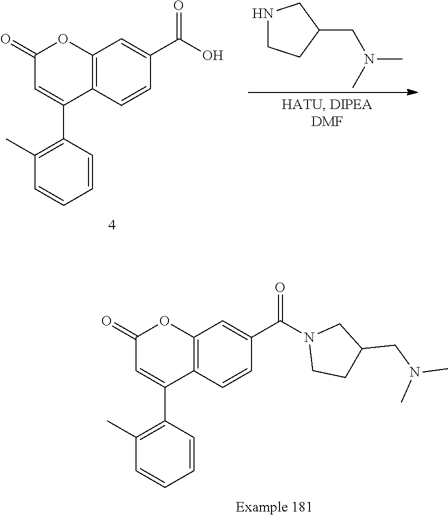

Synthesis of 7-(3-((dimethylamino)methyl)pyrrolidine-1-carbonyl)-4-(o-tolyl)-2H-chromen-2-one, Example 181: To a stirred solution of 2-oxo-4-(o-tolyl)-2H-chromene-7-carboxylic acid (4, 100 mg, 0.4 mmol) in DMF (2 mL), HATU (205 mg, 0.5 mmol) and N,N-Diisopropylethylamine (0.2 mL, 1.1 mmol) were added at ice cold condition and the reaction mixture was stirred for 30 min. Then N,N-dimethyl-1-pyrrolidin-3-yl-methanamine (90 mg, 0.7 mmol) was added and the reaction mixture was stirred at ambient temperature for 16 h. TLC and LCMS showed full conversion of starting material with formation of new polar spot. The reaction mixture was diluted with EtOAc and washed with 5% K₂CO₃, water and brine. The organic phase was dried over anhydrous Na₂SO₄, filtered and evaporated. The product was purified by reverse phase prep-HPLC and lyophilized to yield 7-(3-((dimethylamino)methyl)pyrrolidine-1-carbonyl)-4-(o-tolyl)-2H-chromen-2-one (Example 181, 50 mg). LCMS (ESI) Calcd. for C₂₄H₂₆N₂O₃: 390, found [M+H]⁺=391.2. ¹H NMR (400 MHz, DMSO-d₆) δ 7.54 (br s, 1H), 7.47-7.35 (m, 4H), 7.28 (d, 1H), 7.04 (d, 1H), 6.39 (s, 1H), 3.55-3.47 (m, 3H), 3.20 (br s, 1H), 2.42-2.37 (m, 1H), 2.23 (br s, 2H), 2.16 (br s, 9H), 2.00 (br s, 1H), 1.66-1.59 (m, 1H).

### Example 182-183: Synthesis of methyl 1-(2-oxo-4-(o-tolyl)-2H-chromene-7-carbonyl) azetidine-3-carboxylate, 1-(2-oxo-4-(o-tolyl)-2H-chromene-7-carbonyl) azetidine-3-carboxamide

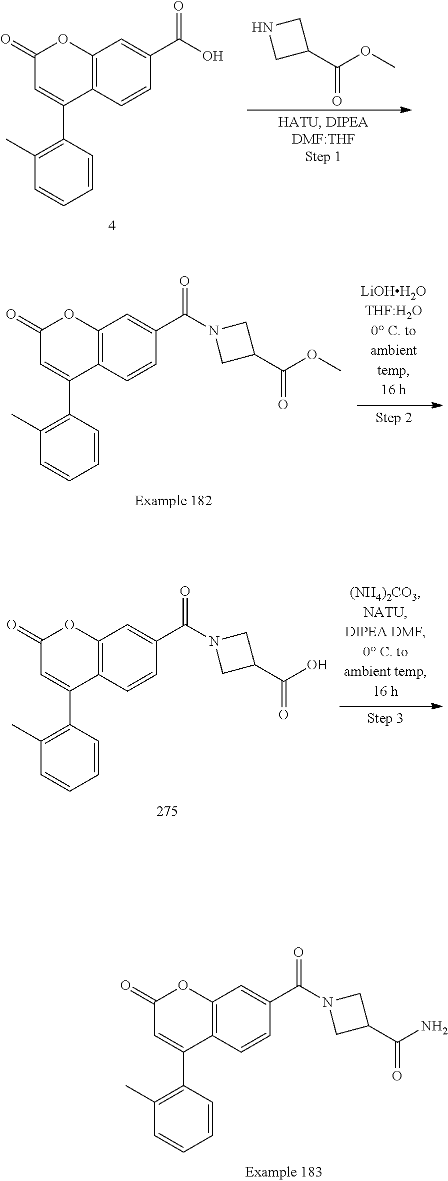

Synthesis of methyl 1-(2-oxo-4-(o-tolyl)-2H-chromene-7-carbonyl) azetidine-3-carboxylate, Example 182 [Step 1]: To a stirred solution of 4-(o-tolyl)-2-oxo-chromene-7-carboxylic acid (4, 50 mg, 0.2 mmol) in DMF (1 mL), HATU (100 mg, 0.3 mmol) and N,N-Diisopropylethylamine (0.1 mL, 0.5 mmol) were added and the reaction mixture was stirred for 30 min. Then methyl azetidine-3-carboxylate (40 mg, 0.4 mmol) and N,N-Diisopropylethylamine (0.1 mL, 0.5 mmol) were added and the reaction mixture was stirred at ambient temperature for 16 h. TLC showed full conversion of starting material with formation of new polar spots. The reaction mixture was diluted with EtOAc and washed with water and brine. The organic phase was dried over anhydrous Na₂SO₄, filtered and evaporated. The compound was purified by reverse phase prep-HPLC and lyophilized to yield methyl 1-(2-oxo-4-(o-tolyl)-2H-chromene-7-carbonyl) azetidine-3-carboxylate (Example 182, 25 mg). LCMS (ESI) Calcd. for C₂₂H₁₉NO₅: 377, found [M+H]⁺=378. ¹H NMR (400 MHz, DMSO-d₆) δ 7.66 (d, 1H), 7.51 (dd, 1H), 7.48-7.41 (m, 2H), 7.37 (t, 1H), 7.28 (d, 1H), 7.05 (d, 1H), 6.50 (s, 1H), 4.51 (br s, 1H), 4.41 (br s, 1H), 4.27 (t, 1H), 4.15-4.11 (m, 1H), 3.67 (s, 3H), 3.63-3.57 (m, 1H), 2.13 (s, 3H).

Synthesis of 1-(2-oxo-4-(o-tolyl)-2H-chromene-7-carbonyl) azetidine-3-carboxylic acid, 275 [Step 2]: To a stirred solution of methyl 1-(2-oxo-4-(o-tolyl)-2H-chromene-7-carbonyl) azetidine-3-carboxylate (Example 182, 130 mg, 0.3 mmol) in THF (5 mL) and water (1 mL) mixture, LiOH·H₂O (20 mg, 0.5 mmol) was added and the reaction mixture was stirred at ambient temperature for 16 h. TLC showed full conversion of starting material with formation of new polar spot. The reaction mixture was evaporated. The mixture was acidified with 1N HCl to pH 3 and extracted with EtOAc. The organic phase was washed with brine, dried over anhydrous Na₂SO₄, filtered and evaporated to yield 1-(2-oxo-4-(o-tolyl)-2H-chromene-7-carbonyl) azetidine-3-carboxylic acid (275, 110 mg). LCMS (ESI) calculated for C₂₁H₁₇NO₅: 363, found [M+H]⁺=364.

Synthesis of 1-(2-oxo-4-(o-tolyl)-2H-chromene-7-carbonyl) azetidine-3-carboxamide, Example 183 [Step 3]: To a stirred solution of 1-(2-oxo-4-(o-tolyl)-2H-chromene-7-carbonyl) azetidine-3-carboxylic acid (275, 130 mg, 0.4 mmol) in DMF (2 mL) were added HOBt (75 mg, 0.5 mmol), EDC·HCl (105 mg, 0.5 mmol) and DIPEA (0.2 mL, 1.4 mmol) followed by (NH₄)₂CO₃ (140 mg, 1.4 mmol) and stirred at ambient temperature for 16 h. TLC showed complete consumption of starting material to form new polar spot. The reaction mixture was diluted with water and extracted with EtOAc. The organic layer was washed with brine, dried over anhydrous Na₂SO₄ and concentrated. The product was purified by reverse phase prep-HPLC and lyophilized to yield 1-(2-oxo-4-(o-tolyl)-2H-chromene-7-carbonyl) azetidine-3-carboxamide (Example 183, 45 mg). LCMS (ESI) Calcd. for C₂₁H₁₈N₂O₄: 362, found [M H]⁺=363.1. ¹H NMR (400 MHz, DMSO-d₆) δ 7.65 (d, 1H), 7.52-7.35 (m, 5H), 7.28 (d, 1H), 7.08-7.04 (m, 2H), 6.49 (s, 1H), 4.43 (m, 1H), 4.32-4.27 (m, 1H), 4.18-4.14 (m, 1H), 4.07-4.03 (m, 1H), 3.38-3.35 (m, 1H), 2.13 (s, 3H).

### Example 184: Synthesis of 7-(((1H-tetrazol-5-yl)methyl)amino)-4-(o-tolyl)-2H-chromen-2-one

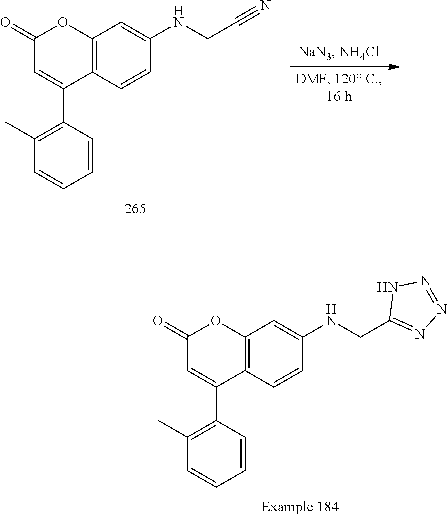 [SMILES: Cc1ccccc1-c1cc(=O)oc2cc(NCC#N)ccc12 ; Cc1ccccc1-c1cc(=O)oc2cc(NCc3nnn[nH]3)ccc12]

Synthesis of 7-(((1H-tetrazol-5-yl)methyl)amino)-4-(o-tolyl)-2H-chromen-2-one, Example 184: A round bottom flask was charged with 2-((2-oxo-4-(o-tolyl)-2H-chromen-7-yl)amino) acetonitrile (265, 100 mg, 0.3 mmol), NH₄Cl (185 mg, 3.4 mmol), and NaN₃ (135 mg, 0.4 mmol) in DMF (4 mL) and heated at 110° C. for 16 h. The reaction mixture was diluted with water and extracted with EtOAc. The organic layer was washed with cold water and brine, dried over anhydrous Na₂SO₄. and concentrated. The product was purified by reverse phase HPLC and lyophilized to afford 7-(((1H-tetrazol-5-yl)methyl)amino)-4-(o-tolyl)-2H-chromen-2-one (Example 184, 57 mg). LCMS (ESI) Calcd. for C₁₈H₁₅N₅O₂: 333, found [M+H]⁺=334. ¹H NMR (400 MHz, DMSO-d₆) δ 7.41-7.29 (m, 4H), 7.19 (d, 2H), 6.65 (d, 1H), 6.57 (d, 2H), 5.88 (s, 1H), 4.61 (d, 2H), 2.09 (s, 3H).

### Example 185: Synthesis of 7-((isoxazol-3-ylmethyl)amino)-4-(o-tolyl)-2H-chromen-2-one

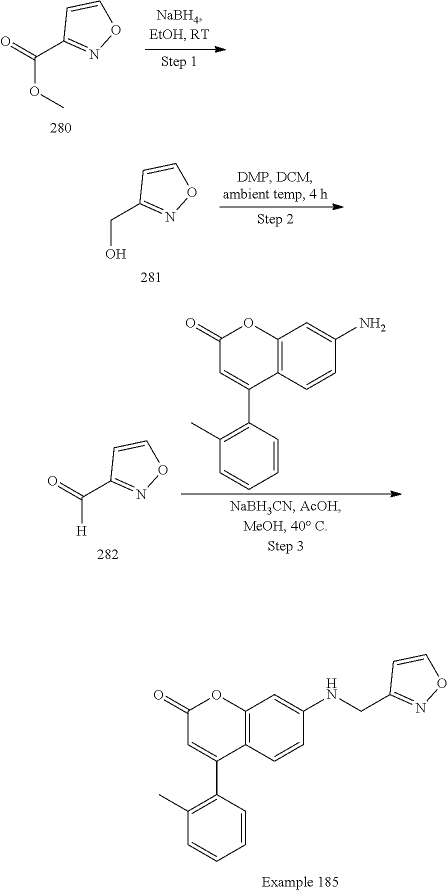

Synthesis of isoxazol-3-ylmethanol, 281 [Step 1]: To a stirred solution of methyl isoxazole-3-carboxylate (280, 500 mg, 3.5 mmol) in ethanol (5 mL) was added NaBH₄ (200 mg, 5.3 mmol) at 0° C. and stirred at ambient temperature for 16 h. After completion, the reaction mixture was evaporated to afford product. The product material was quenched with water and extracted with EtOAc. The organic layer was washed with brine, dried over anhydrous Na₂SO₄, and concentrated to afford isoxazol-3-yl methanol (281, 120 mg). The product material was used for next step without further purification. ¹H NMR (400 MHz, DMSO-d₆) § 8.83 (s, 1H), 6.54 (s, 1H), 5.47-5.45 (m, 1H), 4.53 (d, 2H).

Synthesis of isoxazole-3-carbaldehyde, 282 [Step 2]: To a stirred solution of isoxazol-3-ylmethanol (281, 100 mg, 1.0 mmol) in dichloromethane (5 mL) was added DMP (470 mg, 1.1 mmol) at ambient temperature and stirred for 6 h. The reaction mass was filtered through celite bed and washed with dichloromethane. The filtrate was evaporated to afford isoxazole-3-carbaldehyde (282, 90 mg). The product material was immediately used for next step without further purification. ¹H NMR (400 MHz, DMSO-d₆) δ 10.16 (s, 1H), 9.20 (d, 1H), 7.0 (d, 1H). ¹H NMR contains extra peaks.

Synthesis of 7-((isoxazol-3-ylmethyl)amino)-4-(o-tolyl)-2H-chromen-2-one, Example 185 [Step 3]: To a solution of 7-amino-4-(o-tolyl)-2H-chromen-2-one (247, 120 mg, 0.5 mmol) in methanol (2 mL) was added isoxazole-3-carbaldehyde (70 mg, 0.7 mmol) followed by AcOH (catalytic amount) at ambient temperature and stirred at 40° C. for 16 h. After that sodium cyanoborohydride (120 mg, 1.9 mmol) was added and stirring was continued at ambient temperature for 4 h. The reaction mixture was concentrated under reduced pressure & diluted with EtOAc and washed with water and brine, dried over anhydrous sodium sulfate, and concentrated under reduced pressure. The product was purified by reverse phase prep-HPLC and lyophilized to afford 7-((isoxazol-3-ylmethyl)amino)-4-(o-tolyl)-2H-chromen-2-one (Example 185, 17 mg). LCMS (ESI) Calcd. for C₂₀H₁₆N₂O₃: 332, found [M+H]⁺=333. ¹H NMR (400 MHz, DMSO-d₆) δ 8.85 (d, 1H), 7.40-7.30 (m, 4H), 7.19 (d, 1H), 6.68-6.52 (m, 4H), 5.89 (s, 2H), 4.47 (d, 1H), 2.10 (s, 3H).

### Example 186-187: Synthesis of (S)-1-((S)-3-(4-(2-chloro-4-fluorophenyl)-2-oxo-2H-chromen-7-yl)-2-methylpropanoyl)piperidine-3-carboxylic acid, (S)-1-((R)-3-(4-(2-chloro-4-fluorophenyl)-2-oxo-2H-chromen-7-yl)-2-methylpropanoyl)piperidine-3-carboxylic acid

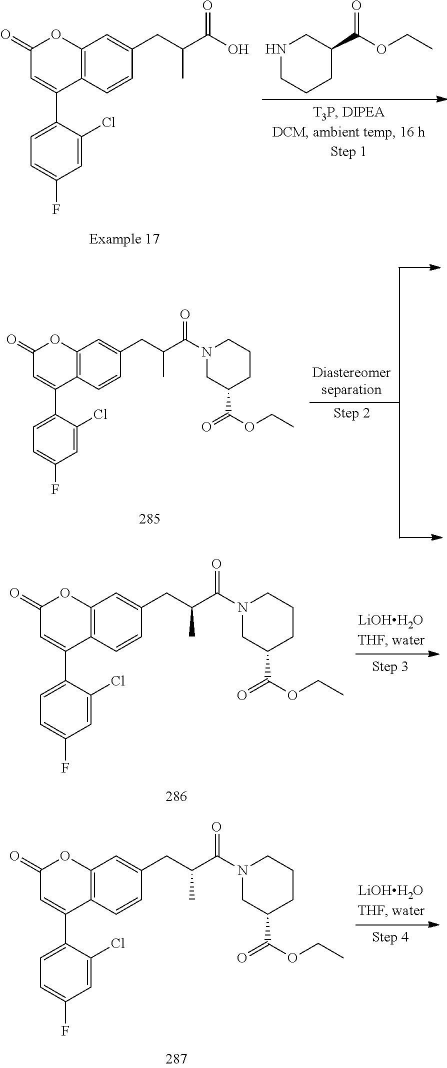 [SMILES: CC(Cc1ccc2c(-c3ccc(F)cc3Cl)cc(=O)oc2c1)C(=O)O ; CCOC(=O)[C@H]1CCCNC1 ; CCOC(=O)[C@H]1CCCN(C(=O)C(C)Cc2ccc3c(-c4ccc(F)cc4Cl)cc(=O)oc3c2)C1 ; CCOC(=O)[C@H]1CCCN(C(=O)[C@@H](C)Cc2ccc3c(-c4ccc(F)cc4Cl)cc(=O)oc3c2)C1 ; CCOC(=O)[C@H]1CCCN(C(=O)[C@H](C)Cc2ccc3c(-c4ccc(F)cc4Cl)cc(=O)oc3c2)C1]

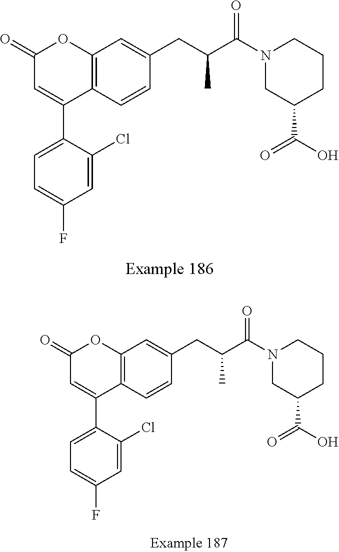 [SMILES: C[C@@H](Cc1ccc2c(-c3ccc(F)cc3Cl)cc(=O)oc2c1)C(=O)N1CCC[C@H](C(=O)O)C1 ; C[C@H](Cc1ccc2c(-c3ccc(F)cc3Cl)cc(=O)oc2c1)C(=O)N1CCC[C@H](C(=O)O)C1]

Synthesis of ethyl (3S)-1-(3-(4-(2-chloro-4-fluorophenyl)-2-oxo-2H-chromen-7-yl)-2-methylpropanoyl)piperidine-3-carboxylate, 285 [Step 1]: To a stirred solution of 3-(4-(2-chloro-4-fluorophenyl)-2-oxo-2H-chromen-7-yl)-2-methylpropanoic acid (Example 17, 600 mg, 1.7 mmol) and ethyl (3S)-piperidine-3-carboxylate (314 mg, 2 mmol) in dichloromethane (10 mL) was added DIPEA (0.9 mL, 9 mmol) followed by 50% T₃P in ethyl acetate (0.7 mL, 2.9 mmol) at 0° C. and stirred at ambient temperature for 2 h. The reaction mixture was diluted with dichloromethane and washed with water, organic layer was collected, washed with brine, dried over anhydrous Na₂SO₄, and concentrated under reduced pressure to afford product which was purified by column chromatography to afford ethyl (3S)-1-(3-(4-(2-chloro-4-fluorophenyl)-2-oxo-2H-chromen-7-yl)-2-methylpropanoyl)piperidine-3-carboxylate (285, 450 mg). LCMS (ESI) Calcd. for C₂₇H₂₇ClFO₅: 499, found [M+H]⁺: 500.

Synthesis of ethyl (S)-1-((R)-3-(4-(2-chloro-4-fluorophenyl)-2-oxo-2H-chromen-7-yl)-2-methylpropanoyl)piperidine-3-carboxylate, 286, and ethyl (S)-1-((S)-3-(4-(2-chloro-4-fluorophenyl)-2-oxo-2H-chromen-7-yl)-2-methylpropanoyl)piperidine-3-carboxylate, 287 [Step 2]: Ethyl (3S)-1-(3-(4-(2-chloro-4-fluorophenyl)-2-oxo-2H-chromen-7-yl)-2-methylpropanoyl)piperidine-3-carboxylate (285, 450 mg, 0.90 mmol) was separated via NP chiral separation to get Peak 1 as ethyl (S)-1-((S)-3-(4-(2-chloro-4-fluorophenyl)-2-oxo-2H-chromen-7-yl)-2-methylpropanoyl)piperidine-3-carboxylate (286, 150 mg) and Peak 2 as ethyl (S)-1-((R)-3-(4-(2-chloro-4-fluorophenyl)-2-oxo-2H-chromen-7-yl)-2-methylpropanoyl)piperidine-3-carboxylate (287, 150 mg). The absolute stereochemistry of these Examples was not determined.

Peak 1: Ethyl(S)-1-((S)-3-(4-(2-chloro-4-fluorophenyl)-2-oxo-2H-chromen-7-yl)-2-methylpropanoyl)piperidine-3-carboxylate. LCMS (ESI) Calcd. for C₂₇H₂₇ClFO₅: 499, found [M+H]⁺: 500. NMR (400 MHz, DMSO-d₆ at 100° C.) δ 7.72 (d, 1H), 7.57 (d, 1H), 7.45-7.41 (m, 1H), 7.34 (s, 1H), 7.12 (t, 1H), 6.93 (d, 1H), 6.46 (d, 1H), 4.02 (d, 2H), 3.77 (m, 1H), 3.42 (s, 1H), 3.32 (d, 1H), 3.12 (d, 1H), 3.12 (s, 1H), 3.02 (s, 1H), 2.94 (s, 1H), 1.83 (s, 1H), 1.76 (s, 1H), 1.65 (d, 1H), 1.54 (t, 1H), 1.41 (d, 1H), 1.23 (s, 3H), 1.16-1.11 (m, 3H).

Peak 2: Ethyl(S)-1-((R)-3-(4-(2-chloro-4-fluorophenyl)-2-oxo-2H-chromen-7-yl)-2-methylpropanoyl)piperidine-3-carboxylate. LCMS (ESI) Calcd. for C₂₇H₂₇ClFO₅: 499, found [M+H]⁺: 500. NMR (400 MHz, DMSO-d₆) δ 7.71 (d, 1H), 7.55 (d, 1H), 7.46 (d, 1H), 7.39 (s, 1H), 7.15 (t, 1H), 6.94 (d, 1H), 6.44 (d, 1H), 4.04 (d, 2H), 3.80-3.71 (m, 2H), 3.03 (t, 2H), 2.69 (d, 2H), 2.01 (t, 1H), 1.85 (t, 1H), 1.76 (s, 1H), 1.50 (d, 2H), 1.18-1.13 (m, 3H), 1.02 (s, 4H).

Chiral separation was performed on an Agilent 1200 series instrument. Column was a CHIRALPAK IG (250×21 mm), 5μ. Operating at ambient temperature and flow rate is 21.0 ml/min. Mobile phase was 0.1% Isopropylamine in the mixture of 70% Hexane, 15% Dichloromethane and 15% Ethanol. Held this isocratic mixture for up to 20 min. with detection at 284 nm wavelength.

Synthesis of (S)-1-((S)-3-(4-(2-chloro-4-fluorophenyl)-2-oxo-2H-chromen-7-yl)-2-methylpropanoyl)piperidine-3-carboxylic acid, Example 186 [Step 3]: To a stirred solution of ethyl (S)-1-((R)-3-(4-(2-chloro-4-fluorophenyl)-2-oxo-2H-chromen-7-yl)-2-methylpropanoyl)piperidine-3-carboxylate (286, 150 mg, 0.3 mmol) in THF (2.5 mL) was added the solution of LiOH·H₂O (38 mg, 0.9 mmol) in water (0.5 mL) at ambient temperature. The mixture was stirred for 6 h. at ambient temperature. The reaction mixture was cooled to 0° C. and acidified with 1N HCl solution. It was filtered on sintered funnel and washed with water. The product was purified by reverse phase prep-HPLC purification and lyophilized to afford(S)-1-((S)-3-(4-(2-chloro-4-fluorophenyl)-2-oxo-2H-chromen-7-yl)-2-methylpropanoyl)piperidine-3-carboxylic acid (Example 186, 116 mg). LCMS (ESI) Calcd. for C₂₅H₂₃ClFNO₅: 471, found [M+H]⁺=472. NMR (400 MHz, DMSO-d₆) (at 100° C.) δ 7.60-7.51 (m, 2H), 7.40 (t, 1H), 7.30 (s, 1H), 7.10 (s, 1H), 6.95 (d, 1H), 6.35 (s, 1H), 3.89 (d, 1H), 3.21 (t, 1H), 3.00 (t, 2H), 2.86 (d, 2H), 2.75-2.66 (m, 1H), 1.85 (s, 1H), 1.60 (t, 2H), 1.27 (s, 1H), 1.07 (d, 3H).

Synthesis of (S)-1-((R)-3-(4-(2-chloro-4-fluorophenyl)-2-oxo-2H-chromen-7-yl)-2-methylpropanoyl)piperidine-3-carboxylic acid, Example 187 [Step 4]: To a stirred solution of ethyl (S)-1-((S)-3-(4-(2-chloro-4-fluorophenyl)-2-oxo-2H-chromen-7-yl)-2-methylpropanoyl)piperidine-3-carboxylate (287, 150 mg, 0.3 mmol) in THF (2.5 mL) was added the solution of LiOH· H₂O (38 mg, 0.9 mmol) in water (0.5 mL) at ambient temperature. It was stirred for 6 h. at ambient temperature. The reaction mixture was cooled to 0° C. and acidified with 1N HCl solution. It was filtered on sintered funnel and washed with water. The product was dried under reduced pressure, purified by reverse phase prep-HPLC and lyophilized to afford(S)-1-((R)-3-(4-(2-chloro-4-fluorophenyl)-2-oxo-2H-chromen-7-yl)-2-methylpropanoyl)piperidine-3-carboxylic acid (Example 187, 80 mg). LCMS (ESI) Calcd. for C₂₅H₂₃ClFNO₅: 471, found [M+H]⁺=472. NMR (400 MHz, DMSO-d₆) (at 100° C.) δ 7.61-7.52 (m, 2H), 7.41 (t, 1H), 7.33 (d, 1H), 7.13 (s, 1H), 6.95 (d, 1H), 6.36 (s, 1H), 4.20 (s, 2H), 3.81 (d, 1H), 3.22 (t, 2H), 3.02-2.97 (m, 2H), 2.74-2.67 (m, 2H), 1.85 (s, 1H), 1.57 (t, 2H), 1.06 (d, 3H).

### Example 188: Synthesis of 7-(methyl(oxazol-2-ylmethyl)amino)-4-(o-tolyl)-2H-chromen-2-one

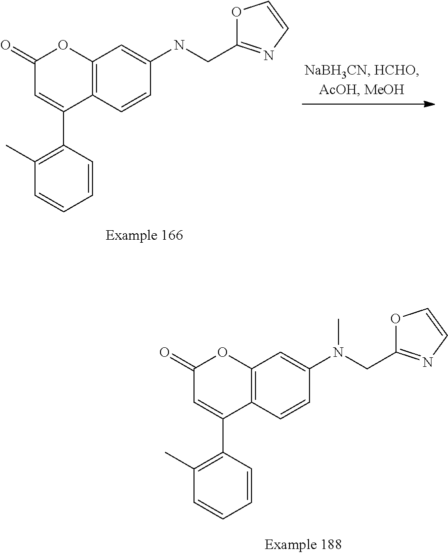 [SMILES: Cc1ccccc1-c1cc(=O)oc2cc(NCc3ncco3)ccc12 ; Cc1ccccc1-c1cc(=O)oc2cc(N(C)Cc3ncco3)ccc12]

Synthesis of 7-(methyl(oxazol-2-ylmethyl)amino)-4-(o-tolyl)-2H-chromen-2-one, Example 188: To a solution of 7-((oxazol-2-ylmethyl)amino)-4-(o-tolyl)-2H-chromen-2-one (Example 166, 100 mg, 0.3 mmol) in methanol (2 mL) was added formaldehyde (0.2 ml, 0.5 mmol) followed by AcOH (catalytic amount) at ambient temperature and stirred at 40° C. for 16 h. After that sodium cyanoborohydride (30 mg, 0.5 mmol) was added and continued stirring at ambient temperature for 4 h. The reaction mixture was concentrated under reduced pressure and diluted with EtOAc and washed with water and brine, dried over anhydrous sodium sulfate and concentrated under reduced pressure. The product was purified by reverse phase prep-HPLC purification and lyophilized to afford 7-(methyl(oxazol-2-ylmethyl)amino)-4-(o-tolyl)-2H-chromen-2-one (Example 188, 13 mg). LCMS (ESI) Calcd. for C₂₁H₁₈N₂O₃: 346, found [M+H]⁺=347. ¹H NMR (400 MHz, DMSO-d₆ at 100° C.) δ 7.97 (s, 1H), 7.38-7.33 (m, 3H), 7.21 (d, 1H), 7.13 (s, 1H), 6.77 (s, 3H), 5.91 (s, 1H), 4.77 (s, 2H), 3.14 (s, 3H), 2.14 (s, 3H).

### Example 189-190: Synthesis of chiral 2-((2-oxo-4-(o-tolyl)-2H-chromen-7-yl)amino)propenamide

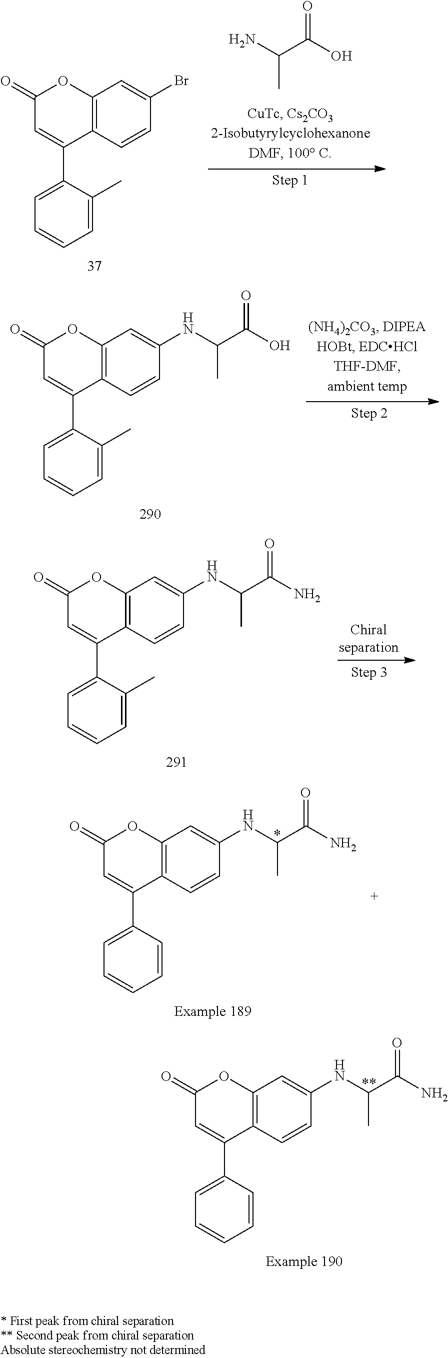

Synthesis of (2-oxo-4-(o-tolyl)-2H-chromen-7-yl) alanine, 290 [Step 1]: An oven dried sealed tube was charged with 7-bromo-4-(o-tolyl)-2H-chromen-2-one (37, 500 mg, 1.6 mmol) and alanine (170 mg, 1.9 mmol) followed by cesium carbonate (1.3 g, 3.1 mmol), copper thiophene 2-carboxylate (60 mg, 0.3 mmol), 2-isobutyrylcyclohexanone (0.2 mL, 1.3 mmol) and DMF (5 mL). The reaction mixture was heated at 100° C. for 12 h. The reaction mixture was partitioned between EtOAc and water. The water layer was collected and acidified up to pH 5-6 using 2N HCl. The aqueous phase was extracted using 15% MeOH-dichloromethane. The organic layer washed with ice cold brine, dried over anhydrous Na₂SO₄, and concentrated under reduced pressure. The product was purified using column chromatography to afford (2-oxo-4-(o-tolyl)-2H-chromen-7-yl) alanine (290, 350 mg). LCMS (ESI) Calcd. for C₁₉H₁₇NO₄: 323, found [M+H]⁺=324.

Synthesis of 2-((2-oxo-4-(o-tolyl)-2H-chromen-7-yl)amino)propenamide, 291 [Step 2]: An oven dried round bottle flask was charged with (2-oxo-4-(o-tolyl)-2H-chromen-7-yl) alanine (290, 350 mg, 1.1 mmol) followed by DMF (2 mL) and THF (2 mL). HOBt (220 mg, 1.6 mmol), EDC·HCl (310 mg, 1.6 mmol) and DIPEA (0.6 mL, 4.3 mmol) were added to the reaction mixture followed by ammonium bicarbonate (415 mg, 4.3 mmol). The reaction mixture was stirred at ambient temperature for 17 h. The reaction mixture was partitioned between 15% MeOH-dichloromethane and water. The organic layer was collected, washed with brine, dried over anhydrous Na₂SO₄, and concentrated under reduced pressure. The product was purified using reverse phase prep-HPLC and lyophilized to afford 2-((2-oxo-4-(o-tolyl)-2H-chromen-7-yl)amino)propanamide (291, 120 mg). LCMS (ESI) Calcd. for C₁₉H₁₈N₂O₃: 322, found [M+H]⁺=323.

Synthesis of chiral 2-((2-oxo-4-(o-tolyl)-2H-chromen-7-yl)amino)propenamide, Example 189 and Example 190 [Step 3]: Two stereoisomers were separated from racemic 2-((2-oxo-4-(o-tolyl)-2H-chromen-7-yl)amino)propenamide (291, 115 mg, 0.4 mmol) using chiral PREP NP to afford Peak 1 as 2-((2-oxo-4-(o-tolyl)-2H-chromen-7-yl)amino)propanamide (Example 189, 46 mg) and Peak 2 as 2-((2-oxo-4-(o-tolyl)-2H-chromen-7-yl)amino)propanamide (Example 190, 40 mg). The absolute stereochemistry of these Examples was not determined.

Example 189: 2-((2-oxo-4-(o-tolyl)-2H-chromen-7-yl)amino)propanamide: LCMS (ESI) Calcd. for C₁₉H₁₈N₂O₃: 322, found [M+H]⁺=323. ¹H NMR (400 MHz, DMSO-d₆): δ 7.51-7.50 (m, 1H), 7.42-7.30 (m, 3H), 7.20-7.18 (m, 1H), 7.09 (s, 1H) 6.90-6.88 (m, 1H), 6.66-6.63 (m, 1H), 6.55-6.52 (m, 1H), 6.44 (s, 1H), 5.87 (s, 1H), 3.94-3.87 (m, 1H), 2.11 (s, 3H), 1.32 (d, 3H).

Example 190: 2-((2-oxo-4-(o-tolyl)-2H-chromen-7-yl)amino)propanamide: LCMS (ESI) Calcd. for LCMS (ESI) Calcd. for C₁₉H₁₈N₂O₃: 322, found [M+H]⁺=323. ¹H NMR (400 MHz, DMSO-d₆): δ 7.51-7.50 (m, 1H), 7.42-7.30 (m, 3H), 7.20-7.18 (m, 1H), 7.08 (s, 1H) 6.90-6.87 (m, 1H), 6.66-6.64 (m, 1H), 6.55-6.53 (m, 1H), 6.45 (s, 1H) 5.87 (s, 1H), 3.93-3.89 (m, 1H) 2.11 (s, 3H), 1.32 (d, 3H).

Chiral PREP METHOD: Chiral separation was performed on an Agilent 1200 series instrument. Column was a CHIRALPAK IG (250×21 mm) 5μ. Operating at ambient temperature and flow rate is 21.0 ml/min. Mobile phase was the mixture of 50% Hexane, 25% Dichloromethane and 25% Ethanol held this isocratic mixture for up to 20 min. with detection at 360 nm wavelength.

### Example 191: Synthesis of 2-(methyl(2-oxo-4-(o-tolyl)-2H-chromen-7-yl)amino)-N-(methylsulfonyl)acetamide

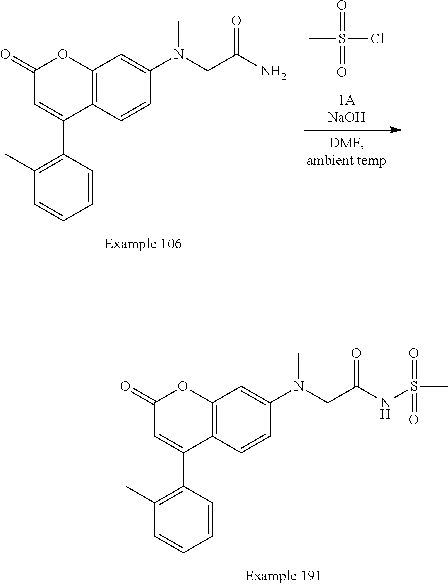

Synthesis of 2-(methyl(2-oxo-4-(o-tolyl)-2H-chromen-7-yl)amino)-N-(methylsulfonyl)acetamide, Example 191 [Step 1]: To a solution of 2-(methyl(2-oxo-4-(o-tolyl)-2H-chromen-7-yl)amino)acetamide (Example 106, 100 mg, 0.3 mmol) in DMF (3 mL) was added NaH (60% mineral oil) (19 mg, 0.5 mmol) at 0° C. and stirred at ambient temperature for 15 min. After that methanesulfonyl chloride (0.026 mL, 0.4 mmol) was added at 0° C. and stirred for 5 hrs at ambient temperature. The product was purified by reverse phase prep-HPLC and lyophilized to afford 2-(methyl(2-oxo-4-(o-tolyl)-2H-chromen-7-yl)amino)-N-(methylsulfonyl)acetamide (Example 191, 17 mg). LCMS (ESI) Calcd. for C₂₀H₂₀N₂O₅S: 400, found, [M−H]⁻=399. ¹H NMR (400 MHz, DMSO-d₆): δ 7.22-7.17 (m, 2H), 7.15-7.10 (m, 3H), 6.86-6.84 (m, 1H), 6.79-6.78 (d, 1H), 5.97 (s, 1H), 4.58 (s, 2H), 3.16 (s, 3H), 2.97 (s, 3H), 2.15 (s, 3H).

### Example 192: Synthesis of 7-(((1-methyl-1H-pyrazol-5-yl)methyl)amino)-4-(o-tolyl)-2H-chromen-2-one

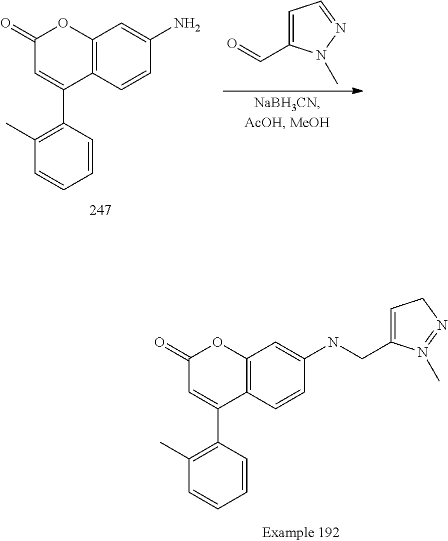

Synthesis of 7-(((1-methyl-1H-pyrazol-5-yl)methyl)amino)-4-(o-tolyl)-2H-chromen-2-one, Example 192: To a solution of 7-amino-4-(o-tolyl)-2H-chromen-2-one (247, 200 mg, 0.8 mmol) in methanol (2 mL) was added 1-methyl-1H-pyrazole-5-carbaldehyde (130 mg, 1.2 mmol) followed by AcOH (catalytic amount) at ambient temperature and stirred at 60° C. for 16 h. After that sodium cyanoborohydride (200 mg, 3.2 mmol) was added and stirring was continued at ambient temperature for 4 h. The reaction mixture was concentrated under reduced pressure. The product was diluted with EtOAc and washed with water and brine, dried over anhydrous sodium sulfate and concentrated under reduced pressure. The product was purified by reverse phase prep-HPLC and lyophilized to afford 7-(((1-methyl-1H-pyrazol-5-yl)methyl)amino)-4-(o-tolyl)-2H-chromen-2-one (Example 192, 43 mg). LCMS (ESI) Calcd. for C₂₁H₁₉N₃O₂: 345; [M−H]⁻=344. ¹H NMR (400 MHz, DMSO-d₆): δ 7.40-7.30 (m, 4H), 7.23-7.19 (m, 2H), 6.67-6.60 (m, 3H), 6.22 (s, 1H), 5.87 (s, 1H), 4.42 (d, 2H), 3.80 (s, 3H), 2.11 (s, 3H).

### Example 193: Synthesis of 7-(methyl((1-methyl-1H-pyrazol-5-yl)methyl)amino)-4-(o-tolyl)-2H-chromen-2-one

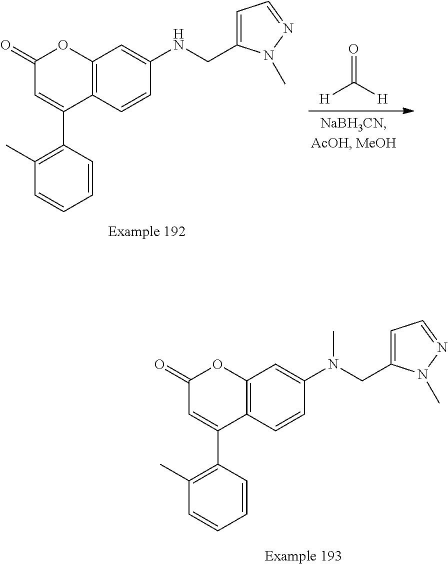

Synthesis of 7-(methyl((1-methyl-1H-pyrazol-5-yl)methyl)amino)-4-(o-tolyl)-2H-chromen-2-one, Example 193: To a solution of 7-(((1-methyl-1H-pyrazol-5-yl)methyl)amino)-4-(o-tolyl)-2H-chromen-2-one (Example 192, 100 mg, 0.3 mmol) in methanol (2 mL) was added formaldehyde (0.1 ml, 0.4 mmol) followed by AcOH (catalytic amount) at ambient temperature and stirred at 60° C. for 16 h. After that sodium cyanoborohydride (25 mg, 0.4 mmol) was added and stirring was continued at ambient temperature for 4 h. The reaction mixture was concentrated under reduced pressure to yield a residue. The product was diluted with EtOAc and washed with water and brine, dried over anhydrous sodium sulfate, and concentrated under reduced pressure. The product was purified by reverse phase prep-HPLC and lyophilized to afford 7-(methyl((1-methyl-1H-pyrazol-5-yl)methyl)amino)-4-(o-tolyl)-2H-chromen-2-one (Example 193, 18 mg). LCMS (ESI) Calcd. for C₂₂H₂₁N₃O₂: 359; [M+H]⁺=360. ¹H NMR (400 MHz, DMSO-d₆): δ 7.41-7.20 (m, 5H), 6.76 (s, 1H), 6.73 (s, 2H), 5.93 (s, 1H), 5.92 (s, 1H), 4.74 (s, 2H), 3.77 (s, 3H), 3.06 (s, 3H), 2.11 (s, 3H).

### Example 194-195: Synthesis of chiral 2-(methyl(2-oxo-4-phenyl-2H-chromen-7-yl)amino)propenamide

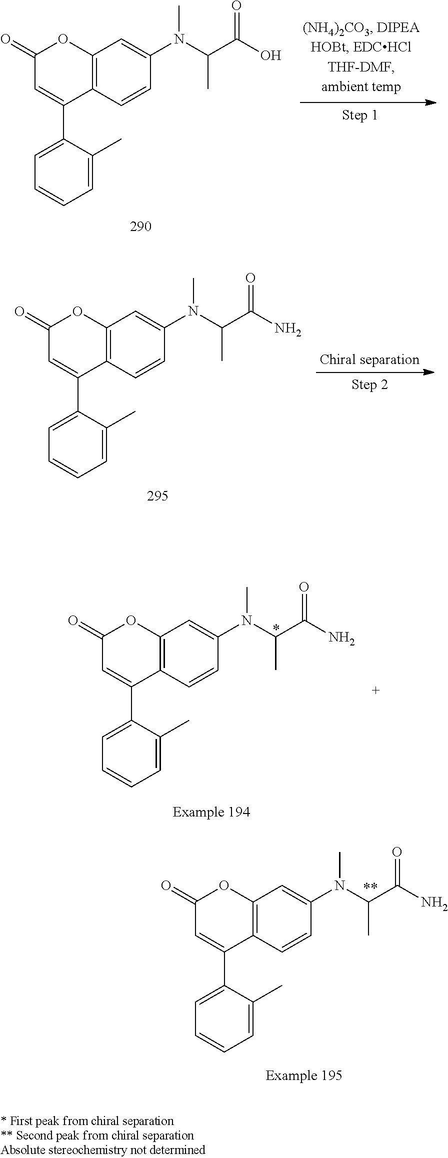 [SMILES: Cc1ccccc1-c1cc(=O)oc2cc(N(C)C(C)C(=O)O)ccc12 ; Cc1ccccc1-c1cc(=O)oc2cc(N(C)C(C)C(N)=O)ccc12 ; Cc1ccccc1-c1cc(=O)oc2cc(N(C)C(C)C(N)=O)ccc12 ; Cc1ccccc1-c1cc(=O)oc2cc(N(C)C(C)C(N)=O)ccc12]

Synthesis of 2-(methyl(2-oxo-4-(o-tolyl)-2H-chromen-7-yl)amino)propenamide, 295 [Step 1]: An oven dried round bottle flask charged with N-methyl-N-(2-oxo-4-(o-tolyl)-2H-chromen-7-yl) alanine (290, 280 mg, 0.8 mmol) followed by DMF (2 mL) and THF (2 mL). HOBt (170 mg, 1.2 mmol), EDC·HCl (240 mg, 1.2 mmol), DIPEA (0.5 mL, 3.3 mmol) and ammonium bicarbonate (320 mg, 3.3 mmol) were added to the reaction mixture. The reaction mixture was stirred at ambient temperature for 17 h. The reaction mixture was partitioned between 15% MeOH-dichloromethane and water. Organic extract was collected, washed with brine, dried over anhydrous Na₂SO₄, and concentrated under reduced pressure. The product was purified using reverse phase prep-HPLC to afford 2-(methyl(2-oxo-4-(o-tolyl)-2H-chromen-7-yl)amino)propanamide (295, 110 mg). LCMS (ESI) Calcd. for C₂₀H₂₀N₂O₃: 336, found [M+H]⁺=337.

Synthesis of chiral 2-(methyl(2-oxo-4-phenyl-2H-chromen-7-yl)amino)propenamide, Examples 194 and 195 [Step 2]: Two isomers were separated from racemic 2-(methyl(2-oxo-4-(o-tolyl)-2H-chromen-7-yl)amino)propanamide (295, 105 mg, 0.3 mmol) using chiral PREP NP purification to afford Peak 1 as 2-(methyl(2-oxo-4-phenyl-2H-chromen-7-yl)amino)propanamide (Example 194, 39 mg) and Peak 2 as 2-(methyl(2-oxo-4-(o-tolyl)-2H-chromen-7-yl)amino)propanamide (Example 195, 41 mg). The absolute stereochemistry of these Examples was not determined. 0525 Example 194: 2-(methyl(2-oxo-4-phenyl-2H-chromen-7-yl)amino)propanamide: LCMS (ESI) Calcd. for C₂₀H₂₀N₂O₃: 336, found: [M+H]⁺=337. ¹H NMR (400 MHz, DMSO-d₆): 7.42-7.37 (m, 3H), 7.36-7.31 (m, 1H), 7.22-7.20 (m, 1H), 7.13 (s, 1H), 6.73 (s, 3H), 5.93 (s, 1H), 4.53-4.49 (m, 1H), 2.90 (s, 3H) 2.33 (s, 3H), 1.33 (d, 3H).

Example 195: 2-(methyl(2-oxo-4-(o-tolyl)-2H-chromen-7-yl)amino)propanamide: LCMS (ESI) Calcd. for C₂₀H₂₀N₂O₃: 336, found: [M+H]⁺=337. ¹H NMR (400 MHz, DMSO-d₆): δ 7.42-7.37 (m, 3H), 7.35-7.31 (m, 1H), 7.22-7.19 (m, 1H), 7.13 (s, 1H), 6.73 (s, 3H), 5.93 (s, 1H), 4.51-4.49 (m, 1H), 2.90 (s, 3H) 2.12 (s, 3H), 1.33 (d, 3H).

NP PREP METHOD: Chiral separation was performed on an Agilent 1200 series instrument. Column was a CHIRALPAK IG (250×21 mm) 5μ. Operating at ambient temperature and flow rate is 21.0 mL/min. Mobile phase was the mixture of 65% hexane, 17.5% dichloromethane and 17.5% ethanol, held isocratic for up to 22 min. with detection at 365 nm wavelength.

Example 196, Example 174: Synthesis of chiral isomers of N-((1-(2-oxo-4-(o-tolyl)-2H-chromene-7-carbonyl)pyrrolidin-3-yl)methyl)acetamide

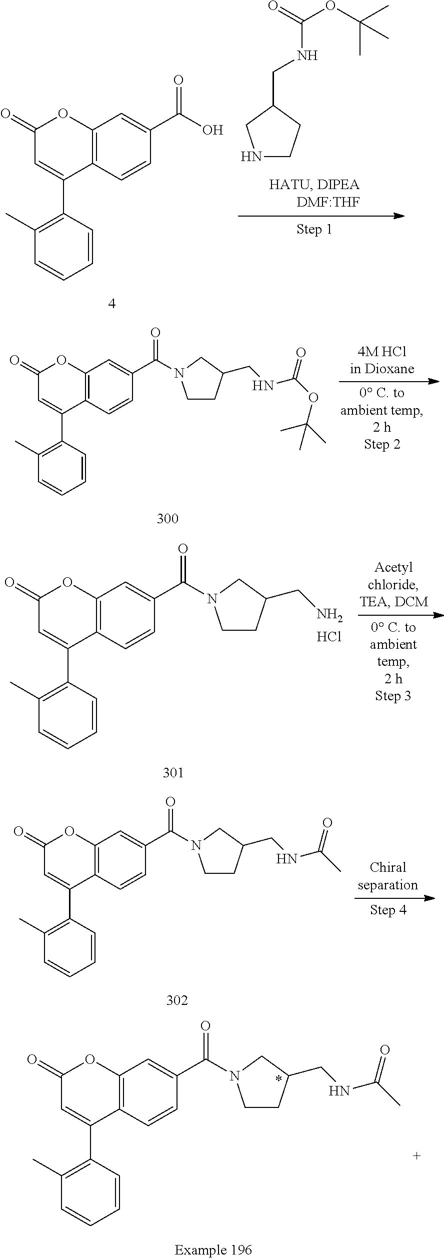

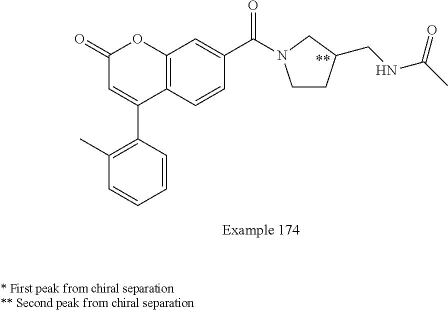 [SMILES: CC(=O)NCC1CCN(C(=O)c2ccc3c(-c4ccccc4C)cc(=O)oc3c2)C1]

Synthesis of tert-butyl ((1-(2-oxo-4-(o-tolyl)-2H-chromene-7-carbonyl)pyrrolidin-3-yl)methyl)carbamate, 300 [Step 1]: To a solution of 2-oxo-4-(o-tolyl)-2H-chromene-7-carboxylic acid (4, 300 mg, 1.1 mmol) in DMF (1.5 mL), HATU (610 mg, 1.6 mmol) was added followed by addition of N,N-Diisopropylethylamine (0.75 mL, 4.3 mmol). The reaction mixture was stirred for 15 min. at ambient temperature under nitrogen balloon. Then tert-butyl (pyrrolidine-3-ylmethyl)carbamate (430 mg, 2.1 mmol) was added to the reaction mixture and stirred at ambient temperature for 16 h. The reaction mixture was diluted with EtOAc and washed with 5% aqueous K₂CO₃ solution. The organic phase was then washed with ice cold water and brine, then was dried over anhydrous Na₂SO₄, filtered, and evaporated. The product was purified through reverse phase prep-HPLC and lyophilized to afford tert-butyl ((1-(2-oxo-4-(o-tolyl)-2H-chromene-7-carbonyl)pyrrolidin-3-yl)methyl)carbamate (300, 300 mg). LCMS (ESI) Calcd. For C₂₇H₃₀N₂O₅: 462; [M+H]⁺=463.

Synthesis of 7-(3-(aminomethyl)pyrrolidine-1-carbonyl)-4-(o-tolyl)-2H-chromen-2-one hydrochloride, 301 [Step 2]: To a solution at ambient temperature butyl ((1-(2-oxo-4-(o-tolyl)-2H-chromene-7-carbonyl)pyrrolidin-3-yl)methyl)carbamate (300, 300 mg, 0.6 mmol) in dichloromethane (1 ml), 4M HCl in dioxane (1.0 mL, 3.8 mmol) was added at 0° C. and stirred at ambient temperature for 2 h. The reaction mixture was concentrated under reduced pressure and lyophilized to afford 7-(3-(aminomethyl)pyrrolidine-1-carbonyl)-4-(o-tolyl)-2H-chromen-2-one hydrochloride (301, 220 mg). LCMS (ESI) Calcd. For C₂₂H₂₂N₂O₃: 362; [M+H]⁺=363.

Synthesis of N-((1-(2-oxo-4-(o-tolyl)-2H-chromene-7-carbonyl)pyrrolidin-3-yl)methyl)acetamide, 302 [Step 3]: To a stirred suspension of 7-(3-(aminomethyl)pyrrolidine-1-carbonyl)-4-(o-tolyl)-2H-chromen-2-one hydrochloride (301, 200 mg, 0.6 mmol) in dichloromethane (1 mL), triethylamine (0.4 mL, 2.8 mmol) was added followed by addition of acetyl chloride (0.1 mL, 1.7 mmol) at 0° C. and stirring was continued at ambient temperature for 2 h. The reaction was monitored by LCMS. The reaction mixture was concentrated under reduced pressure and partitioned with ethyl acetate and water. The organic layer was separated, washed with water, dried over anhydrous Na₂SO₄, filtered and concentrated to afford the product which was purified through reverse phase prep-HPLC to afford N-((1-(2-oxo-4-(o-tolyl)-2H-chromene-7-carbonyl)pyrrolidin-3-yl)methyl)acetamide (302, 150 mg). LCMS (ESI) Calcd. for C₂₄H₂₄N₂O₄: 404; [M+H]⁺=405. ¹H NMR (400 MHz, DMSO-d₆ at 100° C.) δ 7.61 (br s, 1H), 7.54 (s, 1H), 7.47-7.35 (m, 4H), 7.27 (d, 1H), 7.05 (d, 1H), 6.39 (s, 1H), 3.53-3.46 (m, 3H), 3.22-3.11 (m, 3H), 2.38-2.33 (m, 1H), 2.16 (s, 3H), 2.00-1.95 (m, 1H), 1.81 (s, 3H), 1.67-1.62 (m, 1H).

Synthesis of chiral isomers of N-((1-(2-oxo-4-(o-tolyl)-2H-chromene-7-carbonyl)pyrrolidin-3-yl)methyl)acetamide, Example 196 and Example 174 [Step 4]: N-((1-(2-oxo-4-(o-tolyl)-2H-chromene-7-carbonyl)pyrrolidin-3-yl)methyl)acetamide (302, 140 mg) was submitted for chiral HPLC NP separation and lyophilized to afford Peak 1 as N-((1-(2-oxo-4-(o-tolyl)-2H-chromene-7-carbonyl)pyrrolidin-3-yl)methyl)acetamide (Example 196, 27 mg) and Peak 2 as N-((1-(2-oxo-4-(o-tolyl)-2H-chromene-7-carbonyl)pyrrolidin-3-yl)methyl)acetamide (Example 174, 20 mg).

Example 196: (S)—N-((1-(2-oxo-4-(o-tolyl)-2H-chromene-7-carbonyl)pyrrolidin-3-yl)methyl)acetamide (Peak 1): LCMS (ESI) Calcd. for C₂₄H₂₄N₂O₄: 404; [M+H]⁺=405. ¹H NMR (400 MHz, DMSO-d₆ at 100° C.) δ 7.61 (br s, 1H), 7.54 (s, 1H), 7.47-7.35 (m, 4H), 7.27 (d, 1H), 7.05 (d, 1H), 6.39 (s, 1H), 3.53-3.46 (m, 3H), 3.22-3.11 (m, 3H), 2.38-2.33 (m, 1H), 2.16 (s, 3H), 2.00-1.95 (m, 1H), 1.81 (s, 3H), 1.67-1.62 (m, 1H).

Example 174: N-((1-(2-oxo-4-(o-tolyl)-2H-chromene-7-carbonyl)pyrrolidin-3-yl)methyl)acetamide (Peak 2): LCMS (ESI) Calcd. for C₂₄H₂₄N₂O₄: 404; [M+H]⁺=405. ¹H NMR (400 MHz, DMSO-d₆ at 100° C.) δ 7.61 (br s, 1H), 7.54 (s, 1H), 7.47-7.35 (m, 4H), 7.27 (d, 1H), 7.05 (d, 1H), 6.39 (s, 1H), 3.53-3.46 (m, 3H), 3.22-3.11 (m, 3H), 2.38-2.33 (m, 1H), 2.16 (s, 3H), 2.00-1.95 (m, 1H), 1.81 (s, 3H), 1.67-1.62 (m, 1H).

Prep-SFC method: Chiral separation was performed on an Agilent 1200 series instrument. Column was a C AMYLOSE A (250×21 mm) 5μ, operating at ambient temperature and flow rate was 21.0 ml/min. Mobile phase was a mixture of 60% hexane and 40% ethanol, held isocratic for up to 30 min. with detection at 280 nm wavelength.

A reaction with known conformer (tert-butyl (R)-(pyrrolidin-3-ylmethyl)carbamate) was attempted and after comparing with chiral HPLC of both isomers, it was confirmed that Peak 1 is the S-isomer and Peak 2 is the R-isomer.

### Example 198-199: Synthesis of chiral 3-methoxy-2-((2-oxo-4-phenyl-2H-chromen-7-yl)amino)propenamide

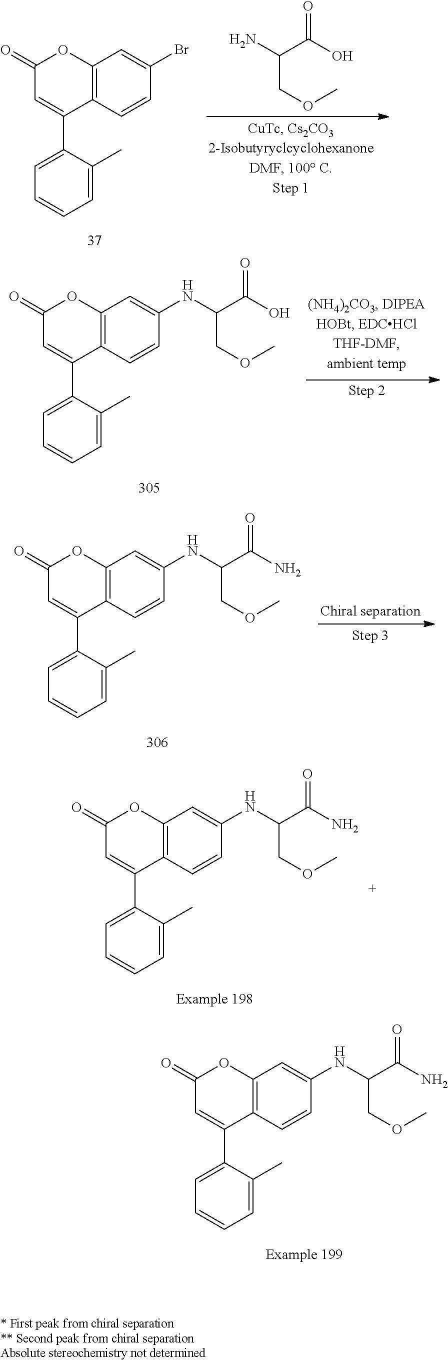

Synthesis of O-methyl-N-(2-oxo-4-(o-tolyl)-2H-chromen-7-yl) serine, 305 [Step 1]: An oven dried sealed tube was charged with 7-bromo-4-(o-tolyl)-2H-chromen-2-one (37, 1 g, 3.2 mmol) and DMF (10 mL) followed by O-methylserine (455 mg, 3.8 mmol), cesium carbonate (2.1 g, 6.3 mmol), copper thiophene 2-carboxylate (120 mg, 0.6 mmol) and 2-isobutyrylcyclohexanone (0.4 mL, 2.5 mmol). The reaction mixture was heated at 100° C. for 12 h. The reaction mixture was partitioned between EtOAc and water. The aqueous layer was collected and acidified to pH 5-6 using 2N HCl. The mixture was extracted using 15% MeOH-dichloromethane. The combined organic layer was washed with ice cold brine, dried over anhydrous Na₂SO₄, and concentrated under reduced pressure. The product was purified using column chromatography to afford O-methyl-N-(2-oxo-4-(o-tolyl)-2H-chromen-7-yl) serine (305, 490 mg). LCMS (ESI) Calcd. for C₂₀H₁₉NO₅: 353, found [M+H]⁺=354.

Synthesis of 3-methoxy-2-((2-oxo-4-(o-tolyl)-2H-chromen-7-yl)amino)propenamide, 306 [Step 2]: An oven-dried round bottle flask charged with O-methyl-N-(2-oxo-4-(o-tolyl)-2H-chromen-7-yl) serine (305, 400 mg, 1.1 mmol) followed by DMF (2 mL) and THF (2 mL). HOBt (230 mg, 1.7 mmol), EDC·HCl (325 mg, 1.7 mmol), DIPEA (0.6 mL, 4.5 mmol) and ammonium bicarbonate (435 mg, 4.5 mmol) were added to the reaction mixture. The reaction mixture was stirred at ambient temperature for 17 h. The reaction mixture was partitioned between 15% MeOH-dichloromethane and water. The organic layer was collected, washed with brine, dried over anhydrous Na₂SO₄, and concentrated under reduced pressure. The product was purified using reverse phase prep-HPLC to afford 3-methoxy-2-((2-oxo-4-(o-tolyl)-2H-chromen-7-yl)amino)propanamide (306, 156 mg). LCMS (ESI) Calcd. for C₂₀H₂₀N₂O₄: 352, found [M+H]⁺=353.

Synthesis of chiral 3-methoxy-2-((2-oxo-4-phenyl-2H-chromen-7-yl)amino)propenamide, Example 198 and Example 199 [Step 3]: Two stereo isomers were separated from racemic 3-methoxy-2-((2-oxo-4-(o-tolyl)-2H-chromen-7-yl)amino)propanamide (306, 115 mg) using chiral PREP NP to afford Peak 1 as 3-methoxy-2-((2-oxo-4-phenyl-2H-chromen-7-yl)amino)propanamide (Example 198, 35 mg) and Peak 2 as 3-methoxy-2-((2-oxo-4-(o-tolyl)-2H-chromen-7-yl)amino)propanamide (Example 199, 28 mg). The absolute stereochemistry of these Examples was not determined.

Example 198: 3-methoxy-2-((2-oxo-4-phenyl-2H-chromen-7-yl)amino)propanamide: LCMS (ESI) Calcd. for C₂₀H₂₀N₂O₄: 352, found [M+H]⁺=353. ¹H NMR (400 MHz, DMSO-d₆) δ 7.40-7.32 (m, 3H), 7.21-7.18 (m, 1H), 7.13-7.06 (m, 2H), 6.89-6.59 (m, 3H), 6.50-6.49 (m, 1H), 5.86 (s, 1H), 4.14-4.12 (m, 1H), 3.66-3.65 (m, 2H), 3.32 (s, 3H), 2.14 (s, 3H).

Example 199: 3-methoxy-2-((2-oxo-4-(o-tolyl)-2H-chromen-7-yl)amino)propanamide: LCMS (ESI) Calcd. for C₂₀H₂₀N₂O₄: 352, found [M+H]⁺=353. ¹H NMR (400 MHz, DMSO-d₆): δ 7.42-7.30 (m, 3H), 7.20-7.05 (m, 3H), 6.69-6.67 (m, 1H), 6.62-6.59 (m, 2H), 6.49-6.48 (m, 1H), 5.85 (s, 1H), 4.15-4.10 (m, 1H), 3.65-3.63 (m, 2H), 3.32 (s, 3H), 2.14 (s, 3H).

NP Chiral PREP METHOD: Chiral separation was performed on an Agilent 1200 series instrument. Column name: CHIRALPAK IC (250×20 mm) 5μ. Operating at ambient temperature and flow rate is 18.0 mL/min. Mobile phase was mixture of 70% hexane, 15% ethyl acetate, 15% ethanol, held isocratic for up to 18 min. with detection at 358 nm wavelength.

### Example 200-201: Synthesis of 3-hydroxy-2-((2-oxo-4-(o-tolyl)-2H-chromen-7-yl)methyl)propanenitrile

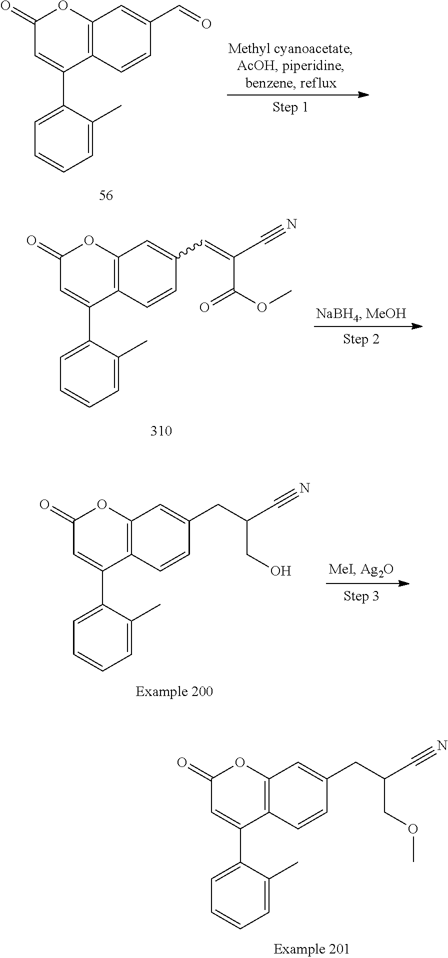 [SMILES: Cc1ccccc1-c1cc(=O)oc2cc(C=O)ccc12 ; COC(=O)C(C#N)=Cc1ccc2c(-c3ccccc3C)cc(=O)oc2c1 ; Cc1ccccc1-c1cc(=O)oc2cc(CC(C#N)CO)ccc12 ; COCC(C#N)Cc1ccc2c(-c3ccccc3C)cc(=O)oc2c1]

Synthesis of methyl-2-cyano-3-(2-oxo-4-(o-tolyl)-2H-chromen-7-yl)acrylate, 310 [Step 1]: To a stirred solution of 2-oxo-4-(o-tolyl)-2H-chromene-7-carbaldehyde (56, 700 mg, 2.6 mmol) in benzene (20 mL), methyl 2-cyanoacetate (0.2 mL, 2.7 mmol) and piperidine (0.03 mL, 0.3 mmol) were added and the reaction mixture was heated to reflux using Dean Stark apparatus at 90° C. for 16 h. TLC showed full conversion of starting material with formation of new polar spot. The reaction mixture was evaporated and the residue was purified by flash chromatography over silica gel to yield methyl-2-cyano-3-(2-oxo-4-(o-tolyl)-2H-chromen-7-yl)acrylate (310, 800 mg). LCMS (ESI) Calcd. for C₂₁H₁₅NO₄: 345, found [M+H]⁺=346.

Synthesis of 3-hydroxy-2-((2-oxo-4-(o-tolyl)-2H-chromen-7-yl)methyl)propanenitrile, Example 200 [Step 2]: To a stirred solution of methyl-2-cyano-3-[4-(o-tolyl)-2-oxo-chromen-7-yl]prop-2-enoate (310, 150 mg, 0.4 mmol) in methanol (5 mL), NaBH₄ (65 mg, 1.7 mmol) was added in ice cold condition and the reaction mixture was heated at 60° C. for 16 h. TLC showed full conversion of starting material with formation of new polar spot. The reaction mixture was diluted with EtOAc and washed with water and brine. The organic phase was dried over anhydrous Na₂SO₄, filtered and evaporated. The product was purified by reverse phase prep-HPLC and lyophilized to yield 3-hydroxy-2-((2-oxo-4-(o-tolyl)-2H-chromen-7-yl)methyl)propanenitrile (Example 200, 40 mg). LCMS (ESI) Calcd. for C₂₀H₁₇NO₃: 319, found [M H]⁺=320. ¹H NMR (400 MHz, DMSO-d₆) δ 7.47-7.34 (m, 4H), 7.28-7.22 (m, 2H), 6.94 (d, 1H), 6.36 (s, 1H), 5.42 (t, 1H), 3.63-3.52 (m, 2H), 3.26-3.17 (m, 1H), 3.05-2.93 (m, 2H), 2.12 (s, 3H).

Synthesis of 3-methoxy-2-((2-oxo-4-(o-tolyl)-2/1-chromen-7-yl)methyl)propanenitrile, Example 201 [Step 3]: In a sealed tube 3-hydroxy-2-((2-oxo-4-(o-tolyl)-2H-chromen-7-yl)methyl)propanenitrile (Example 200, 100 mg, 0.3 mmol) was taken in Mel (2.0 mL, 0.3 mmol). To the mixture Ag₂O (90 mg, 0.4 mmol) was added and stirred in dark at ambient temperature for 16 h. TLC showed full conversion of starting material with formation of new polar spot. The reaction mixture was diluted with EtOAc, filtered through celite bed, and evaporated. The product was purified by reverse phase prep-HPLC and lyophilized to yield 3-methoxy-2-((2-oxo-4-(o-tolyl)-2H-chromen-7-yl)methyl)propanenitrile (Example 201, 42 mg). LCMS (ESI) Calcd. for C₂₁H₁₉NO₃: 333, found [M+H]⁺: 334. ¹H NMR (400 MHz, DMSO-d₆ at 100° C.) δ 7.44-7.34 (m, 4H), 7.27-7.21 (m, 2H), 6.96 (d, 1H), 6.31 (s, 1H), 3.54-3.50 (m, 2H), 3.41-3.35 (m, 4H), 3.03 (br s, 2H), 2.14 (s, 3H).

### Example 202-203

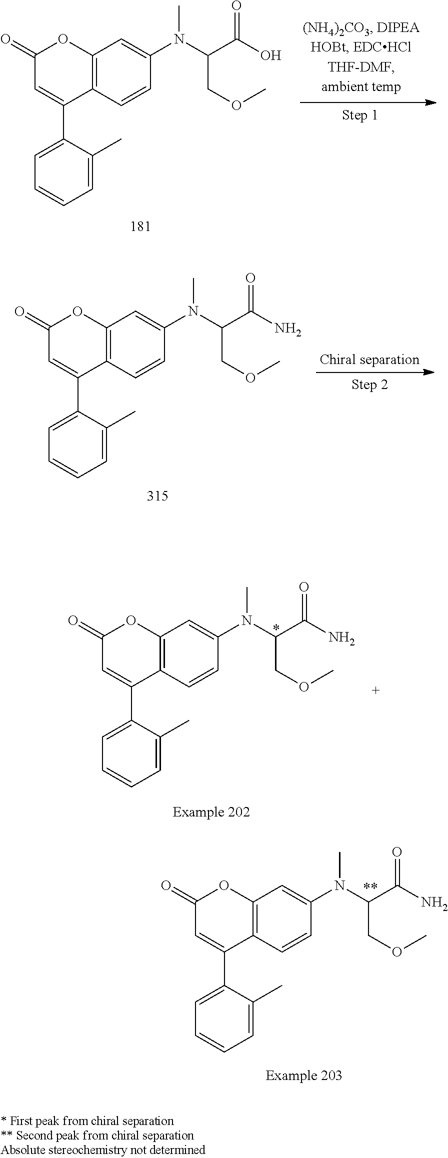 [SMILES: COCC(C(=O)O)N(C)c1ccc2c(-c3ccccc3C)cc(=O)oc2c1 ; COCC(C(N)=O)N(C)c1ccc2c(-c3ccccc3C)cc(=O)oc2c1 ; COCC(C(N)=O)N(C)c1ccc2c(-c3ccccc3C)cc(=O)oc2c1 ; COCC(C(N)=O)N(C)c1ccc2c(-c3ccccc3C)cc(=O)oc2c1]

Synthesis of 3-methoxy-2-(methyl(2-oxo-4-(o-tolyl)-2H-chromen-7-yl)amino)propenamide, 315 [Step 1]: An oven dried round bottle flask charged with N,O-dimethyl-N-(2-oxo-4-(o-tolyl)-2H-chromen-7-yl) serine (181, 500 mg, 1.1 mmol) followed by DMF (2 mL) and THF (2 mL). HOBt (220 mg, 1.6 mmol), EDC·HCl (315 mg, 1.6 mmol), DIPEA (0.6 mL, 4.4 mmol) and ammonium bicarbonate (420 mg, 4.4 mmol) were added to the reaction mixture. The reaction mixture was stirred at ambient temperature for 17 h. The reaction mixture was partitioned between 15% MeOH-dichloromethane and water. The organic layer was collected, washed with brine, dried over anhydrous Na₂SO₄, and concentrated under reduced pressure. The product was purified using reverse phase prep-HPLC and lyophilized to afford 3-methoxy-2-(methyl(2-oxo-4-(o-tolyl)-2H-chromen-7-yl)amino)propanamide (315, 75 mg). LCMS (ESI) Calcd. for C₂₁H₂₂N₂O₄: 366, found [M+H]⁺=367.

Synthesis of chiral 3-methoxy-2-(methyl(2-oxo-4-(o-tolyl)-2H-chromen-7-yl)amino)propenamide, Example 202 and Example 203 [Step 2]: Two isomers were separated from racemic 3-methoxy-2-(methyl(2-oxo-4-(o-tolyl)-2H-chromen-7-yl)amino)propanamide (315, 68 mg, 0.2 mmol) using chiral PREP NP purification to afford Peak 1 as 3-methoxy-2-(methyl(2-oxo-4-(o-tolyl)-2H-chromen-7-yl)amino)propanamide (Example 202, 20 mg) and Peak 2 as 3-methoxy-2-(methyl(2-oxo-4-(o-tolyl)-2H-chromen-7-yl)amino)propanamide (Example 203, 21 mg). The absolute stereochemistry of these Examples was not determined.

Example 202: 3-methoxy-2-(methyl(2-oxo-4-(o-tolyl)-2H-chromen-7-yl)amino)propanamide: LCMS (ESI) Calcd. for C₂₁H₂₂N₂O₄: 366, found [M+H]⁺=367. ¹H NMR (400 MHz, DMSO-d₆): δ 7.50 (s, 1H) 7.43-7.39 (m, 2H), 7.36-7.31 (m, 1H), 7.24-7.20 (m, 2H), 6.74-6.73 (m, 3H), 5.94 (s, 1H), 4.66-4.62 (m, 1H) 3.77-3.75 (m, 2H), 3.23-3.22 (m, 3H), 2.95 (s, 3H), 2.12 (s, 3H).

Example 203: 3-methoxy-2-(methyl(2-oxo-4-(o-tolyl)-2H-chromen-7-yl)amino)propanamide: LCMS (ESI) Calcd. for C₂₁H₂₂N₂O₄: 366, found [M+H]⁺=367. ¹H NMR (400 MHz, DMSO-d₆): δ 7.50 (s, 1H) 7.42-7.37 (m, 2H), 7.35-7.31 (m, 1H), 7.24-7.20 (m, 2H), 6.75-6.73 (m, 3H), 5.94 (s, 1H), 4.66-4.62 (m, 1H) 3.77-3.75 (m, 2H), 3.23-3.22 (m, 3H), 2.95 (s, 3H), 2.12 (s, 3H).

Chiral PREP (NP) METHOD: Chiral separation was performed on an Agilent 1200 series instrument. Column was a CHIRALPAK IG (250×21 mm) 5μ. Operating at ambient temperature and flow rate is 21.0 ml/min. Mobile phase was the mixture of 50% hexane, 25% dichloromethane and 25% ethanol, held this isocratic mixture for up to 22 min. with detection at 365 nm wavelength.

### Example 204: Synthesis of 7-((isoxazol-3-ylmethyl)(methyl)amino)-4-(o-tolyl)-2H-chromen-2-one

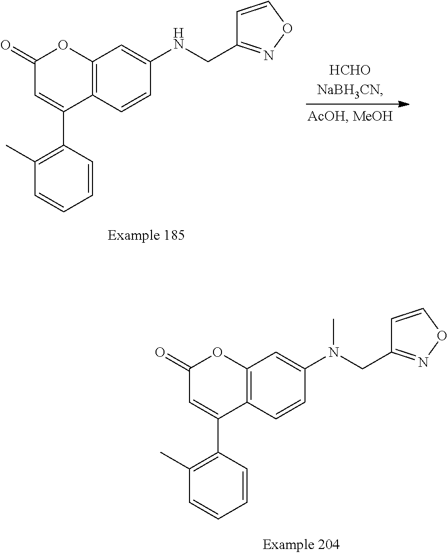 [SMILES: Cc1ccccc1-c1cc(=O)oc2cc(NCc3ccon3)ccc12 ; Cc1ccccc1-c1cc(=O)oc2cc(N(C)Cc3ccon3)ccc12]

Synthesis of 7-((isoxazol-3-ylmethyl)(methyl)amino)-4-(o-tolyl)-2H-chromen-2-one, Example 204: To a solution of 7-((isoxazol-3-ylmethyl)amino)-4-(o-tolyl)-2H-chromen-2-one (Example 185, 200 mg, 0.6 mmol) in methanol (4 mL) was added formaldehyde (37% in water) (0.4 mL, 0.9 mmol) followed by AcOH (catalytic amount) at ambient temperature and stirred for 16 h. at 40° C. Sodium cyanoborohydride (151 mg, 2.4 mmol) was then added and continue stirred at ambient temperature for 4 h. The reaction mixture was concentrated under reduced pressure, diluted with EtOAc, washed with water and brine, dried over anhydrous sodium sulfate, and concentrated under reduced pressure. The product was purified by reverse phase prep-HPLC and lyophilized to afford 7-((isoxazol-3-ylmethyl)(methyl)amino)-4-(o-tolyl)-2H-chromen-2-one (Example 204, 35 mg). LCMS (ESI) Calcd. for C₂₁H₁₈N₂O₃: 346, found [M+H]⁺=347. ¹H NMR (400 MHz, DMSO-d₆) δ 8.84 (s, 1H), 7.38-7.30 (m, 3H), 7.21-7.19 (d, 1H), 6.75 (m, 3H), 6.48 (s, 1H), 5.93 (s, 1H), 4.76 (s, 2H), 3.10 (s, 3H), 2.11 (s, 3H).

### Example 205-206: Synthesis of chiral N-methyl-N-((1-(2-oxo-4-(o-tolyl)-2H-chromene-7-carbonyl)pyrrolidin-3-yl)methyl)acetamide

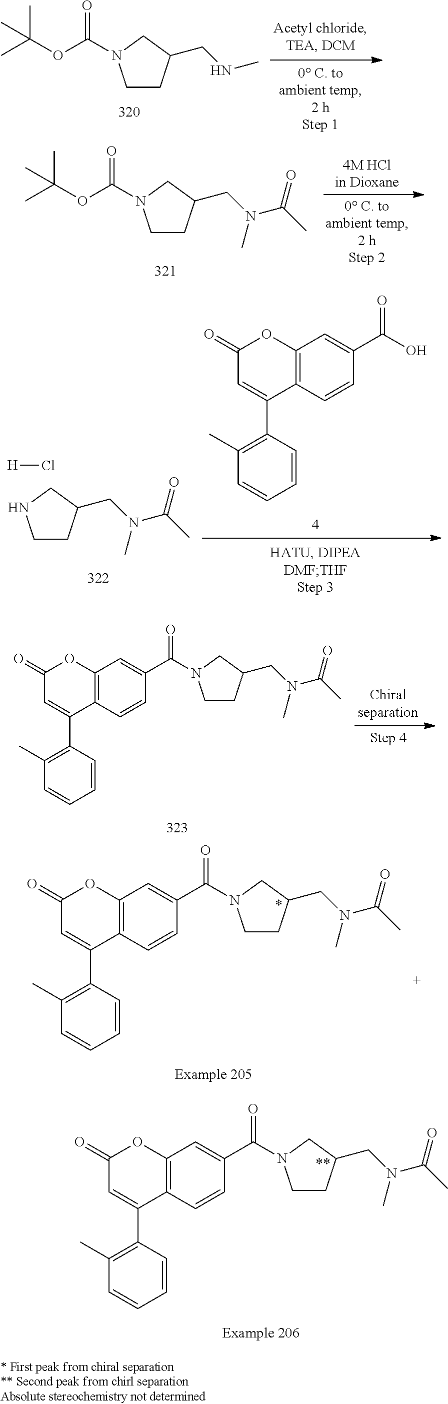

Synthesis of tert-butyl 3-((N-methylacetamido)methyl)pyrrolidine-1-carboxylate, 321 [Step 1]: To a stirred solution of tert-butyl 3-((methylamino)methyl)pyrrolidine-1-carboxylate (320, 250 mg, 1.17 mmol) in dichloromethane (1 mL), triethylamine (0.33 mL, 2.33 mmol) was added followed by addition of acetyl chloride (0.21 mL, 2.92 mmol) at 0° C. and stirring was continued for 2 h. The reaction mixture was then diluted with dichloromethane, partitioned with water and combined organic layer was collected, washed with brine, dried over anhydrous Na₂SO₄, filtered and concentrated under reduced pressure to afford the tert-butyl 3-((N-methylacetamido)methyl)pyrrolidine-1-carboxylate (321, 260 mg). ¹H NMR (400 MHz, DMSO-d₆) δ 3.31-3.16 (m, 4H), 2.95 (s, 2H), 2.92-2.90 (m, 1H), 2.78 (s, 1H), 2.45-2.40 (m, 2H), 1.98-1.82 (m, 4H), 1.54 (m, 1H), 1.39 (s, 9H).

Synthesis of N-methyl-N-(pyrrolidin-3-ylmethyl)acetamide hydrochloride, 322 [Step 2]: To a stirred solution of tert-butyl 3-((N-methylacetamido)methyl)pyrrolidine-1-carboxylate (321, 250 mg, 0.97 mmol) in dichloromethane 1 ml HCl in dioxane 4N (0.3 mL, 4.88 mmol) was added at 0° C. and stirring was continued for 2 h. The reaction mixture was concentrated under reduced pressure and lyophilized to afford N-methyl-N-(pyrrolidin-3-ylmethyl)acetamide as the HCl salt (322, 195 mg). It was used for the next step without further purification.

Synthesis of N-methyl-N-((1-(2-oxo-4-(o-tolyl)-2H-chromene-7-carbonyl)pyrrolidin-3-yl)methyl)acetamide, 323 [Step 3]: To a solution of 2-oxo-4-(o-tolyl)-2H-chromene-7-carboxylic acid (4, 250 mg, 0.892 mmol) in DMF (1.5 mL), HATU (508 mg, 1.34 mmol) was added followed by addition of N,N-Diisopropylethylamine (3.00 eq, 0.47 mL, 2.68 mmol) and the reaction mixture was stirred for 15 minutes at ambient temperature under nitrogen balloon. Then N-methyl-N-(pyrrolidin-3-ylmethyl)acetamide (HCl salt) (190 mg, 1.34 mmol) was added to the reaction mixture and stirring was continued for 16 h. at ambient temperature. After completion of the reaction, the reaction mixture was diluted with EtOAc and washed with 5% aqueous K₂CO₃ solution. The organic phase was then washed with ice cold water and brine. The organic phase was dried over anhydrous Na₂SO₄, filtered and evaporated. The product was purified through reverse phase prep-HPLC and lyophilized to afford N-methyl-N-((1-(2-oxo-4-(o-tolyl)-2H-chromene-7-carbonyl)pyrrolidin-3-yl)methyl)acetamide (323, 150 mg). LCMS (ESI) Calcd. For C₂₅H₂₆N₂O₄: 418, found [M+H]⁺=419.1H NMR (400 MHz, DMSO-d₆ at 100° C.) δ 7.55 (s, 1H), 7.47-7.35 (m, 4H), 7.29 (d, 1H), 7.05 (d, 1H), 6.39 (s, 1H), 3.57-3.47 (m, 4H), 3.34 (s, 2H), 3.19 (br s, 1H), 2.97-2.50 (m, 3H), 2.16 (s, 3H), 1.98 (s, 4H), 1.67-1.62 (m, 1H).

Synthesis of chiral N-methyl-N-((1-(2-oxo-4-(o-tolyl)-2H-chromene-7-carbonyl)pyrrolidin-3-yl)methyl)acetamide, Example 205 and Example 206 [Step 4]: N-methyl-N-((1-(2-oxo-4-(o-tolyl)-2H-chromene-7-carbonyl)pyrrolidin-3-yl)methyl)acetamide (323, 120 mg) was further purified via SFC purification and lyophilized to afford Peak 1 as N-methyl-N-((1-(2-oxo-4-(o-tolyl)-2H-chromene-7-carbonyl)pyrrolidin-3-yl)methyl)acetamide (Example 205, 27 mg) and Peak 2 as N-methyl-N-((1-(2-oxo-4-(o-tolyl)-2H-chromene-7-carbonyl)pyrrolidin-3-yl)methyl)acetamide (Example 206, 22 mg). The absolute stereochemistry of these Examples was not determined.

Example 205: N-methyl-N-((1-(2-oxo-4-(o-tolyl)-2H-chromene-7-carbonyl)pyrrolidin-3-yl)methyl)acetamide, Peak 1: LCMS (ESI) Calcd. for C₂₅H₂₆N₂O₄: 418, found [M+H]⁺=419.1H NMR (400 MHz, DMSO-d₆ at 100° C.) δ 7.55 (s, 1H), 7.47-7.35 (m, 4H), 7.29 (d, 1H), 7.05 (d, 1H), 6.39 (s, 1H), 3.57-3.47 (m, 4H), 3.34 (s, 2H), 3.19 (br s, 1H), 2.97-2.50 (m, 3H), 2.16 (s, 3H), 1.98 (s, 4H), 1.67-1.62 (m, 1H).

Example 206: N-methyl-N-((1-(2-oxo-4-(o-tolyl)-2H-chromene-7-carbonyl)pyrrolidin-3-yl)methyl)acetamide, Peak 2: LCMS (ESI) Calcd. for C₂₅H₂₆N₂O₄: 418, found [M+H]⁺=419.1H NMR (400 MHz, DMSO-d₆) δ (at 100° C.) 7.55 (s, 1H), 7.47-7.35 (m, 4H), 7.29 (d, 1H), 7.05 (d, 1H), 6.39 (s, 1H), 3.57-3.47 (m, 4H), 3.34 (s, 2H), 3.19 (br s, 1H), 2.97-2.50 (m, 3H), 2.16 (s, 3H), 1.98 (s, 4H), 1.67-1.62 (m, 1H).

Chiral prep SFC purification method: Chiral separation was performed on a Thar SFC-80 series instrument using REGIS Reflect (R,R) WHELK-01 column (250×21.1 mm), 5μ, operating at 35° C., and maintaining a flow rate of 60 gm/min, using 80% CO₂ in super critical state and 20% of 0.3% isopropylamine in EtOH/MeOH (70/30) as mobile phase, held isocratic for up to 12 min. and also maintained the isobaric condition of 110 bar with detection at 220 nm wavelength.

### Example 207-208: Synthesis of chiral 3-methoxy-2-((2-oxo-4-(o-tolyl)-2H-chromen-7-yl)methyl)propenamide

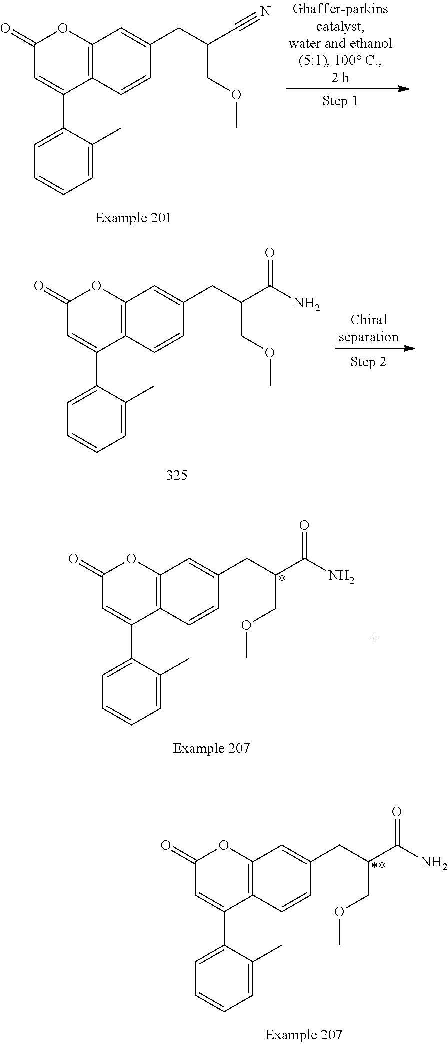 [SMILES: COCC(C#N)Cc1ccc2c(-c3ccccc3C)cc(=O)oc2c1 ; COCC(Cc1ccc2c(-c3ccccc3C)cc(=O)oc2c1)C(N)=O ; COCC(Cc1ccc2c(-c3ccccc3C)cc(=O)oc2c1)C(N)=O ; COCC(Cc1ccc2c(-c3ccccc3C)cc(=O)oc2c1)C(N)=O]

Synthesis of 3-methoxy-2-((2-oxo-4-(o-tolyl)-2/I-chromen-7-yl)methyl)propenamide, 325 [Step 1]: To a stirred solution of 3-methoxy-2-((2-oxo-4-(o-tolyl)-2H-chromen-7-yl)methyl)propanenitrile (Example 201, 300 mg, 0.9 mmol) in ethanol (2 mL) and water (10 mL) mixture, nitrogen was purged for 5 min. Then Ghaffer-perkin catalyst (3.9 mg, 0.009 mmol) was added and the purging was continued for 5 min. The reaction mixture was heated at 100° C. for 2 h. The reaction mixture was cooled and extracted with ethyl acetate. The organic phase was washed with brine, dried over anhydrous Na₂SO₄, filtered and evaporated. The product was purified by reverse phase prep-HPLC and lyophilized to yield 3-methoxy-2-((2-oxo-4-(o-tolyl)-2H-chromen-7-yl)methyl)propanamide (325, 150 mg). LCMS (ESI) Calcd. for C₂₁H₂₁NO₄: 351, found [M+H]⁺=352.

Synthesis of chiral 3-methoxy-2-((2-oxo-4-(o-tolyl)-2H-chromen-7-yl)methyl)propenamide, Example 207 and Example 208 [Step 2]: The racemic compound 3-methoxy-2-((2-oxo-4-(o-tolyl)-2H-chromen-7-yl)methyl)propanamide (325, 150 mg) was submitted for SFC chiral prep purification. The fractions obtained were lyophilized to yield Peak 1 as 3-methoxy-2-((2-oxo-4-(o-tolyl)-2H-chromen-7-yl)methyl)propanamide (Example 207, 52 mg) and Peak 2 as 3-methoxy-2-((2-oxo-4-(o-tolyl)-2H-chromen-7-yl)methyl)propanamide (Example 208, 30 mg). The absolute stereochemistry of these Examples was not determined.

Example 207: 3-methoxy-2-((2-oxo-4-(o-tolyl)-2H-chromen-7-yl)methyl)propenamide Peak 1: LCMS (ESI) Calcd. for C₂₁H₂₁NO₄: 351, found [M+H]⁺=352. ¹H NMR (400 MHz, DMSO-d₆) at 100° C. δ 7.46-7.30 (m, 4H), 7.25 (d, 1H), 7.12 (d, 1H), 6.90 (d, 1H), 6.70 (br s, 2H), 6.25 (s, 1H), 3.54-3.48 (m, 1H), 3.39-3.35 (m, 1H), 3.26 (s, 3H), 2.92-2.79 (m, 3H), 2.14 (s, 3H).

Example 208: 3-methoxy-2-((2-oxo-4-(o-tolyl)-2H-chromen-7-yl)methyl)propenamide Peak 2: LCMS (ESI) Calcd. for C₂₁H₂₁NO₄: 351, found [M+H]⁺=352. ¹H NMR (400 MHz, CDCl₃) δ 7.41-7.28 (m, 4H), 7.16 (d, 1H), 7.04-6.96 (m, 2H), 6.27 (s, 1H), 6.17 (br s, 1H), 5.30 (br s, 1H), 3.51-3.41 (m, 2H), 3.36 (s, 3H), 3.16-3.11 (m, 1H), 2.89-2.84 (m, 1H), 2.70 (br s, 1H), 2.15 (s, 3H).

Prep SFC method: Column was a C-AMYLOSE-A (30×250 mm), 5μ, with a flow of 60 g/min and a mobile phase of 60% CO₂ and 40% (0.2% isopropylamine in MeOH) with a pressure of 80 bar at 35° C. using a wavelength of 215 nm.

### Example 209: Synthesis of 5-((methyl(2-oxo-4-(o-tolyl)-2H-chromen-7-yl)amino)methyl)pyridin-2 (1H)-one

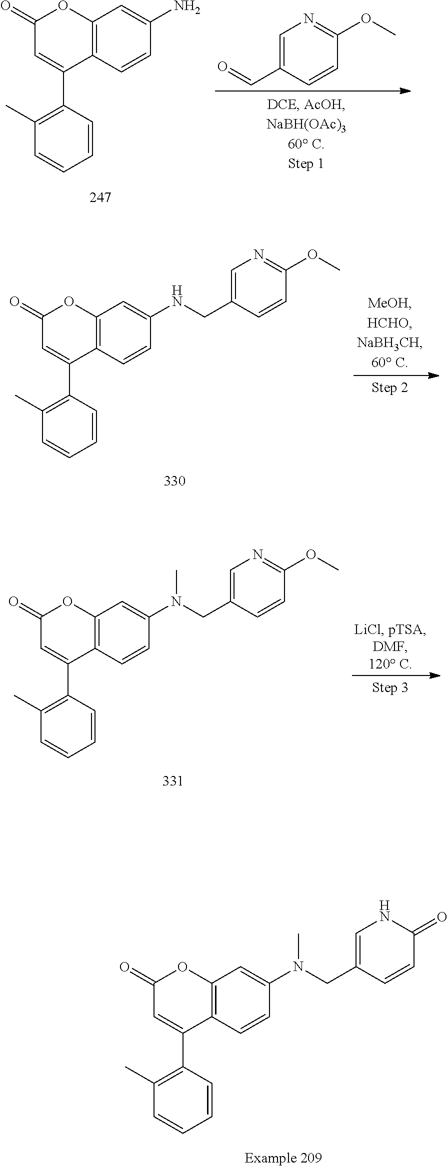

Synthesis of 7-(((6-methoxypyridin-3-yl)methyl)amino)-4-(o-tolyl)-2H-chromen-2-one, 330 [Step 1]: To a solution of 7-amino-4-(o-tolyl)-2H-chromen-2-one (247, 500 mg, 2.1 mmol) in DCE (10 mL) was added 6-methoxynicotinaldehyde (565 mg, 4.2 mmol) and AcOH (1 mL, 2.1 mmol) at ambient temperature and stirred at 60° C. for 16 h. NaBH(OAc)₃ (1.5 g, 8.4 mmol) was added and stirring was continued at 60° C. for 4 h. The reaction mixture was quenched with saturated NH₄Cl solution and extracted with EtOAc. The combined organic layer was dried over anhydrous Na₂SO₄, and concentrated under reduced pressure to afford 7-(((6-methoxypyridin-3-yl)methyl)amino)-4-(o-tolyl)-2H-chromen-2-one (330, 975 mg). LCMS (ESI) Calcd. for C₂₃H₂₀N₂O₃: 372, found [M+H]⁺=373.

Synthesis of 7-(((6-methoxypyridin-3-yl)methyl)(methyl)amino)-4-(o-tolyl)-2H-chromen-2-one, 331 [Step 2]: To a solution of 7-(((6-methoxypyridin-3-yl)methyl)amino)-4-(o-tolyl)-2H-chromen-2-one (330, 400 mg, 1.1 mmol) in methanol (5 mL) was added formaldehyde (0.1 mL, 1.6 mmol) and AcOH (0.1 mL, 1.1 mmol) at ambient temperature and heated at 60° C. for 16 h. After completion the reaction mixture was cooled to ambient temperature and NaBH₃CN (270 mg, 4.3 mmol) was added and further stirred for 4 h. The reaction mixture was quenched with saturated NH₄Cl solution and extracted with EtOAc. The combined organic layer was dried over anhydrous Na₂SO₄ and concentrated under reduced pressure to afford 7-(((6-methoxypyridin-3-yl)methyl)(methyl)amino)-4-(o-tolyl)-2H-chromen-2-one (331, 410 mg). LCMS (ESI) Calcd. for C₂₄H₂₂N₂O₃:386, found [M+H]⁺=387.

Synthesis of 5-((methyl(2-oxo-4-(o-tolyl)-2H-chromen-7-yl)amino)methyl)pyridin-2 (1H)-one, Example 209 [Step 3]: To a solution of 7-(((6-methoxypyridin-3-yl)methyl)(methyl)amino)-4-(o-tolyl)-2H-chromen-2-one (331, 400 mg, 0.2 mmol) in DMF (4 mL) was added LiCl (25 mg, 0.6 mmol) and pTSA·H₂O (125 mg, 0.6 mmol) and heated at 120° C. for 4 h. After completion, the reaction mixture was concentrated under reduced pressure and purified by reverse phase prep-HPLC to afford 5-((methyl(2-oxo-4-(o-tolyl)-2H-chromen-7-yl)amino)methyl)pyridin-2 (1H)-one (Example 209, 7 mg). LCMS (ESI) Calcd. for C₂₃H₂₀N₂O₃: 372, found [M+H]⁺=373. ¹H NMR (400 MHz, DMSO-d₆) δ 11.46 (s, 1H), 7.40-7.38 (m, 2H), 7.33-7.30 (m, 2H), 7.25 (s, 1H), 7.20 (d, 1H), 6.74 (t, 3H), 6.30 (d, 1H), 5.91 (s, 1H), 4.40 (s, 2H), 3.05 (s, 3H), 2.11 (s, 3H).

### Example 210: Synthesis of 2-methyl-2-((2-oxo-4-(o-tolyl)-2H-chromen-7-yl)amino)propanamide

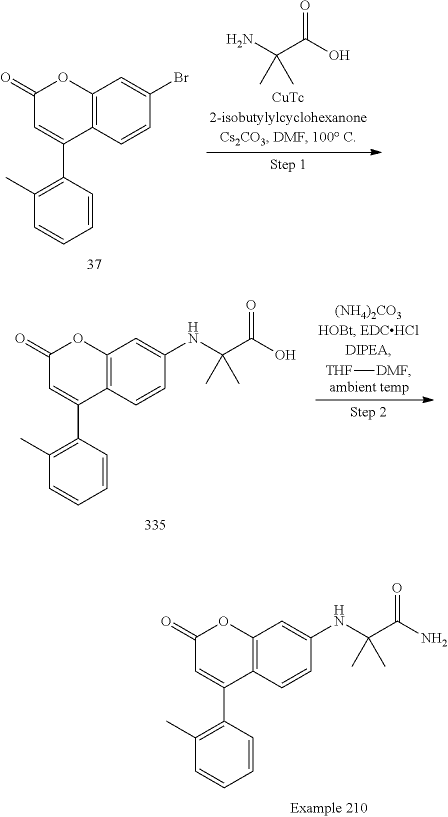

Synthesis of 2-methyl-2-((2-oxo-4-(o-tolyl)-2H-chromen-7-yl)amino)propanoic acid, 335 [Step 1]: A sealed tube was charged with 7-bromo-4-(o-tolyl)-2H-chromen-2-one (37, 200 mg, 0.6 mmol), 2-amino-2-methylpropanoic acid (100 mg, 0.9 mmol), copper thiophene-2-carboxylate (25 mg, 0.1 mmol), 2-isobutyrylcyclohexanone (0.1 mL, 0.5 mmol), Cs₂CO₃ (415 mg, 1.3 mmol) and DMF (6 mL). The reaction vessel was flushed with argon gas, and then capped tightly. The reaction mixture was heated at 100° C. for 16 h. After completion, the reaction mixture was quenched with 1N aqueous HCl, and extracted with 20% MeOH-dichloromethane. The combined organic phase was dried over anhydrous Na₂SO₄, filtered and concentrated under reduced pressure to afford 2-methyl-2-((2-oxo-4-(o-tolyl)-2H-chromen-7-yl)amino)propanoic acid (335, 250 mg). LCMS (ESI) Calcd. for C₂₀H₁₉NO₄: 337, found [M+H]⁺=338.

Synthesis of 2-methyl-2-((2-oxo-4-(o-tolyl)-2H-chromen-7-yl)amino)propanamide, Example 210 [Step 2]: To a stirred solution of 2-methyl-2-((2-oxo-4-(o-tolyl)-2H-chromen-7-yl)amino)propanoic acid (335, 250 mg, 0.7 mmol) in DMF-THF (4 mL, 1:1) mixture, HOBt (150 mg, 1.1 mmol), EDC·HCl (215 mg, 1.1 mmol), DIPEA (0.4 mL, 3.0 mmol) and (NH₄)₂CO₃ (285 mg, 3.0 mmol) were added successively and the mixture was stirred at ambient temperature for 16 h. The reaction was quenched with water and extracted with 20% MeOH in dichloromethane. The combined organic phase was dried over anhydrous Na₂SO₄, filtered, and concentrated under reduced pressure. The product was purified by reverse phase prep-HPLC to afford 2-methyl-2-((2-oxo-4-(o-tolyl)-2H-chromen-7-yl)amino)propanamide (Example 210, 150 mg). LCMS (ESI) Calcd. for C₂₀H₂₀N₂O₃: 336, found [M+H]⁺=337. ¹H NMR (400 MHz, DMSO-d₆): δ 7.42-7.30 (m, 4H), 7.19 (d, 1H), 7.07 (br s, 1H), 6.86 (br s, 1H), 6.65 (d, 1H), 6.50 (dd, 1H), 6.32 (d, 1H), 5.88 (s, 1H), 2.11 (s, 3H), 1.39 (s, 6H).

### Example 211: Synthesis of (R)-2-((2-oxo-4-(o-tolyl)-2H-chromen-7-yl)amino)butanamide

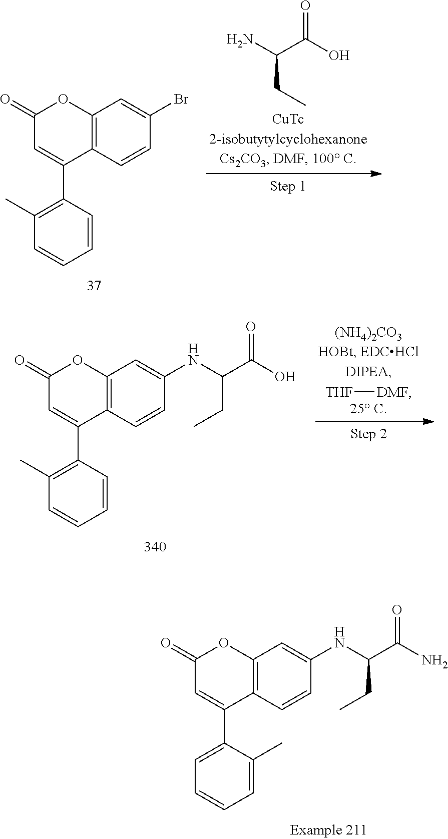

Synthesis of 2-((2-oxo-4-(o-tolyl)-2H-chromen-7-yl)amino)butanoic acid, 340 [Step 1]: A sealed tube was charged with 7-bromo-4-(o-tolyl)-2H-chromen-2-one (37, 300 mg, 1.0 mmol), (S)-2-aminobutanoic acid (120 mg, 1.1 mmol), copper thiophene 2-carboxylate (35 mg, 0.2 mmol), 2-isobutyrylcyclohexanone (0.1 mL, 0.8 mmol), Cs₂CO₃ (620 mg, 1.9 mmol) and DMF (5 mL). The reaction vessel was flushed with argon gas and was tightly capped. The reaction tube was put in a heating block preheated to 100° C. and stirred for 12 h. After cooling to ambient temperature, the reaction mixture was acidified with 1N aq. HCl to pH=2 and extracted with 20% MeOH-dichloromethane. The combined organic phase was dried over anhydrous Na₂SO₄, filtered and concentrated under reduced pressure to afford 2-((2-oxo-4-(o-tolyl)-2H-chromen-7-yl)amino)butanoic acid (340, 300 mg). LCMS (ESI) Calcd. for C₂₀H₁₉NO₄: 337, found [M+H]⁺=338.

Synthesis of (R)-2-((2-oxo-4-(o-tolyl)-2H-chromen-7-yl)amino)butanamide, Example 211 [Step 2]: To a stirred solution of 2-((2-oxo-4-(o-tolyl)-2H-chromen-7-yl)amino)butanoic acid (340, 300 mg, 0.9 mmol) in DMF (2 mL)-THF (2 mL) mixture, was successively added HOBt (180 mg, 1.3 mmol), EDC·HCl (255 mg, 1.3 mmol), DIPEA (0.5 mL, 3.6 mmol) and (NH₄)₂CO₃ (340 mg, 3.6 mmol), and the mixture was stirred at ambient temperature. After 16 h., the reaction was quenched with water, and extracted with 20% MeOH in dichloromethane. The combined organic phase was dried over anhydrous Na₂SO₄, filtered, and concentrated under reduced pressure. The product was purified by reverse phase prep-HPLC and isolated the major isomer by Chiral-HPLC (NP) to afford (R)-2-((2-oxo-4-(o-tolyl)-2H-chromen-7-yl)amino)butanamide (Example 211, 50 mg). LCMS (ESI) Calcd. for C₂₀H₂₀N₂O₃: 336, found [M+H]⁺=337. ¹H NMR (400 MHz) δ 7.53-7.52 (m, 1H), 7.40-7.32 (m, 3H), 7.20-7.18 (m, 1H), 7.11 (s, 1H), 6.84-6.82 (m, 1H), 6.66-6.63 (m, 1H), 6.60-6.59 (m, 1H) 6.49 (s, 1H), 5.86 (s, 1H), 3.77-3.76 (m, 1H), 2.11 (s, 3H), 1.75-1.68 (m, 2H), 0.96-0.92 (m, 3H).

Chiral PREP method: Chiral separation was performed on an Agilent 1200 series instrument. Column was a CHIRALPAK IG (250×21 mm) 5μ. Operating at ambient temperature and flow rate is 21.0 mL/min. Mobile phase was mixture of 60% hexane, 20% DCM, 20% EtOH, held this isocratic mixture for up to 25 min. with detection at 360 nm wavelength.

### Example 212: Synthesis of (S)-2-((2-oxo-4-(o-tolyl)-2H-chromen-7-yl)amino)butanamide

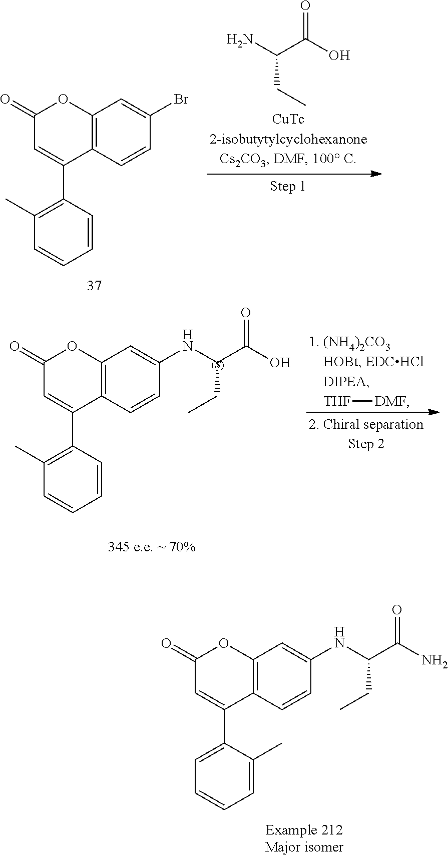

Synthesis of (S)-2-((2-oxo-4-(o-tolyl)-2H-chromen-7-yl)amino)butanoic acid, 345 [Step 1]: A sealed tube was charged with 7-bromo-4-(o-tolyl)-2H-chromen-2-one (37, 300 mg, 1.0 mmol), (S)-2-aminobutanoic acid (120 mg, 1.1 mmol), copper thiophene 2-carboxylate (35 mg, 0.2 mmol), 2-isobutyrylcyclohexanone (0.1 mL, 0.8 mmol), Cs₂CO₃ (620 mg, 1.9 mmol) and DMF (5 mL). The reaction vessel was flushed with argon gas and was tightly capped. The reaction tube was put in a heating block preheated to 100° C. and was stirred for 12 h. After cooling to ambient temperature, the reaction mixture was acidified with 1N aq. HCl to pH 2 and extracted with 20% MeOH-dichloromethane. The combined organic phase was dried over anhydrous Na₂SO₄, filtered, and concentrated under reduced pressure to afford(S)-2-((2-oxo-4-(o-tolyl)-2H-chromen-7-yl)amino)butanoic acid (345, 300 mg). LCMS (ESI) Calcd. for C₂₀H₁₉NO₄: 337, found [M+H]⁺=338.

Synthesis of (S)-2-((2-oxo-4-(o-tolyl)-2H-chromen-7-yl)amino)butanamide, Example 212 [Step 2]: To a stirred solution of (S)-2-((2-oxo-4-(o-tolyl)-2H-chromen-7-yl)amino)butanoic acid (345, 300 mg, 0.9 mmol) in DMF (2.5 mL)-THF (2.5 mL) mixture, was successively added HOBt (180 mg, 1.3 mmol), EDC·HCl (255 mg, 1.3 mmol), DIPEA (0.5 mL, 3.6 mmol) and (NH₄)₂CO₃ (340 mg, 3.6 mmol), and the reaction mixture was stirred at ambient temperature. After 16 h., the reaction was quenched with water, and extracted with 20% MeOH in dichloromethane. The combined organic phase was dried over anhydrous Na₂SO₄, filtered, and concentrated under reduced pressure. The product was purified by reverse phase prep-HPLC and isolated the major isomer by Chiral-HPLC NP to afford(S)-2-((2-oxo-4-(o-tolyl)-2H-chromen-7-yl)amino)butanamide (Example 212, 68 mg). LCMS (ESI) Calcd. for C₂₀H₂₀N₂O₃: 336, found [M+H]⁺=337. ¹H NMR (400 MHz) δ 7.54-7.53 (m, 1H), 7.40-7.31 (m, 3H), 7.20-7.18 (m, 1H), 7.11 (s, 1H), 6.84-6.82 (m, 1H), 6.66-6.63 (m, 2H), 6.49 (s, 1H), 5.86 (s, 1H), 3.77-3.76 (m, 1H), 2.11 (s, 3H), 1.75-1.68 (m, 2H), 0.96-0.92 (m, 3H).

Chiral separation method: Chiral separation was performed on an Agilent 1200 series instrument. Column was a CHIRALPAK IG (250×21 mm) 5μ. Operating at ambient temperature and flow rate is 21.0 mL/min. Mobile phase was mixture of 60% hexane, 20% DCM, 20% ethyl alcohol, held this isocratic mixture for up to 30 min. with detection at 360 nm wavelength.

### Example 213: Synthesis of 1-((2-oxo-4-(o-tolyl)-2H-chromen-7-yl)amino)cyclopropane-1-carboxamide

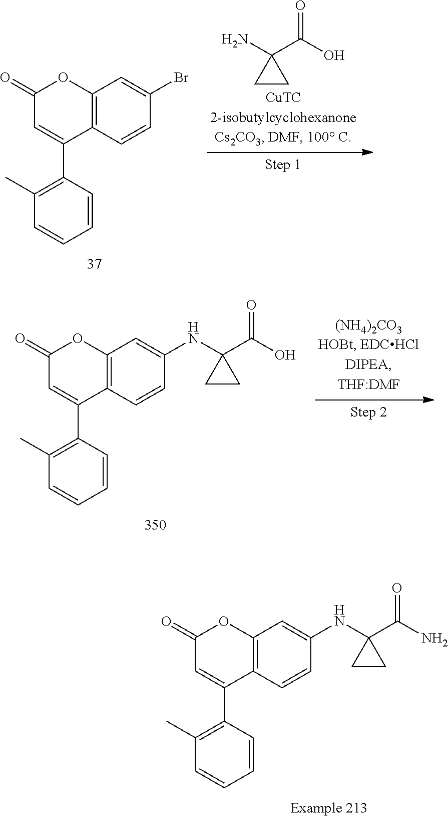 [SMILES: Cc1ccccc1-c1cc(=O)oc2cc(Br)ccc12 ; NC1(C(=O)O)CC1 ; Cc1ccccc1-c1cc(=O)oc2cc(NC3(C(=O)O)CC3)ccc12 ; Cc1ccccc1-c1cc(=O)oc2cc(NC3(C(N)=O)CC3)ccc12]

Synthesis of 1-((2-oxo-4-(o-tolyl)-2H-chromen-7-yl)amino)cyclopropane-1-carboxylic acid, 350 [Step 1]: A sealed tube was charged with 7-bromo-4-(o-tolyl)-2H-chromen-2-one (37, 200 mg, 0.6 mmol), 1-aminocyclopropane-1-carboxylic acid (95 mg, 0.9 mmol), copper thiophene 2-carboxylate (25 mg, 0.1 mmol), 2-isobutyrylcyclohexanone (0.1 mL, 0.5 mmol), Cs₂CO₃ (415 mg, 1.3 mmol) and DMF (4 mL). The reaction vessel was flushed with argon gas and was tightly capped. The reaction tube was put in a heating block preheated to 100° C. and stirred for 16 h. After cooling to ambient temperature, the reaction mixture was acidified with 1N aq. HCl to pH 2 and extracted with 20% MeOH-dichloromethane. The combined organic phase was dried over anhydrous Na₂SO₄, filtered, and concentrated under reduced pressure to afford 1-((2-oxo-4-(o-tolyl)-2H-chromen-7-yl)amino)cyclopropane-1-carboxylic acid (350, 200 mg). LCMS (ESI) Calcd. for C₂₀H₁₇NO₄: 335, found [M+H]⁺=336.

Synthesis of 1-((2-oxo-4-(o-tolyl)-2H-chromen-7-yl)amino)cyclopropane-1-carboxamide, Example 213 [Step 2]: To a stirred solution of 1-((2-oxo-4-(o-tolyl)-2H-chromen-7-yl)amino)cyclopropane-1-carboxylic acid (350, 250 mg, 0.7 mmol) in DMF (2 mL)-THF (2 mL) mixture, was successively added HOBt (151 mg, 1.1 mmol), EDC·HCl (214 mg, 1.1 mmol), DIPEA (0.4 mL, 3.0 mmol) and (NH₄)₂CO₃ (287 mg, 3.0 mmol) and the reaction mixture was stirred at ambient temperature. After 16 h., the reaction was quenched with water, and extracted with 20% MeOH in dichloromethane. The combined organic phase was dried over anhydrous Na₂SO₄, filtered, and concentrated under reduced pressure. The compound was purified by reverse phase prep-HPLC to afford 1-((2-oxo-4-(o-tolyl)-2H-chromen-7-yl)amino)cyclopropane-1-carboxamide (Example 213, 68 mg). LCMS (ESI) Calcd. for C₂₀H₁₈N₂O₃: 334, found [M+H]⁺=335. ¹H NMR (400 MHz, DMSO-d₆): δ 7.42-7.36 (m, 3H), 7.34-7.30 (m, 2H), 7.20-7.18 (m, 1H), 7.14 (br s, 1H), 6.70 (d, 1H), 6.53 (dd, 1H), 6.46 (br s, 1H), 5.92 (s, 1H), 2.12 (s, 3H), 1.40 (br s, 2H), 0.91 (br s, 2H).

### Example 216: Synthesis of 1-(((2-oxo-4-(o-tolyl)-2H-chromen-7-yl)amino)methyl)cyclopropane-1-carboxylic acid

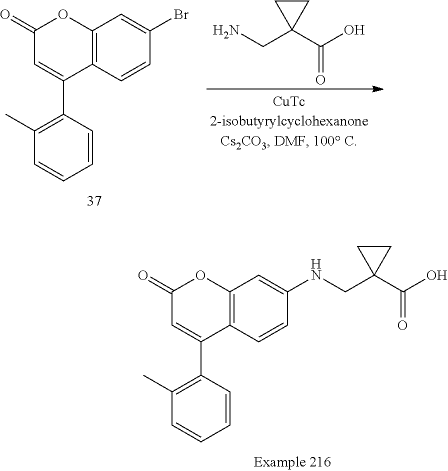 [SMILES: Cc1ccccc1-c1cc(=O)oc2cc(Br)ccc12 ; NCC1(C(=O)O)CC1 ; Cc1ccccc1-c1cc(=O)oc2cc(NCC3(C(=O)O)CC3)ccc12]

Synthesis of 1-(((2-oxo-4-(o-tolyl)-2H-chromen-7-yl)amino)methyl)cyclopropane-1-carboxylic acid, Example 216: A sealed tube was charged with 1-(aminomethyl)cyclopropane-1-carboxylic acid (90 mg, 0.8 mmol), 7-bromo-4-(o-tolyl)-2H-chromen-2-one (37, 200 mg, 0.6 mmol), copper thiophene 2-carboxylate (25 mg, 0.1 mmol), 2-isobutyrylcyclohexanone (0.1 mL, 0.5 mmol), cesium carbonate (410 mg, 1.3 mmol) and DMF (1.5 mL). The reaction vessel was flushed with argon gas, and immersed into a heating block preheated to 100° C. After 16 h., the reaction mixture was acidified using 2N HCl until pH 5-6, and extracted with 20% MeOH in DCM (twice). The combined organic layer was dried over anhydrous sodium sulfate, filtered, and concentrated under reduced pressure. The product was purified by RP prep-HPLC and lyophilized to afford 1-(((2-oxo-4-(o-tolyl)-2H-chromen-7-yl)amino)methyl)cyclopropane-1-carboxylic acid (Example 216, 85 mg). LCMS (ESI) Calcd. for C₂₁H₁₉NO₄: 349, found [M+H]⁺=350. ¹H NMR (400 MHz, methanol-d₄): δ 7.38-7.28 (m, 3H), 7.18-7.16 (m, 1H), 6.74-6.72 (m, 1H), 6.54-6.52 (m, 2H), 5.85 (s, 1H), 3.41 (s, 2H), 2.15 (s, 3H), 1.25 (s, 2H), 0.94 (s, 2H).

### Example 217: Synthesis of 1-((methyl(2-oxo-4-(o-tolyl)-2H-chromen-7-yl)amino)methyl)cyclopropane-1-carboxylic acid

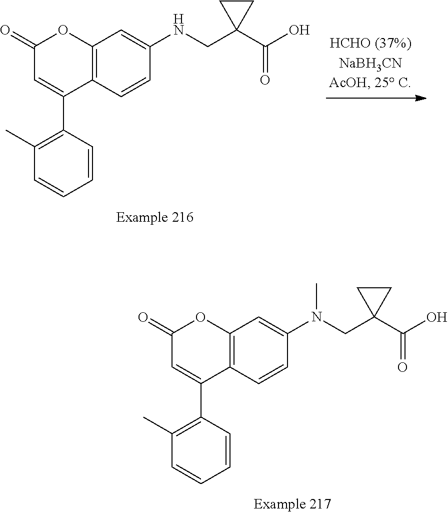 [SMILES: Cc1ccccc1-c1cc(=O)oc2cc(NCC3(C(=O)O)CC3)ccc12 ; Cc1ccccc1-c1cc(=O)oc2cc(N(C)CC3(C(=O)O)CC3)ccc12]

Synthesis of 1-((methyl(2-oxo-4-(o-tolyl)-2H-chromen-7-yl)amino)methyl)cyclopropane-1-carboxylic acid, Example 217: To a stirred solution of 1-(((2-oxo-4-(o-tolyl)-2H-chromen-7-yl)amino)methyl)cyclopropane-1-carboxylic acid (Example 216, 65 mg, 0.2 mmol) in acetic acid (0.5 mL) was added formaldehyde, 37% aqueous solution (0.1 mL, 1.9 mmol) and sodium cyanoborohydride (35 mg, 0.6 mmol) sequentially at 25° C. and stirred for 1 h. The reaction mixture was quenched with water, and extracted with 10% MeOH in dichloromethane (twice). The combined organic layer was dried over anhydrous Na₂SO₄ and concentrated under reduced pressure. The product was purified by prep-HPLC and lyophilized to afford 1-((methyl(2-oxo-4-(o-tolyl)-2H-chromen-7-yl)amino)methyl)cyclopropane-1-carboxylic acid (Example 217, 25 mg). LCMS (ESI) Calcd. for C₂₂H₂₁NO₄ 363, found [M+H]⁺: 364. ¹H NMR (400 MHz, DMSO-d₆): δ 12.29 (br s, 1H), 7.43-7.31 (m, 3H), 7.22-7.20 (m, 1H), 6.72-6.66 (m, 3H), 5.90 (s, 1H), 3.80 (m, 2H), 2.98 (s, 3H), 2.12 (s, 3H), 1.11-1.03 (m, 2H), 0.80-0.79 (m, 2H).

### Example 218-221: Synthesis of N-(2-methoxyethyl)-2-(methyl(2-oxo-4-(o-tolyl)-2H-chromen-7-yl)amino)acetamide, N-(2-hydroxyethyl)-2-(methyl(2-oxo-4-(o-tolyl)-2H-chromen-7-yl)amino)acetamide, 2-(methyl(2-oxo-4-(o-tolyl)-2H-chromen-7-yl)amino)-N-(2,2,2-trifluoroethyl)acetamide, N-(2-(dimethylamino)ethyl)-2-(methyl(2-oxo-4-(o-tolyl)-2H-chromen-7-yl)amino)acetamide

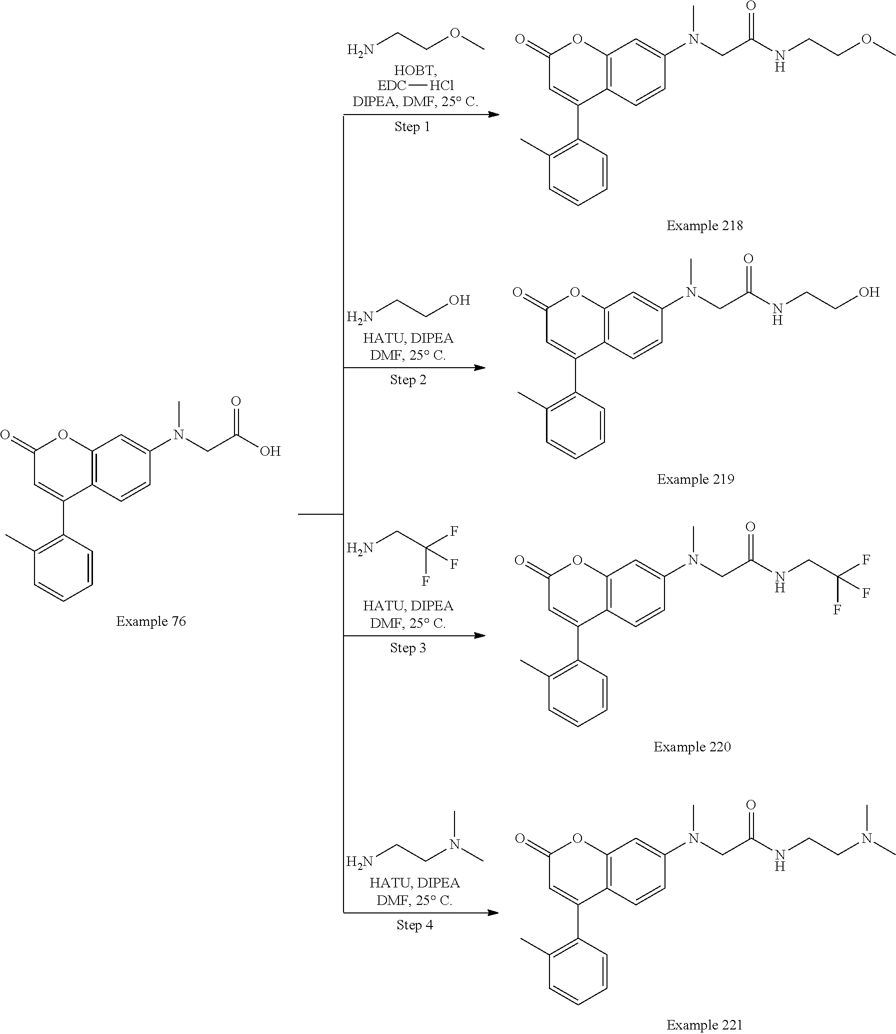

Synthesis of N-(2-methoxyethyl)-2-(methyl(2-oxo-4-(o-tolyl)-2H-chromen-7-yl)amino)acetamide, Example 218 [Step 1]: To a stirred solution of N-methyl-N-(2-oxo-4-(o-tolyl)-2H-chromen-7-yl)glycine (Example 76, 100 mg, 0.3 mmol) and 2-methoxyethan-1-amine (93 mg, 1.2 mmol) in DMF (2 mL) was successively added HOBT (65 mg, 0.5 mmol), EDC HCl (90 mg, 0.9 mmol), and DIPEA (0.2 mL, 1.2 mmol), and the reaction mixture was stirred at 25° C. After 16 h., the reaction mixture was quenched with cold water, and extracted with ethyl acetate (twice). The organic extracts were combined and washed with brine, dried over anhydrous Na₂SO₄, filtered, and concentrated under reduced pressure. The product purified by reverse phase prep-HPLC to afford N-(2-methoxyethyl)-2-(methyl(2-oxo-4-(o-tolyl)-2H-chromen-7-yl)amino)acetamide (Example 218, 16 mg). LCMS (ESI) Calcd. for C₂₂H₂₄N₂O₄: 380, found [M+H]⁺=381. ¹H NMR (400 MHz, DMSO-d₆) δ 8.08 (t, 1H), 7.41-7.37 (m, 2H), 7.35-7.31 (m, 1H), 7.21-7.19 (m, 1H), 6.72-6.70 (m, 1H), 6.60-6.58 (m, 2H), 5.92 (s, 1H), 4.05 (s, 2H), 3.31-3.29 (m, 2H), 3.25-3.22 (m, 5H), 3.06 (s, 3H), 2.11 (s, 3H).

Synthesis of N-(2-hydroxyethyl)-2-(methyl(2-oxo-4-(o-tolyl)-2H-chromen-7-yl)amino)acetamide, Example 219 [Step 2]: To a stirred solution of N-methyl-N-(2-oxo-4-(o-tolyl)-2H-chromen-7-yl)glycine (Example 76, 100 mg, 0.3 mmol) and 2-aminoethan-1-ol (45 mg, 0.5 mmol) in DMF (2 mL), was successively added HATU (175 mg, 0.5 mmol) and DIPEA (0.1 mL, 0.6 mmol), and the reaction mixture was stirred at 25° C. After 16 h., the reaction mixture was quenched with cold water, and extracted with ethyl acetate (twice). The organic extracts were combined and washed with brine, dried over anhydrous Na₂SO₄, filtered, and concentrated under reduced pressure. The product was purified by reverse phase prep-HPLC to afford N-(2-hydroxyethyl)-2-(methyl(2-oxo-4-(o-tolyl)-2H-chromen-7-yl)amino)acetamide (Example 219, 75 mg). LCMS (ESI) Calcd. for C₂₁H₂₂N₂O₄: 366, found [M+H]⁺=367. ¹H NMR (400 MHz, DMSO-d₆) δ 7.99 (t, 1H), 7.41-7.37 (m, 2H), 7.35-7.31 (m, 1H), 7.21-7.20 (m, 1H), 6.73-6.71 (m, 1H), 6.59-6.57 (m, 2H), 5.92 (s, 1H), 4.67 (t, 1H), 4.04 (s, 2H), 3.41-3.36 (m, 2H), 3.16-3.11 (m, 2H), 3.06 (s, 3H), 2.11 (s, 3H).

Synthesis of 2-(methyl(2-oxo-4-(o-tolyl)-2H-chromen-7-yl)amino)-N-(2,2,2-trifluoroethyl)acetamide, Example 220 [Step 3]: To a stirred solution of N-methyl-N-(2-oxo-4-(o-tolyl)-2H-chromen-7-yl)glycine (Example 76, 100 mg, 0.3 mmol) and 2,2,2-trifluoroethan-1-amine (63 mg, 0.5 mmol) in DMF (2 mL), was successively added HATU (175 mg, 0.5 mmol) and DIPEA (0.1 mL, 0.6 mmol), and the reaction mixture was stirred at 25° C. After 16 h., the reaction mixture was quenched with cold water, and extracted with ethyl acetate (twice). The organic extracts were combined and washed with brine, dried over anhydrous Na₂SO₄, filtered, and concentrated under reduced pressure. The product was purified by reverse phase prep-HPLC to afford 2-(methyl(2-oxo-4-(o-tolyl)-2H-chromen-7-yl)amino)-N-(2,2,2-trifluoroethyl)acetamide (Example 220, 51 mg). LCMS (ESI) Calcd. for C₂₁H₁₉F₃N₂O₃: 404, found [M+H]⁺=405. ¹H NMR (400 MHz, DMSO-d₆) δ 8.70 (t, 1H), 7.41-7.33 (m, 3H), 7.22-7.20 (m, 1H), 6.73-6.71 (m, 1H), 6.59-6.58 (m, 2H), 5.94 (s, 1H), 4.16 (s, 1H), 3.94-3.89 (m, 3H), 3.07 (s, 3H), 2.11 (s, 3H).

Synthesis of N-(2-(dimethylamino)ethyl)-2-(methyl(2-oxo-4-(o-tolyl)-2H-chromen-7-yl)amino)acetamide, Example 221 [Step 4]: To a stirred solution of N-methyl-N-(2-oxo-4-(o-tolyl)-2H-chromen-7-yl)glycine (Example 76, 100 mg, 0.3 mmol) and N′,N′-dimethylethane-1,2-diamine (40 mg, 0.5 mmol) in DMF (2 mL), was successively added HATU (176 mg, 0.5 mmol) and DIPEA (0.1 mL, 0.6 mmol), and the reaction mixture was stirred at 25° C. After 16 h., the reaction mixture was quenched with cold water, and extracted with ethyl acetate (twice). The organic extracts were combined and was washed with brine, dried over anhydrous Na₂SO₄, filtered, and concentrated under reduced pressure. The compound was purified by reverse phase prep-HPLC to afford N-(2-(dimethylamino)ethyl)-2-(methyl(2-oxo-4-(o-tolyl)-2H-chromen-7-yl)amino)acetamide (Example 221, 35 mg). LCMS (ESI) Calcd. for C₂₃H₂₇N₃O₃: 393, found [M+H]⁺=394. ¹H NMR (400 MHz, DMSO-d₆) δ 7.92 (t, 1H), 7.43-7.31 (m, 3H), 7.21-7.19 (m, 1H), 6.72-6.70 (m, 1H), 6.60-6.58 (m, 2H), 5.92 (s, 1H), 4.04 (s, 2H), 3.17-3.10 (m, 2H), 3.06 (s, 3H), 2.32 (t, 2H), 2.14-2.11 (m, 9H).

### Example 222: Synthesis of (R)-1-(methyl((2-oxo-4-(o-tolyl)-2H-chromen-7-yl)methyl)carbamoyl)piperidine-3-carboxylic acid

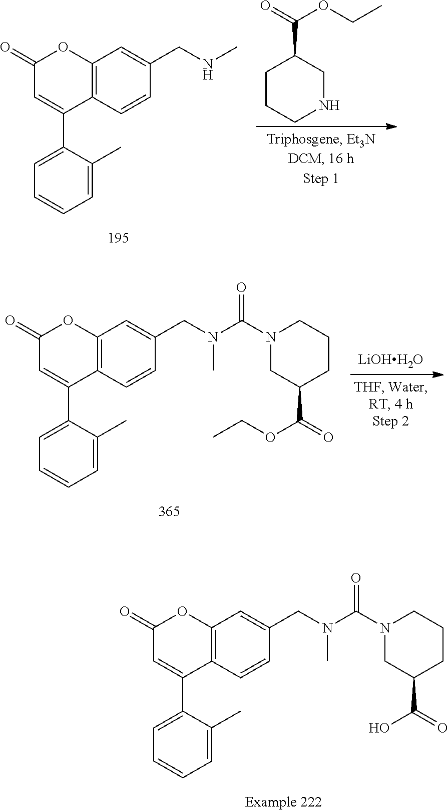

Synthesis of ethyl (R)-1-(methyl((2-oxo-4-(o-tolyl)-2H-chromen-7-yl)methyl)carbamoyl)piperidine-3-carboxylate, 365 [Step 1]: To a stirred solution of 7-((methylamino)methyl)-4-(o-tolyl)-2H-chromen-2-one (195, 180 mg, 0.6 mmol) in dichloromethane (3 mL) was added triethylamine (0.4 mL, 2.6 mmol) followed by triphosgene (285 mg, 1.0 mmol) at 0° C. and the reaction mixture was stirred at the same temperature for 15 min. Ethyl (R)-piperidine-3-carboxylate (110 mg, 0.7 mmol) was added to the reaction mixture and stirred at ambient temperature for 16 h. The reaction mixture was quenched with water and extracted with dichloromethane (thrice). The combined organic extracts was collected and washed with water, brine, dried over anhydrous Na₂SO₄, and concentrated under reduced pressure to afford ethyl (R)-1-(methyl((2-oxo-4-(o-tolyl)-2H-chromen-7-yl)methyl)carbamoyl)piperidine-3-carboxylate (365, 230 mg). LCMS (ESI) Calcd. for C₂₇H₃₀N₂O₅: 462, found [M+H]⁺=463.

Synthesis of (R)-1-(methyl((2-oxo-4-(o-tolyl)-2H-chromen-7-yl)methyl)carbamoyl)piperidine-3-carboxylic acid, Example 222 [Step 2]: To a stirred solution of ethyl (R)-1-(methyl((2-oxo-4-(o-tolyl)-2H-chromen-7-yl)methyl)carbamoyl)piperidine-3-carboxylate (365, 150 mg, 0.3 mmol) in THF (4 mL) and water (1 mL), was added LiOH H₂O (40 mg, 1.0 mmol) and the reaction mixture was stirred at ambient temperature for 4 h. The reaction mixture was concentrated under reduced pressure, diluted with water and acidified with 1N HCl. The reaction mixture was extracted with ethyl acetate and washed with water, brine, dried over anhydrous Na₂SO₄, and concentrated under reduced pressure. The product was purified by reverse phase prep-HPLC and lyophilized to afford (R)-1-(methyl((2-oxo-4-(o-tolyl)-2H-chromen-7-yl)methyl)carbamoyl)piperidine-3-carboxylic acid (Example 222, 36 mg). LCMS (ESI) Calcd. for C₂₅H₂₆N₂O₅: 434, found [M+H]⁺=435. ¹H NMR (400 MHz, DMSO-d₆) δ 7.45-7.39 (m, 2H), 7.37-7.34 (m, 2H), 7.25 (d, 1H), 7.16 (d, 1H), 6.95 (d, 1H), 6.35 (s, 1H), 4.46-4.34 (m, 2H), 3.60 (d, 1H), 3.40 (d, 1H), 2.88-2.76 (m, 2H), 2.71 (s, 3H), 2.39-2.34 (m, 1H), 2.11 (s, 3H), 1.91-1.89 (m, 1H), 1.63-1.60 (m, 1H), 1.54-1.40 (m, 2H).

### Example 223: Synthesis of N-methyl-N-(2-oxo-4-(o-tolyl)-2H-chromen-7-yl)glycylglycine

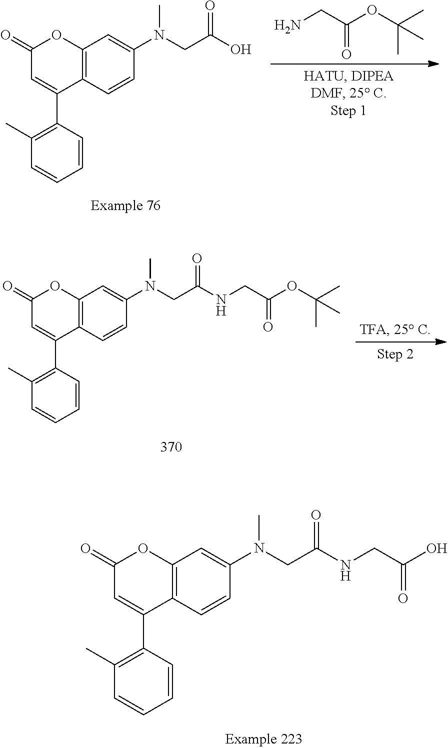

Synthesis of tert-butyl N-methyl-N-(2-oxo-4-(o-tolyl)-2H-chromen-7-yl)glycylglycinate, 370 [Step 1]: To a solution of N-methyl-N-(2-oxo-4-(o-tolyl)-2H-chromen-7-yl)glycine (Example 76, 150 mg, 0.5 mmol) and tert-butyl glycinate (2, 91 mg, 0.7 mmol) in DMF (5 mL), was successively added HATU (265 mg, 0.7 mmol) and DIPEA (0.2 mL, 0.9 mmol), and the reaction mixture was stirred at 25° C. After 16 h., the reaction mixture was quenched with cold water, and extracted with ethyl acetate (twice). The organic extracts were combined and washed with brine, dried over anhydrous Na₂SO₄, filtered, and concentrated under reduced pressure to afford tert-butyl N-methyl-N-(2-oxo-4-(o-tolyl)-2H-chromen-7-yl)glycylglycinate (370, 150 mg). LCMS (ESI) Calcd. for C₂₅H₂₈N₂O₅:436, found [M+H]⁺=437.

Synthesis of N-methyl-N-(2-oxo-4-(o-tolyl)-2H-chromen-7-yl)glycylglycine, Example 223 [Step 2]: A solution of tert-butyl N-methyl-N-(2-oxo-4-(o-tolyl)-2H-chromen-7-yl)glycylglycinate (370, 150 mg, 0.3 mmol) in TFA (1.0 mL, 13.1 mmol) was stirred at 25° C. After 16 h., the reaction mixture was concentrated under reduced pressure, and the product was purified by reverse phase prep-HPLC to afford N-methyl-N-(2-oxo-4-(o-tolyl)-2H-chromen-7-yl)glycylglycine (Example 223, 24 mg). LCMS (ESI) Calcd. for C₂₁H₂₀N₂O₅: 380, found [M+H]⁺=381. ¹H NMR (400 MHz, DMSO-d₆) δ 12.56 (br s, 1H), 8.29-8.27 (m, 1H), 7.43-7.37 (m, 2H), 7.35-7.31 (m, 1H), 7.21-7.20 (m, 1H), 6.72-6.70 (m, 1H), 6.62-6.60 (m, 2H), 5.93 (s, 1H), 4.10 (s, 2H), 3.75-3.73 (m, 2H), 3.07 (s, 3H), 2.11 (s, 3H).

### Example 224-225: Synthesis of ethyl (1R,2S)-2-(2-oxo-4-(o-tolyl)-2H-chromen-7-yl)cyclopropane-1-carboxylate and ethyl (1S,2S)-2-(2-oxo-4-(o-tolyl)-2H-chromen-7-yl)cyclopropane-1-carboxylate

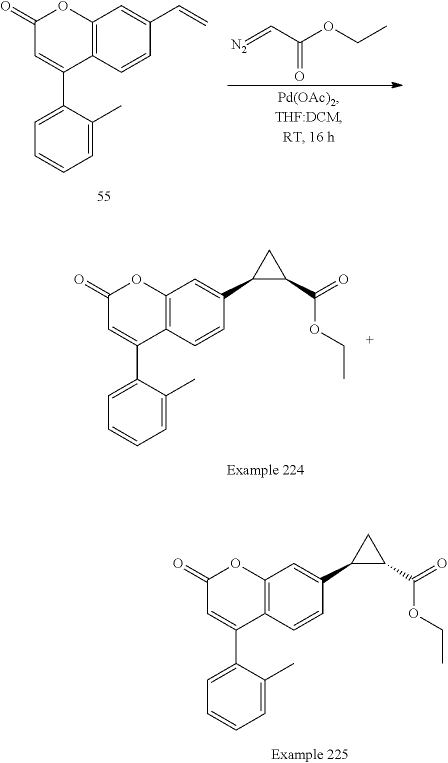

Synthesis of ethyl (1R,2S)-2-(2-oxo-4-(o-tolyl)-2H-chromen-7-yl)cyclopropane-1-carboxylate, Example 224, and ethyl (1S,2S)-2-(2-oxo-4-(o-tolyl)-2H-chromen-7-yl)cyclopropane-1-carboxylate, Example 225: To a solution of 4-(o-tolyl)-7-vinyl-2H-chromen-2-one (55, 500 mg, 1.9 mmol) in THF (8 mL) was added palladium acetate (40 mg, 0.2 mmol) followed by a solution of ethyl 2-diazoacetate (435 mg, 3.8 mmol) in DCM (1.2 mL) at ambient temperature. The reaction mixture was stirred at ambient temperature for 16 h., and then filtered through a celite bed and washed with ethyl acetate. The filtrate was concentrated under reduced pressure, and the product was purified by RP prep-HPLC to obtain Peak 1 as ethyl (1R,2S)-2-(2-oxo-4-(o-tolyl)-2H-chromen-7-yl)cyclopropane-1-carboxylate (Example 224, 130 mg), and Peak 2 as ethyl (1S,2S)-2-(2-oxo-4-(o-tolyl)-2H-chromen-7-yl)cyclopropane-1-carboxylate (Example 225, 130 mg).

Example 224: Ethyl (1R,2S)-2-(2-oxo-4-(o-tolyl)-2H-chromen-7-yl)cyclopropane-1-carboxylate, Peak 1: LCMS (ESI) Calcd. for C₂₂H₂₀O₄: 348, found [M+H]⁺=349. ¹H NMR (400 MHz, DMSO-d₆) δ 7.44-7.37 (m, 2H), 7.35 (d, 2H), 7.25 (d, 1H), 7.18 (d, 1H), 6.87 (d, 1H), 6.32 (s, 1H), 3.84-3.80 (m, 2H), 2.74 (d, 1H), 2.19 (d, 1H), 2.09 (d, 3H), 1.66 (d, 1H), 1.43-1.41 (m, 1H), 0.93-0.87 (m, 3H). 0588 Example 225: Ethyl (1S,2S)-2-(2-oxo-4-(o-tolyl)-2H-chromen-7-yl)cyclopropane-1-carboxylate, Peak 2: LCMS (ESI) Calcd. for C₂₂H₂₀O₄: 348, found [M+H]⁺=349. ¹H NMR (400 MHz, DMSO-d₆) δ 7.42-7.37 (m, 2H), 7.35 (d, 2H), 7.25 (d, 1H), 7.13-7.10 (m, 1H), 6.85 (d, 1H), 6.31 (s, 1H), 4.12-4.07 (m, 2H), 2.56-2.53 (m, 1H), 2.09 (d, 4H), 1.54-1.47 (m, 2H), 1.21 (t, 3H).

### Example 226-227: Synthesis of (cis)-2-(2-oxo-4-(o-tolyl)-2H-chromen-7-yl)cyclopropane-1-carboxylic acid, (trans)-2-(2-oxo-4-(o-tolyl)-2H-chromen-7-yl)cyclopropane-1-carboxylic acid

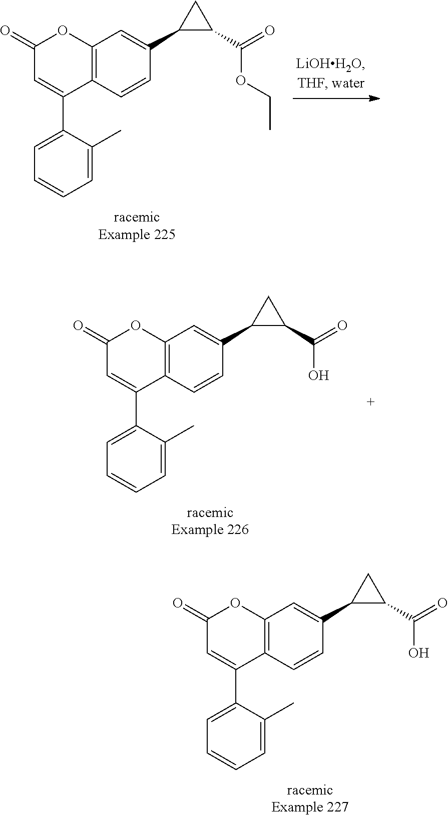 [SMILES: CCOC(=O)[C@H]1C[C@@H]1c1ccc2c(-c3ccccc3C)cc(=O)oc2c1 ; Cc1ccccc1-c1cc(=O)oc2cc([C@H]3C[C@H]3C(=O)O)ccc12 ; Cc1ccccc1-c1cc(=O)oc2cc([C@H]3C[C@@H]3C(=O)O)ccc12]

Synthesis of (Cis)- and (trans)-2-(2-oxo-4-(o-tolyl)-2H-chromen-7-yl)cyclopropane-1-carboxylic acid, Example 226 and Example 227: To the stirred solution of ethyl (trans)-2-(2-oxo-4-(o-tolyl)-2H-chromen-7-yl)cyclopropane-1-carboxylate (Example 225, 100 mg, 0.3 mmol) in THF:water (5:1 mL) mixture, was added LiOH H₂O (25 mg, 0.6 mmol) in an ice-bath and the reaction mixture was stirred at ambient temperature for 16 h. The reaction mixture was concentrated under reduced pressure and the residue was acidified with 1N HCl to pH=3-4. The mixture was extracted with EtOAc (3×30 mL) and the organic phases were combined and washed with brine, dried over anhydrous Na₂SO₄, filtered, and concentrated under reduced pressure. The mixture of products was purified by RP prep-HPLC purification and lyophilized to yield Peak 1 as (cis)-2-(2-oxo-4-(o-tolyl)-2H-chromen-7-yl)cyclopropane-1-carboxylic acid (Example 226, 25 mg) and Peak 2 as (trans)-2-(2-oxo-4-(o-tolyl)-2H-chromen-7-yl)cyclopropane-1-carboxylic acid (Example 227, 35 mg, 0.109 mmol).

Example 226: (Cis)-2-(2-oxo-4-(o-tolyl)-2H-chromen-7-yl)cyclopropane-1-carboxylic acid, Peak 1: LCMS (ESI) Calcd. for C₂₀H₁₆O₄: 320, found [M+H]⁺=321. ¹H NMR (400 MHz, DMSO-d₆) δ 10.86 (br s, 1H), 7.45-7.31 (m, 4H), 7.24-7.17 (m, 2H), 6.85 (d, 1H), 6.31 (s, 1H), 2.62-2.54 (m, 1H), 2.10-2.05 (m, 4H), 1.59-1.53 (m, 1H), 1.35-1.30 (m, 1H).

Example 227: (Trans)-2-(2-oxo-4-(o-tolyl)-2H-chromen-7-yl)cyclopropane-1-carboxylic acid, Peak 2: LCMS (ESI) Calcd. for C₂₀H₁₆O₄: 320, found [M+H]⁺=321. ¹H NMR (400 MHz, DMSO-d₆) δ 7.43-7.23 (m, 5H), 7.08 (d, 1H), 6.84 (d, 1H), 6.30 (s, 1H), 2.45-2.41 (m, 1H), 2.10 (s, 3H), 1.89 (br s, 1H), 1.46-1.34 (m, 2H).

### Example 228: Synthesis of (S)-3-(methyl(2-oxo-4-(o-tolyl)-2H-chromen-7-yl)amino)butanoic acid

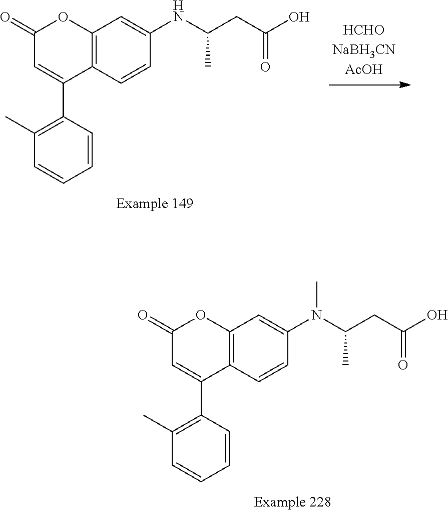 [SMILES: Cc1ccccc1-c1cc(=O)oc2cc(N[C@@H](C)CC(=O)O)ccc12 ; Cc1ccccc1-c1cc(=O)oc2cc(N(C)[C@@H](C)CC(=O)O)ccc12]

Synthesis of (S)-3-(methyl(2-oxo-4-(o-tolyl)-2H-chromen-7-yl)amino)butanoic acid, Example 228: To a stirred solution of (S)-3-((2-oxo-4-(o-tolyl)-2H-chromen-7-yl)amino)butanoic acid (Example 149, 150 mg, 0.4 mmol) and formaldehyde (0.1 mL, 4.4 mmol, 37% in aq. soln) in acetic acid (2 mL) was added sodium cyanoborohydride (85 mg, 1.3 mmol) at 0° C. The reaction mixture was stirred at ambient temperature for 30 min., and was concentrated under reduced pressure. The product was purified by RP prep-HPLC to afford(S)-3-(methyl(2-oxo-4-(o-tolyl)-2H-chromen-7-yl)amino)butanoic acid (Example 228, 60 mg). LCMS (ESI) Calcd. for C₂₁H₂₁NO₄: 351, found [M+H]⁺=352. ¹H NMR (400 MHz, methanol-d₄) δ 7.40-7.27 (m, 3H), 7.19-7.17 (m, 1H), 6.85-6.83 (m, 1H), 6.81-6.76 (m, 2H), 5.90 (s, 1H), 4.62-4.58 (m, 1H), 2.87 (s, 3H), 2.64-2.57 (m, 1H), 2.54-2.48 (m, 1H), 2.15 (s, 3H), 1.26-1.24 (m, 3H).

### Example 229: Synthesis of 4-(methyl((2-oxo-4-(o-tolyl)-2H-chromen-7-yl)methyl)carbamoyl)cyclohexane-1-carboxylic acid

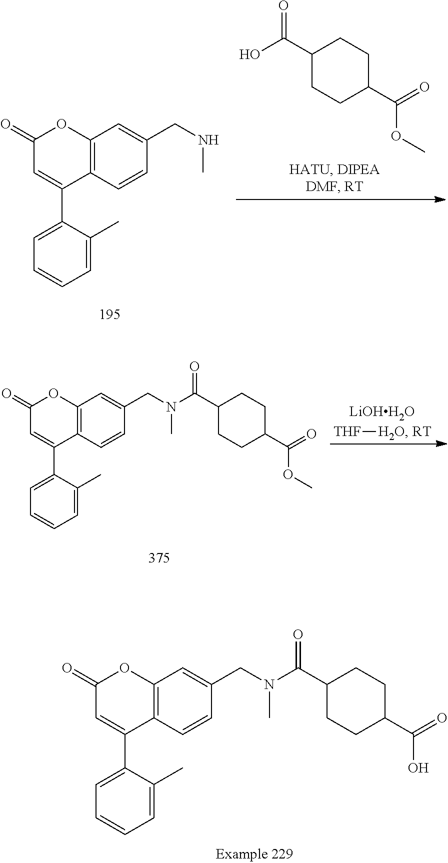

Synthesis of methyl 4-(methyl((2-oxo-4-(o-tolyl)-2H-chromen-7-yl)methyl)carbamoyl)cyclohexane-1-carboxylate, 375 [Step 1]: To a stirred solution of 7-((methylamino)methyl)-4-(o-tolyl)-2H-chromen-2-one (195, 130 mg, 0.5 mmol) and 4-(methoxycarbonyl)cyclohexane-1-carboxylic acid (105 mg, 0.6 mmol) in DMF (3 mL) was added HATU (195 mg, 0.5 mmol) followed by DIPEA (0.1 mL, 0.7 mmol), and the reaction mixture was stirred at ambient temperature. After 16 h., the reaction mixture was quenched with ice-cold water, and extracted with EtOAc (twice). The combined organic phase was washed with cold brine, dried over anhydrous Na₂SO₄, filtered, and concentrated under reduced pressure to afford methyl 4-(methyl((2-oxo-4-(o-tolyl)-2H-chromen-7-yl)methyl)carbamoyl)cyclohexane-1-carboxylate (375, 200 mg). LCMS (ESI) Calcd. for C₂₇H₂₉NO₅: 447, found [M+H]⁺=448.

Synthesis of 4-(methyl((2-oxo-4-(o-tolyl)-2H-chromen-7-yl)methyl)carbamoyl)cyclohexane-1-carboxylic acid, Example 229 [Step 2]: To a solution of 4-(methyl((2-oxo-4-(o-tolyl)-2H-chromen-7-yl)methyl)carbamoyl)cyclohexane-1-carboxylate (375, 200 mg, 0.4 mmol) in THF:water (4:1 mL) mixture was added lithium hydroxide monohydrate (190 mg, 4.5 mmol), and the reaction mixture was stirred at ambient temperature for 12 h. The reaction mixture was diluted with ethyl acetate and water and the water layer was collected and acidified with 2N HCl to pH 2-3. The mixture was extracted using ethyl acetate and washed with water and brine. The organic phase was dried over anhydrous Na₂SO₄. and concentrated under reduced pressure. The product was purified by RP prep-HPLC and lyophilized to afford 4-(methyl((2-oxo-4-(o-tolyl)-2H-chromen-7-yl)methyl)carbamoyl)cyclohexane-1-carboxylic acid (Example 229, 55 mg). LCMS (ESI) Calcd. for C₂₆H₂₇NO₅: 433, found [M+H]⁺=434. ¹H NMR (400 MHz DMSO-d₆) (at 100° C.) δ 7.46-7.33 (m, 3H), 7.26-7.24 (m, 2H) 7.11-7.09 (m, 1H), 6.99-6.96 (m, 1H), 6.29 (s, 1H), 4.64 (s, 2H), 2.66-2.53 (m, 1H) 2.23-2.17 (m, 1H), 2.14 (s, 3H), 1.97-1.94 (m, 2H), 1.79-1.77 (m, 2H), 1.52-1.36 (m, 4H).

### Example 230: Synthesis of (R)-3-(methyl(2-oxo-4-(o-tolyl)-2H-chromen-7-yl)amino)butanoic acid

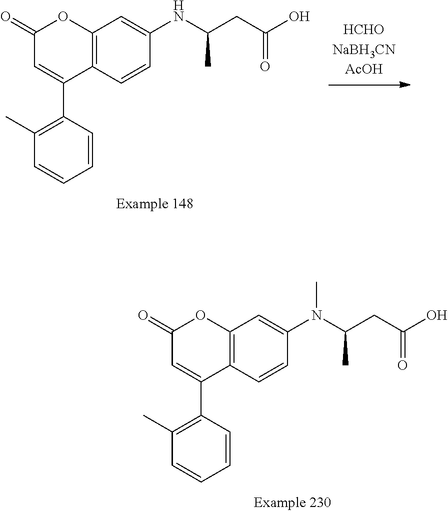 [SMILES: Cc1ccccc1-c1cc(=O)oc2cc(N[C@H](C)CC(=O)O)ccc12 ; Cc1ccccc1-c1cc(=O)oc2cc(N(C)[C@H](C)CC(=O)O)ccc12]

Synthesis of (R)-3-(methyl(2-oxo-4-(o-tolyl)-2H-chromen-7-yl)amino)butanoic acid, Example 230: To a stirred solution of (R)-3-((2-oxo-4-(o-tolyl)-2H-chromen-7-yl)amino)butanoic acid (Example 148, 150 mg, 0.4 mmol) and formaldehyde (0.1 mL, 4.4 mmol, 37% in aq. soln) in acetic acid (2 mL) was added sodium cyanoborohydride (85 mg, 1.3 mmol) at 0° C. The reaction mixture was stirred at ambient temperature for 30 min., then concentrated under reduced pressure. The product was purified by RP Prep-HPLC to afford (R)-3-(methyl(2-oxo-4-(o-tolyl)-2H-chromen-7-yl)amino)butanoic acid (Example 230, 21 mg). LCMS (ESI) Calcd. for C₂₁H₂₁NO₄: 351, found [M+H]⁺=352. ¹H NMR (400 MHz, methanol-d₄) δ 7.38-7.29 (m, 3H), 7.19-7.17 (m, 1H), 6.86-6.83 (m, 1H), 6.80-6.76 (m, 2H), 5.90 (s, 1H), 4.62-4.57 (m, 1H), 2.87 (s, 3H) 2.67-2.61 (m, 1H), 2.56-2.51 (m, 1H), 2.15 (s, 3H), 1.27-1.25 (m, 3H).

### Example 231: Synthesis of (cis)-N-methyl-2-(2-oxo-4-(o-tolyl)-2H-chromen-7-yl)cyclopropane-1-carboxamide

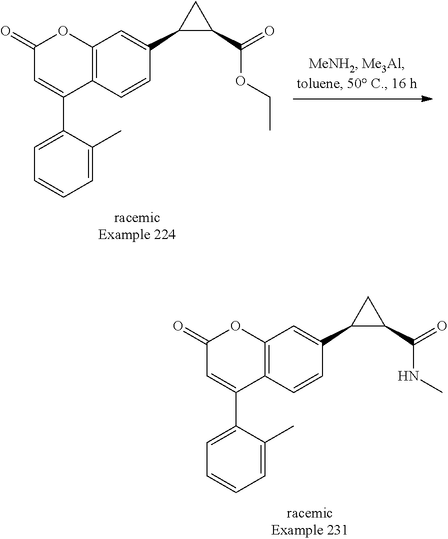 [SMILES: CCOC(=O)[C@@H]1C[C@@H]1c1ccc2c(-c3ccccc3C)cc(=O)oc2c1 ; CNC(=O)[C@@H]1C[C@@H]1c1ccc2c(-c3ccccc3C)cc(=O)oc2c1]

Synthesis of (cis)-N-methyl-2-(2-oxo-4-(o-tolyl)-2H-chromen-7-yl)cyclopropane-1-carboxamide, Example 231: A stirred solution of ethyl (cis)-2-(2-oxo-4-(o-tolyl)-2H-chromen-7-yl)cyclopropane-1-carboxylate (Example 224, 100 mg, 0.3 mmol) in toluene (2 mL) was added methyl amine in THF (635 mg, 1.4 mmol, 2 M). The mixture was cooled in ice and trimethylaluminum solution in toluene (40 mg, 0.6 mmol, 2 M) was added dropwise to the mixture. The reaction mixture was heated at 50° C. and stirred for 16 h. The reaction mixture was diluted with ethyl acetate and washed with aqueous NH₄Cl solution, water, and brine. The organic phase was dried over anhydrous Na₂SO₄, filtered, and concentrated under reduced pressure. The product was purified by RP prep-HPLC to yield (cis)-N-methyl-2-(2-oxo-4-(o-tolyl)-2H-chromen-7-yl)cyclopropane-1-carboxamide (Example 231, 26 mg). LCMS (ESI) Calcd. for C₂₁H₂₀NO₃: 334, found [M+H]⁺=334. ¹H NMR (400 MHz, DMSO-d₆) δ 7.98 (t, 1H), 7.46-7.33 (m, 3H), 7.28-7.23 (m, 2H), 7.15 (d, 1H), 6.82 (d, 1H), 6.30 (s, 1H), 2.47-2.42 (m, 4H), 2.11 (s, 3H), 2.08-2.02 (m, 1H), 1.57-1.52 (m, 1H), 1.29-1.24 (m, 1H).

### Example 232: Synthesis of (trans)-N-methyl-2-(2-oxo-4-(o-tolyl)-2H-chromen-7-yl)cyclopropane-1-carboxamide

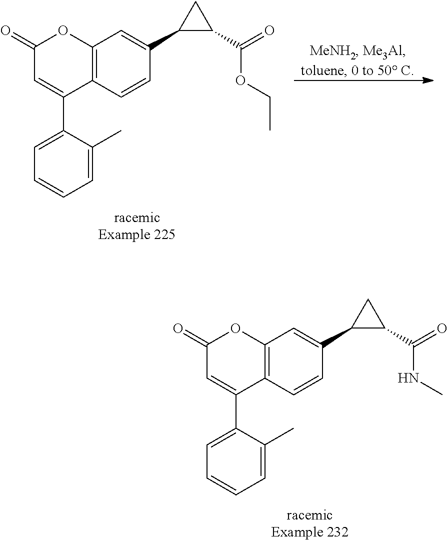 [SMILES: CCOC(=O)[C@H]1C[C@@H]1c1ccc2c(-c3ccccc3C)cc(=O)oc2c1 ; CNC(=O)[C@H]1C[C@@H]1c1ccc2c(-c3ccccc3C)cc(=O)oc2c1]

Synthesis of (trans)-N-methyl-2-(2-oxo-4-(o-tolyl)-2H-chromen-7-yl)cyclopropane-1-carboxamide, Example 232: A mixture of ethyl (trans)-2-(2-oxo-4-(o-tolyl)-2H-chromen-7-yl)cyclopropane-1-carboxylate (Example 225, 100 mg, 0.3 mmol) in toluene (2 mL) was stirred and cooled to 0° C. and methyl amine in THF (0.7 mL, 1.4 mmol, 2 M) was added dropwise followed by trimethyl aluminum solution in toluene (0.6 mL, 1.1 mmol, 2 M). The reaction mixture was stirred at 50° C. for 12 h., and quenched with ice-cold water and extracted with ethyl acetate. The organic layer was washed with brine, dried over anhydrous Na₂SO₄, and concentrated under reduced pressure. The product was purified by RP prep-HPLC and lyophilized to afford (trans)-N-methyl-2-(2-oxo-4-(o-tolyl)-2H-chromen-7-yl)cyclopropane-1-carboxamide (Example 232, 30 mg). LCMS (ESI) Calcd. for C₂₁H₂₀O₃: 334, found [M+H]⁺: 334. ¹H NMR (400 MHz, DMSO-d₆) δ 8.07 (d, 1H), 7.45-7.33 (m, 3H), 7.29 (d, 1H), 7.25 (d, 1H), 7.07-7.03 (m, 1H), 6.85 (d, 1H), 6.30 (s, 1H), 2.61 (d, 3H), 2.35-2.32 (m, 1H), 2.09 (s, 3H), 1.93-1.90 (m, 1H), 1.44-1.39 (m, 1H), 1.35-1.32 (m, 1H).

### Example 233: Synthesis of 2-(2-oxo-4-(o-tolyl)-2H-chromen-7-yl) acetic acid

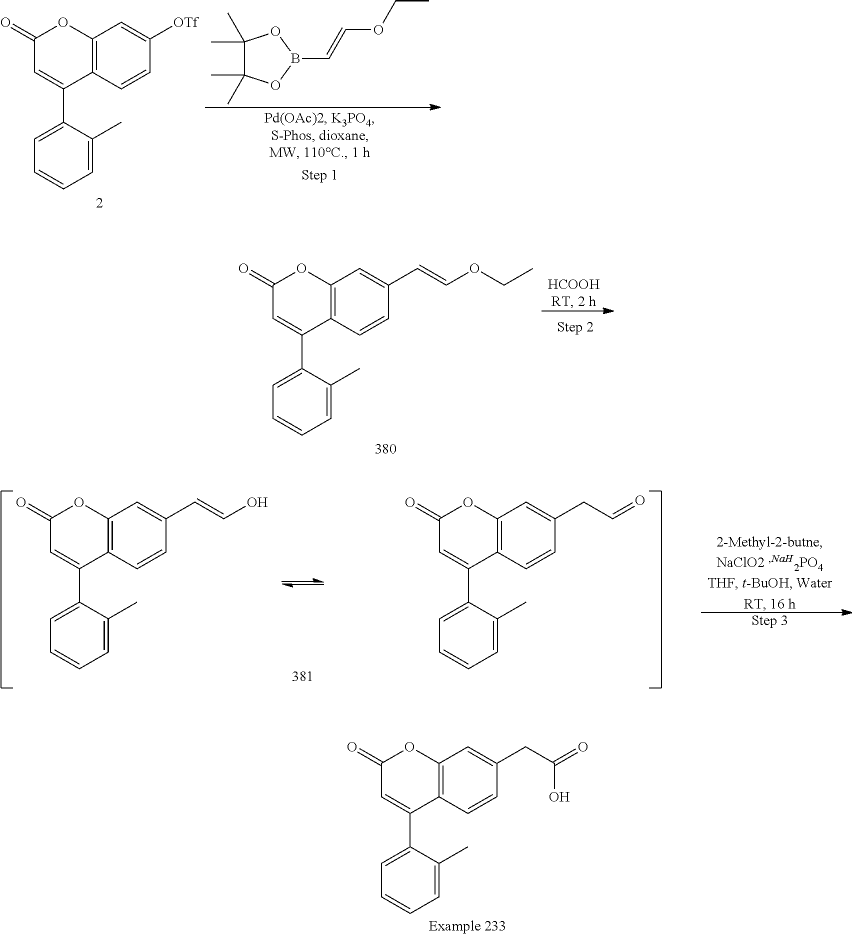

Synthesis of (E)-7-(2-ethoxyvinyl)-4-(o-tolyl)-2H-chromen-2-one, 380 [Step 1]: In a microwave vial was added a mixture of 2-oxo-4-(o-tolyl)-2H-chromen-7-yl trifluoromethanesulfonate (2, 300 mg, 0.8 mmol) in 1,4-dioxane (2 mL) followed by the addition of (E)-2-(2-ethoxyvinyl)-4,4,5,5-tetramethyl-1,3,2-dioxaborolane (0.3 mL, 1.5 mmol) and the reaction mixture was purged with argon gas for 5 min. To the reaction mixture was added K₃PO₄ (130 mg, 1.5 mmol), S-Phos (45 mg, 0.2 mmol) and Pd(OAc)₂ (20 mg, 0.1 mmol), and the reaction mixture was irradiated in microwave for at 110° C. for 1 h. The reaction mixture was filtered through a celite bed and the filtrate was concentrated under reduced pressure. The product was purified by flash column chromatography to afford (E)-7-(2-ethoxyvinyl)-4-(o-tolyl)-2H-chromen-2-one (380, 120 mg). LCMS (ESI): Calcd. for C₂₀H₁₈O₃: 306, found [M+H]⁺=307. ¹H NMR (400 MHz, DMSO-d₆) δ 7.49 (d, 1H), 7.41-7.38 (m, 3H), 7.36-7.33 (m, 1H), 7.24 (d, 1H), 7.20 (d, 1H), 6.80 (d, 1H), 6.22 (s, 1H), 5.91 (d, 1H), 3.94 (q, 2H), 2.11 (s, 3H), 1.26 (t, 3H).

Synthesis of 2-(2-oxo-4-(o-tolyl)-2H-chromen-7-yl) acetaldehyde, 381 [Step 2]: A round bottom flask was charged with (E)-7-(2-ethoxyvinyl)-4-(o-tolyl)-2H-chromen-2-one (380, 160 mg, 0.5 mmol) followed by the dropwise addition of formic acid (0.5 mL, 13.1 mmol) at 0° C., and the reaction mixture was stirred at ambient temperature for 2 h. The reaction mixture was quenched with water and extracted with DCM. The combined organic layer was washed with water, brine, dried over anhydrous Na₂SO₄, and concentrated under reduced pressure to afford 2-(2-oxo-4-(o-tolyl)-2H-chromen-7-yl) acetaldehyde (381, 135 mg). LCMS (ESI) Calcd. for C₁₈H₁₄O₃: 278, found [M+H]⁺=279.

Synthesis of 2-(2-oxo-4-(o-tolyl)-2H-chromen-7-yl) acetic acid, Example 233 [Step 3]: To a stirred solution of 2-(2-oxo-4-(o-tolyl)-2H-chromen-7-yl) acetaldehyde (381, 180 mg, 0.7 mmol), 2-methyl-2-butene (0.4 mL, 3.2 mmol) in a mixture of THF (4 mL) and 1-BuOH (2 mL) was added an aqueous solution of NaClO₂ (290 mg, 3.2 mmol) and NaH₂PO₄ (390 mg, 3.2 mmol) dropwise at 0° C. The reaction mixture was stirred vigorously at ambient temperature for 16 h., and then diluted with EtOAc and washed with brine. The organic layer was collected, dried over anhydrous Na₂SO₄, and concentrated under reduced pressure. The product was purified by RP prep-HPLC and lyophilized to afford 2-(2-oxo-4-(o-tolyl)-2H-chromen-7-yl) acetic acid (Example 233, 63 mg). LCMS (ESI) Calcd. for C₁₈H₁₄O₄: 294, found [M+H]⁺=295. ¹H NMR (400 MHz, DMSO-d₆) δ 12.60 (br s, 1H), 7.46-7.41 (m, 3H), 7.37-7.34 (m, 1H), 7.25 (d, 1H), 7.18 (d, 1H), 6.92 (d, 1H), 6.34 (s, 1H), 3.71 (s, 2H), 2.12 (s, 3H).

### Example 234: Synthesis of (cis)-N,N-dimethyl-2-(2-oxo-4-(o-tolyl)-2H-chromen-7-yl)cyclopropane-1-carboxamide

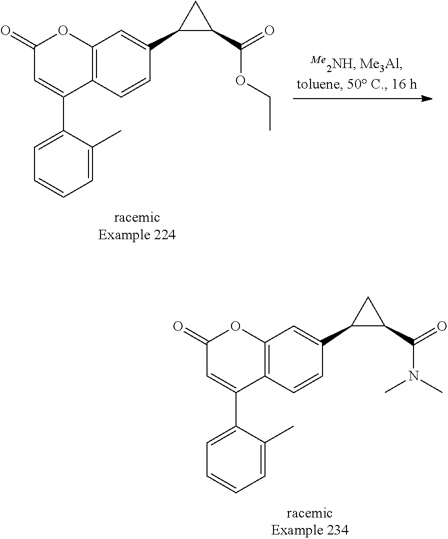 [SMILES: CCOC(=O)[C@@H]1C[C@@H]1c1ccc2c(-c3ccccc3C)cc(=O)oc2c1 ; Cc1ccccc1-c1cc(=O)oc2cc([C@H]3C[C@H]3C(=O)N(C)C)ccc12]

Synthesis of (Cis)-N,N-dimethyl-2-(2-oxo-4-(o-tolyl)-2H-chromen-7-yl)cyclopropane-1-carboxamide, Example 234: A stirred solution of ethyl (cis)-2-(2-oxo-4-(o-tolyl)-2H-chromen-7-yl)cyclopropane-1-carboxylate (Example 224, 50 mg, 0.1 mmol) in toluene (1 mL) was added dimethylamine in THF (0.35 mL, 0.7 mmol, 2 M), and the mixture was cooled in ice. To the mixture was added dropwise trimethylaluminum solution in toluene (2 mg, 0.3 mmol, 2 M). The reaction mixture was heated at 50° C. for 16 h., diluted with ethyl acetate, washed with aqueous NH₄Cl solution, water, and brine. The organic phase was dried over anhydrous Na₂SO₄, filtered, and concentrated under reduced pressure. The product was purified by RP prep-HPLC and lyophilized to afford (cis)-N,N-dimethyl-2-(2-oxo-4-(o-tolyl)-2H-chromen-7-yl)cyclopropane-1-carboxamide (Example 234, 32 mg). LCMS (ESI) Calcd. for C₂₂H₂₁NO₃: 347, found [M+H]⁺=349. ¹H NMR (400 MHz, DMSO-d₆) δ 7.43-7.33 (m, 3H), 7.26-7.19 (m, 2H), 7.10-7.05 (m, 1H), 6.83 (d, 1H), 6.30 (s, 1H), 2.99 (d, 3H), 2.61 (br s, 4H), 2.46-2.42 (m, 1H), 2.09 (br s, 3H), 1.63-1.58 (m, 1H), 1.31-1.29 (m, 1H).

### Example 235: Synthesis of (cis)-2-(2-oxo-4-(o-tolyl)-2H-chromen-7-yl)cyclopropane-1-carboxamide

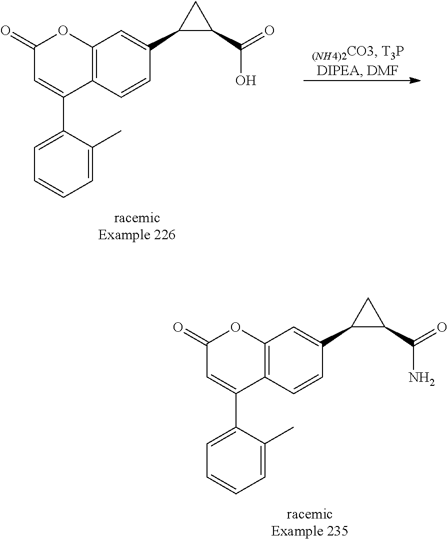 [SMILES: Cc1ccccc1-c1cc(=O)oc2cc([C@H]3C[C@H]3C(=O)O)ccc12 ; Cc1ccccc1-c1cc(=O)oc2cc([C@H]3C[C@H]3C(N)=O)ccc12]

Synthesis of (Cis)-2-(2-oxo-4-(o-tolyl)-2H-chromen-7-yl)cyclopropane-1-carboxamide, Example 235: A stirred solution of (cis)-2-(2-oxo-4-(o-tolyl)-2H-chromen-7-yl)cyclopropane-1-carboxylic acid (Example 226, 100 mg, 0.3 mmol) and ammonium carbonate (300 mg, 3.1 mmol) in DMF (2 mL) was cooled in ice. To the mixture was added dropwise T₃P (0.2 mL, 0.6 mmol, 50% in EtOAc) and DIPEA (0.3 mL, 1.6 mmol), and the reaction mixture was warmed to ambient temperature and stirred for 16 h. The reaction mixture was diluted with ethyl acetate and washed with aq. NH₄Cl solution, water, and brine. The organic phase was dried over anhydrous Na₂SO₄, filtered, and concentrated under reduced pressure. The product was purified by RP prep-HPLC to afford (cis)-2-(2-oxo-4-(o-tolyl)-2H-chromen-7-yl)cyclopropane-1-carboxamide (Example 235, 50 mg). LCMS (ESI) Calcd. for C₂₀H₁₇NO₃: 319, found [M+H]⁺=320. ¹H NMR (400 MHz, DMSO-d₆) δ 7.51 (br s, 1H), 7.45-7.33 (m, 3H), 7.28-7.23 (m, 2H), 7.17-7.14 (m, 1H), 6.83 (d, 1H), 6.70 (d, 1H), 6.30 (s, 1H), 2.45 (br s, 1H), 2.11-2.04 (m, 4H), 1.54-1.52 (m, 1H), 1.26-1.21 (m, 1H).

### Example 236: Synthesis of (trans)-N,N-dimethyl-2-(2-oxo-4-(o-tolyl)-2H-chromen-7-yl)cyclopropane-1-carboxamide

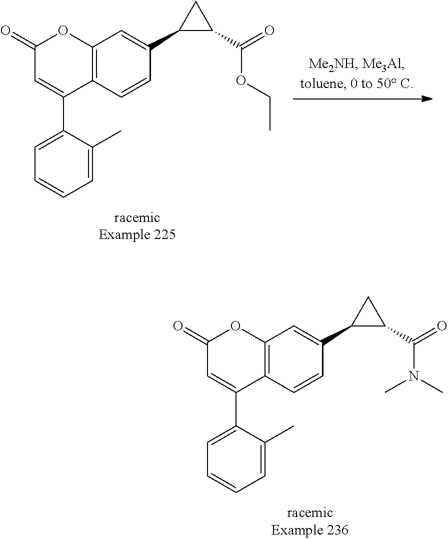 [SMILES: CCOC(=O)[C@H]1C[C@@H]1c1ccc2c(-c3ccccc3C)cc(=O)oc2c1 ; Cc1ccccc1-c1cc(=O)oc2cc([C@H]3C[C@@H]3C(=O)N(C)C)ccc12]

Synthesis of (trans)-N,N-dimethyl-2-(2-oxo-4-(o-tolyl)-2H-chromen-7-yl)cyclopropane-1-carboxamide, Example 236: To a sealed vial was added ethyl (trans)-2-(2-oxo-4-(o-tolyl)-2H-chromen-7-yl)cyclopropane-1-carboxylate (Example 225, 100 mg, 0.3 mmol) in toluene (2 mL), and the mixture was cooled to 0° C. followed by the dropwise addition of dimethylamine in THF (0.7 mL, 1.4 mmol, 2 M) and trimethylaluminum solution in toluene (0.3 mL, 0.6 mmol, 2 M). The reaction mixture was heated at 50° C. for 12 h., and then quenched with ice water and extracted with ethyl acetate, washed with brine, dried over anhydrous Na₂SO₄, and concentrated under reduced pressure. The product was purified by RP prep-HPLC purification and lyophilized to afford (trans)-N,N-dimethyl-2-(2-oxo-4-(o-tolyl)-2H-chromen-7-yl)cyclopropane-1-carboxamide (Example 236, 40 mg). LCMS (ESI) Calcd. for C₂₂H₂₁NO₃: 347, found [M+H]⁺=348. ¹H NMR (400 MHz, DMSO-d₆) δ 7.43-7.32 (m, 4H), 7.25 (d, 1H), 7.15-7.11 (m, 1H), 6.85 (d, 1H), 6.30 (s, 1H), 3.08 (s, 3H), 2.85 (s, 3H), 2.41-2.36 (m, 2H), 2.10 (s, 3H), 1.47-1.42 (m, 1H), 1.34-1.32 (m, 1H).

### Example 237: Synthesis of (trans)-2-(2-oxo-4-(o-tolyl)-2H-chromen-7-yl)cyclopropane-1-carboxamide

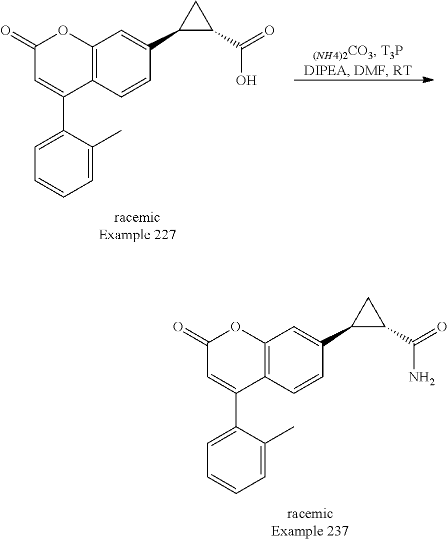 [SMILES: Cc1ccccc1-c1cc(=O)oc2cc([C@H]3C[C@@H]3C(=O)O)ccc12 ; Cc1ccccc1-c1cc(=O)oc2cc([C@H]3C[C@@H]3C(N)=O)ccc12]

Synthesis of (trans)-2-(2-oxo-4-(o-tolyl)-2H-chromen-7-yl)cyclopropane-1-carboxamide, Example 237: To a stirred solution of (trans)-2-(2-oxo-4-(o-tolyl)-2H-chromen-7-yl)cyclopropane-1-carboxylic acid (Example 227, 100 mg, 0.3 mmol) in DMF (2 mL), was added ammonium carbonate (300 mg, 3.1 mmol) and DIPEA (0.3 mL, 1.6 mmol) followed by the dropwise addition of T₃P (0.3 mL, 0.6 mmol, 50% in ethyl acetate) to the reaction mixture at 0° C. The reaction mixture was stirred at ambient temperature for 12 h., and quenched with ice water and extracted with 10% MeOH in DCM, washed with brine, dried over anhydrous Na₂SO₄, and concentrated under reduced pressure. The product was purified by RP prep-HPLC and lyophilized to afford (trans)-2-(2-oxo-4-(o-tolyl)-2H-chromen-7-yl)cyclopropane-1-carboxamide (Example 237, 30 mg). LCMS (ESI) Calcd. for C₂₀H₁₇NO: 319, found [M+H]⁺=320. ¹H NMR (400 MHz, DMSO-d₆) δ 7.62 (br s, 1H), 7.44-7.33 (m, 3H), 7.29 (d, 1H), 7.25 (d, 1H), 7.07 (t, 1H), 6.97 (s, 1H), 6.86 (d, 1H), 6.30 (s, 1H), 2.33 (br s, 1H), 2.09 (s, 3H), 1.94-1.91 (m, 1H), 1.42-1.37 (m, 1H), 1.34-1.31 (m, 1H).

### Example 238-239: Synthesis of racemic and chiral analogs of (trans)-2-(2-oxo-4-(o-tolyl)-2H-chromen-7-yl)cyclopropane-1-carboxamide

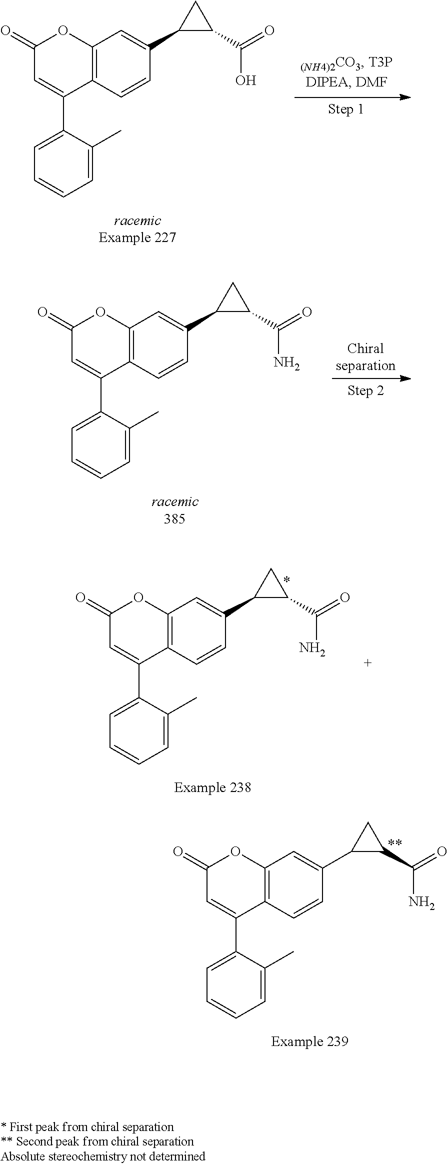

Synthesis of (rac)-2-(2-oxo-4-(o-tolyl)-2H-chromen-7-yl)cyclopropane-1-carboxamide, 385 [Step 1]: To a stirred solution of (rac)-2-(2-oxo-4-(o-tolyl)-2H-chromen-7-yl)cyclopropane-1-carboxylic acid (Example 227, 250 mg, 0.8 mmol) in DMF (4 mL), was added ammonium carbonate (750 mg, 7.8 mmol) and DIPEA (0.7 mL, 3.9 mmol). The reaction mixture was cooled in an ice bath and T₃P (1.0 mL, 1.6 mmol, 50% in ethyl acetate) was added dropwise. The reaction mixture was stirred at ambient temperature for 12 h. and was quenched with ice water. The mixture was extracted with 10% MeOH in dichloromethane, washed with brine, dried over anhydrous Na₂SO₄, and concentrated under reduce pressure. The product was purified by reverse phase prep-HPLC and lyophilized to afford (rac)-2-(2-oxo-4-(o-tolyl)-2H-chromen-7-yl)cyclopropane-1-carboxamide (385, 120 mg). LCMS (ESI) Calcd for C₂₀H₁₇NO₃: 319.3, found [M+H]⁺=320.2. ¹H NMR 400 MHz (DMSO-d₆) δ 7.61 (s, 1H), 7.43-7.38 (m, 2H), 7.36 (t, 1H), 7.29 (d, 1H), 7.25 (d, 1H), 7.07 (t, 1H), 6.97 (s, 1H), 6.86 (d, 1H), 2.33 (d, 1H), 2.09 (s, 3H), 1.94-1.91 (m, 1H), 1.42-1.37 (m, 1H), 1.34-1.31 (m, 1H).

Synthesis of chiral-(trans)-2-(2-oxo-4-(o-tolyl)-2H-chromen-7-yl)cyclopropane-1-carboxamide, Example 238 and Example 239 [Step 2]: (rac)-2-(2-oxo-4-(o-tolyl)-2H-chromen-7-yl)cyclopropane-1-carboxamide (385, 120 mg, 0.8 mmol) was separated by SFC chiral separation and lyophilized to afford Peak 1 as chiral-(trans)-2-(2-oxo-4-(o-tolyl)-2H-chromen-7-yl)cyclopropane-1-carboxamide (Example 238, 55 mg) and Peak 2 as chiral-(trans)-2-(2-oxo-4-(o-tolyl)-2H-chromen-7-yl)cyclopropane-1-carboxamide (Example 239, 50 mg). The absolute stereochemistry of these Examples was not determined.

Example 238: Chiral-(trans)-2-(2-oxo-4-(o-tolyl)-2H-chromen-7-yl)cyclopropane-1-carboxamide, Peak 1: LCMS (ESI) Calcd. for C₂₀H₁₇NO₃: 319, found [M+H]⁺=320. ¹H NMR (400 MHz, DMSO-d₆) δ 7.61 (s, 1H), 7.45-7.39 (m, 2H), 7.37 (t, 1H), 7.29 (d, 1H), 7.25 (d, 1H), 7.08 (t, 1H), 6.96 (s, 1H), 6.86 (d, 1H), 6.30 (s, 1H), 2.35-2.31 (m, 1H), 2.10 (s, 3H), 1.95-1.91 (m, 1H), 1.42-1.38 (m, 1H), 1.34-1.31 (m, 1H).

Example 239: Chiral-(trans)-2-(2-oxo-4-(o-tolyl)-2H-chromen-7-yl)cyclopropane-1-carboxamide: Peak 2: LCMS (ESI) Calcd. for C₂₀H₁₇NO₃: 319, found [M+H]⁺: 320. ¹H NMR (400 MHz, DMSO-d₆) δ 7.61 (s, 1H), 7.45-7.39 (m, 2H), 7.37 (t, 1H), 7.29 (d, 1H), 7.25 (d, 1H), 7.08 (t, 1H), 6.96 (s, 1H), 6.86 (d, 1H), 6.30 (s, 1H), 2.35-2.31 (m, 1H), 2.10 (s, 3H), 1.95-1.91 (m, 1H), 1.42-1.38 (m, 1H), 1.34-1.31 (m, 1H).

Chiral Prep method: SFC prep purification was performed on a PIC-SOLUTION-175 instrument using Chiralpak IG column (30×250 mm), 5μ operating at 35° C., at a flow rate of 70 mL/min. Mobile phase was a mixture of 60% super critical CO₂ and 40% of methanol. Run time was isocratic up to 20 minutes at an isobaric condition of 100 bar, and detection at 210 nm wavelength.

### Example 240-241: Synthesis of chiral and racemic analogs of (trans)-N,N-dimethyl-2-(2-oxo-4-(o-tolyl)-2H-chromen-7-yl)cyclopropane-1-carboxamide

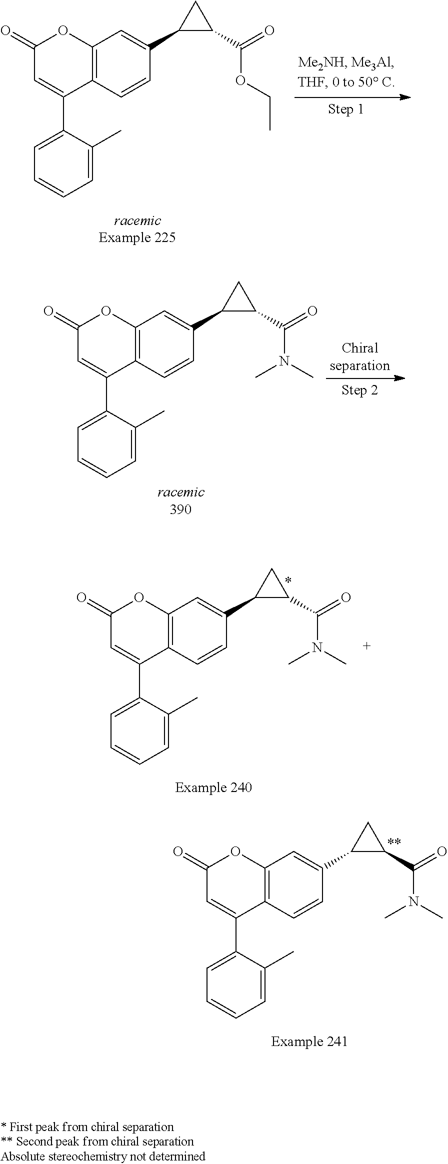

Synthesis of (rac)-N,N-dimethyl-2-(2-oxo-4-(o-tolyl)-2H-chromen-7-yl)cyclopropane-1-carboxamide, 390 [Step 1]: A stirred solution of ethyl (rac)-2-(2-oxo-4-(o-tolyl)-2H-chromen-7-yl)cyclopropane-1-carboxylate (Example 225, 300 mg, 0.9 mmol) in toluene (2 mL) was cooled to 0° C., and dimethylamine in THF (4.0 mL, 4.3 mmol, 2M) was added followed by trimethyl aluminum solution in toluene (2.0 mL, 1.7 mmol, 2M). The reaction mixture was stirred at 50° C. for 12 h., and quenched with ice-water and extracted with ethyl acetate (thrice). The combined organic extracts was washed with brine, dried over anhydrous Na₂SO₄, and concentrated under reduced pressure. The product was purified by reverse phase prep-HPLC and lyophilized to obtain (rac)-N,N-dimethyl-2-(2-oxo-4-(o-tolyl)-2H-chromen-7-yl)cyclopropane-1-carboxamide (390, 120 mg). LCMS (ESI) Calcd. for C₂₂H₂₁NO₃: 347, found [M+H]⁺=348. ¹H NMR 400 MHz (DMSO-d₆) δ 7.43-7.32 (m, 4H), 7.25 (d, 1H), 7.15-7.11 (m, 1H), 6.85 (d, 1H), 6.30 (s, 1H), 3.08 (s, 3H), 2.85 (s, 3H), 2.41-2.36 (m, 2H), 2.10 (s, 3H), 1.47-1.42 (m, 1H), 1.34-1.32 (m, 1H).

Synthesis of chiral-(trans)-N,N-dimethyl-2-(2-oxo-4-(o-tolyl)-2H-chromen-7-yl)cyclopropane-1-carboxamide, Example 240 and Example 241 [Step 2]: (rac)-N,N-dimethyl-2-(2-oxo-4-(o-tolyl)-2H-chromen-7-yl)cyclopropane-1-carboxamide (390, 120 mg, 0.3 mmol) was separated by SFC chiral separation and lyophilized to afford Peak 1 as chiral-(trans)-N,N-dimethyl-2-(2-oxo-4-(o-tolyl)-2H-chromen-7-yl)cyclopropane-1-carboxamide (Example 240, 51 mg) and Peak 2 as chiral-(trans)-N,N-dimethyl-2-(2-oxo-4-(o-tolyl)-2H-chromen-7-yl)cyclopropane-1-carboxamide (Example 241, 52 mg). The absolute stereochemistry of these Examples was not determined.

Example 240: chiral-(trans)-N,N-dimethyl-2-(2-oxo-4-(o-tolyl)-2H-chromen-7-yl)cyclopropane-1-carboxamide, Peak 1: LCMS (ESI) Calcd. for C₂₂H₂₁NO₃: 347, found [M+H]⁺=348. ¹H NMR (400 MHz, DMSO-d₆) δ 7.45-7.33 (m, 4H), 7.25 (d, 1H), 7.15 (t, 1H), 6.85 (d, 1H), 6.30 (s, 1H), 3.08 (s, 3H), 2.84 (s, 3H), 2.41-2.32 (m, 2H), 2.10 (s, 3H), 1.47-1.43 (m, 1H), 1.34 (t, 1H).

Example 241: chiral-(trans)-N,N-dimethyl-2-(2-oxo-4-(o-tolyl)-2H-chromen-7-yl)cyclopropane-1-carboxamide, Peak 2: LCMS (ESI) Calcd. for C₂₂H₂₁NO₃: 347, found [M+H]⁺=348. ¹H NMR (400 MHz, DMSO-d₆) δ 7.45-7.33 (m, 4H), 7.25 (d, 1H), 7.15 (t, 1H), 6.85 (d, 1H), 6.30 (s, 1H), 3.08 (s, 3H), 2.84 (s, 3H), 2.41-2.32 (m, 2H), 2.10 (s, 3H), 1.47-1.43 (m, 1H), 1.34 (t, 1H).

Chiral Prep method: SFC prep purification was performed on a PIC-SOLUTION-175 instrument using Chiralpak IG column (30×250 mm), 5μ operating at 35° C., at a flow rate of 70 mL/min. Mobile phase was a mixture of 45% super critical CO₂ and 55% of methanol. Run time was isocratic up to 20 minutes at an isobaric condition of 100 bar, and detection at 215 nm wavelength.

### Example 242: Synthesis of (R)-7-((1-(1,1-dioxidothiomorpholino)-1-oxopropan-2-yl)oxy)-4-(o-tolyl)-2H-chromen-2-one

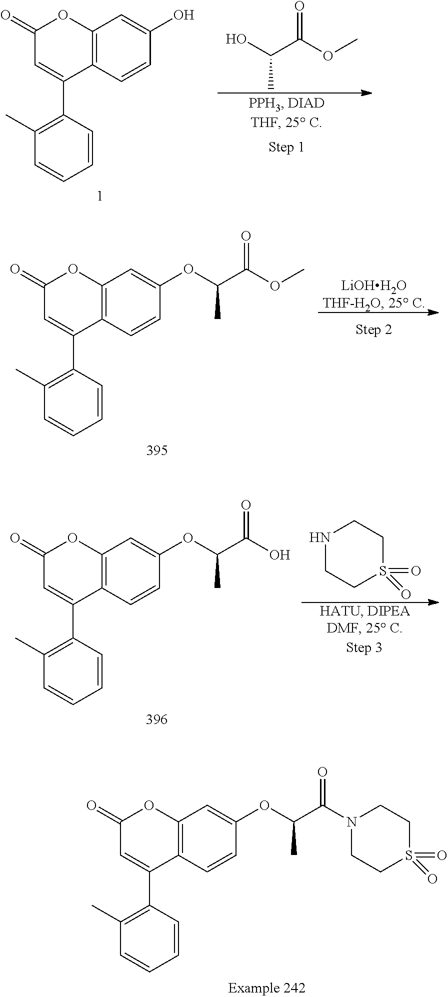

Synthesis of methyl(R)-2-((2-oxo-4-(o-tolyl)-2H-chromen-7-yl)oxy)propanoate, 395 [Step 1]: Diisopropyl azodicarboxylate (1.2 mL, 5.9 mmol) was added dropwise to an ice-cold solution of 7-hydroxy-4-(o-tolyl)-2H-chromen-2-one (1, 1.0 g, 3.9 mmol), methyl(S)-2-hydroxypropanoate (0.6 mL, 5.9 mmol), and triphenylphosphine (1.6 g, 5.9 mmol) in THF (20 mL). The reaction mixture was stirred at ambient temperature for 16 h., then concentrated under reduced pressure. The product was purified by column chromatography to afford methyl(R)-2-((2-oxo-4-(o-tolyl)-2H-chromen-7-yl)oxy)propanoate (395, 800 mg). LCMS (ESI) Calcd. for C₂₀H₁₈O₅: 338, found [M+H]⁺=339. ¹H NMR (400 MHz, DMSO-d₆) δ 7.43-7.39 (m, 2H), 7.37-7.33 (m, 1H), 7.25-7.23 (m, 1H), 7.02 (s, 1H), 6.90-6.84 (m, 2H), 6.21 (s, 1H), 5.22-5.20 (m, 1H), 3.70 (s, 3H), 2.12 (d, 3H), 1.54 (d, 3H).

Synthesis of (R)-2-((2-oxo-4-(o-tolyl)-2H-chromen-7-yl)oxy)propanoic acid, 396 [Step 2]: To a solution of methyl(R)-2-((2-oxo-4-(o-tolyl)-2H-chromen-7-yl)oxy)propanoate (395, 150 mg, 0.4 mmol) in a mixture of THF (2 mL)-water (0.5 mL) was added lithium hydroxide monohydrate (45 mg, 1.1 mmol), and the reaction mixture was stirred at ambient temperature for 16 h. The product was concentrated under reduced pressure and acidified with 1N aqueous HCl to pH=2, and extracted with ethyl acetate (twice). The combined organic extract was washed with brine, dried over anhydrous Na₂SO₄, filtered, and concentrated under reduced pressure to afford (R)-2-((2-oxo-4-(o-tolyl)-2H-chromen-7-yl)oxy)propanoic acid (396, 120 mg). LCMS (ESI) Calcd. for C₁₉H₁₆O₅: 324, found [M+H]⁺=325.

Synthesis of (R)-7-((1-(1,1-dioxidothiomorpholino)-1-oxopropan-2-yl)oxy)-4-(o-tolyl)-2H-chromen-2-one, Example 242: To a stirred solution of (R)-2-((2-oxo-4-(o-tolyl)-2H-chromen-7-yl)oxy)propanoic acid (396, 100 mg, 0.5 mmol) in DMF (3 mL) was added HATU (265 mg, 0.7 mmol) followed by N,N-diisopropylethylamine (0.2 mL, 1.2 mmol) at 0° C. After stirring for 10 min. at ambient temperature, thiomorpholine 1,1-dioxide (5, 75 mg, 0.5 mmol) was added and the reaction mixture was stirred at ambient temperature for 4 h. The reaction mixture was diluted with ice cold water, and extracted with ethyl acetate (twice). The combined organic extract was washed with brine and dried over anhydrous Na₂SO₄, filtered, and concentrated under reduced pressure. The product was purified by reverse phase prep-HPLC and lyophilized to afford (R)-7-((1-(1,1-dioxidothiomorpholino)-1-oxopropan-2-yl)oxy)-4-(o-tolyl)-2H-chromen-2-one (Example 242, 55 mg). LCMS (ESI) Calcd. for C₂₃H₂₃NO₆S: 441, found [M+H]⁺=442. ¹H NMR (400 MHz, DMSO-d₆) δ 7.45-7.33 (m, 2H), 7.36-7.33 (m, 1H), 7.25-7.22 (m, 1H), 7.05-7.04 (m, 1H), 6.90-6.88 (m, 1H), 6.83-6.79 (m, 1H), 6.15 (s, 1H), 5.49-5.44 (m, 1H), 3.95-3.94 (m, 4H), 3.16-3.15 (m, 4H), 2.14 (s, 3H), 1.52 (d, 3H).

### Example 243-244: Synthesis of 3-((4-(2,6-dimethylphenyl)-2-oxo-2H-chromen-7-yl)(methyl)amino)propanoic acid, 2-((4-(2,6-dimethylphenyl)-2-oxo-2H-chromen-7-yl)(methyl)amino)acetamide

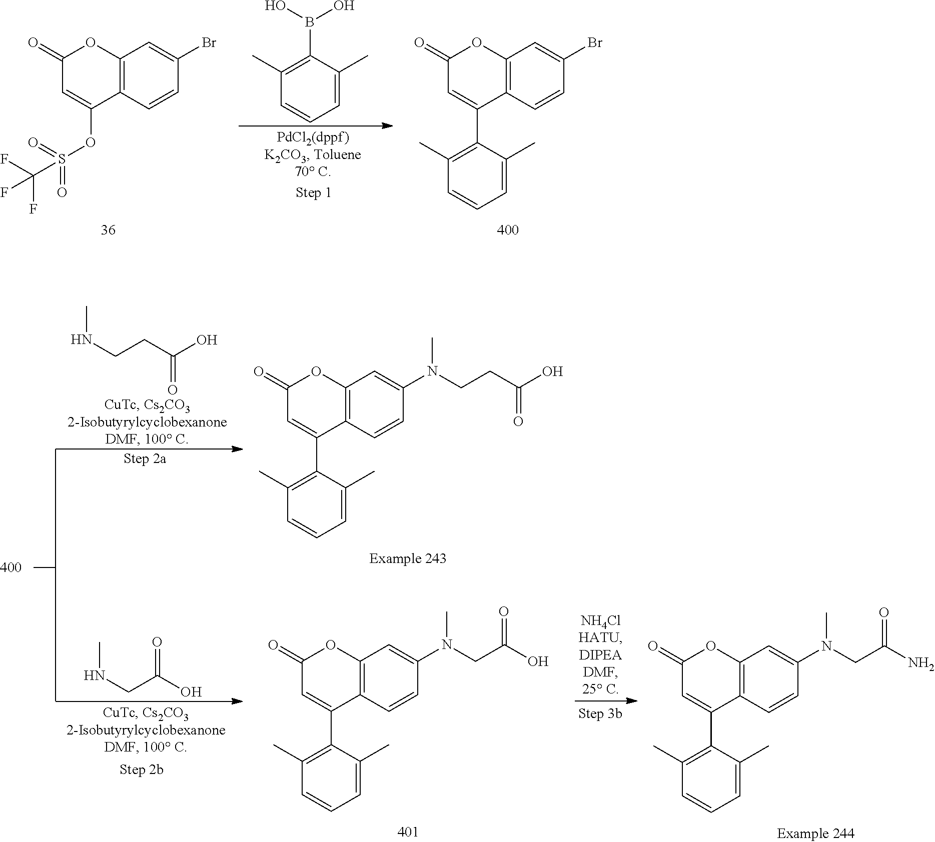

Synthesis of 7-bromo-4-(2,6-dimethylphenyl)-2H-chromen-2-one, 400 [Step 1]: A mixture of 7-bromo-2-oxo-2H-chromen-4-yl trifluoromethanesulfonate (36, 1 g, 2.7 mmol), (2,6-dimethylphenyl) boronic acid (360 mg, 2.4 mmol), and potassium carbonate (740 mg, 5.4 mmol) in toluene (2 mL) was degassed for 5 min. at ambient temperature. To this solution was added PdCl₂(dppf) (200 mg, 0.3 mmol) and the reaction mixture was heated to 70° C. After 4 h., the reaction mixture was cooled to ambient temperature, diluted with water and extracted with ethyl acetate. The collected organic extracts were washed with water, brine, dried over anhydrous Na₂SO₄, filtered, and concentrated under reduced pressure. The product was purified by column chromatography to afford 7-bromo-4-(2,6-dimethylphenyl)-2H-chromen-2-one (400, 350 mg). LCMS (ESI) Calcd. for C₁₇H₁₃BrO₂: 328, found [M+H]⁺=329. ¹H NMR (400 MHz, DMSO-d₆) δ 7.84-7.83 (m, 1H), 7.47-7.45 (m, 1H), 7.34-7.30 (m, 1H), 7.23-7.22 (m, 2H), 6.76 (d, 1H), 6.44 (s, 1H), 2.03 (s, 6H).

Synthesis of 3-((4-(2,6-dimethylphenyl)-2-oxo-2H-chromen-7-yl)(methyl)amino)propanoic acid, 243 [Step 2a]: A mixture of 7-bromo-4-(2,6-dimethylphenyl)-2H-chromen-2-one (400, 80 mg, 0.2 mmol), 3-(methylamino)propanoic acid (38 mg, 0.4 mmol), copper thiophene 2-carboxylate (10 mg, 49 μmol), cesium carbonate (158 mg, 0.5 mmol), 2-isobutyrylcyclohexanone (33 μL, 0.2 mmol) and DMF (1 mL) in a sealed tube was heated to 100° C. for 12 h. The reaction mixture was quenched with water and ethyl acetate. The water layer was collected, acidified up to pH 2-3 using 2N HCl, and extracted with 15% MeOH-dichloromethane. The combined organic extract was washed with brine, dried over anhydrous Na₂SO₄, filtered, and concentrated under reduced pressure. The product was purified by reverse phase prep-HPLC to afford 3-((4-(2,6-dimethylphenyl)-2-oxo-2H-chromen-7-yl)(methyl)amino)propanoic acid (400, 25 mg). LCMS (ESI) Calcd. for C₂₁H₂₁NO₄: 351, found [M+H]⁺=352. ¹H NMR (400 MHz, DMSO-d₆) δ 7.30-7.26 (m, 1H), 7.20-7.18 (m, 2H), 6.65-6.58 (m, 3H), 5.88 (s, 1H), 3.64 (t, 2H) 2.98 (s, 3H), 2.44 (t, 2H), 2.03 (s, 6H). 0620 Synthesis of N-(4-(2,6-dimethylphenyl)-2-oxo-2H-chromen-7-yl)-N-methylglycine, 401 [Step 2b]: A mixture of 7-bromo-4-(2,6-dimethylphenyl)-2H-chromen-2-one (400, 80 mg, 0.2 mmol) and methylglycine (32 mg, 0.4 mmol), copper thiophene 2-carboxylate (10 mg, 49 μmol), cesium carbonate (160 mg, 0.5 mmol), 2-isobutyrylcyclohexanone (33 μL, 0.2 mmol) and DMF (1 mL) in a sealed tube was heated to 100° C. for 12 h. The reaction mixture was quenched with water and ethyl acetate. The water layer was collected, acidified up to pH 2-3 using 2N HCl, and extracted using 15% MeOH-dichloromethane. The combined organic extract was washed with brine, dried over anhydrous Na₂SO₄, filtered, and concentrated under reduced pressure. The product was purified by reverse phase prep-HPLC to afford N-(4-(2,6-dimethylphenyl)-2-oxo-2H-chromen-7-yl)-N-methylglycine (400, 80 mg) LCMS (ESI) Calcd. for C₂₀H₁₉NO₄: 337, found [M+H]⁺=338.

Synthesis of 2-((4-(2,6-dimethylphenyl)-2-oxo-2H-chromen-7-yl)(methyl)amino)acetamide, Example 244 [Step 3b]: To a stirred solution of N-(4-(2,6-dimethylphenyl)-2-oxo-2H-chromen-7-yl)-N-methylglycine (400, 80 mg, 0.2 mmol) and ammonium chloride (190 mg, 3.6 mmol) in DMF (1 mL), was successively added HATU (150 mg, 0.4 mmol) and DIPEA (0.1 mL, 0.5 mmol), and the reaction mixture was stirred at 25° C. After 16 h., the reaction mixture was quenched with cold water, and extracted with ethyl acetate (twice). The organic layers were combined and washed with brine, dried over anhydrous Na₂SO₄, filtered, and concentrated under reduced pressure. The product was purified by reverse phase prep-HPLC to afford 2-((4-(2,6-dimethylphenyl)-2-oxo-2H-chromen-7-yl)(methyl)amino)acetamide (Example 244, 11 mg). LCMS (ESI) Calcd. for C₂₀H₂₀N₂O₃: 336, found [M+H]⁺=337. ¹H NMR (400 MHz, DMSO-d₆) δ 7.45 (s, 1H), 7.30-7.27 (m, 1H), 7.20-7.15 (m, 3H), 6.61-6.54 (m, 3H), 5.90 (s, 1H), 4.00 (s, 2H), 3.06 (s, 3H) 2.03 (s, 6H).

### Example 245-246: Synthesis of (R)-3-((4-(2,6-dimethylphenyl)-2-oxo-2H-chromen-7-yl)(methyl)amino)butanoic acid, and (S)-3-((4-(2,6-dimethylphenyl)-2-oxo-2H-chromen-7-yl)(methyl)amino)butanoic acid

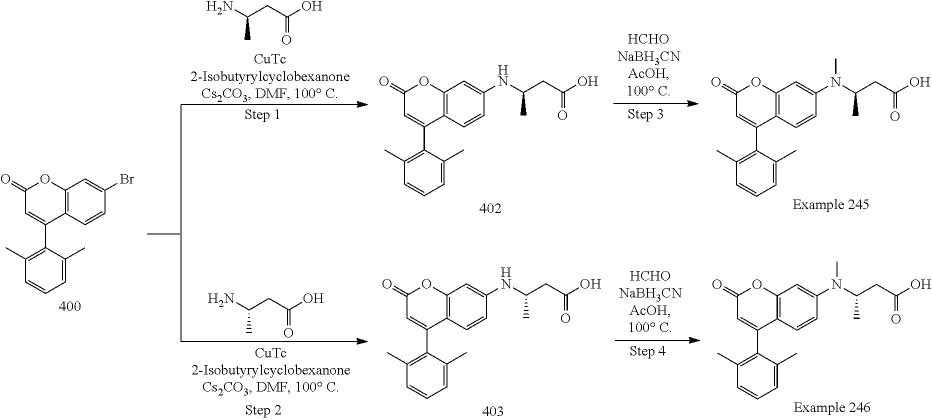

Synthesis of (R)-3-((4-(2,6-dimethylphenyl)-2-oxo-2H-chromen-7-yl)amino)butanoic acid, 402 [Step 1]: A mixture of 7-bromo-4-(2,6-dimethylphenyl)-2H-chromen-2-one (400, 80 mg, 0.2 mmol), (R)-3-aminobutanoic acid (40 mg, 0.4 mmol), cesium carbonate (160 mg, 0.5 mmol), copper thiophene 2-carboxylate (9.3 mg, 0.05 mmol), 2-isobutyrylcyclohexanone (33 mg, 0.2 mmol) and DMF (3 mL) in a sealed tube was flushed with argon and the reaction mixture was heated to 100° C. for 16 h. The reaction mixture was diluted with water and ethyl acetate, and the aqueous extracts was acidified using 2N HCl to pH 2-3 and extracted using 15% methanol in dichloromethane. The organic extract was washed with brine, dried over Na₂SO₄, filtered, and concentrated under reduced pressure to afford (R)-3-((4-(2,6-dimethylphenyl)-2-oxo-2H-chromen-7-yl)amino)butanoic acid (402, 80 mg). LCMS (ESI) Calcd. for C₂₁H₂₁NO₄: 351, found [M+H]⁺=352.

Synthesis of (S)-3-((4-(2,6-dimethylphenyl)-2-oxo-2H-chromen-7-yl)amino)butanoic acid, 403 [Step 2]: A mixture of 7-bromo-4-(2,6-dimethylphenyl)-2H-chromen-2-one (400, 80 mg, 0.2 mmol), (S)-3-aminobutanoic acid (5, 100 mg, 0.3 mmol), cesium carbonate (160 mg, 0.5 mmol), copper thiophene 2-carboxylate (9.3 mg, 0.05 mmol), 2-Isobutyrylcyclohexanone (35 mg, 0.2 mmol) and DMF (3 mL) in a sealed tube was flushed with argon and the reaction mixture was heated to 100° C. for 16 h. The reaction mixture was diluted with water and ethyl acetate, and the aqueous extract was acidified using 2N HCl to pH 2-3 and extracted using 15% methanol in dichloromethane. The organic extract was washed with brine, dried over Na₂SO₄, filtered, and concentrated under reduced pressure to afford(S)-3-((4-(2,6-dimethylphenyl)-2-oxo-2H-chromen-7-yl)amino)butanoic acid (403, 80 mg,). LCMS (ESI) Calcd. for C₂₁H₂₁NO₄: 351, found [M+H]⁺=352.

Synthesis of (R)-3-((4-(2,6-dimethylphenyl)-2-oxo-2H-chromen-7-yl)(methyl)amino)butanoic acid, Example 245 [Step 3]: To a stirred solution of (R)-3-((4-(2,6-dimethylphenyl)-2-oxo-2H-chromen-7-yl)amino)butanoic acid (402, 100 mg, 0.3 mmol) and formaldehyde (0.1 mL, 2.8 mmol, 37% in aqueous solution) in acetic acid (2 mL), was added sodium cyanoborohydride (18 mg, 0.3 mmol) at 0° C. The reaction mixture was stirred for 30 min. at 25° C., then concentrated under reduced pressure and quenched with ice water. The product was extracted using 15% methanol in dichloromethane (twice), and the organic extracts were dried over Na₂SO₄ and concentrated under reduced pressure. The product was purified by reverse phase prep-HPLC to afford (R)-3-((4-(2,6-dimethylphenyl)-2-oxo-2H-chromen-7-yl)(methyl)amino)butanoic acid (Example 245, 30 mg). LCMS (ESI) Calcd. for C₂₂H₂₃NO₄: 365, found [M+H]⁺=366. ¹H NMR (400 MHz, methanol-d₄) δ 7.27-7.23 (m, 1H), 7.17-7.15 (m, 2H), 6.79-6.71 (m, 3H), 5.87 (s, 1H), 4.60 (q, 1H), 2.86 (s, 3H), 2.67-2.61 (m, 1H), 2.56-2.51 (m, 1H), 2.07 (s, 6H), 3.16 (d, 3H).

Synthesis of (S)-3-((4-(2,6-dimethylphenyl)-2-oxo-2H-chromen-7-yl)(methyl)amino)butanoic acid, Example 246 [Step 4]: To a stirred solution of (S)-3-((4-(2,6-dimethylphenyl)-2-oxo-2H-chromen-7-yl)amino)butanoic acid (403, 100 mg, 0.3 mmol) and formaldehyde (0.1 mL, 2.85 mmol, 37% in aqueous solution) in acetic acid (2 mL) was added sodium cyanoborohydride (55 mg, 0.8 mmol) at 0° C. The reaction mixture was stirred for 30 min. at 25° C. The reaction mixture was concentrated under reduced pressure, quenched with ice water and extracted using 15% methanol in dichloromethane (twice). The combined organic extracts were dried over anhydrous Na₂SO₄ and concentrated under reduced pressure. The product was purified by reverse phase pre-HPLC and lyophilized to afford(S)-3-((4-(2,6-dimethylphenyl)-2-oxo-2H-chromen-7-yl)(methyl)amino)butanoic acid (Example 246, 15 mg). LCMS (ESI) Calcd. for C₂₂H₂₃NO₄: 365, found [M+H]⁺=366. ¹H NMR (400 MHz, methanol-d₄) δ 7.27-7.23 (m, 1H), 7.17-7.15 (m, 2H), 6.79-6.71 (m, 3H), 5.87 (s, 1H), 4.60 (q, 1H), 2.86 (s, 3H), 2.67-2.61 (m, 1H), 2.56-2.51 (m, 1H), 2.07 (s, 6H), 3.16 (d, 3H).

### Example 247: Synthesis of N-methyl-N-(2-(2-(methyl(2-oxo-4-(o-tolyl)-2H-chromen-7-yl)amino)acetamido)ethyl)acetamide

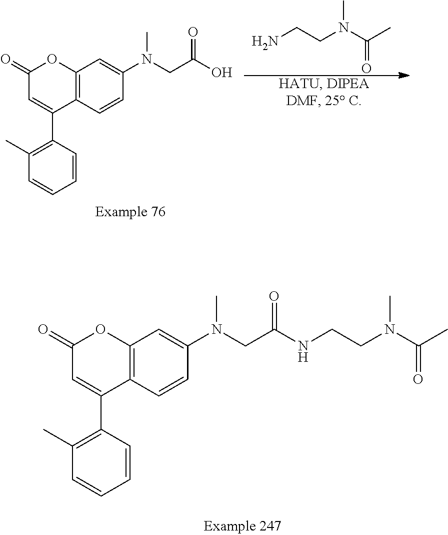

Synthesis of N-methyl-N-(2-(2-(methyl(2-oxo-4-(o-tolyl)-2H-chromen-7-yl)amino)acetamido)ethyl)acetamide, Example 247: To a stirred solution of N-methyl-N-(2-oxo-4-(o-tolyl)-2H-chromen-7-yl)glycine (Example 76, 150 mg, 0.5 mmol) and N-(2-aminoethyl)-N-methylacetamide hydrochloride (75 mg, 0.5 mmol) was added HATU (265 mg, 0.7 mmol) followed by DIPEA (0.2 mL, 1.2 mmol), and the reaction mixture was stirred at 25° C. After 16 h., the reaction mixture was quenched with cold water, and extracted with 20% methanol in dichloromethane (twice). The combined organic extracts were washed with brine, dried over anhydrous Na₂SO₄, filtered, and concentrated under reduced pressure. Purification of the compound by reverse phase prep-HPLC, followed by lyophilization afforded N-methyl-N-(2-(2-(methyl(2-oxo-4-(o-tolyl)-2H-chromen-7-yl)amino)acetamido)ethyl)acetamide (Example 247, 98 mg). LCMS (ESI) Calcd. for C₂₄H₂₇N₃O₄: 421, found [M+H]⁺=422. ¹H NMR (400 MHz, methanol-d₄) δ 7.39-7.29 (m, 3H), 7.18-7.17 (m, 1H), 6.89-6.87 (m, 1H), 6.65-6.59 (m, 2H), 5.95-5.94 (m, 1H), 4.08-4.03 (m, 2H), 3.48-3.37 (m, 4H), 3.14 (s, 3H), 3.01-2.88 (m, 3H), 2.14 (s, 3H), 2.04-1.98 (m, 3H).

### Example 248: Synthesis of (R)-4-(2,6-dimethylphenyl)-7-((1-(1,1-dioxidothiomorpholino)-1-oxopropan-2-yl)oxy)-2H-chromen-2-one

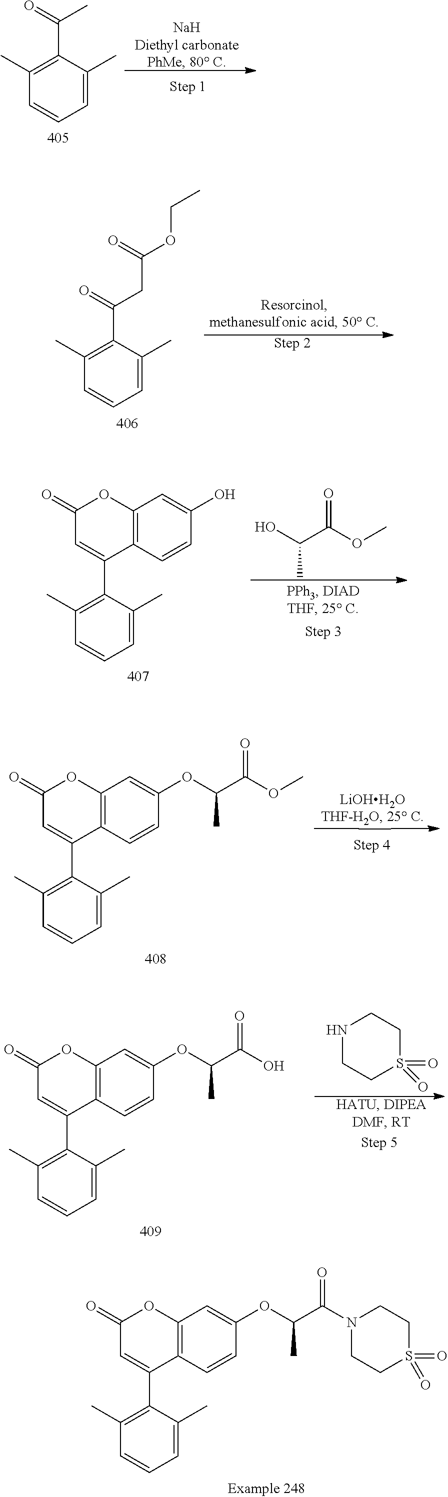

Synthesis of ethyl 3-(2,6-dimethylphenyl)-3-oxopropanoate, 406 [Step 1]: To an ice-cold solution of 1-(2,6-dimethylphenyl) ethan-1-one (405, 5.0 g, 33.7 mmol) and diethyl carbonate (15 mL, 135.0 mmol) in toluene (100 mL) was added NaH (2.70 g, 60% in mineral oil, 67.5 mmol). After warming to ambient temperature, the reaction vessel was placed into an oil-bath preheated to 80° C. After heating for 16 h., the reaction mixture was allowed to cool to ambient temperature, and then cooled in an ice-bath. The reaction mixture was quenched with cold water, and then extracted with ethyl acetate. The organic extract was washed with brine, dried over anhydrous Na₂SO₄, filtered, and concentrated under reduced pressure to afford ethyl 3-(2,6-dimethylphenyl)-3-oxopropanoate (406, 6.0 g). LCMS (ESI) Calcd. for C₁₃H₁₆O₃: 220, found [M+H]⁺=221.

Synthesis of 4-(2,6-dimethylphenyl)-7-hydroxy-2H-chromen-2-one, 407 [Step 2]: Ethyl 3-(2,6-dimethylphenyl)-3-oxopropanoate (406, 3.0 g, 13.6 mmol) and resorcinol (1.50 g, 13.6 mmol) were mixed in methanesulfonic acid (7.0 mL, 108.0 mmol), and the reaction mixture was stirred at 50° C. After 16 h, the reaction mixture was cooled to ambient temperature. Cold water was added, and the resulting precipitate was collected by filtration. The product was washed several times with cold water followed by hexanes, and dried under reduced pressure to afford 4-(2,6-dimethylphenyl)-7-hydroxy-2H-chromen-2-one (407, 2.5 g). LCMS (ESI) Calcd. for C₁₇H₁₄O₃: 266, found [M+H]⁺=267. ¹H NMR (400 MHz, DMSO-d₆) δ 7.94-7.92 (m, 1H), 7.34-7.31 (m, 1H), 7.20-7.18 (m, 2H), 6.97-6.94 (m, 1H), 6.87-6.86 (m, 1H), 6.26 (s, 1H), 4.17 (br s, 1H), 2.23 (s, 6H).

Synthesis of methyl(R)-2-((4-(2,6-dimethylphenyl)-2-oxo-2H-chromen-7-yl)oxy)propanoate, 408 [Step 3]: To an ice-cold solution of 4-(2,6-dimethylphenyl)-7-hydroxy-2H-chromen-2-one (407, 1.0 g, 3.8 mmol), methyl(S)-2-hydroxypropanoate (0.5 mL, 5.6 mmol) and triphenylphosphine (1.5 g, 5.6 mmol) in THF (20 mL), was added diisopropyl azodicarboxylate (1.1 mL, 5.6 mmol) and the reaction mixture was stirred at ambient temperature. After 16 h., the reaction mixture was concentrated under reduced pressure, and purified by Combi-Flash column chromatography to afford methyl(R)-2-((4-(2,6-dimethylphenyl)-2-oxo-2H-chromen-7-yl)oxy)propanoate (408, 1.0 g). LCMS (ESI) Calcd. for C₂₁H₂₀O₅: 352, found [M+H]⁺=353.

Synthesis of (R)-2-((4-(2,6-dimethylphenyl)-2-oxo-2H-chromen-7-yl)oxy)propanoic acid, 409 [Step 4]: To a solution of methyl(R)-2-((4-(2,6-dimethylphenyl)-2-oxo-2H-chromen-7-yl)oxy)propanoate (408, 200 mg, 0.57 mmol) in THF (4 mL)-water (1 mL) was added lithium hydroxide monohydrate (35 mg, 0.8 mmol), and the reaction mixture was stirred at ambient temperature. After 16 h., the reaction mixture was concentrated under reduced pressure, acidified with 1N aqueous HCl to pH 2, and extracted with 20% methanol in dichloromethane (twice). The combined organic extract was washed with brine, dried over anhydrous Na₂SO₄, filtered, and concentrated under reduced pressure to afford (R)-2-((4-(2,6-dimethylphenyl)-2-oxo-2H-chromen-7-yl)oxy)propanoic acid (409, 160 mg). The product was used in the next step without further purification. LCMS (ESI) Calcd. for C₂₀H₁₈O₅: 338, found [M+H]⁺=339.

Synthesis of (R)-4-(2,6-dimethylphenyl)-7-((1-(1,1-dioxidothiomorpholino)-1-oxopropan-2-yl)oxy)-2H-chromen-2-one, Example 248 [Step 5]: To a solution of (R)-2-((4-(2,6-dimethylphenyl)-2-oxo-2H-chromen-7-yl)oxy)propanoic acid (409, 160 mg, 0.5 mmol) and thiomorpholine 1,1-dioxide (7, 77 mg, 0.6 mmol) in DMF (5 mL), was added HATU (270 mg, 0.7 mmol) followed by DIPEA (0.2 mL, 1.2 mmol), and the reaction mixture was stirred at ambient temperature. After 16 h., the reaction mixture was quenched with cold water, and extracted with ethyl acetate (twice). The combined organic extract was washed with brine, dried over anhydrous Na₂SO₄, filtered, and concentrated under reduced pressure. The product was purified by reverse phase prep-HPLC followed by lyophilization to afford (R)-4-(2,6-dimethylphenyl)-7-((1-(1,1-dioxidothiomorpholino)-1-oxopropan-2-yl)oxy)-2H-chromen-2-one (Example 248, 80 mg). LCMS (ESI) Calcd. for C₂₄H₂₅NO₆S: 455, found [M+H]⁺=456. ¹H NMR (400 MHz, DMSO-d₆) (at 100° C.) δ 8.05-8.03 (m, 1H), 7.33-7.31 (m, 1H), 7.21-7.19 (m, 2H), 7.12-7.09 (m, 2H), 6.27 (s, 1H), 5.51 (q, 1H), 3.98-3.95 (m, 4H), 3.16-3.14 (m, 4H), 2.27 (s, 6H), 1.53 (d, 3H).

### Examples 249-250: Synthesis of racemic and chiral analogs of 1-(methyl((2-oxo-4-(o-tolyl)-2H-chromen-7-yl)methyl)carbamoyl)piperidine-2-carboxylic acid

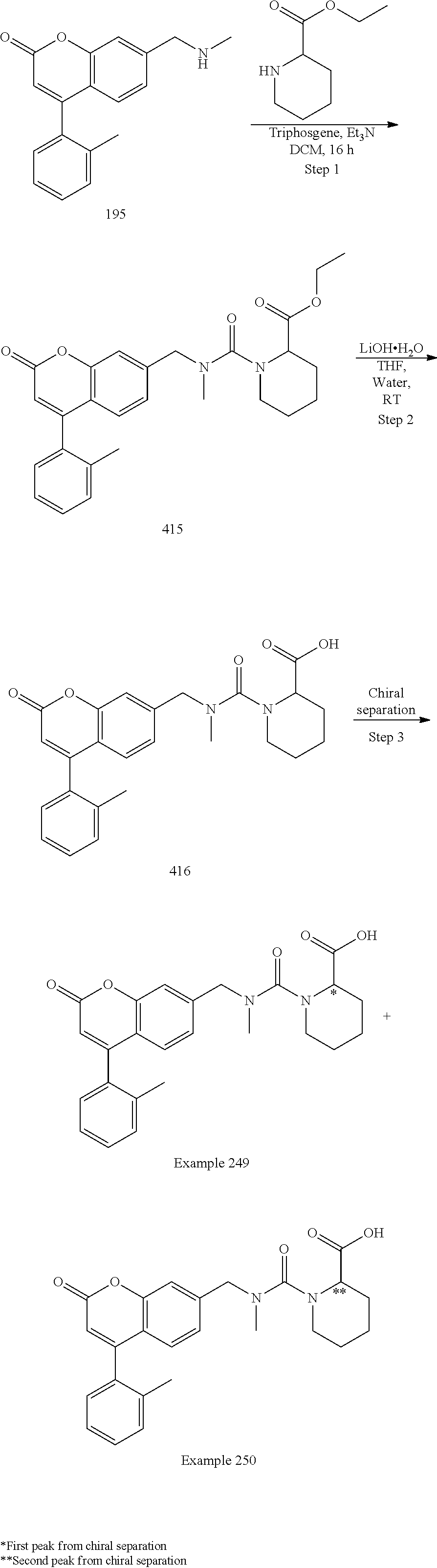

Synthesis of ethyl 1-(methyl((2-oxo-4-(o-tolyl)-2H-chromen-7-yl)methyl)carbamoyl)piperidine-2-carboxylate, 415 [Step 1]: To a stirred solution of ethyl piperidine-2-carboxylate hydrochloride (200 mg, 1.0 mmol) in dichloromethane (5 mL) was added triethylamine (0.4 mL, 3.1 mmol) followed by triphosgene (400 mg, 1.3 mmol) at 0° C. and the reaction mixture was stirred for 20 min. To this reaction mixture was added 7-((methylamino)methyl)-4-(o-tolyl)-2H-chromen-2-one (195, 315 mg, 1.1 mmol) and the mixture was stirred at ambient temperature for 16 h. The reaction mixture was quenched with water and extracted with dichloromethane (twice). The combined organic extracts were collected and washed with water, brine, dried over anhydrous Na₂SO₄, filtered, and concentrated under reduced pressure to afford ethyl 1-(methyl((2-oxo-4-(o-tolyl)-2H-chromen-7-yl)methyl)carbamoyl)piperidine-2-carboxylate (415, 450 mg). LCMS (ESI) Calcd. for C₂₇H₃₀N₂O₅: 462; found [M+H]⁺=463.

Synthesis of 1-(methyl((2-oxo-4-(o-tolyl)-2H-chromen-7-yl)methyl)carbamoyl)piperidine-2-carboxylic acid, 416 [Step 2]: To a stirred solution of ethyl 1-(methyl((2-oxo-4-(o-tolyl)-2H-chromen-7-yl)methyl)carbamoyl)piperidine-2-carboxylate (415, 400 mg, 0.9 mmol) in THF (8 mL) and water (2 mL), was added LiOH H₂O (110 mg, 2.6 mmol) and the reaction mixture was stirred at ambient temperature for 4 h. The reaction mixture was concentrated under reduced pressure, diluted with water, and acidified with 1N HCl to pH 4. The mixture was extracted with ethyl acetate and washed with water, brine, dried over anhydrous Na₂SO₄, filtered, and concentrated under reduced pressure. The product was purified by reverse phase prep-HPLC and lyophilized to afford 1-(methyl((2-oxo-4-(o-tolyl)-2H-chromen-7-yl)methyl)carbamoyl)piperidine-2-carboxylic acid (416, 100 mg).

Synthesis of chiral analogs of 1-(methyl((2-oxo-4-(o-tolyl)-2H-chromen-7-yl)methyl)carbamoyl)piperidine-2-carboxylic acid, Example 249 and Example 250 [Step 3]: 1-(methyl((2-oxo-4-(o-tolyl)-2H-chromen-7-yl)methyl)carbamoyl)piperidine-2-carboxylic acid (415, 100 mg) was purified by chiral prep-HPLC and lyophilized to afford Peak 1 as 1-(methyl((2-oxo-4-(o-tolyl)-2H-chromen-7-yl)methyl)carbamoyl)piperidine-2-carboxylic acid (Example 249, 12 mg) and Peak 2 as 1-(methyl((2-oxo-4-(o-tolyl)-2H-chromen-7-yl)methyl)carbamoyl)piperidine-2-carboxylic acid (Example 250, 12 mg).

Example 250: 1-(methyl((2-oxo-4-(o-tolyl)-2H-chromen-7-yl)methyl)carbamoyl)piperidine-2-carboxylic acid, Peak 1: LCMS (ESI) Calcd. for C₂₅H₂₆N₂O₅: 434, found [M+H]⁺=435. ¹H NMR (400 MHz, DMSO-d₆) δ 12.4 (br s, 1H), 7.46-7.39 (m, 2H), 7.36-7.34 (m, 2H), 7.26 (d, 1H), 7.18 (d, 1H), 6.94 (d, 1H), 6.35 (s, 1H), 4.42 (s, 2H), 4.14 (s, 1H), 3.24-3.20 (m, 2H), 2.74 (s, 3H), 2.12 (s, 3H), 1.90-1.87 (m, 1H), 1.70 (br s, 1H), 1.54 (br s, 2H), 1.45-1.40 (m, 2H).

Example 251: 1-(methyl((2-oxo-4-(o-tolyl)-2H-chromen-7-yl)methyl)carbamoyl)piperidine-2-carboxylic acid, Peak 2: LCMS (ESI) Calcd. for C₂₅H₂₆N₂O₅: 434, found [M+H]⁺=435. ¹H NMR (400 MHz, DMSO-d₆) δ 12.4 (br s, 1H), 7.46-7.40 (m, 2H), 7.36-7.34 (m, 2H), 7.26 (d, 1H), 7.18 (d, 1H), 6.94 (d, 1H), 6.35 (s, 1H), 4.42 (s, 2H), 4.14 (s, 1H), 3.24-3.19 (m, 2H), 2.74 (s, 3H), 2.12 (s, 3H), 1.90-1.87 (m, 1H), 1.70 (br s, 1H), 1.54 (br s, 2H), 1.45-1.40 (m, 2H).

Prep HPLC chiral method: Chiral separation was performed on an Agilent 1200 series instrument with column CHIRALPAK IG (250×21 mm), 5μ, operating at ambient temperature with a flow rate of 21.0 mL/min. The mobile phase was a mixture of 70% hexane, 15% DCM, 15% IPA and 0.1% TFA, held isocratic for up to 30 min. with wavelength of 280 nm.

The absolute stereochemistry was determined by performing a reaction using 416 and ethyl (S)-piperidine-2-carboxylate. The product isolated was ethyl (S)-1-(methyl((2-oxo-4-(o-tolyl)-2H-chromen-7-yl)methyl)carbamoyl)piperidine-2-carboxylate, which was converted into final corresponding acid. The chiral HPLC retention time of S-isomer matched with Peak 2, Example 250.

### Example 251: Synthesis of N-(methyl-d₃)-2-(methyl(2-oxo-4-(o-tolyl)-2H-chromen-7-yl)amino)acetamide

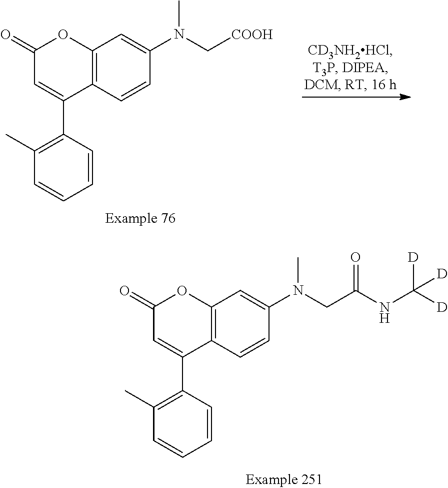 [SMILES: Cc1ccccc1-c1cc(=O)oc2cc(N(C)CC(=O)O)ccc12 ; [2H]C([2H])([2H])NC(=O)CN(C)c1ccc2c(-c3ccccc3C)cc(=O)oc2c1]

Synthesis of N-(methyl-d₃)-2-(methyl(2-oxo-4-(o-tolyl)-2H-chromen-7-yl)amino)acetamide, Example 251: To a stirred solution of N-methyl-N-(2-oxo-4-(o-tolyl)-2H-chromen-7-yl)glycine (Example 76, 50 mg, 0.15 mmol) in dichloromethane (2 mL) was added DIPEA (0.15 mL, 0.8 mmol) and trideuteriomethanamine hydrochloride (13 mg, 0.19 mmol), and the reaction mixture was cooled to 0° C. To this cold reaction mixture was added T₃P (0.14 mL, 0.23 mmol, 50% EtOAc) and the reaction mixture was allowed to warm to ambient temperature and stirred for 16 h. The reaction mixture was diluted with dichloromethane, washed with water, brine, dried over anhydrous Na₂SO₄, filtered, and concentrated under reduced pressure. The product was purified by reverse phase prep-HPLC and lyophilized to afford N-(methyl-d₃)-2-(methyl(2-oxo-4-(o-tolyl)-2H-chromen-7-yl)amino)acetamide (Example 251, 31 mg). LCMS (ESI) Calcd. for C₂₀H₁₇D₃N₂O₃: 339, found [M+H]⁺=340. ¹H NMR (400 MHz, DMSO-d₆) δ 7.86 (s, 1H), 7.41-7.31 (m, 3H), 7.20 (d, 1H), 6.72 (d, 1H), 6.59-6.56 (m, 2H), 5.92 (s, 1H), 4.01 (s, 2H), 3.06 (s, 3H), 2.11 (s, 3H).

### Biological/Biochemical Evaluation

General protocol for in vitro analysis of compounds 0640 The inhibitory activity of the compounds of the present invention against POLRMT were determined by assays based on Bergbrede, T., et al., “An adaptable high-throughput technology enabling the identification of specific transcription modulators,” SLAC Discov., 22, 378-386 (2017).

The ability of some compounds of the present invention to inhibit POLRMT were determined in a homogeneous TR-FRET Assay using high-throughput screening in a 384-well plate format. This method is used to monitor the activity of mitochondrial transcription through measurement of its product, a 407 bp long RNA transcript. Detection of the product is facilitated by hybridization of two DNA-oligonucleotide probes to specific and adjacent sequences within the RNA product sequence. Upon annealing of the probes, two fluorophores are coupled directly to an acceptor nucleotide probe (ATTO647, 5′), or introduced via a coupled streptavidin with a biotinylated donor nucleotide probe (Europium cryptate) that is brought into sufficient proximity to serve as a fluorescence-donor-acceptor pair. Thus, a FRET signal at 665 nm is generated upon excitation at 340 nm.

Proteins used as transcription factors (POLRMT: NP_005026.3, TFAM: NP 003192.1, TFB2M: NP_071761.1) are diluted from their stocks to working concentrations of 1 μM, 20 μM and 4 μM respectively, in a dilution buffer containing 20 mM Tris-HCl (pH 8.0), 200 mM NaCl, 10% (v/v) glycerol, 1 mM Dithiothreitol (DTT), 0.5 mM EDTA.

DNA template is a pUC18 plasmid with the mitochondrial light strand promotor sequence (1-477) cloned between HindIII and BamHI sites. The DNA template is restriction linearized proximal to the promotor 3′-end (pUC-LSP). 0644 The reaction mixture (10 μL) containing 7.5 nM POLRMT, 15 nM of TFB2M, 30 nM of TFAM, 0.5 nM of DNA template and 500 μM nucleotide triphosphate mix (NTPs) in a reaction buffer (containing 10 mM Tris-HCl (pH 7.5), 10 mM MgCl₂, 40 mM NaCl, 10 mM DTT, 0.005% (w/v) Tween-20, 160 units/ml Rnase inhibitor and 0.1 mg/mL BSA) are dispensed to compounds in microplates, using a Thermo Multidrop® dispenser, and incubated at 37° C. in a VWR INCU-Line incubator for 60 minutes after mixing. No nucleotide triphosphate mix is added to negative control samples. Microplates with compounds to be tested in the assay are prepared from 10 mM compound stocks in 100% DMSO, equal amounts of DMSO without any compound are added to positive control and negative control samples.

During the incubation, a mix of the detection reagents is prepared in a buffer such that the enzymatic reaction is terminated due to chelating of Mg-ions and increased ionic strength, containing 50 mM Tris-HCl (pH 7.5), 700 mM NaCl, 20 mM EDTA, and 0.01% (w/v) Tween-20. Europium-streptavidin is pre-incubated with a 200-fold molar excess of a random sequence oligonucleotide to block unspecific binding of oligo, for two hours at ambient temperature in the dark. Afterwards, the blocked Europium-streptavidin is kept on ice until use.

At the end of the enzymatic reaction time, 5 μL detection oligo mix in the detection buffer is added, and assay plates are mixed and kept at ambient temperature for one hour, protected from light. The concentration of the Acceptor nucleotide oligo (e.g., ATTO647N-5′-ACAAAGAACCCTAACACCAG-3′) and Donor nucleotide oligo (e.g., bio-5′-AACACATCTCT(-bio)GCCAAACCCCA-bio-3′) in each assay well is 1 nM, and 3 nM, respectively.

After incubation with oligo mix, 5 μL of pre-blocked Europium-streptavidin reagent is dispensed to each assay well, assay plates are again mixed and kept at ambient temperature for one hour, protected from light.

The generated signal is measured with BMG Pherastar microtiter plate reader with a TRF light unit, using excitation at 340 nm, an integration time of 200 μs, and a delay time of 100 μs, before detection at 620 nm and 665 nm. The ratio of donor- and acceptor-fluorescence is used as a measure of the generated transcript product (i.e. enzymatic activity).

The IC₅₀ values are summarized in Table 1.

TABLE 1

| Examples | IC₅₀ (μM) |
|---|---|
| Example 01 | 0.4 |
| Example 02 | 2 |
| Example 03 | 19 |
| Example 04 | >100 |
| Example 05 | 4 |
| Example 06 | 3 |
| Example 07 | 7 |
| Example 08 | 1 |
| Example 09 | 10 |
| Example 10 | 0.4 |
| Example 11 | 24 |
| Example 12 | >100 |
| Example 13 | 10 |
| Example 14 | 18 |
| Example 15 | 2 |
| Example 16 | 1 |
| Example 18 | 0.2 |
| Example 19 | 24 |
| Example 20 | 0.8 |
| Example 21 | 49 |
| Example 22 | 25 |
| Example 23 | 19 |
| Example 25 | 0.4 |
| Example 26 | 0.4 |
| Example 27 | 0.1 |
| Example 28 | 0.8 |
| Example 29 | >100 |
| Example 30 | >100 |
| Example 31 | 0.4 |
| Example 32 | 0.4 |
| Example 33 | 0.3 |
| Example 34 | 2 |
| Example 35 | 2 |
| Example 36 | 2 |
| Example 37 | 1 |
| Example 38 | 0.6 |
| Example 39 | 0.7 |
| Example 40 | 2 |
| Example 41 | 1 |
| Example 42 | 6 |
| Example 43 | 13 |
| Example 44 | 12 |
| Example 45 | 0.05 |
| Example 46 | 0.05 |
| Example 47 | 0.4 |
| Example 48 | 3 |
| Example 49 | 1 |
| Example 50 | 2 |
| Example 51 | 0.3 |
| Example 52 | 0.2 |
| Example 53 | 2 |
| Example 54 | 0.9 |
| Example 55 | 0.07 |
| Example 56 | 0.2 |
| Example 57 | 0.3 |
| Example 58 | 3 |
| Example 59 | 2 |
| Example 60 | 8 |
| Example 61 | 0.9 |
| Example 62 | 0.08 |
| Example 63 | 3 |
| Example 64 | 0.3 |
| Example 65 | 17 |
| Example 66 | 0.5 |
| Example 67 | 6 |
| Example 68 | 0.6 |
| Example 69 | 5 |
| Example 70 | 0.04 |
| Example 71 | 6 |
| Example 72 | 3 |
| Example 73 | 3 |
| Example 74 | 0.7 |
| Example 75 | 0.03 |
| Example 76 | 0.2 |
| Example 77 | 2 |
| Example 78 | 44 |
| Example 79 | 2 |
| Example 80 | 0.1 |
| Example 81 | 1 |
| Example 82 | 5 |
| Example 83 | 0.06 |
| Example 84 | 32 |
| Example 85 | 0.3 |
| Example 86 | 5 |
| Example 87 | 9 |
| Example 88 | 0.09 |
| Example 89 | 0.1 |
| Example 90 | 8 |
| Example 91 | 20 |
| Example 92 | 0.2 |
| Example 93 | 17 |
| Example 94 | 0.2 |
| Example 95 | 4 |
| Example 96 | 2 |
| Example 97 | 0.3 |
| Example 98 | 14 |
| Example 99 | 0.4 |
| Example 100 | 21 |
| Example 101 | 10 |
| Example 102 | 3 |
| Example 103 | 5 |
| Example 104 | 0.3 |
| Example 105 | 0.5 |
| Example 106 | 0.2 |
| Example 107 | 3 |
| Example 108 | 0.04 |
| Example 109 | 5 |
| Example 110 | 8 |
| Example 111 | 64 |
| Example 112 | 27 |
| Example 113 | 12 |
| Example 114 | 36 |
| Example 115 | 18 |
| Example 116 | 45 |
| Example 117 | 65 |
| Example 118 | 15 |
| Example 119 | 2 |
| Example 120 | 21 |
| Example 121 | 0.3 |
| Example 122 | 5 |
| Example 123 | 16 |
| Example 124 | 3 |
| Example 125 | 0.1 |
| Example 126 | 0.05 |
| Example 127 | 0.2 |
| Example 128 | 0.03 |
| Example 129 | 7 |
| Example 130 | 0.4 |
| Example 131 | 8 |
| Example 132 | 1 |
| Example 133 | 0.7 |
| Example 134 | 0.2 |
| Example 135 | 9 |
| Example 136 | 0.2 |
| Example 137 | 0.9 |
| Example 138 | 2 |
| Example 139 | 0.1 |
| Example 140 | 0.9 |
| Example 141 | 0.02 |
| Example 142 | 0.02 |
| Example 143 | 0.03 |
| Example 144 | 0.1 |
| Example 145 | 4 |
| Example 146 | 0.3 |
| Example 147 | 0.3 |
| Example 148 | 2 |
| Example 149 | 1 |
| Example 150 | 2 |
| Example 151 | 3 |
| Example 152 | 0.3 |
| Example 153 | 0.2 |
| Example 154-1 | 1 |
| Example 154-2 | 0.5 |
| Example 155-1 | 0.5 |
| Example 155-2 | 0.4 |
| Example 156 | 0.2 |
| Example 157 | 2 |
| Example 158 | 0.8 |
| Example 159 | >100 |
| Example 160 | >100 |
| Example 161 | >100 |
| Example 162 | >100 |
| Example 163 | 0.5 |
| Example 164 | 4 |
| Example 165 | 3 |
| Example 166 | 5 |
| Example 167 | 0.1 |
| Example 168 | 0.09 |
| Example 169 | 3 |
| Example 170 | >100 |
| Example 171 | >100 |
| Example 172 | >100 |
| Example 173 | 73 |
| Example 174 | 47 |
| Example 175 | >100 |
| Example 176 | 75 |
| Example 177 | 0.05 |
| Example 178 | 0.05 |
| Example 179 | 0.1 |
| Example 180 | 0.2 |
| Example 181 | >100 |
| Example 182 | 12 |
| Example 183 | 7 |
| Example 184 | 3 |
| Example 185 | 0.7 |
| Example 186 | 28 |
| Example 187 | 0.1 |
| Example 188 | 0.08 |
| Example 189 | 0.9 |
| Example 190 | 0.8 |
| Example 191 | 7 |
| Example 192 | 2 |
| Example 193 | 0.1 |
| Example 194 | 0.3 |
| Example 195 | 0.05 |
| Example 196 | 42 |
| Example 198 | 2 |
| Example 199 | 0.8 |
| Example 200 | 3 |
| Example 201 | 2 |
| Example 202 | 2 |
| Example 203 | 2 |
| Example 204 | 0.03 |
| Example 205 | >100 |
| Example 206 | >100 |
| Example 207 | 17 |
| Example 208 | 3 |
| Example 209 | 0.3 |
| Example 210 | 3 |
| Example 211 | 1 |
| Example 212 | 0.6 |
| Example 213 | 21 |
| Example 216 | 0.6 |
| Example 217 | 0.09 |
| Example 218 | 0.2 |
| Example 219 | 0.2 |
| Example 220 | 0.1 |
| Example 221 | 2 |
| Example 222 | 2 |
| Example 223 | 0.8 |
| Example 224 | 0.3 |
| Example 225 | 0.7 |
| Example 226 | 0.4 |
| Example 227 | 3 |
| Example 228 | 0.2 |
| Example 229 | 0.1 |
| Example 230 | 0.2 |
| Example 231 | 0.4 |
| Example 232 | 2 |
| Example 233 | 5 |
| Example 234 | 3 |
| Example 235 | 2 |
| Example 236 | 7 |
| Example 237 | 2 |
| Example 238 | 3 |
| Example 239 | 0.9 |
| Example 240 | 8 |
| Example 241 | 5 |

### General Protocol for In Vivo AML (Acute Myeloid Leukemia) Efficacy Experiment

### Determination of Maximum Tolerated Dose of Test Compound

Immunocompromised mice (6-10-week-old, female NSG mice, strain NOD.Cg-Prkdc^{scid} Il2rg^{tm1Wj1}/Szj, Jackson Laboratories) are treated orally with test compound ranging from 75 to 150 mg/kg, once or twice per day for the duration of 14 days. Total body weight is measured, and the general condition of mice is monitored routinely. Mice with severe symptoms and moribund are excluded from study. Submental blood collection method (no anesthesia) is used for all samplings. Plasma levels of test compound are determined at intervals ranging from 0.5 to 4 hours post first and last doses in all dosing groups. From these data pharmacokinetic analysis are conducted.

### In Vivo Efficacy Study in AML Mouse Model

MV4-11 AML cell lines (ATCC) are labelled with luciferase tag by viral transduction procedure (MV4-11-luc).

For an AML cell line xenograft efficacy experiment, female NSG mice are given intravenously ˜1×10⁷ MV4-11-luc cells. Mice are flux sorted and randomized into treatment groups 14 days post transplantation. Mice are then treated with vehicle (50 mM Na₂HPO₄), or test compound at a tolerable dose determined from the above study, once or twice per day for 21 days. Tumor progression/regression is monitored by imaging of mice using luciferin as a substrate (150 mg/kg). Images are taken on a total of 9 time points i.e., one flux sort and once weekly to end date (8 time points). Imaging is performed under anesthesia and using in vivo imaging equipment IVIS. The treatment efficacy is also measured based on proportion of human AML cells, determined by flow cytometry analysis of viable human CD45 positive cell population in peripheral blood of mice one week post last dose. Plasma levels of test compound are determined at intervals ranging from 0.5 to 4 hours post last dose. Animals are monitored individually, and total body weight is measured routinely. The endpoint of the experiment is moribundity. In addition, mice demonstrating tumor-associated symptoms including impairment of hind limb function, ocular proptosis, and weight loss are considered for euthanasia. The remaining mice are euthanized on day 60 of the study.

1. A compound, or a pharmaceutically acceptable salt thereof, according to formula (I): 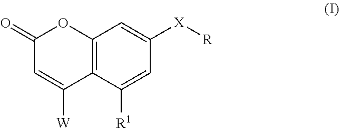
wherein;
X is C(R³)(R³), C₃-C₆ cycloalkyl, C(R³)(R³)O, C(R³)(R³)OC(O), C(R³)═C(R³), N(R⁴), C(R³)(R³)N(R³), C(O)(NR³), or C(O);
W is C₆-C₁₂ aryl or 5-12 membered heteroaryl, either of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium;
R is hydrogen, NR³R⁴, C(O)NR³R⁴, C₁-C₄ alkylcarboxyl, C(O)R³, carboxyl, CH₂CH₂R², hydroxyl, or C₁-C₆ alkoxyl optionally substituted with one or more groups, each independently selected from the group consisting of hydroxyl, fluoro, C₁-C₄ alkoxyl, NR³R⁴, C₁-C₄ alkylcarboxyl, carboxyl, and C(O)NR³R⁴,
or R is NR³C(O)R³, C₃-C₆ cycloalkyl optionally substituted with C(O)NR³R³, or C₄-C₆ heterocyclic,
or R is C₁-C₆ alkyl optionally substituted with one or more groups, each independently selected from the group consisting of hydroxyl, fluoro, cyano, C₁-C₄ alkoxyl, C₁-C₄ haloalkoxyl, phenyl, NR³R⁴, C₁-C₄ alkylcarboxyl, carboxyl, CR⁵R⁶—CO₂R⁷, C(O)NR³R⁴, C(O)NR³SO₂R³, NR³C(O)R³, C₃-C₆ cycloalkyl, 5- to 6-membered heterocyclic ring optionally substituted with R³, and heteroaryl optionally substituted with C₁-C₄ alkyl, hydroxyl, or oxo,
or R is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, and C₁-C₄ alkoxyl;
R¹ is hydrogen, hydroxyl, cyano, C₁-C₃ alkyl optionally substituted with one or more fluoro, C₃-C₄ cycloalkyl, or methoxyl optionally substituted with one to three fluoro;
R² is C₆-C₁₀ aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R² is C₁-C₄ haloalkoxyl, OR³, NR³R⁴, C(O)R³, or C(O)NR³R⁴;
each R³ is independently hydrogen, or C₃-C₆ cycloalkyl, C₄-C₆ heterocyclic, C₁-C₄ alkyl, each optionally substituted with one or more fluoro or carboxyl;
each R⁴ is independently R³, C(O) C₁-C₄ alkyl optionally substituted with one or more fluoro groups, C(O)NHC₁-C₄ alkyl optionally substituted with one or more fluoro groups, or C₁-C₄ alkyl optionally substituted with one or more deuterium, hydroxyl, fluoro, C₁-C₃ alkoxyl, NR³R³, carboxyl, or C(O)NR³R³;
or if R³ and R⁴ are attached to the same nitrogen atom, R³ and R⁴ together with their connecting nitrogen form a 4- to 6-membered heterocyclic ring optionally containing another heteroatom that is N, O, or S, and such heterocyclic ring is optionally substituted with one or more groups each independently selected from the group consisting of fluoro, chloro, cyano, carboxyl, C(O)NR³R³, NR³R⁴, NR¹C(O)R³, S(O)₂R³, S(O)₂NR³R³, C₁-C₄ alkylcarboxylate, and C₁-C₄ alkyl optionally substituted with hydroxyl, C₁-C₄ alkoxy, or NR¹C(O)R³;
R⁵ and R⁶ together with their connecting carbon form a 3- to 5-membered cyclic or heterocyclic ring; and
R⁷ is H or NH₂.

2. The compound according to claim 1, or a pharmaceutically acceptable salt thereof,
wherein;
X is C(R³)(R³), C₃-C₆ cycloalkyl, C(R³)(R³)O, C(R³)(R³)OC(O), C(R³)═C(R³), N(R⁴), C(R³)(R³)N(R³), C(O)(NR³), or C(O);
W is C₆ aryl optionally substituted with one or more groups, each independently selected from the group consisting of fluoro, chloro, C₁ alkoxyl, and C₁ alkyl optionally substituted with one or more deuterium;
R is hydrogen, NR³R⁴, C(O)NR³R⁴, C₁-C₂ alkylcarboxyl, C(O)R³, carboxyl, hydroxyl, C₁ alkoxyl, NR³C(O)R³, C₆ aryl, or C₃-C₈ cycloalkyl optionally substituted with C(O)NR³R³,
or R is C₁-C₈alkyl optionally substituted with one or more groups, each independently selected from the group consisting of hydroxyl, cyano, C₁ alkoxyl, C₁ haloalkoxyl, phenyl, NR³R⁴, C₁-C₄ alkylcarboxyl, carboxyl, CR⁵R⁶—CO₂R⁷, C(O)NR³R⁴, C(O)NR³SO₂R³, NR³C(O)R³, C₃ cycloalkyl, 5- to 6-membered heterocyclic ring optionally substituted with R³, and heteroaryl optionally substituted with C₁-C₄ alkyl, hydroxyl, or oxo;
R¹ is hydrogen, hydroxyl, or C₁ alkyl;
each R³ is independently hydrogen, or C₆ cycloalkyl, C₆ heterocyclic, C₁-C₃ alkyl, each optionally substituted with carboxyl;
each R⁴ is independently R³, or C₁-C₃ alkyl optionally substituted with one or more deuterium, hydroxyl, fluoro, C₁ alkoxyl, NR³R³, carboxyl, or C(O)NR³R³;
or if R³ and R⁴ are attached to the same nitrogen atom, R³ and R⁴ together with their connecting nitrogen form a 4- to 6-membered heterocyclic ring optionally containing another heteroatom that is O, and such heterocyclic ring is optionally substituted with one or more groups each independently selected from the group consisting of cyano, carboxyl, C(O)NR³R³, NR³R⁴, NR¹C(O)R³, S(O)₂R³, S(O)₂NR³R³, C₁-C₂ alkylcarboxylate, and C₁ alkyl optionally substituted with hydroxyl, C₁ alkoxy, or NR¹C(O)R³;
R⁵ and R⁶ together with their connecting carbon form a 3- to 5-membered cyclic or heterocyclic ring; and
R⁷ is H or NH₂.

3. The compound according to claim 2, or a pharmaceutically acceptable salt thereof,
wherein;
X is C(R³)(R³), C₃ cycloalkyl, C(R³)═C(R³), N(R⁴), or (R³)(R³)N(R³);
W is C₆ aryl optionally substituted with one or more groups, each independently selected from the group consisting of fluoro, chloro, and C₁ alkyl;
R is C(O)NR³R⁴, C₁-C₂ alkylcarboxyl, C(O)R³, carboxyl, or C₁ alkoxyl,
or R is C₁-C₄ alkyl optionally substituted with one or more groups, each independently selected from the group consisting of C₁ alkoxyl, phenyl, C₁-C₄ alkylcarboxyl, carboxyl, C(O)NR³R⁴, C₃ cycloalkyl, 5-membered heterocyclic, and heteroaryl optionally substituted with C₁ alkyl, hydroxyl, or oxo;
each R³ is independently hydrogen, or C₆ cycloalkyl, C₆ heterocyclic, C₁-C₃ alkyl, each optionally substituted with carboxyl;
each R⁴ is independently R³, or C₁-C₂ alkyl optionally substituted with one or more hydroxyl, fluoro, C₁ alkoxyl, or C(O)NR³R³;
or if R³ and R⁴ are attached to the same nitrogen atom, R³ and R⁴ together with their connecting nitrogen form a 5- to 6-membered heterocyclic ring optionally containing another heteroatom that is O, and such heterocyclic ring is optionally substituted with one or more groups each independently selected from the group consisting of cyano, carboxyl, C(O)NR³R³, S(O)₂R³, and S(O)₂NR³R³.

4. The compound according to claim 1, or a pharmaceutically acceptable salt thereof,
wherein
X is —N(R⁴)—;
W is C₆ aryl substituted with C₁ alkyl;
R is C₂ alkyl substituted with C(O)NR³R⁴;
R¹ is hydrogen; and
R³ and R⁴ together with their connecting nitrogen form a 6-membered heterocyclic ring substituted with carboxyl.

5. The compound according to claim 1, or a pharmaceutically acceptable salt thereof,
wherein
X is C(R³)(R³);
W is C₆ aryl substituted with one or more groups, each independently selected from the group consisting of fluoro, chloro, and C₁ alkyl;
R is C₂ alkyl substituted with C(O)NR³R⁴;
R¹ is hydrogen; and
R³ and R⁴ together with their connecting nitrogen form a 6-membered heterocyclic ring substituted with carboxyl.

6. The compound according to claim 1, or a pharmaceutically acceptable salt thereof,
wherein
X is —N(R⁴)—;
W is C₆ aryl substituted with C₁ alkyl;
R is C₁-C₄ alkyl substituted with one group selected from the group consisting of carboxyl and C(O)NR³R⁴;
R¹ is hydrogen;
each R³ is independently hydrogen or C₁ alkyl; and
each R⁴ is independently R³, or C₁-C₂ alkyl optionally substituted with hydroxyl or C(O)NR³R³.

7. A pharmaceutical composition comprising a compound according to claim 2, and a pharmaceutically acceptable excipient.

8. A pharmaceutical composition comprising a compound according to claim 3, and a pharmaceutically acceptable excipient.

9. A compound, or a pharmaceutically acceptable salt thereof, selected from the group consisting of:

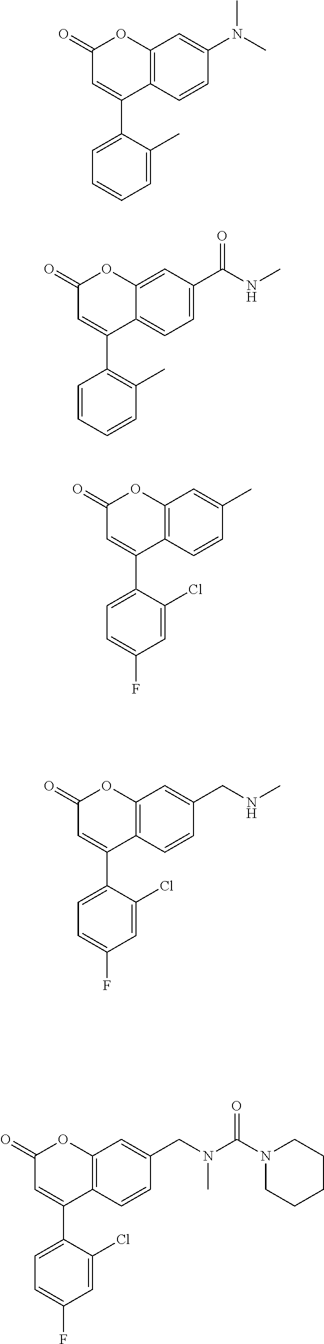 [SMILES: Cc1ccccc1-c1cc(=O)oc2cc(N(C)C)ccc12 ; CNC(=O)c1ccc2c(-c3ccccc3C)cc(=O)oc2c1 ; Cc1ccc2c(-c3ccc(F)cc3Cl)cc(=O)oc2c1 ; CNCc1ccc2c(-c3ccc(F)cc3Cl)cc(=O)oc2c1 ; CN(Cc1ccc2c(-c3ccc(F)cc3Cl)cc(=O)oc2c1)C(=O)N1CCCCC1]

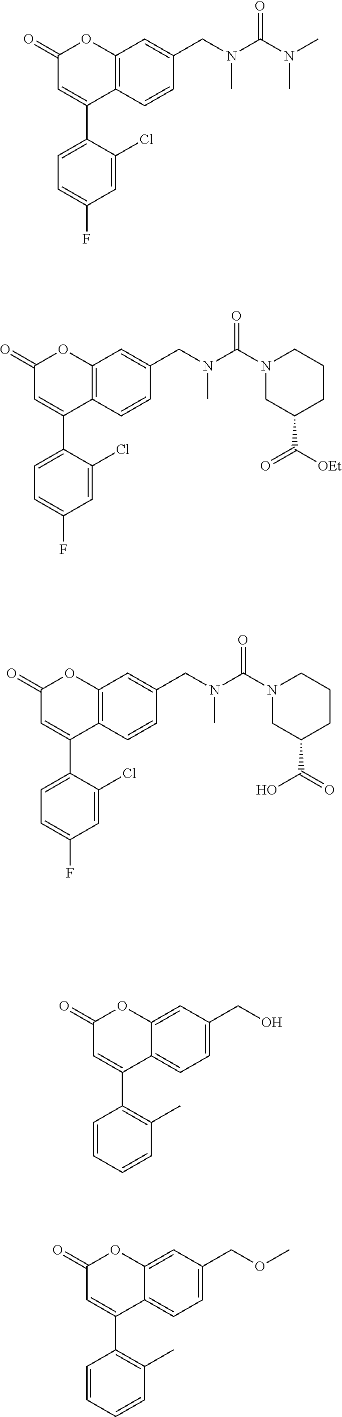 [SMILES: CN(C)C(=O)N(C)Cc1ccc2c(-c3ccc(F)cc3Cl)cc(=O)oc2c1 ; CCOC(=O)[C@H]1CCCN(C(=O)N(C)Cc2ccc3c(-c4ccc(F)cc4Cl)cc(=O)oc3c2)C1 ; CN(Cc1ccc2c(-c3ccc(F)cc3Cl)cc(=O)oc2c1)C(=O)N1CCC[C@H](C(=O)O)C1 ; Cc1ccccc1-c1cc(=O)oc2cc(CO)ccc12 ; COCc1ccc2c(-c3ccccc3C)cc(=O)oc2c1]

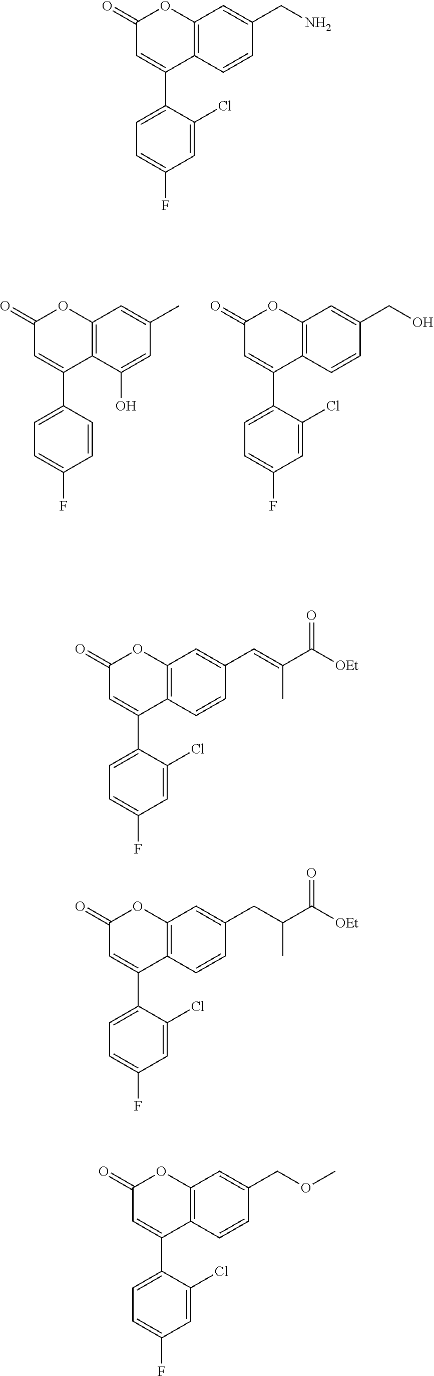

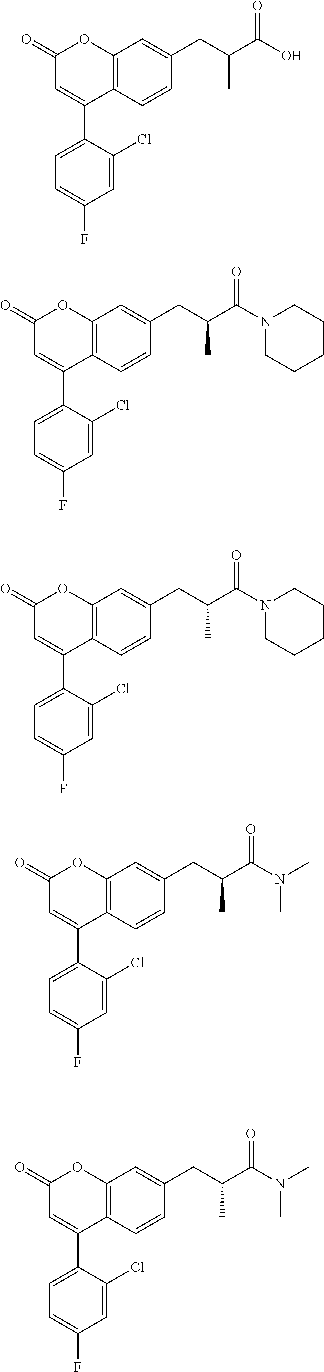 [SMILES: CC(Cc1ccc2c(-c3ccc(F)cc3Cl)cc(=O)oc2c1)C(=O)O ; C[C@@H](Cc1ccc2c(-c3ccc(F)cc3Cl)cc(=O)oc2c1)C(=O)N1CCCCC1 ; C[C@H](Cc1ccc2c(-c3ccc(F)cc3Cl)cc(=O)oc2c1)C(=O)N1CCCCC1 ; C[C@@H](Cc1ccc2c(-c3ccc(F)cc3Cl)cc(=O)oc2c1)C(=O)N(C)C ; C[C@H](Cc1ccc2c(-c3ccc(F)cc3Cl)cc(=O)oc2c1)C(=O)N(C)C]

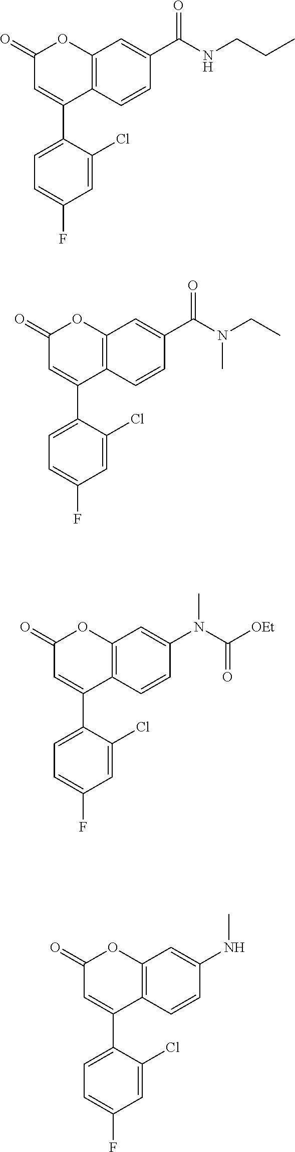 [SMILES: CCCNC(=O)c1ccc2c(-c3ccc(F)cc3Cl)cc(=O)oc2c1 ; CCN(C)C(=O)c1ccc2c(-c3ccc(F)cc3Cl)cc(=O)oc2c1 ; CCOC(=O)N(C)c1ccc2c(-c3ccc(F)cc3Cl)cc(=O)oc2c1 ; CNc1ccc2c(-c3ccc(F)cc3Cl)cc(=O)oc2c1]

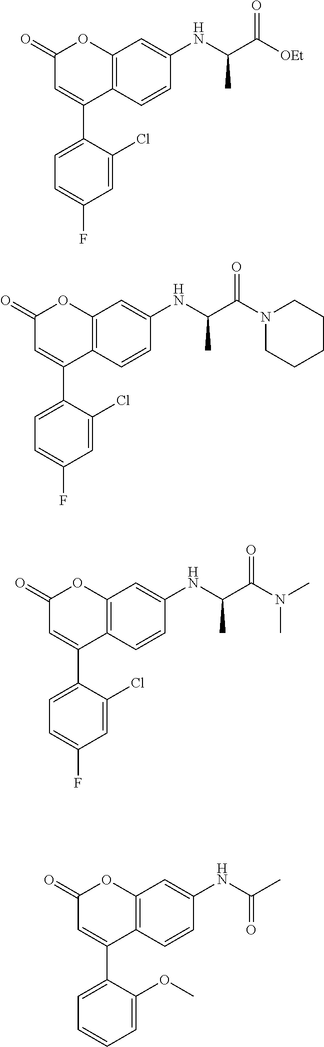 [SMILES: CCOC(=O)[C@@H](C)Nc1ccc2c(-c3ccc(F)cc3Cl)cc(=O)oc2c1 ; C[C@@H](Nc1ccc2c(-c3ccc(F)cc3Cl)cc(=O)oc2c1)C(=O)N1CCCCC1 ; C[C@@H](Nc1ccc2c(-c3ccc(F)cc3Cl)cc(=O)oc2c1)C(=O)N(C)C ; COc1ccccc1-c1cc(=O)oc2cc(NC(C)=O)ccc12]

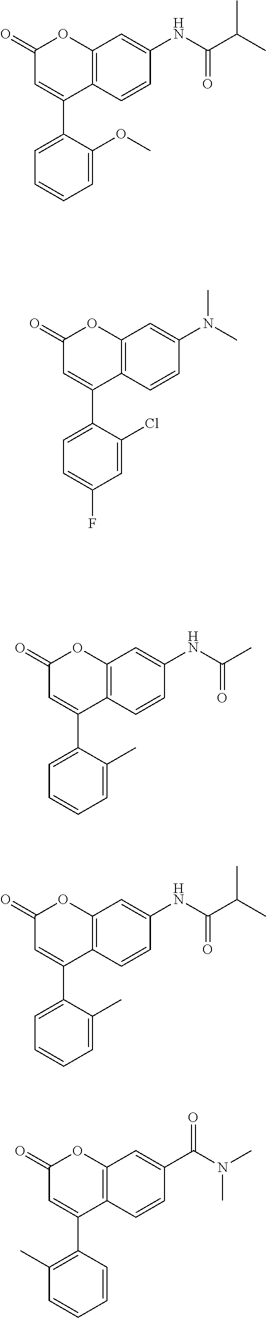 [SMILES: COc1ccccc1-c1cc(=O)oc2cc(NC(=O)C(C)C)ccc12 ; CN(C)c1ccc2c(-c3ccc(F)cc3Cl)cc(=O)oc2c1 ; CC(=O)Nc1ccc2c(-c3ccccc3C)cc(=O)oc2c1 ; Cc1ccccc1-c1cc(=O)oc2cc(NC(=O)C(C)C)ccc12 ; Cc1ccccc1-c1cc(=O)oc2cc(C(=O)N(C)C)ccc12]

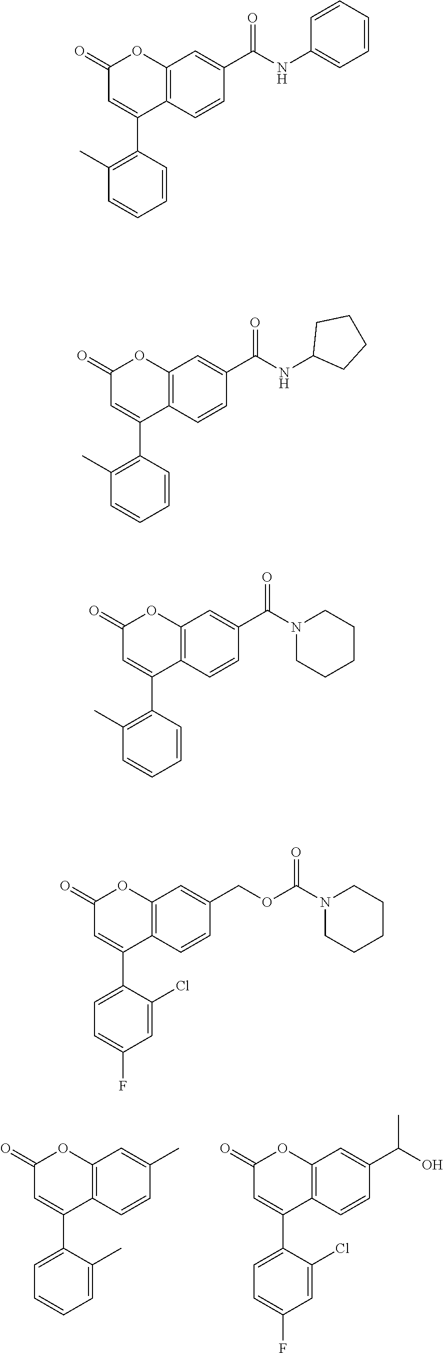

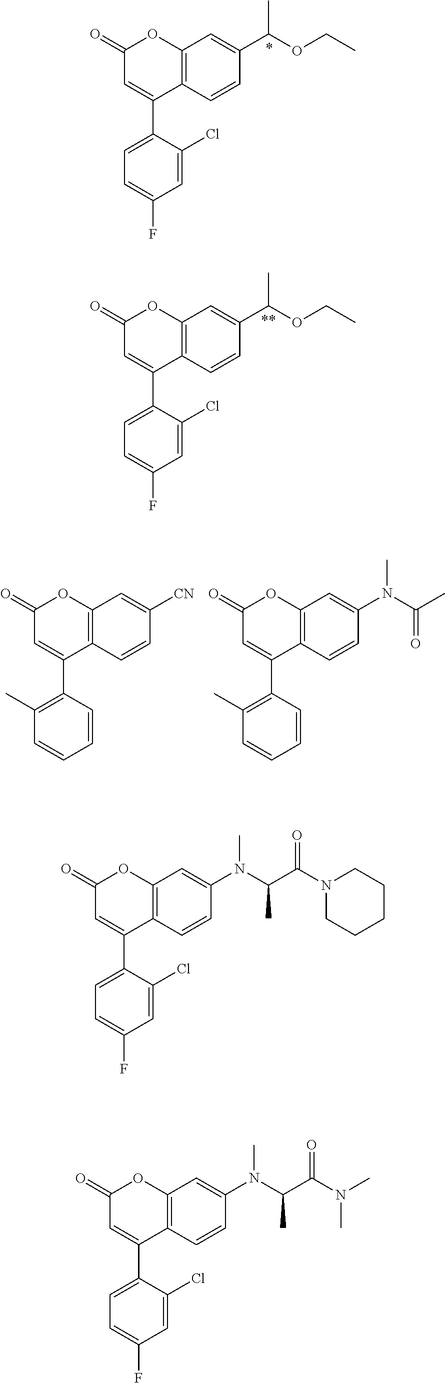

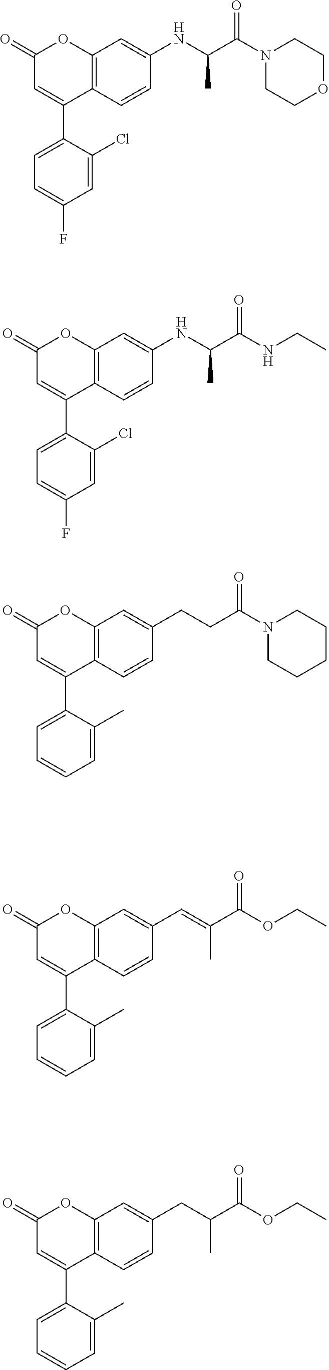 [SMILES: C[C@@H](Nc1ccc2c(-c3ccc(F)cc3Cl)cc(=O)oc2c1)C(=O)N1CCOCC1 ; CCNC(=O)[C@@H](C)Nc1ccc2c(-c3ccc(F)cc3Cl)cc(=O)oc2c1 ; Cc1ccccc1-c1cc(=O)oc2cc(CCC(=O)N3CCCCC3)ccc12 ; CCOC(=O)/C(C)=C/c1ccc2c(-c3ccccc3C)cc(=O)oc2c1 ; CCOC(=O)C(C)Cc1ccc2c(-c3ccccc3C)cc(=O)oc2c1]

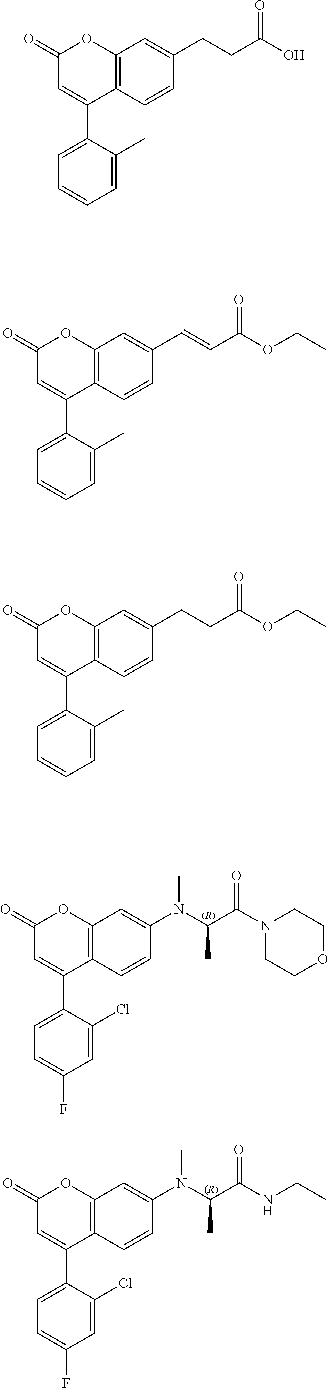

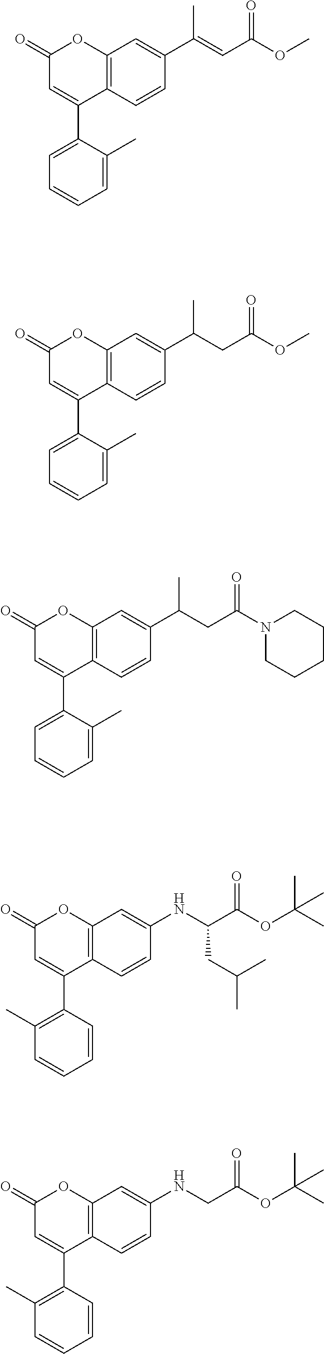 [SMILES: COC(=O)/C=C(\C)c1ccc2c(-c3ccccc3C)cc(=O)oc2c1 ; COC(=O)CC(C)c1ccc2c(-c3ccccc3C)cc(=O)oc2c1 ; Cc1ccccc1-c1cc(=O)oc2cc(C(C)CC(=O)N3CCCCC3)ccc12 ; Cc1ccccc1-c1cc(=O)oc2cc(N[C@@H](CC(C)C)C(=O)OC(C)(C)C)ccc12 ; Cc1ccccc1-c1cc(=O)oc2cc(NCC(=O)OC(C)(C)C)ccc12]

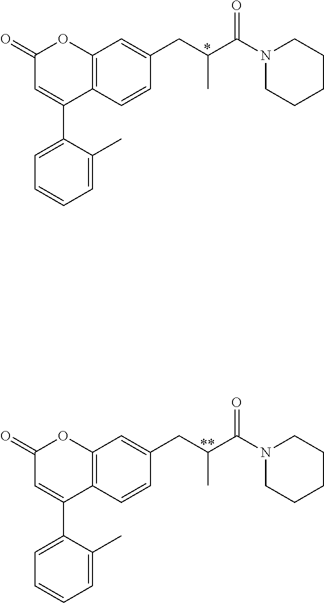 [SMILES: Cc1ccccc1-c1cc(=O)oc2cc(CC(C)C(=O)N3CCCCC3)ccc12 ; Cc1ccccc1-c1cc(=O)oc2cc(CC(C)C(=O)N3CCCCC3)ccc12]

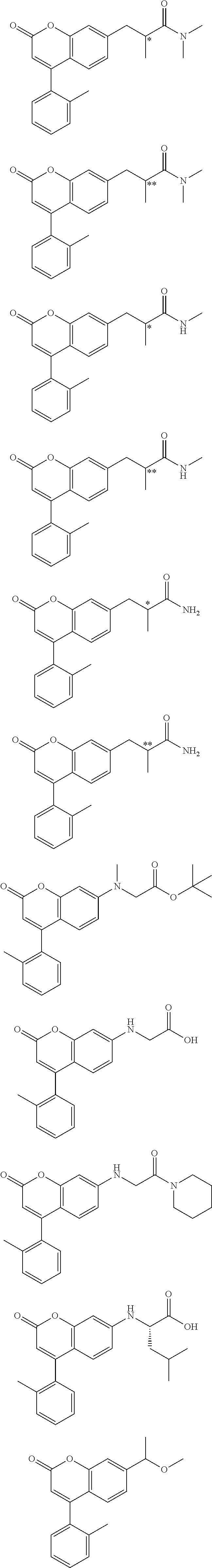

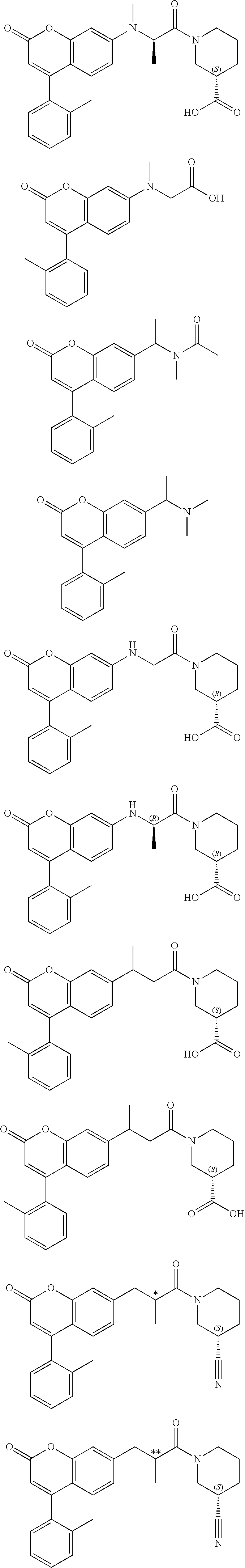

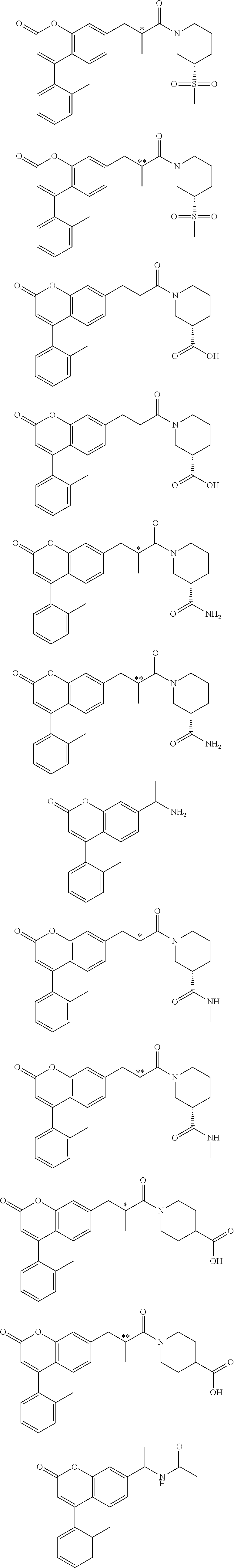

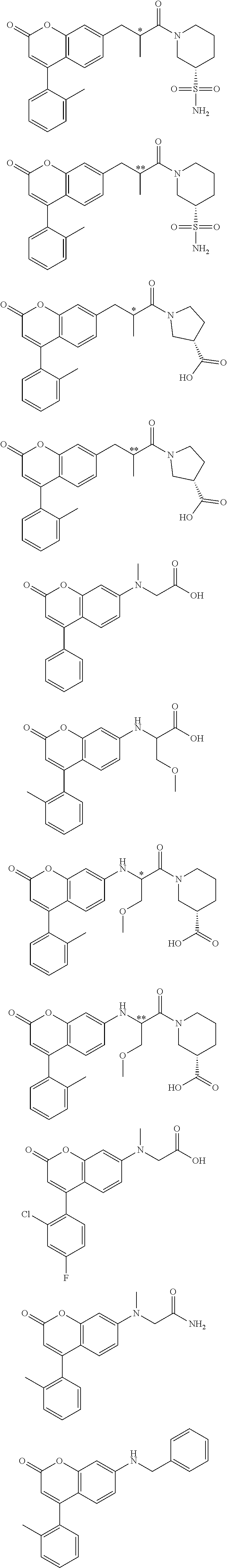

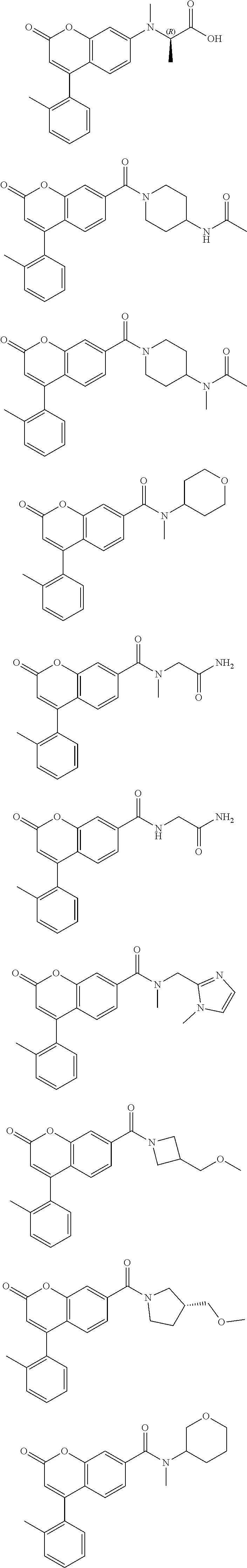

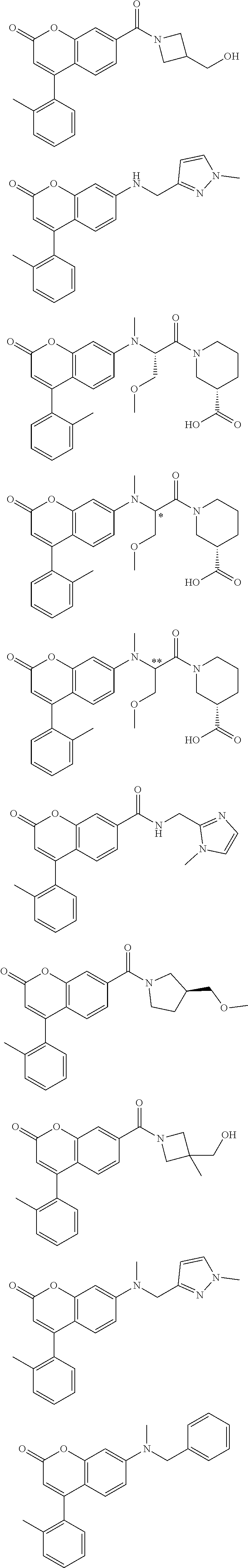

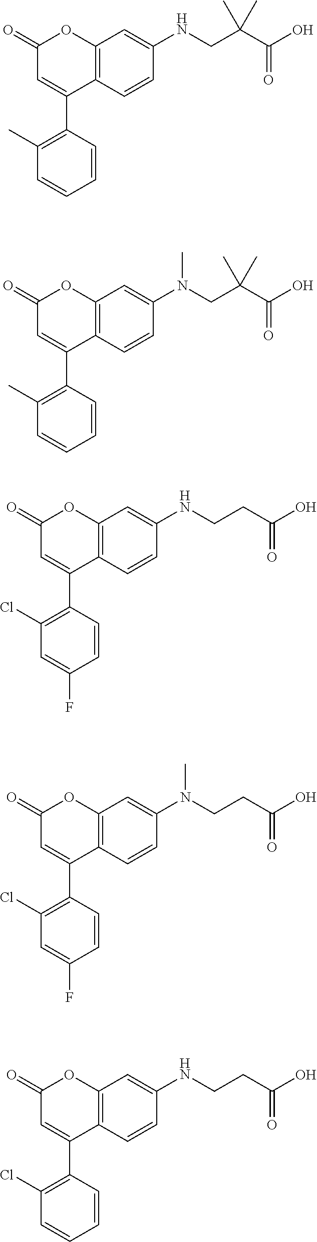 [SMILES: Cc1ccccc1-c1cc(=O)oc2cc(NCC(C)(C)C(=O)O)ccc12 ; Cc1ccccc1-c1cc(=O)oc2cc(N(C)CC(C)(C)C(=O)O)ccc12 ; O=C(O)CCNc1ccc2c(-c3ccc(F)cc3Cl)cc(=O)oc2c1 ; CN(CCC(=O)O)c1ccc2c(-c3ccc(F)cc3Cl)cc(=O)oc2c1 ; O=C(O)CCNc1ccc2c(-c3ccccc3Cl)cc(=O)oc2c1]

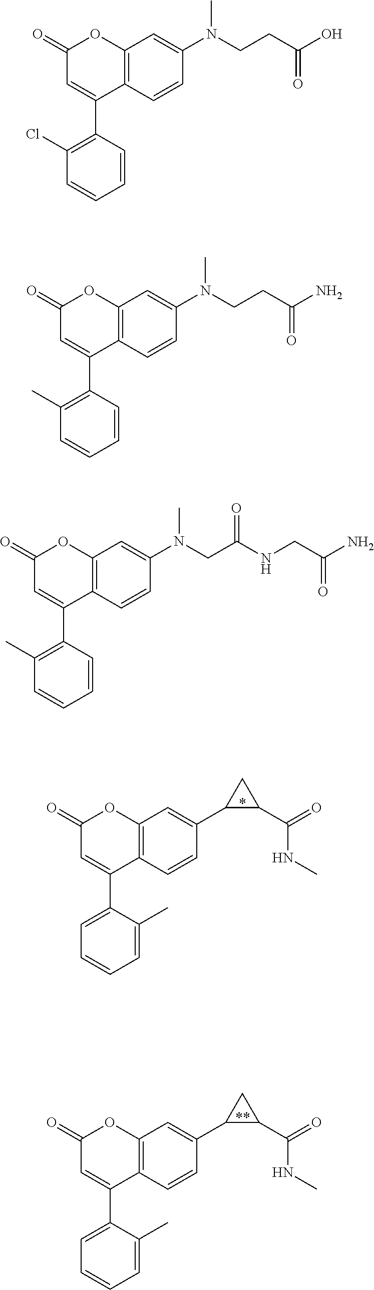 [SMILES: CN(CCC(=O)O)c1ccc2c(-c3ccccc3Cl)cc(=O)oc2c1 ; Cc1ccccc1-c1cc(=O)oc2cc(N(C)CCC(N)=O)ccc12 ; Cc1ccccc1-c1cc(=O)oc2cc(N(C)CC(=O)NCC(N)=O)ccc12 ; CNC(=O)C1CC1c1ccc2c(-c3ccccc3C)cc(=O)oc2c1 ; CNC(=O)C1CC1c1ccc2c(-c3ccccc3C)cc(=O)oc2c1]

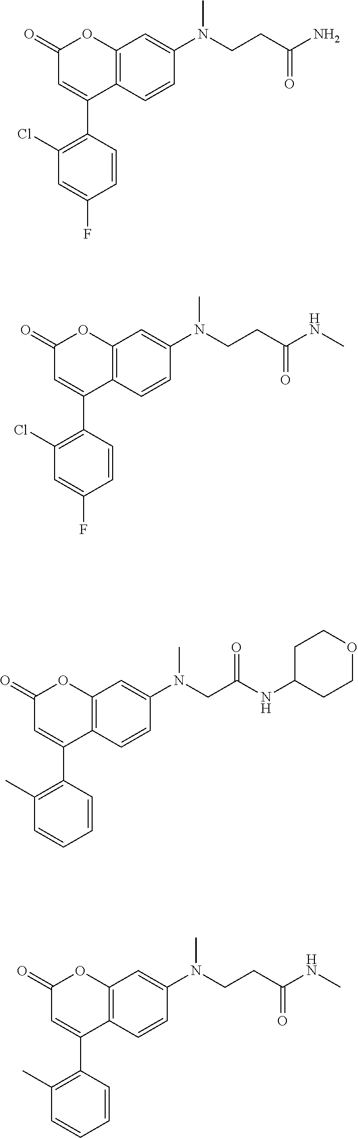 [SMILES: CN(CCC(N)=O)c1ccc2c(-c3ccc(F)cc3Cl)cc(=O)oc2c1 ; CNC(=O)CCN(C)c1ccc2c(-c3ccc(F)cc3Cl)cc(=O)oc2c1 ; Cc1ccccc1-c1cc(=O)oc2cc(N(C)CC(=O)NC3CCOCC3)ccc12 ; CNC(=O)CCN(C)c1ccc2c(-c3ccccc3C)cc(=O)oc2c1]

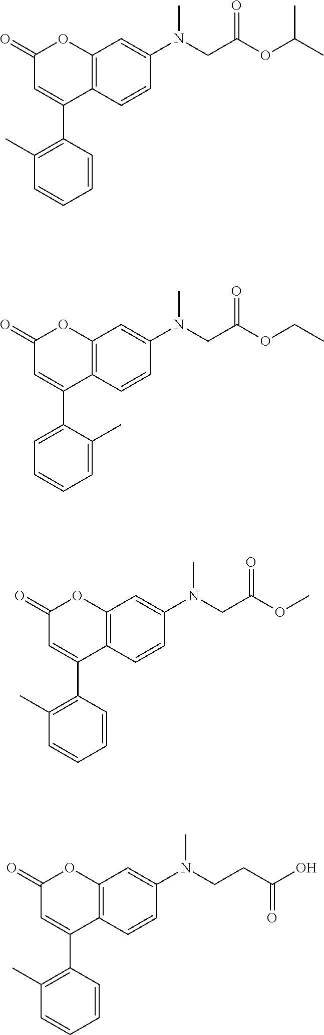 [SMILES: Cc1ccccc1-c1cc(=O)oc2cc(N(C)CC(=O)OC(C)C)ccc12 ; CCOC(=O)CN(C)c1ccc2c(-c3ccccc3C)cc(=O)oc2c1 ; COC(=O)CN(C)c1ccc2c(-c3ccccc3C)cc(=O)oc2c1 ; Cc1ccccc1-c1cc(=O)oc2cc(N(C)CCC(=O)O)ccc12]

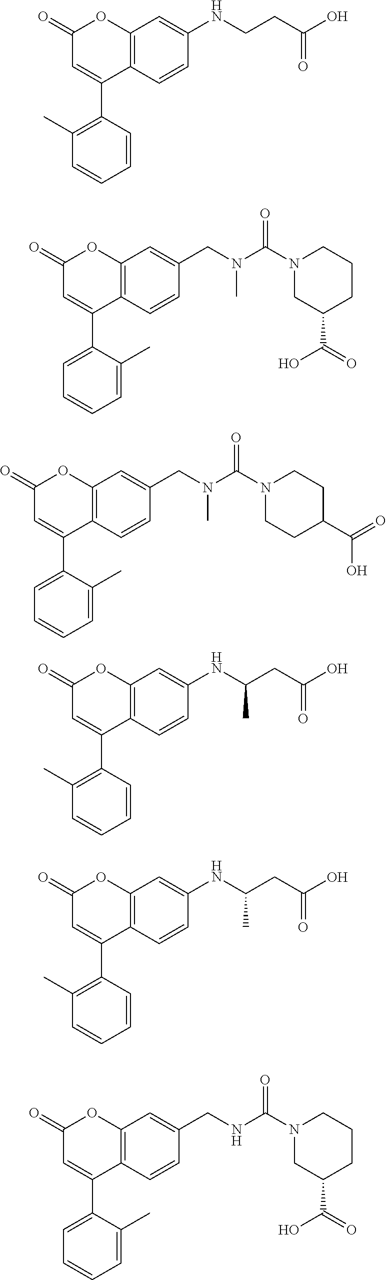

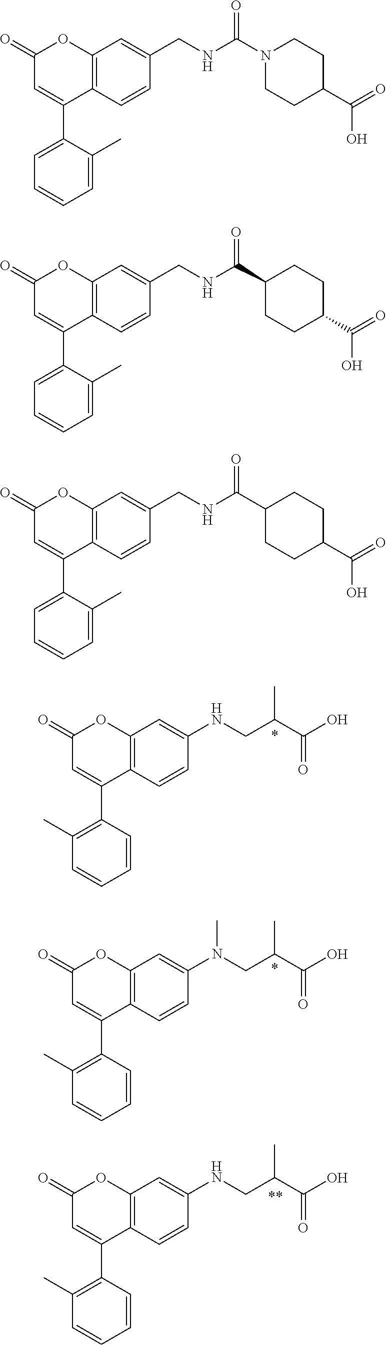 [SMILES: Cc1ccccc1-c1cc(=O)oc2cc(CNC(=O)N3CCC(C(=O)O)CC3)ccc12 ; Cc1ccccc1-c1cc(=O)oc2cc(CNC(=O)[C@H]3CC[C@H](C(=O)O)CC3)ccc12 ; Cc1ccccc1-c1cc(=O)oc2cc(CNC(=O)C3CCC(C(=O)O)CC3)ccc12 ; Cc1ccccc1-c1cc(=O)oc2cc(NCC(C)C(=O)O)ccc12 ; Cc1ccccc1-c1cc(=O)oc2cc(N(C)CC(C)C(=O)O)ccc12 ; Cc1ccccc1-c1cc(=O)oc2cc(NCC(C)C(=O)O)ccc12]

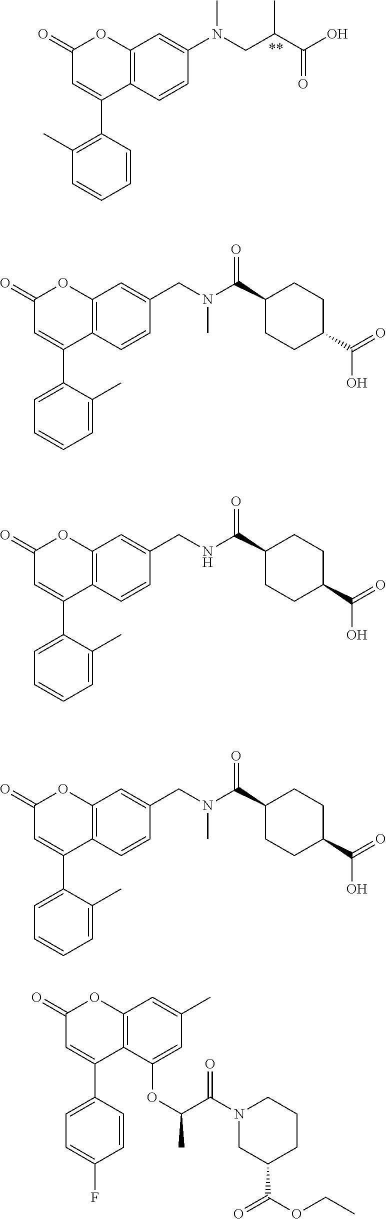 [SMILES: Cc1ccccc1-c1cc(=O)oc2cc(N(C)CC(C)C(=O)O)ccc12 ; Cc1ccccc1-c1cc(=O)oc2cc(CN(C)C(=O)[C@H]3CC[C@H](C(=O)O)CC3)ccc12 ; Cc1ccccc1-c1cc(=O)oc2cc(CNC(=O)[C@H]3CC[C@@H](C(=O)O)CC3)ccc12 ; Cc1ccccc1-c1cc(=O)oc2cc(CN(C)C(=O)[C@H]3CC[C@@H](C(=O)O)CC3)ccc12 ; CCOC(=O)[C@H]1CCCN(C(=O)[C@@H](C)Oc2cc(C)cc3oc(=O)cc(-c4ccc(F)cc4)c23)C1]

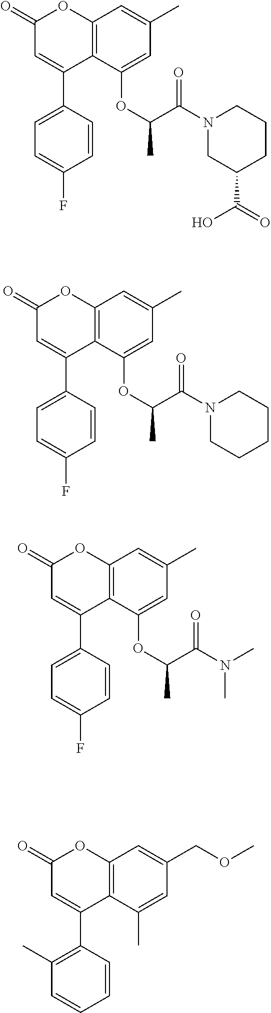 [SMILES: Cc1cc(O[C@H](C)C(=O)N2CCC[C@H](C(=O)O)C2)c2c(-c3ccc(F)cc3)cc(=O)oc2c1 ; Cc1cc(O[C@H](C)C(=O)N2CCCCC2)c2c(-c3ccc(F)cc3)cc(=O)oc2c1 ; Cc1cc(O[C@H](C)C(=O)N(C)C)c2c(-c3ccc(F)cc3)cc(=O)oc2c1 ; COCc1cc(C)c2c(-c3ccccc3C)cc(=O)oc2c1]

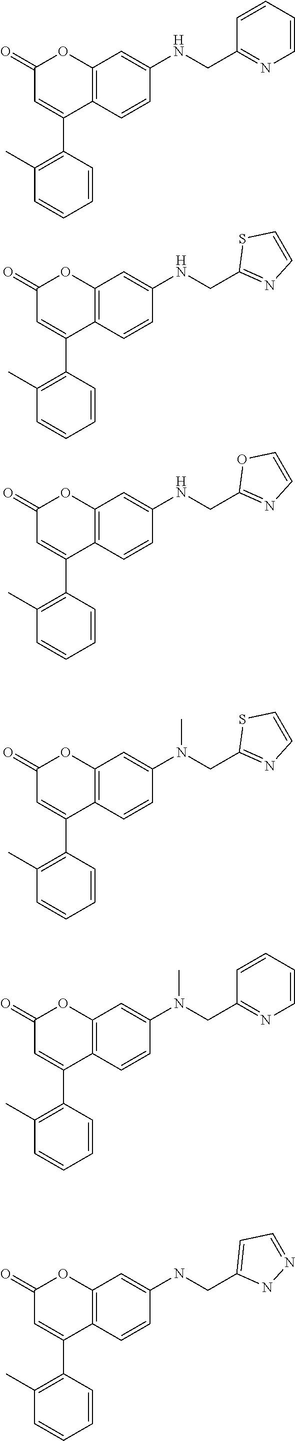

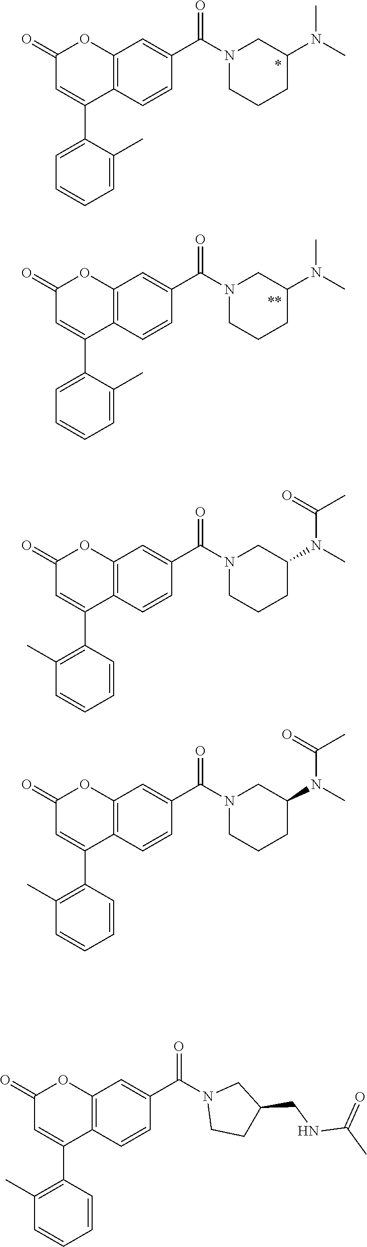 [SMILES: Cc1ccccc1-c1cc(=O)oc2cc(C(=O)N3CCCC(N(C)C)C3)ccc12 ; Cc1ccccc1-c1cc(=O)oc2cc(C(=O)N3CCCC(N(C)C)C3)ccc12 ; CC(=O)N(C)[C@@H]1CCCN(C(=O)c2ccc3c(-c4ccccc4C)cc(=O)oc3c2)C1 ; CC(=O)N(C)[C@H]1CCCN(C(=O)c2ccc3c(-c4ccccc4C)cc(=O)oc3c2)C1 ; CC(=O)NC[C@H]1CCN(C(=O)c2ccc3c(-c4ccccc4C)cc(=O)oc3c2)C1]

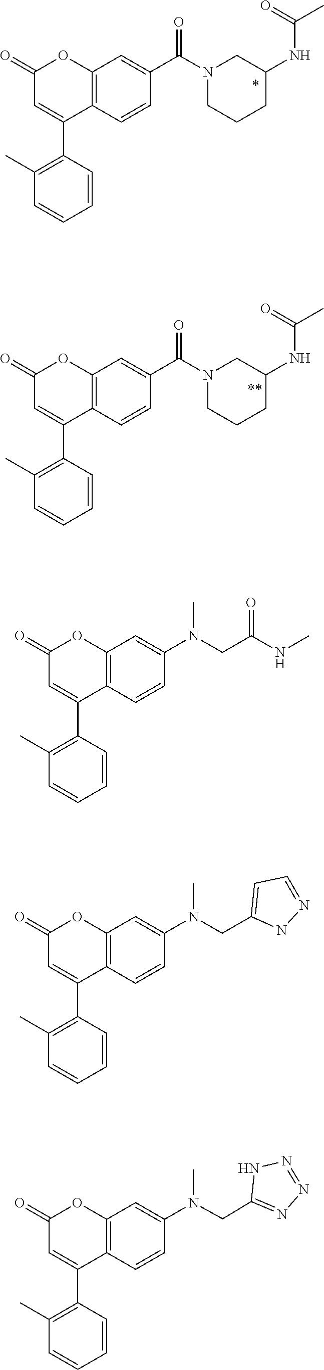

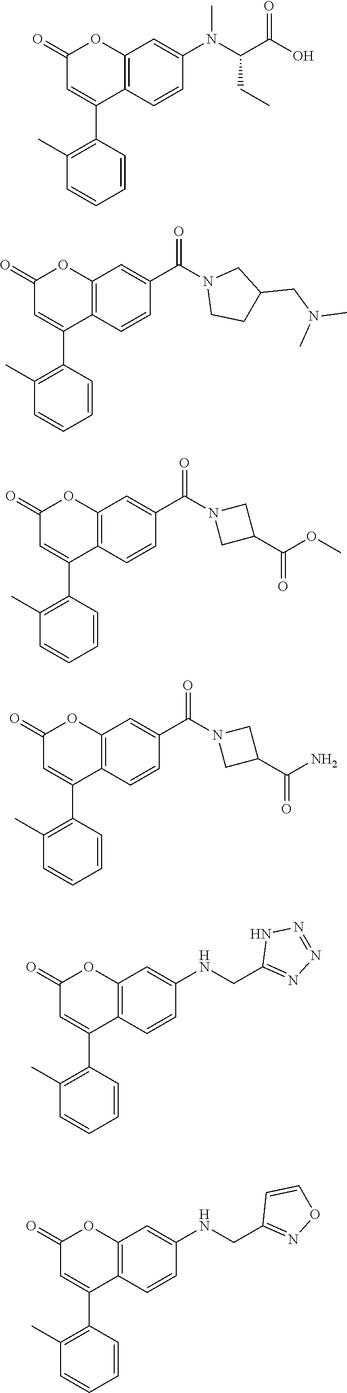 [SMILES: CC[C@@H](C(=O)O)N(C)c1ccc2c(-c3ccccc3C)cc(=O)oc2c1 ; Cc1ccccc1-c1cc(=O)oc2cc(C(=O)N3CCC(CN(C)C)C3)ccc12 ; COC(=O)C1CN(C(=O)c2ccc3c(-c4ccccc4C)cc(=O)oc3c2)C1 ; Cc1ccccc1-c1cc(=O)oc2cc(C(=O)N3CC(C(N)=O)C3)ccc12 ; Cc1ccccc1-c1cc(=O)oc2cc(NCc3nnn[nH]3)ccc12 ; Cc1ccccc1-c1cc(=O)oc2cc(NCc3ccon3)ccc12]

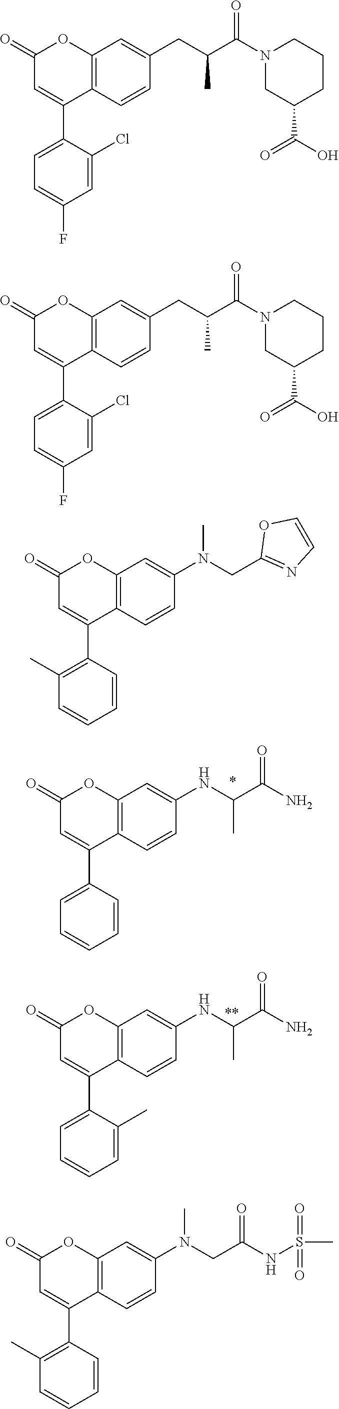

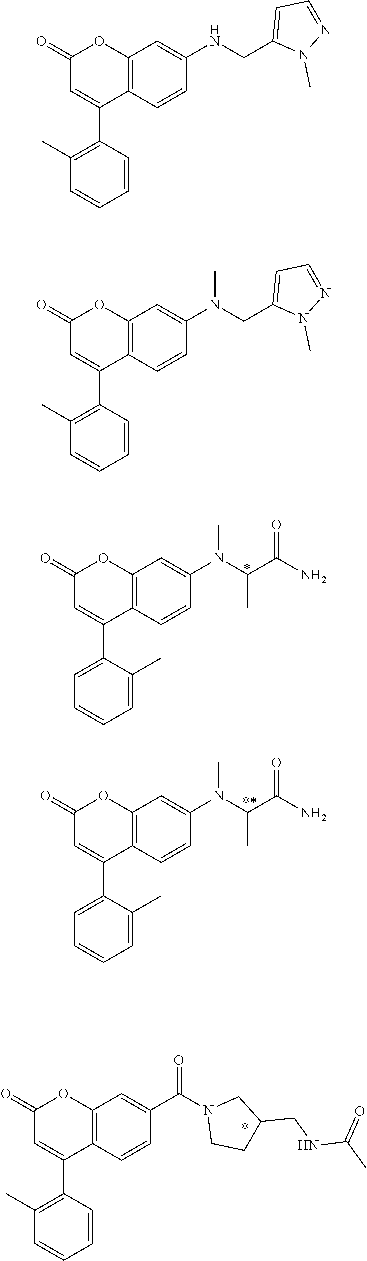 [SMILES: Cc1ccccc1-c1cc(=O)oc2cc(NCc3ccnn3C)ccc12 ; Cc1ccccc1-c1cc(=O)oc2cc(N(C)Cc3ccnn3C)ccc12 ; Cc1ccccc1-c1cc(=O)oc2cc(N(C)C(C)C(N)=O)ccc12 ; Cc1ccccc1-c1cc(=O)oc2cc(N(C)C(C)C(N)=O)ccc12 ; CC(=O)NCC1CCN(C(=O)c2ccc3c(-c4ccccc4C)cc(=O)oc3c2)C1]

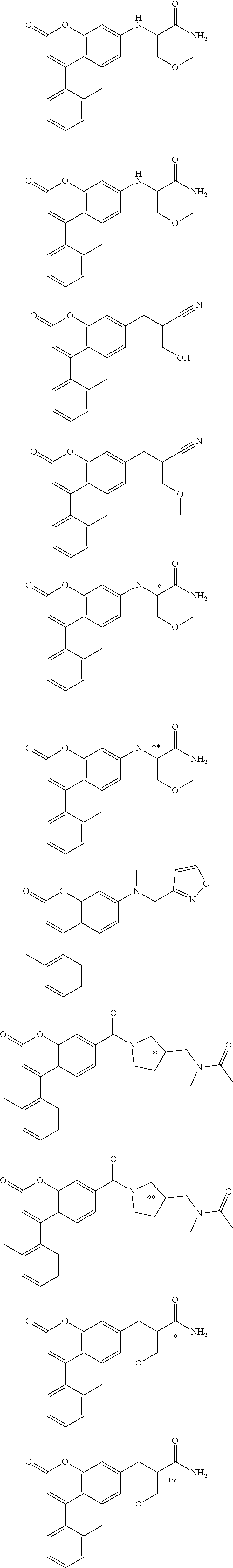

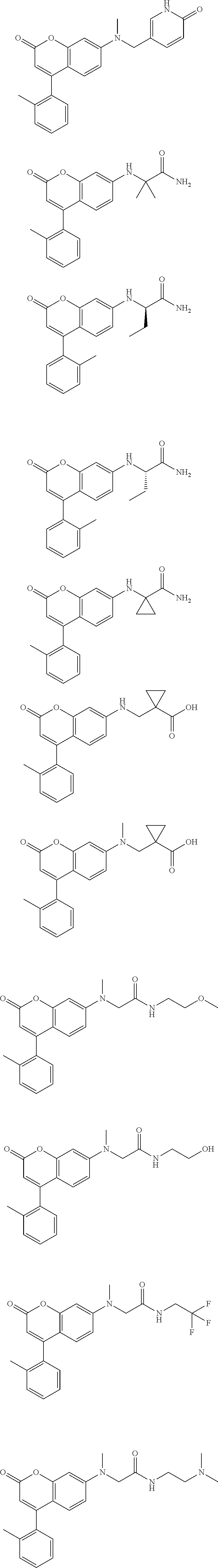

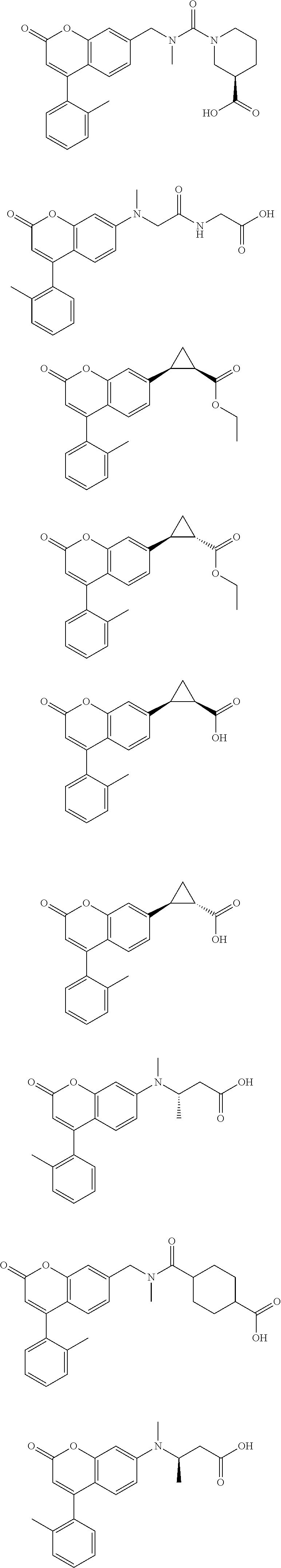

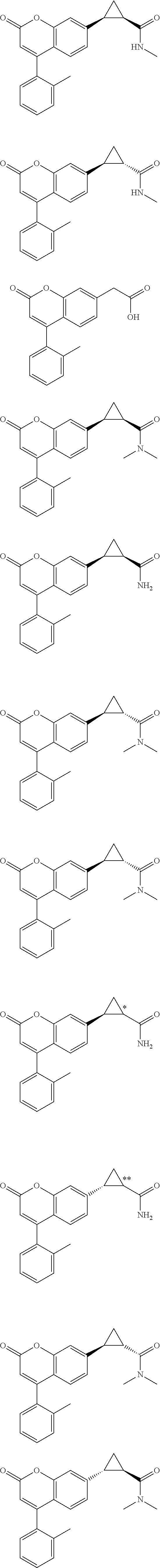

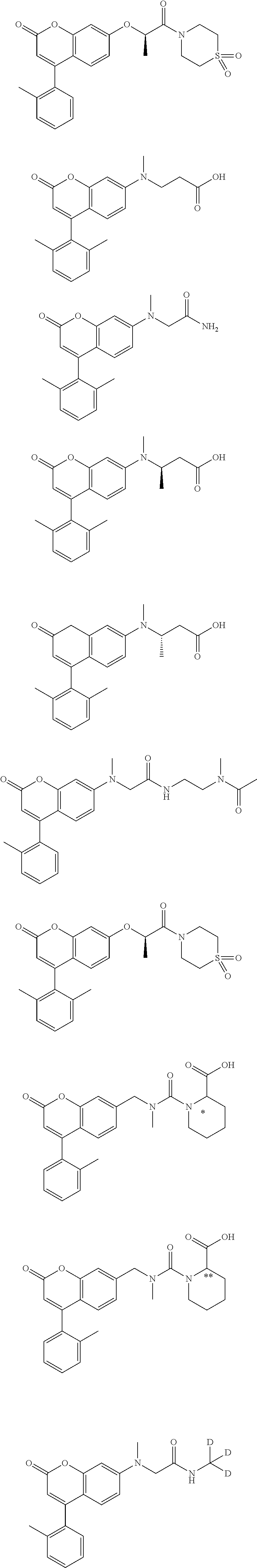

10. The compound according to claim 9, or a pharmaceutically acceptable salt thereof, wherein the compound is selected from the group consisting of:

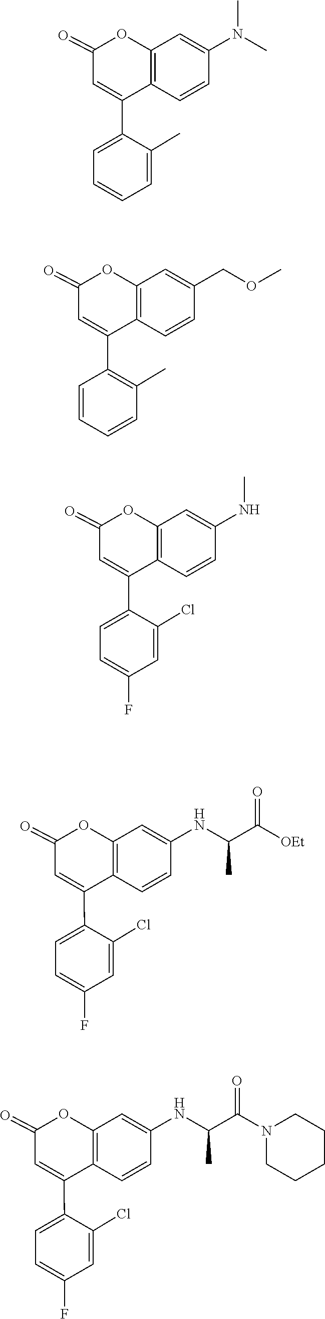 [SMILES: Cc1ccccc1-c1cc(=O)oc2cc(N(C)C)ccc12 ; COCc1ccc2c(-c3ccccc3C)cc(=O)oc2c1 ; CNc1ccc2c(-c3ccc(F)cc3Cl)cc(=O)oc2c1 ; CCOC(=O)[C@@H](C)Nc1ccc2c(-c3ccc(F)cc3Cl)cc(=O)oc2c1 ; C[C@@H](Nc1ccc2c(-c3ccc(F)cc3Cl)cc(=O)oc2c1)C(=O)N1CCCCC1]

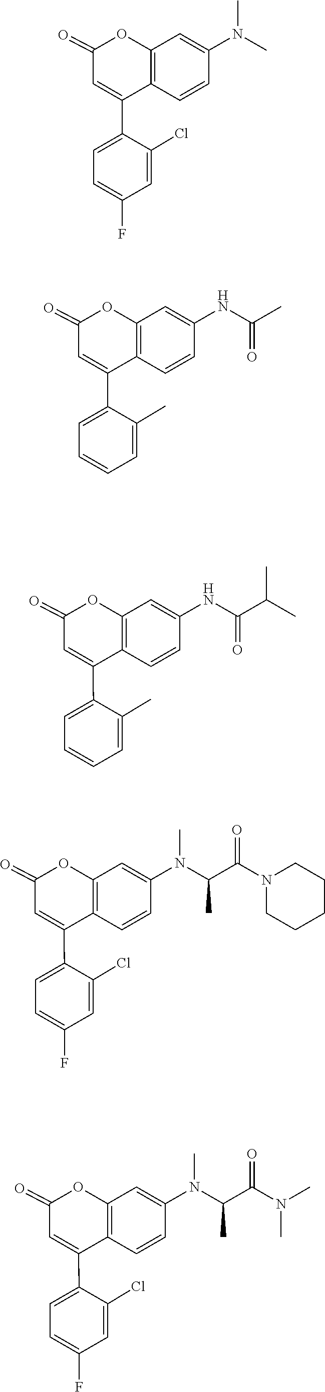 [SMILES: CN(C)c1ccc2c(-c3ccc(F)cc3Cl)cc(=O)oc2c1 ; CC(=O)Nc1ccc2c(-c3ccccc3C)cc(=O)oc2c1 ; Cc1ccccc1-c1cc(=O)oc2cc(NC(=O)C(C)C)ccc12 ; C[C@H](C(=O)N1CCCCC1)N(C)c1ccc2c(-c3ccc(F)cc3Cl)cc(=O)oc2c1 ; C[C@H](C(=O)N(C)C)N(C)c1ccc2c(-c3ccc(F)cc3Cl)cc(=O)oc2c1]

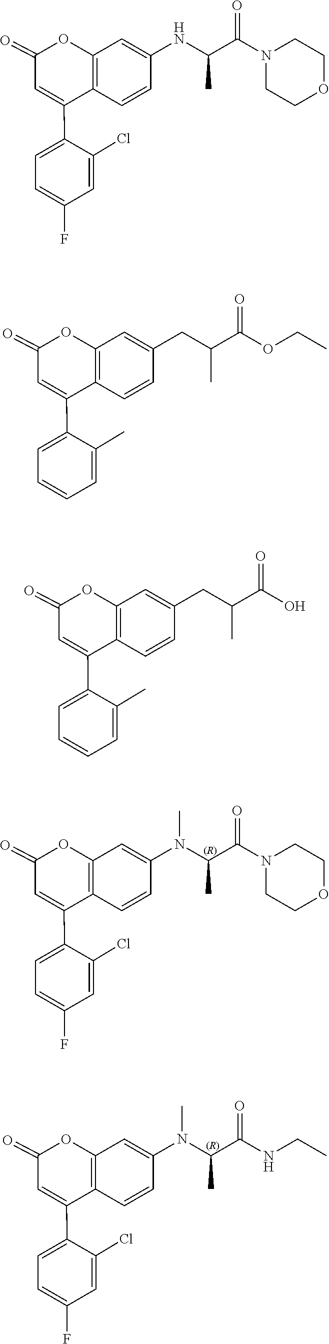

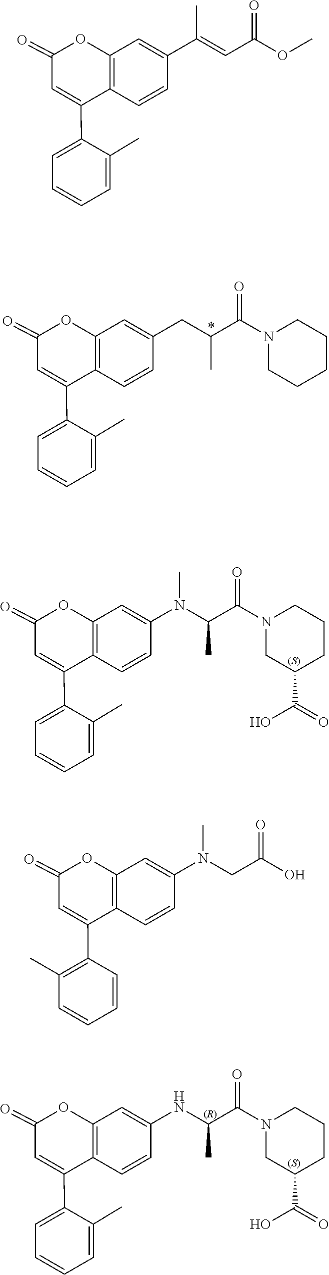

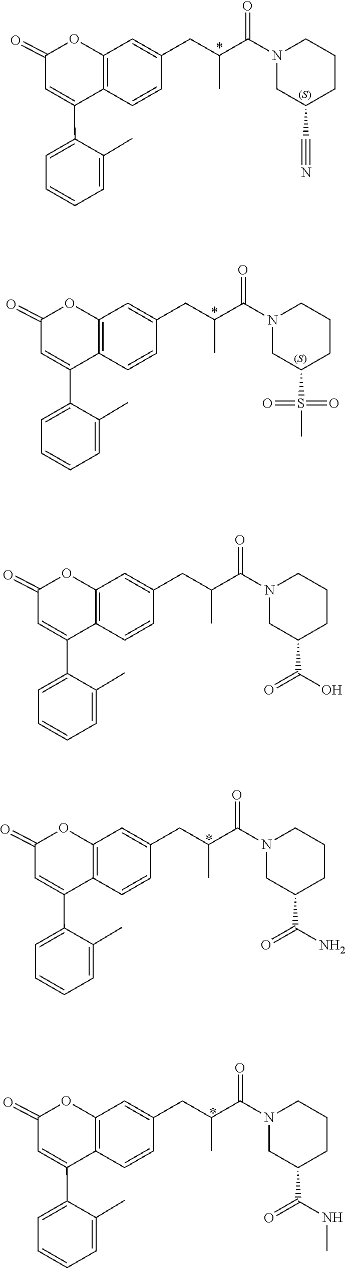

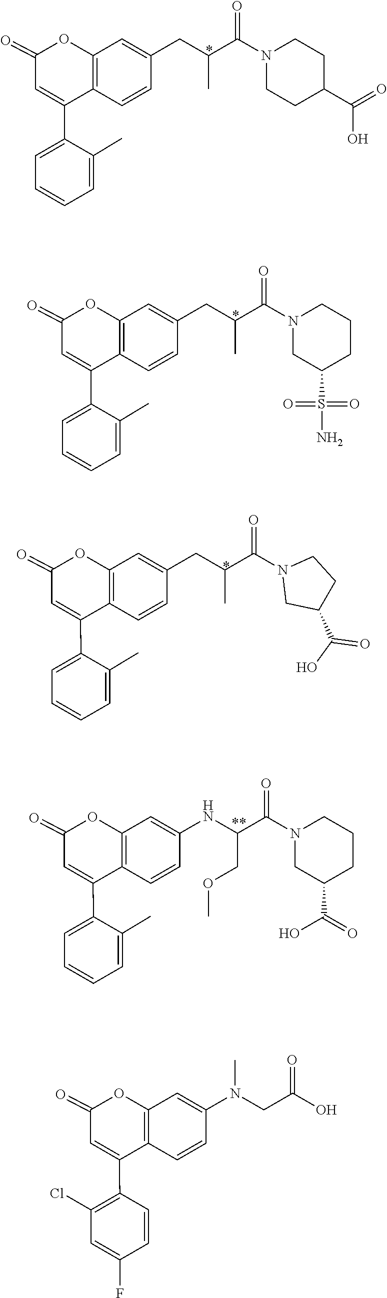 [SMILES: Cc1ccccc1-c1cc(=O)oc2cc(CC(C)C(=O)N3CCC(C(=O)O)CC3)ccc12 ; Cc1ccccc1-c1cc(=O)oc2cc(CC(C)C(=O)N3CCC[C@H](S(N)(=O)=O)C3)ccc12 ; Cc1ccccc1-c1cc(=O)oc2cc(CC(C)C(=O)N3CC[C@H](C(=O)O)C3)ccc12 ; COCC(Nc1ccc2c(-c3ccccc3C)cc(=O)oc2c1)C(=O)N1CCC[C@H](C(=O)O)C1 ; CN(CC(=O)O)c1ccc2c(-c3ccc(F)cc3Cl)cc(=O)oc2c1]

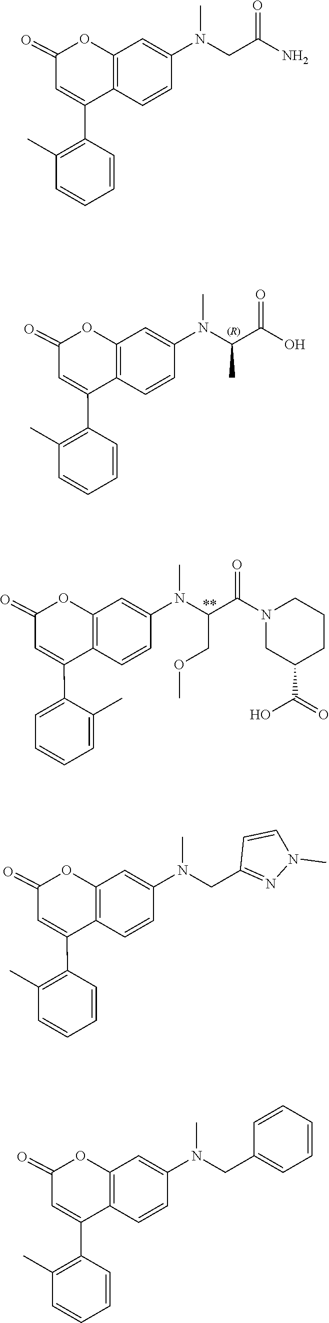

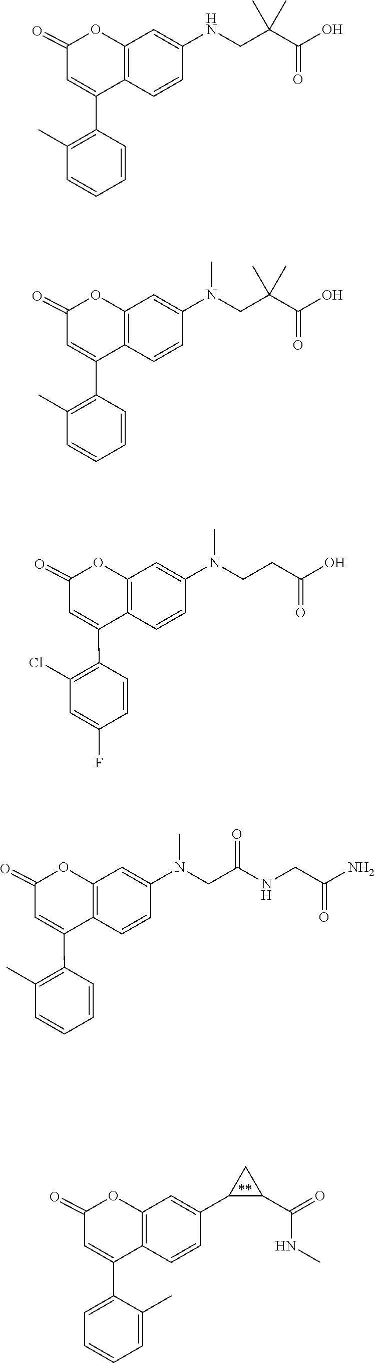 [SMILES: Cc1ccccc1-c1cc(=O)oc2cc(NCC(C)(C)C(=O)O)ccc12 ; Cc1ccccc1-c1cc(=O)oc2cc(N(C)CC(C)(C)C(=O)O)ccc12 ; CN(CCC(=O)O)c1ccc2c(-c3ccc(F)cc3Cl)cc(=O)oc2c1 ; Cc1ccccc1-c1cc(=O)oc2cc(N(C)CC(=O)NCC(N)=O)ccc12 ; CNC(=O)C1CC1c1ccc2c(-c3ccccc3C)cc(=O)oc2c1]

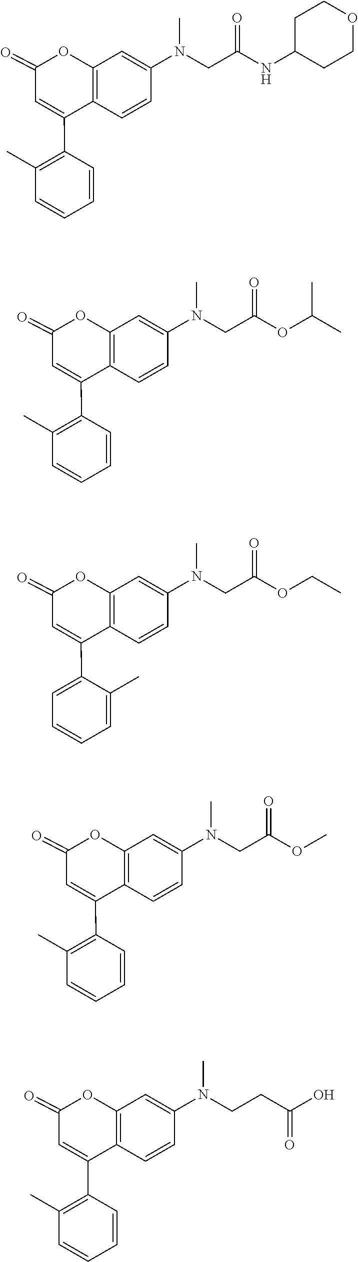 [SMILES: Cc1ccccc1-c1cc(=O)oc2cc(N(C)CC(=O)NC3CCOCC3)ccc12 ; Cc1ccccc1-c1cc(=O)oc2cc(N(C)CC(=O)OC(C)C)ccc12 ; CCOC(=O)CN(C)c1ccc2c(-c3ccccc3C)cc(=O)oc2c1 ; COC(=O)CN(C)c1ccc2c(-c3ccccc3C)cc(=O)oc2c1 ; Cc1ccccc1-c1cc(=O)oc2cc(N(C)CCC(=O)O)ccc12]

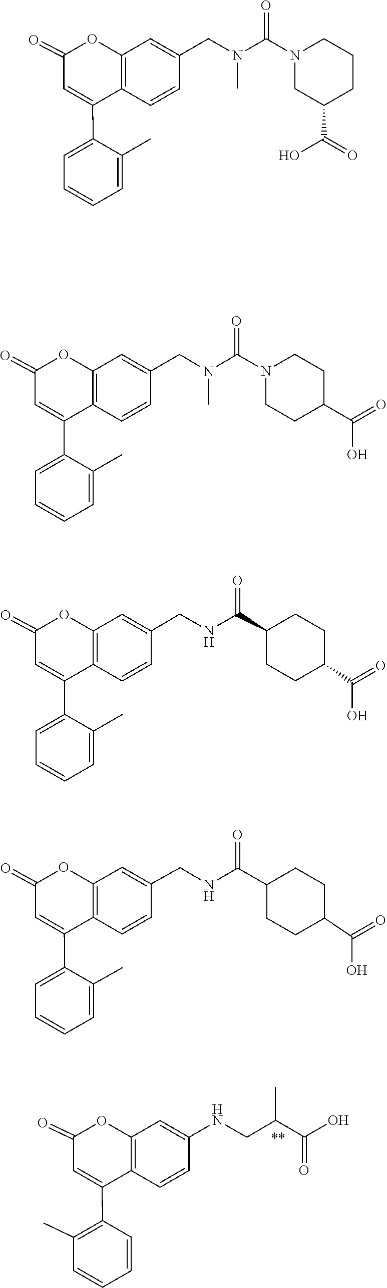 [SMILES: Cc1ccccc1-c1cc(=O)oc2cc(CN(C)C(=O)N3CCC[C@H](C(=O)O)C3)ccc12 ; Cc1ccccc1-c1cc(=O)oc2cc(CN(C)C(=O)N3CCC(C(=O)O)CC3)ccc12 ; Cc1ccccc1-c1cc(=O)oc2cc(CNC(=O)[C@H]3CC[C@H](C(=O)O)CC3)ccc12 ; Cc1ccccc1-c1cc(=O)oc2cc(CNC(=O)C3CCC(C(=O)O)CC3)ccc12 ; Cc1ccccc1-c1cc(=O)oc2cc(NCC(C)C(=O)O)ccc12]

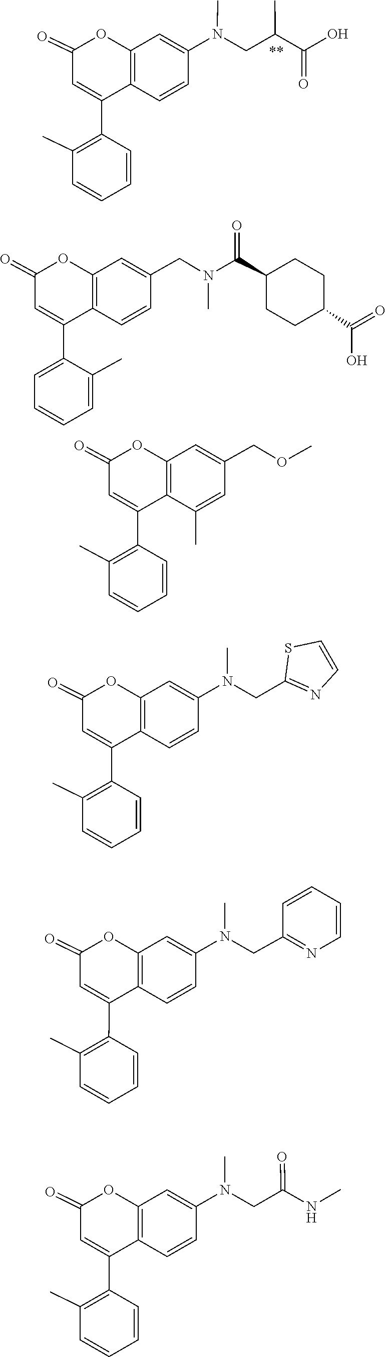

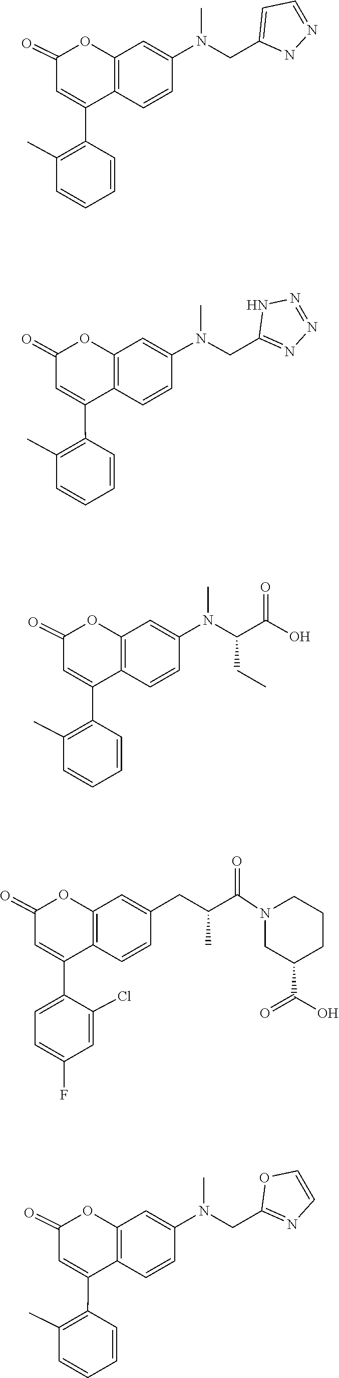

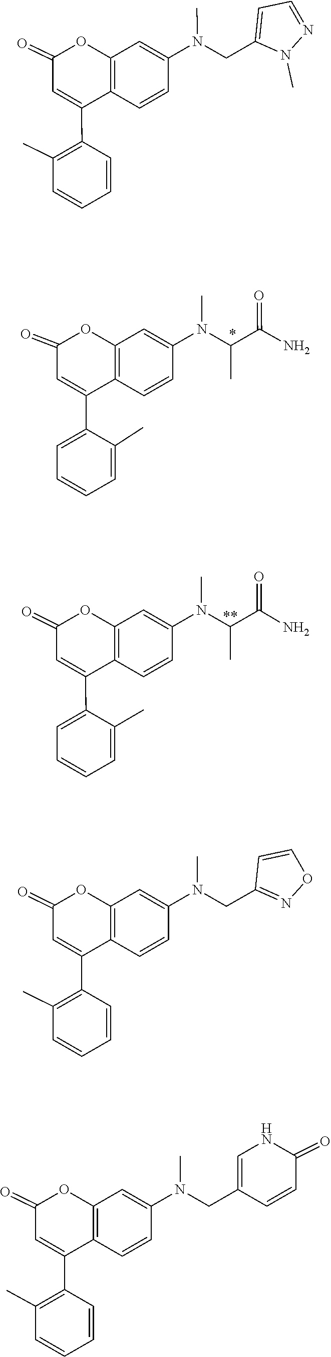 [SMILES: Cc1ccccc1-c1cc(=O)oc2cc(N(C)Cc3ccnn3C)ccc12 ; Cc1ccccc1-c1cc(=O)oc2cc(N(C)C(C)C(N)=O)ccc12 ; Cc1ccccc1-c1cc(=O)oc2cc(N(C)C(C)C(N)=O)ccc12 ; Cc1ccccc1-c1cc(=O)oc2cc(N(C)Cc3ccon3)ccc12 ; Cc1ccccc1-c1cc(=O)oc2cc(N(C)Cc3ccc(=O)[nH]c3)ccc12]

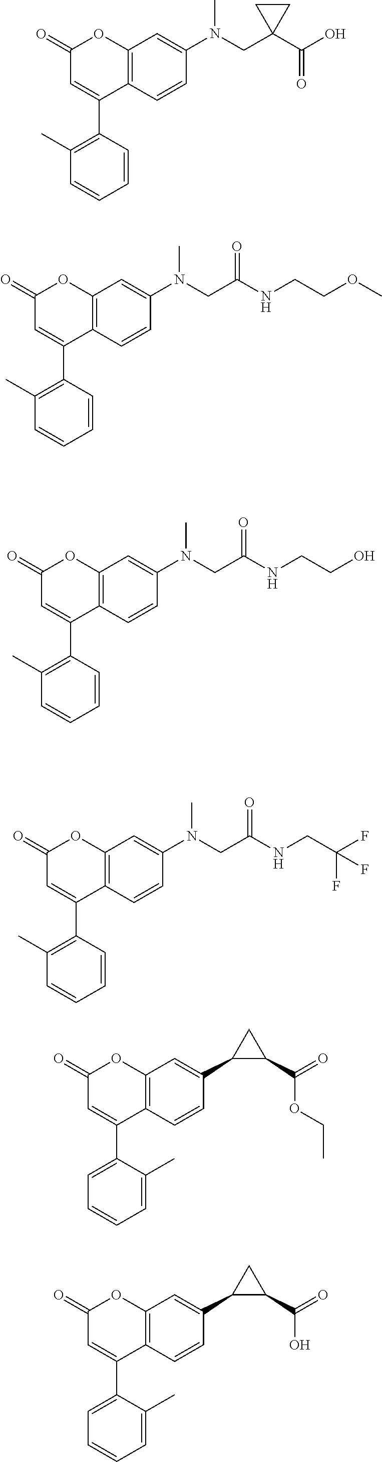 [SMILES: Cc1ccccc1-c1cc(=O)oc2cc(N(C)CC3(C(=O)O)CC3)ccc12 ; COCCNC(=O)CN(C)c1ccc2c(-c3ccccc3C)cc(=O)oc2c1 ; Cc1ccccc1-c1cc(=O)oc2cc(N(C)CC(=O)NCCO)ccc12 ; Cc1ccccc1-c1cc(=O)oc2cc(N(C)CC(=O)NCC(F)(F)F)ccc12 ; CCOC(=O)[C@@H]1C[C@@H]1c1ccc2c(-c3ccccc3C)cc(=O)oc2c1 ; Cc1ccccc1-c1cc(=O)oc2cc([C@H]3C[C@H]3C(=O)O)ccc12]

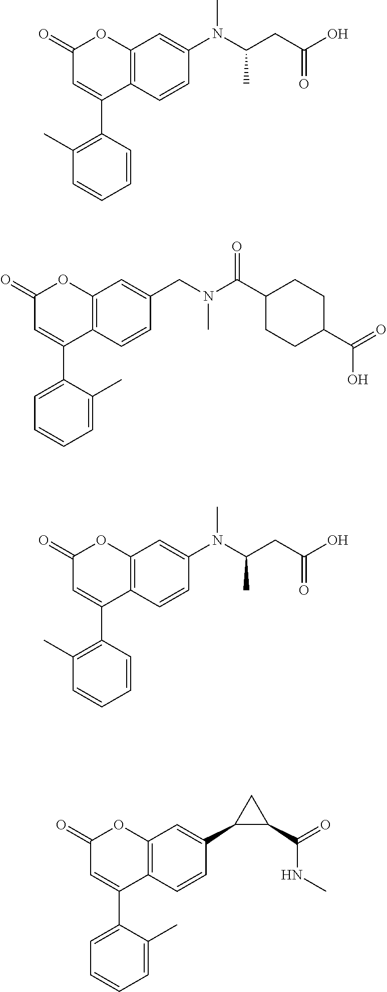 [SMILES: Cc1ccccc1-c1cc(=O)oc2cc(N(C)[C@@H](C)CC(=O)O)ccc12 ; Cc1ccccc1-c1cc(=O)oc2cc(CN(C)C(=O)C3CCC(C(=O)O)CC3)ccc12 ; Cc1ccccc1-c1cc(=O)oc2cc(N(C)[C@H](C)CC(=O)O)ccc12 ; CNC(=O)[C@@H]1C[C@@H]1c1ccc2c(-c3ccccc3C)cc(=O)oc2c1]

11. The compound according to claim 10, or a pharmaceutically acceptable salt thereof, wherein the compound is selected from the group consisting of:

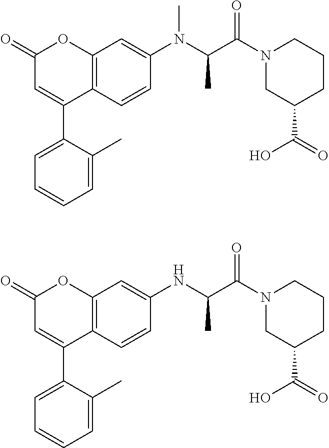 [SMILES: Cc1ccccc1-c1cc(=O)oc2cc(N(C)[C@H](C)C(=O)N3CCC[C@H](C(=O)O)C3)ccc12 ; Cc1ccccc1-c1cc(=O)oc2cc(N[C@H](C)C(=O)N3CCC[C@H](C(=O)O)C3)ccc12]

12. The compound according to claim 10, or a pharmaceutically acceptable salt thereof, wherein the compound is selected from the group consisting of:

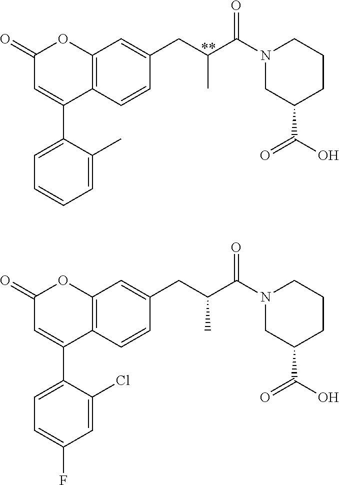 [SMILES: Cc1ccccc1-c1cc(=O)oc2cc(CC(C)C(=O)N3CCC[C@H](C(=O)O)C3)ccc12 ; C[C@H](Cc1ccc2c(-c3ccc(F)cc3Cl)cc(=O)oc2c1)C(=O)N1CCC[C@H](C(=O)O)C1]

13. The compound according to claim 10, or a pharmaceutically acceptable salt thereof, wherein the compound is selected from the group consisting of:

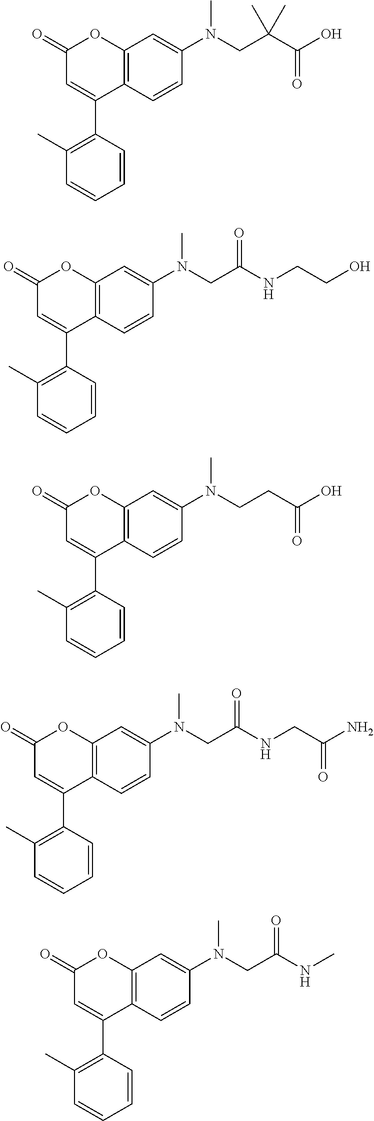 [SMILES: Cc1ccccc1-c1cc(=O)oc2cc(N(C)CC(C)(C)C(=O)O)ccc12 ; Cc1ccccc1-c1cc(=O)oc2cc(N(C)CC(=O)NCCO)ccc12 ; Cc1ccccc1-c1cc(=O)oc2cc(N(C)CCC(=O)O)ccc12 ; Cc1ccccc1-c1cc(=O)oc2cc(N(C)CC(=O)NCC(N)=O)ccc12 ; CNC(=O)CN(C)c1ccc2c(-c3ccccc3C)cc(=O)oc2c1]

14. The compound according to claim 10, or a pharmaceutically acceptable salt thereof, wherein the compound is:

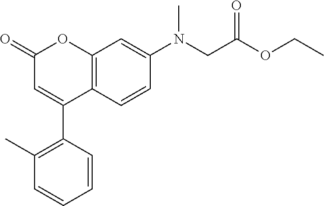 [SMILES: CCOC(=O)CN(C)c1ccc2c(-c3ccccc3C)cc(=O)oc2c1]

15. A pharmaceutical composition comprising a compound according to claim 10, and a pharmaceutically acceptable excipient.

16. A method of inhibiting the activity of POLRMT with a compound of formula (I), or a pharmaceutically acceptable salt thereof: 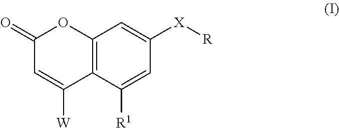
wherein;
X is C(R³)(R³), C₃-C₆ cycloalkyl, C(R³)(R³)O, C(R³)(R³)OC(O), C(R³)═C(R³), N(R⁴), C(R³)(R³)N(R³), C(O)(NR³), or C(O);
W is C₆-C₁₂ aryl or 5-12 membered heteroaryl, either of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium;
R is hydrogen, NR³R⁴, C(O)NR³R⁴, C₁-C₄ alkylcarboxyl, C(O)R³, carboxyl, CH₂CH₂R², hydroxyl, or C₁-C₆ alkoxyl optionally substituted with one or more groups, each independently selected from the group consisting of hydroxyl, fluoro, C₁-C₄ alkoxyl, NR³R⁴, C₁-C₄ alkylcarboxyl, carboxyl, and C(O)NR³R⁴,
or R is NR³C(O)R³, C₃-C₆ cycloalkyl optionally substituted with C(O)NR³R³, or C₄-C₆ heterocyclic,
or R is C₁-C₆ alkyl optionally substituted with one or more groups, each independently selected from the group consisting of hydroxyl, fluoro, cyano, C₁-C₄ alkoxyl, C₁-C₄ haloalkoxyl, phenyl, NR³R⁴, C₁-C₄ alkylcarboxyl, carboxyl, CR⁵R⁶—CO₂R⁷, C(O)NR³R⁴, C(O)NR³SO₂R³, NR³C(O)R³, C₃-C₆ cycloalkyl, 5- to 6-membered heterocyclic ring optionally substituted with R³, and heteroaryl optionally substituted with C₁-C₄ alkyl, hydroxyl, or oxo,
or R is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, and C₁-C₄ alkoxyl;
R¹ is hydrogen, hydroxyl, cyano, C₁-C₃ alkyl optionally substituted with one or more fluoro, C₃-C₄ cycloalkyl, or methoxyl optionally substituted with one to three fluoro;
R² is C₆-C₁₀ aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R² is C₁-C₄ haloalkoxyl, OR³, NR³R⁴, C(O)R³, or C(O)NR³R⁴;
each R³ is independently hydrogen, or C₃-C₆ cycloalkyl, C₄-C₆ heterocyclic, C₁-C₄ alkyl, each optionally substituted with one or more fluoro or carboxyl;
each R⁴ is independently R³, C(O) C₁-C₄ alkyl optionally substituted with one or more fluoro groups, C(O)NHC₁-C₄ alkyl optionally substituted with one or more fluoro groups, or C₁-C₄ alkyl optionally substituted with one or more deuterium, hydroxyl, fluoro, C₁-C₃ alkoxyl, NR³R³, carboxyl, or C(O)NR³R³,
or if R³ and R⁴ are attached to the same nitrogen atom, R³ and R⁴ together with their connecting nitrogen form a 4- to 6-membered heterocyclic ring optionally containing another heteroatom that is N, O, or S, and such heterocyclic ring is optionally substituted with one or more groups each independently selected from the group consisting of fluoro, chloro, cyano, carboxyl, C(O)NR³R³, NR³R⁴, NR¹C(O)R³, S(O)₂R³, S(O)₂NR³R³, C₁-C₄ alkylcarboxylate, and C₁-C₄ alkyl optionally substituted with hydroxyl, C₁-C₄ alkoxy, or NR¹C(O)R³;
R⁵ and R⁶ together with their connecting carbon form a 3- to 5-membered cyclic or heterocyclic ring; and
R⁷ is H or NH₂.

17. The method according to claim 16, wherein;
X is C(R³)(R³), C₃-C₆ cycloalkyl, C(R³)(R³)O, C(R³)(R³)OC(O), C(R³)═C(R³), N(R⁴), C(R³)(R³)N(R³), C(O)(NR³), or C(O);
W is C₆ aryl optionally substituted with one or more groups, each independently selected from the group consisting of fluoro, chloro, C₁ alkoxyl, and C₁ alkyl optionally substituted with one or more deuterium;
R is hydrogen, NR³R⁴, C(O)NR³R⁴, C₁-C₂ alkylcarboxyl, C(O)R³, carboxyl, hydroxyl, C₁ alkoxyl, NR³C(O)R³, C₆ aryl, or C₃-C₈ cycloalkyl optionally substituted with C(O)NR³R³,
or R is C₁-C₈alkyl optionally substituted with one or more groups, each independently selected from the group consisting of hydroxyl, cyano, C₁ alkoxyl, C₁ haloalkoxyl, phenyl, NR³R⁴, C₁-C₄ alkylcarboxyl, carboxyl, CR⁵R⁶—CO₂R⁷, C(O)NR³R⁴, C(O)NR³SO₂R³, NR³C(O)R³, C₃ cycloalkyl, 5- to 6-membered heterocyclic ring optionally substituted with R³, and heteroaryl optionally substituted with C₁-C₄ alkyl, hydroxyl, or oxo;
R¹ is hydrogen, hydroxyl, or C₁ alkyl;
each R³ is independently hydrogen, or C₆ cycloalkyl, C₆ heterocyclic, C₁-C₃ alkyl, each optionally substituted with carboxyl;
each R⁴ is independently R³, or C₁-C₃ alkyl optionally substituted with one or more deuterium, hydroxyl, fluoro, C₁ alkoxyl, NR³R³, carboxyl, or C(O)NR³R³;
or if R³ and R⁴ are attached to the same nitrogen atom, R³ and R⁴ together with their connecting nitrogen form a 4- to 6-membered heterocyclic ring optionally containing another heteroatom that is O, and such heterocyclic ring is optionally substituted with one or more groups each independently selected from the group consisting of cyano, carboxyl, C(O)NR³R³, NR³R⁴, NR¹C(O)R³, S(O)₂R³, S(O)₂NR³R³, C₁-C₂ alkylcarboxylate, and C₁ alkyl optionally substituted with hydroxyl, C₁ alkoxy, or NR¹C(O)R³;
R⁵ and R⁶ together with their connecting carbon form a 3- to 5-membered cyclic or heterocyclic ring; and
R⁷ is H or NH₂.

18. The method according to claim 16, wherein;
X is C(R³)(R³), C₃ cycloalkyl, C(R³)═C(R³), N(R⁴), or C(R³)(R³)N(R³);
W is C₆ aryl optionally substituted with one or more groups, each independently selected from the group consisting of fluoro, chloro, and C₁ alkyl;
R is C(O)NR³R⁴, C₁-C₂ alkylcarboxyl, C(O)R³, carboxyl, or C₁ alkoxyl,
or R is C₁-C₄ alkyl optionally substituted with one or more groups, each independently selected from the group consisting of C₁ alkoxyl, phenyl, C₁-C₄ alkylcarboxyl, carboxyl, C(O)NR³R⁴, C₃ cycloalkyl, 5-membered heterocyclic, and heteroaryl optionally substituted with C₁ alkyl, hydroxyl, or oxo;
each R³ is independently hydrogen, or C₆ cycloalkyl, C₆ heterocyclic, C₁-C₃ alkyl, each optionally substituted with carboxyl;
each R⁴ is independently R³, C₁-C₂ alkyl optionally substituted with one or more hydroxyl, fluoro, C₁ alkoxyl, or C(O)NR³R³;
or if R³ and R⁴ are attached to the same nitrogen atom, R³ and R⁴ together with their connecting nitrogen form a 5- to 6-membered heterocyclic ring optionally containing another heteroatom that is O, and such heterocyclic ring is optionally substituted with one or more groups each independently selected from the group consisting of cyano, carboxyl, C(O)NR³R³, S(O)₂R³, and S(O)₂NR³R³.

19. The method according to claim 16, wherein the compound is selected from the group consisting of: 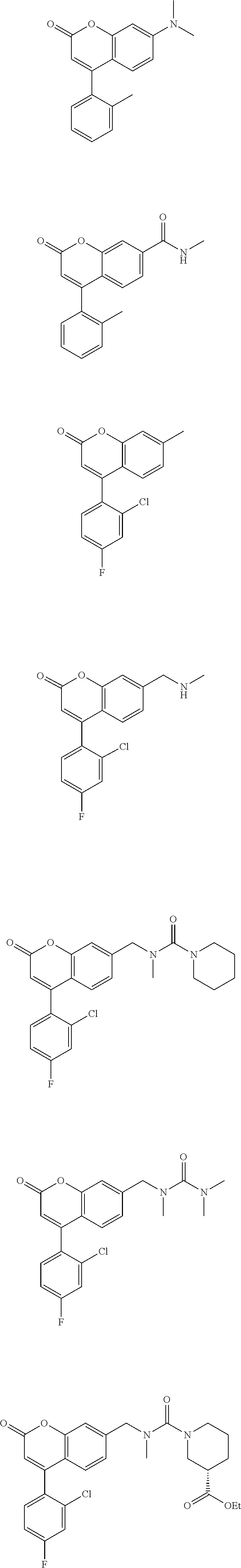 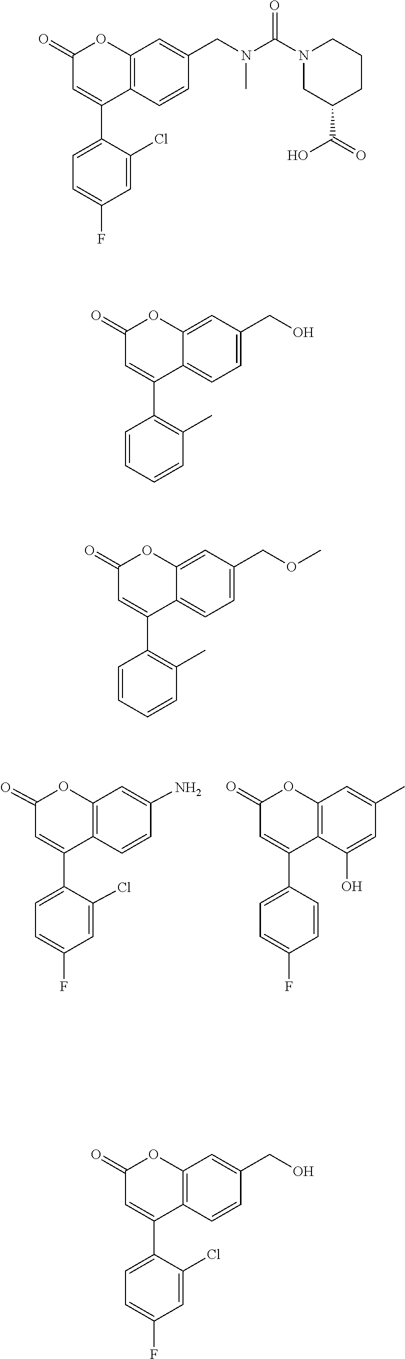 [SMILES: CN(Cc1ccc2c(-c3ccc(F)cc3Cl)cc(=O)oc2c1)C(=O)N1CCC[C@H](C(=O)O)C1 ; Cc1ccccc1-c1cc(=O)oc2cc(CO)ccc12 ; COCc1ccc2c(-c3ccccc3C)cc(=O)oc2c1 ; Nc1ccc2c(-c3ccc(F)cc3Cl)cc(=O)oc2c1 ; Cc1cc(O)c2c(-c3ccc(F)cc3)cc(=O)oc2c1 ; O=c1cc(-c2ccc(F)cc2Cl)c2ccc(CO)cc2o1] 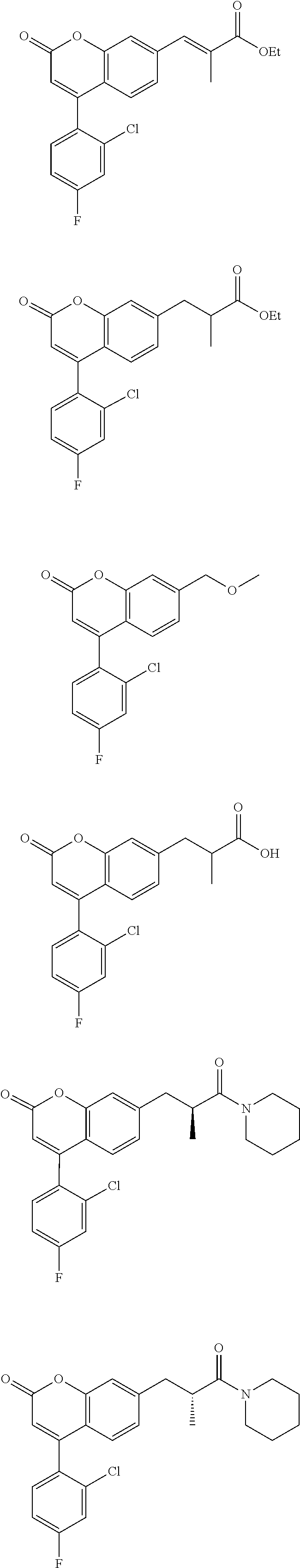 [SMILES: CCOC(=O)/C(C)=C/c1ccc2c(-c3ccc(F)cc3Cl)cc(=O)oc2c1 ; CCOC(=O)C(C)Cc1ccc2c(-c3ccc(F)cc3Cl)cc(=O)oc2c1 ; COCc1ccc2c(-c3ccc(F)cc3Cl)cc(=O)oc2c1 ; CC(Cc1ccc2c(-c3ccc(F)cc3Cl)cc(=O)oc2c1)C(=O)O ; C[C@@H](Cc1ccc2c(-c3ccc(F)cc3Cl)cc(=O)oc2c1)C(=O)N1CCCCC1 ; C[C@H](Cc1ccc2c(-c3ccc(F)cc3Cl)cc(=O)oc2c1)C(=O)N1CCCCC1] 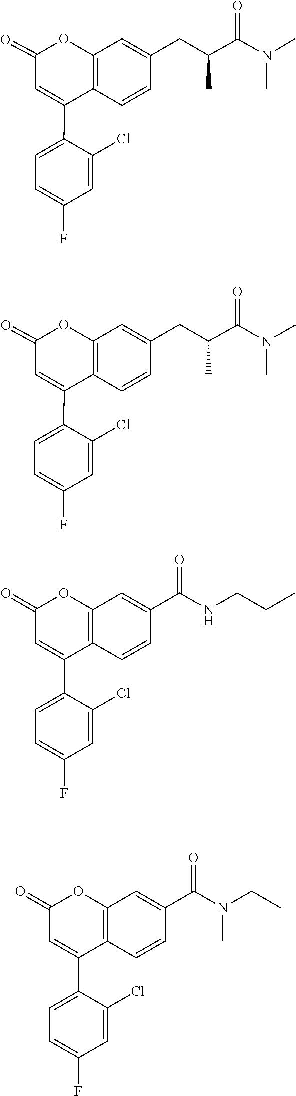 [SMILES: C[C@@H](Cc1ccc2c(-c3ccc(F)cc3Cl)cc(=O)oc2c1)C(=O)N(C)C ; C[C@H](Cc1ccc2c(-c3ccc(F)cc3Cl)cc(=O)oc2c1)C(=O)N(C)C ; CCCNC(=O)c1ccc2c(-c3ccc(F)cc3Cl)cc(=O)oc2c1 ; CCN(C)C(=O)c1ccc2c(-c3ccc(F)cc3Cl)cc(=O)oc2c1] 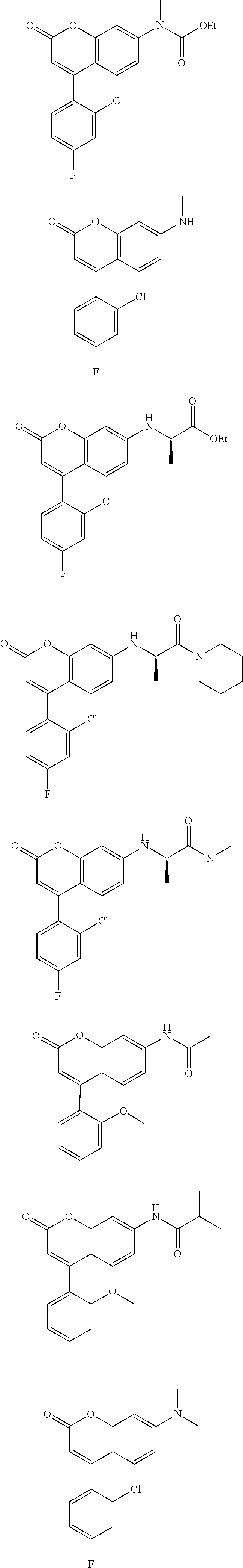 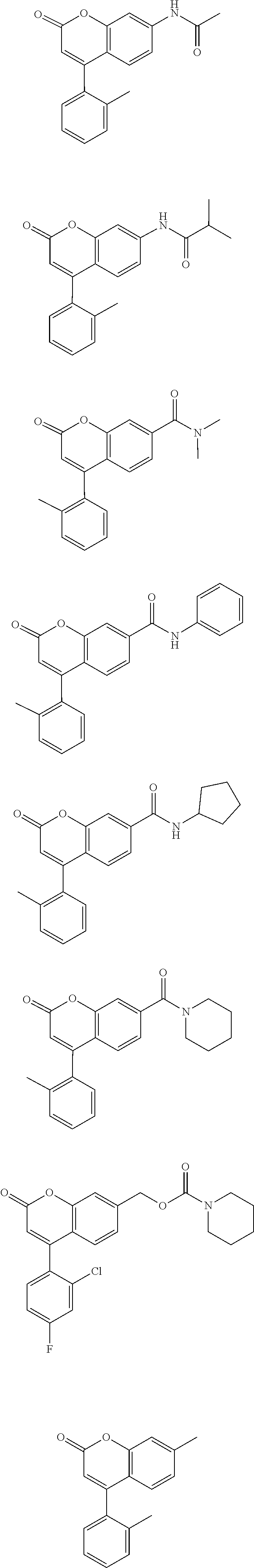 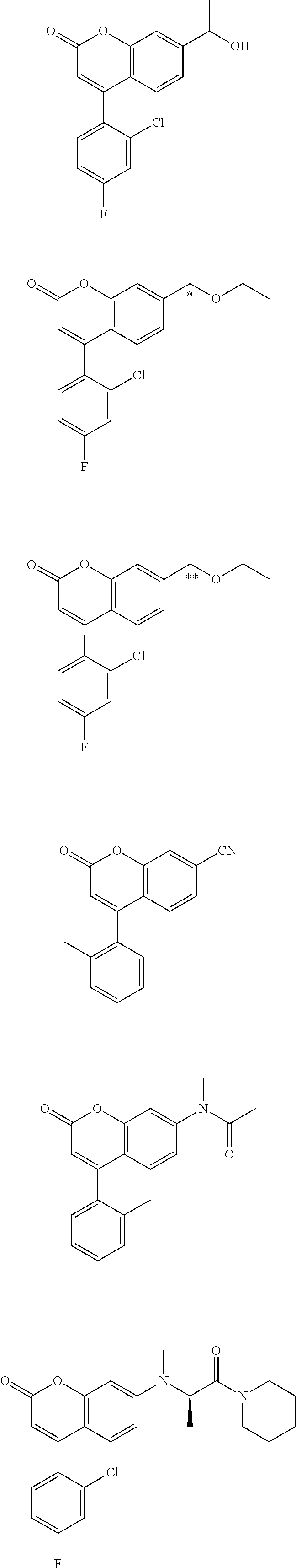 [SMILES: CC(O)c1ccc2c(-c3ccc(F)cc3Cl)cc(=O)oc2c1 ; CCOC(C)c1ccc2c(-c3ccc(F)cc3Cl)cc(=O)oc2c1 ; CCOC(C)c1ccc2c(-c3ccc(F)cc3Cl)cc(=O)oc2c1 ; Cc1ccccc1-c1cc(=O)oc2cc(C#N)ccc12 ; CC(=O)N(C)c1ccc2c(-c3ccccc3C)cc(=O)oc2c1 ; C[C@H](C(=O)N1CCCCC1)N(C)c1ccc2c(-c3ccc(F)cc3Cl)cc(=O)oc2c1] 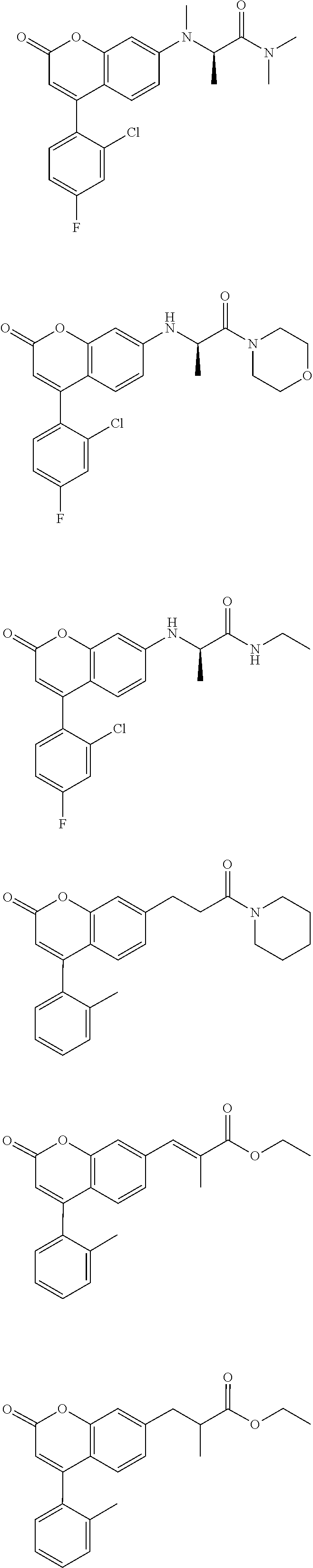 [SMILES: C[C@H](C(=O)N(C)C)N(C)c1ccc2c(-c3ccc(F)cc3Cl)cc(=O)oc2c1 ; C[C@@H](Nc1ccc2c(-c3ccc(F)cc3Cl)cc(=O)oc2c1)C(=O)N1CCOCC1 ; CCNC(=O)[C@@H](C)Nc1ccc2c(-c3ccc(F)cc3Cl)cc(=O)oc2c1 ; Cc1ccccc1-c1cc(=O)oc2cc(CCC(=O)N3CCCCC3)ccc12 ; CCOC(=O)/C(C)=C/c1ccc2c(-c3ccccc3C)cc(=O)oc2c1 ; CCOC(=O)C(C)Cc1ccc2c(-c3ccccc3C)cc(=O)oc2c1] 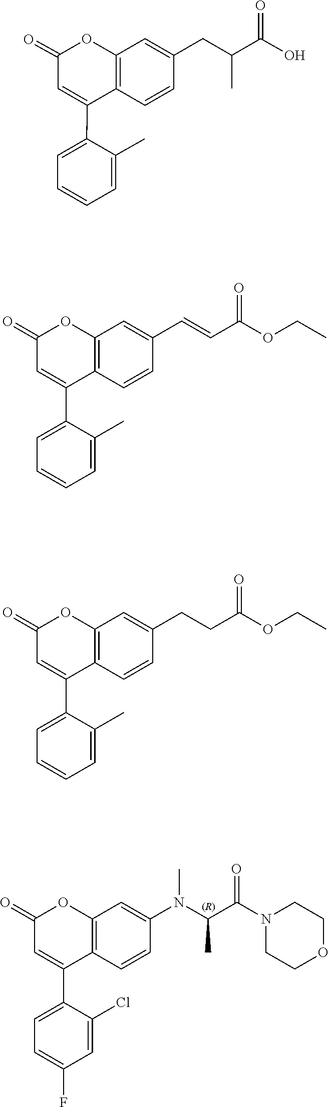 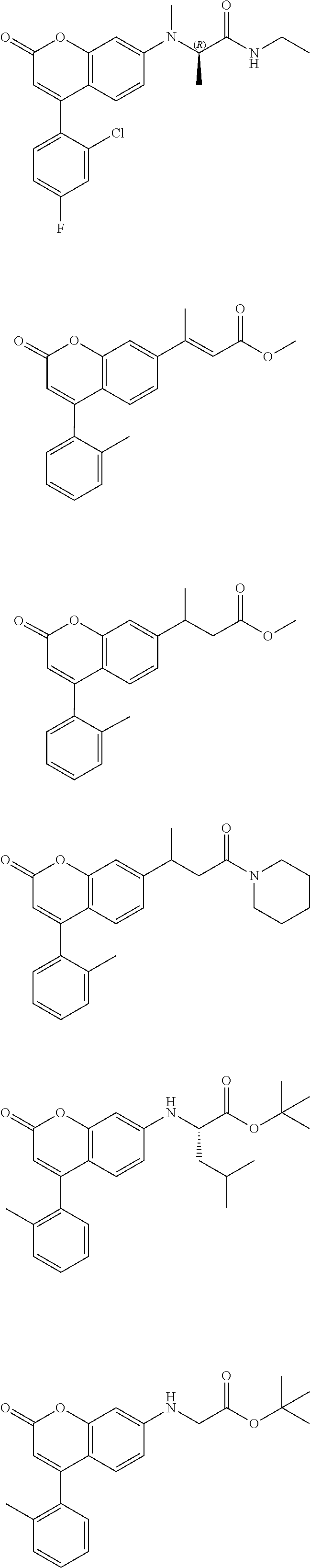 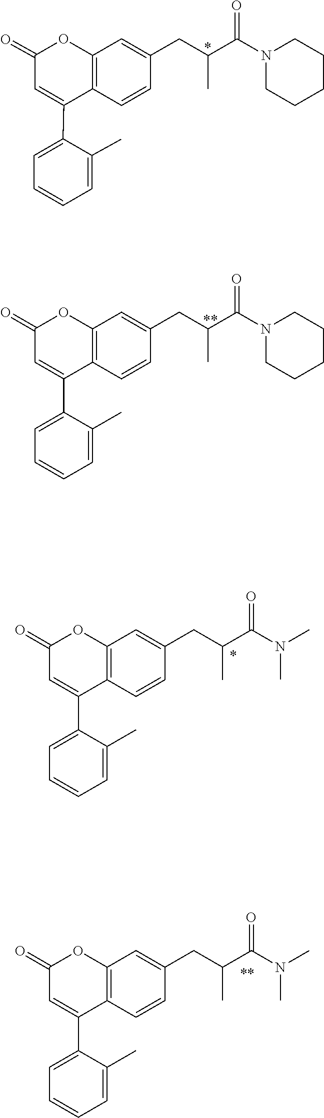 [SMILES: Cc1ccccc1-c1cc(=O)oc2cc(CC(C)C(=O)N3CCCCC3)ccc12 ; Cc1ccccc1-c1cc(=O)oc2cc(CC(C)C(=O)N3CCCCC3)ccc12 ; Cc1ccccc1-c1cc(=O)oc2cc(CC(C)C(=O)N(C)C)ccc12 ; Cc1ccccc1-c1cc(=O)oc2cc(CC(C)C(=O)N(C)C)ccc12] 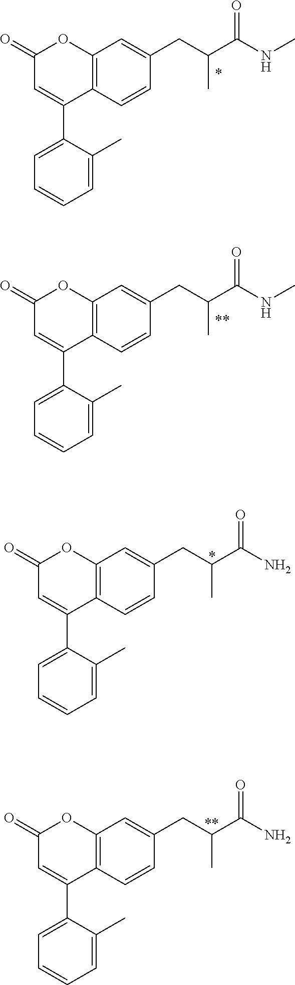 [SMILES: CNC(=O)C(C)Cc1ccc2c(-c3ccccc3C)cc(=O)oc2c1 ; CNC(=O)C(C)Cc1ccc2c(-c3ccccc3C)cc(=O)oc2c1 ; Cc1ccccc1-c1cc(=O)oc2cc(CC(C)C(N)=O)ccc12 ; Cc1ccccc1-c1cc(=O)oc2cc(CC(C)C(N)=O)ccc12] 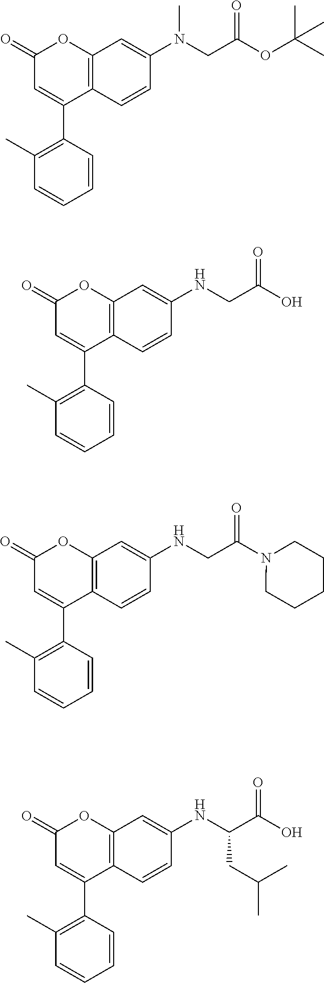 [SMILES: Cc1ccccc1-c1cc(=O)oc2cc(N(C)CC(=O)OC(C)(C)C)ccc12 ; Cc1ccccc1-c1cc(=O)oc2cc(NCC(=O)O)ccc12 ; Cc1ccccc1-c1cc(=O)oc2cc(NCC(=O)N3CCCCC3)ccc12 ; Cc1ccccc1-c1cc(=O)oc2cc(N[C@@H](CC(C)C)C(=O)O)ccc12] 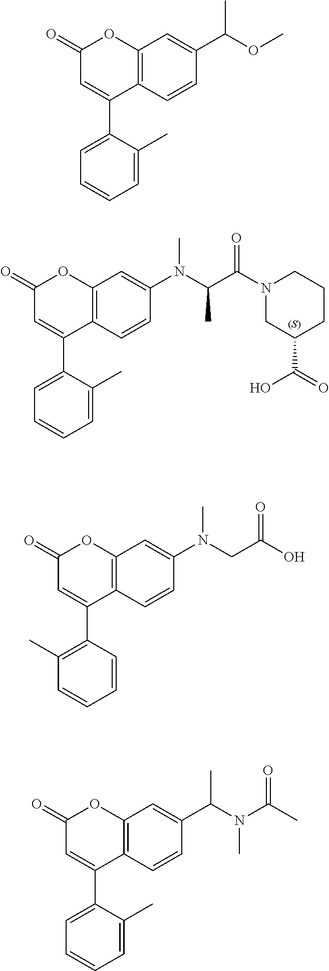 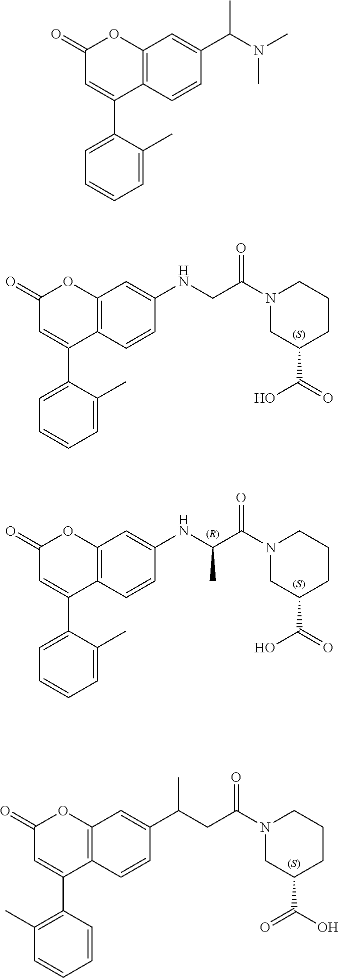 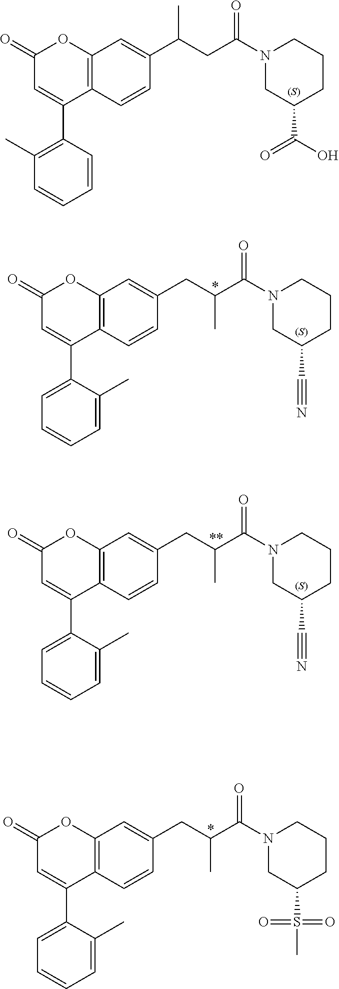 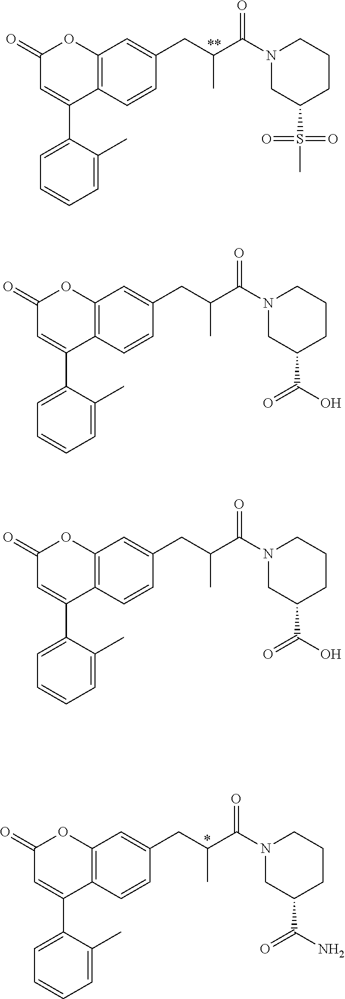 [SMILES: Cc1ccccc1-c1cc(=O)oc2cc(CC(C)C(=O)N3CCC[C@H](S(C)(=O)=O)C3)ccc12 ; Cc1ccccc1-c1cc(=O)oc2cc(CC(C)C(=O)N3CCC[C@H](C(=O)O)C3)ccc12 ; Cc1ccccc1-c1cc(=O)oc2cc(CC(C)C(=O)N3CCC[C@H](C(=O)O)C3)ccc12 ; Cc1ccccc1-c1cc(=O)oc2cc(CC(C)C(=O)N3CCC[C@H](C(N)=O)C3)ccc12] 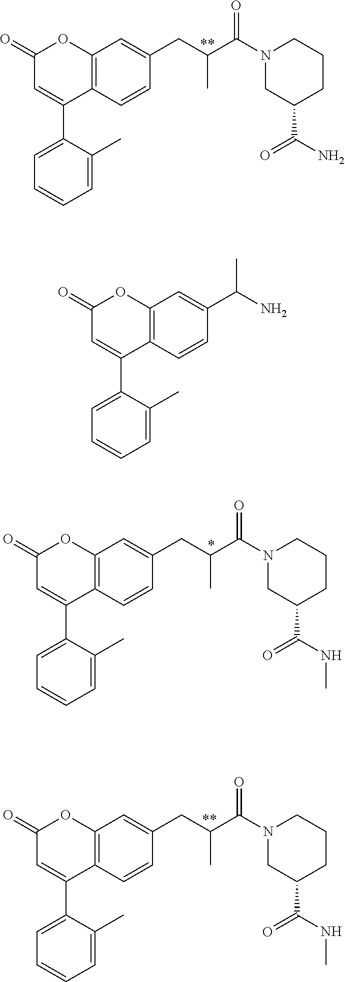 [SMILES: Cc1ccccc1-c1cc(=O)oc2cc(CC(C)C(=O)N3CCC[C@H](C(N)=O)C3)ccc12 ; Cc1ccccc1-c1cc(=O)oc2cc(C(C)N)ccc12 ; CNC(=O)[C@H]1CCCN(C(=O)C(C)Cc2ccc3c(-c4ccccc4C)cc(=O)oc3c2)C1 ; CNC(=O)[C@H]1CCCN(C(=O)C(C)Cc2ccc3c(-c4ccccc4C)cc(=O)oc3c2)C1] 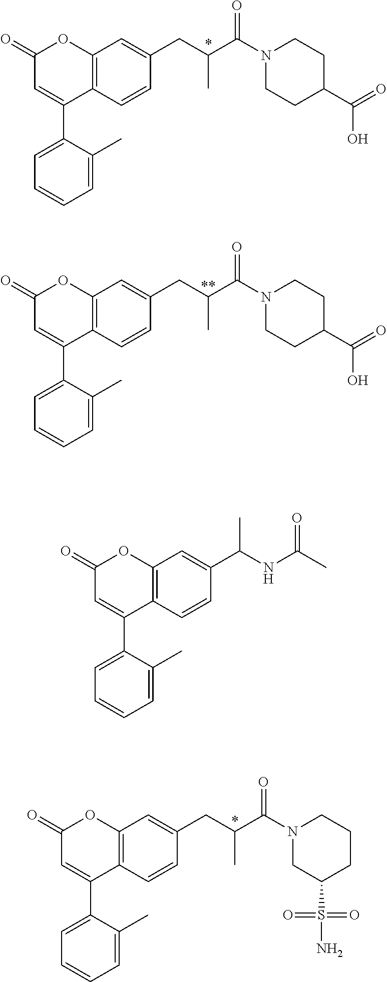 [SMILES: Cc1ccccc1-c1cc(=O)oc2cc(CC(C)C(=O)N3CCC(C(=O)O)CC3)ccc12 ; Cc1ccccc1-c1cc(=O)oc2cc(CC(C)C(=O)N3CCC(C(=O)O)CC3)ccc12 ; CC(=O)NC(C)c1ccc2c(-c3ccccc3C)cc(=O)oc2c1 ; Cc1ccccc1-c1cc(=O)oc2cc(CC(C)C(=O)N3CCC[C@H](S(N)(=O)=O)C3)ccc12] 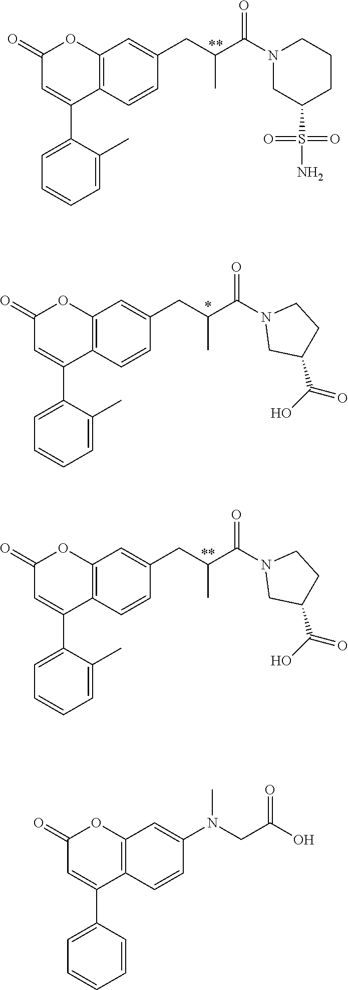 [SMILES: Cc1ccccc1-c1cc(=O)oc2cc(CC(C)C(=O)N3CCC[C@H](S(N)(=O)=O)C3)ccc12 ; Cc1ccccc1-c1cc(=O)oc2cc(CC(C)C(=O)N3CC[C@H](C(=O)O)C3)ccc12 ; Cc1ccccc1-c1cc(=O)oc2cc(CC(C)C(=O)N3CC[C@H](C(=O)O)C3)ccc12 ; CN(CC(=O)O)c1ccc2c(-c3ccccc3)cc(=O)oc2c1] 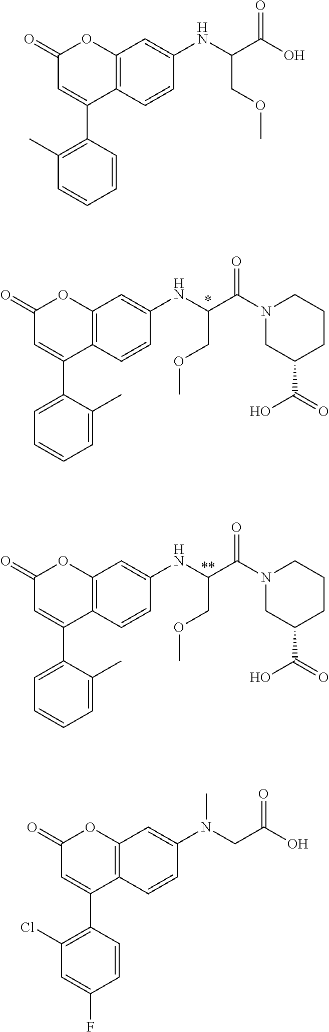 [SMILES: COCC(Nc1ccc2c(-c3ccccc3C)cc(=O)oc2c1)C(=O)O ; COCC(Nc1ccc2c(-c3ccccc3C)cc(=O)oc2c1)C(=O)N1CCC[C@H](C(=O)O)C1 ; COCC(Nc1ccc2c(-c3ccccc3C)cc(=O)oc2c1)C(=O)N1CCC[C@H](C(=O)O)C1 ; CN(CC(=O)O)c1ccc2c(-c3ccc(F)cc3Cl)cc(=O)oc2c1] 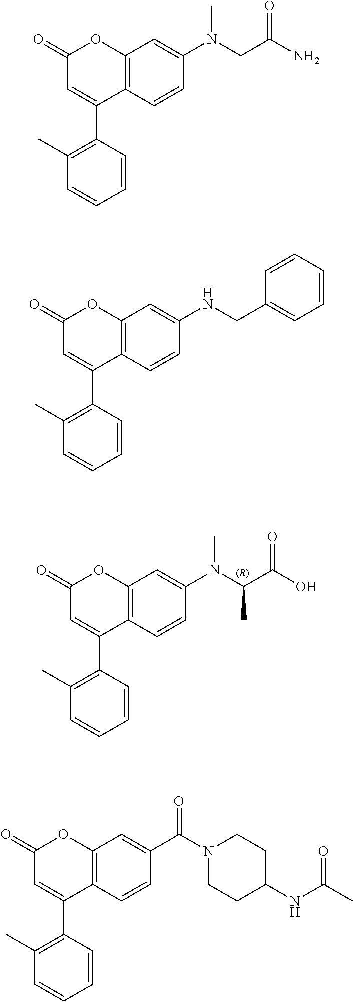 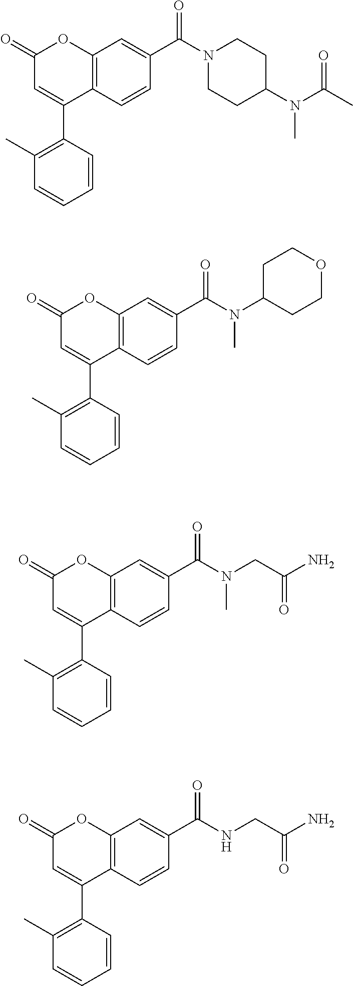 [SMILES: CC(=O)N(C)C1CCN(C(=O)c2ccc3c(-c4ccccc4C)cc(=O)oc3c2)CC1 ; Cc1ccccc1-c1cc(=O)oc2cc(C(=O)N(C)C3CCOCC3)ccc12 ; Cc1ccccc1-c1cc(=O)oc2cc(C(=O)N(C)CC(N)=O)ccc12 ; Cc1ccccc1-c1cc(=O)oc2cc(C(=O)NCC(N)=O)ccc12] 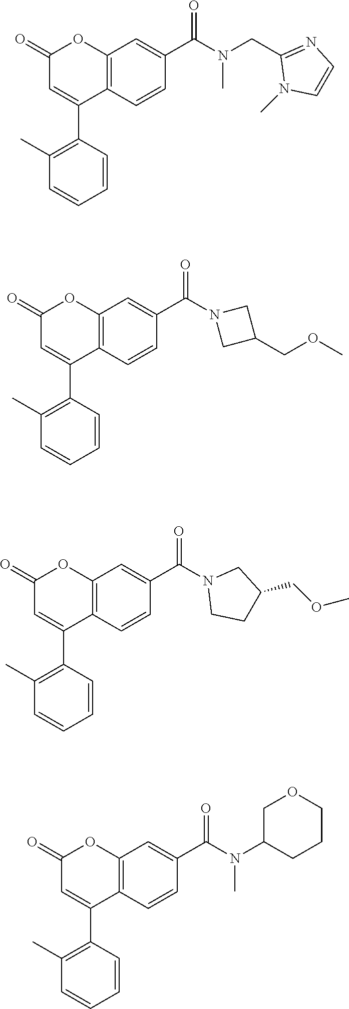 [SMILES: Cc1ccccc1-c1cc(=O)oc2cc(C(=O)N(C)Cc3nccn3C)ccc12 ; COCC1CN(C(=O)c2ccc3c(-c4ccccc4C)cc(=O)oc3c2)C1 ; COC[C@@H]1CCN(C(=O)c2ccc3c(-c4ccccc4C)cc(=O)oc3c2)C1 ; Cc1ccccc1-c1cc(=O)oc2cc(C(=O)N(C)C3CCCOC3)ccc12] 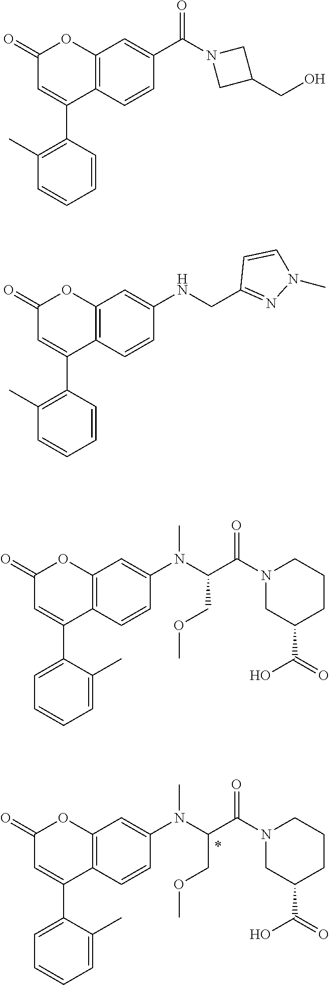 [SMILES: Cc1ccccc1-c1cc(=O)oc2cc(C(=O)N3CC(CO)C3)ccc12 ; Cc1ccccc1-c1cc(=O)oc2cc(NCc3ccn(C)n3)ccc12 ; COC[C@@H](C(=O)N1CCC[C@H](C(=O)O)C1)N(C)c1ccc2c(-c3ccccc3C)cc(=O)oc2c1 ; COCC(C(=O)N1CCC[C@H](C(=O)O)C1)N(C)c1ccc2c(-c3ccccc3C)cc(=O)oc2c1] 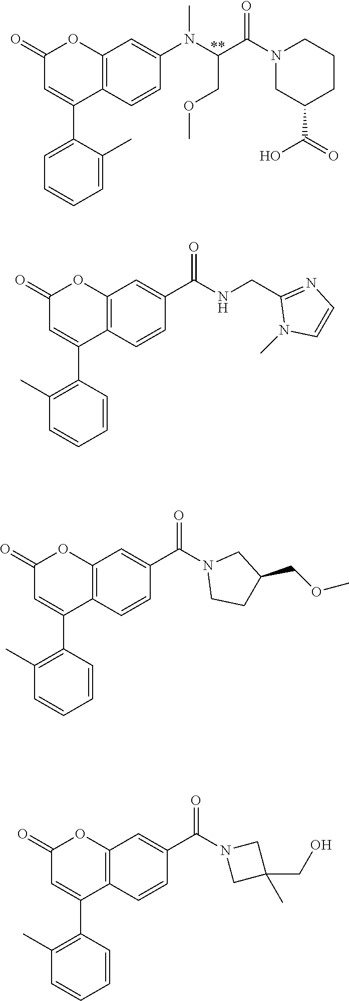 [SMILES: COCC(C(=O)N1CCC[C@H](C(=O)O)C1)N(C)c1ccc2c(-c3ccccc3C)cc(=O)oc2c1 ; Cc1ccccc1-c1cc(=O)oc2cc(C(=O)NCc3nccn3C)ccc12 ; COC[C@H]1CCN(C(=O)c2ccc3c(-c4ccccc4C)cc(=O)oc3c2)C1 ; Cc1ccccc1-c1cc(=O)oc2cc(C(=O)N3CC(C)(CO)C3)ccc12] 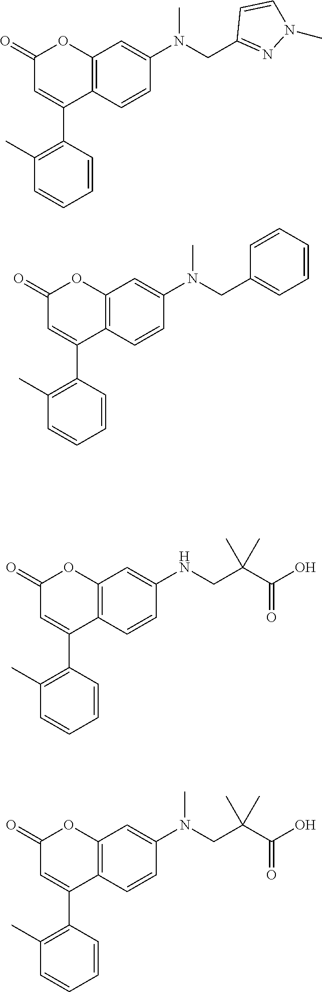 [SMILES: Cc1ccccc1-c1cc(=O)oc2cc(N(C)Cc3ccn(C)n3)ccc12 ; Cc1ccccc1-c1cc(=O)oc2cc(N(C)Cc3ccccc3)ccc12 ; Cc1ccccc1-c1cc(=O)oc2cc(NCC(C)(C)C(=O)O)ccc12 ; Cc1ccccc1-c1cc(=O)oc2cc(N(C)CC(C)(C)C(=O)O)ccc12] 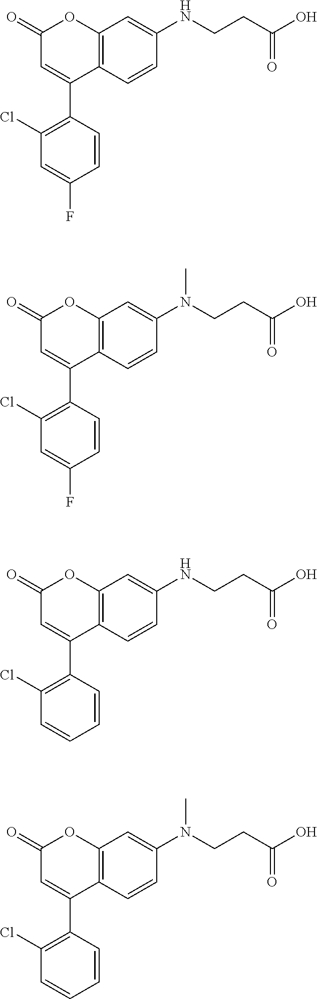 [SMILES: O=C(O)CCNc1ccc2c(-c3ccc(F)cc3Cl)cc(=O)oc2c1 ; CN(CCC(=O)O)c1ccc2c(-c3ccc(F)cc3Cl)cc(=O)oc2c1 ; O=C(O)CCNc1ccc2c(-c3ccccc3Cl)cc(=O)oc2c1 ; CN(CCC(=O)O)c1ccc2c(-c3ccccc3Cl)cc(=O)oc2c1] 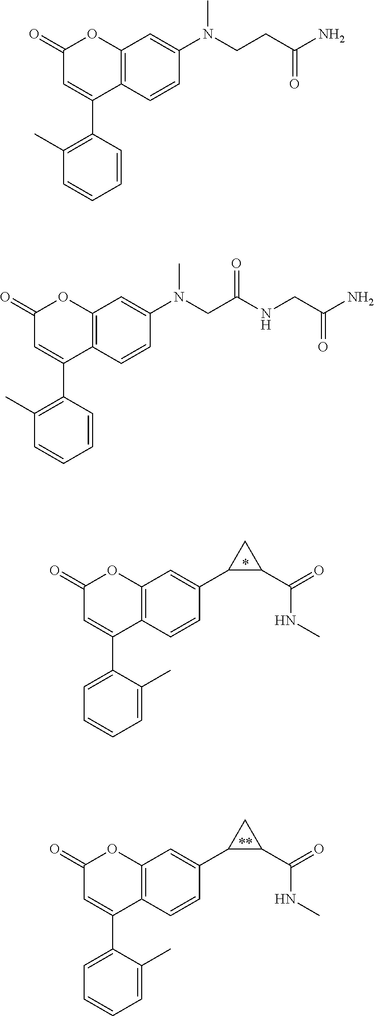 [SMILES: Cc1ccccc1-c1cc(=O)oc2cc(N(C)CCC(N)=O)ccc12 ; Cc1ccccc1-c1cc(=O)oc2cc(N(C)CC(=O)NCC(N)=O)ccc12 ; CNC(=O)C1CC1c1ccc2c(-c3ccccc3C)cc(=O)oc2c1 ; CNC(=O)C1CC1c1ccc2c(-c3ccccc3C)cc(=O)oc2c1] 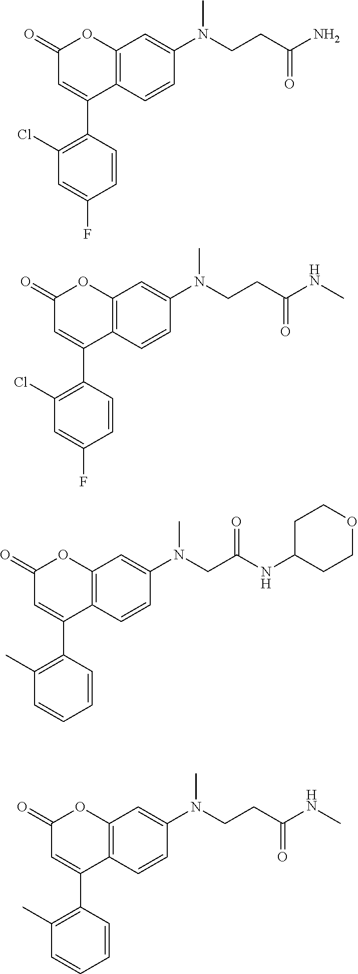 [SMILES: CN(CCC(N)=O)c1ccc2c(-c3ccc(F)cc3Cl)cc(=O)oc2c1 ; CNC(=O)CCN(C)c1ccc2c(-c3ccc(F)cc3Cl)cc(=O)oc2c1 ; Cc1ccccc1-c1cc(=O)oc2cc(N(C)CC(=O)NC3CCOCC3)ccc12 ; CNC(=O)CCN(C)c1ccc2c(-c3ccccc3C)cc(=O)oc2c1] 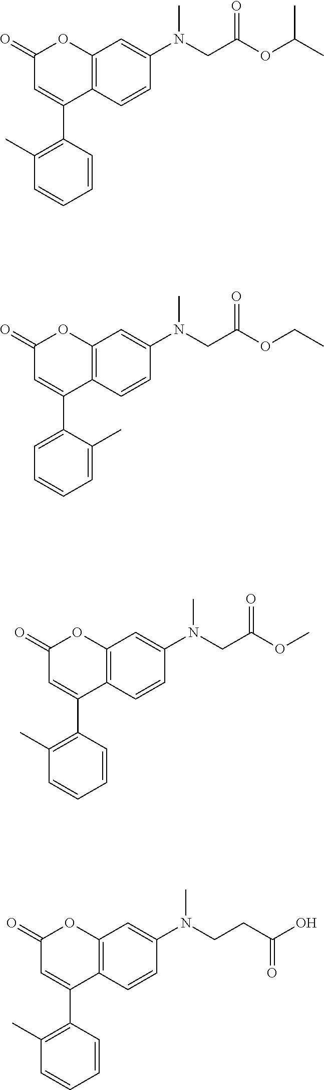 [SMILES: Cc1ccccc1-c1cc(=O)oc2cc(N(C)CC(=O)OC(C)C)ccc12 ; CCOC(=O)CN(C)c1ccc2c(-c3ccccc3C)cc(=O)oc2c1 ; COC(=O)CN(C)c1ccc2c(-c3ccccc3C)cc(=O)oc2c1 ; Cc1ccccc1-c1cc(=O)oc2cc(N(C)CCC(=O)O)ccc12] 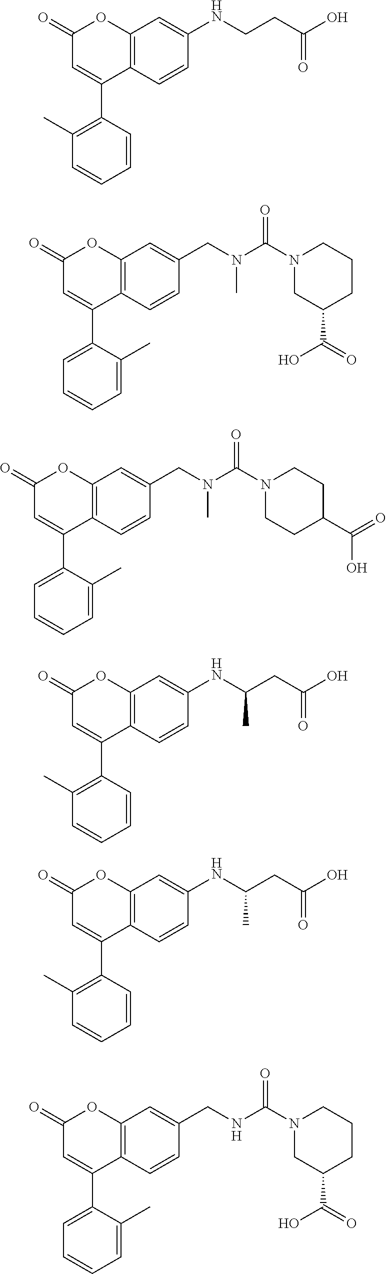 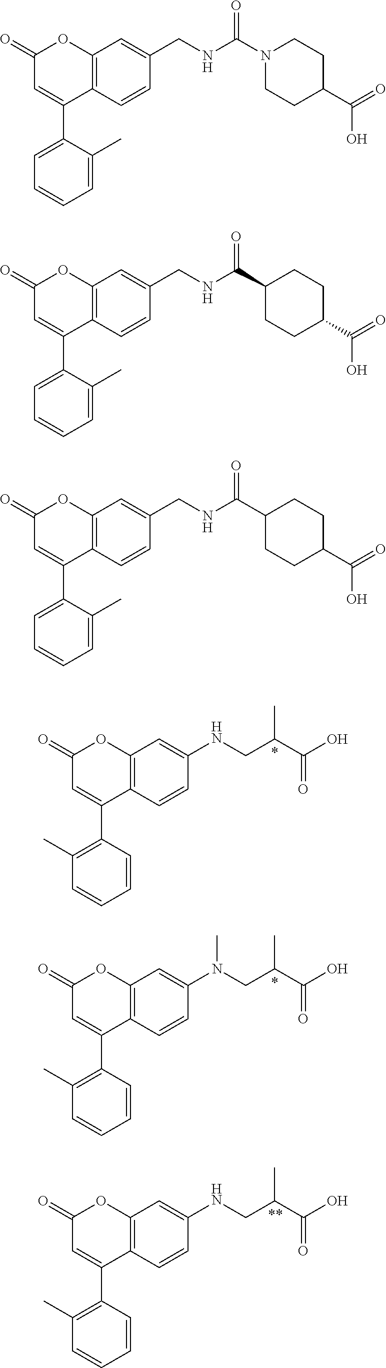 [SMILES: Cc1ccccc1-c1cc(=O)oc2cc(CNC(=O)N3CCC(C(=O)O)CC3)ccc12 ; Cc1ccccc1-c1cc(=O)oc2cc(CNC(=O)[C@H]3CC[C@H](C(=O)O)CC3)ccc12 ; Cc1ccccc1-c1cc(=O)oc2cc(CNC(=O)C3CCC(C(=O)O)CC3)ccc12 ; Cc1ccccc1-c1cc(=O)oc2cc(NCC(C)C(=O)O)ccc12 ; Cc1ccccc1-c1cc(=O)oc2cc(N(C)CC(C)C(=O)O)ccc12 ; Cc1ccccc1-c1cc(=O)oc2cc(NCC(C)C(=O)O)ccc12] 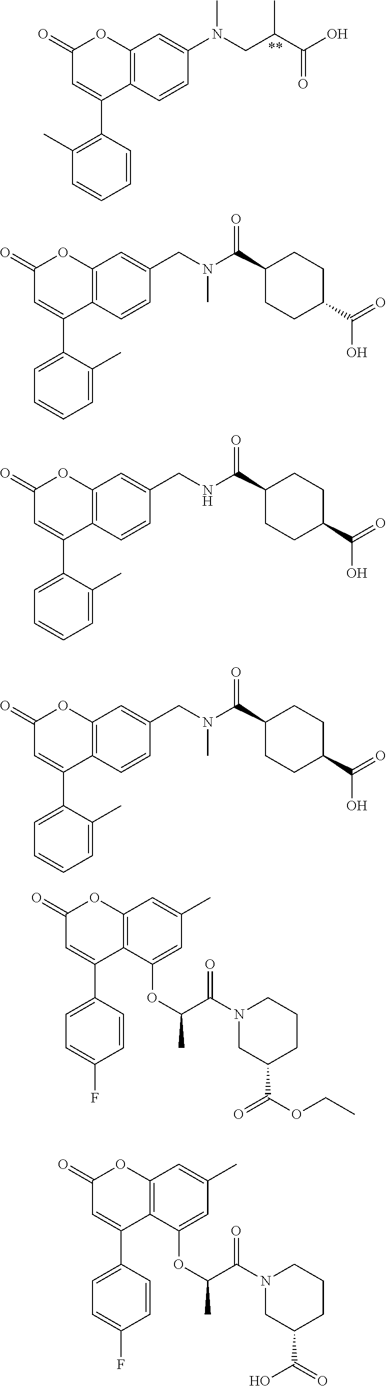 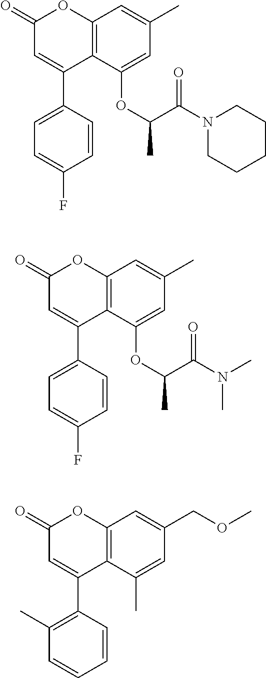 [SMILES: Cc1cc(O[C@H](C)C(=O)N2CCCCC2)c2c(-c3ccc(F)cc3)cc(=O)oc2c1 ; Cc1cc(O[C@H](C)C(=O)N(C)C)c2c(-c3ccc(F)cc3)cc(=O)oc2c1 ; COCc1cc(C)c2c(-c3ccccc3C)cc(=O)oc2c1] 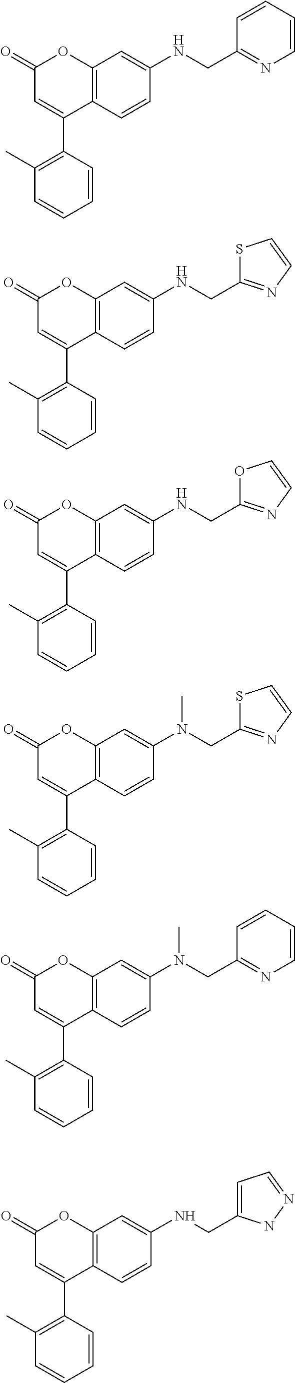 [SMILES: Cc1ccccc1-c1cc(=O)oc2cc(NCc3ccccn3)ccc12 ; Cc1ccccc1-c1cc(=O)oc2cc(NCc3nccs3)ccc12 ; Cc1ccccc1-c1cc(=O)oc2cc(NCc3ncco3)ccc12 ; Cc1ccccc1-c1cc(=O)oc2cc(N(C)Cc3nccs3)ccc12 ; Cc1ccccc1-c1cc(=O)oc2cc(N(C)Cc3ccccn3)ccc12 ; Cc1ccccc1-c1cc(=O)oc2cc(NCc3ccn[nH]3)ccc12] 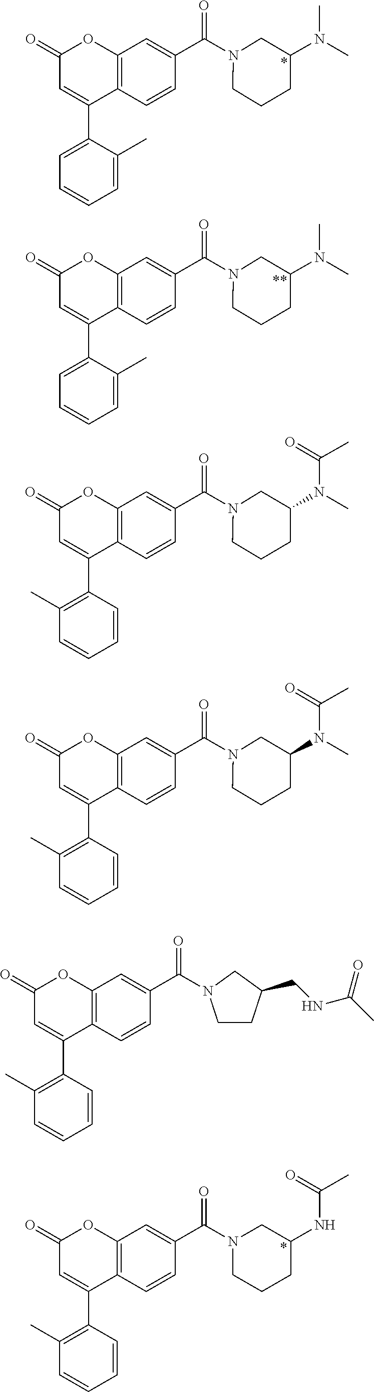 [SMILES: Cc1ccccc1-c1cc(=O)oc2cc(C(=O)N3CCCC(N(C)C)C3)ccc12 ; Cc1ccccc1-c1cc(=O)oc2cc(C(=O)N3CCCC(N(C)C)C3)ccc12 ; CC(=O)N(C)[C@@H]1CCCN(C(=O)c2ccc3c(-c4ccccc4C)cc(=O)oc3c2)C1 ; CC(=O)N(C)[C@H]1CCCN(C(=O)c2ccc3c(-c4ccccc4C)cc(=O)oc3c2)C1 ; CC(=O)NC[C@H]1CCN(C(=O)c2ccc3c(-c4ccccc4C)cc(=O)oc3c2)C1 ; CC(=O)NC1CCCN(C(=O)c2ccc3c(-c4ccccc4C)cc(=O)oc3c2)C1] 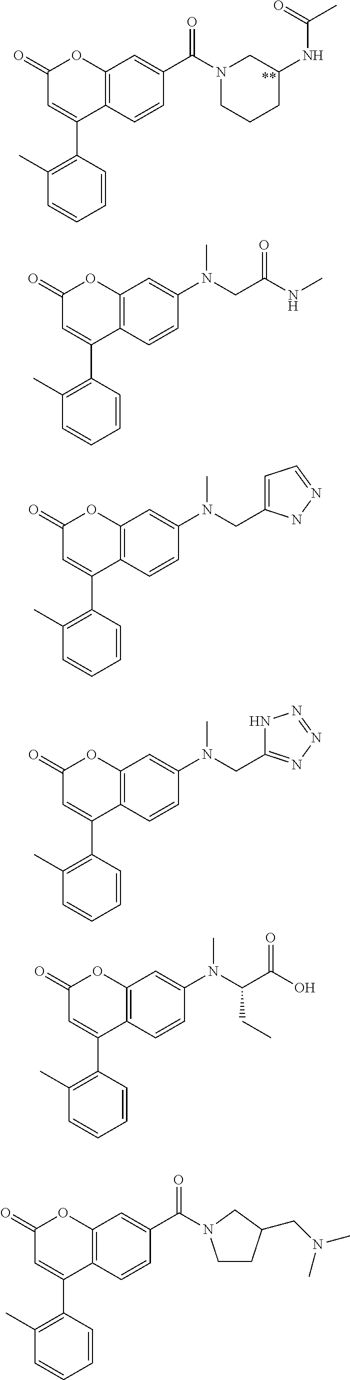 [SMILES: CC(=O)NC1CCCN(C(=O)c2ccc3c(-c4ccccc4C)cc(=O)oc3c2)C1 ; CNC(=O)CN(C)c1ccc2c(-c3ccccc3C)cc(=O)oc2c1 ; Cc1ccccc1-c1cc(=O)oc2cc(N(C)Cc3ccn[nH]3)ccc12 ; Cc1ccccc1-c1cc(=O)oc2cc(N(C)Cc3nnn[nH]3)ccc12 ; CC[C@@H](C(=O)O)N(C)c1ccc2c(-c3ccccc3C)cc(=O)oc2c1 ; Cc1ccccc1-c1cc(=O)oc2cc(C(=O)N3CCC(CN(C)C)C3)ccc12] 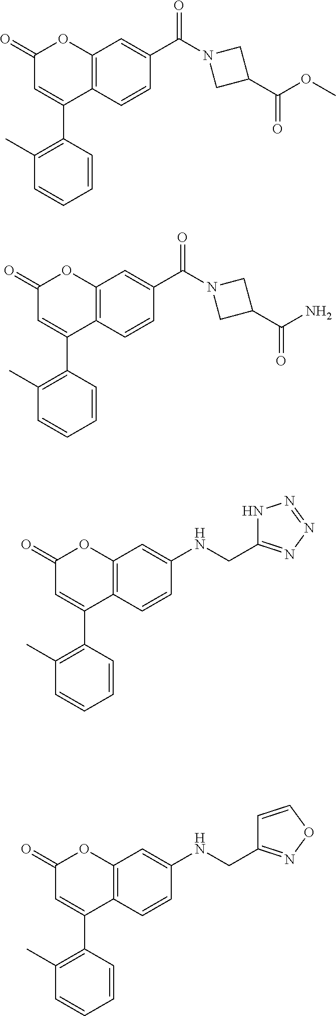 [SMILES: COC(=O)C1CN(C(=O)c2ccc3c(-c4ccccc4C)cc(=O)oc3c2)C1 ; Cc1ccccc1-c1cc(=O)oc2cc(C(=O)N3CC(C(N)=O)C3)ccc12 ; Cc1ccccc1-c1cc(=O)oc2cc(NCc3nnn[nH]3)ccc12 ; Cc1ccccc1-c1cc(=O)oc2cc(NCc3ccon3)ccc12] 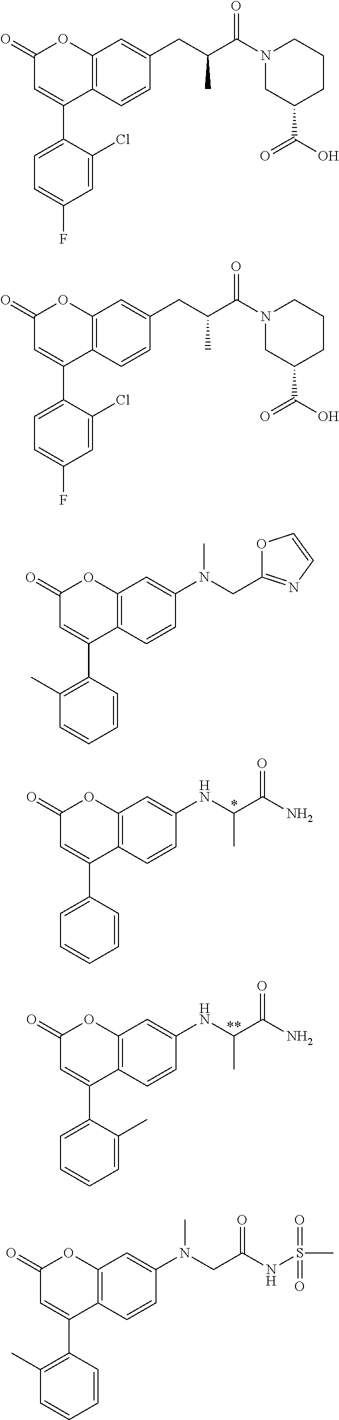 [SMILES: C[C@@H](Cc1ccc2c(-c3ccc(F)cc3Cl)cc(=O)oc2c1)C(=O)N1CCC[C@H](C(=O)O)C1 ; C[C@H](Cc1ccc2c(-c3ccc(F)cc3Cl)cc(=O)oc2c1)C(=O)N1CCC[C@H](C(=O)O)C1 ; Cc1ccccc1-c1cc(=O)oc2cc(N(C)Cc3ncco3)ccc12 ; CC(Nc1ccc2c(-c3ccccc3)cc(=O)oc2c1)C(N)=O ; Cc1ccccc1-c1cc(=O)oc2cc(NC(C)C(N)=O)ccc12 ; Cc1ccccc1-c1cc(=O)oc2cc(N(C)CC(=O)NS(C)(=O)=O)ccc12] 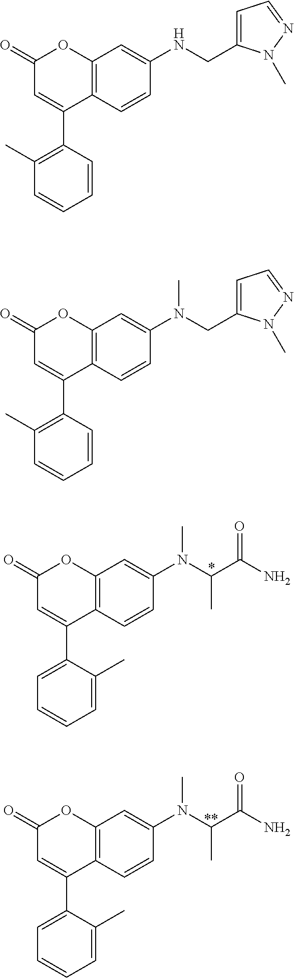 [SMILES: Cc1ccccc1-c1cc(=O)oc2cc(NCc3ccnn3C)ccc12 ; Cc1ccccc1-c1cc(=O)oc2cc(N(C)Cc3ccnn3C)ccc12 ; Cc1ccccc1-c1cc(=O)oc2cc(N(C)C(C)C(N)=O)ccc12 ; Cc1ccccc1-c1cc(=O)oc2cc(N(C)C(C)C(N)=O)ccc12] 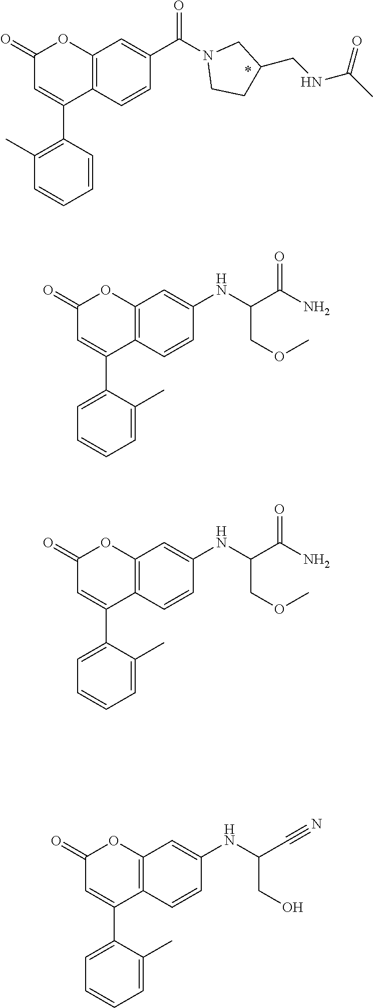 [SMILES: CC(=O)NCC1CCN(C(=O)c2ccc3c(-c4ccccc4C)cc(=O)oc3c2)C1 ; COCC(Nc1ccc2c(-c3ccccc3C)cc(=O)oc2c1)C(N)=O ; COCC(Nc1ccc2c(-c3ccccc3C)cc(=O)oc2c1)C(N)=O ; Cc1ccccc1-c1cc(=O)oc2cc(NC(C#N)CO)ccc12] 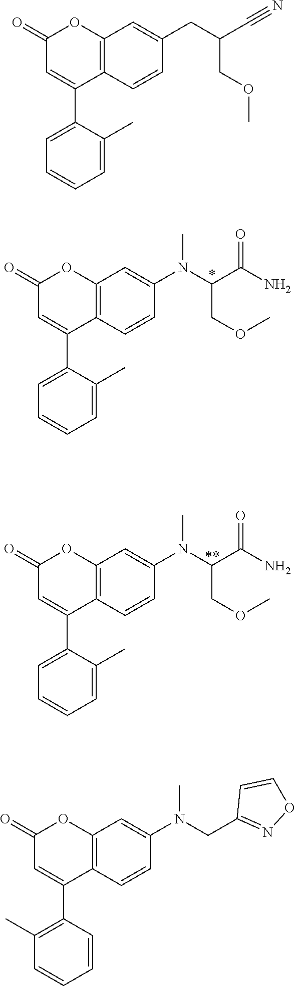 [SMILES: COCC(C#N)Cc1ccc2c(-c3ccccc3C)cc(=O)oc2c1 ; COCC(C(N)=O)N(C)c1ccc2c(-c3ccccc3C)cc(=O)oc2c1 ; COCC(C(N)=O)N(C)c1ccc2c(-c3ccccc3C)cc(=O)oc2c1 ; Cc1ccccc1-c1cc(=O)oc2cc(N(C)Cc3ccon3)ccc12] 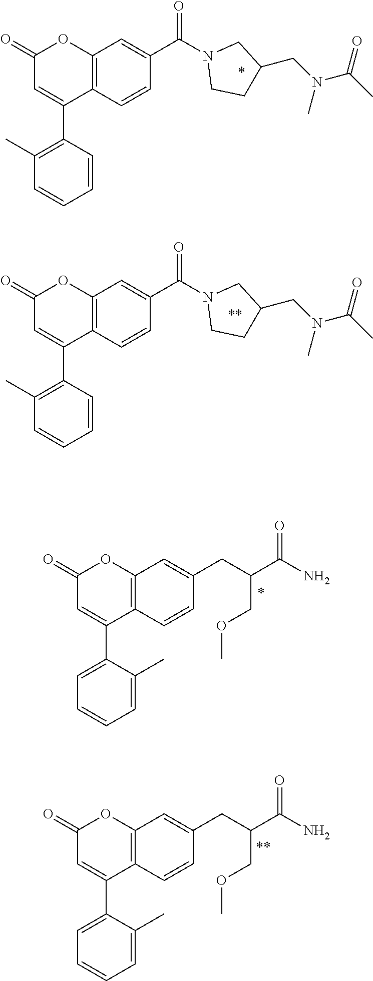 [SMILES: CC(=O)N(C)CC1CCN(C(=O)c2ccc3c(-c4ccccc4C)cc(=O)oc3c2)C1 ; CC(=O)N(C)CC1CCN(C(=O)c2ccc3c(-c4ccccc4C)cc(=O)oc3c2)C1 ; COCC(Cc1ccc2c(-c3ccccc3C)cc(=O)oc2c1)C(N)=O ; COCC(Cc1ccc2c(-c3ccccc3C)cc(=O)oc2c1)C(N)=O] 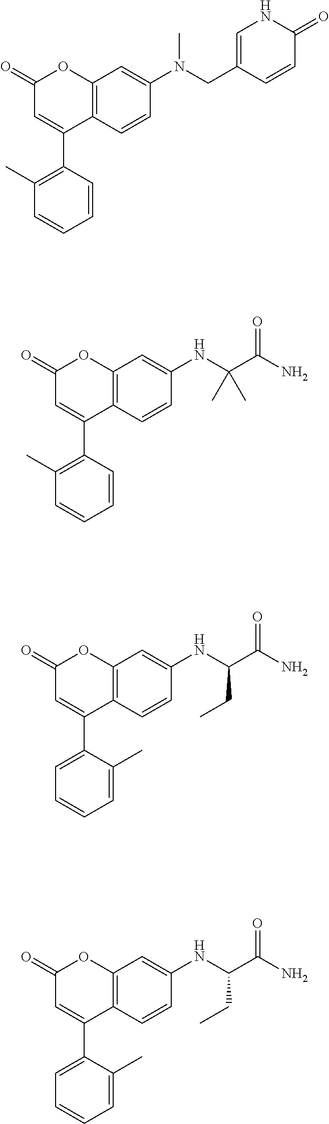 [SMILES: Cc1ccccc1-c1cc(=O)oc2cc(N(C)Cc3ccc(=O)[nH]c3)ccc12 ; Cc1ccccc1-c1cc(=O)oc2cc(NC(C)(C)C(N)=O)ccc12 ; CC[C@@H](Nc1ccc2c(-c3ccccc3C)cc(=O)oc2c1)C(N)=O ; CC[C@H](Nc1ccc2c(-c3ccccc3C)cc(=O)oc2c1)C(N)=O] 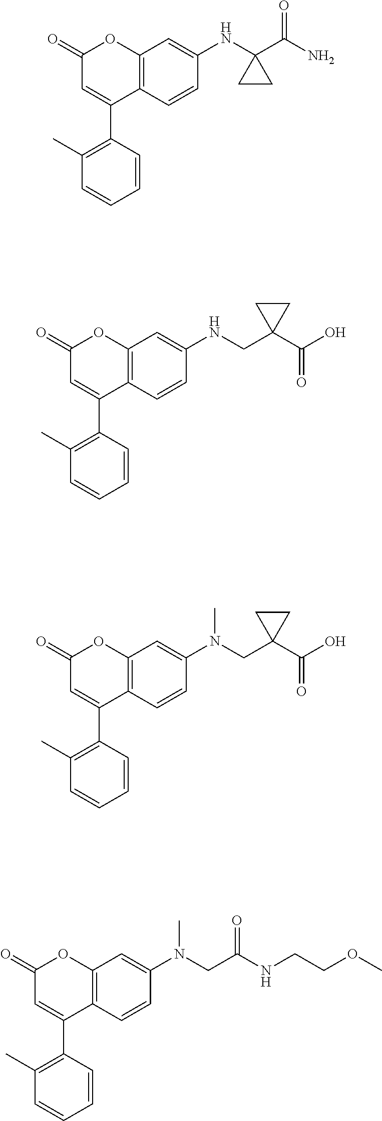 [SMILES: Cc1ccccc1-c1cc(=O)oc2cc(NC3(C(N)=O)CC3)ccc12 ; Cc1ccccc1-c1cc(=O)oc2cc(NCC3(C(=O)O)CC3)ccc12 ; Cc1ccccc1-c1cc(=O)oc2cc(N(C)CC3(C(=O)O)CC3)ccc12 ; COCCNC(=O)CN(C)c1ccc2c(-c3ccccc3C)cc(=O)oc2c1] 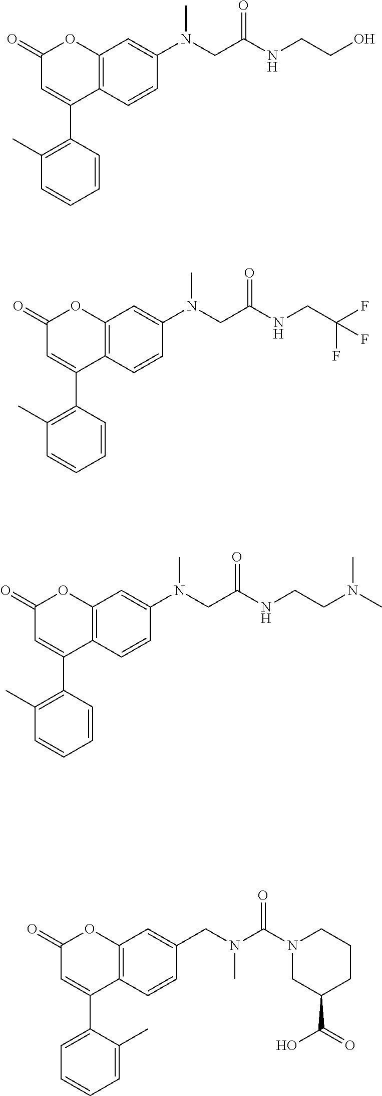 [SMILES: Cc1ccccc1-c1cc(=O)oc2cc(N(C)CC(=O)NCCO)ccc12 ; Cc1ccccc1-c1cc(=O)oc2cc(N(C)CC(=O)NCC(F)(F)F)ccc12 ; Cc1ccccc1-c1cc(=O)oc2cc(N(C)CC(=O)NCCN(C)C)ccc12 ; Cc1ccccc1-c1cc(=O)oc2cc(CN(C)C(=O)N3CCC[C@@H](C(=O)O)C3)ccc12] 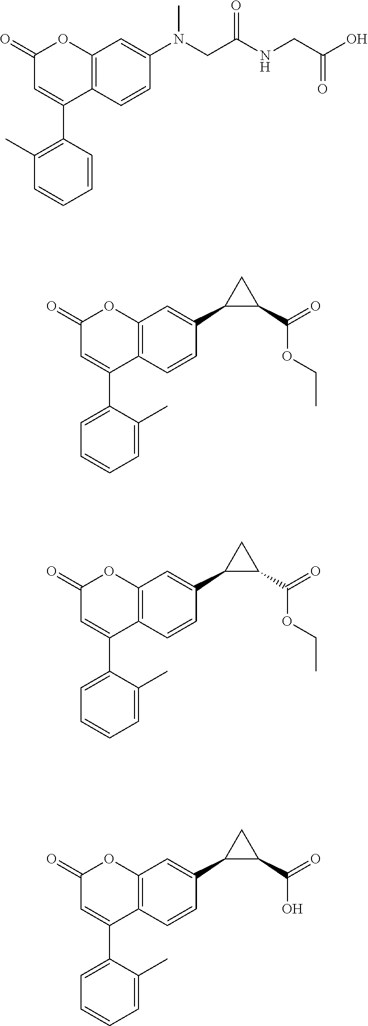 [SMILES: Cc1ccccc1-c1cc(=O)oc2cc(N(C)CC(=O)NCC(=O)O)ccc12 ; CCOC(=O)[C@@H]1C[C@@H]1c1ccc2c(-c3ccccc3C)cc(=O)oc2c1 ; CCOC(=O)[C@H]1C[C@@H]1c1ccc2c(-c3ccccc3C)cc(=O)oc2c1 ; Cc1ccccc1-c1cc(=O)oc2cc([C@H]3C[C@H]3C(=O)O)ccc12] 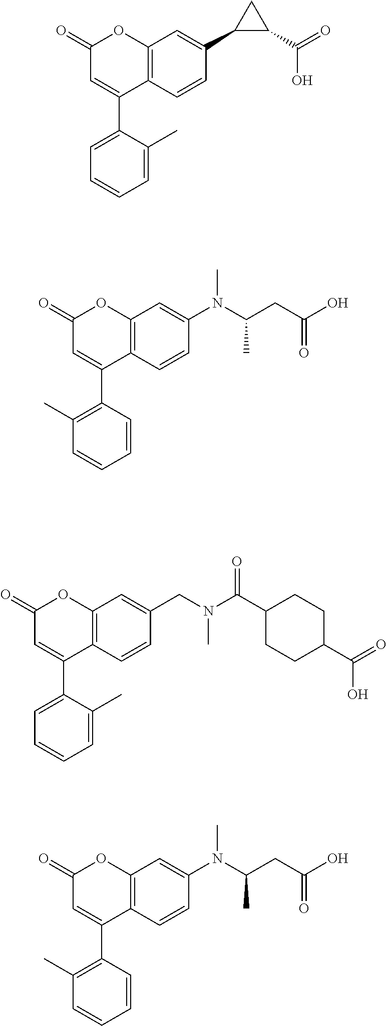 [SMILES: Cc1ccccc1-c1cc(=O)oc2cc([C@H]3C[C@@H]3C(=O)O)ccc12 ; Cc1ccccc1-c1cc(=O)oc2cc(N(C)[C@@H](C)CC(=O)O)ccc12 ; Cc1ccccc1-c1cc(=O)oc2cc(CN(C)C(=O)C3CCC(C(=O)O)CC3)ccc12 ; Cc1ccccc1-c1cc(=O)oc2cc(N(C)[C@H](C)CC(=O)O)ccc12] 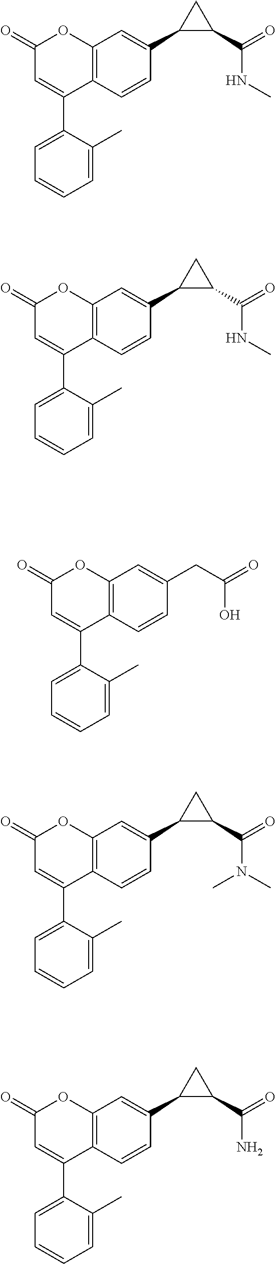 [SMILES: CNC(=O)[C@@H]1C[C@@H]1c1ccc2c(-c3ccccc3C)cc(=O)oc2c1 ; CNC(=O)[C@H]1C[C@@H]1c1ccc2c(-c3ccccc3C)cc(=O)oc2c1 ; Cc1ccccc1-c1cc(=O)oc2cc(CC(=O)O)ccc12 ; Cc1ccccc1-c1cc(=O)oc2cc([C@H]3C[C@H]3C(=O)N(C)C)ccc12 ; Cc1ccccc1-c1cc(=O)oc2cc([C@H]3C[C@H]3C(N)=O)ccc12] 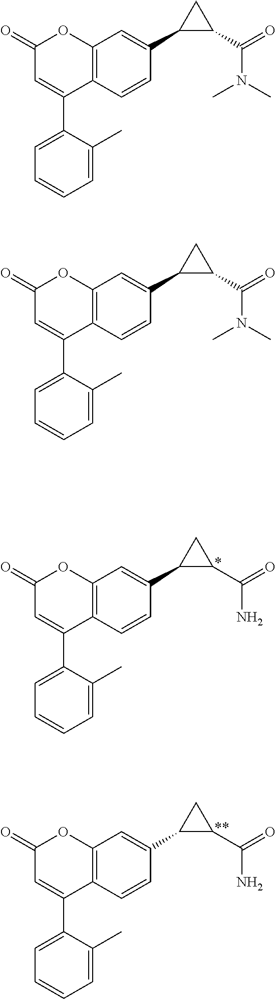 [SMILES: Cc1ccccc1-c1cc(=O)oc2cc([C@H]3C[C@@H]3C(=O)N(C)C)ccc12 ; Cc1ccccc1-c1cc(=O)oc2cc([C@H]3C[C@@H]3C(=O)N(C)C)ccc12 ; Cc1ccccc1-c1cc(=O)oc2cc([C@H]3CC3C(N)=O)ccc12 ; Cc1ccccc1-c1cc(=O)oc2cc([C@@H]3CC3C(N)=O)ccc12] 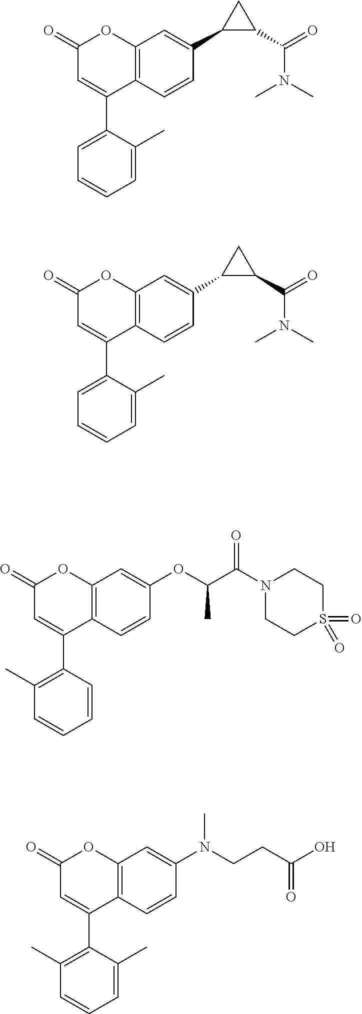 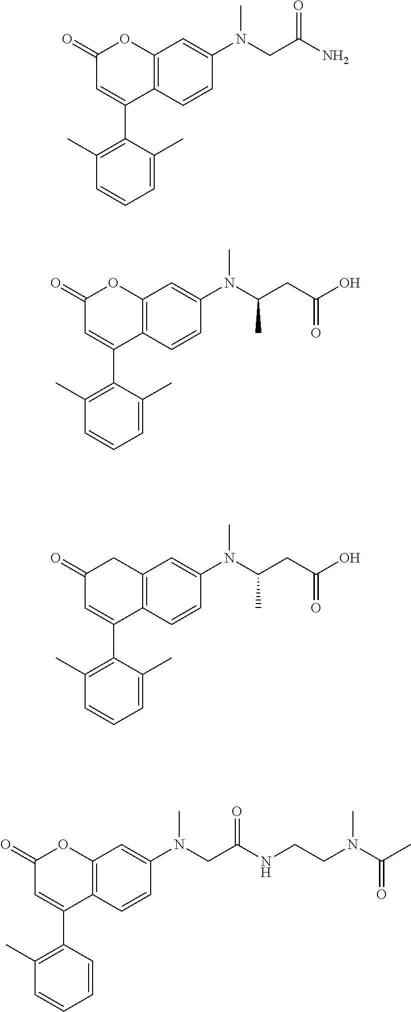 [SMILES: Cc1cccc(C)c1-c1cc(=O)oc2cc(N(C)CC(N)=O)ccc12 ; Cc1cccc(C)c1-c1cc(=O)oc2cc(N(C)[C@H](C)CC(=O)O)ccc12 ; Cc1cccc(C)c1C1=CC(=O)Cc2cc(N(C)[C@@H](C)CC(=O)O)ccc21 ; CC(=O)N(C)CCNC(=O)CN(C)c1ccc2c(-c3ccccc3C)cc(=O)oc2c1] 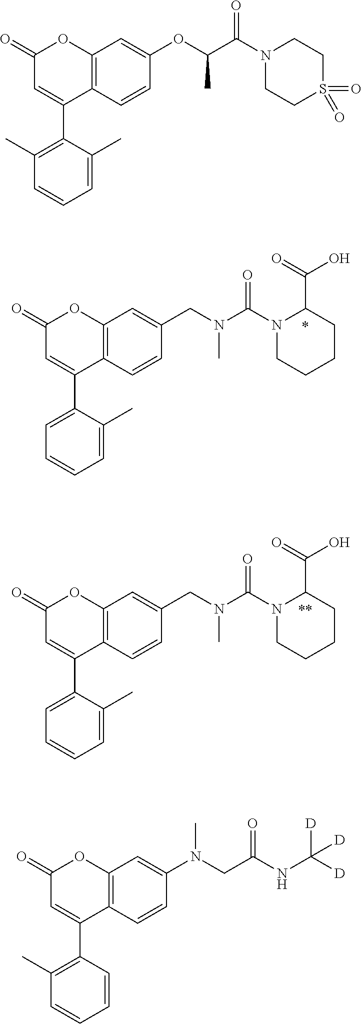 [SMILES: Cc1cccc(C)c1-c1cc(=O)oc2cc(O[C@H](C)C(=O)N3CCS(=O)(=O)CC3)ccc12 ; Cc1ccccc1-c1cc(=O)oc2cc(CN(C)C(=O)N3CCCCC3C(=O)O)ccc12 ; Cc1ccccc1-c1cc(=O)oc2cc(CN(C)C(=O)N3CCCCC3C(=O)O)ccc12 ; [2H]C([2H])([2H])NC(=O)CN(C)c1ccc2c(-c3ccccc3C)cc(=O)oc2c1]
or a pharmaceutically acceptable salt thereof.

20. The method according to claim 16, wherein the compound is selected from the group consisting of: 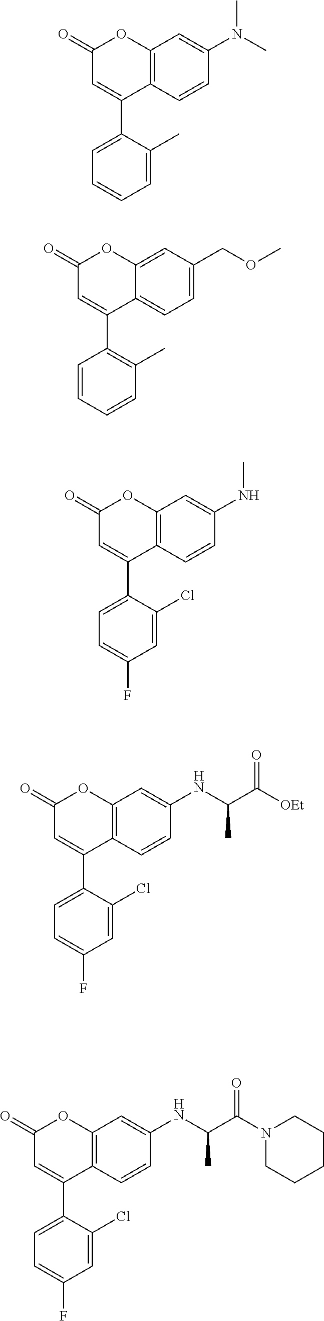 [SMILES: Cc1ccccc1-c1cc(=O)oc2cc(N(C)C)ccc12 ; COCc1ccc2c(-c3ccccc3C)cc(=O)oc2c1 ; CNc1ccc2c(-c3ccc(F)cc3Cl)cc(=O)oc2c1 ; CCOC(=O)[C@@H](C)Nc1ccc2c(-c3ccc(F)cc3Cl)cc(=O)oc2c1 ; C[C@@H](Nc1ccc2c(-c3ccc(F)cc3Cl)cc(=O)oc2c1)C(=O)N1CCCCC1] 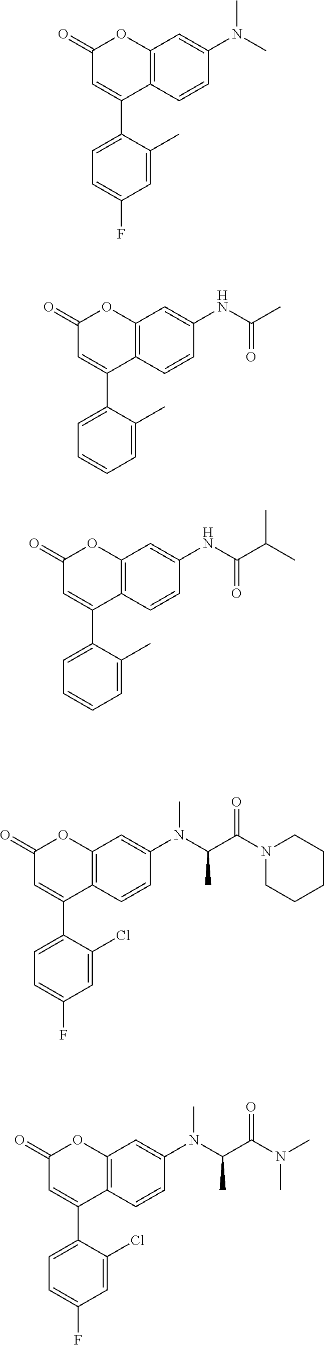 [SMILES: Cc1cc(F)ccc1-c1cc(=O)oc2cc(N(C)C)ccc12 ; CC(=O)Nc1ccc2c(-c3ccccc3C)cc(=O)oc2c1 ; Cc1ccccc1-c1cc(=O)oc2cc(NC(=O)C(C)C)ccc12 ; C[C@H](C(=O)N1CCCCC1)N(C)c1ccc2c(-c3ccc(F)cc3Cl)cc(=O)oc2c1 ; C[C@H](C(=O)N(C)C)N(C)c1ccc2c(-c3ccc(F)cc3Cl)cc(=O)oc2c1] 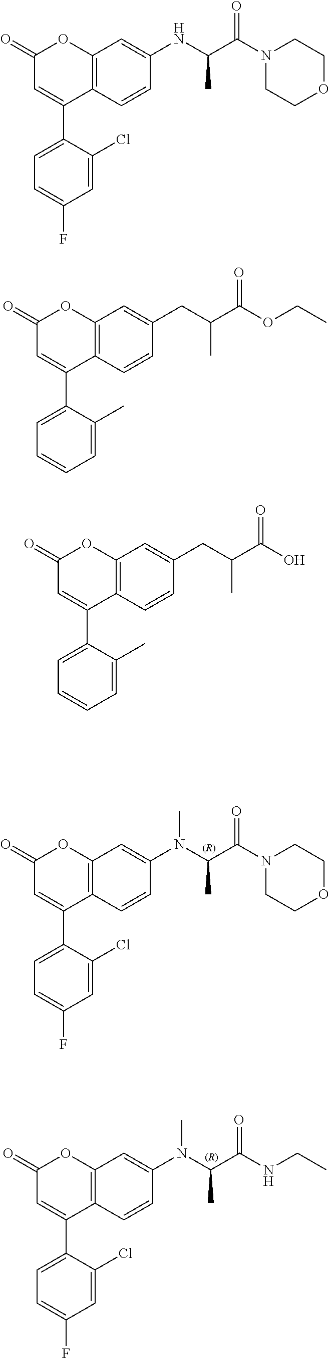 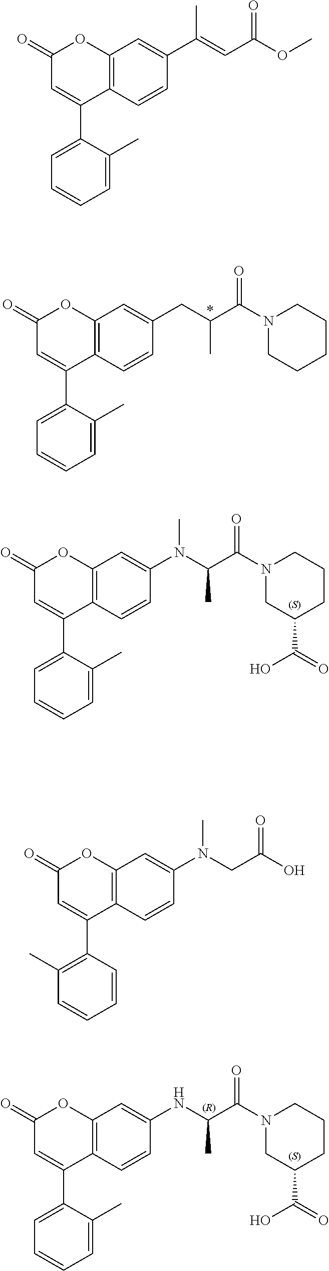 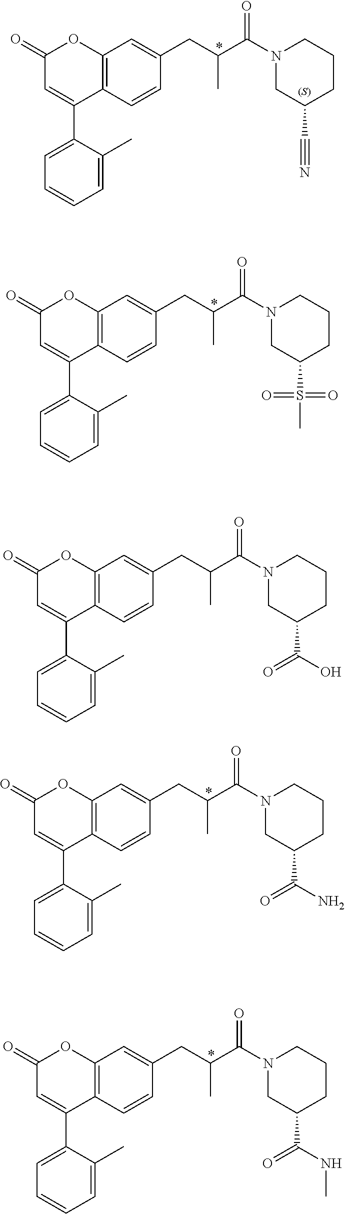 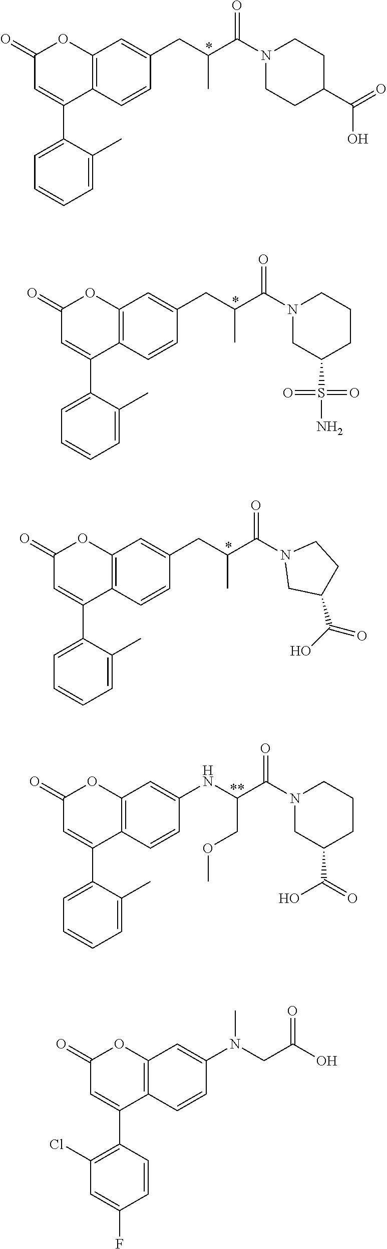 [SMILES: Cc1ccccc1-c1cc(=O)oc2cc(CC(C)C(=O)N3CCC(C(=O)O)CC3)ccc12 ; Cc1ccccc1-c1cc(=O)oc2cc(CC(C)C(=O)N3CCC[C@H](S(N)(=O)=O)C3)ccc12 ; Cc1ccccc1-c1cc(=O)oc2cc(CC(C)C(=O)N3CC[C@H](C(=O)O)C3)ccc12 ; COCC(Nc1ccc2c(-c3ccccc3C)cc(=O)oc2c1)C(=O)N1CCC[C@H](C(=O)O)C1 ; CN(CC(=O)O)c1ccc2c(-c3ccc(F)cc3Cl)cc(=O)oc2c1] 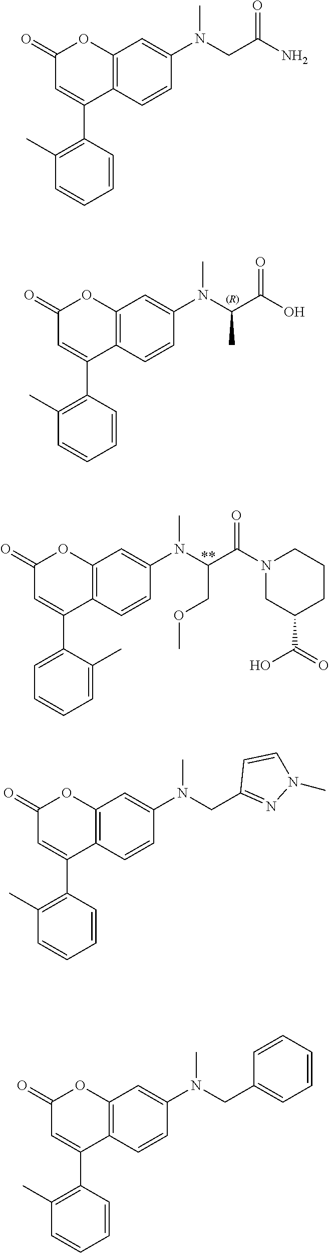 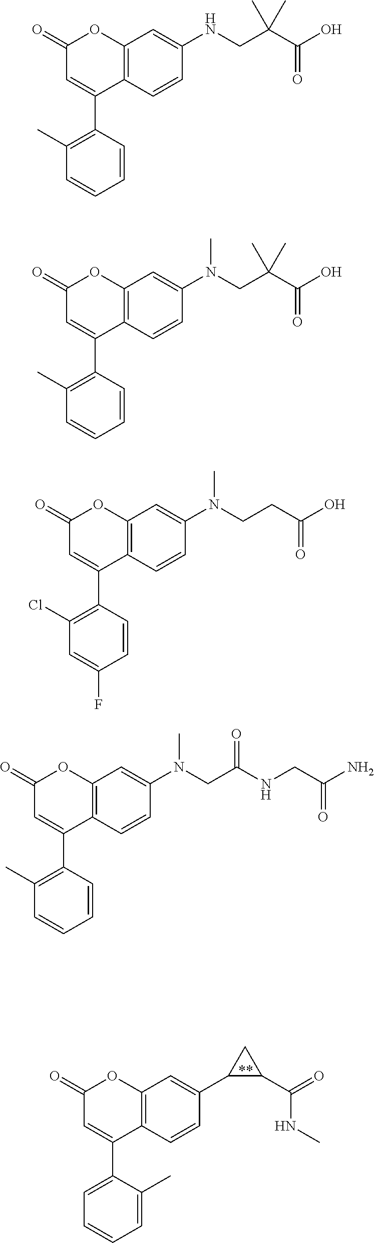 [SMILES: Cc1ccccc1-c1cc(=O)oc2cc(NCC(C)(C)C(=O)O)ccc12 ; Cc1ccccc1-c1cc(=O)oc2cc(N(C)CC(C)(C)C(=O)O)ccc12 ; CN(CCC(=O)O)c1ccc2c(-c3ccc(F)cc3Cl)cc(=O)oc2c1 ; Cc1ccccc1-c1cc(=O)oc2cc(N(C)CC(=O)NCC(N)=O)ccc12 ; CNC(=O)C1CC1c1ccc2c(-c3ccccc3C)cc(=O)oc2c1] 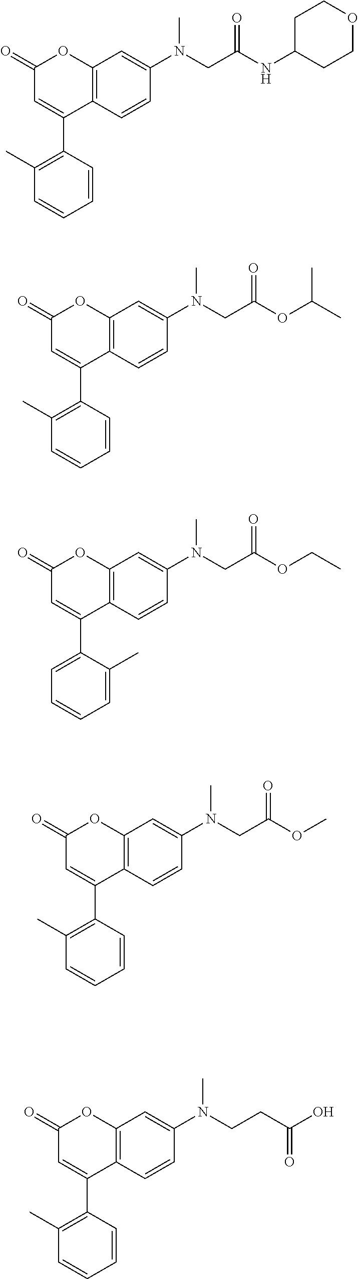 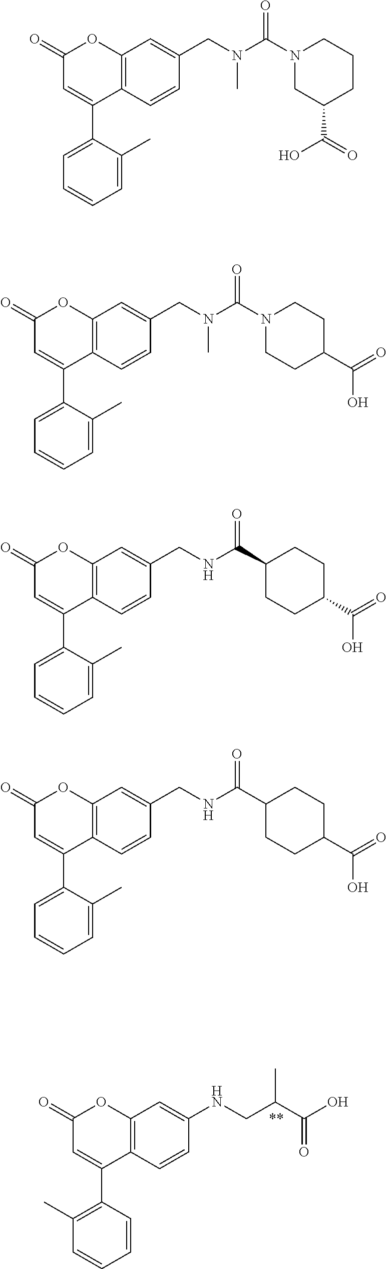 [SMILES: Cc1ccccc1-c1cc(=O)oc2cc(CN(C)C(=O)N3CCC[C@H](C(=O)O)C3)ccc12 ; Cc1ccccc1-c1cc(=O)oc2cc(CN(C)C(=O)N3CCC(C(=O)O)CC3)ccc12 ; Cc1ccccc1-c1cc(=O)oc2cc(CNC(=O)[C@H]3CC[C@H](C(=O)O)CC3)ccc12 ; Cc1ccccc1-c1cc(=O)oc2cc(CNC(=O)C3CCC(C(=O)O)CC3)ccc12 ; Cc1ccccc1-c1cc(=O)oc2cc(NCC(C)C(=O)O)ccc12] 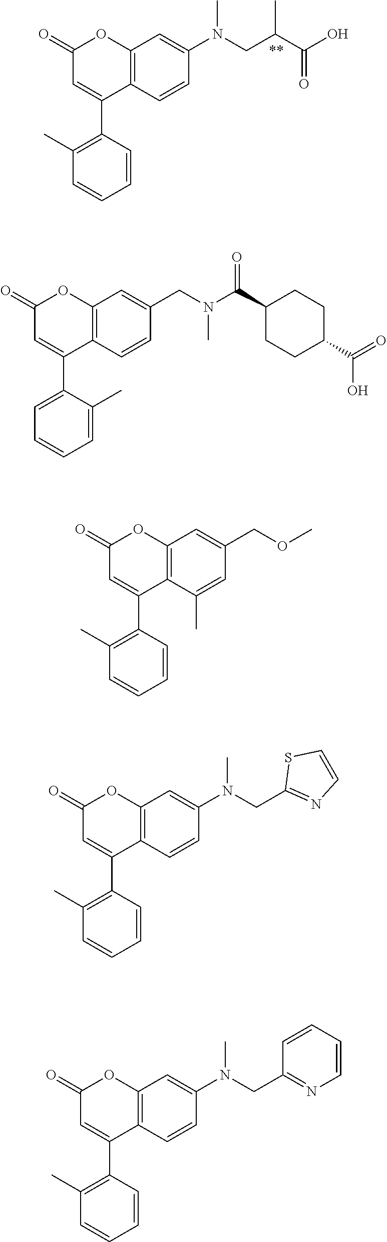 [SMILES: Cc1ccccc1-c1cc(=O)oc2cc(N(C)CC(C)C(=O)O)ccc12 ; Cc1ccccc1-c1cc(=O)oc2cc(CN(C)C(=O)[C@H]3CC[C@H](C(=O)O)CC3)ccc12 ; COCc1cc(C)c2c(-c3ccccc3C)cc(=O)oc2c1 ; Cc1ccccc1-c1cc(=O)oc2cc(N(C)Cc3nccs3)ccc12 ; Cc1ccccc1-c1cc(=O)oc2cc(N(C)Cc3ccccn3)ccc12] 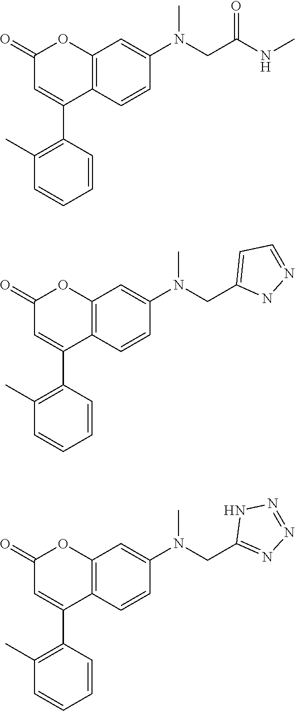 [SMILES: CNC(=O)CN(C)c1ccc2c(-c3ccccc3C)cc(=O)oc2c1 ; Cc1ccccc1-c1cc(=O)oc2cc(N(C)Cc3ccn[nH]3)ccc12 ; Cc1ccccc1-c1cc(=O)oc2cc(N(C)Cc3nnn[nH]3)ccc12] 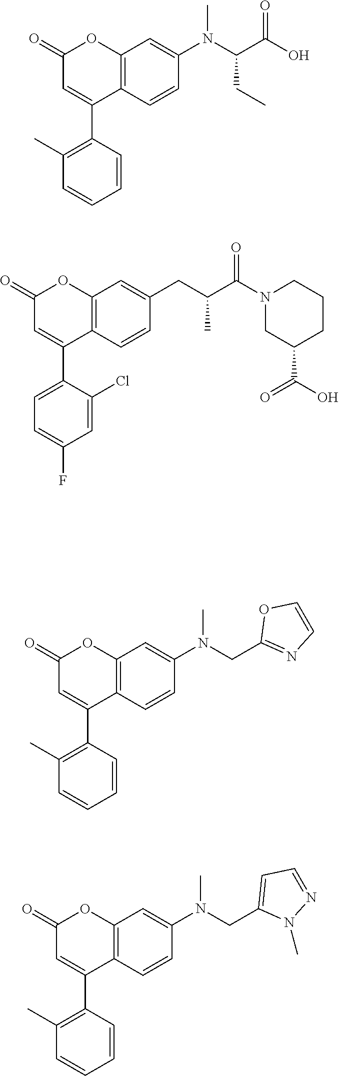 [SMILES: CC[C@@H](C(=O)O)N(C)c1ccc2c(-c3ccccc3C)cc(=O)oc2c1 ; C[C@H](Cc1ccc2c(-c3ccc(F)cc3Cl)cc(=O)oc2c1)C(=O)N1CCC[C@H](C(=O)O)C1 ; Cc1ccccc1-c1cc(=O)oc2cc(N(C)Cc3ncco3)ccc12 ; Cc1ccccc1-c1cc(=O)oc2cc(N(C)Cc3ccnn3C)ccc12] 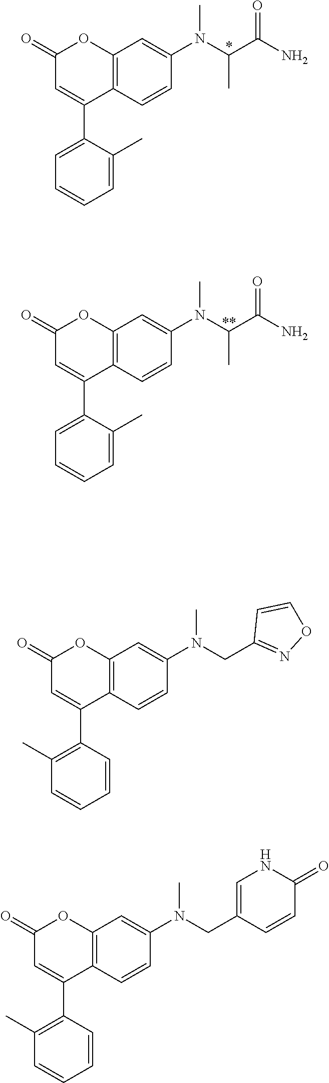 [SMILES: Cc1ccccc1-c1cc(=O)oc2cc(N(C)C(C)C(N)=O)ccc12 ; Cc1ccccc1-c1cc(=O)oc2cc(N(C)C(C)C(N)=O)ccc12 ; Cc1ccccc1-c1cc(=O)oc2cc(N(C)Cc3ccon3)ccc12 ; Cc1ccccc1-c1cc(=O)oc2cc(N(C)Cc3ccc(=O)[nH]c3)ccc12] 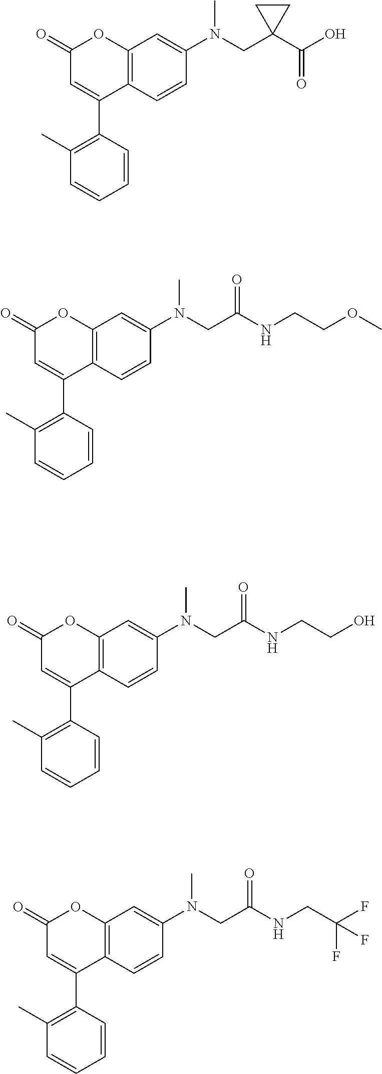 [SMILES: Cc1ccccc1-c1cc(=O)oc2cc(N(C)CC3(C(=O)O)CC3)ccc12 ; COCCNC(=O)CN(C)c1ccc2c(-c3ccccc3C)cc(=O)oc2c1 ; Cc1ccccc1-c1cc(=O)oc2cc(N(C)CC(=O)NCCO)ccc12 ; Cc1ccccc1-c1cc(=O)oc2cc(N(C)CC(=O)NCC(F)(F)F)ccc12] 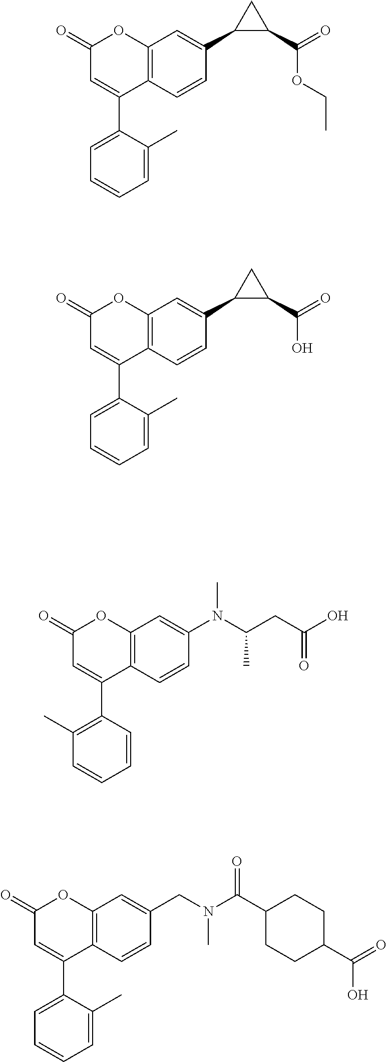 [SMILES: CCOC(=O)[C@@H]1C[C@@H]1c1ccc2c(-c3ccccc3C)cc(=O)oc2c1 ; Cc1ccccc1-c1cc(=O)oc2cc([C@H]3C[C@H]3C(=O)O)ccc12 ; Cc1ccccc1-c1cc(=O)oc2cc(N(C)[C@@H](C)CC(=O)O)ccc12 ; Cc1ccccc1-c1cc(=O)oc2cc(CN(C)C(=O)C3CCC(C(=O)O)CC3)ccc12] 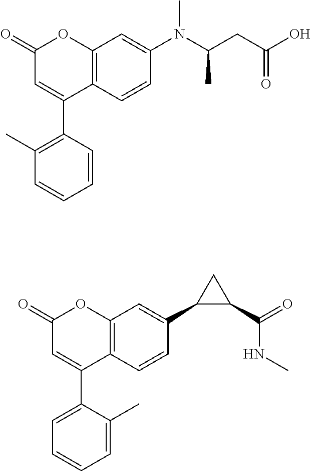 [SMILES: Cc1ccccc1-c1cc(=O)oc2cc(N(C)[C@H](C)CC(=O)O)ccc12 ; CNC(=O)[C@@H]1C[C@@H]1c1ccc2c(-c3ccccc3C)cc(=O)oc2c1]
or a pharmaceutically acceptable salt thereof.
